SEQUENCE LISTING (3 sequences)

SEQ ID NO: 1 - protein, 9 aa, synthetic construct
IPVAIKELR

SEQ ID NO: 2 - protein, 8 aa, synthetic construct
MQLMPFGS

SEQ ID NO: 3 - protein, 4 aa, synthetic construct
TDFG

(19) United States
(12) Patent Application Publication
(10) Pub. No.: US 2024/0409609 A1
(43) Pub. Date: Dec. 12, 2024

(54) REVERSIBLE LYSINE COVALENT MODIFIERS OF EGFR AND USES THEREOF

(71) Applicant: Terremoto Biosciences, Inc., San Francisco, CA (US)

(72) Inventors: Peter A. Thompson, Kirkland, WA (US); Kin S. Yang, San Mateo, CA (US); Solomon H. Reisberg, San Francisco, CA (US); Hang Chu, San Mateo, CA (US); Jordan D. Carelli, San Francisco, CA (US)

(21) Appl. No.: 18/640,370

(22) Filed: Apr. 19, 2024

Related U.S. Application Data
(63) Continuation of application No. PCT/US2022/078557, filed on Oct. 21, 2022.
(60) Provisional application No. 63/370,997, filed on Aug. 10, 2022.
(60) Provisional application No. 63/270,988, filed on Oct. 22, 2021.

(51) Int. Cl. C07K 14/71; C07D 401/12; C07D 401/14; C07D 403/04; C07F 9/6558; C07K 1/107

(52) U.S. Cl. CPC C07K 14/71; C07D 401/12; C07D 401/14; C07D 403/04; C07F 9/65583; C07K 1/1077

(57) ABSTRACT

EGFR is a frequently overexpressed protein in certain human cancers. Disclosed herein are compositions and methods for the modulation EGFR proteins, specifically EGFR mutants comprising single, double, or triple mutations.

### CROSS-REFERENCE TO RELATED APPLICATIONS

This application is a continuation application of International Patent Application No. PCT/US2022/078557, filed Aug. 21, 2022, which claims the benefit of U.S. Provisional Application No. 63/270,988 filed on Oct. 22, 2021, and U.S. Provisional Application No. 63/370,997 filed on Aug. 10, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 18, 2024, is named 61402-702_301_SeqList_ST26.xml and is 3,731 bytes in size.

### BACKGROUND OF THE INVENTION

Epidermal Growth Factor Receptor (EGFR) is a member of the type 1 tyrosine kinase family of growth factor receptors. These receptors play critical roles in cellular growth, differentiation, and survival. Activation of these receptors typically occurs via specific ligand binding, resulting in hetero- or homodimerization between receptor family members. The activation triggers a cascade of intracellular signaling pathways involved in both cellular proliferation and survival.

Overexpression of EGFR is present in at least 70% of human cancers (Seymour, L. K., Curr Drug Targets 2, 2001, pp. 117-133) such as, non-small cell lung cancer (NSCLC). EGFR-activating mutations, such as an exon 19 deletion (Dell9) and L858R substitution, have been reported in 10% to 50% of patients with NSCLC (Yu, H. A. et al. Clin Cancer Res 2013, 19, pp. 2240-2247). First and second-generation EGFR inhibitors were the standard-of-care for advanced EGFR-mutant NSCLC. However, patients invariably acquired resistance after treatment due to a single nucleotide substitution resulting in a T790M mutation. Currently, Osimertnib is a third generation EGFR inhibitor which inhibits the effects of EGFR-activating mutations with or without T790M. However, the C797S mutation is reportedly the most common mechanism underlying osimertinib resistance in patients (Ramalingam S. S. et al. J Clin Oncol 2018, 36, pp. 841-849). Thus, there is a need for a new generation of EGFR inhibitors capable of combating EGFR double and triple mutations present in cancers.

### SUMMARY OF THE INVENTION

In certain aspects, the present disclosure provides an EGFR protein covalently bound to a compound, wherein the compound is covalently bound to a lysine residue of the EGFR protein, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188. In some embodiments, the EGFR protein is an EGFR mutant with at least one mutation selected from Del19, T790M, C797S, and L858R. In some embodiments, wherein the EGFR mutant is selected from Del19/C797S, Del19/T790M, L858R/C797S, L858R/T790M, Del19/C797S/T790M, and L858R/C797S/T790M. In some embodiments, the EGFR mutant is selected from Del19/C797S, Del19/T790M, L858R/C797S, and L858R/T790M. In some embodiments, the EGFR mutant is selected from Dell9/C797S/T790M and L858R/C797S/T790M. In some embodiments, the lysine residue is K745. In some embodiments, the EGFR protein is in vivo. In some embodiments, the EGFR protein is an in vivo engineered EGFR protein, wherein the in vivo engineered EGFR protein is generated by contacting the EGFR protein in vivo with the compound by a nucleophilic addition reaction. In some embodiments, the in vivo engineered EGFR protein is a human in vivo engineered EGFR protein. In some embodiments, the covalent bond between the compound and the lysine residue is a reversible covalent bond. In some embodiments, the reversible covalent bond in the in vivo EGFR protein is an imine bond. In some embodiments, the imine bond is between K745 and an aldehyde functional group. In some embodiments, the aldehyde functional group is an aromatic aldehyde. In some embodiments, tyrosine kinase activity of EGFR is reduced by at least 10% when compared to native EGFR in contact with osimertinib or a salt thereof.

In another aspect, the present disclosure provides an in vivo engineered EGFR protein comprising a non-naturally occurring reversible covalent modification at K745, the reversible covalent modification is generated from an in vivo nucleophilic reaction between an exogenous aromatic aldehyde and K745, wherein the exogenous aromatic aldehyde undergoes a nucleophilic addition with the nitrogen atom on K745 and forming an imine bond between the exogenous aromatic aldehyde and K745. In some embodiments, the in vivo engineered EGFR protein is a human in vivo engineered EGFR protein. In some embodiments, tyrosine kinase activity of EGFR is reduced by at least 10% when compared to native EGFR in contact with osimertinib or a salt thereof. In some embodiments, the in vivo engineered EGFR protein comprises at least one mutation selected from Del19, T790M, C797S, and L858R.

In another aspect, the present disclosure provides a method comprising contacting a lysine residue in EGFR with a reversible covalent inhibitor, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188. In some embodiments, the EGFR protein is an EGFR mutant with at least one mutation selected from Del19, T790M, C797S, and L858R. In some embodiments, the EGFR mutant is selected from Del19/C797S, Del19/T790M, L858R/C797S, L858R/T790M, Dell9/C797S/T790M, and L858R/C797S/T790M. In some embodiments, the EGFR mutant is selected from Del19/C797S, Del19/T790M, L858R/C797S, and L858R/T790M. In some embodiments, the EGFR mutant is selected from Del19/C797S/T790M and L858R/C797S/T790M. In some embodiments, the lysine residue is K745.

In some aspects, the present disclosure provides a compound that covalently binds to a lysine residue in EGFR, wherein the compound or a salt has a reversible covalent interaction with the lysine residue in EGFR, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188. In some embodiments, the lysine residue is K745. In some aspects, the EGFR is an EGFR mutant. In some embodiments, the compound is selected from osimertinb, mobocertinib, CH7233163, almonertinib, and lazertinib, any one of which is modified with a reversible covalent electrophile. In some embodiments, the compound is modified to not be reactive with a cysteine residue of EGFR.

In certain aspects, the present disclosure provides a compound represented by the structure of Formula (I):

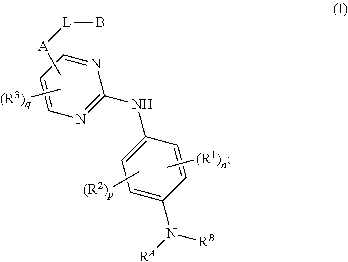

or a pharmaceutically acceptable salt thereof, wherein:
A is absent or selected from a monocyclic 5- to 6-membered heteroaryl, bicyclic heteroaryl, bicyclic aryl, and phenyl, any of which are optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN;
L is selected from: a bond, O, N(R¹⁵), S(R¹⁶); and C₁₋₆ alkylene optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
B is an amine-reactive lysine electrophile;
R¹ is an electrophile;
each R² is independently selected from: halogen, C₁₋₆ haloalkyl, C₁₋₆ hydroxyalkyl, C₁₋₆ alkoxy, C₁₋₆ haloalkoxy, —CN, —NO₂, and —NH₂;
each R³ is independently selected from:
   halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹², —S(O)₂N(R¹²)₂, —NO₂, and —CN; and
   C₁₋₆ alkyl, C₂₋₆ alkenyl, monocyclic 5- to 6-membered heteroaryl, bicyclic heteroaryl, bicyclic aryl, and phenyl, each of which is optionally substituted with one or more substituents independently selected from: halogen, —OR¹², —SR¹², —N(R¹²)₂, —C(O)R¹² —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹², —S(O)₂N(R¹²)₂, —NO₂, C₁₋₆ alkyl, C₁₋₆ haloalkyl, and —CN;
R^{A} and R^{B} are each independently selected from hydrogen; and C₁₋₆ alkyl optionally substituted with one or more substituents independently selected from: halogen, —OR¹³, —SR¹³ —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —NO₂, and —CN; or
R^{A} and R^{B} can come together to form a 4- to 10-membered heterocycle optionally substituted with one or more substituents independently selected from:
   halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
   C₁₋₆ alkyl optionally substituted with one or more substituents independently selected from: halogen, —OR¹⁴, —SR¹⁴, —N(R¹⁴)₂, —C(O)R¹⁴ —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
   C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴ C(O)OR¹⁴, —NO₂, and —CN;
R¹⁰, R¹¹, R¹², R¹³, R¹⁴, R¹⁵, and R¹⁶ are each independently selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy;
n is selected from 0 and 1;
p is selected from 0, 1, 2, and 3; and
q is selected from 0, 1, and 2.

In another aspect, the present disclosure provides a compound represented by the structure of Formula (IA):

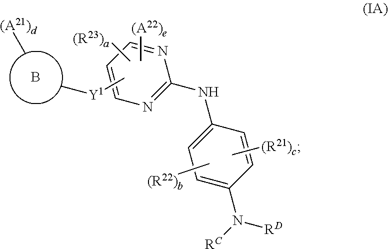

or a pharmaceutically acceptable salt thereof, wherein:
Y¹ is selected from a bond, —O—, —S—, and —N(R²⁴)—;
B is selected from a monocyclic 5- to 6-membered heteroaryl, bicyclic heteroaryl, bicyclic aryl, and phenyl, any of which is optionally substituted with one or more R²;
A²¹ is represented by -L²¹-C²¹;
A²² is represented by -L²²-C²²;
   L²¹ and L²² are each independently selected from a bond and -L^{A}-L^{B}-;
      L^{A} is selected from a bond, —O—, —S—, and —N(R²⁶)—; and
      L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹ —; N(R³¹)₂, —C(O)R³¹, —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), —S(O)R³¹, —S(O)₂R³¹, —S(O)₂N(R³¹)₂, ═O, ═S, ═NR³¹, —NO₂, and —CN;
   C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —S(O)R³², —S(O)₂R³², —S(O)₂N(R³²)₂, —NO₂, and —CN;
wherein at least one of C²¹, C²² and B is substituted by —C(O)H;
d is selected from 0 and 1;
e is selected from 0 and 1;
R²¹ is an electrophile;
R²² is independently selected at each occurrence from:
   halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, ═O, ═S, ═NR³⁴, —NO₂, and —CN;
R²³ is independently selected at each occurrence from:
   halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, ═O, ═S, ═NR³⁵, —NO₂, and —CN;
R²⁴ is selected from hydrogen, C₁₋₄ alkyl, and C₁₄ haloalkyl;
R²⁵ is independently selected at each occurrence from:
   halogen, —OR³⁶, —SR³⁶, —N(R³⁶)₂, —C(O)H, —C(O)R³⁷, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁶, —SR³⁶, —N(R³⁶)₂, —C(O)R³⁶, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, ═O, ═S, ═NR³⁶, —NO₂, and —CN;
R²⁶ is selected at each occurrence from hydrogen, C₁₋₄ alkyl, and C₁₄ haloalkyl;
a is selected from 0, 1, and 2.
b is selected from 0, 1, 2, 3, and 4;
c is selected from 0 and 1;
R^{C} and R^{D} are each independently selected from hydrogen and C₁₋₆ alkyl optionally substituted with one or more substituents independently selected from: halogen, —OR³⁸, —SR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), —S(O)R³⁸, —S(O)₂R³⁸, —S(O)₂N(R³⁸)₂, ═O, ═S, ═NR³⁸, —NO₂, and —CN; or
R^{C} and R^{D} come together to form a 4- to 10-membered heterocycle optionally substituted with one or more substituents independently selected from:
   halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
   C₁₋₆ alkyl optionally substituted with one or more substituents independently selected from: halogen, —OR³⁹, —SR³⁹, —N(R³⁹)₂, —C(O)R³⁹, —C(O)OR³⁹ —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹ —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy; and
   R³³ and R³⁷ are each independently selected at each occurrence from: C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In another aspect, the present disclosure provides a compound represented by the structure of Formula (VI):

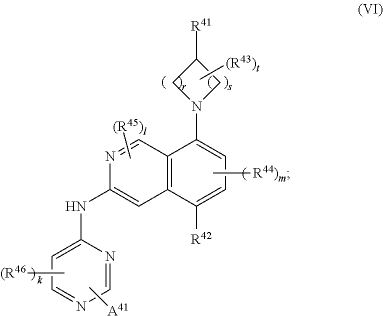

or a pharmaceutically acceptable salt thereof, wherein:
   A⁴¹ is represented by -L⁴¹-C⁴¹;
      L⁴¹ is selected from a bond and -L^{C}-L^{D}-;
         L^{C} is selected from absent, —O—, —S—, and —N(R⁴⁷)—; and
         L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹ —SR⁵¹ —N(R⁵¹)₂, —C(O)R⁵¹, —C(O)OR⁵¹, —OC(O)R⁵¹, —OC(O)N(R⁵¹)₂, —C(O)N(R⁵¹)₂, —N(R⁵¹)C(O)R⁵¹, —N(R⁵¹)C(O)OR⁵¹, —N(R⁵¹)C(O)N(R⁵¹)₂, —N(R⁵¹)S(O)₂(R⁵¹), —S(O)R⁵¹, —S(O)₂R⁵¹, —S(O)₂N(R⁵¹)₂, ═O, ═S, ═NR⁵¹, —NO₂, and —CN;
      C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —C(O)R⁵³, —C(O)OR⁵², —OC(O)R⁵², —OC(O)N(R⁵²)₂, —C(O)N(R⁵²)₂, —N(R⁵²)C(O)R¹², —N(R⁵²)C(O)OR¹², —N(R⁵²)C(O)N(R¹²)₂, —N(R⁵²)S(O)₂(R⁵²), —S(O)R¹², —S(O)₂R¹², —S(O)₂N(R¹²)₂, —NO₂, and —CN;
   R⁴¹ is selected from C₁₋₄ alkyl optionally substituted with one or more substituent independently selected at each occurrence from: halogen, —OR⁵⁴, —SR⁵⁴, —N(R¹⁴)₂, —C(O)R⁵⁴, —C(O)OR¹⁴, —OC(O)R¹⁴, —OC(O)N(R¹⁴)₂, —C(O)N(R⁵⁴)₂, —N(R⁵⁴)C(O)R⁵⁴, —N(R⁵⁴)C(O)OR⁵⁴, —N(R⁵⁴)C(O)N(R⁵⁴)₂, —N(R⁵⁴)S(O)₂(R⁵⁴), —S(O)R⁵⁴, —S(O)₂R⁵⁴, —S(O)₂N(R⁵⁴)₂, ═O, ═S, ═NR⁵⁴, —NO₂, and —CN;
   R⁴² is selected from C₁₋₆ alkyl optionally substituted with one or more substituent independently selected at each occurrence from: halogen, —OR⁵⁵, —SR⁵⁵ —N(R⁵⁵)₂, —C(O)R⁵⁵, —C(O)OR⁵⁵, —OC(O)R⁵⁵, —OC(O)N(R⁵⁵)₂, —C(O)N(R⁵⁵)₂, —N(R⁵⁵)C(O)R⁵⁵, —N(R⁵⁵)C(O)OR⁵⁵, —N(R⁵⁵)C(O)N(R⁵⁵)₂, —N(R⁵⁵)S(O)₂(R⁵⁵), —S(O)R⁵⁵, —S(O)₂R⁵⁵, —S(O)₂N(R⁵⁵)₂, ═O, ═S, ═NR⁵⁵, —NO₂, and —CN;
   R⁴³ is selected from halogen, C₁₋₆ alkyl, C₁₋₆ haloalkyl, —OR⁵⁶, —SR⁵⁶, —N(R⁵⁶)₂, —C(O)R⁵⁶, —C(O)OR⁵⁶, —OC(O)R⁵⁶, —OC(O)N(R⁵⁶)₂, —C(O)N(R⁵⁶)₂, —N(R⁵⁶)C(O)R⁵⁶, —N(R⁵⁶)C(O)OR⁵⁶, —N(R⁵⁶)C(O)N(R⁵⁶)₂, —N(R⁵⁶)S(O)₂(R⁵⁶), —S(O)R⁵⁶, —S(O)₂R⁵⁶, —S(O)₂N(R⁵⁶)₂, —NO₂, and —CN; or
   two R⁴³ attached to the same carbon atom together form ═O, ═S, and =NR⁵⁶;
   R⁴⁴, R⁴⁵, and R⁴⁶ are each independently selected at each occurrence from: halogen, C₁₋₆ alkyl, C₁₋₆ haloalkyl, —OR⁵⁷, —SR⁵⁷, —N(R⁵⁷)₂, —C(O)R⁵⁷, —C(O)OR⁵⁷, —OC(O)R⁵⁷, —OC(O)N(R⁵⁷)₂, —C(O)N(R⁵⁷)₂, —N(R⁵⁷)C(O)R⁵⁷, —N(R⁵⁷)C(O)OR⁵⁷, —N(R⁵⁷)C(O)N(R⁵⁷)₂, —N(R⁵⁷)S(O)₂(R⁵⁷), —S(O)R⁵⁷, —S(O)₂R⁵⁷, —S(O)₂N(R⁵⁷)₂, —NO₂, and —CN;
   R⁴⁷ is selected from hydrogen, C₁₋₆ alkyl, and C₁₋₆ haloalkyl;
   R⁵¹, R⁵², R⁵⁴, R⁵⁵, R⁵⁶, and R⁵⁷ are each independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy; and
   R⁵³ is selected at each occurrence from: C₁₋₆ alkyl, C₁₋₆ haloalkyl, C₁₋₆ hydroxyalkyl, C₁₋₆ alkoxy, and C₁₋₆ haloalkoxy;
   r and s are each independently selected from 1 and 2;
   t is selected from 0, 1, 2, 3, and 4; and
   k, l, and m are each independently selected from 0, 1, and 2.

In another aspect, the present disclosure provides a A compound represented by the structure of Formula (VIIA) or (VIIB):

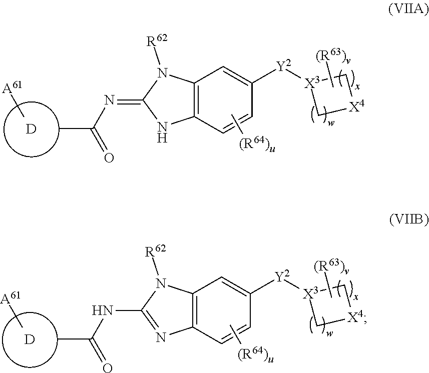

or a pharmaceutically acceptable salt thereof, wherein:
D is a phenyl or a 5- to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —OC(O)N(R⁷⁰)₂, —C(O)N(R⁷⁰)₂, —N(R⁷⁰)C(O)R⁷⁰, —N(R⁷⁰)C(O)OR⁷⁰, —N(R⁷⁰)C(O)N(R⁷⁰)₂, —N(R⁷⁰)S(O)₂(R⁷⁰), —S(O)R⁷⁰, —S(O)₂R⁷⁰, —S(O)₂N(R⁷⁰)₂, —NO₂, and —CN;
A⁶¹ is represented by *-L⁶¹-C⁶¹ wherein * represents the connection to D;
   L⁶¹ is selected from a bond and -L^{E}-L^{F}-;
      L^{E} is selected from absent, —O—, —S—, and —N(R⁶⁶)—; and
      L^{F} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), —S(O)R⁷¹, —S(O)₂R⁷¹, —S(O)₂N(R⁷¹)₂, ═O, ═S, ═NR⁷¹, —NO₂, and —CN;
   C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —S(O)R⁷², —S(O)₂R⁷², —S(O)₂N(R⁷²)₂, —NO₂, and —CN;
Y² is selected from a bond and C₁₋₄ alkylene optionally substituted with one or more substituents selected from halogen, —OH, —O—C₁₋₄ alkyl, ═O, and ═S;
X³ is CR⁶⁵ or N;
X⁴ is 0, C(R⁶⁵)(R⁶¹), or N(R⁶¹);
R⁶¹ is independently selected from:
   hydrogen;
   C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN; and
   5- to 6-membered heterocycle optionally substituted with one or more substituents selected from halogen, C₁₋₄ alkyl, C₁₄ haloalkyl, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN;
R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —OC(O)N(R⁷⁴)₂, —C(O)N(R⁷⁴)₂, —N(R⁷⁴)C(O)R⁷⁴, —N(R⁷⁴)C(O)OR⁷⁴, —N(R⁷⁴)C(O)N(R⁷⁴)₂, —N(R⁷⁴)S(O)₂(R⁷⁴), —S(O)R⁷⁴, —S(O)₂R⁷⁴, —S(O)₂N(R⁷⁴)₂, ═O, ═S, ═NR⁷⁴, —NO₂, and —CN;
R⁶³ and R⁶⁴ are each independently selected at each occurrence from halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, —N(R⁷⁵)S(O)₂(R⁷⁵), —S(O)R⁷⁵, —S(O)₂R⁷⁵, —S(O)₂N(R⁷⁵)₂, —NO₂, and —CN;
R⁶⁵ is selected from hydrogen, halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, —N(R⁷⁵)S(O)₂(R⁷⁵), —S(O)R⁷⁵, —S(O)₂R⁷⁵, —S(O)₂N(R⁷⁵)₂, —NO₂, and —CN;
R⁶⁶ is selected from hydrogen, C₁₋₆ alkyl, and C₁₋₆ haloalkyl;
R⁷⁰, R⁷¹, R⁷², R⁷³, R⁷⁴, and R⁷⁵ are each independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle;
u is selected from 0, 1, 2, and 3.
v is selected from 0, 1, 2, 3, and 4; and
w and x are each independently selected from 1 and 2.

In another aspect, the present disclosure provides a compound represented by the structure of Formula (VIIIA) or (VIIIB):

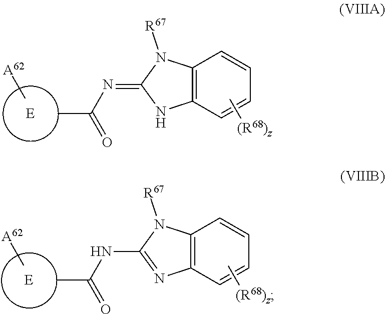

or a pharmaceutically acceptable salt thereof, wherein:
E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —OC(O)N(R⁷⁶)₂, —C(O)N(R⁷⁶)₂, —N(R⁷⁶)C(O)R⁷⁶, —N(R⁷⁶)C(O)OR⁷⁶, —N(R⁷⁶)C(O)N(R⁷⁶)₂, —N(R⁷⁶)S(O)₂(R⁷⁶), —S(O)R⁷⁶, —S(O)₂R⁷⁶, —S(O)₂N(R⁷⁶)₂, —NO₂, and —CN;
A⁶² is represented by *-L⁶²-C⁶², wherein * represents the connection to E;
   L⁶² is selected from a bond and -L^{G}-L^{H}-;
      L^{G} is selected from absent, —O—, —S—, and —N(R⁶⁹)—; and
      L^{H} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —OC(O)N(R⁷⁷)₂, —C(O)N(R⁷⁷)₂, —N(R⁷⁷)C(O)R⁷⁷, —N(R⁷⁷)C(O)OR⁷⁷, —N(R⁷⁷)C(O)N(R⁷⁷)₂, —N(R⁷⁷)S(O)₂(R⁷⁷), —S(O)R⁷⁷, —S(O)₂R⁷⁷, —S(O)₂N(R⁷⁷)₂, ═O, ═S, ═NR⁷⁷, —NO₂, and —CN;
   C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —OC(O)N(R⁷⁸)₂, —C(O)N(R⁷⁸)₂, —N(R⁷⁸)C(O)R⁷⁸, —N(R⁷⁸)C(O)OR⁷⁸, —N(R⁷⁸)C(O)N(R⁷⁸)₂, —N(R⁷⁸)S(O)₂(R⁷⁸), —S(O)R⁷⁸, —S(O)₂R⁷⁸, —S(O)₂N(R⁷⁸)₂, —NO₂, and —CN;
R⁶⁷ is independently selected from:
   C₁₋₆ alkyl, C₁₋₆ alkenyl, and C₁₋₆ alkynyl, each of which is optionally substituted with one or more substituents selected from:
      halogen, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN; and
      C₃₋₆ carbocycle, and 5- to 6-membered heterocycle, wherein the C₃₋₆ carbocycle and 5- to 6-membered heterocycle are each optionally substituted with one or more substituents selected from halogen, C₁₋₄ alkyl, C₁₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN;
   C₃₋₈ carbocycle and 3- to 8-membered heterocycle, each of which is optionally substituted with one or more substituents selected from halogen, C₁₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN;
R⁶⁸ are each independently selected at each occurrence from halogen, C₁₋₆ alkyl, C₁₋₆ haloalkyl, —OR⁸⁰, —SR⁸⁰, —N(R⁸⁰)₂, —C(O)R⁸⁰, —C(O)OR⁸⁰, —OC(O)R⁸⁰, —C(O)N(R⁸⁰)₂, —N(R⁸⁰)S(O)₂(R⁸⁰), —S(O)R⁸⁰, —S(O)₂R⁸⁰, —S(O)₂N(R⁸⁰)₂, —NO₂, and —CN;
R⁶⁹ is selected from hydrogen, C₁₋₆ alkyl, and C₁₋₆ haloalkyl;
R⁷⁹ is selected at each occurrence from:
   hydrogen;
   C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), —S(O)R⁸¹, —S(O)₂R⁸¹, —S(O)₂N(R⁸¹)₂, ═O, ═S, —NO₂, and —CN;
   C₃₋₆ carbocycle and 5- to 6-membered heterocycle, each of which is optionally substituted with one or more substituents selected from halogen, C₁₄ alkyl, C₁₋₄ haloalkyl, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), —S(O)R⁸¹, —S(O)₂R⁸¹, —S(O)₂N(R⁸¹)₂, ═O, ═S, —NO₂, and —CN;
R⁷⁶, R⁷⁷, R⁷⁸, R⁸⁰, and R⁸¹ are each independently selected at each occurrence from hydrogen, C₁₋₆ alkyl, and C₁₋₆ haloalkyl; and
z is selected from 0, 1, 2, and 3.

In some aspects, the present disclosure provides a pharmaceutical composition comprising a compound or salt of Formula (I), (IA), (II), (IIIA), (IIIB), (IVA), (IVB), (VA), (VB), (VI), (VIIA), (VIIB), (VIIIA), or (VIIIB) and at least one pharmaceutically acceptable excipient.

In some aspects, the present disclosure provides a method of inhibiting EGFR in a subject in need thereof, comprising administering to the subject a compound or salt of Formula (I), (IA), (II), (IIIA), (IIIB), (IVA), (IVB), (VA), (VB), (VI), (VIIA), (VIIB), (VIIIA), or (VIIIB) or a pharmaceutical composition thereof.

In some aspects, the present disclosure provides a method of treating cancer in a subject in need thereof, comprising administering to the subject a compound or salt of Formula (I), (IA), (II), (IIIA), (IIIB), (IVA), (IVB), (VA), (VB), (VI), (VIIA), (VIIB), (VIIIA), or (VIIIB) or a pharmaceutical composition thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates intact-protein mass spectra for the EGFR L858R/T790M/C797S triple-mutant protein incubated with Compound 1, demonstrating covalent complex formation.

FIG. 2 illustrates intact-protein mass spectra for the EGFR L858R/T790M/C797S triple-mutant protein incubated with Compound 2, demonstrating covalent complex formation.

FIG. 3A illustrates intact-protein mass spectra for the EGFR L858R/T790M/C797S triple-mutant protein incubated with Compound 3.

FIG. 3B illustrates intact-protein mass spectra for the EGFR L858R/T790M/C797S triple-mutant protein incubated with Compound 3 and a significant stoichiometric excess of a competitor molecule brigatinib, known to bind and inhibit EGFR.

FIG. 4A illustrates intact-protein mass spectra for the EGFR L858R/T790M/C797S triple-mutant protein incubated with Compound 4.

FIG. 4B illustrates intact-protein mass spectra for the EGFR L858R/T790M/C797S triple-mutant protein incubated with Compound 4 and a significant stoichiometric excess of the competitor molecule brigatinib.

FIG. 5A illustrates intact-protein mass spectra for the EGFR L858R/T790M/C797S triple-mutant protein incubated with Compound 5.

FIG. 5B illustrates intact-protein mass spectra for the EGFR L858R/T790M/C797S triple-mutant protein incubated with Compound 5 and a significant stoichiometric excess of the competitor molecule brigatinib.

FIG. 6A illustrates intact-protein mass spectra for the EGFR L858R/T790M/C797S triple-mutant protein incubated with Compound 6.

FIG. 6B illustrates intact-protein mass spectra for the EGFR L858R/T790M/C797S triple-mutant protein incubated with Compound 6 and a significant stoichiometric excess of the competitor molecule brigatinib.

FIG. 7A illustrates intact-protein mass spectra for the EGFR L858R/T790M/C797S triple-mutant protein incubated with Compound 7.

FIG. 7B illustrates intact-protein mass spectra for the EGFR L858R/T790M/C797S triple-mutant protein incubated with Compound 7 and a significant stoichiometric excess of the competitor molecule brigatinib.

FIG. 8A illustrates intact-protein mass spectra for the EGFR L858R/T790M/C797S triple-mutant protein incubated with Compound 8.

FIG. 8B illustrates intact-protein mass spectra for the EGFR L858R/T790M/C797S triple-mutant protein incubated with Compound 8 and a significant stoichiometric excess of the competitor molecule brigatinib.

FIG. 9A illustrates intact-protein mass spectra for the EGFR L858R/T790M/C797S triple-mutant protein incubated with reference compound brigatinib.

FIG. 9B illustrates intact-protein mass spectra for the EGFR L858R/T790M/C797S triple-mutant protein incubated with brigatinib and a significant stoichiometric excess of the competitor molecule brigatinib.

FIGS. 10A-10B provides intact mass spectra following incubation of Compound 3 with EGFR.

FIGS. 11A-11B provides intact mass spectra following incubation of Compound 3 with EGFR in the presence of a molar excess of competitor molecule BI-4020.

FIGS. 12A-12B provides intact mass spectra following incubation of Compound 2 with EGFR.

FIGS. 13A-13B provides intact mass spectra following incubation of Compound 2 with EGFR in the presence of a molar excess of competitor molecule BI-4020.

FIGS. 14A-14B provides intact mass spectra following incubation of Compound 1 with EGFR.

FIGS. 151-15B provides intact mass spectra following incubation of Compound 1 with EGFR in the presence of a molar excess of competitor molecule BI-4020.

FIGS. 16A-16B provides intact mass spectra following incubation of Compound 6 with EGFR.

FIGS. 17A-17B provides intact mass spectra following incubation of Compound 6 with EGFR in the presence of a molar excess of competitor molecule BI-4020.

FIGS. 18A-18B provides intact mass spectra following incubation of Compound 26 with EGFR.

FIGS. 19A-19B provides intact mass spectra following incubation of Compound 26 with EGFR in the presence of a molar excess of competitor molecule BI-4020.

FIGS. 20A-20B provides intact mass spectra following incubation of Compound 127 with EGFR.

FIGS. 21A-21B provides intact mass spectra following incubation of Compound 127 with EGFR in the presence of a molar excess of competitor molecule BI-4020.

FIGS. 22A-22B provides intact mass spectra following incubation of Compound 133 with EGFR.

FIGS. 23A-23B provides intact mass spectra following incubation of Compound 133 with EGFR in the presence of a molar excess of competitor molecule BI-4020.

FIGS. 24A-24B provides intact mass spectra following incubation of Compound 7 with EGFR.

FIGS. 25A-25B provides intact mass spectra following incubation of Compound 7 with EGFR in the presence of a molar excess of competitor molecule BI-4020.

FIGS. 26A-26B provides intact mass spectra following incubation of Compound 11 with EGFR.

FIGS. 27A-27B provides intact mass spectra following incubation of Compound 11 with EGFR in the presence of a molar excess of competitor molecule BI-4020.

FIGS. 28A-28B provides intact mass spectra following incubation of Compound 17 with EGFR.

FIGS. 29A-29B provides intact mass spectra following incubation of Compound 17 with EGFR in the presence of a molar excess of competitor molecule BI-4020.

FIGS. 30A-30B provides intact mass spectra following incubation of Compound 19 with EGFR.

FIGS. 31A-31B provides intact mass spectra following incubation of Compound 19 with EGFR in the presence of a molar excess of competitor molecule BI-4020.

FIGS. 32A-32B provides intact mass spectra following incubation of Compound 32 with EGFR.

FIGS. 33A-33B provides intact mass spectra following incubation of Compound 32 with EGFR in the presence of a molar excess of competitor molecule BI-4020.

FIGS. 34A-34B provides intact mass spectra following incubation of Compound 62 with EGFR.

FIGS. 35A-35B provides intact mass spectra following incubation of Compound 62 with EGFR in the presence of a molar excess of competitor molecule BI-4020.

FIGS. 36A-36B provides intact mass spectra following incubation of Compound 42 with EGFR.

FIGS. 37A-37B provides intact mass spectra following incubation of Compound 42 with EGFR in the presence of a molar excess of competitor molecule BI-4020.

FIGS. 38A-38B provides intact mass spectra following incubation of Compound 85 with EGFR.

FIGS. 39A-39B provides intact mass spectra following incubation of Compound 85 with EGFR in the presence of a molar excess of competitor molecule BI-4020.

FIGS. 40A-40B provides intact mass spectra following incubation of Compound 64 with EGFR.

FIGS. 41A-41B provides intact mass spectra following incubation of Compound 64 with EGFR in the presence of a molar excess of competitor molecule BI-4020.

FIGS. 42A-42B provides intact mass spectra following incubation of Compound 57 with EGFR.

FIGS. 43A-43B provides intact mass spectra following incubation of Compound 57 with EGFR in the presence of a molar excess of competitor molecule.

FIGS. 44A-44B provides intact mass spectra following incubation of Compound 32 with EGFR.

FIGS. 45A-45B provides intact mass spectra following incubation of Compound 32 with EGFR in the presence of a molar excess of competitor molecule BI-4020.

FIGS. 46A-46B provides intact mass spectra following incubation of Compound 41 with EGFR.

FIGS. 47A-47B provides intact mass spectra following incubation of Compound 41 with EGFR in the presence of a molar excess of competitor molecule BI-4020.

FIGS. 48A-48B provides intact mass spectra following incubation of Compound 83 with EGFR.

FIGS. 49A-49B provides intact mass spectra following incubation of Compound 83 with EGFR in the presence of a molar excess of competitor molecule BI-4020.

FIGS. 50A-50B provides intact mass spectra following incubation of Compound 79 with EGFR.

FIGS. 51A-51B provides intact mass spectra following incubation of Compound 79 with EGFR in the presence of a molar excess of competitor molecule BI-4020.

FIGS. 52A-52B provides intact mass spectra following incubation of Compound 44 with EGFR.

FIGS. 53A-53B provides intact mass spectra following incubation of Compound 44 with EGFR in the presence of a molar excess of competitor molecule BI-4020.

FIGS. 54A-54B provides intact mass spectra following incubation of Compound 87 with EGFR.

FIGS. 55A-55B provides intact mass spectra following incubation of Compound 87 with EGFR in the presence of a molar excess of competitor molecule BI-4020.

FIGS. 56A-56B provides intact mass spectra following incubation of Compound 52 with EGFR.

FIGS. 57A-57B provides intact mass spectra following incubation of Compound 52 with EGFR in the presence of a molar excess of competitor molecule BI-4020.

FIGS. 58A-58B provides intact mass spectra following incubation of Compound 84 with EGFR.

FIGS. 59A-59B provides intact mass spectra following incubation of Compound 84 with EGFR in the presence of a molar excess of competitor molecule BI-4020.

FIGS. 60A-60B provides intact mass spectra following incubation of Compound 126 with EGFR.

FIGS. 61A-61B provides intact mass spectra following incubation of Compound 126 with EGFR in the presence of a molar excess of competitor molecule BI-4020.

FIGS. 62A-62B provides intact mass spectra following incubation of Compound 51 with EGFR.

FIGS. 63A-63B provides intact mass spectra following incubation of Compound 51 with EGFR in the presence of a molar excess of competitor molecule BI-4020.

FIGS. 64A-64B provides intact mass spectra following incubation of Compound 125 with EGFR.

FIGS. 65A-65B provides intact mass spectra following incubation of Compound 125 with EGFR in the presence of a molar excess of competitor molecule BI-4020.

FIG. 66A provides a table of m/z values observed for different peptide fragments of EGFR comprising K745.

FIG. 66B demonstrates sodium borohydride reduction of the reversible covalent complex between EGFR and a compound of the present disclosure.

FIG. 66C depicts the nonapeptide sequence comprising K745 and labeled fragmentation patterns. Figure discloses “IPVAIKELR” as SEQ ID NO: 1.

FIG. 67A depicts a mass spectrum observed after the incubation of Compound 64 with EGFR, followed by sodium borohydride reduction, trypsin digest, and MS analysis.

FIG. 67B provides a table of m/z values observed.

FIG. 68A depicts a mass spectrum observed after the incubation of Compound 7 with EGFR, followed by sodium borohydride reduction, trypsin digest, and MS analysis.

FIG. 68B provides a table of m/z values observed.

FIG. 69A depicts a mass spectrum observed after the incubation of Compound 8 with EGFR, followed by sodium borohydride reduction, trypsin digest, and MS analysis.

FIG. 69B provides a table of m/z values observed.

FIG. 70A depicts a mass spectrum observed after the incubation of Compound 3 with EGFR, followed by sodium borohydride reduction, trypsin digest, and MS analysis.

FIG. 70B provides a table of m/z values observed

FIG. 71A depicts the electron density map of Compound 85 co-crystalized with EGFR protein.

FIG. 71B depicts the electron density map of Compound 64 co-crystalized with EGFR protein.

FIG. 72A is a close-up from the crystal structures depicted in FIG. 71A, and FIG. 72B provides a 2-D diagram detailing the interactions between Compound 85 and residues of the EGFR protein. Figure discloses SEQ ID NOS 2 and 3, respectively, in order of appearance.

FIG. 73A is a close-up from the crystal structures depicted in FIG. 71B, and FIG. 73B provides a 2-D diagram detailing the interactions between Compound 64 and residues of the EGFR protein. Figure discloses SEQ ID NO: 2.

FIG. 74 illustrates kinetic off-rate analysis using jump dilution for Compound 1 incubated with EGFR dell9/T790M/C797S triple mutant, EGFR L858R/T790M/C797S triple mutant, and EGFR wild-type proteins.

FIG. 75 illustrates kinetic off-rate analysis using jump dilution for Compound 3 incubated with EGFR dell9/T790M/C797S triple mutant, EGFR L858R/T790M/C797S triple mutant, and EGFR wild-type proteins.

FIG. 76 illustrates kinetic off-rate analysis using jump dilution for Compound 6 incubated with EGFR dell9/T790M/C797S triple mutant, EGFR L858R/T790M/C797S triple mutant, and EGFR wild-type proteins.

FIG. 77 illustrates kinetic off-rate analysis using jump dilution for Compound 7 incubated with EGFR dell9/T790M/C797S triple mutant, EGFR L858R/T790M/C797S triple mutant, and EGFR wild-type proteins.

FIG. 78 illustrates kinetic off-rate analysis using jump dilution for Compound 8 incubated with EGFR dell9/T790M/C797S triple mutant, EGFR L858R/T790M/C797S triple mutant, and EGFR wild-type proteins.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (1.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈alkyl). In other embodiments, an alkyl comprises two to five carbon atoms (i.e., C₂-C₅ alkyl). In other embodiments, an alkyl comprises three to five carbon atoms (i.e., C₃-C₈ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₅ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₅ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a divalent hydrocarbon chain linking the rest of the molecule to a radical group, consisting solely of carbon and hydrogen, containing no unsaturation, and preferably having from one to twelve carbon atoms, for example, methylene, (methyl)methylene, ethylene, propylene, butylene, and the like. The alkylene chain is attached to the rest of the molecule through a single bond and to the radical group through a single bond. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —Cₓ.y alkylene- refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C-y alkenylene- refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term -Cx-yalkynylene-refers to a substituted or unsubstituted alkynylene chain with from x to y carbons in the alkynylene chain. For example, —C₂₋₆ alkynylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 3- to 12-membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quatemized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like.

Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro, 2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (=N—H), oximo (=N—OH), hydrazino (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{o}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂(where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo(═N—OH), hydrazine(═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R—O—R^{o}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine(═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{o}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2); and wherein each R^{b} is independently selected from a direct bond or a straight or branched alkylene, alkenylene, or alkynylene chain, and each R^{o} is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein “therapeutic effect,” encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “contacting” may include allowing two species to react, interact, or physically touch, for example the two species may be a compound as described herein and an EGFR protein or mutant thereof as described herein.

The term “epidermal growth factor receptor,” “EGFR,” and “EGFR protein” are used interchangeably herein and refer to the native sequence of EGFR, including mutants and/or variants thereof.

The term electrophile as used herein refers to a chemical moiety that is capable of accepting an electron pair (e.g. Lewis acid, electron pair acceptor). For example, an electrophile as used herein is a chemical moiety that accepts a pair of electrons thereby forming a covalent bond.

The term “amine-reactive electrophile” as used herein refers to a functional group on an exogenous compound (e.g., a compound of the present disclosure) that specifically reacts with the amine of a lysine residue thereby forming a covalent bond (e.g., carbon nitrogen double bond or sulfur-nitrogen single bond) between the nitrogen atom of the lysine side chain and the compound of the present disclosure. Examples of amine-reactive electrophiles include aldehydes (e.g., salicyl aldehydes) and sulfonyl fluorides (e.g., aromatic sulfonyl fluorides). Amine-reactive electrophiles can form covalent bonds with a lysine residue that is a stable covalent bond or a reversible covalent bond. In some embodiments, the amine-reactive electrophile forms a stable covalent bond between the nitrogen atom of the lysine side chain and a compound of the present disclosure. Such stable covalent bonds include bond that do not readily cleave under physiological conditions. In some embodiments, the amine-reactive electrophile forms a reversible covalent bond between the nitrogen atom of the lysine side chain and a compound of the present disclosure.

The term “reversible covalent bond” as used herein refers to a labile bond between the amine of a lysine residue and a compound as disclosed herein (e.g. between an electron deficient functional group and the amine of the lysine reside). As used herein a reversible covalent inhibitor or reversible covalent modifier, refer to classes of compounds that comprise a reversible covalent bond. The reversible covalent bond may be a bond as described herein (e.g., carbon nitrogen double bond). Bandyopadhyay, A. et al. Curr Opin Chem Biol. October 2016, (34) pp. 110-116; Serafimova, I. M. et al. Nat Chem Biol. May 2012, 8(5), pp. 471-476; and Bradshaw M. J. et al. Nat Chem Biol. Jul. 2015, 11(7): 525-531, describe reversible covalent modifiers or reversible covalent chemical kinetics, the entire contents of each of which are incorporated herein by reference.

### EGFR Compositions and Uses Thereof

In some aspects, the present disclosure provides an EGFR protein bound to a compound. In some embodiments, an EGFR protein is covalently bound to a compound. In some embodiments, an EGFR protein is covalently bound to a compound, wherein the compound is covalently bound to a lysine residue of the EGFR protein. In some embodiments, an EGFR protein is covalently bound to a compound, wherein the compound is covalently bound to a lysine residue of the EGFR protein selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188. In some embodiments, the lysine residue of the EGFR protein is K745.

In some embodiments, the EGFR protein is an EGFR mutant comprising a mutation selected from a single mutation, a double mutation, or a triple mutation. In some embodiments, the EGFR mutant comprises a single mutation. In some embodiments, the EGFR mutant comprises a double mutation. In some embodiments, the EGFR mutant comprises a triple mutation. In some embodiments, the EGFR mutant comprises a single mutation selected from Del19, T790M, C797S, and L858R. In some embodiments, the EGFR mutant comprises a double mutation selected from Del19/C797S, Del19/T790M, L858R/C797S, and L858R/T790M. In some embodiments, the EGFR mutant comprises a triple mutation selected from Dell9/C797S/T790M and L858R/C797S/T790M.

In some embodiments, the EGFR protein is an EGFR mutant with at least one mutation selected from Del19, T790M, C797S, and L858R. In some embodiments, the at least one mutation in the EGFR mutant is Del 19. In some embodiments, the at least one mutation in the EGFR mutant is T790M. In some embodiments, the at least one mutation in the EGFR mutant is C797S. In some embodiments, the at least one mutation in the EGFR mutant is L858R.

In some embodiments, the EGFR mutant comprises a mutation selected from Del19/C797S, Del19/T790M, L858R/C797S, L858R/T790M, Dell9/C797S/T790M, and L858R/C797S/T790M.

In some embodiments, the EGFR mutant is selected from Del19/C797S, Del19/T790M, L858R/C797S, and L858R/T790M. In some embodiments, the EGFR mutant is Del19/C797S. In some embodiments, the EGFR mutant is Del19/T790M. In some embodiments, the EGFR mutant is L858R/C797S. In some embodiments, the EGFR mutant is L858R/T790M. In some embodiments, the EGFR mutant is selected from Del19/C797S/T790M and L858R/C797S/T790M. In some embodiments, the EGFR mutant is Dell9/C797S/T790M. In some embodiments, the EGFR mutant is L858R/C797S/T790M.

In some embodiments, the EGFR protein is in vivo. In some embodiments, the EGFR protein is an in vivo engineered EGFR protein, wherein the in vivo engineered EGFR protein is generated by contacting the EGFR protein in vivo with the compound by a nucleophilic addition reaction. In some embodiments, the nucleophilic addition reaction produces an imine bond (e.g., a carbon-nitrogen double bond). In some embodiments, the nucleophilic addition reaction produces a sulfonamide (e.g., a sulfonyl group connected to an amine group, —S(O)₂—NH₂ as a non-limiting example). In some embodiments, the in vivo engineered EGFR protein is generated by contacting the EGFR protein with a compound containing a sulfonylfluoride functional group or aldehyde functional group. In some embodiments, the in vivo engineered EGFR protein is generated by contacting the EGFR protein with a compound containing a —S(O)₂F functional group or a —C(O)H functional group. In some embodiments, the in vivo engineered EGFR protein is a human in vivo engineered EGFR protein. In some embodiments, the covalent bond between the compound and the lysine residue is a reversible covalent bond. In some embodiments, the reversible covalent bond in the in vivo EGFR protein is an imine bond. In some embodiments, the imine bond is between K745 and an aldehyde functional group. In some embodiments, the aldehyde functional group is an aromatic aldehyde. In some embodiments, the covalent bond between the compound and the lysine residue is not a reversible covalent bond. In some embodiments, the covalent bond in the in vivo EGFR protein is sulfur-nitrogen bond. In some embodiments, the sulfur-nitrogen bond is between K745 and a sulfonyl functional group. In some embodiments, the sulfonyl functional group is an aromatic sulfonyl fluoride.

In some embodiments, tyrosine kinase activity of EGFR is reduced by at least 10% when compared to native EGFR in contact with osimertinib or a salt thereof. In some embodiments, tyrosine kinase activity of EGFR is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% when compared to native EGFR in contact with osimertinib or a salt thereof. In some embodiments, tyrosine kinase activity of EGFR is reduced by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% when compared to native EGFR in contact with osimertinib or a salt thereof. In some embodiments, tyrosine kinase activity of EGFR is reduced by 10%, 15%, 20%, 25%, 30%, 35%, 40%, 45%, 50%, 55%, 60%, 65%, 70%, 75%, 80%, 85%, 90%, or 95% when compared to native EGFR in contact with osimertinib or a salt thereof.

In some aspects, the present disclosure provides an in vivo engineered EGFR protein. In some embodiments, the in vivo engineered EGFR protein comprises a non-naturally occurring covalent modification at K745, the reversible covalent modification is generated from an in vivo nucleophilic reaction between an exogenous amine-reactive electrophile and K745, wherein the exogenous amine-reactive electrophile undergoes a nucleophilic addition with the nitrogen atom on K745 and forming a covalent bond between the amine-reactive electrophile and K745.

In some embodiments, the in vivo engineered EGFR protein comprises a non-naturally occurring reversible covalent modification at K745, the reversible covalent modification is generated from an in vivo nucleophilic reaction between an exogenous aromatic aldehyde and K745, wherein the exogenous aromatic aldehyde undergoes a nucleophilic addition with the nitrogen atom on K745 and forming an imine bond between the exogenous aromatic aldehyde and K745. In some embodiments, the in vivo engineered EGFR protein is a human in vivo engineered EGFR protein. In some embodiments, tyrosine kinase activity of EGFR is reduced by at least 10% when compared to native EGFR in contact with osimertinib or a salt thereof. In some embodiments, tyrosine kinase activity of EGFR is reduced by an amount as disclosed herein, when compared to native EGFR in contact with osimertinib or a salt thereof. In some embodiments, the in vivo engineered EGFR protein comprises at least one mutation selected from Del19, T790M, C797S, and L858R.

In some aspects, the present disclosure provides an in vivo engineered EGFR protein comprising a nitrogen-sulfur bond. In some embodiments, the in vivo engineered EGFR protein comprises a non-naturally occurring covalent modification at K745, the covalent modification is generated from an in vivo nucleophilic reaction between an exogenous aromatic sulfonyl fluoride and K745, wherein the exogenous aromatic aldehyde undergoes a nucleophilic addition with the nitrogen atom on K745 and forming a sulfur-nitrogen bond between the exogenous aromatic sulfonyl fluoride and K745. In some embodiments, the in vivo engineered EGFR protein is a human in vivo engineered EGFR protein. In some embodiments, tyrosine kinase activity of EGFR is reduced by at least 10% when compared to native EGFR in contact with osimertinib or a salt thereof. In some embodiments, tyrosine kinase activity of EGFR is reduced by an amount as disclosed herein, when compared to native EGFR in contact with osimertinib or a salt thereof. In some embodiments, the in vivo engineered EGFR protein comprises at least one mutation selected from Del19, T790M, C797S, and L858R.

In some aspects the present disclosure provides, a method comprising contacting a lysine residue in EGFR with an amine-reactive covalent inhibitor, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188.

In some aspects the present disclosure provides, a method comprising contacting a lysine residue in EGFR with a reversible covalent inhibitor, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188. In some embodiments, the EGFR protein is an EGFR mutant comprising a mutation selected from a single mutation, a double mutation, or a triple mutation. In some embodiments, the EGFR mutant comprises a single mutation. In some embodiments, the EGFR mutant comprises a double mutation. In some embodiments, the EGFR mutant comprises a triple mutation. In some embodiments, the EGFR mutant comprises a single mutation selected from Dell9, T790M, C797S, and L858R. In some embodiments, the EGFR mutant comprises a double mutation selected from Del19/C797S, Del19/T790M, L858R/C797S, and L858R/T790M. In some embodiments, the EGFR mutant comprises a triple mutation selected from Dell9/C797S/T790M and L858R/C797S/T790M. In some embodiments, the lysine residue is selected from K745.

In some embodiments, the EGFR protein is an EGFR mutant with at least one mutation selected from Del19, T790M, C797S, and L858R. In some embodiments, the at least one mutation in the EGFR mutant is Del 19. In some embodiments, the at least one mutation in the EGFR mutant is T790M. In some embodiments, the at least one mutation in the EGFR mutant is C797S. In some embodiments, the at least one mutation in the EGFR mutant is L858R.

In some embodiments, the EGFR mutant comprises a mutation selected from Del19/C797S, Del19/T790M, L858R/C797S, L858R/T790M, Dell9/C797S/T790M, and L858R/C797S/T790M.

In some embodiments, the EGFR mutant is selected from Del19/C797S, Del19/T790M, L858R/C797S, and L858R/T790M. In some embodiments, the EGFR mutant is Del19/C797S. In some embodiments, the EGFR mutant is Del19/T790M. In some embodiments, the EGFR mutant is L858R/C797S. In some embodiments, the EGFR mutant is L858R/T790M. In some embodiments, the EGFR mutant is selected from Del19/C797S/T790M and L858R/C797S/T790M. In some embodiments, the EGFR mutant is Dell9/C797S/T790M. In some embodiments, the EGFR mutant is L858R/C797S/T790M.

In some aspects, the present disclosure provides a compound that covalently binds to a lysine residue in EGFR, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188. In some embodiments, the lysine residue is K745. In some embodiments, the compound or a salt thereof has a reversible covalent interaction with the lysine residue in EGFR. In some embodiments, the EGFR protein is an EGFR mutant. In some embodiments, the EGFR mutant at least one mutation selected from Del19, T790M, C797S, and L858R. In some embodiments, the compound is modified with a reversible covalent electrophile. In some embodiments, the compound is selected from osimertinb, mobocertinib, CH7233163, almonertinib, and lazertinib, any one of which is modified with a reversible covalent electrophile. In some embodiments, the compound is modified to not be reactive with a cysteine residue of EGFR. In some embodiments, osimertinb, mobocertinib, CH7233163, almonertinib, and lazertinib are modified to not be reactive with a cysteine residue of EGFR.

### Compounds

In some aspects, the present disclosure provides a compound represented by the structure of Formula (I):

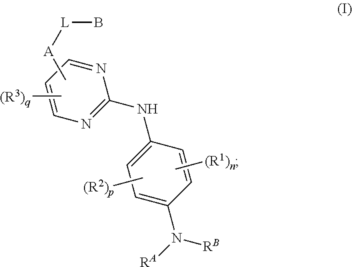

or a pharmaceutically acceptable salt thereof, wherein:
A is absent or -A¹-A²-;
   A¹ is absent or selected from —O—, —S—, and —N(R⁴)—;
   A² selected from a monocyclic 5- to 6-membered heteroarylene, bicyclic heteroarylene, bicyclic arylene, and phenylene, any of which are optionally substituted with one or more substituents selected from: halogen, —OR¹⁰, —N(R¹⁰)₂, —CN, —NO₂, and C₁₋₆ alkyl optionally substituted with one or more substituents independently selected from: halogen, —OR¹⁰, —N(R¹⁰)₂, —NO₂, and —CN;
L is absent or -L¹-L²-;
   L¹ is absent or selected from —O—, —N(R⁵)—, and —S—;
   L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
B is an amine-reactive lysine electrophile;
R¹ is an electrophile;
each R² is independently selected from: halogen, C₁₋₆ haloalkyl, C₁₋₆ hydroxyalkyl, C₁₋₆ alkoxy, C₁₋₆ haloalkoxy, —OR¹², —SR¹², —N(R¹²)₂, —C(O)OR¹², —OC(O)R¹², —C(O)N(R¹²)₂, —N(R¹²)C(O)R¹², —CN, and —NO₂;
each R³ is independently selected from:
   halogen, —OR¹³, —SR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —NO₂, and —CN; and
   C₁₋₆ alkyl, C₂₋₆ alkenyl, monocyclic 5- to 6-membered heteroaryl, bicyclic heteroaryl, bicyclic aryl, and phenyl, each of which is optionally substituted with one or more substituents independently selected from: halogen, —OR¹³, —SR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —P(O)(R¹³)₂, —NO₂, C₁₋₆ alkyl, C₁₋₆ haloalkyl, and —CN;
R⁴ and R⁵ are each independently selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy;
R^{A} and R^{B} are each independently selected from hydrogen; and C₁₋₆ alkyl optionally substituted with one or more substituents independently selected from: halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; or
R^{A} and R^{B} come together to form a 4- to 10-membered heterocycle optionally substituted with one or more substituents independently selected from:
   halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
   C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
   C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN;
R¹⁰, R¹¹, R¹², R¹³, and R¹⁴ are each independently selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy;
n is selected from 0 and 1;
p is selected from 0, 1, 2, and 3; and
q is selected from 0, 1, and 2.

In some embodiments, the present disclosure provides a compound represented by the structure of Formula (I):

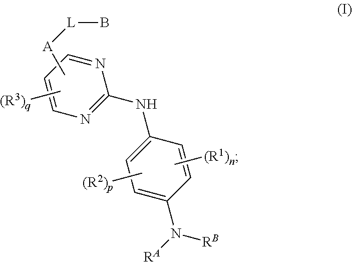

or a pharmaceutically acceptable salt thereof, wherein:
A is absent or -A¹-A²-;
   A¹ is absent or selected from —O— and —N(R⁴)—;
   A² selected from bicyclic heteroarylene and phenylene, any of which are optionally substituted with one or more substituents selected from: halogen, —OR¹⁰, —N(R¹⁰)₂, —CN, —NO₂, and C₁₋₆ alkyl optionally substituted with one or more substituents independently selected from: halogen, —OR¹⁰, —N(R¹⁰)₂, —NO₂, and —CN;
L is absent or -L¹-L²-;
   L¹ is absent;
   L² is absent or selected from C₁₋₆ alkylene and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
B is an amine-reactive lysine electrophile;
R¹ is an electrophile;
each R² is independently selected from: C₁₋₆ alkoxy and —N(R¹²)C(O)R¹²;
each R³ is independently selected from:
   halogen, —OR¹³, —N(R¹³)₂, and —N(R¹³)C(O)R¹³; and
   bicyclic heteroaryl and phenyl, each of which is optionally substituted with one or more substituents independently selected from: halogen, —OR¹³, —SR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —P(O)(R¹³)₂, —NO₂, C₁₋₆ alkyl, C₁₋₆ haloalkyl, and —CN;
R⁴ and R⁵ are each independently selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy;
R^{A} and R^{B} are each independently C₁₋₆ alkyl optionally substituted with one or more substituents independently selected from: halogen, —OR¹⁴, —SR¹⁴, —N(R¹⁴)₂, —C(O)R¹⁴ —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; or
R^{A} and R^{B} come together to form a 4- to 10-membered heterocycle optionally substituted with one or more substituents independently selected from:
   halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
   C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
   C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN;
R¹⁰, R¹¹, R¹², R¹³, and R¹⁴ are each independently selected at each occurrence from hydrogen, C₁₋₆ alkyl, and C₃₋₆ carbocycle, wherein the C₃₋₆ carbocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy;
n is 0;
p is selected from 1 and 2; and
q is selected from 0 and 1.

In some embodiments, the present disclosure provides a compound represented by the structure of Formula (I):

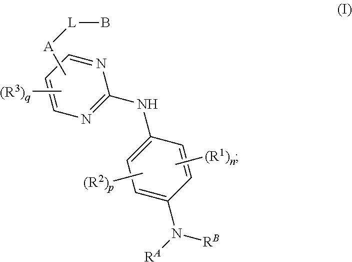

or a pharmaceutically acceptable salt thereof, wherein:
A is absent or -A¹-A²-;
   A¹ is absent or selected from —O— and —N(R⁴)—;
   A² selected from bicyclic heteroarylene and phenylene;
L is absent or -L¹-L²-;
   L¹ is absent;
   L² is absent or selected from C₁₋₆ alkylene and C₂₋₆ alkynylene;
B is an amine-reactive lysine electrophile;
R¹ is an electrophile;
each R² is independently selected from: C₁₋₆ alkoxy and —N(R¹²)C(O)R¹²;
each R³ is independently selected from:
   halogen, —OR¹³, —N(R¹³)₂, and —N(R¹³)C(O)R¹³; and
   bicyclic heteroaryl and phenyl, each of which is optionally substituted with one or more substituents independently selected from: —P(O)(R¹³)₂ and C₁₋₆ alkyl;
R⁴ and R⁵ are each independently selected from hydrogen and C₁₋₆ alkyl;
R^{A} and R^{B} are each independently C₁₋₆ alkyl optionally substituted with one or more substituents independently selected from: —N(R¹⁴)₂; or
R^{A} and R^{B} come together to form a 4- to 10-membered heterocycle optionally substituted with one or more substituents independently selected from: 3- to 10-membered heterocycle optionally substituted with one or more substituents independently selected from: C₁₋₆ alkyl;
R¹⁰, R¹¹, R¹², R¹³, and R¹⁴ are each independently selected at each occurrence from hydrogen,
C₁₋₆ alkyl, and C₃₋₆ carbocycle;
n is 0;
p is selected from 1 and 2; and
q is selected from 0 and 1.

In some embodiments, the present disclosure provides a compound represented by the structure of Formula (I):

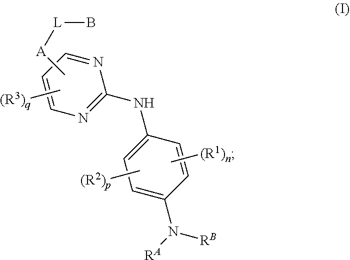

or a pharmaceutically acceptable salt thereof, wherein:
A is -A¹-A²-;
   A¹ is absent;
   A² selected from bicyclic heteroarylene optionally substituted with one or more substituents selected from: halogen, —OR¹⁰, —N(R¹⁰)₂, —CN, —NO₂, and C₁₋₆ alkyl optionally substituted with one or more substituents independently selected from: halogen, —OR¹⁰, —N(R¹⁰)₂, —NO₂, and —CN;
L is -L¹-L²-;
   L¹ is absent;
   L² is absent or C₁₋₆ alkylene optionally substituted with one or more substituents independently selected from: halogen, —OR¹¹, —SR¹¹, —N(R¹¹)₂, —C(O)R¹¹ —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
B is an amine-reactive lysine electrophile;
R¹ is an electrophile;
each R² is independently selected from: —N(R¹²)C(O)R¹²;
each R³ is independently selected from:
   halogen and —N(R¹³)C(O)R¹³; and
   bicyclic heteroaryl optionally substituted with one or more substituents independently selected from: halogen, —OR¹³, —SR¹³, —N(R¹³)₂, —C(O)R¹³, —C(O)OR¹³ —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —NO₂, C₁₋₆ alkyl, C₁₋₆ haloalkyl, and —CN;
R⁴ and R⁵ are each independently selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy;
R^{A} and R^{B} are each independently C₁₋₆ alkyl optionally substituted with one or more substituents independently selected from: halogen, —OR¹⁴, —SR¹⁴, —N(R¹⁴)₂, —C(O)R¹⁴ —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴ —S(O)₂N(R¹⁴)₂, —NO₂, and —CN;
R¹⁰, R¹¹, R¹², R¹³, and R¹⁴ are each independently selected at each occurrence from hydrogen and C₁₋₆ alkyl;
n is 0;
p is selected from 1 and 2; and
q is selected from 0 and 1.

In some embodiments, the present disclosure provides a compound represented by the structure of Formula (I):

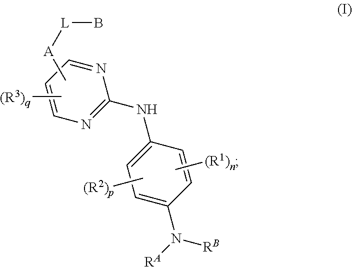

or a pharmaceutically acceptable salt thereof, wherein:
A is -A¹-A²-;
   A¹ is absent;
   A² is bicyclic heteroarylene;
L is -L¹-L²-;
   L¹ is absent;
   L² is absent or C₁₋₆ alkylene;
B is an amine-reactive lysine electrophile;
R¹ is an electrophile;
each R² is independently selected from: —N(R¹²)C(O)R¹²;
each R³ is independently selected from:
   halogen and —N(R¹³)C(O)R¹³; and
   bicyclic heteroaryl optionally substituted with one or more substituents independently selected from: C₁₋₆ alkyl;
R⁴ and R⁵ are each independently selected from hydrogen and C₁₋₆ alkyl;
R^{A} and R^{B} are each independently C₁₋₆ alkyl optionally substituted with one or more substituents independently selected from: —N(R¹⁴)₂; or
R¹⁰, R¹¹, R¹², R¹³, and R¹⁴ are each independently selected at each occurrence from hydrogen and C₁₋₆ alkyl;
n is 0;
p is selected from 1 and 2; and
q is selected from 0 and 1.

In some embodiments, the present disclosure provides a compound represented by the structure of Formula (I):

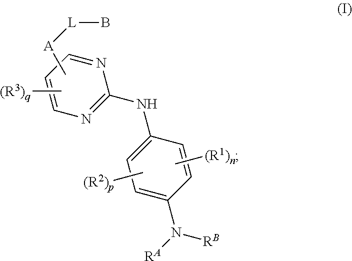

or a pharmaceutically acceptable salt thereof, wherein:
A is absent or -A¹-A²-;
   A¹ is selected from —O— and —N(R⁴)—;
   A² is selected from phenylene optionally substituted with one or more substituents selected from: halogen, —OR¹⁰, —N(R¹⁰)₂, —CN, —NO₂, and C₁₋₆ alkyl optionally substituted with one or more substituents independently selected from: halogen, —OR¹⁰, —N(R¹⁰)₂, —NO₂, and —CN;
L is absent or -L¹-L²-;
   L¹ is absent;
   L² is absent or C₂₋₆ alkynylene optionally substituted with one or more substituents independently selected from: halogen, —OR¹¹, —SR¹¹, —N(R¹¹)₂, —C(O)R¹¹ —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
B is an amine-reactive lysine electrophile;
R¹ is an electrophile;
each R² is independently selected from: C₁₋₆ alkoxy;
each R³ is independently selected from:
   —OR¹³ and —N(R¹³)₂; and
   phenyl optionally substituted with one or more substituents independently selected from: halogen, —OR¹³, —SR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —P(O)(R¹³)₂, —NO₂, C₁₋₆ haloalkyl, and —CN;
R⁴ is selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy;
R^{A} and R^{B} come together to form a 4- to 10-membered heterocycle optionally substituted with one or more substituents independently selected from:
   halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
   C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
   C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN;
R¹⁰, R¹¹, R¹², R¹³, and R¹⁴ are each independently selected at each occurrence from C₁₋₆ alkyl and C₃₋₆ carbocycle, wherein the C₃₋₆ carbocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy;
n is 0;
p is 1; and
q is selected from 0 and 1.

In some embodiments, the present disclosure provides a compound represented by the structure of Formula (I):

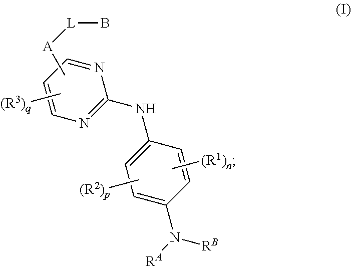

or a pharmaceutically acceptable salt thereof, wherein:
A is absent or -A¹-A²-;
   A¹ is selected from —O— and —N(R⁴)—;
   A² is phenylene;
L is absent or -L¹-L²-;
   L¹ is absent;
   L² is absent or C₂₋₆ alkynylene;
B is an amine-reactive lysine electrophile;
R¹ is an electrophile;
each R² is independently selected from: C₁₋₆ alkoxy;
each R³ is independently selected from:
   —OR¹³ and —N(R¹³)₂; and
   phenyl optionally substituted with one or more substituents independently selected from: —P(O)(R¹³)₂;
R⁴ and R⁵ are each independently selected from hydrogen and C₁₋₆ alkyl;
R^{A} and R^{B} come together to form a 4- to 10-membered heterocycle optionally substituted with one or more substituents independently selected from:
   3- to 10-membered heterocycle optionally substituted with one or more substituents independently selected from: C₁₋₆ alkyl;
R⁰, R¹¹, R¹², R¹³, and R¹⁴ are each independently selected at each occurrence from C₁₋₆ alkyl and C₃₋₆ carbocycle;
n is 0;
p is 1; and
q is selected from 0 and 1.

In some embodiments, n is 1. In some embodiments, n is 0.

In some embodiments, the amine-reactive electrophile of B is selected from aromatic aldehyde, aromatic sulfonyl fluoride, heteroaromatic aldehyde, and heteroaromatic sulfonyl fluoride. In some embodiments, the amine-reactive electrophile of B is selected from aromatic aldehyde, aromatic sulfonyl fluoride, and heteroaromatic aldehyde. In some embodiments, the amine-reactive electrophile of B is selected from aromatic aldehyde, aromatic sulfonyl fluoride, and heteroaromatic sulfonyl fluoride. In some embodiments, the amine-reactive electrophile of B is an aromatic aldehyde or aromatic sulfonyl fluoride. In some embodiments, the amine-reactive electrophile of B is an aromatic aldehyde. In some embodiments, the amine-reactive electrophile of B is an aromatic sulfonyl fluoride. In some embodiments, the amine-reactive electrophile of B is heteroaromatic aldehyde. In some embodiments, the amine-reactive electrophile of B is heteroaromatic sulfonyl fluoride.

In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from halogen, hydroxyl, C₁₋₆ alkyl, C₁₋₆ haloalkyl, C₁₋₆ alkyoxy, and C₁₋₆ haloalkyoxy. In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from halogen, C₁₋₆ alkyl, and C₁₋₆ alkyoxy. In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from halogen and C₁₋₆ alkyl. In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from halogen and C₁₋₆ alkyoxy. In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from C₁₋₆ alkyl and C₁₋₆ alkyoxy. In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from halogen. In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from C₁₋₆ alkyl. In some embodiments, the amine-reactive electrophile of B is aromatic aldehyde and the aromatic aldehyde is selected from 2-hydroxybenzaldehyde, 3-hydroxybenzaldehyde, and 4-hydroxybenzaldehyde, wherein B is optionally substituted with one or more substituent selected from C₁₋₆ alkyoxy.

In some embodiments, the aromatic aldehyde is selected from:

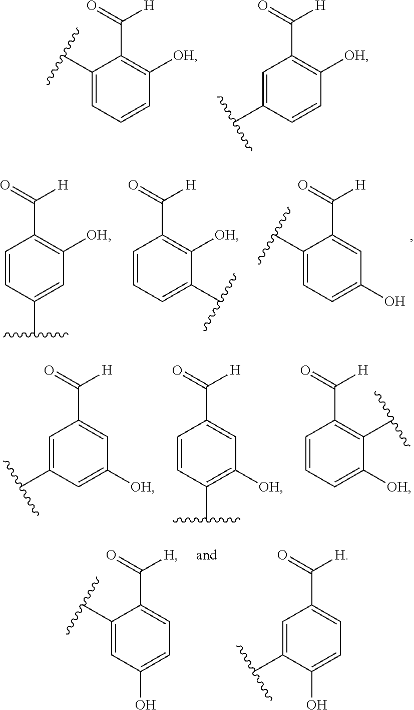

In some embodiments, the aromatic aldehyde is selected from:

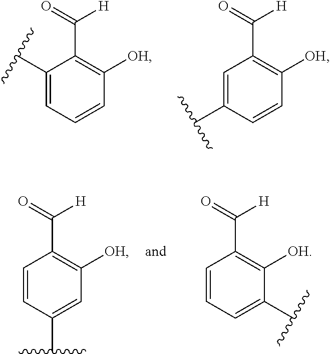 [SMILES: *c1cccc(O)c1C=O ; *c1ccc(O)c(C=O)c1 ; *c1ccc(C=O)c(O)c1 ; *c1cccc(C=O)c1O]

In some embodiments, the aromatic aldehyde is selected from:

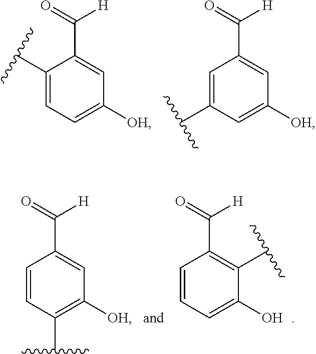 [SMILES: *c1ccc(O)cc1C=O ; *c1cc(O)cc(C=O)c1 ; *c1ccc(C=O)cc1O ; "and" ; *c1c(O)cccc1C=O]

In some embodiments, the aromatic aldehyde is selected from:

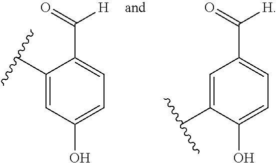

In some embodiments, the aromatic sulfonyl fluoride is benzenesulfonyl fluoride. In some embodiments, the benzenesulfonyl fluoride is selected from:

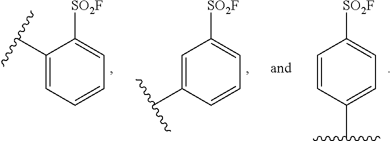 [SMILES: *c1ccccc1S(=O)(=O)F ; *c1cccc(S(=O)(=O)F)c1 ; *c1ccc(S(=O)(=O)F)cc1]

In some embodiments, the electrophile of R¹ is selected from alkenyl, alkynyl, acrylamide, acrylate, propiolamide, enone, cyanoacrylamide, haloketone, acetylenic ketone, vinyl sulfone, thiol, epoxide, nitrile, aldehyde, cycloalkyl, beta lactam, carbamoyl tetrazole, carbamoyl sulfanyl, carbamate, amino ketone, cyclipostin, dithio carbamate, ester, diazirine, sulfide, disulfide, oxime, oxime ester, phosphonate, and boronic acid. In some embodiments, the electrophile of R¹ is selected from alkenyl, alkynyl, acrylamide, acrylate, propiolamide, cyanoacrylamide, acetylenic ketone, nitrile, and vinyl sulfone.

In some embodiments, p is selected from 0, 1, 2, and 3. In some embodiments, p is selected from 0, 1, and 2. In some embodiments, p is selected from 1, 2, and 3. In some embodiments, p is selected from 1 and 2. In some embodiments, p is 0. In some embodiments, p is 1. In some embodiments, p is 2. In some embodiments, p is 3.

In some embodiments, each R² is independently selected from halogen, C₁₋₆ haloalkyl, C₁₋₆ hydroxyalkyl, C₁₋₆ alkoxy, C₁₋₆ haloalkoxy, —OR¹², —SR¹², —N(R¹²)₂, —C(O)OR¹², —OC(O)R¹², —C(O)N(R¹²)₂, —N(R¹²)C(O)R¹², —CN, and —NO₂. In some embodiments, each R² is independently selected from C₁₋₆ haloalkyl, C₁₋₆ hydroxyalkyl, C₁₋₆ alkoxy, C₁₋₆ haloalkoxy, and —N(R¹²)C(O)R¹². In some embodiments, each R² is independently selected from C₁₋₆ haloalkyl, C₁₋₆ hydroxyalkyl, C₁₋₆ alkoxy, and C₁₋₆ haloalkoxy. In some embodiments, each R² is selected from C₁₋₆ alkoxy and —N(R¹²)C(O)R¹². In some embodiments, each R² is C₁₋₆ alkoxy. In some embodiments, each R² is —N(R¹²)C(O)R¹².

In some embodiments, q is selected from 0, 1, and 2. In some embodiments, q is selected from 0 and 1. In some embodiments, q is 0. In some embodiments, q is 1.

In some embodiments, R³ is selected from:
halogen, —OR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —NO₂, and —CN; and
C₁₋₆ alkyl, C₂₋₆ alkenyl, monocyclic 5- to 6-membered heteroaryl, bicyclic heteroaryl, bicyclic aryl, and phenyl, each of which is optionally substituted with one or more substituents independently selected from: halogen, —OR¹³, —N(R¹³)₂, —C(O)R¹³, —C(O)OR¹³ —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)₂N(R¹³)₂, —P(O)(R¹³)₂, —NO₂, C₁₋₆ alkyl, C₁₋₆ haloalkyl, and —CN.

In some embodiments, R³ is selected from:
halogen, —OR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, and —N(R¹³)S(O)₂(R¹³); and
C₁₋₆ alkyl, C₂₋₆ alkenyl, monocyclic 5- to 6-membered heteroaryl, bicyclic heteroaryl, bicyclic aryl, and phenyl, each of which is optionally substituted with one or more substituents independently selected from: halogen, —OR¹³, —N(R¹³)₂, —C(O)R¹³, —C(O)OR¹³ —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)₂N(R¹³)₂, —P(O)(R¹³)₂, C₁₋₆ alkyl, and C₁₋₆ haloalkyl.

In some embodiments, R³ is selected from: halogen, —OR¹³, —N(R¹³)₂, and —N(R¹³)C(O)R¹³; and bicyclic heteroaryl and phenyl, each of which is optionally substituted with one or more substituents independently selected from: —P(O)(R¹³)₂ and C₁₋₆ alkyl. In some embodiments, R³ is selected from: halogen and —N(R¹³)C(O)R¹³; and bicyclic heteroaryl optionally substituted with one or more substituents independently selected from: C₁₋₆ alkyl. In some embodiments, R³ is selected from: —OR¹³ and —N(R¹³)₂; and phenyl optionally substituted with one or more substituents independently selected from: —P(O)(R¹³)₂.

In some embodiments, R⁴ and R⁵ are each independently selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy. In some embodiments, R⁴ and R⁵ are each independently selected from hydrogen and C₁₋₆ alkyl. In some embodiments, R⁴ and R⁵ are each independently selected from C₁₋₆ alkyl optionally substituted with one or more substituents independently selected from: halogen, —OH, —NH₂, —NO₂, —CN, C₁₋₆ alkoxy, and C₁₋₆ haloalkoxy. In some embodiments, R⁴ and R⁵ are each independently selected from Cl-6 alkyl. In some embodiments, R⁴ and R⁵ are each hydrogen. In some embodiments, R⁴ is selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy. In some embodiments, R⁴ is selected from hydrogen and C₁₋₆ alkyl. In some embodiments, R⁴ is C₁₋₆ alkyl optionally substituted with one or more substituents independently selected from: halogen, —OH, —NH₂, —NO₂, —CN, C₁₋₆ alkoxy, and C₁₋₆ haloalkoxy. In some embodiments, R⁴ is C₁₋₆ alkyl. In some embodiments, R⁴ is hydrogen. In some embodiments, R⁵ is selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy. In some embodiments, R⁵ is selected from hydrogen and C₁₋₆ alkyl. In some embodiments, R⁵ is C₁₋₆ alkyl optionally substituted with one or more substituents independently selected from: halogen, —OH, —NH₂, —NO₂, —CN, C₁₋₆ alkoxy, and Cl-6 haloalkoxy. In some embodiments, R⁵ is C₁₋₆ alkyl. In some embodiments, R⁵ is hydrogen.

In some embodiments, R^{A} and R^{B} are each independently selected from:
hydrogen; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴ —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴ —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; or
R^{A} and R^{B} come together to form a 4- to 10-membered heterocycle optionally substituted with one or more substituents independently selected from:
   halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
   C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
   C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN.

In some embodiments, R^{A} and R^{B} are each independently selected from:
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; or
   R^{A} and R^{B} come together to form a 4- to 10-membered heterocycle optionally substituted with one or more substituents independently selected from:
C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴ —N(R¹⁴)₂, —C(O)R¹⁴, —C(O)OR¹⁴, —NO₂, and —CN.

In some embodiments, R^{A} and R^{B} are each independently selected from:
C₁₋₆ alkyl optionally substituted with one or more substituents independently selected from: —N(R¹⁴)₂; or
   R^{A} and R^{B} come together to form a 4- to 10-membered heterocycle optionally substituted with one or more substituents independently selected from:
3- to 10-membered heterocycle optionally substituted with one or more substituents independently selected from: halogen, —OR¹⁴, —SR¹⁴, —N(R¹⁴)₂, —C(O)R¹⁴ —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴ —SR¹⁴, —N(R¹⁴)₂, —C(O)R¹⁴, —C(O)OR¹⁴, —NO₂, and —CN.

In some embodiments, R^{A} and R^{B} are each independently selected from:
C₁₋₆ alkyl optionally substituted with one or more substituents independently selected from: —N(R¹⁴)₂; or
   R^{A} and R^{B} come together to form a 4- to 10-membered heterocycle optionally substituted with one or more substituents independently selected from:
3- to 10-membered heterocycle optionally substituted with one or more substituents independently selected from: C₁₋₆ alkyl optionally substituted with one or more substituents independently selected from: halogen, —OR¹⁴ —SR¹⁴, —N(R¹⁴)₂, —C(O)R¹⁴, —C(O)OR¹⁴, —NO₂, and —CN.

In some embodiments, R^{A} and R^{B} are each independently selected from:
C₁₋₆ alkyl optionally substituted with one or more substituents independently selected from: —N(R¹⁴)₂; or
   R^{A} and R^{B} come together to form a 4- to 10-membered heterocycle optionally substituted with one or more substituents independently selected from:
3- to 10-membered heterocycle optionally substituted with one or more substituents independently selected from: C₁₋₆ alkyl.

In some embodiments, R^{A} and R^{B} are each independently selected from hydrogen and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN. In some embodiments, R^{A} and R^{B} are each independently selected from hydrogen and C₁₋₆ alkyl optionally substituted with one or more substituents independently selected from: halogen, —OR¹⁴, —N(R¹⁴)₂, —C(O)R¹⁴, —C(O)OR¹⁴ —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —NO₂, and —CN. In some embodiments, R^{A} and R^{B} are each independently selected from hydrogen and C₁₋₆ alkyl optionally substituted with one or more substituents independently selected from: halogen, —OR¹⁴, —N(R¹⁴)₂, —C(O)R¹⁴, —C(O)OR¹⁴ —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, and —N(R¹⁴)S(O)₂(R¹⁴). In some embodiments, R^{A} and R^{B} are each independently selected from hydrogen and C₁₋₆ alkyl optionally substituted with one or more substituents independently selected from: halogen, —OR¹⁴, —N(R¹⁴)₂, —C(O)R¹⁴, —C(O)OR¹⁴ —NO₂, and —CN. In some embodiments, R^{A} and R^{B} are each independently selected from hydrogen and C₁₋₆ alkyl optionally substituted with one or more substituents independently selected from: —OR¹⁴ and —N(R¹⁴)₂. In some embodiments, R^{A} and R^{B} are each independently selected from hydrogen and C₁₋₆ alkyl optionally substituted with one or more substituents independently selected from —N(R¹⁴)₂. In some embodiments, R^{A} and R^{B} are each independently selected from: C₁₋₆ alkyl optionally substituted with one or more substituents independently selected from: —N(R¹⁴)₂. In some embodiments, R^{A} and R^{B} are each hydrogen.

In some embodiments, R^{A} is selected from: hydrogen; and C₁₋₆ alkyl optionally substituted with one or more substituents independently selected from: halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN. In some embodiments, R^{A} is selected from: hydrogen; and C₁₋₆ alkyl optionally substituted with one or more substituents independently selected from: halogen, —OR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —NO₂, and —CN. In some embodiments, R^{A} is selected from: hydrogen; and C₁₋₆ alkyl optionally substituted with one or more substituents independently selected from: halogen, —OR¹⁴, —N(R¹⁴)₂, —C(O)R¹⁴, —C(O)OR¹⁴ —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, and —N(R¹⁴)S(O)₂(R¹⁴). In some embodiments, R^{A} is selected from hydrogen and C₁₋₆ alkyl optionally substituted with one or more substituents independently selected from: halogen, —OR¹⁴, —N(R¹⁴)₂, —C(O)R¹⁴, —C(O)OR¹⁴, —NO₂, and —CN. In some embodiments, R^{A} is selected from hydrogen and C₁₋₆ alkyl optionally substituted with one or more substituents independently selected from: —OR¹⁴ and —N(R¹⁴)₂. In some embodiments, R^{A} is selected from hydrogen and C₁₋₆ alkyl optionally substituted with one or more substituents independently selected from —N(R¹⁴)₂. In some embodiments, R^{A} is selected from C₁₋₆ alkyl optionally substituted with one or more substituents independently selected from —N(R¹⁴)₂. In some embodiments, R^{A} is hydrogen.

In some embodiments, R^{B} is selected from: hydrogen; and C₁₋₆ alkyl optionally substituted with one or more substituents independently selected from: halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN. In some embodiments, R^{B} is selected from: hydrogen; and C₁₋₆ alkyl optionally substituted with one or more substituents independently selected from: halogen, —OR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —NO₂, and —CN. In some embodiments, R^{B} is selected from: hydrogen; and C₁₋₆ alkyl optionally substituted with one or more substituents independently selected from: halogen, —OR¹⁴, —N(R¹⁴)₂, —C(O)R¹⁴, —C(O)OR¹⁴ —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, and —N(R¹⁴)S(O)₂(R¹⁴). In some embodiments, R^{B} is selected from hydrogen and C₁₋₆ alkyl optionally substituted with one or more substituents independently selected from: halogen, —OR¹⁴, —N(R¹⁴)₂, —C(O)R¹⁴, —C(O)OR¹⁴, —NO₂, and —CN. In some embodiments, R^{B} is selected from hydrogen and C₁₋₆ alkyl optionally substituted with one or more substituents independently selected from: —OR¹⁴ and —N(R¹⁴)₂. In some embodiments, R^{B} is selected from hydrogen and C₁₋₆ alkyl optionally substituted with one or more substituents independently selected from —N(R¹⁴)₂. In some embodiments, R^{B} is C₁₋₆ alkyl optionally substituted with one or more substituents independently selected from —N(R¹⁴)₂. In some embodiments, R^{B} is hydrogen.

In some embodiments, R^{A} and R^{B} come together to form a 4- to 10-membered heterocycle optionally substituted with one or more substituents independently selected from:
halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN.

In some embodiments, R^{A} and R^{B} come together to form a 4- to 10-membered heterocycle optionally substituted with one or more substituents independently selected from:
halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN.

In some embodiments, R^{A} and R^{B} come together to form a 4- to 10-membered heterocycle optionally substituted with one or more substituents independently selected from:
halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —NO₂, and —CN; and
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —NO₂, and —CN.

In some embodiments, R^{A} and R^{B} come together to form a 4- to 10-membered heterocycle optionally substituted with one or more substituents independently selected from:
halogen, —OR¹⁴, —SR¹⁴ —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —NO₂, and —CN; and
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, and —N(R¹⁴)S(O)₂(R¹⁴).

In some embodiments, R^{A} and R^{B} come together to form a 4- to 10-membered heterocycle optionally substituted with one or more substituents independently selected from: halogen, —OR¹⁴, —N(R¹⁴)₂, —C(O)R¹⁴, —C(O)OR¹⁴, —NO₂, and —CN; and C₁₋₆ alkyl optionally substituted with one or more substituents independently selected from: halogen, —OR¹⁴ —N(R¹⁴)₂, —C(O)R¹⁴, —C(O)OR¹⁴, —NO₂, and —CN.

In some embodiments, R^{A} and R^{B} come together to form a 4- to 10-membered heterocycle optionally substituted with one or more substituents independently selected from:
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN.

In some embodiments, R^{A} and R^{B} come together to form a 4- to 10-membered heterocycle optionally substituted with one or more substituents independently selected from:
halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴ —N(R¹⁴)₂, —C(O)R¹⁴, —C(O)OR¹⁴, —NO₂, and —CN.

In some embodiments, R^{A} and R^{B} come together to form a 4- to 10-membered heterocycle optionally substituted with one or more substituents independently selected from:
C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN.

In some embodiments, R^{A} and R^{B} come together to form a 4- to 10-membered heterocycle optionally substituted with one or more substituents independently selected from:
C₃₋₁₀ carbocycle and 3- to 10-membered heterocycle, any of which is optionally substituted with one or more substituents independently selected from: halogen, —OR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —NO₂, —CN; and C₁₋₆ alkyl optionally substituted with one or more substituents independently selected from: halogen, —OR¹⁴, —N(R¹⁴)₂, —C(O)R¹⁴, —C(O)OR¹⁴, —NO₂, and —CN.

In some embodiments, R^{A} and R^{B} come together to form a 4- to 10-membered heterocycle optionally substituted with one or more substituents independently selected from:
C₃₋₁₀ carbocycle and 3- to 10-membered heterocycle, any of which is optionally substituted with one or more substituents independently selected from: halogen, —OR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, and —N(R¹⁴)S(O)₂(R¹⁴); and C₁₋₆ alkyl optionally substituted with one or more substituents independently selected from: halogen, —OR¹⁴, —N(R¹⁴)₂, —C(O)R¹⁴ and —C(O)OR¹⁴.

In some embodiments, R^{A} and R^{B} come together to form a 4- to 10-membered heterocycle optionally substituted with one or more substituents independently selected from:
C₃₋₁₀ carbocycle and 3- to 10-membered heterocycle, any of which is optionally substituted with one or more substituents independently selected from: C₁₋₆ alkyl optionally substituted with one or more substituents independently selected from: halogen, —OR¹¹, —N(R¹⁴)₂, —C(O)R¹⁴, and —C(O)OR¹⁴.

In some embodiments, R^{A} and R^{B} come together to form a 4- to 10-membered heterocycle optionally substituted with one or more substituents independently selected from:
C₃₋₁₀ carbocycle and 3- to 10-membered heterocycle, any of which is optionally substituted with one or more substituents independently selected from: C₁₋₆ alkyl.

In some embodiments, R^{A} and R^{B} come together to form a 4- to 10-membered heterocycle optionally substituted with one or more substituents independently selected from:
3- to 10-membered heterocycle, any of which is optionally substituted with one or more substituents independently selected from: C₁₋₆ alkyl.

In some embodiments, A² selected from a monocyclic 5- to 6-membered heteroarylene, bicyclic heteroarylene, bicyclic arylene, and phenylene, any of which are optionally substituted with one or more substituents selected from: halogen, —OR¹⁰, —N(R¹⁰)₂, —CN, —NO₂, and C₁₋₆ alkyl optionally substituted with one or more substituents independently selected from: halogen, —OR¹⁰, —N(R¹⁰)₂, —NO₂, and —CN. In some embodiments, A² selected from a monocyclic 5- to 6-membered heteroarylene and bicyclic heteroarylene, any of which are optionally substituted with one or more substituents selected from: halogen, —OR¹⁰, —N(R¹⁰)₂, —CN, —NO₂, and C₁₋₆ alkyl optionally substituted with one or more substituents independently selected from: halogen, —OR¹⁰, —N(R¹⁰)₂, —NO₂, and —CN. In some embodiments, A² selected from a monocyclic 5- to 6-membered heteroarylene and bicyclic heteroarylene, any of which are optionally substituted with one or more substituents selected from: halogen, —NH₂, —NO₂; and C₁₋₆ alkyl optionally substituted with one or more substituents independently selected from: halogen, —OR¹⁰, —N(R¹⁰)₂, —NO₂, and —CN. In some embodiments, A² is a monocyclic 5- to 6-membered heteroarylene optionally substituted with one or more substituents selected from: halogen, —NH₂, —NO₂; and C₁₋₆ alkyl optionally substituted with one or more substituents independently selected from: halogen, —OR¹⁰, —N(R¹⁰)₂, —NO₂, and —CN. In some embodiments, A² is a monocyclic 5- to 6-membered heteroaryl selected from pyrrole, pyrazole, imidazole, triazole, tetrazole, oxazole, isoxazole, isothiazole, thiazole, oxadiazole, thiadiazole, pyridine, pyridazine, pyrimidine, pyrazine, and triazine, any of which is optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN. In some embodiments, A² is pyrazole optionally substituted with C₁₋₆ alkyl optionally substituted with one or more substituents independently selected from: halogen, —OR¹⁰, —N(R¹⁰)₂, —NO₂, and —CN; and R¹⁰ is selected from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, A² is bicyclic heteroarylene optionally substituted with one or more substituents selected from: halogen, —NH₂, —NO₂; and C₁₋₆ alkyl optionally substituted with one or more substituents independently selected from: halogen, —OR¹⁰, —N(R¹⁰)₂, —NO₂, and —CN. In some embodiments, A² is a bicyclic heteroaryl selected from indole, isoindole, indolizine, indazole, benzimidazole, azaindole, purine, benzisoxazole, benzoxazole, benzisothiazole, benzoisothiazole, imidazopyridine, thiazolepyridine, and oxazolepyridine, any one of which is optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN. In some embodiments, the bicyclic heteroaryl of A² is optionally substituted indole.

In some embodiments, L is absent or -L¹-L²-, wherein:
L¹ is absent or selected from —O—, —N(R⁵)—, and —S—; and
L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN.

In some embodiments, L is absent or -L¹-L²-, wherein:
L¹ is absent or selected from —O— and —N(R⁵)—; and
   L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R^{11)S(O)}₂(R¹¹), —NO₂, and —CN.

In some embodiments, L is absent or -L¹-L²-, wherein:
L¹ is absent or selected from —O— and —N(R⁵)—; and
   L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R^{11)S(O)}₂(R¹¹).

In some embodiments, L is absent or -L¹-L²-, wherein:
L¹ is absent; and
   L² is absent or selected from C₁₋₆ alkylene and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R^{11)S(O)}₂(R¹¹).

In some embodiments, L is absent or -L¹-L²-, wherein:
L¹ is absent; and
L² is absent or selected from C₁₋₆ alkylene and C₂₋₆ alkynylene.

In some embodiments, L is absent.

In some embodiments, L is -L¹-L²-, wherein:
L¹ is absent or selected from —O—, —N(R⁵)—, and —S—; and
L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN.

In some embodiments, L is -L¹-L²-, wherein:
L¹ is absent or selected from —O— and —N(R⁵)—; and
L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —NO₂, and —CN.

In some embodiments, L is -L¹-L²-, wherein:
L¹ is absent or selected from —O— and —N(R⁵)—; and
L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R^{11)S(O)}₂(R¹¹).

In some embodiments, L is -L¹-L²-, wherein:
L¹ is absent; and
L² is absent or selected from C₁₋₆ alkylene and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R^{11)S(O)}₂(R¹¹).

In some embodiments, L is -L¹-L²-, wherein:
L¹ is absent; and
L² is absent or selected from C₁₋₆ alkylene and C₂₋₆ alkynylene.

In some embodiments, L¹ is absent or selected from —O—, —N(R⁵)—, and —S—. In some embodiments, L¹ is absent or selected from —O— and —N(R⁵)—. In some embodiments, L¹ is absent or selected from —O— and —NH—. In some embodiments, L¹ is absent or —O—. In some embodiments, L¹ is absent or —N(R⁵)—. In some embodiments, L¹ is absent or —NH—. In some embodiments, L¹ is —O—. In some embodiments, L¹ is —N(R⁵)—. In some embodiments, L¹ is —NH—. In some embodiments, L¹ is absent.

In some embodiments, L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹ —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R^{11)S(O)}₂(R¹¹), —S(O)R¹¹, —S(O)₂R¹¹, —S(O)₂N(R¹¹)₂, —NO₂, and —CN. In some embodiments, L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹, —OC(O)R¹¹ —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —NO₂, and —CN. In some embodiments, L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹ —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R¹¹)S(O)₂(R¹¹). In some embodiments, L² is absent or selected from C₁₋₆ alkylene and C₂₋₆ alkynylene, each of which is optionally substituted with one or more substituents independently selected from: halogen, —OR¹¹, —SR¹¹, —N(R¹¹)₂, —C(O)R¹¹ —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R¹¹)S(O)₂(R¹¹). In some embodiments, L² is absent or C₁₋₆ alkylene optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R¹¹)S(O)₂(R¹¹). In some embodiments, L² is absent or C₁₋₆ alkylene. In some embodiments, L² C₁₋₆ alkylene. In some embodiments, L² is absent or C₂₋₆ alkynylene, each of which is optionally substituted with one or more substituents independently selected from: halogen, —OR¹¹, —SR¹¹, —N(R¹¹)₂, —C(O)R¹¹ —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, and —N(R¹¹)S(O)₂(R¹¹). In some embodiments, L² is absent or C₂₋₆ alkynylene. In some embodiments, L² is C₂₋₆ alkynylene. In some embodiments, L² is absent.

In some embodiments, R¹⁰, R¹¹, R¹², R¹³, and R¹⁴ are each independently selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁰, R¹¹, R¹², R¹³, and R¹⁴ are each independently selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁰, R¹¹, R¹², R¹³, and R¹⁴ are each independently selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle. In some embodiments, R¹⁰, R¹¹, R¹², R¹³, and R¹⁴ are each independently selected at each occurrence from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁰, R¹¹, R¹², R¹³, and R¹⁴ are each independently selected at each occurrence from hydrogen, C₁₋₆ alkyl, and C₃₋₆ carbocycle. In some embodiments, R¹⁰, R¹¹, R¹², R¹³, and R¹⁴ are each independently selected at each occurrence from hydrogen and C₁₋₆ alkyl. In some embodiments, R¹⁰, R¹¹, R¹², R¹³, and R¹⁴ are each independently selected at each occurrence from hydrogen and C₃₋₆ carbocycle. In some embodiments, R¹⁰, R¹¹, R¹², R¹³, and R¹⁴ are each independently selected at each occurrence from C₁₋₆ alkyl and C₃₋₆ carbocycle. In some embodiments, R¹⁰, R⁸¹, R¹², R¹³, and R¹⁴ are each hydrogen. In some embodiments, R¹⁰, R¹¹, R¹², R¹³, and R¹⁴ are each independently C₁₋₆ alkyl. In some embodiments, R¹⁰, R¹¹, R¹², R¹³, and R¹⁴ are each independently C₃₋₆ carbocycle.

In some embodiments, R¹⁰ is selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁰ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁰ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle. In some embodiments, R¹⁰ is selected at each occurrence from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁰ is selected at each occurrence from hydrogen, C₁₋₆ alkyl, and C₃₋₆ carbocycle. In some embodiments, R¹⁰ is selected at each occurrence from hydrogen and C₁₋₆ alkyl. In some embodiments, R¹⁰ is selected at each occurrence from hydrogen and C₃₋₆ carbocycle. In some embodiments, R¹⁰ is selected at each occurrence from C₁₋₆ alkyl and C₃₋₆ carbocycle. In some embodiments, R¹⁰ is hydrogen. In some embodiments, R¹⁰ is C₁₋₆ alkyl. In some embodiments, R¹⁰ is C₃₋₆ carbocycle.

In some embodiments, R¹¹ is selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹¹ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹¹ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle. In some embodiments, R¹¹ is selected at each occurrence from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R¹¹ is selected at each occurrence from hydrogen, C₁₋₆ alkyl, and C₃₋₆ carbocycle. In some embodiments, R¹¹ is selected at each occurrence from hydrogen and C₁₋₆ alkyl. In some embodiments, R¹¹ is selected at each occurrence from hydrogen and C₃₋₆ carbocycle. In some embodiments, R¹¹ is selected at each occurrence from C₁₋₆ alkyl and C₃₋₆ carbocycle. In some embodiments, R¹¹ is hydrogen. In some embodiments, R¹¹ is C₁₋₆ alkyl. In some embodiments, R¹¹ is C₃₋₆ carbocycle.

In some embodiments, R¹² is selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹² is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹² is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle. In some embodiments, R¹² is selected at each occurrence from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R¹² is selected at each occurrence from hydrogen, C₁₋₆ alkyl, and C₃₋₆ carbocycle. In some embodiments, R¹² is selected at each occurrence from hydrogen and C₁₋₆ alkyl. In some embodiments, R¹² is selected at each occurrence from hydrogen and C₃₋₆ carbocycle. In some embodiments, R¹² is selected at each occurrence from C₁₋₆ alkyl and C₃₋₆ carbocycle. In some embodiments, R¹² is hydrogen. In some embodiments, R¹² is C₁₋₆ alkyl. In some embodiments, R¹² is C₃₋₆ carbocycle.

In some embodiments, R¹³ is selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹³ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹³ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle. In some embodiments, R¹³ is selected at each occurrence from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R¹³ is selected at each occurrence from hydrogen, C₁₋₆ alkyl, and C₃₋₆ carbocycle. In some embodiments, R¹³ is selected at each occurrence from hydrogen and C₁₋₆ alkyl. In some embodiments, R¹³ is selected at each occurrence from hydrogen and C₃₋₆ carbocycle. In some embodiments, R¹³ is selected at each occurrence from C₁₋₆ alkyl and C₃₋₆ carbocycle. In some embodiments, R¹³ is hydrogen. In some embodiments, R¹³ is C₁₋₆ alkyl. In some embodiments, R¹³ is C₃₋₆ carbocycle.

In some embodiments, R¹⁴ is selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁴ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁴ is selected at each occurrence from hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle. In some embodiments, R¹⁴ is selected at each occurrence from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R¹⁴ is selected at each occurrence from hydrogen, C₁₋₆ alkyl, and C₃₋₆ carbocycle. In some embodiments, R¹⁴ is selected at each occurrence from hydrogen and C₁₋₆ alkyl. In some embodiments, R¹⁴ is selected at each occurrence from hydrogen and C₃₋₆ carbocycle. In some embodiments, R¹⁴ is selected at each occurrence from C₁₋₆ alkyl and C₃₋₆ carbocycle. In some embodiments, R¹⁴ is hydrogen. In some embodiments, R¹⁴ is C₁₋₆ alkyl. In some embodiments, R¹⁴ is C₃₋₆ carbocycle.

In some aspects, the present disclosure provides compounds represented by the structure of Formula (I):

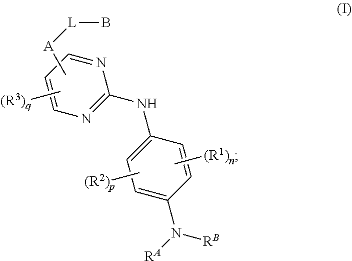

or a pharmaceutically acceptable salt thereof, wherein:
A is absent or selected from a monocyclic 5- to 6-membered heteroaryl, bicyclic heteroaryl, bicyclic aryl, and phenyl, any of which are optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN;
L is selected from: a bond, —O—, N(R¹⁵), S(R¹⁶); and C₁₋₆ alkylene optionally substituted with one or more substituents independently selected from: halogen, —OR¹¹, —SR¹¹ —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹ —S(O)₂N(R¹¹)₂, —NO₂, and —CN;
B is an amine-reactive lysine electrophile;
R¹ is an electrophile;
each R² is independently selected from: halogen, C₁₋₆ haloalkyl, C₁₋₆ hydroxyalkyl, C₁₋₆ alkoxy, C₁₋₆ haloalkoxy, —CN, —NO₂, and —NH₂;
each R³ is independently selected from:
   halogen, —OR¹², —SR¹² —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹², —S(O)₂N(R¹²)₂, —NO₂, and —CN; and
   C₁₋₆ alkyl C₂₋₆ alkenyl monocyclic 5- to 6-membered heteroaryl, bicyclic heteroaryl, bicyclic aryl, and phenyl, each of which is optionally substituted with one or more substituents independently selected from: halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹² —S(O)₂N(R¹²)₂, —NO₂, C₁₋₆ alkyl, C₁₋₆ haloalkyl, and —CN;
R^{A} and R^{B} are each independently selected from hydrogen; and C₁₋₆ alkyl optionally substituted with one or more substituents independently selected from: halogen, —OR¹³, —SR¹³ —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —NO₂, and —CN; or
R^{A} and R^{B} can come together to form a 4- to 10-membered heterocycle optionally substituted with one or more substituents independently selected from:
   halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
   C₁₋₆ alkyl optionally substituted with one or more substituents independently selected from: halogen, —OR¹⁴, —SR¹⁴, —N(R¹⁴)₂, —C(O)R¹⁴ —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
   C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴ C(O)OR¹⁴, —NO₂, and —CN;
R¹⁰, R¹¹, R¹², R¹³, R¹⁴, R¹⁵, and R¹⁶ are each independently selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy;
n is selected from 0 and 1;
p is selected from 0, 1, 2, and 3; and
q is selected from 0, 1, and 2.

In some embodiments, the amine-reactive electrophile of B is selected from aromatic aldehyde, aromatic sulfonyl fluoride, heteroaromatic aldehyde, and heteroaromatic sulfonyl fluoride. In some embodiments, the amine-reactive electrophile of B is selected from aromatic aldehyde, aromatic sulfonyl fluoride, heteroaromatic aldehyde, and heteroaromatic sulfonyl fluoride. In some embodiments, the amine-reactive electrophile of B is an aromatic aldehyde. In some embodiments, the amine-reactive electrophile of B an aromatic sulfonyl fluoride. In some embodiments, the amine-reactive electrophile of B is a heteroaromatic aldehyde. In some embodiments, the amine-reactive electrophile of B is a heteroaromatic sulfonyl fluoride. In some embodiments, the amine-reactive electrophile of B is a heteroaromatic aldehyde or a heteroaromatic sulfonyl fluoride, wherein the heteroaromatic group of each comprises at least one nitrogen atom, oxygen atom, sulfur atom, or any combination thereof. In some embodiments, the amine-reactive electrophile of B is aromatic aldehyde or aromatic sulfonyl fluoride.

In some embodiments, the amine-reactive electrophile of B is a reversible covalent electrophile. In some embodiments, the reversible covalent electrophile of B is an aromatic aldehyde.

In some embodiments, the amine-reactive electrophile of B is an aromatic aldehyde selected from 2-hydroxybenzaldehyde, 3-hydroxybenzaldehyde, and 4-hydroxybenzaldehyde.

In some embodiments, the reversible covalent electrophile is aromatic aldehyde is selected from 2-hydroxybenzaldehyde, 3-hydroxybenzaldehyde, and 4-hydroxybenzaldehyde. In some embodiments, the amine-reactive electrophile of B is an aromatic aldehyde is selected from:

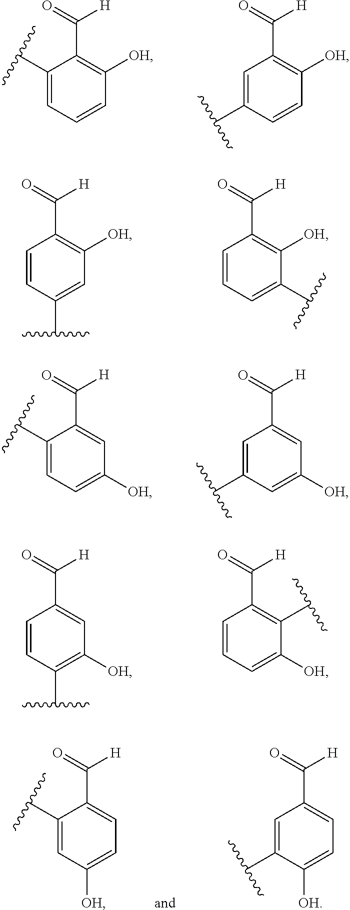

In some embodiments, the amine-reactive electrophile of B is an aromatic aldehyde is selected from:

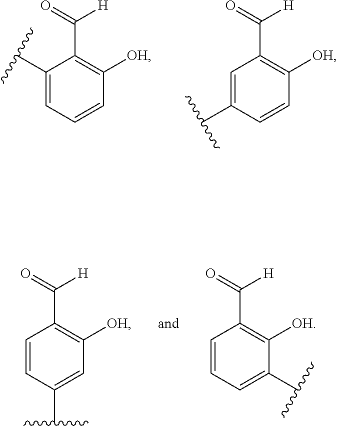 [SMILES: *c1cccc(O)c1C=O ; *c1ccc(O)c(C=O)c1 ; *c1ccc(C=O)c(O)c1 ; "and" ; *c1cccc(C=O)c1O]

In some embodiments, the amine-reactive electrophile of B is an aromatic aldehyde is selected from:

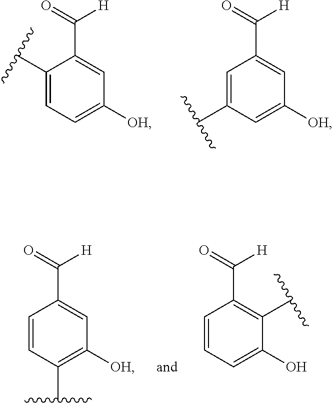 [SMILES: *c1ccc(O)cc1C=O ; *c1cc(O)cc(C=O)c1 ; *c1ccc(C=O)cc1O ; "and" ; *c1c(O)cccc1C=O]

In some embodiments, the amine-reactive electrophile of B is an aromatic aldehyde is selected from:

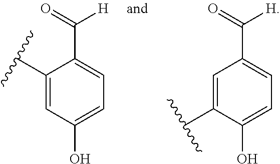

In some embodiments, the amine-reactive electrophile of B is benzenesulfonyl fluoride. In some embodiments, the amine-reactive electrophile of B is a benzenesulfonyl fluoride is selected from

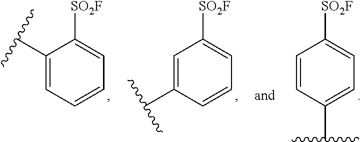

In some embodiments, the amine-reactive electrophile of B is

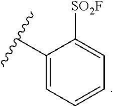 [SMILES: *c1ccccc1S(=O)(=O)F]

In some embodiments, the amine-reactive electrophile of B is

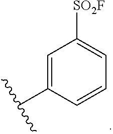 [SMILES: *c1cccc(S(=O)(=O)F)c1]

In some embodiments, the amine-reactive electrophile of B is

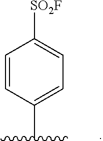 [SMILES: *c1ccc(S(=O)(=O)F)cc1 ; "."]

In some embodiments, n is 0. In some embodiments, n is 1.

In some embodiments, the electrophile of R¹ is selected from alkenyl, alkynyl, acrylamide, acrylate, propiolamide, enone, cyanoacrylamide, haloketone, acetylenic ketone, vinyl sulfone, thiol, epoxide, nitrile, aldehyde, cycloalkyl, beta lactam, carbamoyl tetrazole, carbamoyl sulfanyl, carbamate, amino ketone, cyclipostin, dithio carbamate, ester, diazirine, sulfide, disulfide, oxime, oxime ester, phosphonate, and boronic acid. In some embodiments, the electrophile of R¹ is selected from alkenyl, alkynyl, acrylamide, acrylate, propiolamide, cyanoacrylamide, acetylenic ketone, nitrile, and vinyl sulfone. In some embodiments, the electrophile of R¹ is selected alkenyl, alkynyl, acrylamide, propiolamide, and vinyl sulfone. In some embodiments, the electrophile of R¹ is selected acrylamide and propiolamide. In some embodiments, the electrophile of R¹ is an acrylamide.

In some embodiments, p is 0. In some embodiments, p is 1. In some embodiments, p is 2. In some embodiments, p is 3. In some embodiments, p is selected from 0, 1, and 2. In some embodiments, p is selected from 0 and 1. In some embodiments, p is selected from 1, 2, and 3. In some embodiments, p is selected from 1 and 2. In some embodiments, p is selected from 2 and 3.

In some embodiments, R² is selected from C₁₋₆ haloalkyl, C₁₋₆ hydroxyalkyl, C₁₋₆ alkoxy, and C₁₋₆ haloalkoxy. In some embodiments, R² is selected from —CN, —NO₂, and —NH₂. In some embodiments, R² is selected from C₁₋₆ alkoxy and C₁₋₆ hydroxyalkyl. In some embodiments, R² is selected from C₁₋₆ alkoxy. In some embodiments, R² is selected from C₁₋₆ hydroxyalkyl.

In some embodiments, q is 0. In some embodiments, q is 1.

In some embodiments, R³ is selected from halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹², —S(O)₂N(R¹²)₂, —NO₂, and —CN. In some embodiments, R³ is selected from —OR¹², —C(O)R¹², —C(O)OR¹², —OC(O)R¹², —OC(O)N(R¹²)₂, and —C(O)N(R¹²)₂. In some embodiments, R³ is selected from —C(O)R¹² and —C(O)OR¹². In some embodiments, R³ is —C(O)R¹². In some embodiments, R³ is —C(O)OR¹²

In some embodiments, R³ is selected from C₁₋₆ alkyl, C₂₋₆ alkenyl, monocyclic 5- to 6-membered heteroaryl, bicyclic heteroaryl, bicyclic aryl, and phenyl, each of which is optionally substituted with one or more substituents independently selected from halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹², —S(O)₂N(R¹²)₂, —NO₂, —CN, C₁₋₆ alkyl and C₁₋₆ haloalkyl. In some embodiments, R³ is selected from C₁₋₆ alkyl and C₂₋₆ alkenyl each of which is optionally substituted with one or more substituents independently selected from halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹², —S(O)₂N(R¹²)₂, —NO₂, and —CN. In some embodiments, R³ is selected from monocyclic 5- to 6-membered heteroaryl, bicyclic heteroaryl, bicyclic aryl, and phenyl, each of which is optionally substituted with one or more substituents independently selected from halogen, —OR¹², —SR¹², —N(R¹²)₂, —C(O)R¹², —C(O)OR¹², —OC(O)R¹², —OC(O)N(R¹²)₂, —C(O)N(R¹²)₂, —N(R¹²)C(O)R¹², —N(R¹²)C(O)OR¹², —N(R¹²)C(O)N(R¹²)₂, —N(R¹²)S(O)₂(R¹²), —S(O)R¹², —S(O)₂R¹², —S(O)₂N(R¹²)₂, —NO₂, —CN, C₁₋₆ alkyl and C₁₋₆ haloalkyl.

In some embodiments, R^{A} is selected from hydrogen and C₁₋₆ alkyl optionally substituted with one or more substituents independently selected from: halogen, —OR¹³, and —N(R¹³)₂; and R¹³ is selected from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R^{A} is C₁₋₆ alkyl optionally substituted with one or more substituents independently selected from: halogen, —OR¹³, and —N(R¹³)₂; and R¹³ is selected from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R^{A} is selected from C₁₋₆ alkyl optionally substituted with halogen, —OR¹³, and —N(R¹³)₂; and R¹³ is selected from hydrogen and C₁₋₆ alkyl. In some embodiments, R^{A} is C₁₋₆ alkyl optionally substituted with —N(R¹³)₂; and R¹³ is C₁₋₆ alkyl.

In some embodiments, R^{B} is selected from hydrogen and C₁₋₆ alkyl optionally substituted with one or more substituents independently selected from: halogen, —OR¹³, and —N(R¹³)₂; and R¹³ is selected from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R^{B} is C₁₋₆ alkyl optionally substituted with one or more substituents independently selected from: halogen, —OR¹³, and —N(R¹³)₂; and R¹³ is selected from hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R^{B} is selected from C₁₋₆ alkyl optionally substituted with halogen, —OR¹³, and —N(R¹³)₂; and R¹³ is selected from hydrogen and C₁₋₆ alkyl. In some embodiments, R^{B} is C₁₋₆ alkyl optionally substituted with —N(R¹³)₂; and R¹³ is C₁₋₆ alkyl.

In some embodiments, R^{A} and R^{B} are each independently selected from C₁₋₆ alkyl optionally substituted with one or more substituents independently selected from: halogen, —OR¹³, —N(R¹³)₂, —C(O)R¹³, —C(O)OR¹³, —NO₂, and —CN; and each R¹³ is independently selected from C₁₋₆ alkyl, C₁₋₆ haloalkyl, C₁₋₆ hydroxyalkyl, C₁₋₆ alkoxy, and C₁₋₆ haloalkoxy.

In some embodiments, R^{A} and R^{B} come together to form a 4- to 10-membered heterocycle optionally substituted with one or more substituents independently selected from:
halogen, —OR¹¹, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
C₁₋₆ alkyl optionally substituted with one or more substituents independently selected from: halogen, —OR⁴, —SR¹⁴, —N(R¹⁴)₂, —C(O)R¹⁴, —C(O)OR¹⁴, —NO₂, and —CN; and
C₃₋₁₀ carbocycle and 3- to 10-membered heterocycle, any of which is optionally substituted with one or more substituents independently selected from: halogen, —OR⁴, —SR¹⁴, —N(R¹⁴)₂, —C(O)R¹⁴, —C(O)OR¹⁴, —NO₂, —CN; and C₁₋₆ alkyl optionally substituted with one or more substituents independently selected from: halogen, —OR¹¹, —SR¹⁴, —N(R¹⁴)₂, —C(O)R¹⁴, —C(O)OR¹⁴, —NO₂, and —CN.

In some embodiments, R^{A} and R^{B} come together to form a 4- to 10-membered heterocycle optionally substituted with one or more substituents independently selected from:
halogen, —OR¹¹, —SR¹⁴, —N(R¹⁴)₂, —C(O)R¹⁴, —C(O)OR¹⁴, —NO₂, and —CN;
C₁₋₆ alkyl optionally substituted with one or more substituents independently selected from: halogen, —OR¹¹, —SR¹⁴, —N(R¹⁴)₂, —C(O)R¹⁴, —C(O)OR¹⁴, —NO₂, and —CN; and
C₃₋₆ carbocycle and 3- to 6-membered heterocycle, any of which is optionally substituted with one or more substituents independently selected from: halogen, —OR¹¹, —SR¹⁴, —N(R¹⁴)₂, —C(O)R¹⁴, —C(O)OR¹⁴, —NO₂, —CN, C₁₋₆ alkyl, and C₁₋₆ haloalkyl.

In some embodiments, R^{A} and R^{B} come together to form a 4- to 10-membered heterocycle optionally substituted with one or more substituents independently selected from:
halogen, —OR¹¹, —N(R¹⁴)₂, and —CN;
C₁₋₆ alkyl optionally substituted with one or more substituents independently selected from: halogen, —OR¹⁴, —N(R¹⁴)₂, and —CN; and 3- to 6-membered heterocycle optionally substituted with one or more substituents independently selected from: halogen, —OR¹⁴, —N(R¹⁴)₂, C₁₋₆ alkyl, and C₁₋₆ haloalkyl.

In some embodiments, R^{A} and R^{B} come together to form a 4- to 10-membered heterocycle optionally substituted with one or more substituents independently selected from: halogen, —OR¹⁴, —N(R¹⁴)₂, —CN, C₁₋₆ alkyl, C₁₋₆ haloalkyl, and 6-membered heterocycle optionally substituted with one or more substituents independently selected from: halogen, —OR¹⁴, —N(R¹⁴)₂, C₁₋₆ alkyl, and C₁₋₆ haloalkyl. In some embodiments, R^{A} and R^{B} come together to form a 4- to 10-membered heterocycle optionally substituted with one or more substituents independently selected from: halogen, —OR¹⁴, —N(R¹⁴)₂, —CN, C₁₋₆ alkyl, C₁₋₆ haloalkyl, and 6-membered heterocycle optionally substituted with one or more C₁₋₆ alkyl.

In some embodiments, R^{A} and R^{B} come together to form a 4- to 6-membered heterocycle optionally substituted with one or more substituents independently selected from:
halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN; and
C₃₋₁₀ carbocycle and 3- to 6-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN.

In some embodiments, R^{A} and R^{B} come together to form a 5- to 6-membered heterocycle optionally substituted with one or more substituents independently selected from:
halogen, —OR¹⁴, —SR¹⁴, —N(R¹⁴)₂, —C(O)R¹⁴, —C(O)OR¹⁴, —NO₂, and —CN;
C₁₋₆ alkyl optionally substituted with one or more substituents independently selected from: halogen, —OR¹⁴, —SR¹⁴ —N(R¹⁴)₂, —C(O)R¹⁴, —C(O)OR¹⁴, —NO₂, and —CN; and
C₃₋₆ carbocycle and 5- to 6-membered heterocycle, any of which is optionally substituted with one or more substituents independently selected from: halogen, —OR¹⁴, —SR¹⁴ —N(R¹⁴)₂, —C(O)R¹⁴, —C(O)OR¹⁴, —NO₂, —CN, C₁₋₆ alkyl, and C₁₋₆ haloalkyl.

In some embodiments, R^{A} and R^{B} come together to form a 5- to 6-membered heterocycle optionally substituted with one or more substituents independently selected from:
halogen, —OR¹⁴, —N(R¹⁴)₂, and —CN;
C₁₋₆ alkyl optionally substituted with one or more substituents independently selected from: halogen, —OR¹⁴, —N(R¹⁴)₂, and —CN; and
5- to 6-membered heterocycle optionally substituted with one or more substituents independently selected from: halogen, —OR¹⁴, —N(R¹⁴)₂, C₁₋₆ alkyl, and C₁₋₆ haloalkyl.

In some embodiments, R^{A} and R^{B} come together to form a 5- to 6-membered heterocycle optionally substituted with one or more substituents independently selected from: halogen, —OR¹⁴, —N(R¹⁴)₂, —CN, C₁₋₆ alkyl, C₁₋₆ haloalkyl, and 6-membered heterocycle optionally substituted with one or more substituents independently selected from: halogen, —OR¹⁴, —N(R¹⁴)₂, C₁₋₆ alkyl, and C₁₋₆ haloalkyl. In some embodiments, R^{A} and R^{B} come together to form a 6-membered heterocycle optionally substituted with one or more substituents independently selected from: halogen, —OR¹⁴, —N(R¹⁴)₂, —CN, C₁₋₆ alkyl, C₁₋₆ haloalkyl, and 6-membered heterocycle optionally substituted with one or more C₁₋₆ alkyl. In some embodiments, R^{A} and R^{B} come together to form a 6-membered heterocycle optionally substituted with one or more substituents independently selected from 6-membered heterocycle optionally substituted with one or more C₁₋₆ alkyl.

In some embodiments, R^{A} and R^{B} come together to form a 4- to 6-membered heterocycle optionally substituted with one or more substituents independently selected from: halogen, —OR¹⁴, —N(R¹⁴)₂, —C(O)R¹⁴, —C(O)OR¹⁴, —NO₂, and —CN; and C₁₋₆ alkyl optionally substituted with one or more substituents independently selected from: halogen, —OR¹⁴ —N(R¹⁴)₂, —C(O)R¹⁴, —C(O)OR¹⁴, —NO₂, and —CN.

In some embodiments, R^{A} and R^{B} come together to form a 4- to 6-membered heterocycle selected from: azetidine, pyrrolidine, piperidine, morpholine, and thiomorpholine, each of which is optionally substituted with one or more substituents independently selected from: halogen, —OR¹⁴, —N(R¹⁴)₂, —C(O)R¹⁴, —C(O)OR¹⁴, —NO₂, and —CN; and C₁₋₆ alkyl optionally substituted with one or more substituents independently selected from: halogen, —OR¹¹, —N(R¹⁴)₂, —C(O)R¹⁴, —C(O)OR¹⁴, —NO₂, and —CN. In some embodiments, R^{A} and R^{B} come together to form a 4- to 6-membered heterocycle selected from: azetidine, pyrrolidine, piperidine, morpholine, and thiomorpholine, each of which is optionally substituted with and C₁₋₆ alkyl optionally substituted with one or more substituents independently selected from: halogen, —OR¹⁴, —N(R¹⁴)₂, —C(O)R¹⁴, —C(O)OR¹⁴, —NO₂, and —CN.

In some embodiments, A is selected from a monocyclic 5- to 6-membered heteroaryl, bicyclic heteroaryl, bicyclic aryl, and phenyl, any of which are optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN. In some embodiments, A is absent.

In some embodiments, A is selected from monocyclic 5- to 6-membered heteroaryl and bicyclic heteroaryl, any of which is optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN.

In some embodiments, A is a monocyclic 5- to 6-membered heteroaryl selected from pyrrole, pyrazole, imidazole, triazole, tetrazole, oxazole, isoxazole, isothiazole, thiazole, oxadiazole, thiadiazole, pyridine, pyridazine, pyrimidine, pyrazine, and triazine, any of which is optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN.

In some embodiments, A is a monocyclic 5- to 6-membered heteroaryl selected from pyrazole optionally substituted with C₁₋₆ alkyl optionally substituted with one or more substituents independently selected from: halogen, —OR¹⁰, —N(R¹⁰)₂, —NO₂, and —CN; and R¹⁰ is selected from hydrogen, C₁₋₆ alkyl, C₁₋₆ haloalkyl, C₁₋₆ hydroxyalkyl, C₁₋₆ alkoxy, and C₁₋₆ haloalkoxy.

In some embodiments, A is selected from phenyl optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN.

In some embodiments, A is selected from bicyclic aryl optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN.

In some embodiments, A is a bicyclic heteroaryl selected from indole, isoindole, indolizine, indazole, benzimidazole, azaindole, purine, benzisoxazole, benzoxazole, benzisothiazole, benzoisothiazole, imidazopyridine, thiazolepyridine, and oxazolepyridine, any one of which is optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN.

In some embodiments, A is selected from indole, isoindole, indolizine benzimidazole any one of which is optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN. In some embodiments, the bicyclic heteroaryl of A is an optionally substituted indole. In some embodiments, A is selected from indole optionally substituted with one or more substituents selected from: halogen, —CN, —NO₂, —NH₂; and C₁₋₆ alkyl optionally substituted with one or more substituents independently selected from: halogen, —OR¹⁰, —N(R¹⁰)₂, —NO₂, and —CN.

In some embodiments, L is selected from —O—, N(R¹⁵), S(R¹⁶); and C₁₋₆ alkylene optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹ —S(O)₂R¹¹, —S(O)₂N(R¹¹)₂, —NO₂, and —CN. In some embodiments, L is C₁₋₆ alkylene optionally substituted with one or more substituents independently selected from: halogen, —OR¹¹—N(R¹¹)₂, —C(O)R¹¹, —NO₂, and —CN. In some embodiments, L is selected from —O—, N(R¹⁵), S(R¹⁶). In some embodiments, L is a bond.

In some aspects, the present disclosure provides compounds represented by the structure of Formula (IA):

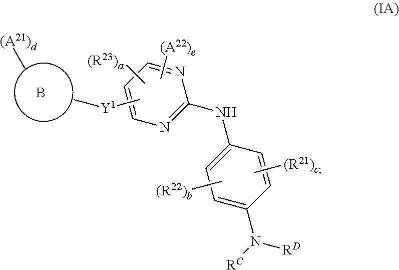

or a pharmaceutically acceptable salt thereof, wherein:
Y¹ is selected from a bond, —O—, —S—, and —N(R²⁴)—;
B is selected from a monocyclic 5- to 6-membered heteroaryl, bicyclic heteroaryl, bicyclic aryl, and phenyl, any of which is optionally substituted with one or more R²;
   A²¹ is represented by -L²¹-C²¹;
   A²² is represented by -L²²-C²²;
   L²¹ and L²² are each independently selected from a bond and -L^{A}-L^{B}-;
      L^{A} is selected from a bond, —O—, —S—, and —N(R²⁶)—; and
      L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹ —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), —S(O)R³¹, —S(O)₂R³¹, —S(O)₂N(R³¹)₂, ═O, ═S, ═NR³¹, —NO₂, and —CN;
   C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —S(O)R³², —S(O)₂R³², —S(O)₂N(R³²)₂, —NO₂, and —CN;
   wherein at least one of C²¹, C²² and B is substituted by —C(O)H;
d is selected from 0 and 1;
e is selected from 0 and 1;
R²¹ is an electrophile;
R²² is independently selected at each occurrence from:
   halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁴, —SR³⁴ —; N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, ═O, ═S, ═NR³⁴, —NO₂, and —CN;
R²³ is independently selected at each occurrence from:
   halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁵, —SR³⁵ —; N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, ═O, ═S, ═NR³⁵, —NO₂, and —CN;
R²⁴ is selected from hydrogen, C₁₋₄ alkyl, and C₁₋₄ haloalkyl;
R²⁵ is independently selected at each occurrence from:
   halogen, —OR³⁶, —SR³⁶, —N(R³⁶)₂, —C(O)H, —C(O)R³⁷, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁶, —SR³⁶ —; N(R³⁶)₂, —C(O)R³⁶, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, ═O, ═S, ═NR³⁶, —NO₂, and —CN;
R²⁶ is selected at each occurrence from hydrogen, C₁₋₄ alkyl, and C₁₄ haloalkyl;
a is selected from 0, 1, and 2.
b is selected from 0, 1, 2, 3, and 4;
c is selected from 0 and 1;
R^{C} and R^{D} are each independently selected from hydrogen and C₁₋₆ alkyl optionally substituted with one or more substituents independently selected from: halogen, —OR³⁸, —SR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), —S(O)R³⁸, —S(O)₂R³⁸, —S(O)₂N(R³⁸)₂, ═O, ═S, ═NR³⁸, —NO₂, and —CN; or
R^{C} and R^{D} come together to form a 4- to 10-membered heterocycle optionally substituted with one or more substituents independently selected from:
   halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
   C₁₋₆ alkyl optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹ —; C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy; and
R³³ and R³⁷ are each independently selected at each occurrence from: C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some aspects, the present disclosure provides compounds represented by the structure of Formula (II):

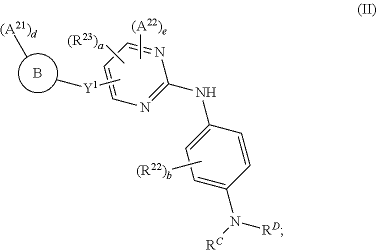

or a pharmaceutically acceptable salt thereof, wherein:
Y¹ is selected from a bond, —O—, —S—, and —N(R²⁴)—;
B is selected from a monocyclic 5- to 6-membered heteroaryl, bicyclic heteroaryl, bicyclic aryl, and phenyl, any of which is optionally substituted with one or more R²;
A²¹ is represented by -L²¹-C²¹;
A²² is represented by -L²²-C²²;
   L²¹ and L²² are each independently selected from a bond and -L^{A}-L^{B}-;
      L^{A} is selected from a bond, —O—, —S—, and —N(R²⁶)—; and
      L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹ —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), —S(O)R³¹, —S(O)₂R³¹, —S(O)₂N(R³¹)₂, ═O, ═S, ═NR³¹, —NO₂, and —CN;
   C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —S(O)R³², —S(O)₂R³², —S(O)₂N(R³²)₂, —NO₂, and —CN;
   wherein at least one of C²¹, C²² and B is substituted by —C(O)H;
d is selected from 0 and 1;
e is selected from 0 and 1;
R²² is independently selected at each occurrence from:
   halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁴, —SR³⁴ —; N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, ═O, ═S, ═NR³⁴, —NO₂, and —CN;
R²³ is independently selected at each occurrence from:
   halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁵, —SR³⁵ —; N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, ═O, ═S, ═NR³⁵, —NO₂, and —CN;
R²⁴ is selected from hydrogen, C₁₋₄ alkyl, and C₁₋₄ haloalkyl;
R²⁵ is independently selected at each occurrence from:
   halogen, —OR³⁶, —SR³⁶, —N(R³⁶)₂, —C(O)H, —C(O)R³⁷, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁶, —SR³⁶ —; N(R³⁶)₂, —C(O)R³⁶, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, ═O, ═S, ═NR³⁶, —NO₂, and —CN;
R²⁶ is selected at each occurrence from hydrogen, C₁₋₄ alkyl, and C₁₄ haloalkyl;
a is selected from 0, 1, and 2.
b is selected from 0, 1, 2, 3, and 4;
R^{C} and R^{D} are each independently selected from hydrogen and C₁₋₆ alkyl optionally substituted with one or more substituents independently selected from: halogen, —OR³⁸, —SR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), —S(O)R³⁸, —S(O)₂R³⁸, —S(O)₂N(R³⁸)₂, ═O, ═S, ═NR³⁸, —NO₂, and —CN; or
R^{C} and R^{D} come together to form a 4- to 10-membered heterocycle optionally substituted with one or more substituents independently selected from:
   halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
   C₁₋₆ alkyl optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹ —; C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy; and
R³³ and R³⁷ are each independently selected at each occurrence from: C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, the present disclosure provides compounds represented by the structure of Formula (II):

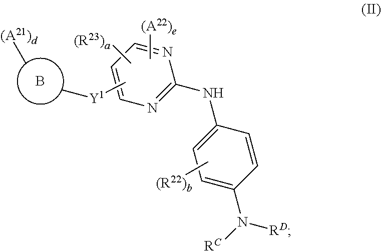

or a pharmaceutically acceptable salt thereof, wherein:
Y¹ is selected from a bond, —O—, and —N(R²⁴)—;
B is selected from a monocyclic 5- to 6-membered heteroaryl, bicyclic heteroaryl, bicyclic aryl, and phenyl, any of which is optionally substituted with one or more R²;
A²¹ is represented by -L²¹-C²¹;
A²² is represented by -L²²-C²²;
   L²¹ and L²² are each independently selected from a bond and -L^{A}-L^{B}-;
      L^{A} is selected from a bond and —N(R²⁶)—; and
      L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹ —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), ═O, ═NR³¹, —NO₂, and —CN;
   C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —NO₂, and —CN;
   wherein at least one of C²¹, C²², and B is substituted by —C(O)H;
d is selected from 0 and 1;
e is selected from 0 and 1;
R²² is independently selected at each occurrence from:
   halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —NO₂, and —CN; and
   C₁₋₆ alkyl, C₂₋₆ alkenyl, and C₂₋₆ alkynyl, each of which is optionally substituted with one or more substituents independently selected from: halogen, —OR³⁴, —SR³⁴ —; N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), ═O, ═NR³⁴, —NO₂, and —CN;
R²³ is independently selected at each occurrence from:
   halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —NO₂, and —CN; and
   C₁₋₆ alkyl, C₂₋₆ alkenyl, and C₂₋₆ alkynyl, each of which is optionally substituted with one or more substituents independently selected from: halogen, —OR³⁵, —SR³⁵ —; N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), ═O, ═NR³⁵, —NO₂, and —CN;
R²⁴ is selected from hydrogen, C₁₋₄ alkyl, and C₁₋₄ haloalkyl;
R²⁵ is independently selected at each occurrence from:
   halogen, —OR³⁶, —SR³⁶, —N(R³⁶)₂, —C(O)H, —C(O)R³⁷, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —P(O)(OR³⁶)₂, —P(O)R³⁶(OR³⁶), —P(O)(R³⁶)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁶, —SR³⁶ —; N(R³⁶)₂, —C(O)R³⁶, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —P(O)(OR³⁶)₂, —P(O)R³⁶(OR³⁶), —P(O)(R³⁶)₂, ═O, ═NR³⁶, —NO₂, and —CN;
R²⁶ is selected at each occurrence from hydrogen, C₁₋₄ alkyl, and C₁₄ haloalkyl;
a is selected from 0, 1, and 2.
b is selected from 0, 1, 2, 3, and 4;
R^{C} and R^{D} are each independently selected from hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), ═O, and ═NR³⁸; or
R^{C} and R^{D} come together to form a 4- to 10-membered heterocycle optionally substituted with one or more substituents independently selected from:
   halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN;
   C₁₋₆ alkyl optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, and ═NR³⁹; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹ —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, and ═NR³⁹;
R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy; and
R³³ and R³⁷ are each independently selected at each occurrence from: C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, the present disclosure provides compounds represented by the structure of Formula (II):

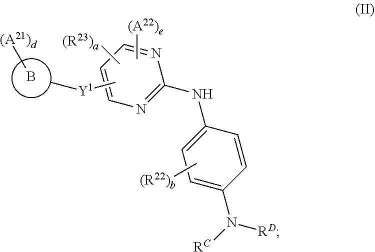

or a pharmaceutically acceptable salt thereof, wherein:
Y¹ is selected from a bond, —O—, and —N(R²⁴)—;
B is selected from a bicyclic heteroaryl and phenyl, any of which is optionally substituted with one or more R²⁵;
A²¹ is represented by -L²¹-C²¹;
A²² is represented by -L²²-C²²;
   L²¹ and L²² are each independently selected from a bond and -L^{A}-L^{B}-;
      L^{A} is selected from a bond and —N(R²⁶)—; and
      L^{B} is selected from C₁₋₄ alkylene and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), ═O, ═NR³¹, —NO₂, and —CN;
   C²¹ and C²² are each independently selected from phenyl optionally substituted with one or more substituents independently selected from: halogen, —OR³², —SR³² —; N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —NO₂, and —CN;
   wherein at least one of C²¹, C²², and B is substituted by —C(O)H;
d is selected from 0 and 1;
e is selected from 0 and 1;
R²² is independently selected at each occurrence from:
   halogen, —OR³⁴, —N(R³⁴)₂, —C(O)R³⁴, —C(O)OR³⁴, —OC(O)R³⁴, —C(O)N(R³⁴)₂, —N(R³⁴)C(O)R³⁴, —N(R³⁴)C(O)OR³⁴, —N(R³⁴)S(O)₂(R³⁴), —NO₂, and —CN; and
   C₁₋₆ alkyl, C₂₋₆ alkenyl, and C₂₋₆ alkynyl, each of which is optionally substituted with one or more substituents independently selected from: halogen, —OR³⁴, —N(R³⁴)₂, —C(O)R³⁴, —C(O)OR³⁴, —OC(O)R³⁴, —C(O)N(R³⁴)₂, —N(R³⁴)C(O)R³⁴, —N(R³⁴)S(O)₂(R³⁴), and ═O;
R²³ is independently selected at each occurrence from:
   halogen, —OR³⁵, —N(R³⁵)₂, —C(O)R³⁵, —C(O)OR³⁵, —OC(O)R³⁵, —C(O)N(R³⁵)₂, —N(R³⁵)C(O)R³⁵, —N(R³⁵)S(O)₂(R³⁵), —NO₂, and —CN; and
   C₁₋₆ alkyl, C₂₋₆ alkenyl, and C₂₋₆ alkynyl, each of which is optionally substituted with one or more substituents independently selected from: halogen, —OR³⁵, —N(R³⁵)₂, —C(O)R³⁵, —C(O)OR³⁵, —OC(O)R³⁵, —C(O)N(R³⁵)₂, —N(R³⁵)C(O)R³⁵, —N(R³⁵)S(O)₂(R³⁵), and ═O;
R²⁴ is selected from hydrogen, C₁₋₄ alkyl, and C₁₋₄ haloalkyl;
R²⁵ is independently selected at each occurrence from:
   halogen, —OR³⁶, —N(R³⁶)₂, —C(O)H, —C(O)R³⁷, —C(O)OR³⁶, —OC(O)R³⁶, —C(O)N(R³⁶)₂, —N(R³⁶)C(O)R³⁶, —N(R³⁶)S(O)₂(R³⁶), —P(O)(OR³⁶)₂, —P(O)R³⁶(OR³⁶), —P(O)(R³⁶)₂, —NO₂, and —CN; and
   C₁₋₆ alkyl, C₂₋₆ alkenyl, and C₂₋₆ alkynyl, each of which is optionally substituted with one or more substituents independently selected from: halogen, —OR³⁶, —N(R³⁶)₂. —C(O)R³⁶, —C(O)OR³⁶, —OC(O)R³⁶, —C(O)N(R³⁶)₂, —N(R³⁶)C(O)R³⁶, —N(R³⁶)S(O)₂(R³⁶), —P(O)(OR³⁶)₂, —P(O)R³⁶(OR³⁶), —P(O)(R³⁶)₂, and ═O;
R²⁶ is selected at each occurrence from hydrogen, C₁₋₄ alkyl, and C₁₄ haloalkyl;
a is selected from 0, 1, and 2.
b is selected from 0, 1, 2, 3, and 4;
R^{C} and R^{D} are each independently selected from hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —C(O)N(R³⁸)₂, —N(R³⁸)C(O)R³⁸, —N(R³⁸)S(O)₂(R³⁸), and ═O; or
R^{C} and R^{D} come together to form a 4- to 10-membered heterocycle optionally substituted with one or more substituents independently selected from:
   halogen, —OR³⁹, —N(R³⁹)₂, —C(O)R³⁹, —C(O)OR³⁹, —OC(O)R³⁹, —C(O)N(R³⁹)₂, —N(R³⁹)C(O)R³⁹, —N(R³⁹)S(O)₂(R³⁹), ═O, ═NR³⁹, —NO₂, and —CN;
   C₁₋₆ alkyl optionally substituted with one or more substituents independently selected from: halogen, —OR³⁹, —N(R³⁹)₂, —C(O)R³⁹, —C(O)OR³⁹, —OC(O)R³⁹ —C(O)N(R³⁹)₂, —N(R³⁹)C(O)R³⁹, —N(R³⁹)S(O)₂(R³⁹), and ═O; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —C(O)OR³⁹, —OC(O)R³⁹, —C(O)N(R³⁹)₂, —N(R³⁹)C(O)R³⁹, —N(R³⁹)S(O)₂(R³⁹), ═O, and ═NR³⁹;
R³¹, R³², R³⁴, R³⁵, R³⁶, R³⁸, and R³⁹ are each independently selected at each occurrence from: hydrogen, C₁₋₆ alkyl, C₁₋₆ alkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, the present disclosure provides compounds represented by the structure of Formula (II):

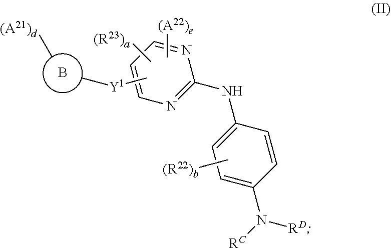

or a pharmaceutically acceptable salt thereof, wherein:
Y¹ is selected from a bond, —O—, and —N(R²⁴)—;
B is selected from a bicyclic heteroaryl and phenyl;
A²¹ is represented by -L²¹-C²¹;
A²² is represented by -L²²-C²²;
   L²¹ and L²² are each independently selected from a bond and -L^{A}-L^{B}-;
      L^{A} is selected from a bond and —N(R²⁶)—; and
      L^{B} is selected from C₁₋₄ alkylene and C₂₋₄ alkynylene;
   C²¹ and C²² are each independently selected from phenyl optionally substituted with one or more substituents independently selected from: halogen, —OR³², and —C(O)H;
   wherein at least one of C²¹, C²², and B is substituted by —C(O)H;
d is selected from 0 and 1;
e is selected from 0 and 1;
R²² is independently selected at each occurrence from —OR³⁴ and —N(R³⁴)C(O)R³⁴;
R²³ is independently selected at each occurrence from halogen;
R²⁴ is selected from hydrogen, C₁₋₄ alkyl, and C₁₋₄ haloalkyl;
R²⁵ is independently selected at each occurrence from:
   —OR³⁶, —C(O)H, and —P(O)(R³⁶)₂; and
   C₁₋₆ alkyl optionally substituted with one or more substituents independently selected from: halogen, —OR³⁶, —N(R³⁶)₂, —C(O)R³⁶, —C(O)OR³⁶, —OC(O)R³⁶ —; C(O)N(R³⁶)₂, —N(R³⁶)C(O)R³⁶, —N(R³⁶)S(O)₂(R³⁶), —P(O)(OR³⁶)₂, —P(O)R³⁶(OR³⁶), —P(O)(R³⁶)₂, and ═O;
R²⁶ is selected at each occurrence from hydrogen;
a is selected from 0 and 1.
b is selected from 1 and 2;
R^{C} and R^{D} are each independently selected from C₁₋₆ alkyl optionally substituted with one or more substituents independently selected from: —N(R³⁸)₂; or
R^{C} and R^{D} come together to form a 4- to 10-membered heterocycle optionally substituted with one or more substituents independently selected from:
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —C(O)OR³⁹, —OC(O)R³⁹, —C(O)N(R³⁹)₂, —N(R³⁹)C(O)R³⁹, —N(R³⁹)S(O)₂(R³⁹), ═O, and ═NR³⁹; and
R³¹, R³², R³⁴, R³⁵, R³⁶, R³⁸, and R³⁹ are each independently selected at each occurrence from: hydrogen and C₁₋₆ alkyl.

In some embodiments, the present disclosure provides compounds represented by the structure of Formula (II):

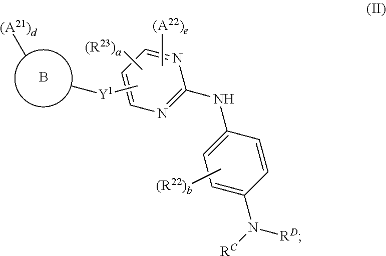

or a pharmaceutically acceptable salt thereof, wherein:
Y¹ is selected from a bond, —O—, and —N(R²⁴)—;
B is selected from a bicyclic heteroaryl and phenyl;
A²¹ is represented by -L²¹-C²¹
A²² is represented by -L²²-C²²;
   L²¹ and L²² are each independently selected from a bond and -L^{A}-L^{B}-;
      L^{A} is selected from a bond and —N(R²⁶)—; and
      L^{B} is selected from C₁₋₄ alkylene and C₂₋₄ alkynylene;
   C²¹ and C²² are each independently selected from phenyl optionally substituted with one or more substituents independently selected from: halogen, —OR³², and —C(O)H;
   wherein at least one of C²¹, C²², and B is substituted by —C(O)H;
d is selected from 0 and 1;
e is selected from 0 and 1;
R²² is independently selected at each occurrence from —OR³⁴ and —N(R³⁴)C(O)R³⁴;
R²³ is independently selected at each occurrence from halogen;
R²⁴ is selected from hydrogen, C₁₋₄ alkyl, and C₁₋₄ haloalkyl;
R²⁵ is independently selected at each occurrence from:
   —OR³⁶, —C(O)H, and —P(O)(R³⁶)₂; and
   C₁₋₆ alkyl optionally substituted with one or more substituents independently selected from: halogen, —OR³⁶, —N(R³⁶)₂, —C(O)R³⁶, —C(O)OR³⁶, —OC(O)R³⁶ —; C(O)N(R³⁶)₂, —N(R³⁶)C(O)R³⁶, —N(R³⁶)S(O)₂(R³⁶), —P(O)(OR³⁶)₂, —P(O)R³⁶(OR³⁶), —P(O)(R³⁶)₂, and ═O;
R²⁶ is selected at each occurrence from hydrogen;
a is selected from 0 and 1.
b is selected from 1 and 2;
R^{C} and R^{D} are each independently selected from C₁₋₆ alkyl optionally substituted with one or more substituents independently selected from: —N(R³⁸)₂; or
R^{C} and R^{D} come together to form a 4- to 10-membered heterocycle optionally substituted with one or more substituents independently selected from:
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —C(O)OR³⁹, —OC(O)R³⁹, —C(O)N(R³⁹)₂, —N(R³⁹)C(O)R³⁹, —N(R³⁹)S(O)₂(R³⁹), ═O, and ═NR³⁹; and
R³¹, R³², R³⁴, R³⁵, R³⁶, R³⁸, and R³⁹ are each independently selected at each occurrence from: hydrogen and C₁₋₆ alkyl.

In some embodiments, c is 1. In some embodiments, c is 0. In some embodiments, d is 0. In some embodiments, d is 1. In some embodiments, e is 0. In some embodiments, e is 1. In some embodiments, Y¹ is selected from a bond, —O—, and —N(R⁴)—. In some embodiments, Y¹ is selected from a bond and —O—. In some embodiments, Y¹ is selected from a bond and —N(R⁴)—. In some embodiments, Y¹ is selected from —O—, —S—, and —N(R⁴)—. In some embodiments, Y¹ is selected from —O— and —N(R⁴)—. In some embodiments, Y¹ is —O—. In some embodiments, Y¹ is —N(R⁴)—. In some embodiments, Y¹ is a bond.

In some embodiments, B is selected from a monocyclic 5- to 6-membered heteroaryl, bicyclic heteroaryl, bicyclic aryl, and phenyl, any of which is optionally substituted with one or more R²⁵. In some embodiments, B is selected from a monocyclic 5- to 6-membered heteroaryl and bicyclic heteroaryl, any of which is optionally substituted with one or more R²⁵. In some embodiments, B is selected from bicyclic aryl and phenyl, any of which is optionally substituted with one or more R²⁵. In some embodiments, B is selected from a monocyclic 5- to 6-membered heteroaryl and phenyl, any of which is optionally substituted with one or more R²⁵ In some embodiments, B is selected from a bicyclic heteroaryl and bicyclic aryl, any of which is optionally substituted with one or more R²⁵. In some embodiments, B is selected from bicyclic heteroaryl and phenyl, any of which is optionally substituted with one or more R²⁵. In some embodiments, B is a monocyclic 5- to 6-membered heteroaryl optionally substituted with one or more R²⁵. In some embodiments, B is bicyclic heteroaryl optionally substituted with one or more R²⁵. In some embodiments, B is bicyclic aryl optionally substituted with one or more R²⁵. In some embodiments, B is phenyl optionally substituted with one or more R²⁵. In some embodiments, B is selected from indole and phenyl, any of which is optionally substituted with one or more R²⁵. In some embodiments, B is indole optionally substituted with one or more R²⁵

In some embodiments, R²⁵ is independently selected at each occurrence from:
halogen, —OR³⁶, —SR³⁶, —N(R³⁶)₂, —C(O)H, —C(O)R³⁷, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁶, —SR³⁶, —N(R³⁶)₂, —C(O)R³⁶, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, ═O, ═S, ═NR³⁶, —NO₂, and —CN.

In some embodiments, R²⁵ is independently selected at each occurrence from:
halogen, —OR³⁶, —SR³⁶, —N(R³⁶)₂, —C(O)H, —C(O)R³⁷, —C(O)OR³⁶, —OC(O)R³⁶, —C(O)N(R³⁶)₂, —N(R³⁶)C(O)R³⁶, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁶, —SR³⁶, —N(R³⁶)₂, —C(O)R³⁶, —C(O)OR³⁶, —OC(O)R³⁶, —C(O)N(R³⁶)₂, —N(R³⁶)C(O)R³⁶, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, ═O, ═S, ═NR³⁶, —NO₂, and —CN.

In some embodiments, R²⁵ is independently selected at each occurrence from:
halogen, —OR³⁶, —SR³⁶, —N(R³⁶)₂, —C(O)H, —C(O)R³⁷, —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, and —CN; and
C₁₋₆ alkyl optionally substituted with one or more substituents independently selected from: halogen, —OR³⁶, —SR³⁶, —N(R³⁶)₂, —C(O)R³⁶, —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, ═O, and —CN.

In some embodiments, R²⁵ is independently selected at each occurrence from: halogen, —OR³⁶, —C(O)H, —C(O)R³⁷, —P(O)(R³⁶)₂, and C₁₋₆ alkyl optionally substituted with one or more substituents independently selected from: halogen, —OR³⁶, —C(O)R³⁶, and —P(O)(R³⁶)₂. In some embodiments, R²⁵ is independently selected at each occurrence from: —OR³⁶, —C(O)H, —P(O)(R³⁶)₂, and C₁₋₆ alkyl optionally substituted with one or more substituents independently selected from: halogen, —OR³⁶, —C(O)R³⁶, and —P(O)(R³⁶)₂. In some embodiments, R²⁵ is independently selected at each occurrence from: —OR³⁶, —C(O)H, —P(O)(R³⁶)₂, and C₁₋₆ alkyl. In some embodiments, R²⁵ is independently selected at each occurrence from: methyl, —OH, —C(O)H, and —P(O)(CH₃)₂. In some embodiments, R²⁵ is independently selected at each occurrence from: —OR³⁶, —C(O)H, and —P(O)(R³⁶)₂. In some embodiments, R²⁵ is independently selected at each occurrence from: —OH, —C(O)H, and —P(O)(CH₃)₂. In some embodiments, R²⁵ is independently selected at each occurrence from C₁₋₆ alkyl optionally substituted with one or more substituents independently selected from: halogen, —OR³⁶, —C(O)R³⁶, and —P(O)(R³⁶)₂. In some embodiments, R²⁵ is independently selected at each occurrence from C₁₋₆ alkyl. In some embodiments, R²⁵ is independently selected at each occurrence from methyl.

In some embodiments, L²¹ and L²² are each independently selected from a bond and -L^{A}-L^{B}-, wherein:
L^{A} is selected from a bond, —O—, —S—, and —N(R²⁶)—; and
L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), —S(O)R³¹, —S(O)₂R³¹, —S(O)₂N(R³¹)₂, ═O, ═S, ═NR³¹, —NO₂, and —CN.

In some embodiments, L²¹ and L²² are each independently selected from a bond and -L^{A}-L^{B}-, wherein:
L^{A} is selected from a bond, —O—, —S—, and —N(R²⁶)—; and
L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), ═O, ═NR³¹, —NO₂, and —CN.

In some embodiments, L²¹ and L²² are each independently selected from a bond and -L^{A}-L^{B}-, wherein:
L^{A} is selected from a bond, —O—, —S—, and —N(R²⁶)—; and
L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹, —C(O)N(R³¹)₂, —N(R³¹)C(O)R³¹, —N(R³¹)S(O)₂(R³¹), ═O, ═NR³¹, —NO₂, and —CN.

In some embodiments, L²¹ and L²² are each independently selected from a bond and -L^{A}-L^{B}-, wherein:
L^{A} is selected from a bond and —N(R²⁶)—; and
L^{B} is selected from C₁₋₄ alkylene and C₂₋₄ alkynylene, any of which is optionally substituted with one or more substituents independently selected from: halogen, —OR³¹, —SR³¹ —; N(R³¹)₂, —C(O)R³¹, —C(O)OR³¹, —OC(O)R³¹, —C(O)N(R³¹)₂, —N(R³¹)C(O)R³¹, —N(R³¹)S(O)₂(R³¹), ═O, ═NR³¹, —NO₂, and —CN.

In some embodiments, L²¹ and L²² are each independently selected from a bond and -L^{A}-L^{B}-, wherein:
L^{A} is selected from a bond and —N(R²⁶)—; and
L^{B} is selected from C₁₋₄ alkylene and C₂₋₄ alkynylene.

In some embodiments, L^{A} is selected from a bond, —O—, —S—, and —N(R²⁶)—. In some embodiments, L^{A} is selected from a bond and —N(R²⁶)—. In some embodiments, L^{A} is a bond. In some embodiments, L^{A} is —N(R²⁶)—.

In some embodiments, L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹ —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), —S(O)R³¹, —S(O)₂R³¹, —S(O)₂N(R³¹)₂, ═O, ═S, ═NR³¹, —NO₂, and —CN. In some embodiments, L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), ═O, ═NR³¹, —NO₂, and —CN. In some embodiments, L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹ —C(O)OR³¹, —OC(O)R³¹, —C(O)N(R³¹)₂, —N(R³¹)C(O)R³¹, —N(R³¹)S(O)₂(R³¹),═O, ═NR³¹, —NO₂, and —CN. In some embodiments, L^{B} is selected from C₁₋₄ alkylene and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹, —C(O)N(R³¹)₂, —N(R³¹)C(O)R³¹, —N(R³¹)S(O)₂(R³¹), ═O, ═NR³¹, —NO₂, and —CN. In some embodiments, L^{B} is selected from C₁₋₄ alkylene and C₂₋₄ alkynylene.

In some embodiments, C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —S(O)R³², —S(O)₂R³², —S(O)₂N(R³²)₂, —NO₂, and —CN. In some embodiments, C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —NO₂, and —CN. In some embodiments, C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —N(R³²)C(O)R³², —N(R³²)C(O)OR³², —N(R³²)C(O)N(R³²)₂, —N(R³²)S(O)₂(R³²), —NO₂, and —CN. In some embodiments, C²¹ and C²² are each independently selected from phenyl optionally substituted with one or more substituents independently selected from: halogen, —OR³², —SR³², —N(R³²)₂, —C(O)H, —N(R³²)C(O)R³², —N(R³²)C(O)OR³², —N(R³²)C(O)N(R³²)₂, —N(R³²)S(O)₂(R³²), —NO₂, and —CN. In some embodiments, C²¹ and C²² are each independently selected from phenyl optionally substituted with one or more substituents independently selected from: halogen, —OR³², and —C(O)H. In some embodiments, C²¹ and C²² are each independently selected from phenyl substituted with one or more substituents independently selected from: fluoro, —OH, —OCH₃, and —C(O)H. In some embodiments, C²¹ and C²² are each independently selected from phenyl substituted with two or more substituents independently selected from: fluoro, —OH, —OCH₃, and —C(O)H. In some embodiments, C²¹ and C²² are each independently selected from:

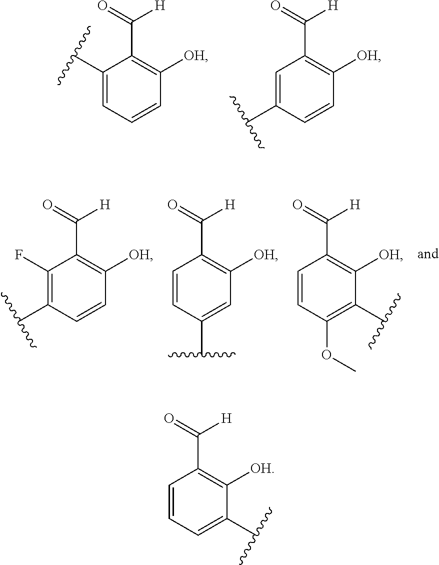

In some embodiments, b is selected from 0, 1, 2, 3, and 4. In some embodiments, b is selected from 0, 1, and 2. In some embodiments, b is selected from 1 and 2. In some embodiments, b is 0. In some embodiments, b is 1. In some embodiments, b is 2. In some embodiments, b is 3. In some embodiments, b is 4.

In some embodiments, R²² is independently selected at each occurrence from:
halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, ═O, ═S, ═NR³⁴, —NO₂, and —CN.

In some embodiments, R²² is independently selected at each occurrence from:
halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —NO₂, and —CN; and
C₁₋₆ alkyl, C₂₋₆ alkenyl, and C₂₋₆ alkynyl, each of which is optionally substituted with one or more substituents independently selected from: halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), ═O, ═NR³⁴, —NO₂, and —CN.

In some embodiments, R²² is independently selected at each occurrence from:
halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —NO₂, and —CN; and
C₁₋₆ alkyl, C₂₋₆ alkenyl, and C₂₋₆ alkynyl, each of which is optionally substituted with one or more substituents independently selected from: halogen, —OR³⁴, —SR³⁴, —N(R³⁴)₂, —C(O)R³⁴, —C(O)OR³⁴, —OC(O)R³⁴, —C(O)N(R³⁴)₂, —N(R³⁴)C(O)R³⁴, —N(R³⁴)S(O)₂(R³⁴), ═O, ═NR³⁴, —NO₂, and —CN.

In some embodiments, R²² is independently selected at each occurrence from:
halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —NO₂, and —CN; and
C₁₋₆ alkyl, C₂₋₆ alkenyl, and C₂₋₆ alkynyl, each of which is optionally substituted with one or more substituents independently selected from halogen.

In some embodiments, R²² is independently selected at each occurrence from: halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —NO₂, —CN, C₁₋₆ alkyl, and C₁₋₆ haloalkyl.

In some embodiments, R²² is independently selected at each occurrence from: halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, —NO₂, and —CN. In some embodiments, R²² is independently selected at each occurrence from: halogen, —OR³⁴, —SR³⁴, —N(R³⁴)₂, —C(O)R³⁴, —C(O)OR³⁴, —OC(O)R³⁴ —; OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —NO₂, and —CN. In some embodiments, R²² is independently selected at each occurrence from: —OR³⁴ and —N(R³⁴)C(O)R³⁴. In some embodiments, R²² is independently selected at each occurrence from: —OCH₃ and —N(H)C(O)CH₃.

In some embodiments, a is selected from 0, 1, and 2. In some embodiments, a is selected from 0 and 1. In some embodiments, a is 0. In some embodiments, a is 1. In some embodiments, a is 2.

In some embodiments, R²³ is independently selected at each occurrence from:
halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, ═O, ═S, ═NR³⁵, —NO₂, and —CN.

In some embodiments, R²³ is independently selected at each occurrence from:
halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), ═O, ═NR³⁵, —NO₂, and —CN.

In some embodiments, R²³ is independently selected at each occurrence from:
halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁵, —SR³⁵, —N(R³⁵)₂, —C(O)R³⁵, —C(O)OR³⁵, —OC(O)R³⁵, —C(O)N(R³⁵)₂, —N(R³⁵)C(O)R³⁵, —N(R³⁵)S(O)₂(R³⁵), ═O, ═NR³⁵, —NO₂, and —CN.

In some embodiments, R²³ is independently selected at each occurrence from:
halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
C₁₋₆ alkyl optionally substituted with one or more substituents independently selected from: halogen, —OR³⁵, —SR³⁵, —N(R³⁵)₂, —C(O)R³⁵, —C(O)OR³⁵, —OC(O)R³⁵, —C(O)N(R³⁵)₂, —N(R³⁵)C(O)R³⁵, —N(R³⁵)S(O)₂(R³⁵), ═O, ═NR³⁵, —NO₂, and —CN.

In some embodiments, R²³ is independently selected at each occurrence from: halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵I —S(O)₂R³⁵, —S(O)₂N(R³⁵)₂, —NO₂, —CN, C₁₋₆ alkyl and C₁₋₆ haloalkyl. In some embodiments, R²³ is independently selected at each occurrence from: halogen, —OR³⁵, —SR³⁵, —N(R³⁵)₂, —C(O)R³⁵ —; C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —NO₂, —CN, C₁₋₆ alkyl and C₁₋₆ haloalkyl. In some embodiments, R²³ is independently selected at each occurrence from: halogen, —OR³⁵, —SR³⁵ —; N(R³⁵)₂, —C(O)R³⁵, —C(O)OR³⁵, —OC(O)R³⁵, —C(O)N(R³⁵)₂, —N(R³⁵)C(O)R³⁵, —N(R³⁵)S(O)₂(R³⁵), —NO₂, —CN, C₁₋₆ alkyl and C₁₋₆ haloalkyl. In some embodiments, R²³ is independently selected at each occurrence from halogen. In some embodiments, each R²³ is chloro.

In some embodiments, R²⁴ is selected from hydrogen, C₁₋₄ alkyl, and C₁₄ haloalkyl. In some embodiments, R²⁴ is selected from hydrogen and C₁₋₄ alkyl. In some embodiments, R²⁴ is selected from hydrogen and C₁₋₄ haloalkyl. In some embodiments, R²⁴ is selected from C₁₄ alkyl and C₁₋₄ haloalkyl. In some embodiments, R²⁴ is hydrogen. In some embodiments, R²⁴ is C₁₋₄ alkyl. In some embodiments, R²⁴ is C₁₄ haloalkyl.

In some embodiments, R²⁶ is selected from hydrogen, C₁₋₄ alkyl, and C₁₄ haloalkyl. In some embodiments, R²⁶ is selected from hydrogen and C₁₋₄ alkyl. In some embodiments, R²⁶ is selected from hydrogen and C₁₋₄ haloalkyl. In some embodiments, R²⁶ is selected from C₁₄ alkyl and C₁₋₄ haloalkyl. In some embodiments, R²⁶ is hydrogen. In some embodiments, R²⁶ is C₁₋₄ alkyl. In some embodiments, R²⁶ is C₁₄ haloalkyl.

In some embodiments, R^{C} and R^{D} are each independently selected from: hydrogen and C₁₋₆ alkyl optionally substituted with one or more substituents independently selected from:
halogen, —OR³⁸, —SR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), —S(O)R³⁸, —S(O)₂R³⁸, —S(O)₂N(R³⁸)₂, ═O, ═S, ═NR³⁸, —NO₂, and —CN; or
R^{C} and R^{D} come together to form a 4- to 10-membered heterocycle optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
   C₁₋₆ alkyl optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹ —; C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN.

In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), ═O, ═NR³⁸, —NO₂, and —CN; or
R^{C} and R^{D} come together to form a 4- to 10-membered heterocycle optionally substituted with one or more substituents independently selected from: halogen, —OR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN;
   C₁₋₆ alkyl optionally substituted with one or more substituents independently selected from: halogen, —OR³⁹, —N(R³⁹)₂, —C(O)R³⁹, —C(O)OR³⁹, —OC(O)R³⁹ —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN.

In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —C(O)N(R³⁸)₂, —N(R³⁸)C(O)R³⁸, —N(R³⁸)S(O)₂(R³⁸), ═O, ═NR³⁸, —NO₂, and —CN; or
R^{C} and R^{D} come together to form a 4- to 10-membered heterocycle optionally substituted with one or more substituents independently selected from: halogen, —OR³⁹, —N(R³⁹)₂, —C(O)R³⁹, —C(O)OR³⁹, —OC(O)R³⁹, —C(O)N(R³⁹)₂, —N(R³⁹)C(O)R³⁹, —N(R³⁹)S(O)₂(R³⁹), ═O, ═NR³⁹, and —CN;
   C₁₋₆ alkyl optionally substituted with one or more substituents independently selected from: halogen, —OR³⁹, —N(R³⁹)₂, —C(O)R³⁹, —C(O)OR³⁹, —OC(O)R³⁹ —C(O)N(R³⁹)₂, —N(R³⁹)C(O)R³⁹, —N(R³⁹)S(O)₂(R³⁹), ═O, and ═NR³⁹; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —C(O)OR³⁹, —OC(O)R³⁹, —C(O)N(R³⁹)₂, —N(R³⁹)C(O)R³⁹, —N(R³⁹)S(O)₂(R³⁹), ═O, ═NR³⁹, and —CN.

In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)N(R³⁸)₂, —N(R³⁸)C(O)R³⁸, ═O, and —CN; or
R^{C} and R^{D} come together to form a 4- to 10-membered heterocycle optionally substituted with one or more substituents independently selected from:
   halogen, —OR³⁹, —N(R³⁹)₂, —C(O)R³⁹, —C(O)N(R³⁹)₂, —N(R³⁹)C(O)R³⁹, ═O, —CN, C₁₋₆ alkyl, and C₁₋₆ haloalkyl;
   C₃₋₁₀ carbocycle and 3- to 10-membered heterocycle, any of which is optionally substituted with one or more substituents independently selected from: halogen, C₁₋₆ alkyl, C₁₋₆ haloalkyl, —OR³⁹, —N(R³⁹)₂, —C(O)R³⁹, —C(O)N(R³⁹)₂, —N(R³⁹)C(O)R³⁹, ═O, and —CN.

In some embodiments, R^{C} and R^{D} are each independently selected from hydrogen; and C₁₋₆ alkyl optionally substituted with one or more substituents independently selected from: halogen, —OR³⁸, —N(R³⁸)₂, —C(O)R³⁸, —C(O)N(R³⁸)₂, —N(R³⁸)C(O)R³⁸, ═O, and —CN; or
R^{C} and R^{D} come together to form a 4- to 10-membered heterocycle optionally substituted with one or more substituents independently selected from:
   halogen, C₁₋₆ alkyl, and C₁₋₆ haloalkyl, —OR³⁹, —N(R³⁹)₂, —C(O)R³⁹, —C(O)N(R³⁹)₂, —N(R³⁹)C(O)R³⁹, ═O, and —CN; and
   C₃₋₁₀ carbocycle and 3- to 10-membered heterocycle, any of which is optionally substituted with one or more substituents independently selected from: halogen, C₁₋₆ alkyl, C₁₋₆ haloalkyl, —OR³⁹, —N(R³⁹)₂, —C(O)R³⁹, —C(O)N(R³⁹)₂, —N(R³⁹)C(O)R³⁹, ═O, and —CN.

In some embodiments, R^{C} and R^{D} are each independently selected from hydrogen; and C₁₋₆ alkyl optionally substituted with one or more substituents independently selected from —N(R³⁸)₂; or
R^{C} and R^{D} come together to form a 4- to 10-membered heterocycle optionally substituted with one or more substituents independently selected from 3- to 10-membered heterocycle optionally substituted with one or more substituents independently selected from C₁₋₆ alkyl.

In some embodiments, R^{C} and R^{D} are each independently selected from: hydrogen and C₁₋₆ alkyl optionally substituted with one or more substituents independently selected from: halogen, —OR³⁸, —SR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), —S(O)R³⁸, —S(O)₂R³⁸, —S(O)₂N(R³⁸)₂, ═O, ═S, ═NR³⁸, —NO₂, and —CN. In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), ═O, ═NR³⁸, —NO₂, and —CN. In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —C(O)N(R³⁸)₂, —N(R³⁸)C(O)R³⁸, —N(R³⁸)S(O)₂(R³⁸), ═O, ═NR³⁸, —NO₂, and —CN. In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)N(R³⁸)₂, —N(R³⁸)C(O)R³⁸, ═O, and —CN. In some embodiments, R^{C} and R^{D} are each independently selected from hydrogen; and C₁₋₆ alkyl optionally substituted with one or more substituents independently selected from —N(R³⁸)₂.

In some embodiments, R^{C} and R^{D} come together to form a 4- to 10-membered heterocycle optionally substituted with one or more substituents independently selected from:
halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
C₁₋₆ alkyl optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN; and
C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹ —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN.

In some embodiments, R^{C} and R^{D} come together to form a 4- to 10-membered heterocycle optionally substituted with one or more substituents independently selected from:
halogen, —OR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN;
C₁₋₆ alkyl optionally substituted with one or more substituents independently selected from: halogen, —OR³⁹, —N(R³⁹)₂, —C(O)R³⁹, —C(O)OR³⁹, —OC(O)R³⁹ —; OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN; and
C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —; C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN.

In some embodiments, R^{C} and R^{D} come together to form a 4- to 10-membered heterocycle optionally substituted with one or more substituents independently selected from:
halogen, —OR³⁹, —N(R³⁹)₂, —C(O)R³⁹, —C(O)OR³⁹, —OC(O)R³⁹, —C(O)N(R³⁹)₂, —N(R³⁹)C(O)R³⁹, —N(R³⁹)S(O)₂(R³⁹), ═O, ═NR³⁹, and —CN;
C₁₋₆ alkyl optionally substituted with one or more substituents independently selected from: halogen, —OR³⁹, —N(R³⁹)₂, —C(O)R³⁹, —C(O)OR³⁹, —OC(O)R³⁹ —; C(O)N(R³⁹)₂, —N(R³⁹)C(O)R³⁹, —N(R³⁹)S(O)₂(R³⁹), ═O, and ═NR³⁹; and
C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —C(O)OR³⁹, —OC(O)R³⁹, —C(O)N(R³⁹)₂, —N(R³⁹)C(O)R³⁹, —N(R³⁹)S(O)₂(R³⁹), ═O, ═NR³⁹, and —CN.

In some embodiments, R^{C} and R^{D} come together to form a 4- to 10-membered heterocycle optionally substituted with one or more substituents independently selected from: halogen, —OR³⁹, —N(R³⁹)₂, —C(O)R³⁹, —C(O)N(R³⁹)₂, —N(R³⁹)C(O)R³⁹, ═O, —CN, C₁₋₆ alkyl, and C₁₋₆ haloalkyl; and
C₃₋₁₀ carbocycle and 3- to 10-membered heterocycle, any of which is optionally substituted with one or more substituents independently selected from: halogen, C₁₋₆ alkyl, C₁₋₆ haloalkyl, —OR³⁹, —N(R³⁹)₂, —C(O)R³⁹, —C(O)N(R³⁹)₂, —N(R³⁹)C(O)R³⁹, ═O, and —CN.

In some embodiments, R^{C} and R^{D} come together to form a 4- to 10-membered heterocycle optionally substituted with one or more substituents independently selected from: halogen, C₁₋₆ alkyl, and C₁₋₆ haloalkyl, —OR³⁹, —N(R³⁹)₂, —C(O)R³⁹, —C(O)N(R³⁹)₂, —N(R³⁹)C(O)R³⁹, ═O, and —CN; and
C₃₋₁₀ carbocycle and 3- to 10-membered heterocycle, any of which is optionally substituted with one or more substituents independently selected from: halogen, C₁₋₆ alkyl, C₁₋₆ haloalkyl, —OR³⁹, —N(R³⁹)₂, —C(O)R³⁹, —C(O)N(R³⁹)₂, —N(R³⁹)C(O)R³⁹, ═O, and —CN.

In some embodiments, R^{C} and R^{D} come together to form a 4- to 10-membered heterocycle optionally substituted with one or more substituents independently selected from 3 to 10-membered heterocycle optionally substituted with one or more substituents independently selected from C₁₋₆ alkyl.

In some embodiments, R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³¹, R³², R³⁴, R³⁵, R³⁶, R³⁸, and R³⁹ are each independently selected at each occurrence from: hydrogen, C₁₋₆ alkyl, C₁₋₆ haloalkyl, and C₁₋₆ hydroxyalkyl. In some embodiments, R³¹, R³², R³⁴, R³⁵, R³⁶, R³⁸, and R³⁹ are each independently selected at each occurrence from: hydrogen and C₁₋₆ alkyl. In some embodiments, R³¹, R³², R³⁴, R³⁵, R³⁶, R³⁸, and R³⁹ are each hydrogen. In some embodiments, R³¹, R³², R³⁴, R³⁵, R³⁶, R³⁸, and R³⁹ are each C₁₋₆ alkyl.

In some embodiments, R³¹ is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, R³¹ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³¹ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³¹ is independently selected at each occurrence from: hydrogen, C₁₋₆ alkyl, C₁₋₆ haloalkyl, and C₁₋₆ hydroxyalkyl. In some embodiments, R³¹ is independently selected at each occurrence from: hydrogen and C₁₋₆ alkyl. In some embodiments, each R³¹ is hydrogen. In some embodiments, each R³¹ is C₁₋₆ alkyl.

In some embodiments, R³¹ is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, R³¹ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³¹ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³¹ is independently selected at each occurrence from: hydrogen, C₁₋₆ alkyl, C₁₋₆ haloalkyl, and C₁₋₆ hydroxyalkyl. In some embodiments, R³¹ is independently selected at each occurrence from: hydrogen and C₁₋₆ alkyl. In some embodiments, each R³¹ is hydrogen. In some embodiments, each R³¹ is C₁₋₆ alkyl.

In some embodiments, R³² is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, R³² is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³² is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³² is independently selected at each occurrence from: hydrogen, C₁₋₆ alkyl, C₁₋₆ haloalkyl, and C₁₋₆ hydroxyalkyl. In some embodiments, R³² is independently selected at each occurrence from: hydrogen and C₁₋₆ alkyl. In some embodiments, each R³² is hydrogen. In some embodiments, each R³² is C₁₋₆ alkyl.

In some embodiments, R³⁴ is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, R³⁴ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³⁴ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³⁴ is independently selected at each occurrence from: hydrogen, C₁₋₆ alkyl, C₁₋₆ haloalkyl, and C₁₋₆ hydroxyalkyl. In some embodiments, R³⁴ is independently selected at each occurrence from: hydrogen and C₁₋₆ alkyl. In some embodiments, each R³⁴ is hydrogen. In some embodiments, each R³⁴ is C₁₋₆ alkyl.

In some embodiments, R³⁵ is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

In some embodiments, R³⁵ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³⁵ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³⁵ is independently selected at each occurrence from: hydrogen, C₁₋₆ alkyl, C₁₋₆ haloalkyl, and C₁₋₆ hydroxyalkyl. In some embodiments, R³⁵ is independently selected at each occurrence from: hydrogen and C₁₋₆ alkyl. In some embodiments, each R³⁵ is hydrogen. In some embodiments, each R³⁵ is C₁₋₆ alkyl.

In some embodiments, R³⁶ is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R³⁶ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³⁶ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³⁶ is independently selected at each occurrence from: hydrogen, C₁₋₆ alkyl, C₁₋₆ haloalkyl, and C₁₋₆ hydroxyalkyl. In some embodiments, R³⁶ is independently selected at each occurrence from: hydrogen and C₁₋₆ alkyl. In some embodiments, each R³⁶ is hydrogen. In some embodiments, each R³⁶ is C₁₋₆ alkyl.

In some embodiments, R³⁸ is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R³⁸ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³⁸ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³⁸ is independently selected at each occurrence from: hydrogen, C₁₋₆ alkyl, C₁₋₆ haloalkyl, and C₁₋₆ hydroxyalkyl. In some embodiments, R³⁸ is independently selected at each occurrence from: hydrogen and C₁₋₆ alkyl. In some embodiments, each R³⁸ is hydrogen. In some embodiments, each R³⁸ is C₁₋₆ alkyl.

In some embodiments, R³⁹ is independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy. In some embodiments, R³⁹ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy, C₃₋₆ carbocycle, and 3- to 6-membered heterocycle. In some embodiments, R³⁹ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R³⁹ is independently selected at each occurrence from: hydrogen, C₁₋₆ alkyl, C₁₋₆ haloalkyl, and C₁₋₆ hydroxyalkyl. In some embodiments, R³⁹ is independently selected at each occurrence from: hydrogen and C₁₋₆ alkyl. In some embodiments, each R³⁹ is hydrogen. In some embodiments, each R³⁹ is C₁₋₆ alkyl.

In some embodiments, the compound is a compound of Formula (IIIA):

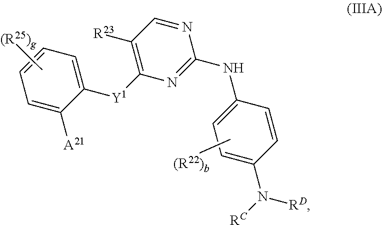

or a pharmaceutically acceptable salt thereof; wherein g is selected from 0, 1, 2, 3, and 4.

In some embodiments, the compound is a compound of Formula (IIIb):

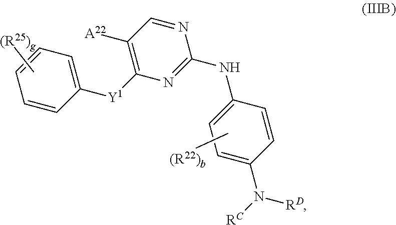

or a pharmaceutically acceptable salt thereof; wherein g is selected from 0, 1, 2, 3, and 4.

In some embodiments, R^{C} and R^{D} come together to form a 4- to 10-membered heterocycle optionally substituted with one or more substituents independently selected from:
halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
C₁₋₆ alkyl optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN; and
C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹ —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN.

In some embodiments, R^{C} and R^{D} come together to form a 4- to 10-membered heterocycle optionally substituted with one or more substituents independently selected from:
halogen, —OR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN;
C₁₋₆ alkyl optionally substituted with one or more substituents independently selected from: halogen, —OR³⁹, —N(R³⁹)₂, —C(O)R³⁹, —C(O)OR³⁹, —OC(O)R³⁹ —; OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN; and
C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —; C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), ═O, ═NR³⁹, —NO₂, and —CN.

In some embodiments, R^{C} and R^{D} come together to form a 4- to 10-membered heterocycle optionally substituted with one or more substituents independently selected from:
halogen, —OR³⁹, —N(R³⁹)₂, —C(O)R³⁹, —C(O)OR³⁹, —OC(O)R³⁹, —C(O)N(R³⁹)₂, —N(R³⁹)C(O)R³⁹, —N(R³⁹)S(O)₂(R³⁹), ═O, ═NR³⁹, and —CN;
C₁₋₆ alkyl optionally substituted with one or more substituents independently selected from: halogen, —OR³⁹, —N(R³⁹)₂, —C(O)R³⁹, —C(O)OR³⁹, —OC(O)R³⁹ —; C(O)N(R³⁹)₂, —N(R³⁹)C(O)R³⁹, —N(R³⁹)S(O)₂(R³⁹), ═O, and ═NR³⁹; and
C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —N(R³⁹)₂, —C(O)R³⁹ —C(O)OR³⁹, —OC(O)R³⁹, —C(O)N(R³⁹)₂, —N(R³⁹)C(O)R³⁹, —N(R³⁹)S(O)₂(R³⁹), ═O, ═NR³⁹, and —CN.

In some embodiments, R^{C} and R^{D} come together to form a 4- to 10-membered heterocycle optionally substituted with one or more substituents independently selected from: halogen, —OR³⁹, —N(R³⁹)₂, —C(O)R³⁹, —C(O)N(R³⁹)₂, —N(R³⁹)C(O)R³⁹, ═O, —CN, C₁₋₆ alkyl, and C₁₋₆ haloalkyl; and
C₃₋₁₀ carbocycle and 3- to 10-membered heterocycle, any of which is optionally substituted with one or more substituents independently selected from: halogen, C₁₋₆ alkyl, C₁₋₆ haloalkyl, —OR³⁹, —N(R³⁹)₂, —C(O)R³⁹, —C(O)N(R³⁹)₂, —N(R³⁹)C(O)R³⁹, ═O, and —CN.

In some embodiments, R^{C} and R^{D} come together to form a 4- to 10-membered heterocycle optionally substituted with one or more substituents independently selected from: halogen, C₁₋₆ alkyl, and C₁₋₆ haloalkyl, —OR³⁹, —N(R³⁹)₂, —C(O)R³⁹, —C(O)N(R³⁹)₂, —N(R³⁹)C(O)R³⁹, ═O, and —CN; and
C₃₋₁₀ carbocycle and 3- to 10-membered heterocycle, any of which is optionally substituted with one or more substituents independently selected from: halogen, C₁₋₆ alkyl, C₁₋₆ haloalkyl, —OR³⁹, —N(R³⁹)₂, —C(O)R³⁹, —C(O)N(R³⁹)₂, —N(R³⁹)C(O)R³⁹, ═O, and —CN.

In some embodiments, R^{C} and R^{D} come together to form a 4- to 10-membered heterocycle optionally substituted with one or more substituents independently selected from: halogen, C₁₋₆ alkyl, and C₁₋₆ haloalkyl, and 3- to 10-membered heterocycle, any of which is optionally substituted with one or more substituents independently selected from: halogen, C₁₋₆ alkyl, C₁₋₆ haloalkyl, —OR³⁹, —N(R³⁹)₂, —C(O)R³⁹, —C(O)N(R³⁹)₂, —N(R³⁹)C(O)R³⁹, ═O, and —CN.

In some embodiments, R^{C} and R^{D} come together to form a 4- to 10-membered heterocycle optionally substituted with one or more substituents independently selected from 3- to 10-membered heterocycle optionally substituted with one or more substituents independently selected from C₁₋₆ alkyl. In some embodiments, R^{C} and R^{D} come together to form a 4- to 10-membered heterocycle optionally substituted with one or more substituents independently selected from: halogen, C₁₋₆ alkyl, and C₁₋₆ haloalkyl, and 5- to 6-membered heterocycle, wherein the 5- to 6-membered heterocycle is optionally substituted with one or more substituents independently selected from: halogen, C₁₋₆ alkyl, and C₁₋₆ haloalkyl. In some embodiments, R^{C} and R^{D} come together to form a 4- to 10-membered heterocycle optionally substituted with one or more substituents independently selected from 5- to 6-membered heterocycle optionally substituted with one or more substituents independently selected from C₁₋₆ alkyl.

In some embodiments, the compound is a compound of Formula (IVA):

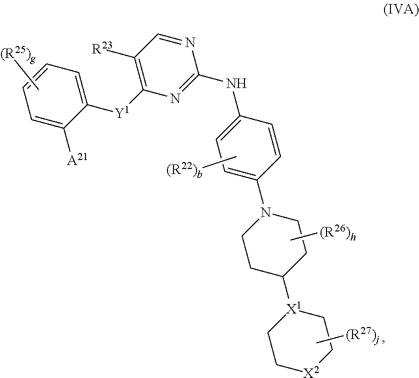

or a pharmaceutically acceptable salt thereof; wherein
X¹ is selected from C(R²⁸) and N;
X² is selected from C(R²⁸)₂ and N(R²⁹);
R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹, —C(O)OR³⁹ —; OC(O)R³⁹, —OC(O)N(R³⁹)₂, —C(O)N(R³⁹)₂, —N(R³⁹)C(O)R³⁹, —N(R³⁹)C(O)OR³⁹1 —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R²⁹ is selected from C₁₋₆ alkyl, C₁₋₆ haloalkyl, and C₁₋₆ hydroxyalkyl;
h is selected from 0, 1, 2, 3, 4, 5, 6, 7, and 8; and
j is selected from 0, 1, 2, 3, 4, 5, 6, 7, 8, and 9.

In some embodiments, the compound is a compound of Formula (IVB):

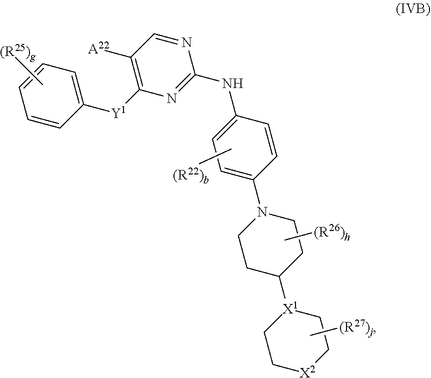

or a pharmaceutically acceptable salt thereof; wherein
X¹ is selected from C(R²⁸) and N;
X² is selected from C(R²⁸)₂ and N(R²⁹);
R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹, —C(O)OR³⁹ —; OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R²⁹ is selected from C₁₋₆ alkyl, C₁₋₆ haloalkyl, and C₁₋₆ hydroxyalkyl;
h is selected from 0, 1, 2, 3, 4, 5, 6, 7, and 8; and
j is selected from 0, 1, 2, 3, 4, 5, 6, 7, 8, and 9.

In some embodiments, X¹ is selected from C(R²⁸) and N. In some embodiments, X¹ is C(R²⁸). In some embodiments, X¹ is N. In some embodiments, X² is C(R²⁸)₂. In some embodiments, X² is N(R²⁹). In some embodiments, X¹ is N; and X² is N(R²⁹).

In some embodiments, h is selected from 0, 1, 2, 3, 4, 5, 6, 7, and 8. In some embodiments, h is selected from 0, 1, 2, 3, and 4. In some embodiments, h is selected from 0, 1, 2, and 3. In some embodiments, h is selected from 0, 1, 2, 3, and 4. In some embodiments, h is selected from 0, 1, and 2. In some embodiments, h is selected from 0 and 1. In some embodiments, h is 8. In some embodiments, h is 7. In some embodiments, h is 6. In some embodiments, h is 5. In some embodiments, h is 4. In some embodiments, h is 3. In some embodiments, h is 2. In some embodiments, h is 1. In some embodiments, h is 0.

In some embodiments, j is selected from 0, 1, 2, 3, 4, 5, 6, 7, 8, and 9. In some embodiments, j is selected from 0, 1, 2, 3, and 4. In some embodiments, j is selected from 0, 1, 2, and 3. In some embodiments, j is selected from 0, 1, 2, 3, and 4. In some embodiments, j is selected from 0, 1, and 2. In some embodiments, j is selected from 0 and 1. In some embodiments, j is 9. In some embodiments, j is 8. In some embodiments, j is 7. In some embodiments, j is 6. In some embodiments, j is 5. In some embodiments, j is 4. In some embodiments, j is 3. In some embodiments, j is 2. In some embodiments, j is 1. In some embodiments, j is 0.

In some embodiments, R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN. In some embodiments, R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), and ═O. In some embodiments, R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹ —C(O)OR³⁹, —OC(O)R³⁹, —C(O)N(R³⁹)₂, —N(R³⁹)C(O)R³⁹, —N(R³⁹)S(O)₂(R³⁹), and ═O. In some embodiments, R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, and ═O. In some embodiments, R²⁶, R²⁷, and R²⁸ are each independently selected at each occurrence from C₁₋₆ alkyl.

In some embodiments, R²⁹ is selected from C₁₋₆ alkyl, C₁₋₆ haloalkyl, and C₁₋₆ hydroxyalkyl. In some embodiments, R²⁹ is selected from C₁₋₆ alkyl and C₁₋₆ haloalkyl. In some embodiments, R²⁹ is selected from C₁₋₆ alkyl and C₁₋₆ hydroxyalkyl. In some embodiments, R²⁹ is selected from C₁₋₆ haloalkyl and C₁₋₆ hydroxyalkyl. In some embodiments, R²⁹ is C₁₋₆ haloalkyl. In some embodiments, R²⁹ is C₁₋₆ hydroxyalkyl. In some embodiments, R²⁹ is C₁₋₆ alkyl. In some embodiments, R²⁹ is methyl.

In some embodiments, the compound is a compound of Formula (VA):

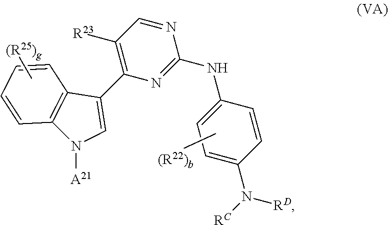

or a pharmaceutically acceptable salt thereof.

In some embodiments, the compound is a compound of Formula (VB):

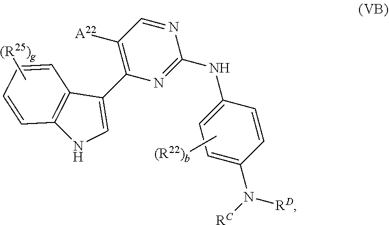

or a pharmaceutically acceptable salt thereof.

In some embodiments, R^{C} and R^{D} are each independently selected from: hydrogen and C₁₋₆ alkyl optionally substituted with one or more substituents independently selected from: halogen, —OR³⁸, —SR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), —S(O)R³⁸, —S(O)₂R³⁸, —S(O)₂N(R³⁸)₂, ═O, ═S, ═NR³⁸, —NO₂, and —CN. In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), ═O, ═NR³⁸, —NO₂, and —CN. In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)OR³⁸, —OC(O)R³⁸, —C(O)N(R³⁸)₂, —N(R³⁸)C(O)R³⁸, —N(R³⁸)S(O)₂(R³⁸), ═O, ═NR³⁸, —NO₂, and —CN. In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)N(R³⁸)₂, —N(R³⁸)C(O)R³⁸, ═O, and —CN. In some embodiments, R^{C} and R^{D} are each independently selected from: hydrogen and C₁₋₆ alkyl optionally substituted with one or more substituents independently selected from: halogen, —OR³⁸, —N(R³⁸)₂, —C(O)R³⁸, —C(O)N(R³⁸)₂, —N(R³⁸)C(O)R³⁸, and —CN. In some embodiments, R^{C} and R^{D} are each independently selected from hydrogen and C₁₋₆ alkyl optionally substituted with one or more substituents independently selected from —N(R³⁸)₂. In some embodiments, R^{C} and R^{D} are each independently selected from C₁₋₆ alkyl optionally substituted with one or more substituents independently selected from —N(R³⁸)₂. In some embodiments, R^{C} and R^{D} are each independently selected from C₁₋₆ alkyl optionally substituted with one or more substituents independently selected from —N(R³⁸)₂, wherein R³⁸ is selected from C₁₋₄ alkyl. In some embodiments, R^{C} and R^{D} are each independently selected from methyl and

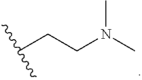 [SMILES: *CCN(C)C]

In some embodiments, g is selected from 0, 1, 2, 3, and 4. In some embodiments, g is selected from 0, 1, 2, and 3. In some embodiments, g is selected from 0, 1, and 2. In some embodiments, g is selected from 0 and 1. In some embodiments, g is 0. In some embodiments, g is 1. In some embodiments, g is 2. In some embodiments, g is 3. In some embodiments, g is 4.

In some embodiments, the compound is selected from the compounds of Table 1, or a pharmaceutically acceptable salt thereof.

TABLE 1

| Cmpd. | |
|---|---|
| No. | Structure |
| 3 | 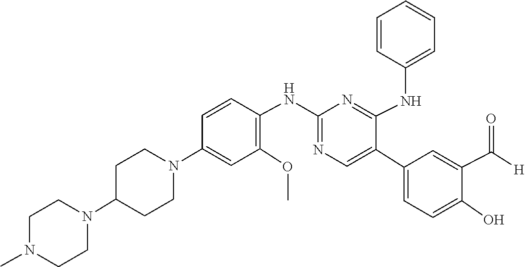 [SMILES: COc1cc(N2CCC(N3CCN(C)CC3)CC2)ccc1Nc1ncc(-c2ccc(O)c(C=O)c2)c(Nc2ccccc2)n1] |
| 4 | 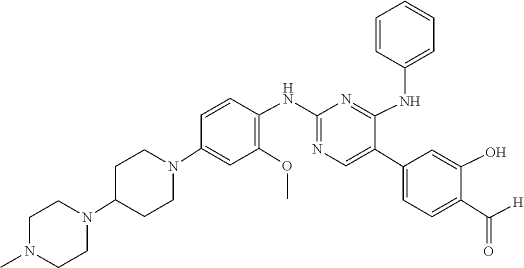 [SMILES: COc1cc(N2CCC(N3CCN(C)CC3)CC2)ccc1Nc1ncc(-c2ccc(C=O)c(O)c2)c(Nc2ccccc2)n1] |
| 5 | 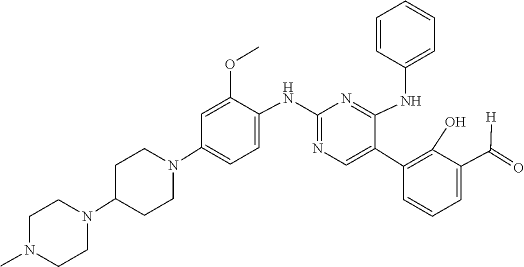 [SMILES: COc1cc(N2CCC(N3CCN(C)CC3)CC2)ccc1Nc1ncc(-c2cccc(C=O)c2O)c(Nc2ccccc2)n1] |
| 6 | 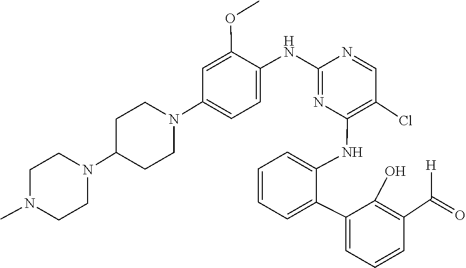 [SMILES: COc1cc(N2CCC(N3CCN(C)CC3)CC2)ccc1Nc1ncc(Cl)c(Nc2ccccc2-c2cccc(C=O)c2O)n1] |
| 7 | 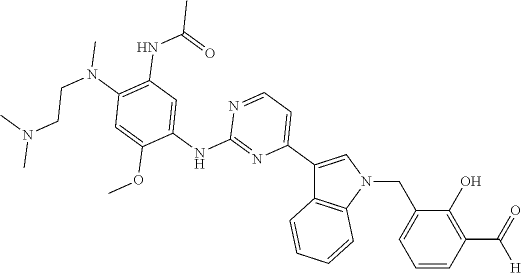 [SMILES: COc1cc(N(C)CCN(C)C)c(NC(C)=O)cc1Nc1nccc(-c2cn(Cc3cccc(C=O)c3O)c3ccccc23)n1] |
| 8 | 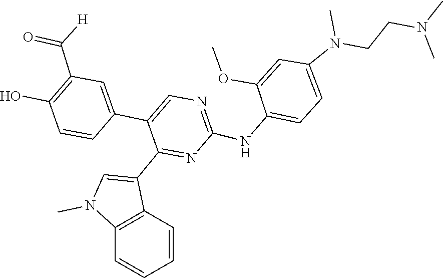 [SMILES: COc1cc(N(C)CCN(C)C)ccc1Nc1ncc(-c2ccc(O)c(C=O)c2)c(-c2cn(C)c3ccccc23)n1] |
| 9 | 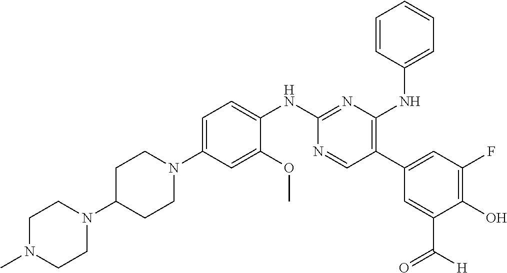 [SMILES: COc1cc(N2CCC(N3CCN(C)CC3)CC2)ccc1Nc1ncc(-c2cc(F)c(O)c(C=O)c2)c(Nc2ccccc2)n1] |
| 10 | 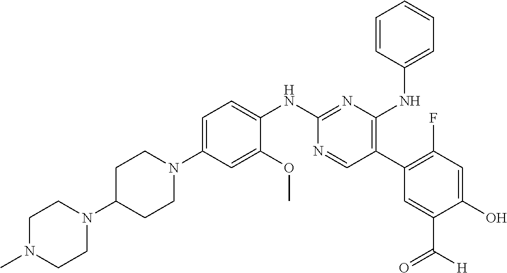 [SMILES: COc1cc(N2CCC(N3CCN(C)CC3)CC2)ccc1Nc1ncc(-c2cc(C=O)c(O)cc2F)c(Nc2ccccc2)n1] |
| 11 | 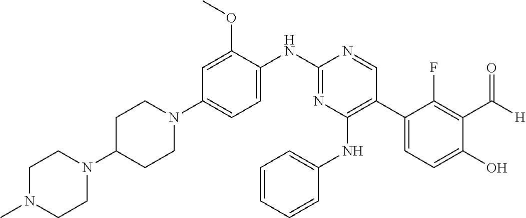 [SMILES: COc1cc(N2CCC(N3CCN(C)CC3)CC2)ccc1Nc1ncc(-c2ccc(O)c(C=O)c2F)c(Nc2ccccc2)n1] |
| 12 | 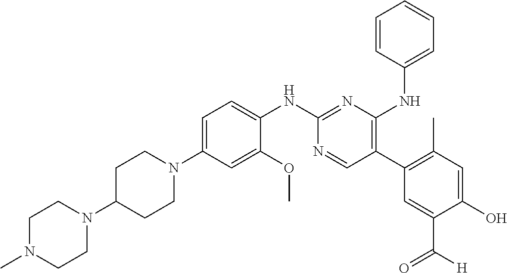 [SMILES: COc1cc(N2CCC(N3CCN(C)CC3)CC2)ccc1Nc1ncc(-c2cc(C=O)c(O)cc2C)c(Nc2ccccc2)n1] |
| 13 | 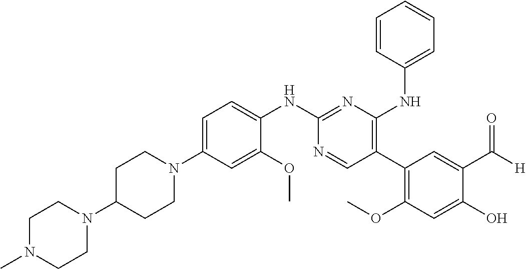 [SMILES: COc1cc(N2CCC(N3CCN(C)CC3)CC2)ccc1Nc1ncc(-c2cc(C=O)c(O)cc2OC)c(Nc2ccccc2)n1] |
| 14 | 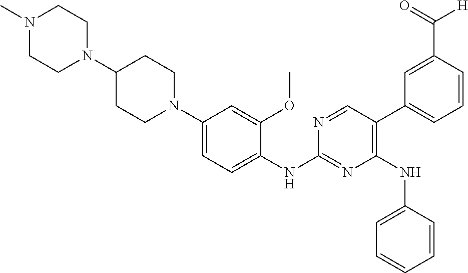 [SMILES: COc1cc(N2CCC(N3CCN(C)CC3)CC2)ccc1Nc1ncc(-c2cccc(C=O)c2)c(Nc2ccccc2)n1] |
| 15 | 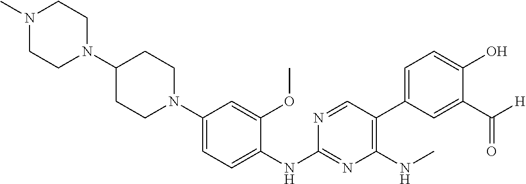 [SMILES: CNc1nc(Nc2ccc(N3CCC(N4CCN(C)CC4)CC3)cc2OC)ncc1-c1ccc(O)c(C=O)c1] |
| 16 | 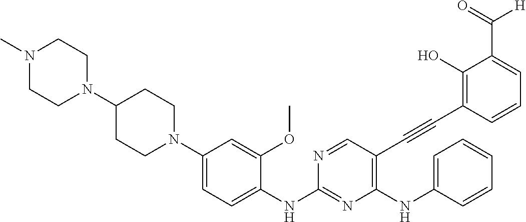 [SMILES: COc1cc(N2CCC(N3CCN(C)CC3)CC2)ccc1Nc1ncc(C#Cc2cccc(C=O)c2O)c(Nc2ccccc2)n1] |
| 17 | 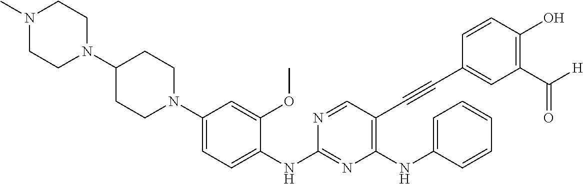 [SMILES: COc1cc(N2CCC(N3CCN(C)CC3)CC2)ccc1Nc1ncc(C#Cc2ccc(O)c(C=O)c2)c(Nc2ccccc2)n1] |
| 18 | 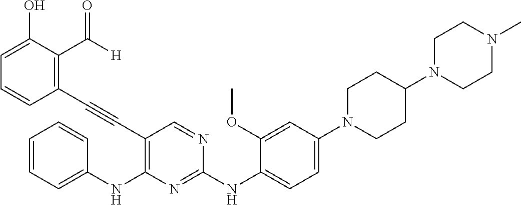 [SMILES: COc1cc(N2CCC(N3CCN(C)CC3)CC2)ccc1Nc1ncc(C#Cc2cccc(O)c2C=O)c(Nc2ccccc2)n1] |
| 19 | 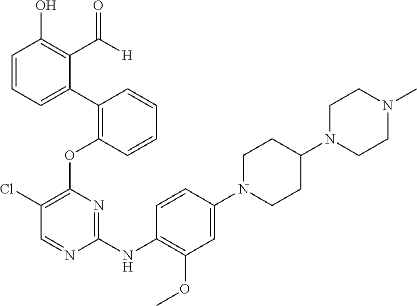 [SMILES: COc1cc(N2CCC(N3CCN(C)CC3)CC2)ccc1Nc1ncc(Cl)c(Oc2ccccc2-c2cccc(O)c2C=O)n1] |
| 20 | 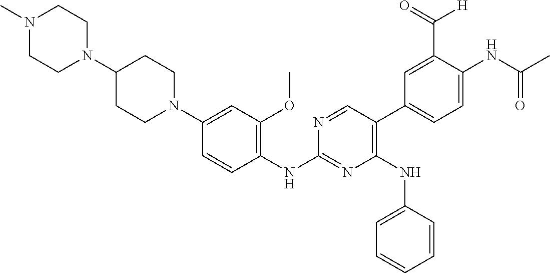 [SMILES: COc1cc(N2CCC(N3CCN(C)CC3)CC2)ccc1Nc1ncc(-c2ccc(NC(C)=O)c(C=O)c2)c(Nc2ccccc2)n1] |
| 21 | 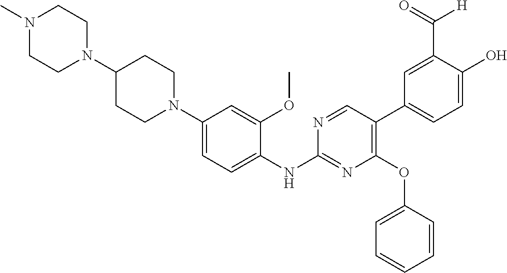 [SMILES: COc1cc(N2CCC(N3CCN(C)CC3)CC2)ccc1Nc1ncc(-c2ccc(O)c(C=O)c2)c(Oc2ccccc2)n1] |
| 22 | 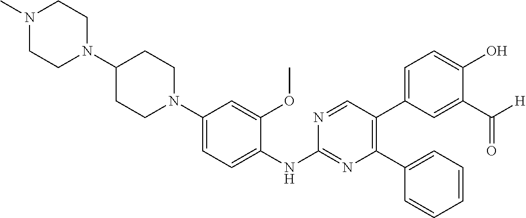 [SMILES: COc1cc(N2CCC(N3CCN(C)CC3)CC2)ccc1Nc1ncc(-c2ccc(O)c(C=O)c2)c(-c2ccccc2)n1] |
| 23 | 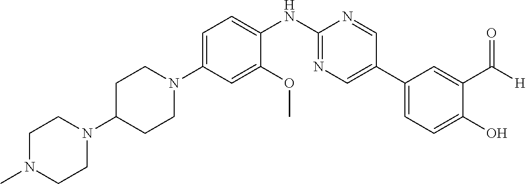 [SMILES: COc1cc(N2CCC(N3CCN(C)CC3)CC2)ccc1Nc1ncc(-c2ccc(O)c(C=O)c2)cn1] |
| 24 | 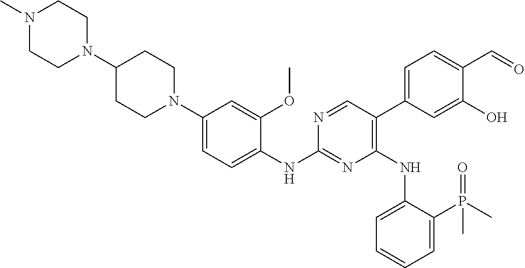 [SMILES: COc1cc(N2CCC(N3CCN(C)CC3)CC2)ccc1Nc1ncc(-c2ccc(C=O)c(O)c2)c(Nc2ccccc2P(C)(C)=O)n1] |
| 25 | 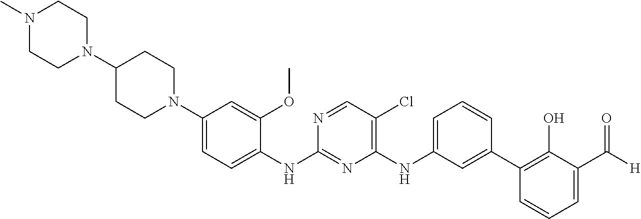 [SMILES: COc1cc(N2CCC(N3CCN(C)CC3)CC2)ccc1Nc1ncc(Cl)c(Nc2cccc(-c3cccc(C=O)c3O)c2)n1] |
| 26 | 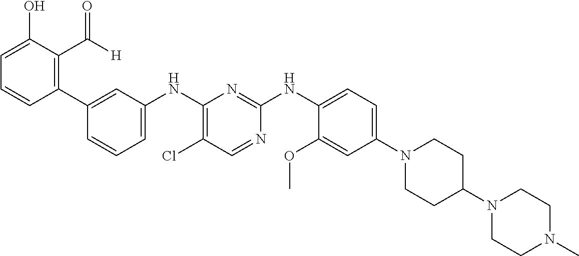 [SMILES: COc1cc(N2CCC(N3CCN(C)CC3)CC2)ccc1Nc1ncc(Cl)c(Nc2cccc(-c3cccc(O)c3C=O)c2)n1] |
| 27 | 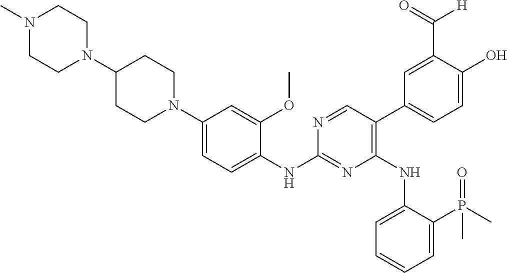 |
| 28 | 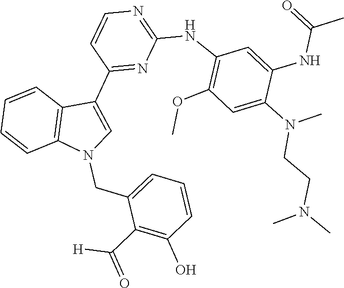 [SMILES: COc1cc(N(C)CCN(C)C)c(NC(C)=O)cc1Nc1nccc(-c2cn(Cc3cccc(O)c3C=O)c3ccccc23)n1] |
| 29 | 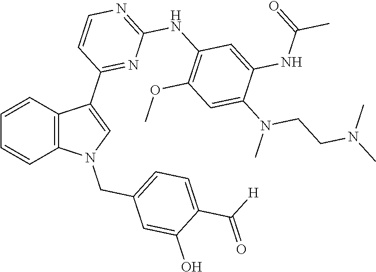 [SMILES: COc1cc(N(C)CCN(C)C)c(NC(C)=O)cc1Nc1nccc(-c2cn(Cc3ccc(C=O)c(O)c3)c3ccccc23)n1] |
| 30 | 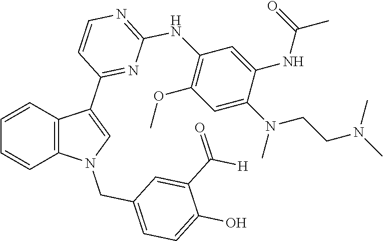 [SMILES: COc1cc(N(C)CCN(C)C)c(NC(C)=O)cc1Nc1nccc(-c2cn(Cc3ccc(O)c(C=O)c3)c3ccccc23)n1] |
| 31 | 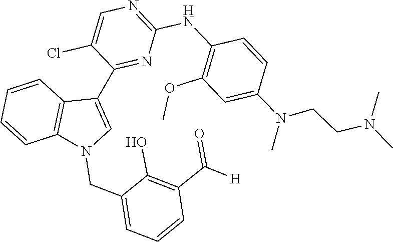 [SMILES: COc1cc(N(C)CCN(C)C)ccc1Nc1ncc(Cl)c(-c2cn(Cc3cccc(C=O)c3O)c3ccccc23)n1] |
| 32 | 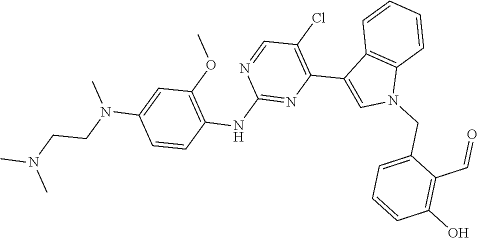 [SMILES: COc1cc(N(C)CCN(C)C)ccc1Nc1ncc(Cl)c(-c2cn(Cc3cccc(O)c3C=O)c3ccccc23)n1] |
| 33 | 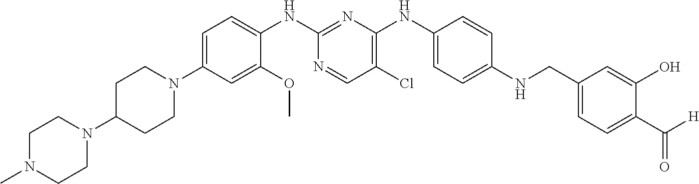 [SMILES: COc1cc(N2CCC(N3CCN(C)CC3)CC2)ccc1Nc1ncc(Cl)c(Nc2ccc(NCc3ccc(C=O)c(O)c3)cc2)n1] |
| 34 | 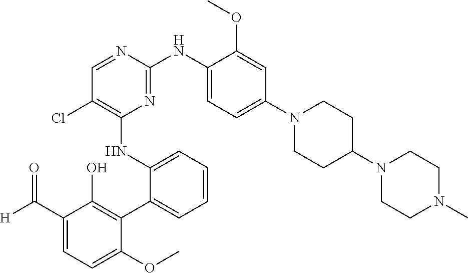 [SMILES: COc1cc(N2CCC(N3CCN(C)CC3)CC2)ccc1Nc1ncc(Cl)c(Nc2ccccc2-c2c(OC)ccc(C=O)c2O)n1] |
| 35 | 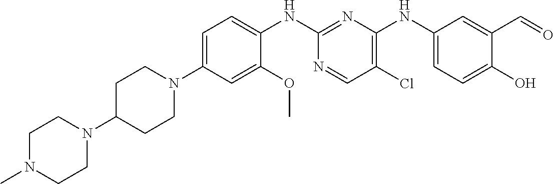 [SMILES: COc1cc(N2CCC(N3CCN(C)CC3)CC2)ccc1Nc1ncc(Cl)c(Nc2ccc(O)c(C=O)c2)n1] |

Table 1: Cmpd.

Structure No.

In some aspects, the present disclosure provides compounds represented by the structure of Formula (VI):

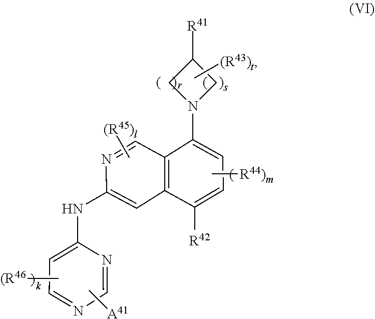

or a pharmaceutically acceptable salt thereof, wherein:
A⁴¹ is represented by -L⁴¹-C⁴¹;
   L⁴¹ is selected from a bond and -L^{C}-L^{D}-;
      L^{C} is selected from absent, —O—, —S—, and —N(R⁴⁷)—; and
      L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹, —C(O)OR⁵¹ —OC(O)R⁵¹, —OC(O)N(R⁵¹)₂, —C(O)N(R⁵¹)₂, —N(R⁵¹)C(O)R⁵¹, —N(R⁵¹)C(O)OR⁵¹, —N(R⁵¹)C(O)N(R⁵¹)₂, —N(R⁵¹)S(O)₂(R⁵¹), —S(O)R⁵¹, —S(O)₂R⁵¹, —S(O)₂N(R⁵¹)₂, ═O, ═S, ═NR⁵¹, —NO₂, and —CN;
   C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —C(O)R⁵³, —C(O)OR⁵², —OC(O)R⁵², —OC(O)N(R⁵²)₂, —C(O)N(R⁵²)₂, —N(R⁵²)C(O)R⁵², —N(R⁵²)C(O)OR⁵², —N(R⁵²)C(O)N(R⁵²)₂, —N(R⁵²)S(O)₂(R⁵²), —S(O)R⁵², —S(O)₂R⁵², —S(O)₂N(R⁵²)₂, —NO₂, and —CN;
R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —OC(O)N(R⁵⁴)₂, —C(O)N(R⁵⁴)₂, —N(R⁵⁴)C(O)R⁵⁴, —N(R⁵⁴)C(O)OR⁵⁴, —N(R⁵⁴)C(O)N(R⁵⁴)₂, —N(R⁵⁴)S(O)₂(R⁵⁴), —S(O)R⁵⁴, —S(O)₂R⁵⁴, —S(O)₂N(R⁵⁴)₂, ═O, ═S, ═NR⁵⁴, —NO₂, and —CN;
R⁴² is C₁₋₆ alkyl optionally substituted with one or more substituent independently selected at each occurrence from: halogen, —OR⁵⁵, —SR⁵⁵, —N(R⁵⁵)₂, —C(O)R⁵⁵, —C(O)OR⁵⁵ —OC(O)R⁵⁵, —OC(O)N(R⁵⁵)₂, —C(O)N(R⁵⁵)₂, —N(R⁵⁵)C(O)R⁵⁵, —N(R⁵⁵)C(O)OR⁵⁵, —N(R⁵⁵)C(O)N(R⁵⁵)₂, —N(R⁵⁵)S(O)₂(R⁵⁵), —S(O)R⁵⁵, —S(O)₂R⁵⁵, —S(O)₂N(R⁵⁵)₂, ═O, ═S, ═NR⁵⁵, —NO₂, and —CN;
R⁴³ is selected from halogen, C₁₋₆ alkyl, C₁₋₆ haloalkyl, —OR⁵⁶, —SR⁵⁶, —N(R⁵⁶)₂, —C(O)R⁵⁶, —C(O)OR⁵⁶, —OC(O)R⁵⁶, —OC(O)N(R⁵⁶)₂, —C(O)N(R⁵⁶)₂, —N(R⁵⁶)C(O)R⁵⁶, —N(R⁵⁶)C(O)OR⁵⁶, —N(R⁵⁶)C(O)N(R⁵⁶)₂, —N(R⁵⁶)S(O)₂(R⁵⁶), —S(O)R⁵⁶, —S(O)₂R⁵⁶, —S(O)₂N(R⁵⁶)₂, —NO₂, and —CN; or two R⁴³ attached to the same carbon atom together form ═O, ═S, and ═NR⁵⁶;
R⁴⁴, R⁴⁵, and R⁴⁶ are each independently selected at each occurrence from: halogen, C₁₋₆ alkyl, C₁₋₆ haloalkyl, —OR⁵⁷, —SR⁵⁷, —N(R⁵⁷)₂, —C(O)R⁷, —C(O)OR⁷, —OC(O)R⁵⁷, —OC(O)N(R⁵⁷)₂, —C(O)N(R⁵⁷)₂, —N(R⁵⁷)C(O)R⁵⁷, —N(R⁵⁷)C(O)OR⁵⁷, —N(R⁵⁷)C(O)N(R⁵⁷)₂, —N(R⁵⁷)S(O)₂(R⁵⁷), —S(O)R⁵⁷, —S(O)₂R⁵⁷, —S(O)₂N(R⁵⁷)₂, —NO₂, and —CN;
R⁴⁷ is selected from hydrogen, C₁₋₆ alkyl, and C₁₋₆ haloalkyl;
R⁵¹, R⁵², R⁵⁴, R⁵⁵, R⁵⁶, and R⁵⁷ are each independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy; and R⁵³ is selected at each occurrence from: C₁₋₆ alkyl, C₁₋₆ haloalkyl, C₁₋₆ hydroxyalkyl, C₁₋₆ alkoxy, and C₁₋₆ haloalkoxy;
r and s are each independently selected from 1 and 2;
t is selected from 0, 1, 2, 3, and 4; and
k, l, and m are each independently selected from 0, 1, and 2.

In some embodiments, the present disclosure provides compounds represented by the structure of Formula (VI):

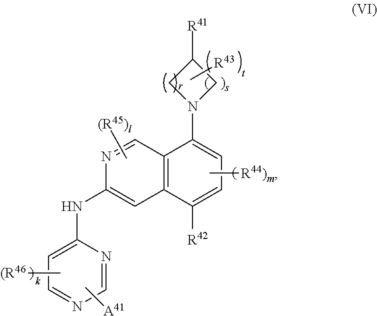

or a pharmaceutically acceptable salt thereof, wherein:
A⁴¹ is represented by -L⁴¹-C⁴¹;
   L⁴¹ is selected from a bond and -L^{C}-L^{D}-;
      L^{C} is selected from absent, —O—, —S—, and —N(R⁴⁷)—; and
      L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹, —C(O)OR⁵¹ —OC(O)R⁵¹, —OC(O)N(R⁵¹)₂, —C(O)N(R⁵¹)₂, —N(R⁵¹)C(O)R⁵¹, —N(R⁵¹)C(O)OR⁵¹, —N(R⁵¹)C(O)N(R⁵¹)₂, —N(R⁵¹)S(O)₂(R⁵¹), ═O, ═NR⁵¹, —NO₂, and —CN;
   C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —C(O)R⁵³, —C(O)OR⁵², —OC(O)R⁵², —OC(O)N(R⁵²)₂, —C(O)N(R⁵²)₂, —N(R⁵²)C(O)R⁵², —N(R⁵²)C(O)OR⁵², —N(R⁵²)C(O)N(R⁵²)₂, —N(R⁵²)S(O)₂(R⁵²), —NO₂, and —CN;
R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —OC(O)N(R⁵⁴)₂, —C(O)N(R⁵⁴)₂, —N(R⁵⁴)C(O)R⁵⁴, —N(R⁵⁴)C(O)OR⁵⁴, —N(R⁵⁴)C(O)N(R⁵⁴)₂, —N(R⁵⁴)S(O)₂(R⁵⁴), —S(O)R⁵⁴, —S(O)₂R⁵⁴ —S(O)₂N(R⁵⁴)₂, ═O, ═S, ═NR⁵⁴, —NO₂, and —CN;
R⁴² is C₁₋₆ alkyl optionally substituted with one or more substituent independently selected at each occurrence from: halogen, —OR⁵⁵, —SR⁵⁵, —N(R⁵⁵)₂, —C(O)R⁵⁵, —C(O)OR⁵⁵ —OC(O)R⁵⁵, —OC(O)N(R⁵⁵)₂, —C(O)N(R⁵⁵)₂, —N(R⁵⁵)C(O)R⁵⁵, —N(R⁵⁵)C(O)OR¹¹, —N(R⁵⁵)C(O)N(R⁵⁵)₂, —N(R⁵⁵)S(O)₂(R⁵⁵), ═O, ═S, ═NR11, —NO₂, and —CN;
R⁴³ is selected from halogen, C₁₋₆ alkyl, C₁₋₆ haloalkyl, —OR⁵⁶, —SR⁵⁶, —N(R⁵⁶)₂, —C(O)R⁵⁶, —C(O)OR⁵⁶, —OC(O)R⁵⁶, —OC(O)N(R⁵⁶)₂, —C(O)N(R⁵⁶)₂, —N(R⁵⁶)C(O)R⁵⁶, —N(R⁵⁶)C(O)OR⁵⁶, —N(R⁵⁶)C(O)N(R¹⁶)₂, —N(R⁵⁶)S(O)₂(R⁵⁶), —S(O)R⁵⁶, —S(O)₂R⁵⁶, —S(O)₂N(R⁵⁶)₂, —NO₂, and —CN; or
two R⁴³ attached to the same carbon atom together form ═O, ═S, and ═NR⁵⁶;
R⁴⁴, R⁴⁵, and R⁴⁶ are each independently selected at each occurrence from: halogen, C₁₋₆ alkyl, C₁₋₆ haloalkyl, —OR⁵⁷, —SR⁵⁷, —N(R⁵⁷)₂, —C(O)R^{S7}, —C(O)OR^{S7}, —OC(O)R^{S7}, —OC(O)N(R⁵⁷)₂, —C(O)N(R⁵⁷)₂, —N(R⁵⁷)C(O)R⁵⁷, —N(R⁵⁷)C(O)OR⁵⁷, —N(R⁵⁷)C(O)N(R⁵⁷)₂, —N(R⁵⁷)S(O)₂(R⁵⁷), —NO₂, and —CN;
R⁴⁷ is selected from hydrogen, C₁₋₆ alkyl, and C₁₋₆ haloalkyl;
R⁵¹, R⁵², R⁵⁴, R⁵⁵, R⁵⁶, and R⁵⁷ are each independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy; and R⁵³ is selected at each occurrence from: C₁₋₆ alkyl, C₁₋₆ haloalkyl, C₁₋₆ hydroxyalkyl, C₁₋₆ alkoxy, and C₁₋₆ haloalkoxy;
r and s are each independently selected from 1 and 2;
t is selected from 0, 1, 2, 3, and 4; and
k, l, and m are each independently selected from 0, 1, and 2.

In some embodiments, the present disclosure provides compounds represented by the structure of Formula (VI):

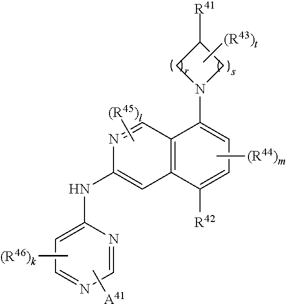

or a pharmaceutically acceptable salt thereof, wherein:
A⁴¹ is represented by -L⁴¹-C⁴¹;
   L⁴¹ is selected from a bond and -L^{C}-L^{D}-;
      L^{C} is selected from absent, —O—, —S—, and —N(R⁴⁷)—; and
      L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹, —C(O)OR⁵¹ —OC(O)R⁵¹, —C(O)N(R⁵¹)₂, —N(R⁵¹)C(O)R⁵¹, —N(R⁵¹)S(O)₂(R⁵¹), ═O, —NO₂, and —CN;
   C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —C(O)R⁵³, —C(O)OR⁵², —OC(O)R⁵², —C(O)N(R⁵²)₂, —N(R⁵²)C(O)R⁵², —N(R⁵²)S(O)₂(R⁵²), —NO₂, and —CN;
R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —C(O)N(R⁵⁴)₂, —N(R⁵⁴)C(O)R⁵⁴, —N(R⁵⁴)S(O)₂(R⁵⁴), —S(O)R⁵⁴, —S(O)₂R⁵⁴, —S(O)₂N(R⁵⁴)₂, and ═O;
R⁴² is C₁₋₆ alkyl optionally substituted with one or more substituent independently selected at each occurrence from: halogen, —OR⁵⁵, —SR⁵⁵, —N(R⁵⁵)₂, —C(O)R⁵⁵, —C(O)OR⁵⁵ —OC(O)R⁵⁵, —C(O)N(R⁵⁵)₂, —N(R⁵⁵)C(O)R⁵⁵, —N(R⁵⁵)S(O)₂(R⁵⁵), and ═O;
R⁴³ is selected from halogen, C₁₋₆ alkyl, C₁₋₆ haloalkyl, —OR⁵⁶, —SR⁵⁶, —N(R⁵⁶)₂, —C(O)R⁵⁶, —C(O)OR⁵⁶, —OC(O)R⁵⁶, —C(O)N(R⁵⁶)₂, —N(R⁵⁶)C(O)R⁵⁶, —N(R⁵⁶)S(O)₂(R⁵⁶), —S(O)R⁵⁶, —S(O)₂R⁵⁶, and —S(O)₂N(R⁵⁶)₂; or
two R⁴³ attached to the same carbon atom together form ═O;
R⁴⁴, R⁴⁵, and R⁴⁶ are each independently selected at each occurrence from: halogen, C₁₋₆ alkyl, C₁₋₆ haloalkyl, —OR⁵⁷, —SR⁵⁷, —N(R⁵⁷)₂, —C(O)R⁵⁷, —C(O)OR⁵⁷, —OC(O)R⁵⁷, —C(O)N(R⁵⁷)₂, —N(R⁵⁷)C(O)R⁵⁷, —N(R⁵⁷)S(O)₂(R⁵⁷), —NO₂, and —CN;
R⁴⁷ is selected from hydrogen, C₁₋₆ alkyl, and C₁₋₆ haloalkyl;
R⁵¹, R⁵², R⁵⁴, R⁵⁵, R⁵⁶, and R⁵⁷ are each independently selected at each occurrence from: hydrogen, C₁₋₆ alkyl, C₁₋₆ haloalkyl, and C₁₋₆ hydroxyalkyl; and
R⁵³ is selected at each occurrence from: C₁₋₆ alkyl, C₁₋₆ haloalkyl, and C₁₋₆ hydroxyalkyl;
r and s are each independently selected from 1 and 2;
t is selected from 0, 1, 2, 3, and 4; and
k, l, and m are each independently selected from 0, 1, and 2.

In some embodiments, the present disclosure provides compounds represented by the structure of Formula (VI-A):

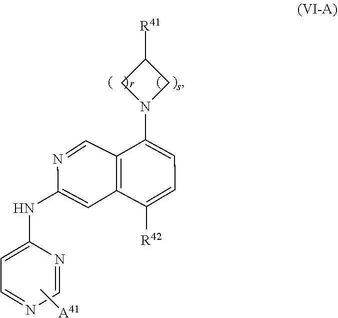

or a pharmaceutically acceptable salt thereof, wherein:
A⁴¹ is represented by -L⁴¹-C⁴¹;
   L⁴¹ is selected from a bond and -L^{C}-L^{D}-;
      L^{C} is selected from absent, —O—, and —N(R⁴⁷)—; and
      L^{D} is selected from absent, C₁₋₄ alkylene, C₂₋₄ alkynylene, phenylene, and 5- to 6-membered heterocyclene, any of which is optionally substituted with one or more substituents independently selected from: halogen, —OR⁵¹, —SR⁵¹ —N(R⁵¹)₂, —C(O)R⁵¹, —C(O)OR⁵¹, —OC(O)R⁵¹, —C(O)N(R⁵¹)₂, —N(R⁵¹)C(O)R⁵¹, —N(R⁵¹)S(O)₂(R⁵¹), ═O, —NO₂, and —CN;
   C⁴¹ is selected from phenyl substituted with —C(O)H and optionally substituted with one or more substituents independently selected from: halogen, —OR⁵², —SR⁵² —; N(R⁵²)₂, —C(O)R⁵³, —C(O)OR⁵², —OC(O)R⁵², —C(O)N(R⁵²)₂, —N(R⁵²)C(O)R⁵², —N(R⁵²)S(O)₂(R⁵²), —NO₂, and —CN;
R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —C(O)N(R⁵⁴)₂, —N(R⁵⁴)C(O)R⁵⁴, —N(R⁵⁴)S(O)₂(R⁵⁴), —S(O)R⁵⁴, —S(O)₂R⁵⁴, —S(O)₂N(R⁵⁴)₂, and ═O;
R⁴² is C₁₋₆ alkyl optionally substituted with one or more substituent independently selected at each occurrence from: halogen, —OR⁵⁵, —SR⁵⁵, —N(R⁵⁵)₂, —C(O)R⁵⁵, —C(O)OR⁵⁵ —OC(O)R⁵⁵, —C(O)N(R⁵⁵)₂, —N(R⁵⁵)C(O)R⁵⁵, —N(R⁵⁵)S(O)₂(R⁵⁵), and ═O;
R⁴⁷ is selected from hydrogen and C₁₋₆ alkyl;
R⁵¹, R⁵², R⁵⁴, and R⁵⁵ are each independently selected at each occurrence from: hydrogen, C₁₋₆ alkyl, C₁₋₆ haloalkyl, and C₁₋₆ hydroxyalkyl; and
R⁵³ is selected at each occurrence from C₁₋₆ alkyl;
r and s are each 1.

In some embodiments, the present disclosure provides compounds represented by the structure of Formula (VI-A):

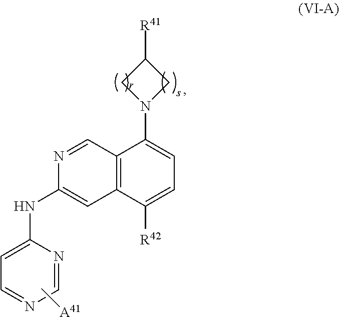

or a pharmaceutically acceptable salt thereof, wherein:
A⁴¹ is represented by -L⁴¹-C⁴¹;
   L⁴¹ is selected from a bond and -L^{C}-L^{D}-;
      L^{C} is selected from absent, —O—, and —N(R⁴⁷)—; and
      L^{D} is selected from absent, C₁₋₄ alkylene, C₂₋₄ alkynylene, phenylene, and 5- to 6-membered heterocyclene;
   C⁴¹ is selected from phenyl substituted with —C(O)H and optionally substituted with one or more substituents independently selected from: halogen and —OR⁵²;
R⁴¹ is C₁₋₄ alkyl optionally substituted with one or more substituents independently selected at each occurrence from —S(O)₂R⁵⁴;
R⁴² is C₁₋₆ alkyl;
R⁴⁷ is selected from hydrogen and C₁₋₆ alkyl;
R⁵⁴ is independently selected at each occurrence from C₁₋₆ alkyl; and
r and s are each 1.

In some embodiments, R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —OC(O)N(R⁵⁴)₂, —C(O)N(R⁵⁴)₂, —N(R⁵⁴)C(O)R⁵⁴, —N(R⁵⁴)C(O)OR⁵⁴, —N(R⁵⁴)C(O)N(R⁵⁴)₂, —N(R⁵⁴)S(O)₂(R⁵⁴), —S(O)R⁵⁴, —S(O)₂R⁵⁴, —S(O)₂N(R⁵⁴)₂, ═O, ═S, ═NR⁵⁴, —NO₂, and —CN. In some embodiments, R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —C(O)N(R⁵⁴)₂, —N(R⁵⁴)C(O)R⁵⁴, —N(R⁵⁴)S(O)₂(R⁵⁴), —S(O)R⁵⁴, —S(O)₂R⁵⁴, —S(O)₂N(R⁵⁴)₂, and ═O. In some embodiments, R⁴¹ is selected from C₁₋₄ alkyl optionally substituted with one or more substituent independently selected at each occurrence from: —N(R⁵⁴)S(O)₂(R⁵⁴), —S(O)R⁵⁴, —S(O)₂R⁵⁴, and —S(O)₂N(R⁵⁴)₂. In some embodiments, R⁴¹ is selected from C₁₋₄ alkyl optionally substituted with one or more substituent independently selected at each occurrence from: —S(O)R⁵⁴, —S(O)₂R⁵⁴, and —S(O)₂N(R⁵⁴)₂. In some embodiments, R⁴¹ is selected from C₁₋₄ alkyl optionally substituted with one or more substituent independently selected at each occurrence from —S(O)₂R¹⁴.

In some embodiments, R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —OC(O)N(R⁵⁴)₂, —C(O)N(R⁵⁴)₂, —N(R⁵⁴)C(O)R⁵⁴, —N(R⁵⁴)C(O)OR⁵⁴, —N(R⁵⁴)C(O)N(R⁵⁴)₂, —N(R⁵⁴)S(O)₂(R⁵⁴), —S(O)R⁵⁴, —S(O)₂R⁵⁴, —S(O)₂N(R⁵⁴)₂, ═O, ═S, ═NR⁵⁴, —NO₂, and —CN; and R⁵⁴ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —C(O)N(R⁵⁴)₂, —N(R⁵⁴)C(O)R⁵⁴, —N(R⁵⁴)S(O)₂(R⁵⁴), —S(O)R⁵⁴, —S(O)₂R⁵⁴, —S(O)₂N(R⁵⁴)₂, and ═O; and R⁵⁴ is independently selected at each occurrence from: hydrogen, C₁₋₆ alkyl, C₁₋₆ haloalkyl, and C₁₋₆ hydroxyalkyl. In some embodiments, R⁴¹ is selected from C₁₋₄ alkyl optionally substituted with one or more substituent independently selected at each occurrence from: —N(R⁵⁴)S(O)₂(R⁵⁴), —S(O)R⁵⁴, —S(O)₂R⁵⁴, and —S(O)₂N(R⁵⁴)₂; and R⁵⁴ is each independently selected at each occurrence from hydrogen and C₁₋₆ alkyl. In some embodiments, R⁴¹ is selected from C₁₋₄ alkyl optionally substituted with one or more substituent independently selected at each occurrence from: —N(R⁵⁴)S(O)₂(R⁵⁴), —S(O)R⁵⁴, —S(O)₂R⁵⁴, and —S(O)₂N(R⁵⁴)₂; and R⁵⁴ is selected at each occurrence from: hydrogen, C₁₋₄ alkyl, C₁₄ haloalkyl, and C₁₄ hydroxyalkyl. In some embodiments, R⁴¹ is selected from C₁₋₄ alkyl optionally substituted with one or more substituent independently selected at each occurrence from: —S(O)R⁵⁴, —S(O)₂R⁵⁴, and —S(O)₂N(R⁵⁴)₂; and R⁵⁴ is independently selected at each occurrence from hydrogen and methyl. In some embodiments, R⁴¹ is selected from C₁₋₄ alkyl optionally substituted with one or more substituent independently selected at each occurrence from —S(O)R⁵⁴ and —S(O)₂R⁵⁴; and R⁵⁴ is independently selected at each occurrence from hydrogen and methyl. In some embodiments, R⁴¹ is selected from C₁₋₄ alkyl optionally substituted with one or more substituent independently selected at each occurrence from —S(O)₂R⁵⁴; and R⁵⁴ is independently selected at each occurrence from hydrogen and methyl. In some embodiments, R⁴¹ is selected from C₁₋₄ alkyl optionally substituted with one or more substituent independently selected at each occurrence from —S(O)₂R⁵⁴; and each R⁵⁴ is hydrogen. In some embodiments, R⁴¹ is selected from C₁₋₄ alkyl optionally substituted with one or more substituent independently selected at each occurrence from —S(O)₂R⁵⁴; and each R⁵⁴ is methyl. In some embodiments, R⁴¹ is

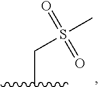 [SMILES: *CS(C)(=O)=O]

In some embodiments, R⁴² is C₁₋₆ alkyl optionally substituted with one or more substituent independently selected at each occurrence from: halogen, —OR⁵⁵, —SR⁵⁵ —N(R⁵⁵)₂, —C(O)R⁵⁵, —C(O)OR⁵⁵, —OC(O)R⁵⁵, —OC(O)N(R⁵⁵)₂, —C(O)N(R⁵⁵)₂, —N(R⁵⁵)C(O)R⁵⁵, —N(R⁵⁵)C(O)OR⁵⁵, —N(R⁵⁵)C(O)N(R⁵⁵)₂, —N(R⁵⁵)S(O)₂(R⁵⁵), —S(O)R⁵⁵, —S(O)₂R⁵⁵, —S(O)₂N(R⁵⁵)₂, ═O, ═S, ═NR⁵⁵, —NO₂, and —CN. In some embodiments, R⁴² is C₁₋₆ alkyl optionally substituted with one or more substituent independently selected at each occurrence from: halogen, —OR⁵⁵ —SR⁵⁵ —N(R⁵⁵)₂, —C(O)R⁵⁵, —C(O)OR⁵⁵, —OC(O)R⁵⁵, —OC(O)N(R⁵⁵)₂, —C(O)N(R⁵⁵)₂, —N(R⁵⁵)C(O)R⁵⁵, —N(R⁵⁵)C(O)OR⁵⁵, —N(R⁵⁵)C(O)N(R⁵⁵)₂, —N(R⁵⁵)S(O)₂(R⁵⁵), ═O, ═S, ═NR⁵⁵, —NO₂, and —CN. In some embodiments, R⁴² is C₁₋₆ alkyl optionally substituted with one or more substituent independently selected at each occurrence from: halogen, —OR⁵⁵, —SR⁵⁵, —N(R⁵⁵)₂, —C(O)R⁵⁵, —C(O)OR⁵⁵, —OC(O)R⁵⁵, —C(O)N(R⁵⁵)₂, —N(R⁵⁵)C(O)R⁵⁵, —N(R⁵⁵)S(O)₂(R⁵⁵), and ═O.

In some embodiments, R⁴² is C₁₋₆ alkyl optionally substituted with one or more substituent independently selected at each occurrence from: halogen, —OR⁵⁵, —N(R⁵⁵)₂, —C(O)R⁵⁵ —C(O)OR⁵⁵, —OC(O)R⁵⁵, —C(O)N(R⁵⁵)₂, and —N(R⁵⁵)C(O)R⁵⁵, and ═O. In some embodiments, R⁴² is C₁₋₆ alkyl optionally substituted with one or more substituent independently selected at each occurrence from: halogen, —OR⁵⁵ and —N(R⁵⁵)₂. In some embodiments, R⁴² is C₁₋₆ alkyl. In some embodiments, R⁴² is propyl. In some embodiments, R⁴² is 2-propyl.

In some embodiments, L⁴¹ is selected from a bond and -L^{C}-L^{D}-, wherein:
L^{C} is selected from absent, —O—, —S—, and —N(R⁴⁷)—; and
L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹ —C(O)OR⁵¹, —OC(O)R⁵¹, —OC(O)N(R⁵¹)₂, —C(O)N(R⁵¹)₂, —N(R⁵¹)C(O)R⁵¹, —N(R⁵¹)C(O)OR⁵¹, —N(R⁵¹)C(O)N(R⁵¹)₂, —N(R⁵¹)S(O)₂(R⁵¹), —S(O)R⁵¹, —S(O)₂R⁵¹ —S(O)₂N(R⁵¹)₂, ═O, ═S, ═NR⁵¹, —NO₂, and —CN.

In some embodiments, L⁴¹ is selected from a bond and -L^{C}-L^{D}-, wherein:
L^{C} is selected from absent, —O—, —S—, and —N(R⁴⁷)—; and
L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹ —C(O)OR⁵¹, —OC(O)R⁵¹, —OC(O)N(R⁵¹)₂, —C(O)N(R⁵¹)₂, —N(R⁵¹)C(O)R⁵¹, —N(R⁵¹)C(O)OR⁵¹, —N(R⁵¹)C(O)N(R⁵¹)₂, —N(R⁵¹)S(O)₂(R⁵¹), ═O, ═NR⁵¹, —NO₂, and —CN.

In some embodiments, L⁴¹ is selected from a bond and -L^{C}-L^{D}-, wherein:
L^{C} is selected from absent, —O—, —S—, and —N(R⁴⁷)—; and
L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹ —C(O)OR⁵¹, —OC(O)R⁵¹, —C(O)N(R⁵¹)₂, —N(R⁵¹)C(O)R⁵¹, —N(R⁵¹)S(O)₂(R⁵¹), ═O, —NO₂, and —CN.

In some embodiments, L⁴¹ is selected from a bond and -L^{C}-L^{D}-, wherein:
L^{C} is selected from absent, —O—, and —N(R⁴⁷)—; and
L^{D} is selected from absent, C₁₋₄ alkylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹, —C(O)OR⁵¹ —OC(O)R⁵¹, —C(O)N(R⁵¹)₂, —N(R⁵¹)C(O)R⁵¹, —N(R⁵¹)S(O)₂(R⁵¹), ═O, —NO₂, and —CN.

In some embodiments, L⁴¹ is selected from a bond and -L^{C}-L^{D}-, wherein:
L^{C} is selected from absent, —O—, and —N(R⁴⁷)—; and
L^{D} is selected from absent, C₁₋₄ alkylene, C₂₋₄ alkynylene, phenylene, and 5- to 6-membered heterocyclene.

In some embodiments, L⁴¹ is a bond. In some embodiments, L⁴¹ is -L^{C}-L^{D}-.

In some embodiments, L^{C} is selected from absent, —O—, —S—, and —N(R⁴⁷)—. In some embodiments, L^{C} is selected from absent, —O—, and —N(R⁴⁷)—. In some embodiments, L^{C} is selected from absent and —O—. In some embodiments, L^{C} is selected from absent and —N(R⁴⁷)—. In some embodiments, L^{C} is selected from —O— and —N(R⁴⁷)—. In some embodiments, L^{C} is absent. In some embodiments, L^{C} is —O—. In some embodiments, L^{C} is —N(R⁴⁷)—.

In some embodiments, L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹ —N(R⁵¹)₂, —C(O)R⁵¹, —C(O)OR⁵¹, —OC(O)R⁵¹, —OC(O)N(R⁵¹)₂, —C(O)N(R⁵¹)₂, —N(R⁵¹)C(O)R⁵¹, —N(R⁵¹)C(O)OR⁵¹, —N(R⁵¹)C(O)N(R⁵¹)₂, —N(R⁵¹)S(O)₂(R⁵¹), —S(O)R⁵¹, —S(O)₂R⁵¹, —S(O)₂N(R⁵¹)₂, ═O, ═S, ═NR⁵¹, —NO₂, and —CN. In some embodiments, L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹ —N(R⁵¹)₂, —C(O)R⁵¹, —C(O)OR⁵¹, —OC(O)R⁵¹, —OC(O)N(R⁵¹)₂, —C(O)N(R⁵¹)₂, —N(R⁵¹)C(O)R⁵¹, —N(R⁵¹)C(O)OR⁵¹, —N(R⁵¹)C(O)N(R⁵¹)₂, —N(R⁵¹)S(O)₂(R⁵¹), ═O, ═NR⁵¹, —NO₂, and —CN. In some embodiments, L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹, —C(O)OR⁵¹, —OC(O)R⁵¹, —C(O)N(R⁵¹)₂, —N(R⁵¹)C(O)R⁵¹, —N(R⁵¹)S(O)₂(R⁵¹), ═O, —NO₂, and —CN.

In some embodiments, L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹ —N(R⁵¹)₂, —C(O)R⁵¹, —C(O)OR⁵¹, —OC(O)R⁵¹, —C(O)N(R⁵¹)₂, —N(R⁵¹)C(O)R⁵¹, ═O, and —CN. In some embodiments, L^{D} is selected from absent, C₁₋₄ alkylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹ —C(O)OR⁵¹, —OC(O)R⁵¹, —C(O)N(R⁵¹)₂, —N(R⁵¹)C(O)R⁵¹, —N(R⁵¹)S(O)₂(R⁵¹), ═O, —NO₂, and —CN.

In some embodiments, L^{D} is selected from absent, C₁₋₄ alkylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹ —C(O)OR⁵¹, —OC(O)R⁵¹, —C(O)N(R⁵¹)₂, —N(R⁵¹)C(O)R⁵¹, ═O, and —CN. In some embodiments, L^{D} is selected from absent, C₁₋₄ alkylene, C₂₋₄ alkynylene, phenylene, and 5- to 6-membered heterocyclene, any of which is optionally substituted with one or more substituents independently selected from: halogen, —OR⁵¹, —SR⁵¹ —N(R⁵¹)₂, and ═O. In some embodiments, L^{D} is selected from absent, C₁₋₄ alkylene, C₂₋₄ alkynylene, phenylene, and pyrazolene, any of which is optionally substituted with one or more substituents independently selected from: halogen, —OR⁵¹, —SR⁵¹ —N(R⁵¹)₂, and ═O. In some embodiments, L^{D} is selected from absent, C₁₄ alkylene, C₂₋₄ alkynylene, phenylene, and 5- to 6-membered heterocyclene. In some embodiments, L^{D} is selected from absent, methylene, ethylene, ethynylene, phenylene, and pyrazolene.

In some embodiments, L⁴¹ is selected from a bond, —O—, —OCH₂—, —N(CH₃)CH₂—, —N(CH₃)CH₂CH₂—, ethynylene,

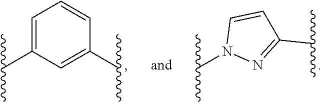 [SMILES: *c1cccc(*)c1 ; *c1ccn(*)n1]

In some embodiments, C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —C(O)R⁵³ —; C(O)OR⁵², —OC(O)R¹², —OC(O)N(R¹²)₂, —C(O)N(R¹²)₂, —N(R⁵²)C(O)R¹², —N(R⁵²)C(O)OR¹², —N(R⁵²)C(O)N(R⁵²)₂, —N(R⁵²)S(O)₂(R¹²), —S(O)R¹², —S(O)₂R¹², —S(O)₂N(R¹²)₂, —NO₂, and —CN. In some embodiments, C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —C(O)R⁵³ —; C(O)OR⁵², —OC(O)R⁵², —OC(O)N(R⁵²)₂, —C(O)N(R⁵²)₂, —N(R⁵²)C(O)R⁵², —N(R⁵²)C(O)OR⁵², —N(R⁵²)C(O)N(R⁵²)₂, —N(R⁵²)S(O)₂(R⁵²), —NO₂, and —CN. In some embodiments, C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —C(O)R⁵³, —C(O)OR⁵², —OC(O)R⁵², —C(O)N(R⁵²)₂, —N(R⁵²)C(O)R⁵², —N(R⁵²)S(O)₂(R⁵²), —NO₂, and —CN. In some embodiments, C⁴¹ is selected from phenyl substituted with —C(O)H and optionally substituted with one or more substituents independently selected from: halogen, —OR⁵², —SR⁵², —N(R⁵²)₂, —C(O)R⁵³, —C(O)OR⁵², —OC(O)R⁵², —C(O)N(R⁵²)₂, —N(R⁵²)C(O)R⁵², —N(R⁵²)S(O)₂(R⁵²), —NO₂, and —CN. In some embodiments, C⁴¹ is selected from phenyl and monocyclic 5- to 6-membered heteroaryl, any of which is substituted with —C(O)H and optionally substituted with one or more substituents independently selected from: halogen, —OR⁵², —SR⁵², —N(R⁵²)₂, —N(R⁵²)C(O)R⁵², —N(R⁵²)C(O)OR⁵², —N(R⁵²)C(O)N(R⁵²)₂, —N(R⁵²)S(O)₂(R⁵²), —NO₂, and —CN. In some embodiments, C⁴¹ is selected from phenyl substituted with —C(O)H and optionally substituted with one or more substituents independently selected from: halogen and —OR⁵². In some embodiments, C⁴¹ is selected from phenyl and monocyclic 5- to 6-membered heteroaryl, any of which is substituted with —C(O)H and —OH, and optionally substituted with one or more substituents independently selected from: halogen, and —OR⁵², —SR⁵², —N(R⁵²)₂, —N(R⁵²)C(O)R⁵², —N(R⁵²)C(O)OR⁵², —N(R⁵²)C(O)N(R⁵²)₂, —N(R⁵²)S(O)₂(R⁵²), —NO₂, and —CN. In some embodiments, C⁴¹ is selected from phenyl and monocyclic 5- to 6-membered heteroaryl, any of which is substituted with —C(O)H and —OH, and optionally substituted with one or more substituents independently selected from halogen.

In some embodiments, C⁴¹ is selected from:

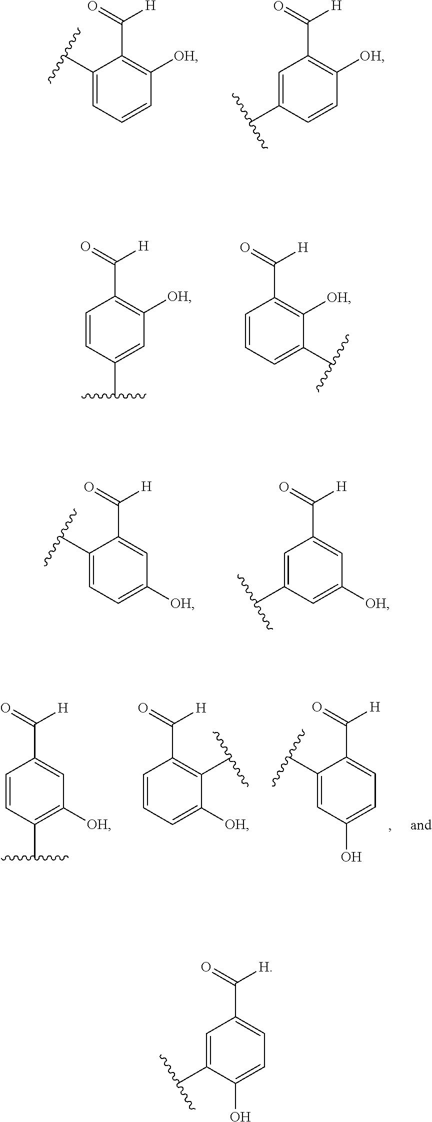

In some embodiments, C⁴¹ is selected from:

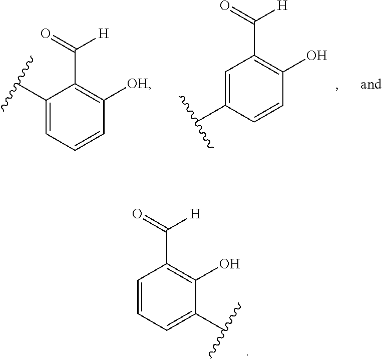 [SMILES: *c1cccc(O)c1C=O ; *c1ccc(O)c(C=O)c1 ; *c1cccc(C=O)c1O]

In some embodiments, r and s are each independently selected from 1 and 2. In some embodiments, r and s are each 1. In some embodiments, r and s are each 2. In some embodiments, r is selected from 1 and 2. In some embodiments, r is 1. In some embodiments, r is 2. In some embodiments, s is selected from 1 and 2. In some embodiments, s is 1. In some embodiments, s is 2.

In some embodiments, t is selected from 0, 1, 2, 3, and 4. In some embodiments, t is selected from 0, 1, 2, and 3. In some embodiments, t is selected from 0, 1, and 2. In some embodiments, t is selected from 0 and 1. In some embodiments, t is 0. In some embodiments, t is 1. In some embodiments, t is 2. In some embodiments, t is 3. In some embodiments, t is 4.

In some embodiments, k, l, and m are each independently selected from 0, 1, and 2. In some embodiments, k, l, and m are each independently selected from 0 and 1. In some embodiments, k, l, and m are each 0. In some embodiments, k, l, and m are each 1. In some embodiments, k, l, and m are each 2. In some embodiments, k is selected from 0, 1, and 2. In some embodiments, k is selected from 0 and 1. In some embodiments, k is 0. In some embodiments, k is 1. In some embodiments, k is 2. In some embodiments, 1 is selected from 0, 1, and 2. In some embodiments, 1 is selected from 0 and 1. In some embodiments, 1 is 0. In some embodiments, 1 is 1. In some embodiments, 1 is 2. In some embodiments, m is selected from 0, 1, and 2. In some embodiments, m is selected from 0 and 1. In some embodiments, m is 0. In some embodiments, m is 1. In some embodiments, m is 2.

In some embodiments, R⁵¹, R⁵², R⁵⁴, R⁵⁵, R⁵⁶, and R⁵⁷ are each independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁵¹, R⁵², R⁵⁴, R⁵⁵, R⁵⁶, and R⁵⁷ are each independently selected at each occurrence from: hydrogen, C₁₋₆ alkyl, C₁₋₆ haloalkyl, and C₁₋₆ hydroxyalkyl. In some embodiments, R⁵¹, R⁵², R⁵⁴, R⁵⁵, R⁵⁶, and R⁵⁷ are each independently selected at each occurrence from hydrogen and C₁₋₆ alkyl. In some embodiments, R⁵¹, R⁵², R⁵⁴, R⁵⁵, R⁵⁶, and R⁵⁷ are each independently selected at each occurrence from hydrogen and methyl. In some embodiments, R⁵¹, R⁵², R⁵⁴, R⁵⁵, R⁵⁶, and R⁵⁷ are each independently selected at each occurrence from hydrogen. In some embodiments, R⁵¹, R⁵², R⁵⁴, R⁵⁵, R⁵⁶, and R⁵⁷ are each independently selected at each occurrence from methyl.

In some embodiments, R⁵¹ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁵¹ is independently selected at each occurrence from: hydrogen, C₁₋₆ alkyl, C₁₋₆ haloalkyl, and C₁₋₆ hydroxyalkyl. In some embodiments, R⁵¹ is each independently selected at each occurrence from hydrogen and C₁₋₆ alkyl. In some embodiments, R⁵¹ is independently selected at each occurrence from hydrogen and methyl. In some embodiments, R⁵¹ is independently selected at each occurrence from hydrogen. In some embodiments, R⁵¹ is independently selected at each occurrence from methyl.

In some embodiments, R⁵² is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁵² is independently selected at each occurrence from: hydrogen, C₁₋₆ alkyl, C₁₋₆ haloalkyl, and C₁₋₆ hydroxyalkyl. In some embodiments, R⁵² is each independently selected at each occurrence from hydrogen and C₁₋₆ alkyl. In some embodiments, R⁵² is independently selected at each occurrence from hydrogen and methyl. In some embodiments, R⁵² is independently selected at each occurrence from hydrogen. In some embodiments, R⁵² is independently selected at each occurrence from methyl.

In some embodiments, R⁵⁴ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁵⁴ is independently selected at each occurrence from: hydrogen, C₁₋₆ alkyl, C₁₋₆ haloalkyl, and C₁₋₆ hydroxyalkyl. In some embodiments, R⁵⁴ is each independently selected at each occurrence from hydrogen and C₁₋₆ alkyl. In some embodiments, R⁵⁴ is independently selected at each occurrence from hydrogen and methyl. In some embodiments, R⁵⁴ is independently selected at each occurrence from hydrogen. In some embodiments, R⁵⁴ is independently selected at each occurrence from methyl.

In some embodiments, R⁵⁵ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁵⁵ is independently selected at each occurrence from: hydrogen, C₁₋₆ alkyl, C₁₋₆ haloalkyl, and C₁₋₆ hydroxyalkyl. In some embodiments, R⁵⁵ is each independently selected at each occurrence from hydrogen and C₁₋₆ alkyl. In some embodiments, R⁵⁵ is independently selected at each occurrence from hydrogen and methyl. In some embodiments, R⁵⁵ is independently selected at each occurrence from hydrogen. In some embodiments, R⁵⁵ is independently selected at each occurrence from methyl.

In some embodiments, R⁵⁶ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁵⁶ is independently selected at each occurrence from: hydrogen, C₁₋₆ alkyl, C₁₋₆ haloalkyl, and C₁₋₆ hydroxyalkyl. In some embodiments, R⁵⁶ is each independently selected at each occurrence from hydrogen and C₁₋₆ alkyl. In some embodiments, R⁵⁶ is independently selected at each occurrence from hydrogen and methyl. In some embodiments, R⁵⁶ is independently selected at each occurrence from hydrogen. In some embodiments, R⁵⁶ is independently selected at each occurrence from methyl.

In some embodiments, R⁵⁷ is independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy. In some embodiments, R⁵⁷ is independently selected at each occurrence from: hydrogen, C₁₋₆ alkyl, C₁₋₆ haloalkyl, and C₁₋₆ hydroxyalkyl. In some embodiments, R⁵⁷ is each independently selected at each occurrence from hydrogen and C₁₋₆ alkyl. In some embodiments, R⁵⁷ is independently selected at each occurrence from hydrogen and methyl. In some embodiments, R⁵⁷ is independently selected at each occurrence from hydrogen. In some embodiments, R⁵⁷ is independently selected at each occurrence from methyl.

In some embodiments, the compound is selected from a compound of Table 2, or a pharmaceutically acceptable salt thereof.

TABLE 2

| Cmpd. No. | Structure |
|---|---|
| 41 | 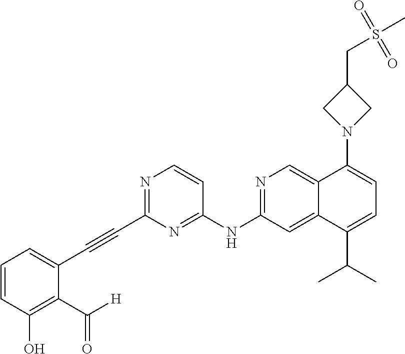 [SMILES: CC(C)c1ccc(N2CC(CS(C)(=O)=O)C2)c2cnc(Nc3ccnc(C#Cc4cccc(O)c4C=O)n3)cc12] |
| 42 | 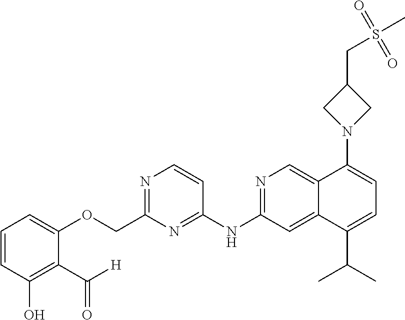 [SMILES: CC(C)c1ccc(N2CC(CS(C)(=O)=O)C2)c2cnc(Nc3ccnc(COc4cccc(O)c4C=O)n3)cc12] |
| 43 | 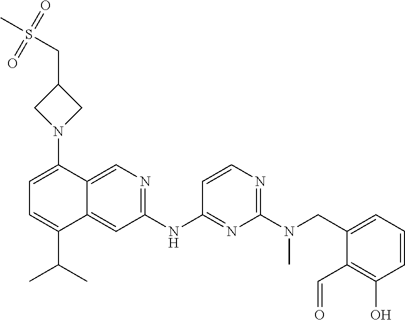 [SMILES: CC(C)c1ccc(N2CC(CS(C)(=O)=O)C2)c2cnc(Nc3ccnc(N(C)Cc4cccc(O)c4C=O)n3)cc12] |
| 44 | 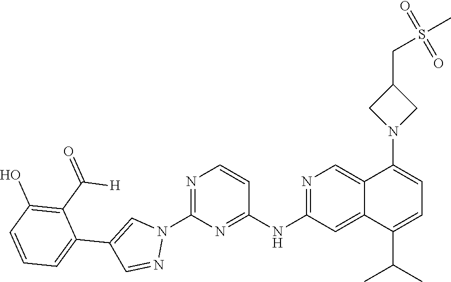 [SMILES: CC(C)c1ccc(N2CC(CS(C)(=O)=O)C2)c2cnc(Nc3ccnc(-n4cc(-c5cccc(O)c5C=O)cn4)n3)cc12] |
| 45 | 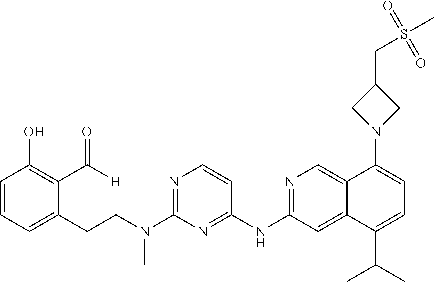 [SMILES: CC(C)c1ccc(N2CC(CS(C)(=O)=O)C2)c2cnc(Nc3ccnc(N(C)CCc4cccc(O)c4C=O)n3)cc12] |
| 46 | 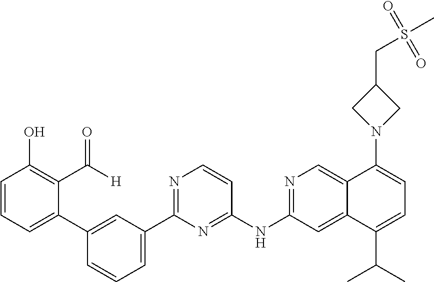 [SMILES: CC(C)c1ccc(N2CC(CS(C)(=O)=O)C2)c2cnc(Nc3ccnc(-c4cccc(-c5cccc(O)c5C=O)c4)n3)cc12] |
| 47 | 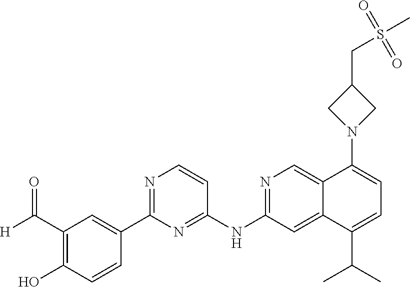 [SMILES: CC(C)c1ccc(N2CC(CS(C)(=O)=O)C2)c2cnc(Nc3ccnc(-c4ccc(O)c(C=O)c4)n3)cc12] |
| 48 | 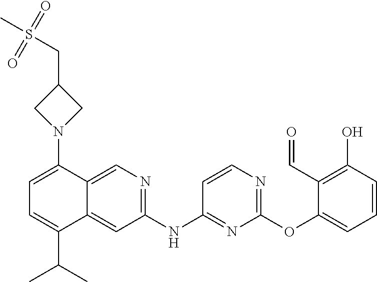 [SMILES: CC(C)c1ccc(N2CC(CS(C)(=O)=O)C2)c2cnc(Nc3ccnc(Oc4cccc(O)c4C=O)n3)cc12] |
| 49 | 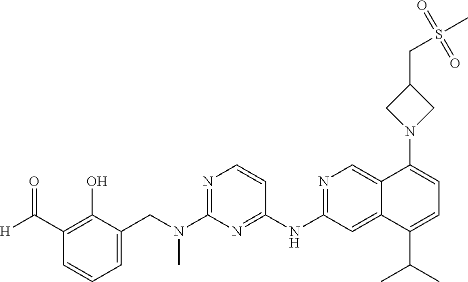 [SMILES: CC(C)c1ccc(N2CC(CS(C)(=O)=O)C2)c2cnc(Nc3ccnc(N(C)Cc4cccc(C=O)c4O)n3)cc12] |
| 50 | 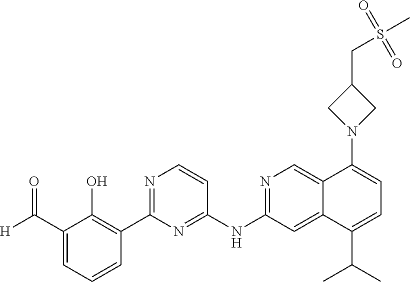 [SMILES: CC(C)c1ccc(N2CC(CS(C)(=O)=O)C2)c2cnc(Nc3ccnc(-c4cccc(C=O)c4O)n3)cc12] |

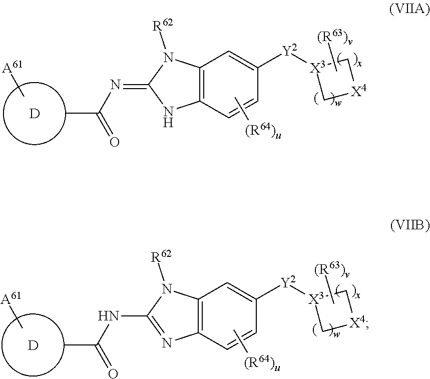

or a pharmaceutically acceptable salt thereof, wherein:
D is a phenyl or a 5- to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —OC(O)N(R⁷⁰)₂, —C(O)N(R⁷⁰)₂, —N(R⁷⁰)C(O)R⁷⁰, —N(R⁷⁰)C(O)OR⁷⁰, —N(R⁷⁰)C(O)N(R⁷⁰)₂, —N(R⁷⁰)S(O)₂(R⁷⁰), —S(O)R⁷⁰, —S(O)₂R⁷⁰, —S(O)₂N(R⁷⁰)₂, —NO₂, and —CN;
A⁶¹ is represented by *-L⁶¹-C⁶¹ wherein * represents the connection to D;
   L⁶¹ is selected from a bond and -L^{E}-L^{F}-;
      L^{E} is selected from absent, —O—, —S—, and —N(R⁶⁶)—; and
      L^{F} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), —S(O)R⁷¹, —S(O)₂R⁷¹, —S(O)₂N(R⁷¹)₂, ═O, ═S, ═NR⁷¹, —NO₂, and —CN;
   C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —S(O)R⁷², —S(O)₂R⁷², —S(O)₂N(R⁷²)₂, —NO₂, and —CN;
Y² is selected from a bond and C₁₋₄ alkylene optionally substituted with one or more substituents selected from halogen, —OH, —O—C₁₋₄ alkyl, ═O, and ═S;
X³ is CR⁶⁵ or N;
X⁴ is O, C(R⁶⁵)(R⁶¹), or N(R⁶¹);
R⁶¹ is independently selected from:
   hydrogen;
   C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN; and
   5- to 6-membered heterocycle optionally substituted with one or more substituents selected from halogen, C₁₋₄ alkyl, C₁₄ haloalkyl, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN;
R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —OC(O)N(R⁷⁴)₂, —C(O)N(R⁷⁴)₂, —N(R⁷⁴)C(O)R⁷⁴, —N(R⁷⁴)C(O)OR⁷⁴, —N(R⁷⁴)C(O)N(R⁷⁴)₂, —N(R⁷⁴)S(O)₂(R⁷⁴), —S(O)R⁷⁴, —S(O)₂R⁷⁴, —S(O)₂N(R⁷⁴)₂, ═O, ═S, ═NR⁷⁴, —NO₂, and —CN;
R⁶³ and R⁶⁴ are each independently selected at each occurrence from: halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, —N(R⁷⁵)S(O)₂(R⁷⁵), —S(O)R⁷⁵, —S(O)₂R⁷⁵, —S(O)₂N(R⁷⁵)₂, —NO₂, and —CN;
R⁶⁵ is selected from hydrogen, halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, —N(R⁷⁵)S(O)₂(R⁷⁵), —S(O)R⁷⁵, —S(O)₂R⁷⁵, —S(O)₂N(R⁷⁵)₂, —NO₂, and —CN;
R⁶⁶ is selected from hydrogen, C₁₋₆ alkyl, and C₁₋₆ haloalkyl;
R⁷⁰, R⁷¹, R⁷², R⁷³, R⁷⁴, and R⁷⁵ are each independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle;
u is selected from 0, 1, 2, and 3;
v is selected from 0, 1, 2, 3, and 4; and
w and x are each independently selected from 1 and 2.

In some embodiments, the present disclosure provides compounds represented by the structure of Formula (VIIA) or (VIIB):

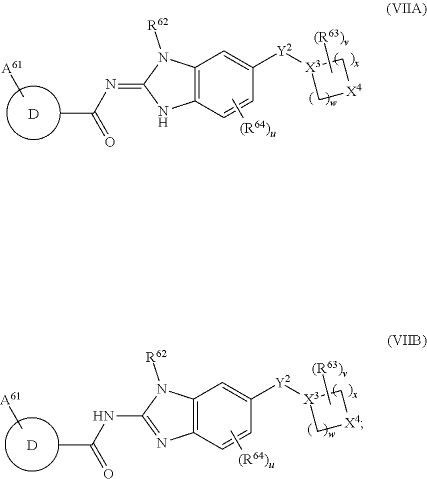

or a pharmaceutically acceptable salt thereof, wherein:
D is a phenyl or a 5- to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —OC(O)N(R⁷⁰)₂, —C(O)N(R⁷⁰)₂, —N(R⁷⁰)C(O)R⁷⁰, —N(R⁷⁰)C(O)OR⁷⁰, —N(R⁷⁰)C(O)N(R⁷⁰)₂, —N(R⁷⁰)S(O)₂(R⁷⁰), —NO₂, and —CN;
A⁶¹ is represented by *-L⁶¹-C⁶¹ wherein * represents the connection to D;
   L⁶¹ is selected from a bond and -L^{E}-L^{F}-;
      L^{E} is selected from absent, —O—, —S—, and —N(R⁶⁶)—; and
      L^{F} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), and ═O;
   C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —NO₂, and —CN;
Y² is selected from a bond and C₁₋₄ alkylene optionally substituted with one or more substituents selected from halogen, —OH, —O—C₁₋₄ alkyl, and ═O;
X³ is CR⁶⁵ or N;
X⁴ is O, C(R⁶⁵)(R⁶¹), or N(R⁶¹);
   R⁶¹ is independently selected from:
hydrogen;
   C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —NO₂, and —CN; and
   5- to 6-membered heterocycle optionally substituted with one or more substituents selected from halogen, C₁₋₄ alkyl, C₁₄ haloalkyl, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —NO₂, and —CN;
R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —OC(O)N(R⁷⁴)₂, —C(O)N(R⁷⁴)₂, —N(R⁷⁴)C(O)R⁷⁴, —N(R⁷⁴)C(O)OR⁷⁴, —N(R⁷⁴)C(O)N(R⁷⁴)₂, —N(R⁷⁴)S(O)₂(R⁷⁴), ═O, ═S, ═NR⁷⁴, —NO₂, and —CN;
R⁶³ and R⁶⁴ are each independently selected at each occurrence from: halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, and —N(R⁷⁵)S(O)₂(R⁷⁵), and —CN;
R⁶⁵ is selected from hydrogen, halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, and —N(R⁷⁵)S(O)₂(R⁷⁵);
R⁶⁶ is selected from hydrogen, C₁₋₆ alkyl, and C₁₋₆ haloalkyl;
R⁷⁰, R⁷¹, R⁷², R⁷³, R⁷⁴, and R⁷⁵ are each independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle;
u is selected from 0, 1, 2, and 3;
v is selected from 0, 1, 2, 3, and 4; and
w and x are each independently selected from 1 and 2.

In some embodiments, the present disclosure provides compounds represented by the structure of Formula (VIIA) or (VIIB):

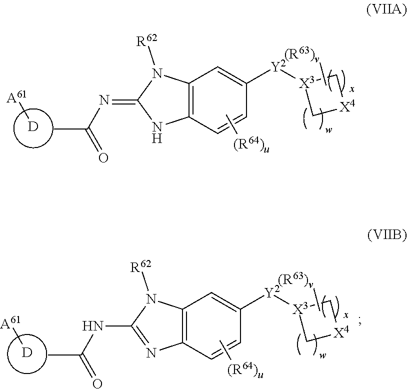

or a pharmaceutically acceptable salt thereof, wherein:
D is a phenyl or a 5- to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —C(O)N(R⁷⁰)₂, —N(R⁷⁰)C(O)R⁷⁰, —N(R⁷⁰)S(O)₂(R⁷⁰), —NO₂, and —CN;
A⁶¹ is represented by *-L⁶¹-C⁶¹ wherein * represents the connection to D;
   L⁶¹ is selected from a bond and -L^{E}-L^{F}-;
      L^{E} is selected from absent, —O—, —S—, and —N(R⁶⁶)—; and
      L^{F} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —C(O)N(R⁷¹)₂, —N(R⁷¹)C(O)R⁷¹, —N(R⁷¹)S(O)₂(R⁷¹), ═O, —NO₂, and —CN;
   C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —NO₂, and —CN;
Y² is selected from a bond and C₁₋₄ alkylene optionally substituted with one or more substituents selected from halogen, —OH, and ═O;
X³ is CR⁶⁵ or N;
X⁴ is O, C(R⁶⁵)(R⁶¹), or N(R⁶¹)
R⁶¹ is independently selected from:
   hydrogen;
   C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, and —N(R⁷³)S(O)₂(R⁷³), —NO₂; and
   5- to 6-membered heterocycle optionally substituted with one or more substituents selected from halogen, C₁₋₄ alkyl, C₁₋₄ haloalkyl, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, —N(R⁷³)S(O)₂(R⁷³), —NO₂, and —CN;
R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —C(O)N(R⁷⁴)₂, —N(R⁷⁴)C(O)R⁷⁴, —N(R⁷⁴)S(O)₂(R⁷⁴), and ═O;
R⁶³ and R⁶⁴ are each independently selected at each occurrence from: halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, and —N(R⁷⁵)S(O)₂(R⁷⁵); and
R⁶⁵ is selected from hydrogen, halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, and —N(R⁷⁵)S(O)₂(R⁷⁵);
R⁶⁶ is selected from hydrogen and C₁₋₆ alkyl;
R⁷⁰, R⁷¹, R⁷², R⁷³, R⁷⁴, and R⁷⁵ are each independently selected at each occurrence from: hydrogen, C₁₋₆ alkyl, C₁₋₆ haloalkyl, C₁₋₆ hydroxyalkyl, and C₃₋₆ carbocycle;
u is selected from 0, 1, 2, and 3;
v is selected from 0, 1, 2, 3, and 4; and
w and x are each independently selected from 1 and 2.

In some embodiments, the present disclosure provides compounds represented by the structure of Formula (VIIA) or (VIIB):

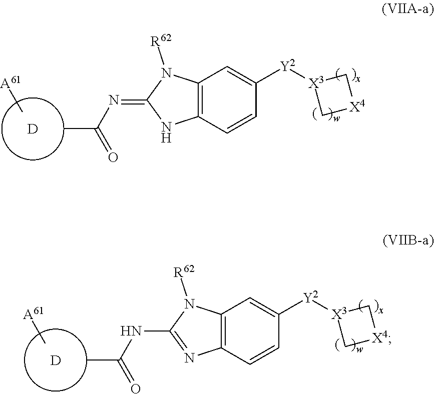

or a pharmaceutically acceptable salt thereof, wherein:
D is a phenyl or a 5- to 10-membered heteroaryl optionally substituted with one or more substituents independently selected from: halogen, C₁₋₆ alkyl, —C(O)R⁷⁰, —N(R⁷⁰)C(O)R⁷⁰, and —CN;
A⁶¹ is represented by *-L⁶¹-C⁶¹ wherein * represents the connection to D;
   L⁶¹ is selected from a bond and -L^{E}-L^{F}-;
      L^{E} is selected from absent, —O—, and —N(R⁶⁶)—; and
      L^{F} is selected from absent, C₁₋₄ alkylene, C₂₋₄ alkynylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —C(O)N(R⁷¹)₂, —N(R⁷¹)C(O)R⁷¹, —N(R⁷¹)S(O)₂(R⁷¹), ═O, —NO₂, and —CN;
   C⁶¹ is selected from phenyl and monocyclic 5- to 6-membered heteroaryl, any of which is substituted with —C(O)H and optionally substituted with one or more substituents independently selected from: halogen, —OR⁷², —N(R⁷²)₂, —N(R⁷²)C(O)R⁷², —N(R⁷²)C(O)OR⁷², and —N(R⁷²)S(O)₂(R⁷²);
Y² is selected from a bond and C₁₋₄ alkylene optionally substituted with one or more ═O;
X³ is N;
X⁴ is O, C(R⁶⁵)(R⁶¹), or N(R⁶¹) R⁶¹ is independently selected from:
   hydrogen;
   C₁₋₆ alkyl optionally substituted with one or more substituents selected from —OR⁷³; and
   5- to 6-membered heterocycle optionally substituted with one or more substituents selected from C₁₋₄ alkyl;
R⁶² is selected from C₁₋₆ alkyl optionally substituted with one or more substituent independently selected at each occurrence from —OR⁷⁴;
R⁶⁵ is selected from hydrogen;
R⁶⁶ is selected from hydrogen and C₁₋₆ alkyl;
R⁷⁰, R⁷¹, R⁷², R⁷³, R⁷⁴, and R⁷⁵ are each independently selected at each occurrence from:
   hydrogen, C₁₋₆ alkyl, C₁₋₆ haloalkyl, and C₃₋₆ carbocycle; and
w and x are each independently selected from 1 and 2.

In some embodiments, D is a phenyl or a 5- to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —OC(O)N(R⁷⁰)₂, —C(O)N(R⁷⁰)₂, —N(R⁷⁰)C(O)R⁷⁰, —N(R⁷⁰)C(O)OR⁷⁰, —N(R⁷⁰)C(O)N(R⁷⁰)₂, —N(R⁷⁰)S(O)₂(R⁷⁰), —S(O)R⁷⁰, —S(O)₂R⁷⁰, —S(O)₂N(R⁷⁰)₂, —NO₂, and —CN. In some embodiments, D is a phenyl or a 5 to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —OC(O)N(R⁷⁰)₂, —C(O)N(R⁷⁰)₂, —N(R⁷⁰)C(O)R⁷⁰, —N(R⁷⁰)C(O)OR⁷⁰, —N(R⁷⁰)C(O)N(R⁷⁰)₂, —N(R⁷⁰)S(O)₂(R⁷⁰), —NO₂, and —CN. In some embodiments, D is a phenyl or a 5- to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —C(O)N(R⁷⁰)₂, —N(R⁷⁰)C(O)R⁷⁰, —N(R⁷⁰)S(O)₂(R⁷⁰), —NO₂, and —CN. In some embodiments, D is a phenyl or a 5- to 10-membered heteroaryl optionally substituted with one or more substituents independently selected from: halogen, C₁₋₆ alkyl, C₁₋₆ haloalkyl, —OR⁷⁰, —N(R⁷⁰)₂, —C(O)R⁷⁰, —C(O)OR⁷⁰, —C(O)N(R⁷⁰)₂, —N(R⁷⁰)C(O)R⁷⁰, and —CN. In some embodiments, D is a phenyl or a 5- to 10-membered heteroaryl optionally substituted with one or more substituents independently selected from: halogen, C₁₋₆ alkyl, —C(O)R⁷⁰, —N(R⁷⁰)C(O)R⁷⁰, and —CN. In some embodiments, D is phenyl, pyridyl, pyrizinyl, thiophenyl, and benzothiophene, each is which is optionally substituted with one or more substituents independently selected from: halogen, C₁₋₆ alkyl, C₁₋₆ haloalkyl, —OR⁷⁰, —N(R⁷⁰)₂, —C(O)R⁷⁰, —C(O)OR⁷⁰, —C(O)N(R⁷⁰)₂, —N(R⁷⁰)C(O)R⁷⁰, and —CN. In some embodiments, D is phenyl, pyridyl, pyrizinyl, thiophenyl, and benzothiophene, each is which is optionally substituted with one or more substituents independently selected from: halogen, C₁₋₆ alkyl, —C(O)R⁷⁰, —N(R⁷⁰)C(O)R⁷⁰, and —CN.

In some embodiments, L⁶¹ is selected from a bond and -L^{E}-L^{F}-. In some embodiments, L⁶¹ is a bond. In some embodiments, L⁶¹ is -L^{E}-L^{F}-.

In some embodiments, L^{E} is selected from absent, —O—, —S—, and —N(R⁶⁶). In some embodiments, L^{E} is selected from absent, —O—, and —N(R⁶⁶). In some embodiments, L^{E} is selected from absent and —O—. In some embodiments, L^{E} is selected from absent and —N(R⁶⁶). In some embodiments, L^{E} is selected from —O— and —N(R⁶⁶). In some embodiments, L^{E} is absent. In some embodiments, L^{E} is —O—. In some embodiments, L^{E} is —S—. In some embodiments, L^{E} is —N(R⁶⁶)

In some embodiments, L^{F} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹ —N(R⁷¹)₂, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), —S(O)R⁷¹, —S(O)₂R⁷¹, —S(O)₂N(R⁷¹)₂, ═O, ═S, ═NR⁷¹, —NO₂, and —CN. In some embodiments, L^{F} is selected from absent, C₁₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), ═O, —NO₂, and —CN. In some embodiments, L^{F} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —C(O)N(R⁷¹)₂, —N(R⁷¹)C(O)R⁷¹, —N(R⁷¹)S(O)₂(R⁷¹), ═O, —NO₂, and —CN. In some embodiments, L^{F} is selected from absent, C₁₄ alkylene, C₂₋₄ alkynylene, phenylene, and 4- to 6-membered heterocyclene, each of which is optionally substituted with one or more substituents independently selected from: halogen, —OR⁷², —SR⁷², —N(R⁷²)₂, —C(O)R⁷², —C(O)OR⁷², —OC(O)R⁷², —C(O)N(R⁷²)₂, —N(R⁷²)C(O)R⁷², ═O, and —CN. In some embodiments, L^{F} is selected from absent, C₁₋₄ alkylene, C₂₋₄ alkynylene, and 4- to 6-membered heterocyclene, each of which is optionally substituted with one or more substituents independently selected from: halogen, —OR⁷², —SR⁷², —N(R⁷²)₂, and ═O. In some embodiments, L^{F} is selected from absent, C₁₋₄ alkylene, C₂₋₄ alkynylene, and 4- to 6-membered heterocyclene. In some embodiments, L^{F} is C₁₋₄ alkylene. In some embodiments, L^{F} is C₂₋₄ alkynylene. In some embodiments, L^{F} is 4- to 6-membered heterocyclene. In some embodiments, L^{F} is selected from absent, methylene, ethylene, azetidinylene, and pyrazolylene. In some embodiments, L^{F} is absent. In some embodiments, L^{F} is methylene. In some embodiments, L^{F} is ethylene. In some embodiments, L^{F} is azetidinylene. In some embodiments, L^{F} is pyrazolylene.

In some embodiments, L⁶¹ is selected from a bond, —O—, —OCH₂—, —N(CH₃)—, —N(CH₃)CH₂—, ethynylene,

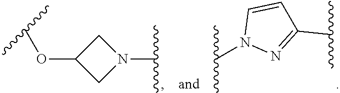 [SMILES: *OC1CN(*)C1 ; *c1ccn(*)n1]

In some embodiments, C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —S(O)R⁷², —S(O)₂R⁷², —S(O)₂N(R⁷²)₂, —NO₂, and —CN. In some embodiments, C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —NO₂, and —CN. In some embodiments, C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —NO₂, and —CN. In some embodiments, C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —NO₂, and —CN. In some embodiments, C⁶¹ is selected from phenyl and monocyclic 5- to 6-membered heteroaryl, any of which is substituted with —C(O)H and optionally substituted with one or more substituents independently selected from: halogen, —OR⁷², —N(R⁷²)₂, —N(R⁷²)C(O)R⁷², —N(R⁷²)C(O)OR⁷², and —N(R⁷²)S(O)₂(R⁷²). In some embodiments, C⁶¹ is selected from phenyl and monocyclic 5- to 6-membered heteroaryl, any of which is substituted with —C(O)H and —OH, and optionally substituted with one or more substituents independently selected from halogen. In some embodiments, C⁶¹ is selected from:

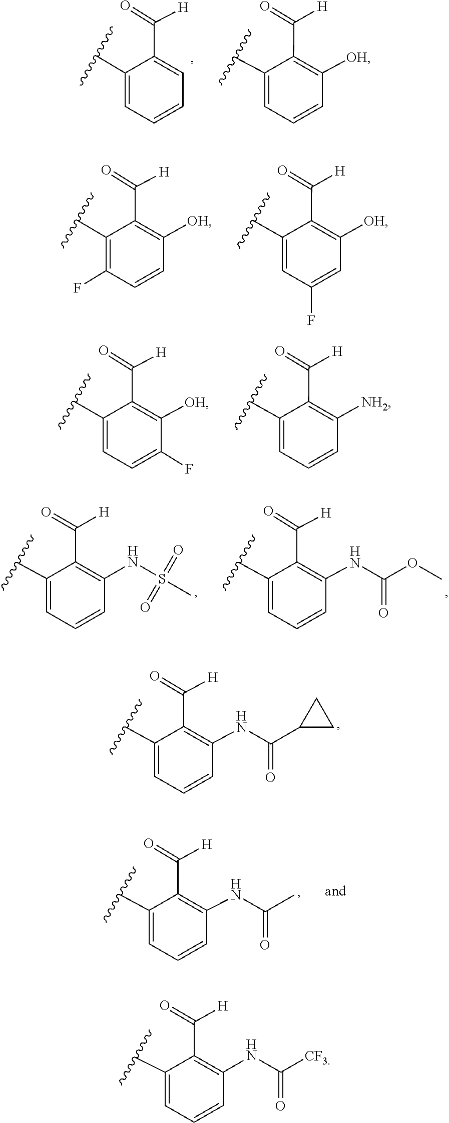

In some embodiments, Y² is selected from a bond and C₁₋₄ alkylene optionally substituted with one or more substituents selected from halogen, —OH, —O—C₁₋₄ alkyl, ═O, and ═S. In some embodiments, Y² is selected from a bond and C₁₋₄ alkylene optionally substituted with one or more substituents selected from halogen, —OH, —O—C₁₋₄ alkyl, and ═O. In some embodiments, Y² is selected from a bond and C₁₋₄ alkylene optionally substituted with one or more substituents selected from halogen, —OH, and ═O. In some embodiments, Y² is selected from a bond and C₁₋₄ alkylene optionally substituted with one or more ═O. In some embodiments, Y² is selected from a bond, methylene, and —C(═O)—. In some embodiments, Y² is a bond. In some embodiments, Y² is methylene. In some embodiments, Y² is —C(═O)—.

In some embodiments, X³ is CR⁶⁵ or N. In some embodiments, X³ is CR⁶⁵. In some embodiments, X³ is N.

In some embodiments, X⁴ is O, C(R⁶⁵)(R⁶¹), or N(R⁶¹). In some embodiments, X⁴ is O or C(R⁶⁵)(R⁶¹). In some embodiments, X⁴ is O or N(R⁶¹). In some embodiments, X⁴ is C(R⁶⁵)(R⁶¹) or N(R⁶¹). In some embodiments, X⁴ is 0. In some embodiments, X⁴ is C(R⁶⁵)(R⁶¹).

In some embodiments, X⁴ is N(R⁶¹)

In some embodiments, R⁶¹ is independently selected from:
hydrogen;
C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN; and 5- to 6-membered heterocycle optionally substituted with one or more substituents selected from halogen, C₁₋₄ alkyl, C₁₋₄ haloalkyl, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN.

In some embodiments, R⁶¹ is independently selected from:
hydrogen;
C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —NO₂, and —CN; and
5- to 6-membered heterocycle optionally substituted with one or more substituents selected from halogen, C₁₋₄ alkyl, C₁₋₄ haloalkyl, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —NO₂, and —CN.

In some embodiments, R⁶¹ is independently selected from:
hydrogen;
C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, and —N(R⁷³)S(O)₂(R⁷³), —NO₂; and
5- to 6-membered heterocycle optionally substituted with one or more substituents selected from halogen, C₁₋₄ alkyl, C₁₋₄ haloalkyl, —OR⁷³, —SR⁷³, —N(R⁷³)₂, —C(O)R⁷³, —C(O)OR⁷³, —OC(O)R⁷³, —C(O)N(R⁷³)₂, —N(R⁷³)C(O)R⁷³, —N(R⁷³)S(O)₂(R⁷³), —NO₂, and —CN.

In some embodiments, R⁶¹ is independently selected from:
hydrogen;
C₁₋₆ alkyl optionally substituted with one or more substituents selected from —OR⁷³; and
5- to 6-membered heterocycle optionally substituted with one or more substituents selected from C₁₋₄ alkyl.

In some embodiments, R⁶¹ is independently selected from: hydrogen; C₁₋₆ alkyl optionally substituted with one or more substituents selected from halogen and —OR⁷³; and 5- to 6-membered heterocycle optionally substituted with one or more substituents selected from halogen, C₁₋₄ alkyl, C₁₄ haloalkyl, and —OR⁷³. In some embodiments, R⁶¹ is independently selected from: methyl,

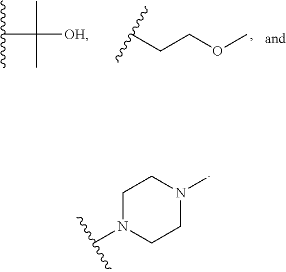

In some embodiments, R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —OC(O)N(R⁷⁴)₂, —C(O)N(R⁷⁴)₂, —N(R⁷⁴)C(O)R⁷⁴, —N(R⁷⁴)C(O)OR⁷⁴, —N(R⁷⁴)C(O)N(R⁷⁴)₂, —N(R⁷⁴)S(O)₂(R⁷⁴), —S(O)R⁷⁴, —S(O)₂R⁷⁴, —S(O)₂N(R⁷⁴)₂, ═O, ═S, ═NR⁷⁴, —NO₂, and —CN. In some embodiments, R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —OC(O)N(R⁷⁴)₂, —C(O)N(R⁷⁴)₂, —N(R⁷⁴)C(O)R⁷⁴, —N(R⁷⁴)C(O)OR⁷⁴, —N(R⁷⁴)C(O)N(R⁷⁴)₂, —N(R⁷⁴)S(O)₂(R⁷⁴), ═O, ═S, ═NR⁷⁴, —NO₂, and —CN. In some embodiments, R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —C(O)N(R⁷⁴)₂, —N(R⁷⁴)C(O)R⁷⁴, —N(R⁷⁴)S(O)₂(R⁷⁴), and ═O. In some embodiments, R⁶² is selected from C₁₋₆ alkyl optionally substituted with one or more substituent independently selected at each occurrence from: halogen, —OR⁷⁴, and —CN. In some embodiments, R⁶² is selected from C₁₋₆ alkyl optionally substituted with one or more substituent independently selected at each occurrence from —OR⁷⁴. In some embodiments, R⁶² is selected from:

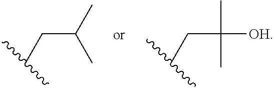

In some embodiments, w and x are each independently selected from 1 and 2. In some embodiments, w and x are each 1. In some embodiments, w and x are each 2. In some embodiments, w is selected from 1 and 2. In some embodiments, w is 1. In some embodiments, w is 2. In some embodiments, x is selected from 1 and 2. In some embodiments, x is 1. In some embodiments, x is 2.

In some embodiments, v is selected from 0, 1, 2, 3, and 4. In some embodiments, v is selected from 0, 1, 2, and 3. In some embodiments, v is selected from 0, 1, and 2. In some embodiments, v is selected from 0 and 1. In some embodiments, v is selected from 0. In some embodiments, v is selected from 1. In some embodiments, v is selected from 2. In some embodiments, v is selected from 3. In some embodiments, v is selected from 4.

In some embodiments, u is selected from 0, 1, 2, and 3. In some embodiments, u is selected from 0, 1, and 2. In some embodiments, u is selected from 0 and 1. In some embodiments, u is selected from 0. In some embodiments, u is selected from 1. In some embodiments, u is selected from 2. In some embodiments, u is selected from 3.

In some embodiments, R⁷⁰, R⁷¹, R⁷², R⁷³, R⁷⁴, and R⁷⁵ are each independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle. In some embodiments, R⁷⁰, R⁷¹, R⁷², R⁷³, R⁷⁴, and R⁷⁵ are each independently selected at each occurrence from: hydrogen, C₁₋₆ alkyl, C₁₋₆ haloalkyl, C₁₋₆ hydroxyalkyl, and C₃₋₆ carbocycle. In some embodiments, R⁷⁰, R⁷¹, R⁷², R⁷³, R⁷⁴, and R⁷⁵ are each independently selected at each occurrence from: hydrogen, C₁₋₆ alkyl, C₁₋₆ haloalkyl, and C₃₋₆ carbocycle. In some embodiments, R⁷⁰, R⁷¹, R⁷², R⁷³, R⁷⁴, and R⁷⁵ are each hydrogen. In some embodiments, R⁷⁰, R⁷¹, R⁷², R⁷³, R⁷⁴, and R⁷⁵ are each C₁₋₆ alkyl. In some embodiments, R⁷⁰, R⁷¹, R⁷², R⁷³, R⁷⁴, and R⁷⁵ are each C₁₋₆ haloalkyl. In some embodiments, R⁷⁰, R⁷¹, R⁷², R⁷³, R⁷⁴, and R⁷⁵ are each C₃₋₆ carbocycle.

In some embodiments, R⁷⁰ is independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle. In some embodiments, R⁷⁰ is independently selected at each occurrence from: hydrogen, C₁₋₆ alkyl, C₁₋₆ haloalkyl, C₁₋₆ hydroxyalkyl, and C₃₋₆ carbocycle. In some embodiments, R⁷⁰ is independently selected at each occurrence from: hydrogen, C₁₋₆ alkyl, C₁₋₆ haloalkyl, and C₃₋₆ carbocycle. In some embodiments, each R⁷⁰ is hydrogen. In some embodiments, each R⁷⁰ is C₁₋₆ alkyl. In some embodiments, each R⁷⁰ is C₁₋₆ haloalkyl. In some embodiments, each R⁷⁰ is C₃₋₆ carbocycle.

In some embodiments, R⁷¹ is independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle.

In some embodiments, R⁷¹ is independently selected at each occurrence from: hydrogen, C₁₋₆ alkyl, C₁₋₆ haloalkyl, C₁₋₆ hydroxyalkyl, and C₃₋₆ carbocycle. In some embodiments, R⁷¹ is independently selected at each occurrence from: hydrogen, C₁₋₆ alkyl, C₁₋₆ haloalkyl, and C₃₋₆ carbocycle. In some embodiments, each R⁷¹ is hydrogen. In some embodiments, each R⁷¹ is C₁₋₆ alkyl. In some embodiments, each R⁷¹ is C₁₋₆ haloalkyl. In some embodiments, each R⁷¹ is C₃₋₆ carbocycle.

In some embodiments, R⁷² is independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle. In some embodiments, R⁷² is independently selected at each occurrence from: hydrogen, C₁₋₆ alkyl, C₁₋₆ haloalkyl, C₁₋₆ hydroxyalkyl, and C₃₋₆ carbocycle. In some embodiments, R⁷² is independently selected at each occurrence from: hydrogen, C₁₋₆ alkyl, C₁₋₆ haloalkyl, and C₃₋₆ carbocycle. In some embodiments, each R⁷² is hydrogen. In some embodiments, each R⁷² is C₁₋₆ alkyl. In some embodiments, each R⁷² is C₁₋₆ haloalkyl. In some embodiments, each R⁷² is C₃₋₆ carbocycle.

In some embodiments, R⁷³ is independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle. In some embodiments, R⁷³ is independently selected at each occurrence from: hydrogen, C₁₋₆ alkyl, C₁₋₆ haloalkyl, C₁₋₆ hydroxyalkyl, and C₃₋₆ carbocycle. In some embodiments, R⁷³ is independently selected at each occurrence from: hydrogen, C₁₋₆ alkyl, C₁₋₆ haloalkyl, and C₃₋₆ carbocycle. In some embodiments, each R⁷³ is hydrogen. In some embodiments, each R⁷³ is C₁₋₆ alkyl. In some embodiments, each R⁷³ is C₁₋₆ haloalkyl. In some embodiments, each R⁷³ is C₃₋₆ carbocycle.

In some embodiments, R⁷⁴ is independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle. In some embodiments, R⁷⁴ is independently selected at each occurrence from: hydrogen, C₁₋₆ alkyl, C₁₋₆ haloalkyl, C₁₋₆ hydroxyalkyl, and C₃₋₆ carbocycle. In some embodiments, R⁷⁴ is independently selected at each occurrence from: hydrogen, C₁₋₆ alkyl, C₁₋₆ haloalkyl, and C₃₋₆ carbocycle. In some embodiments, each R⁷⁴ is hydrogen. In some embodiments, each R⁷⁴ is C₁₋₆ alkyl. In some embodiments, each R⁷⁴ is C₁₋₆ haloalkyl. In some embodiments, each R⁷⁴ is C₃₋₆ carbocycle.

In some embodiments, R⁷⁵ is independently selected at each occurrence from: hydrogen, C₁₋₆ alkyl, C₁₋₆ haloalkyl, C₁₋₆ hydroxyalkyl, C₃₋₆ carbocycle, and 3- to 6-membered heterocycle. In some embodiments, R⁷⁵ is independently selected at each occurrence from: hydrogen, C₁₋₆ alkyl, C₁₋₆ haloalkyl, C₁₋₆ hydroxyalkyl, and C₃₋₆ carbocycle. In some embodiments, R⁷⁵ is independently selected at each occurrence from: hydrogen, C₁₋₆ alkyl, C₁₋₆ haloalkyl, and C₃₋₆ carbocycle. In some embodiments, each R⁷⁵ is hydrogen. In some embodiments, each R⁷⁵ is C₁₋₆ alkyl. In some embodiments, each R⁷⁵ is C₁₋₆ haloalkyl. In some embodiments, each R⁷⁵ is C₃₋₆ carbocycle.

In some embodiments, the compound is selected from a compound of Table 3, or a pharmaceutically acceptable salt thereof.

TABLE 3

| Cmpd. No. | Structure |
|---|---|
| 51 | 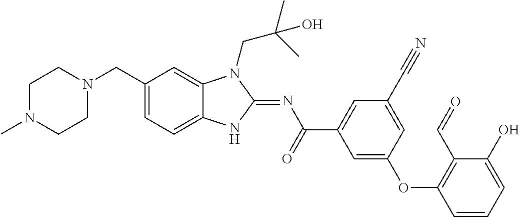 [SMILES: CN1CCN(Cc2ccc3[nH]/c(=N\C(=O)c4cc(C#N)cc(Oc5cccc(O)c5C=O)c4)n(CC(C)(C)O)c3c2)CC1] |
| 52 | 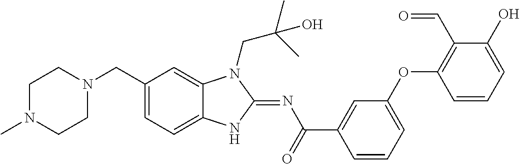 [SMILES: CN1CCN(Cc2ccc3[nH]/c(=N\C(=O)c4cccc(Oc5cccc(O)c5C=O)c4)n(CC(C)(C)O)c3c2)CC1] |
| 53 | 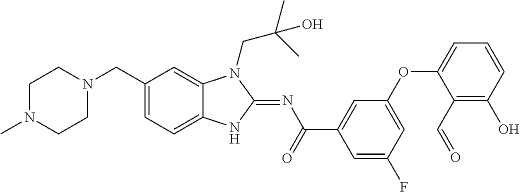 [SMILES: CN1CCN(Cc2ccc3[nH]/c(=N\C(=O)c4cc(F)cc(Oc5cccc(O)c5C=O)c4)n(CC(C)(C)O)c3c2)CC1] |
| 54 | 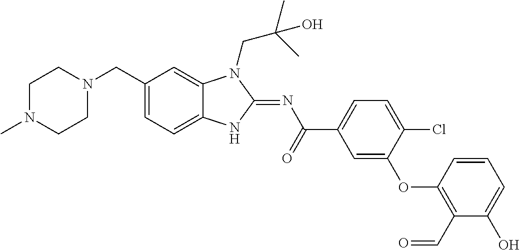 [SMILES: CN1CCN(Cc2ccc3[nH]/c(=N\C(=O)c4ccc(Cl)c(Oc5cccc(O)c5C=O)c4)n(CC(C)(C)O)c3c2)CC1] |
| 55 | 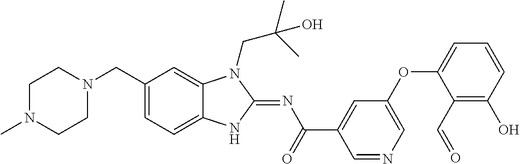 [SMILES: CN1CCN(Cc2ccc3[nH]/c(=N\C(=O)c4cncc(Oc5cccc(O)c5C=O)c4)n(CC(C)(C)O)c3c2)CC1] |
| 56 | 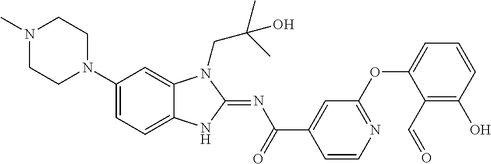 [SMILES: CN1CCN(c2ccc3[nH]/c(=N\C(=O)c4ccnc(Oc5cccc(O)c5C=O)c4)n(CC(C)(C)O)c3c2)CC1] |
| 57 | 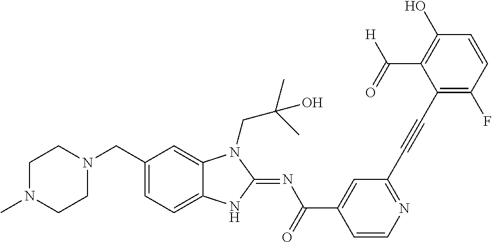 [SMILES: CN1CCN(Cc2ccc3[nH]/c(=N\C(=O)c4ccnc(C#Cc5c(F)ccc(O)c5C=O)c4)n(CC(C)(C)O)c3c2)CC1] |
| 58 | 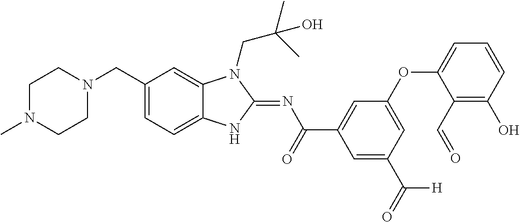 [SMILES: CN1CCN(Cc2ccc3[nH]/c(=N\C(=O)c4cc(C=O)cc(Oc5cccc(O)c5C=O)c4)n(CC(C)(C)O)c3c2)CC1] |
| 59 | 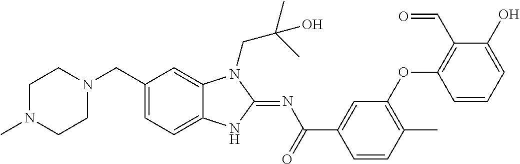 [SMILES: Cc1ccc(C(=O)/N=c2\[nH]c3ccc(CN4CCN(C)CC4)cc3n2CC(C)(C)O)cc1Oc1cccc(O)c1C=O] |
| 60 | 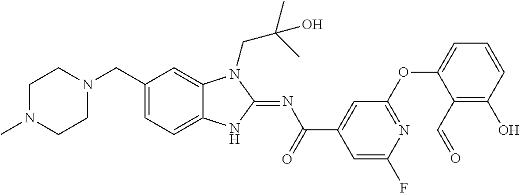 [SMILES: CN1CCN(Cc2ccc3[nH]/c(=N\C(=O)c4cc(F)nc(Oc5cccc(O)c5C=O)c4)n(CC(C)(C)O)c3c2)CC1] |
| 61 | 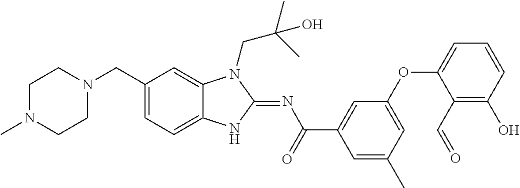 [SMILES: Cc1cc(Oc2cccc(O)c2C=O)cc(C(=O)/N=c2\[nH]c3ccc(CN4CCN(C)CC4)cc3n2CC(C)(C)O)c1] |
| 62 | 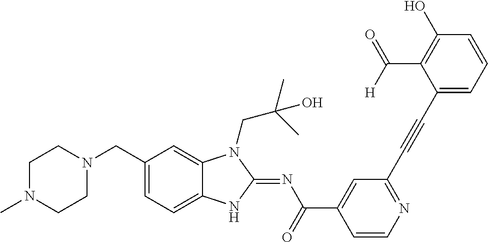 [SMILES: CN1CCN(Cc2ccc3[nH]/c(=N\C(=O)c4ccnc(C#Cc5cccc(O)c5C=O)c4)n(CC(C)(C)O)c3c2)CC1] |
| 63 | 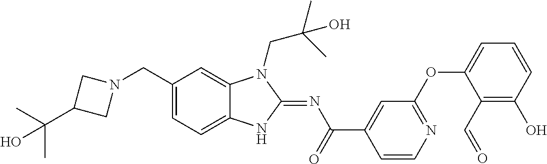 [SMILES: CC(C)(O)Cn1/c(=N/C(=O)c2ccnc(Oc3cccc(O)c3C=O)c2)[nH]c2ccc(CN3CC(C(C)(C)O)C3)cc21] |
| 64 | 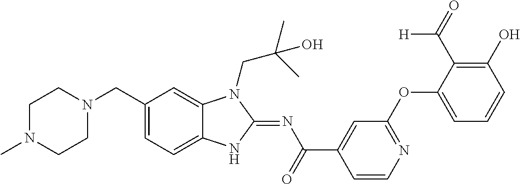 [SMILES: CN1CCN(Cc2ccc3[nH]/c(=N\C(=O)c4ccnc(Oc5cccc(O)c5C=O)c4)n(CC(C)(C)O)c3c2)CC1] |
| 65 | 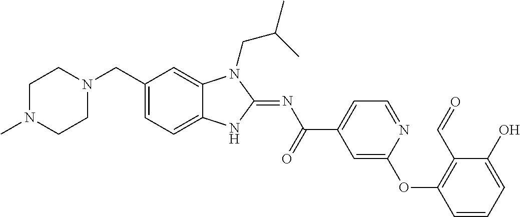 [SMILES: CC(C)Cn1/c(=N/C(=O)c2ccnc(Oc3cccc(O)c3C=O)c2)[nH]c2ccc(CN3CCN(C)CC3)cc21] |
| 66 | 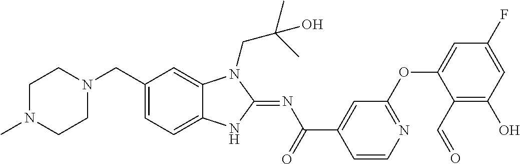 [SMILES: CN1CCN(Cc2ccc3[nH]/c(=N\C(=O)c4ccnc(Oc5cc(F)cc(O)c5C=O)c4)n(CC(C)(C)O)c3c2)CC1] |
| 67 | 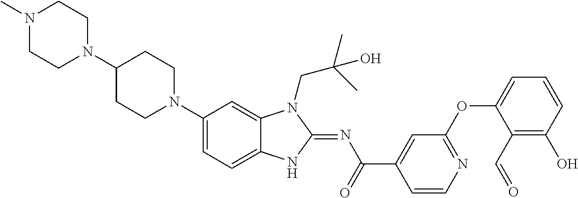 [SMILES: CN1CCN(C2CCN(c3ccc4[nH]/c(=N\C(=O)c5ccnc(Oc6cccc(O)c6C=O)c5)n(CC(C)(C)O)c4c3)CC2)CC1] |
| 68 | 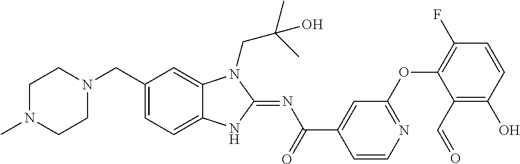 [SMILES: CN1CCN(Cc2ccc3[nH]/c(=N\C(=O)c4ccnc(Oc5c(F)ccc(O)c5C=O)c4)n(CC(C)(C)O)c3c2)CC1] |
| 69 | 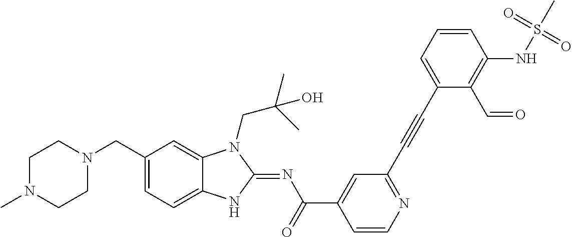 [SMILES: CN1CCN(Cc2ccc3[nH]/c(=N\C(=O)c4ccnc(C#Cc5cccc(NS(C)(=O)=O)c5C=O)c4)n(CC(C)(C)O)c3c2)CC1] |
| 70 | 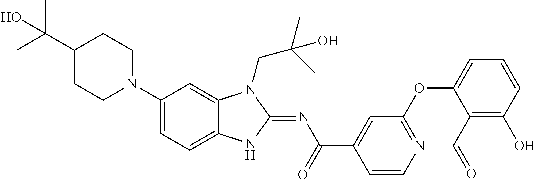 [SMILES: CC(C)(O)Cn1/c(=N/C(=O)c2ccnc(Oc3cccc(O)c3C=O)c2)[nH]c2ccc(N3CCC(C(C)(C)O)CC3)cc21] |
| 71 | 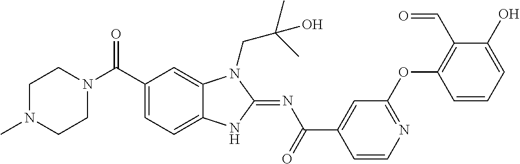 [SMILES: CN1CCN(C(=O)c2ccc3[nH]/c(=N\C(=O)c4ccnc(Oc5cccc(O)c5C=O)c4)n(CC(C)(C)O)c3c2)CC1] |
| 72 | 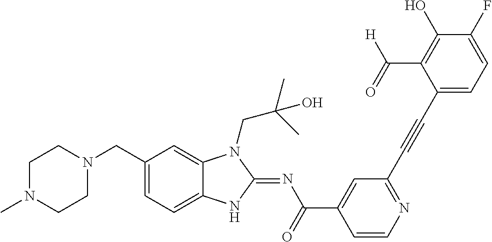 [SMILES: CN1CCN(Cc2ccc3[nH]/c(=N\C(=O)c4ccnc(C#Cc5ccc(F)c(O)c5C=O)c4)n(CC(C)(C)O)c3c2)CC1] |
| 73 | 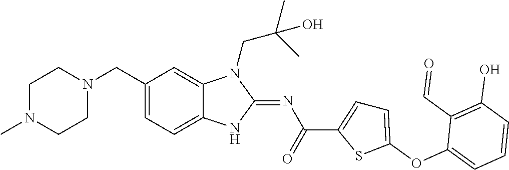 [SMILES: CN1CCN(Cc2ccc3[nH]/c(=N\C(=O)c4ccc(Oc5cccc(O)c5C=O)s4)n(CC(C)(C)O)c3c2)CC1] |
| 74 | 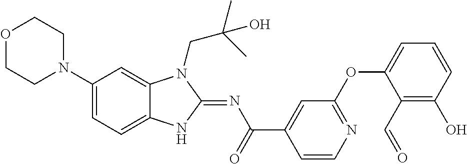 [SMILES: CC(C)(O)Cn1/c(=N/C(=O)c2ccnc(Oc3cccc(O)c3C=O)c2)[nH]c2ccc(N3CCOCC3)cc21] |
| 75 | 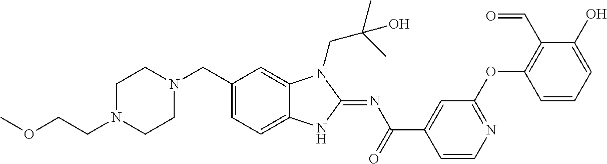 [SMILES: COCCN1CCN(Cc2ccc3[nH]/c(=N\C(=O)c4ccnc(Oc5cccc(O)c5C=O)c4)n(CC(C)(C)O)c3c2)CC1] |
| 76 | 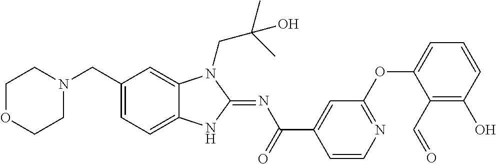 [SMILES: CC(C)(O)Cn1/c(=N/C(=O)c2ccnc(Oc3cccc(O)c3C=O)c2)[nH]c2ccc(CN3CCOCC3)cc21] |
| 77 | 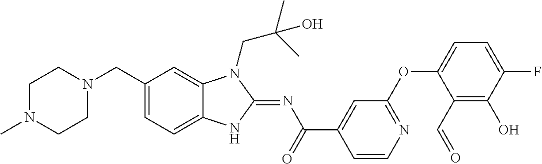 [SMILES: CN1CCN(Cc2ccc3[nH]/c(=N\C(=O)c4ccnc(Oc5ccc(F)c(O)c5C=O)c4)n(CC(C)(C)O)c3c2)CC1] |
| 78 | 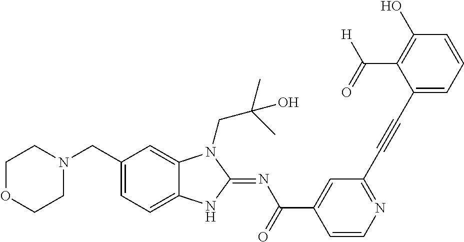 [SMILES: CC(C)(O)Cn1/c(=N/C(=O)c2ccnc(C#Cc3cccc(O)c3C=O)c2)[nH]c2ccc(CN3CCOCC3)cc21] |
| 79 | 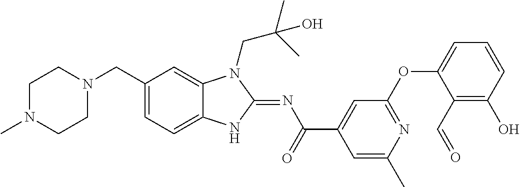 [SMILES: Cc1cc(C(=O)/N=c2\[nH]c3ccc(CN4CCN(C)CC4)cc3n2CC(C)(C)O)cc(Oc2cccc(O)c2C=O)n1] |
| 80 | 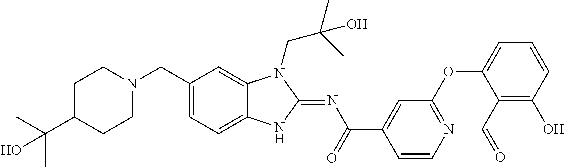 [SMILES: CC(C)(O)Cn1/c(=N/C(=O)c2ccnc(Oc3cccc(O)c3C=O)c2)[nH]c2ccc(CN3CCC(C(C)(C)O)CC3)cc21] |
| 81 | 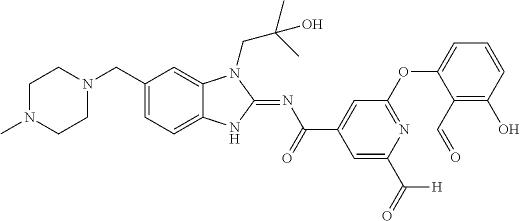 [SMILES: CN1CCN(Cc2ccc3[nH]/c(=N\C(=O)c4cc(C=O)nc(Oc5cccc(O)c5C=O)c4)n(CC(C)(C)O)c3c2)CC1] |
| 82 | 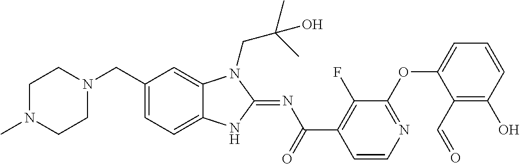 [SMILES: CN1CCN(Cc2ccc3[nH]/c(=N\C(=O)c4ccnc(Oc5cccc(O)c5C=O)c4F)n(CC(C)(C)O)c3c2)CC1] |
| 83 | 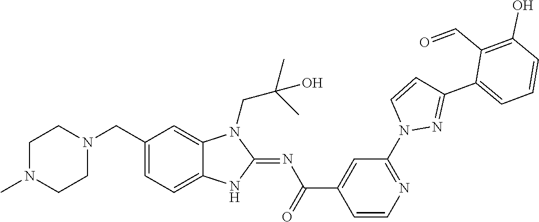 [SMILES: CN1CCN(Cc2ccc3[nH]/c(=N\C(=O)c4ccnc(-n5ccc(-c6cccc(O)c6C=O)n5)c4)n(CC(C)(C)O)c3c2)CC1] |
| 84 | 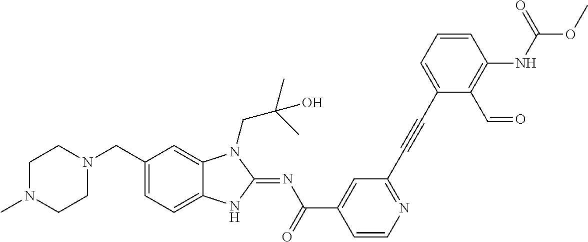 |
| 85 | 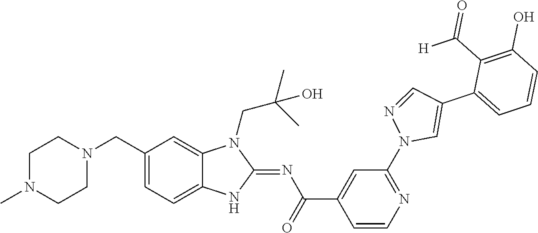 [SMILES: CN1CCN(Cc2ccc3[nH]/c(=N\C(=O)c4ccnc(-n5cc(-c6cccc(O)c6C=O)cn5)c4)n(CC(C)(C)O)c3c2)CC1] |
| 86 | 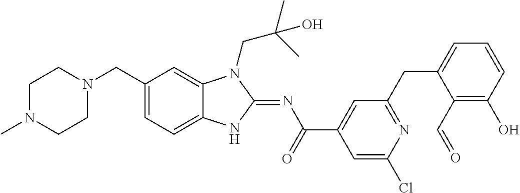 [SMILES: CN1CCN(Cc2ccc3[nH]/c(=N\C(=O)c4cc(Cl)nc(Cc5cccc(O)c5C=O)c4)n(CC(C)(C)O)c3c2)CC1] |
| 87 | 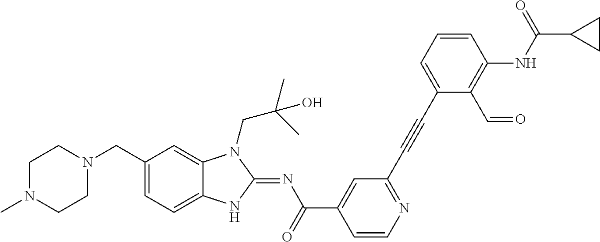 [SMILES: CN1CCN(Cc2ccc3[nH]/c(=N\C(=O)c4ccnc(C#Cc5cccc(NC(=O)C6CC6)c5C=O)c4)n(CC(C)(C)O)c3c2)CC1] |
| 88 | 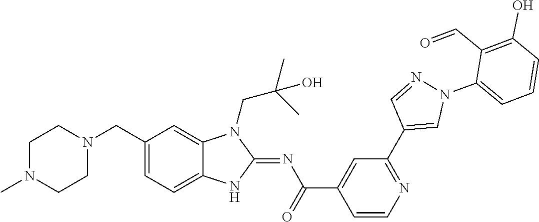 [SMILES: CN1CCN(Cc2ccc3[nH]/c(=N\C(=O)c4ccnc(-c5cnn(-c6cccc(O)c6C=O)c5)c4)n(CC(C)(C)O)c3c2)CC1] |
| 89 | 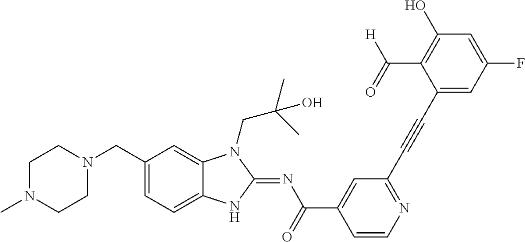 [SMILES: CN1CCN(Cc2ccc3[nH]/c(=N\C(=O)c4ccnc(C#Cc5cc(F)cc(O)c5C=O)c4)n(CC(C)(C)O)c3c2)CC1] |
| 90 | 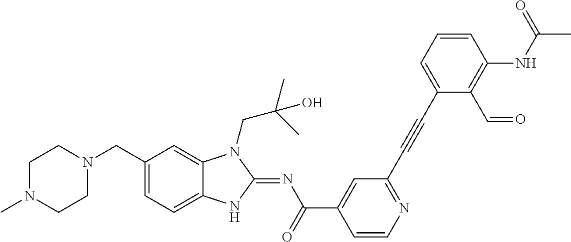 [SMILES: CC(=O)Nc1cccc(C#Cc2cc(C(=O)/N=c3\[nH]c4ccc(CN5CCN(C)CC5)cc4n3CC(C)(C)O)ccn2)c1C=O] |
| 91 | 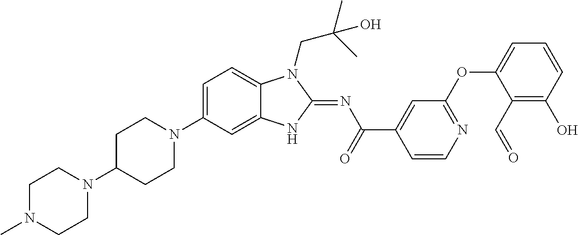 [SMILES: CN1CCN(C2CCN(c3ccc4c(c3)[nH]/c(=N\C(=O)c3ccnc(Oc5cccc(O)c5C=O)c3)n4CC(C)(C)O)CC2)CC1] |
| 92 | 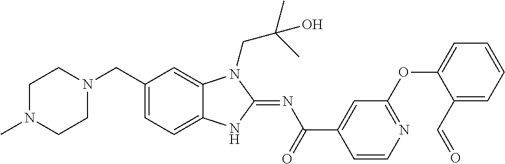 [SMILES: CN1CCN(Cc2ccc3[nH]/c(=N\C(=O)c4ccnc(Oc5ccccc5C=O)c4)n(CC(C)(C)O)c3c2)CC1] |
| 93 | 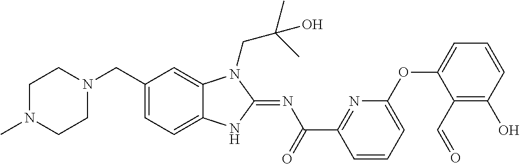 [SMILES: CN1CCN(Cc2ccc3[nH]/c(=N\C(=O)c4cccc(Oc5cccc(O)c5C=O)n4)n(CC(C)(C)O)c3c2)CC1] |
| 94 | 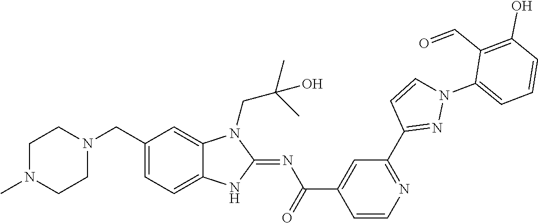 [SMILES: CN1CCN(Cc2ccc3[nH]/c(=N\C(=O)c4ccnc(-c5ccn(-c6cccc(O)c6C=O)n5)c4)n(CC(C)(C)O)c3c2)CC1] |
| 95 | 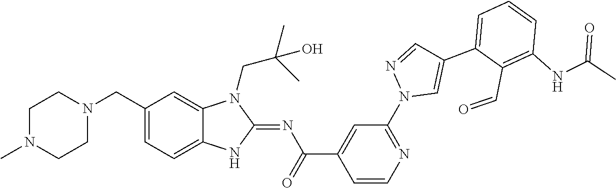 [SMILES: CC(=O)Nc1cccc(-c2cnn(-c3cc(C(=O)/N=c4\[nH]c5ccc(CN6CCN(C)CC6)cc5n4CC(C)(C)O)ccn3)c2)c1C=O] |
| 96 | 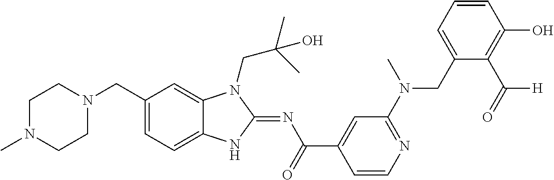 [SMILES: CN1CCN(Cc2ccc3[nH]/c(=N\C(=O)c4ccnc(N(C)Cc5cccc(O)c5C=O)c4)n(CC(C)(C)O)c3c2)CC1] |
| 97 | 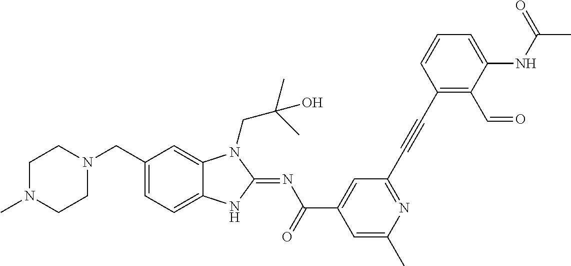 [SMILES: CC(=O)Nc1cccc(C#Cc2cc(C(=O)/N=c3\[nH]c4ccc(CN5CCN(C)CC5)cc4n3CC(C)(C)O)cc(C)n2)c1C=O] |
| 98 | 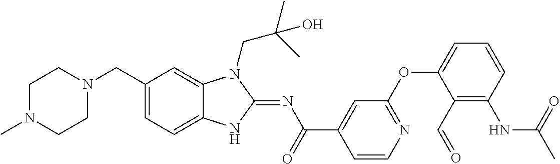 [SMILES: CC(=O)Nc1cccc(Oc2cc(C(=O)/N=c3\[nH]c4ccc(CN5CCN(C)CC5)cc4n3CC(C)(C)O)ccn2)c1C=O] |
| 99 | 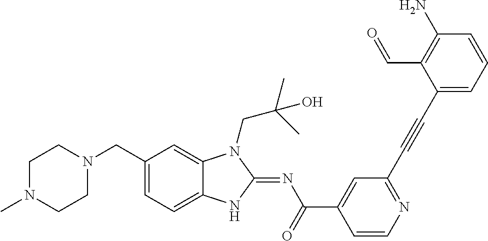 [SMILES: CN1CCN(Cc2ccc3[nH]/c(=N\C(=O)c4ccnc(C#Cc5cccc(N)c5C=O)c4)n(CC(C)(C)O)c3c2)CC1] |
| 100 | 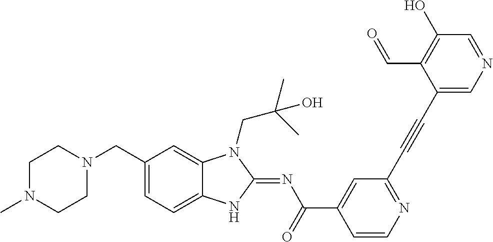 [SMILES: CN1CCN(Cc2ccc3[nH]/c(=N\C(=O)c4ccnc(C#Cc5cncc(O)c5C=O)c4)n(CC(C)(C)O)c3c2)CC1] |
| 101 | 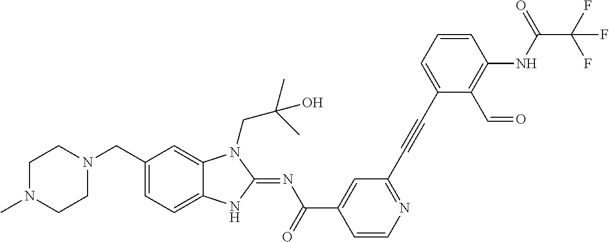 [SMILES: CN1CCN(Cc2ccc3[nH]/c(=N\C(=O)c4ccnc(C#Cc5cccc(NC(=O)C(F)(F)F)c5C=O)c4)n(CC(C)(C)O)c3c2)CC1] |
| 102 | 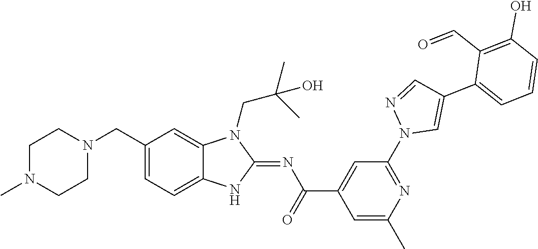 [SMILES: Cc1cc(C(=O)/N=c2\[nH]c3ccc(CN4CCN(C)CC4)cc3n2CC(C)(C)O)cc(-n2cc(-c3cccc(O)c3C=O)cn2)n1] |
| 103 | 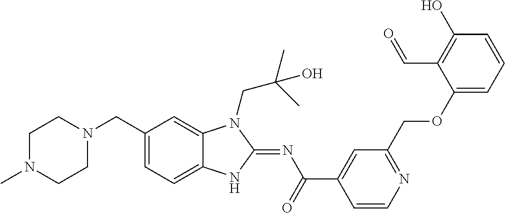 [SMILES: CN1CCN(Cc2ccc3[nH]/c(=N\C(=O)c4ccnc(COc5cccc(O)c5C=O)c4)n(CC(C)(C)O)c3c2)CC1] |
| 104 | 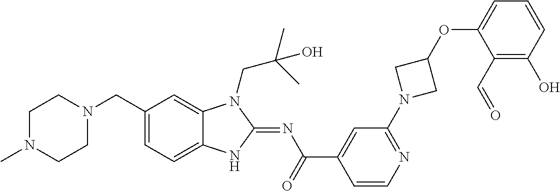 [SMILES: CN1CCN(Cc2ccc3[nH]/c(=N\C(=O)c4ccnc(N5CC(Oc6cccc(O)c6C=O)C5)c4)n(CC(C)(C)O)c3c2)CC1] |
| 105 | 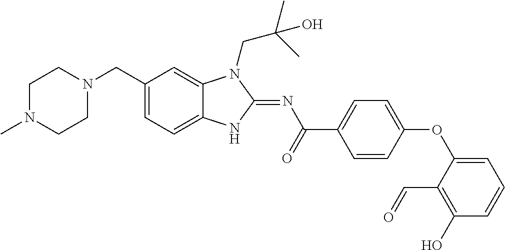 [SMILES: CN1CCN(Cc2ccc3[nH]/c(=N\C(=O)c4ccc(Oc5cccc(O)c5C=O)cc4)n(CC(C)(C)O)c3c2)CC1] |
| 106 | 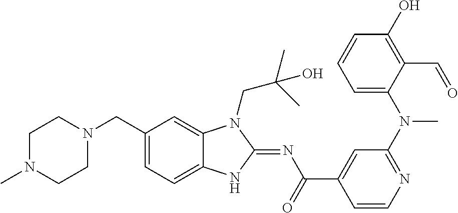 [SMILES: CN1CCN(Cc2ccc3[nH]/c(=N\C(=O)c4ccnc(N(C)c5cccc(O)c5C=O)c4)n(CC(C)(C)O)c3c2)CC1] |
| 107 | 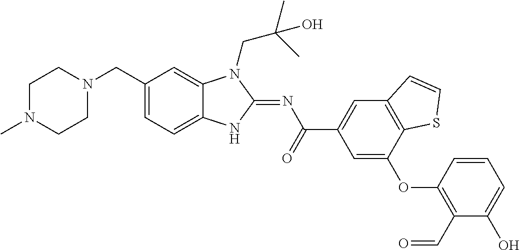 [SMILES: CN1CCN(Cc2ccc3[nH]/c(=N\C(=O)c4cc(Oc5cccc(O)c5C=O)c5sccc5c4)n(CC(C)(C)O)c3c2)CC1] |
| 108 | 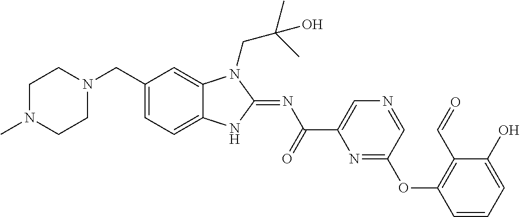 [SMILES: CN1CCN(Cc2ccc3[nH]/c(=N\C(=O)c4cncc(Oc5cccc(O)c5C=O)n4)n(CC(C)(C)O)c3c2)CC1] |
| 109 | 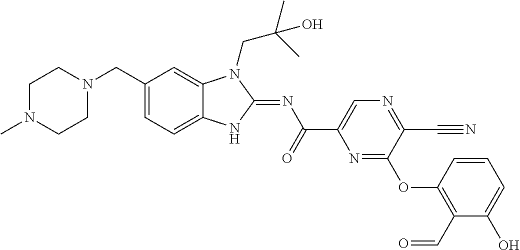 [SMILES: CN1CCN(Cc2ccc3[nH]/c(=N\C(=O)c4cnc(C#N)c(Oc5cccc(O)c5C=O)n4)n(CC(C)(C)O)c3c2)CC1] |
| 110 | 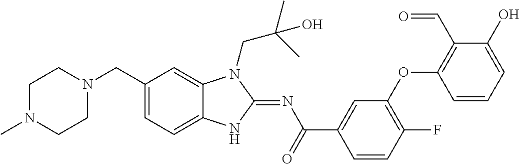 [SMILES: CN1CCN(Cc2ccc3[nH]/c(=N\C(=O)c4ccc(F)c(Oc5cccc(O)c5C=O)c4)n(CC(C)(C)O)c3c2)CC1] |
| 111 | 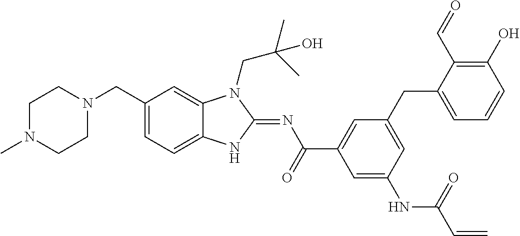 [SMILES: C=CC(=O)Nc1cc(Cc2cccc(O)c2C=O)cc(C(=O)/N=c2\[nH]c3ccc(CN4CCN(C)CC4)cc3n2CC(C)(C)O)c1] |
| 112 | 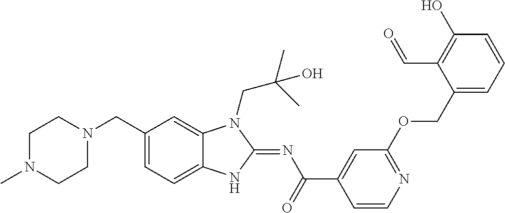 [SMILES: CN1CCN(Cc2ccc3[nH]/c(=N\C(=O)c4ccnc(OCc5cccc(O)c5C=O)c4)n(CC(C)(C)O)c3c2)CC1] |

In some aspects, the present disclosure provides compounds represented by the structure of Formula (VIIIA) or (VIIIB):

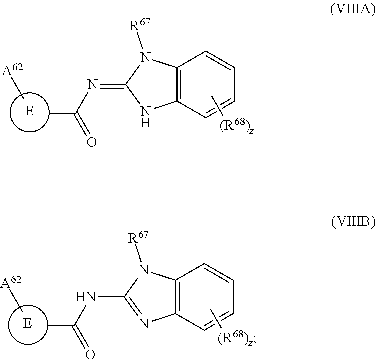

or a pharmaceutically acceptable salt thereof, wherein:
E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —OC(O)N(R⁷⁶)₂, —C(O)N(R⁷⁶)₂, —N(R⁷⁶)C(O)R⁷⁶, —N(R⁷⁶)C(O)OR⁷⁶, —N(R⁷⁶)C(O)N(R⁷⁶)₂, —N(R⁷⁶)S(O)₂(R⁷⁶), —S(O)R⁷⁶, —S(O)₂R⁷⁶, —S(O)₂N(R⁷⁶)₂, —NO₂, and —CN;
A⁶² is represented by *-L⁶²-C⁶², wherein * represents the connection to E;
   L⁶² is selected from a bond and -L^{G}-L^{H}-;
      L^{G} is selected from absent, —O—, —S—, and —N(R⁶⁹)—; and
      L^{H} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —OC(O)N(R⁷⁷)₂, —C(O)N(R⁷⁷)₂, —N(R⁷⁷)C(O)R⁷⁷, —N(R⁷⁷)C(O)OR⁷⁷, —N(R⁷⁷)C(O)N(R⁷⁷)₂, —N(R⁷⁷)S(O)₂(R⁷⁷), —S(O)R⁷⁷, —S(O)₂R⁷⁷, —S(O)₂N(R⁷⁷)₂, ═O, ═S, ═NR⁷⁷, —NO₂, and —CN;
   C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —OC(O)N(R⁷⁸)₂, —C(O)N(R⁷⁸)₂, —N(R⁷⁸)C(O)R⁷⁸, —N(R⁷⁸)C(O)OR⁷⁸, —N(R⁷⁸)C(O)N(R⁷⁸)₂, —N(R⁷⁸)S(O)₂(R⁷⁸), —S(O)R⁷⁸, —S(O)₂R⁷⁸, —S(O)₂N(R⁷⁸)₂, —NO₂, and —CN;
R⁶⁷ is independently selected from:
   C₁₋₆ alkyl, C₁₋₆ alkenyl, and C₁₋₆ alkynyl, each of which is optionally substituted with one or more substituents selected from:
      halogen, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN; and
      C₃₋₆ carbocycle, and 5- to 6-membered heterocycle, wherein the C₃₋₆ carbocycle and 5- to 6-membered heterocycle are each optionally substituted with one or more substituents selected from halogen, C₁₋₄ alkyl, C₁₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN;
   C₃₋₈ carbocycle and 3- to 8-membered heterocycle, each of which is optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN;
R⁶⁸ are each independently selected at each occurrence from halogen, C₁₋₆ alkyl, C₁₋₆ haloalkyl, —OR⁸⁰, —SR⁸⁰, —N(R⁸⁰)₂, —C(O)R⁸⁰, —C(O)OR⁸⁰, —OC(O)R⁸⁰, —C(O)N(R⁸⁰)₂, —N(R⁸⁰)S(O)₂(R⁸⁰), —S(O)R⁸⁰, —S(O)₂R⁸⁰, —S(O)₂N(R⁸⁰)₂, —NO₂, and —CN;
R⁶⁹ is selected from hydrogen, C₁₋₆ alkyl, and C₁₋₆ haloalkyl;
R⁷⁹ is selected at each occurrence from:
   hydrogen;
   C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸1, —OC(O)R⁸1, —C(O)N(R⁸¹)₂, —N(R⁸¹)C(O)R⁸1, —N(R⁸¹)S(O)₂(R⁸1), —S(O)R⁸¹, —S(O)₂R⁸¹, —S(O)₂N(R⁸¹)₂, ═O, ═S, —NO₂, and —CN;
   C₃₋₆ carbocycle and 5- to 6-membered heterocycle, each of which is optionally substituted with one or more substituents selected from halogen, C₁₋₄ alkyl, C₁₋₄ haloalkyl, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), —S(O)R⁸¹, —S(O)₂R⁸¹, —S(O)₂N(R⁸¹)₂, ═O, ═S, —NO₂, and —CN;
R⁷⁶, R⁷⁷, R⁷⁸, R⁸⁰, and R⁸¹ are each independently selected at each occurrence from hydrogen, C₁₋₆alkyl, and C₁₋₆ haloalkyl; and
z is selected from 0, 1, 2, and 3.

In some embodiments, the present disclosure provides compounds represented by the structure of Formula (VIIIA) or (VIIIB):

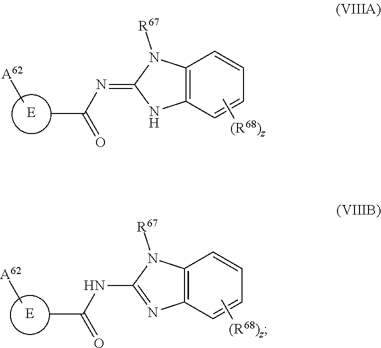

or a pharmaceutically acceptable salt thereof, wherein:
E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —OC(O)N(R⁷⁶)₂, —C(O)N(R⁷⁶)₂, —N(R⁷⁶)C(O)R⁷⁶, —N(R⁷⁶)C(O)OR⁷⁶, —N(R⁷⁶)C(O)N(R⁷⁶)₂, —N(R⁷⁶)S(O)₂(R⁷⁶), —NO₂, and —CN;
A⁶² is represented by *-L⁶²-C⁶², wherein * represents the connection to E;
   L⁶² is selected from a bond and -L^{G}-L^{H}-;
      L^{G} is selected from absent, —O—, —S—, and —N(R⁶⁹)—; and
      L^{H} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —OC(O)N(R⁷⁷)₂, —C(O)N(R⁷⁷)₂, —N(R⁷⁷)C(O)R⁷⁷, —N(R⁷⁷)C(O)OR⁷⁷, —N(R⁷⁷)C(O)N(R⁷⁷)₂, —N(R⁷⁷)S(O)₂(R⁷⁷), ═O, ═S, ═NR⁷⁷, —NO₂, and —CN;
   C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —OC(O)N(R⁷⁸)₂, —C(O)N(R⁷⁸)₂, —N(R⁷⁸)C(O)R⁷⁸, —N(R⁷⁸)C(O)OR⁷⁸, —N(R⁷⁸)C(O)N(R⁷⁸)₂, —N(R⁷⁸)S(O)₂(R⁷⁸), —NO₂, and —CN;
R⁶⁷ is independently selected from:
   C₁₋₆ alkyl, C₁₋₆ alkenyl, and C₁₋₆ alkynyl, each of which is optionally substituted with one or more substituents selected from halogen, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, ═S, —NO₂, and —CN;
      C₃₋₆ carbocycle, and 5- to 6-membered heterocycle, wherein the C₃₋₆ carbocycle and 5- to 6-membered heterocycle are each optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, ═S, —NO₂, and —CN;
   C₃₋₈ carbocycle and 3- to 8-membered heterocycle, each of which is optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, ═S, —NO₂, and —CN;
R⁶⁸ are each independently selected at each occurrence from halogen, C₁₋₆ alkyl, C₁₋₆ haloalkyl, —OR⁸⁰, —SR⁸⁰, —N(R⁸⁰)₂, —C(O)R⁸⁰, —C(O)OR⁸⁰, —OC(O)R⁸⁰, —C(O)N(R⁸⁰)₂, —N(R⁸⁰)S(O)₂(R⁸⁰), —NO₂, and —CN;
R⁶⁹ is selected from hydrogen, C₁₋₆ alkyl, and C₁₋₆ haloalkyl;
R⁷⁹ is selected at each occurrence from:
   hydrogen;
   C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸1, —OC(O)R⁸1, —C(O)N(R⁸¹)₂, —N(R⁸¹)C(O)R⁸1, —N(R⁸¹)S(O)₂(R⁸1), ═O, ═S, —NO₂, and —CN;
   C₃₋₆ carbocycle and 5- to 6-membered heterocycle, each of which is optionally substituted with one or more substituents selected from halogen, C₁₋₄ alkyl, C₁₋₄ haloalkyl, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), ═O, ═S, —NO₂, and —CN;
R⁷⁶, R⁷⁷, R⁷⁸, R⁸⁰, and R⁸¹ are each independently selected at each occurrence from hydrogen, C₁₋₆alkyl, and C₁₋₆ haloalkyl; and
z is selected from 0, 1, 2, and 3.

In some embodiments, the present disclosure provides compounds represented by the structure of Formula (VIIIA) or (VIIIB):

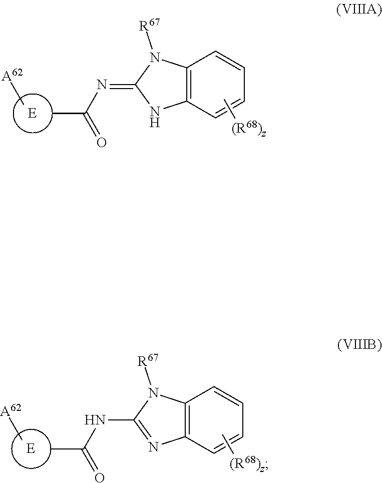

or a pharmaceutically acceptable salt thereof, wherein:
E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —C(O)N(R⁷⁶)₂, —N(R⁷⁶)C(O)R⁷⁶, —N(R⁷⁶)S(O)₂(R⁷⁶), —NO₂, and —CN;
A⁶² is represented by *-L⁶²-C⁶², wherein * represents the connection to E;
   L⁶² is selected from a bond and -L^{G}-L^{H}-;
      L^{G} is selected from absent, —O—, —S—, and —N(R⁶⁹)—; and
      L^{H} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —C(O)N(R⁷⁷)₂, —N(R⁷⁷)C(O)R⁷⁷, —N(R⁷⁷)S(O)₂(R⁷⁷), ═O, —NO₂, and —CN;
   C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —C(O)N(R⁷⁸)₂, —N(R⁷⁸)C(O)R⁷⁸, —N(R⁷⁸)S(O)₂(R⁷⁸), —NO₂, and —CN;
R⁶⁷ is independently selected from:
   C₁₋₆ alkyl, C₁₋₆ alkenyl, and C₁₋₆ alkynyl, each of which is optionally substituted with one or more substituents selected from halogen, —OR⁷⁹, —SR⁷⁹, —N(R⁷⁹)₂, —C(O)R⁷⁹, —C(O)OR⁷⁹, —OC(O)R⁷⁹, —C(O)N(R⁷⁹)₂, —N(R⁷⁹)C(O)R⁷⁹, —N(R⁷⁹)S(O)₂(R⁷⁹), and ═O; and
      C₃₋₆ carbocycle, and 5- to 6-membered heterocycle, wherein the C₃₋₆ carbocycle and 5- to 6-membered heterocycle are each optionally substituted with one or more substituents selected from halogen, C₁₋₄ alkyl, C₁₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, —NO₂, and —CN;
   C₃₋₈ carbocycle and 3- to 8-membered heterocycle, each of which is optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, —NO₂, and —CN;
R⁶⁸ are each independently selected at each occurrence from halogen, C₁₋₆ alkyl, C₁₋₆ haloalkyl, —OR⁸⁰, —SR⁸⁰, —N(R⁸⁰)₂, —C(O)R⁸⁰, —C(O)OR⁸⁰, —OC(O)R⁸⁰, —C(O)N(R⁸⁰)₂, —N(R⁸⁰)S(O)₂(R⁸⁰), —NO₂, and —CN;
R⁶⁹ is selected from hydrogen, C₁₋₆ alkyl, and C₁₋₆ haloalkyl;
R⁷⁹ is selected at each occurrence from:
hydrogen;
C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸1, —OC(O)R⁸1, —C(O)N(R⁸¹)₂, —N(R⁸¹)C(O)R⁸1, —N(R⁸¹)S(O)₂(R⁸1), and ═O;
   C₃₋₆ carbocycle and 5- to 6-membered heterocycle, each of which is optionally substituted with one or more substituents selected from halogen, C₁₋₄ alkyl, C₁₋₄ haloalkyl, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), ═O, —NO₂, and —CN;
R⁷⁶, R⁷⁷, R⁷⁸, R⁸⁰, and R⁸¹ are each independently selected at each occurrence from hydrogen, C₁₋₆alkyl, and C₁₋₆ haloalkyl; and
z is selected from 0, 1, 2, and 3.

In some embodiments, the present disclosure provides compounds represented by the structure of Formula (VIIIA) or (VIIIB):

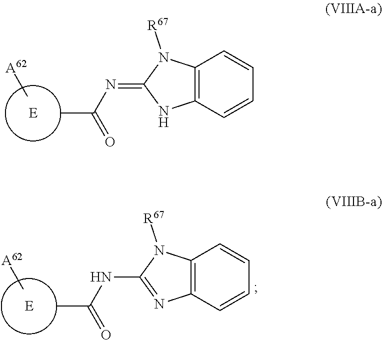

or a pharmaceutically acceptable salt thereof, wherein:
E is phenyl or 5- to 10-membered heteroaryl optionally substituted with one or more substituents independently selected from: C₁₋₆ alkyl;
A⁶² is represented by *-L⁶²-C⁶², wherein * represents the connection to E;
   L⁶² is selected from a bond and -L^{G}-L^{H}-;
      L^{G} is selected from absent, —O—, and —N(R⁶⁹)—; and
      L^{H} is selected from absent, C₁₋₄ alkylene, C₂₋₄ alkynylene, phenylene, and 4- to 6-membered heterocyclene;
   C⁶² is phenyl substituted with —C(O)H and optionally substituted with one or more substituents independently selected from —OR⁷⁸;
R⁶⁷ is independently selected from:
   C₁₋₆ alkyl optionally substituted with one or more substituents selected from —OR⁷⁹; and
      5- to 6-membered heterocycle optionally substituted with one or more substituents selected from C₁₋₄ alkyl;
   C₃₋₈ carbocycle and 3- to 8-membered heterocycle, each of which is optionally substituted with one or more substituents selected from: —OR⁷⁹ and —C(O)R⁷⁹;
R⁶⁹ is hydrogen;
R⁷⁹ is selected at each occurrence from:
   hydrogen;
   C₁₋₆ alkenyl optionally substituted with one or more substituents selected from —N(R⁸¹)₂; and
R⁷⁶, R⁷⁷, R⁷⁸, R⁸⁰, and R⁸¹ are each independently selected at each occurrence from hydrogen and C₁₋₆ alkyl.

In some embodiments, E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —OC(O)N(R⁷⁶)₂, —C(O)N(R⁷⁶)₂, —N(R⁷⁶)C(O)R⁷⁶, —N(R⁷⁶)C(O)OR⁷⁶, —N(R⁷⁶)C(O)N(R⁷⁶)₂, —N(R⁷⁶)S(O)₂(R⁷⁶), —S(O)R⁷⁶, —S(O)₂R⁷⁶, —S(O)₂N(R⁷⁶)₂, —NO₂, and —CN. In some embodiments, E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —OC(O)N(R⁷⁶)₂, —C(O)N(R⁷⁶)₂, —N(R⁷⁶)C(O)R⁷⁶, —N(R⁷⁶)C(O)OR⁷⁶, —N(R⁷⁶)C(O)N(R⁷⁶)₂, —N(R⁷⁶)S(O)₂(R⁷⁶), —NO₂, and —CN. In some embodiments, E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —C(O)N(R⁷⁶)₂, —N(R⁷⁶)C(O)R⁷⁶, —N(R⁷⁶)S(O)₂(R⁷⁶), —NO₂, and —CN. In some embodiments, E is phenyl or 5- to 10-membered heteroaryl optionally substituted with one or more substituents independently selected from: halogen, C₁₋₆ alkyl, C₁₋₆ haloalkyl, —OR⁷⁰, —N(R⁷⁰)₂, —C(O)R⁷⁰, —C(O)OR⁷⁰, —C(O)N(R⁷⁰)₂, —N(R⁷⁰)C(O)R⁷⁰, and —CN. In some embodiments, E is phenyl or 5- to 10-membered heteroaryl optionally substituted with one or more substituents independently selected from: C₁₋₆ alkyl. In some embodiments, E is phenyl or pyridyl, optionally substituted with one or more substituents independently selected from: halogen, C₁₋₆ alkyl, C₁₋₆ haloalkyl, —OR⁷⁰, —N(R⁷⁰)₂, —C(O)R⁷⁰, —C(O)OR⁷⁰, —C(O)N(R⁷⁰)₂, —N(R⁷⁰)C(O)R⁷⁰, and —CN. In some embodiments, E is phenyl or pyridyl, optionally substituted with one or more substituents independently selected from C₁₋₆ alkyl.

In some embodiments, the compound is represented by the structure of Formula (IXA) or (IXB):

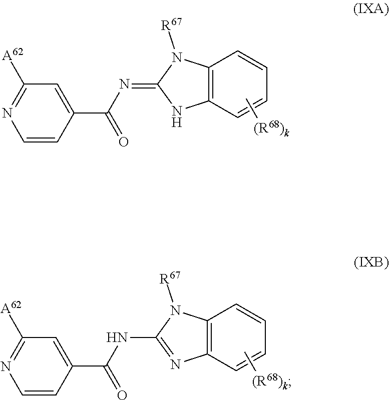

or a pharmaceutically acceptable salt thereof.

In some embodiments, L⁶² is selected from a bond and -L^{G}-L^{H}-. In some embodiments, L⁶² is a bond. In some embodiments, L⁶² is -L^{G}-L^{H}-.

In some embodiments, L^{G} is selected from absent, —O—, —S—, and —N(R⁶⁹)—. In some embodiments, L^{G} is selected from absent, —O—, and —N(R⁶⁹)—. In some embodiments, L^{G} is selected from absent and —O—. In some embodiments, L^{G} is selected from absent and —N(R⁶⁹)—.

In some embodiments, L^{G} is selected from —O— and —N(R⁶⁹)—. In some embodiments, L^{G} is absent. In some embodiments, L^{G} is —O—. In some embodiments, L^{G} is —S—. In some embodiments, L^{G} is —N(R⁶⁹)—.

In some embodiments, L^{H} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —OC(O)N(R⁷⁷)₂, —C(O)N(R⁷⁷)₂, —N(R⁷⁷)C(O)R⁷⁷, —N(R⁷⁷)C(O)OR⁷⁷, —N(R⁷⁷)C(O)N(R⁷⁷)₂, —N(R⁷⁷)S(O)₂(R⁷⁷), —S(O)R⁷⁷, —S(O)₂R⁷⁷, —S(O)₂N(R⁷⁷)₂, ═O, ═S, ═NR⁷⁷, —NO₂, and —CN. In some embodiments, L^{H} is selected from absent, C₁₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —OC(O)N(R⁷⁷)₂, —C(O)N(R⁷⁷)₂, —N(R⁷⁷)C(O)R⁷⁷, —N(R⁷⁷)C(O)OR⁷⁷, —N(R⁷⁷)C(O)N(R⁷⁷)₂, —N(R⁷⁷)S(O)₂(R⁷⁷), ═O, ═S, ═NR⁷⁷, —NO₂, and —CN. In some embodiments, L^{H} is selected from absent, C₁₋₄ alkylene, C₂-4 alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —C(O)N(R⁷⁷)₂, —N(R⁷⁷)C(O)R⁷⁷, —N(R⁷⁷)S(O)₂(R⁷⁷), ═O, —NO₂, and —CN. In some embodiments, L^{H} is selected from absent, C₁₄ alkylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —C(O)N(R⁷⁷)₂, —N(R⁷⁷)C(O)R⁷⁷, ═O, and —CN. In some embodiments, L^{H} is selected from absent, C₁₋₄ alkylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, and ═O. In some embodiments, L^{H} is selected from absent, C₁₋₄ alkylene, C₂₋₄ alkynylene, phenylene, and 4- to 6-membered heterocyclene. In some embodiments, L^{H} is selected from absent, methylene, ethylene, ethynylene, phenylene, piperazinylene, and pyrazolylene.

In some embodiments, L⁶² is selected from a bond, —O—, —OCH₂—, —N(H)—, ethylene, ethynylene,

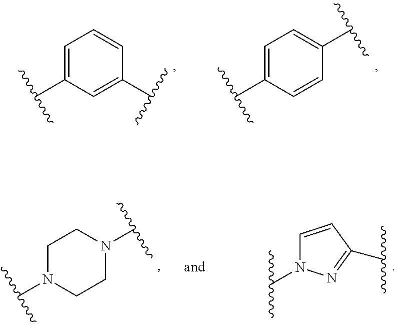 [SMILES: *c1cccc(*)c1 ; *c1ccc(*)cc1 ; *N1CCN(*)CC1 ; *c1ccn(*)n1]

In some embodiments, C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —OC(O)N(R⁷⁸)₂, —C(O)N(R⁷⁸)₂, —N(R⁷⁸)C(O)R⁷⁸, —N(R⁷⁸)C(O)OR⁷⁸, —N(R⁷⁸)C(O)N(R⁷⁸)₂, —N(R⁷⁸)S(O)₂(R⁷⁸), —S(O)R⁷⁸, —S(O)₂R⁷⁸, —S(O)₂N(R⁷⁸)₂, —NO₂, and —CN. In some embodiments, C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —OC(O)N(R⁷⁸)₂, —C(O)N(R⁷⁸)₂, —N(R⁷⁸)C(O)R⁷⁸, —N(R⁷⁸)C(O)OR⁷⁸, —N(R⁷⁸)C(O)N(R⁷⁸)₂, —N(R⁷⁸)S(O)₂(R⁷⁸), —NO₂, and —CN. In some embodiments, C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —C(O)N(R⁷⁸)₂, —N(R⁷⁸)C(O)R⁷⁸, —N(R⁷⁸)S(O)₂(R⁷⁸), —NO₂, and —CN. In some embodiments, C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —N(R⁷⁸)C(O)R⁷⁸, —N(R⁷⁸)C(O)OR⁷⁸, —N(R⁷⁸)C(O)N(R⁷⁸)₂, —N(R⁷⁸)S(O)₂(R⁷⁸), —NO₂, and —CN. In some embodiments, C⁶² is selected from phenyl and monocyclic 5- to 6-membered heteroaryl, any of which is substituted with —C(O)H and optionally substituted with one or more substituents independently selected from: halogen, —OR⁷⁸, —N(R⁷⁸)₂, —N(R⁷⁸)C(O)R⁷⁸, —N(R⁷⁸)C(O)OR⁷⁸, and —N(R⁷⁸)S(O)₂(R⁷⁸). In some embodiments, C⁶² is phenyl substituted with —C(O)H and optionally substituted with one or more substituents independently selected from —OR⁷⁸. In some embodiments, C⁶² is selected from phenyl and monocyclic 5- to 6-membered heteroaryl, any of which is substituted with —C(O)H and —OH. In some embodiments, C⁶² is selected from:

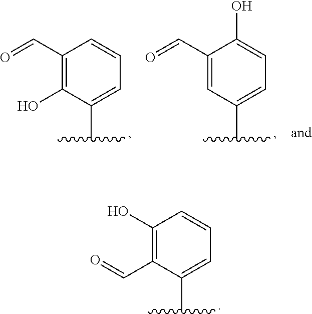

In some embodiments, R⁶⁷ is independently selected from:
C₁₋₆ alkyl, C₁₋₆ alkenyl, and C₁₋₆ alkynyl, each of which is optionally substituted with one or more substituents selected from:
   halogen, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN; and
   C₃₋₆ carbocycle, and 5- to 6-membered heterocycle, wherein the C₃₋₆ carbocycle and 5 to 6-membered heterocycle are each optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN; and
C₃₋₈ carbocycle and 3- to 8-membered heterocycle, each of which is optionally substituted with one or more substituents selected from halogen, C₁₋₄ alkyl, C₁₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN.

In some embodiments, R⁶⁷ is independently selected from:
C₁₋₆ alkyl, C₁₋₆ alkenyl, and C₁₋₆ alkynyl, each of which is optionally substituted with one or more substituents selected from halogen, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, ═S, —NO₂, and —CN;
   C₃₋₆ carbocycle, and 5- to 6-membered heterocycle, wherein the C₃₋₆ carbocycle and 5 to 6-membered heterocycle are each optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, ═S, —NO₂, and —CN; and
C₃₋₈ carbocycle and 3- to 8-membered heterocycle, each of which is optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, ═S, —NO₂, and —CN.

In some embodiments, R⁶⁷ is independently selected from:
C₁₋₆ alkyl, C₁₋₆ alkenyl, and C₁₋₆ alkynyl, each of which is optionally substituted with one or more substituents selected from halogen, —OR⁷⁹, —SR⁷⁹, —N(R⁷⁹)₂, —C(O)R⁷⁹, —C(O)OR⁷⁹, —OC(O)R⁷⁹, —C(O)N(R⁷⁹)₂, —N(R⁷⁹)C(O)R⁷⁹, —N(R⁷⁹)S(O)₂(R⁷⁹), and ═O; and
   C₃₋₆ carbocycle, and 5- to 6-membered heterocycle, wherein the C₃₋₆ carbocycle and 5 to 6-membered heterocycle are each optionally substituted with one or more substituents selected from halogen, C₁₋₄ alkyl, C₁₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, —NO₂, and —CN; and
C₃₋₈ carbocycle and 3- to 8-membered heterocycle, each of which is optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), ═O, —NO₂, and —CN.

In some embodiments, R⁶⁷ is independently selected from:
C₁₋₆ alkyl, C₁₋₆ alkenyl, and C₁₋₆ alkynyl, each of which is optionally substituted with one or more substituents selected from halogen, —OR⁷⁹, —SR⁷⁹, —N(R⁷⁹)₂, —C(O)N(R⁷⁹)₂, and —N(R⁷⁹)C(O)R⁷⁹; and
   C₃₋₆ carbocycle, and 5- to 6-membered heterocycle, wherein the C₃₋₆ carbocycle and 5 to 6-membered heterocycle are each optionally substituted with one or more substituents selected from halogen, C₁₋₄ alkyl, C₁₄ haloalkyl, —OR⁷⁹, —N(R⁷⁹)₂, ═O, NO₂, and —CN; and
C₃₋₈ carbocycle and 3- to 8-membered heterocycle, each of which is optionally substituted with one or more substituents selected from halogen, C₁₋₄ alkyl, C₁₋₄ haloalkyl, —OR⁷⁹, —SR⁷⁹, —N(R⁷⁹)₂, —C(O)R⁷⁹, —C(O)OR⁷⁹, —C(O)N(R⁷⁹)₂, —N(R⁷⁹)C(O)R⁷⁹, ═O, —NO₂, and —CN.

In some embodiments, R⁶⁷ is independently selected from:
C₁₋₆ alkyl optionally substituted with one or more substituents selected from —OR⁷⁹; and
   5- to 6-membered heterocycle optionally substituted with one or more substituents selected from C₁₋₄ alkyl; and
   C₃₋₈ carbocycle and 3- to 8-membered heterocycle, each of which is optionally substituted with one or more substituents selected from: —OR⁷⁹ and —C(O)R⁷⁹.

In some embodiments, R⁷⁹ is selected at each occurrence from:
hydrogen;
C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸¹, —OC(O)R⁸¹, —C(O)N(R⁸1)₂, —N(R⁸¹)C(O)R⁸¹, —N(R⁸¹)S(O)₂(R⁸¹), —S(O)R⁸1, —S(O)₂R⁸¹, —S(O)₂N(R⁸¹)₂, ═O, ═S, —NO₂, and —CN; and
C₃₋₆ carbocycle and 5- to 6-membered heterocycle, each of which is optionally substituted with one or more substituents selected from halogen, C₁₋₄ alkyl, C₁₄ haloalkyl, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), —S(O)R⁸¹, —S(O)₂R⁸¹, —S(O)₂N(R⁸¹)₂, ═O, ═S, —NO₂, and —CN.

In some embodiments, R⁷⁹ is selected at each occurrence from:
hydrogen;
C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), ═O, ═S, —NO₂, and —CN; and
C₃₋₆ carbocycle and 5- to 6-membered heterocycle, each of which is optionally substituted with one or more substituents selected from halogen, C₁₋₄ alkyl, C₁₄ haloalkyl, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), ═O, ═S, —NO₂, and —CN.

In some embodiments, R⁷⁹ is selected at each occurrence from:
hydrogen;
C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸¹, —OC(O)R⁸¹, —C(O)N(R⁸1)₂, —N(R⁸¹)C(O)R⁸¹, —N(R⁸¹)S(O)₂(R⁸¹), and ═O; and
C₃₋₆ carbocycle and 5- to 6-membered heterocycle, each of which is optionally substituted with one or more substituents selected from halogen, C₁₋₄ alkyl, C₁₋₄ haloalkyl, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), ═O, —NO₂, and —CN.

In some embodiments, R⁷⁹ is selected at each occurrence from:
hydrogen; and
C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸¹, —OC(O)R⁸¹, —C(O)N(R⁸1)₂, —N(R⁸¹)C(O)R⁸¹, —N(R⁸¹)S(O)₂(R⁸¹), —S(O)R⁸1, —S(O)₂R⁸¹, —S(O)₂N(R⁸¹)₂, ═O, —NO₂, and —CN.

In some embodiments, R⁷⁹ is selected at each occurrence from:
hydrogen; and
C₁₋₆ alkyl, C₁₋₆ alkenyl, and C₁₋₆ alkynyl each of which is optionally substituted with one or more substituents selected from halogen, —OR⁸¹, —N(R⁸¹)₂, —C(O)N(R⁸¹)₂, —N(R⁸¹)C(O)R⁸¹, and —CN.

In some embodiments, R⁷⁹ is selected at each occurrence from: hydrogen and C₁₋₆ alkenyl optionally substituted with one or more substituents selected from halogen, —OR⁸¹, —N(R⁸¹)₂, —C(O)N(R⁸¹)₂, —N(R⁸¹)C(O)R⁸¹, and —CN. In some embodiments, R⁷⁹ is selected at each occurrence from: hydrogen and C₁₋₆ alkenyl optionally substituted with one or more substituents selected from —N(R⁸¹)₂.

In some embodiments, R⁶⁷ is independently selected from: methyl,

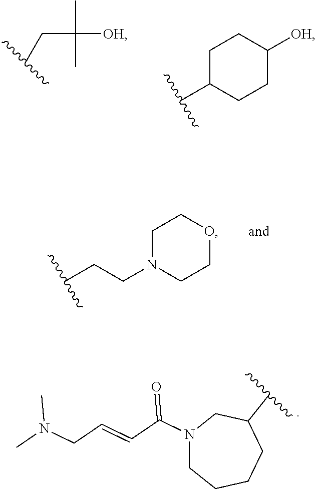

In some embodiments, z is selected from 0, 1, 2, and 3. In some embodiments, z is selected from 0, 1, and 2. In some embodiments, z is selected from 0 and 1. In some embodiments, z is 0. In some embodiments, z is 1. In some embodiments, z is 2. In some embodiments, z is 3.

In some embodiments, R⁷⁶, R⁷⁷, R⁷⁸, R⁸⁰, and R⁸¹ are each independently selected at each occurrence from hydrogen, C₁₋₆ alkyl, and C₁₋₆ haloalkyl. In some embodiments, R⁷⁶, R⁷⁷, R⁷⁸, R⁸⁰, and R⁸¹ are each independently selected at each occurrence from hydrogen and C₁₋₆ alkyl. In some embodiments, R⁷⁶, R⁷⁷, R⁷⁸, R⁸⁰, and R⁸¹ are each independently selected at each occurrence from hydrogen and methyl. In some embodiments, R⁷⁶, R⁷⁷, R⁷⁸, R⁸⁰, and R⁸¹ are each independently selected at each occurrence from hydrogen. In some embodiments, R⁷⁶, R⁷⁷, R⁷⁸, R⁸⁰, and R⁸¹ are each independently selected at each occurrence from C₁₋₆ alkyl. In some embodiments, R⁷⁶, R⁷⁷, R⁷⁸, R⁸⁰, and R⁸¹ are each independently selected at each occurrence from methyl.

In some embodiments, R⁷⁶ is independently selected at each occurrence from hydrogen, C₁₋₆ alkyl, and C₁₋₆ haloalkyl. In some embodiments, R⁷⁶ is independently selected at each occurrence from hydrogen and C₁₋₆ alkyl. In some embodiments, R⁷⁶ is independently selected at each occurrence from hydrogen and methyl. In some embodiments, R⁷⁶ is independently selected at each occurrence from hydrogen. In some embodiments, R⁷⁶ is independently selected at each occurrence from C₁₋₆ alkyl. In some embodiments, R⁷⁶ is methyl.

In some embodiments, R⁷⁷ is independently selected at each occurrence from hydrogen, C₁₋₆ alkyl, and C₁₋₆ haloalkyl. In some embodiments, R⁷⁷ is independently selected at each occurrence from hydrogen and C₁₋₆ alkyl. In some embodiments, R⁷⁷ is independently selected at each occurrence from hydrogen and methyl. In some embodiments, R⁷⁷ is independently selected at each occurrence from hydrogen. In some embodiments, R⁷⁷ is independently selected at each occurrence from C₁₋₆ alkyl. In some embodiments, R⁷⁷ is methyl.

In some embodiments, R⁷⁸ is independently selected at each occurrence from hydrogen, C₁₋₆ alkyl, and C₁₋₆ haloalkyl. In some embodiments, R⁷⁸ is independently selected at each occurrence from hydrogen and C₁₋₆ alkyl. In some embodiments, R⁷⁸ is independently selected at each occurrence from hydrogen and methyl. In some embodiments, R⁷⁸ is independently selected at each occurrence from hydrogen. In some embodiments, R⁷⁸ is independently selected at each occurrence from C₁₋₆ alkyl. In some embodiments, R⁷⁸ is methyl.

In some embodiments, R⁸⁰ is independently selected at each occurrence from hydrogen, C₁₋₆ alkyl, and C₁₋₆ haloalkyl. In some embodiments, R⁸⁰ is independently selected at each occurrence from hydrogen and C₁₋₆ alkyl. In some embodiments, R⁸⁰ is independently selected at each occurrence from hydrogen and methyl. In some embodiments, R⁸⁰ is independently selected at each occurrence from hydrogen. In some embodiments, R⁸⁰ is independently selected at each occurrence from C₁₋₆ alkyl. In some embodiments, R⁸⁰ is methyl.

In some embodiments, R⁸¹ is independently selected at each occurrence from hydrogen, C₁₋₆ alkyl, and C₁₋₆ haloalkyl. In some embodiments, R⁸¹ is independently selected at each occurrence from hydrogen and C₁₋₆ alkyl. In some embodiments, R⁸¹ is independently selected at each occurrence from hydrogen and methyl. In some embodiments, R⁸¹ is independently selected at each occurrence from hydrogen. In some embodiments, R⁸¹ is independently selected at each occurrence from C₁₋₆ alkyl. In some embodiments, R⁸¹ is methyl.

In some embodiments, the compound is selected from a compound of Table 4, or a pharmaceutically acceptable salt thereof.

TABLE 4

| Cmpd. | |
|---|---|
| No. | Structure |
| 121 | 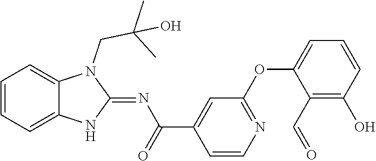 [SMILES: CC(C)(O)Cn1/c(=N/C(=O)c2ccnc(Oc3cccc(O)c3C=O)c2)[nH]c2ccccc21] |
| 122 | 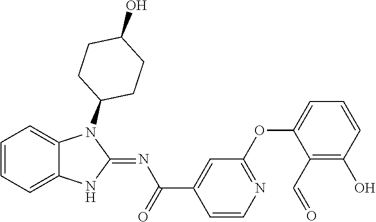 [SMILES: O=Cc1c(O)cccc1Oc1cc(C(=O)/N=c2\[nH]c3ccccc3n2[C@H]2CC[C@@H](O)CC2)ccn1] |
| 123 | 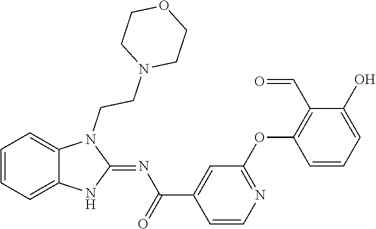 [SMILES: O=Cc1c(O)cccc1Oc1cc(C(=O)/N=c2\[nH]c3ccccc3n2CCN2CCOCC2)ccn1] |
| 124 | 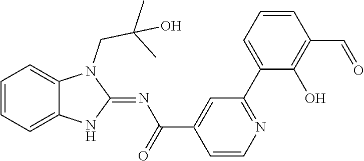 [SMILES: CC(C)(O)Cn1/c(=N/C(=O)c2ccnc(-c3cccc(C=O)c3O)c2)[nH]c2ccccc21] |
| 125 | 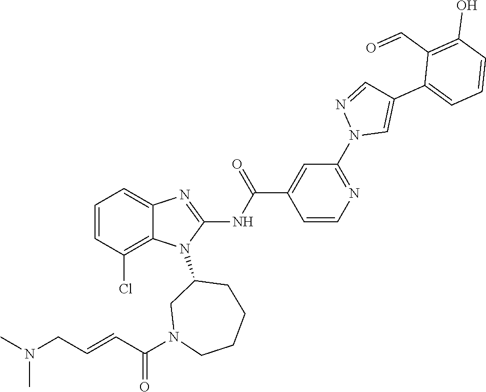 [SMILES: CN(C)C/C=C/C(=O)N1CCCC[C@@H](n2c(NC(=O)c3ccnc(-n4cc(-c5cccc(O)c5C=O)cn4)c3)nc3cccc(Cl)c32)C1] |
| 126 | 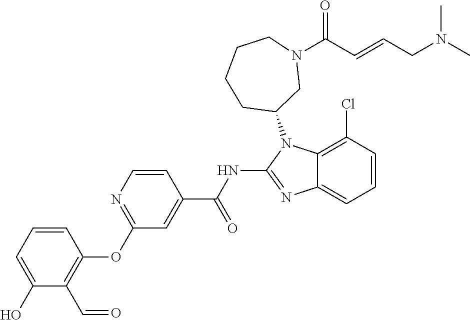 [SMILES: CN(C)C/C=C/C(=O)N1CCCC[C@@H](n2c(NC(=O)c3ccnc(Oc4cccc(O)c4C=O)c3)nc3cccc(Cl)c32)C1] |
| 127 | 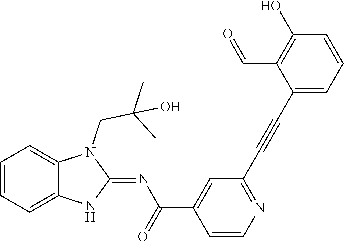 [SMILES: CC(C)(O)Cn1/c(=N/C(=O)c2ccnc(C#Cc3cccc(O)c3C=O)c2)[nH]c2ccccc21] |
| 128 | 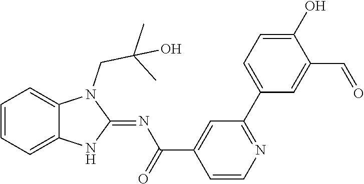 [SMILES: CC(C)(O)Cn1/c(=N/C(=O)c2ccnc(-c3ccc(O)c(C=O)c3)c2)[nH]c2ccccc21] |
| 129 | 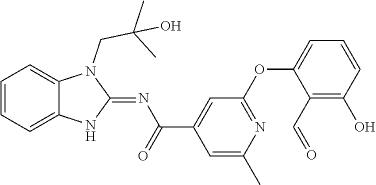 [SMILES: Cc1cc(C(=O)/N=c2\[nH]c3ccccc3n2CC(C)(C)O)cc(Oc2cccc(O)c2C=O)n1] |
| 130 | 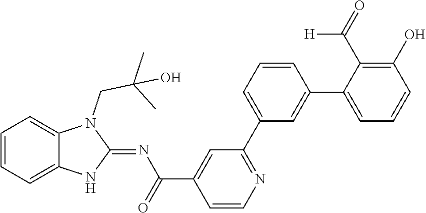 [SMILES: CC(C)(O)Cn1/c(=N/C(=O)c2ccnc(-c3cccc(-c4cccc(O)c4C=O)c3)c2)[nH]c2ccccc21] |
| 131 | 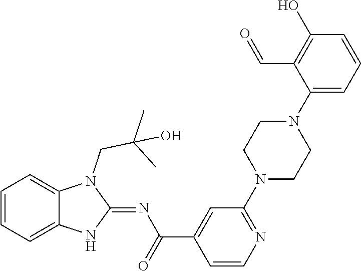 [SMILES: CC(C)(O)Cn1/c(=N/C(=O)c2ccnc(N3CCN(c4cccc(O)c4C=O)CC3)c2)[nH]c2ccccc21] |
| 132 | 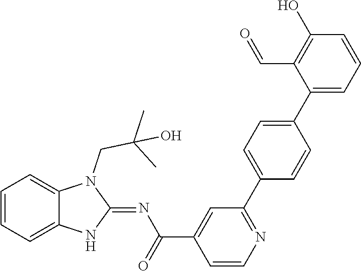 [SMILES: CC(C)(O)Cn1/c(=N/C(=O)c2ccnc(-c3ccc(-c4cccc(O)c4C=O)cc3)c2)[nH]c2ccccc21] |
| 133 | 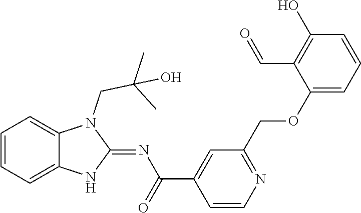 [SMILES: CC(C)(O)Cn1/c(=N/C(=O)c2ccnc(COc3cccc(O)c3C=O)c2)[nH]c2ccccc21] |
| 134 | 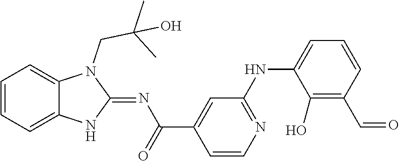 [SMILES: CC(C)(O)Cn1/c(=N/C(=O)c2ccnc(Nc3cccc(C=O)c3O)c2)[nH]c2ccccc21] |
| 135 | 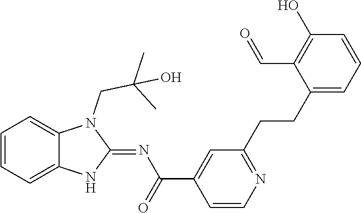 [SMILES: CC(C)(O)Cn1/c(=N/C(=O)c2ccnc(CCc3cccc(O)c3C=O)c2)[nH]c2ccccc21] |
| 136 | 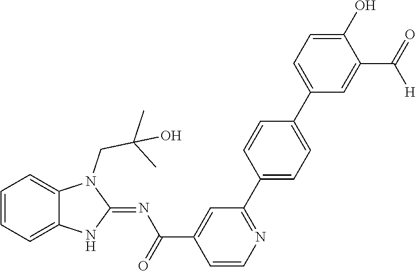 [SMILES: CC(C)(O)Cn1/c(=N/C(=O)c2ccnc(-c3ccc(-c4ccc(O)c(C=O)c4)cc3)c2)[nH]c2ccccc21] |
| 137 | 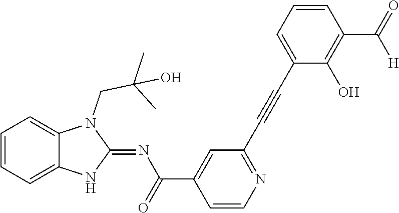 [SMILES: CC(C)(O)Cn1/c(=N/C(=O)c2ccnc(C#Cc3cccc(C=O)c3O)c2)[nH]c2ccccc21] |
| 138 | 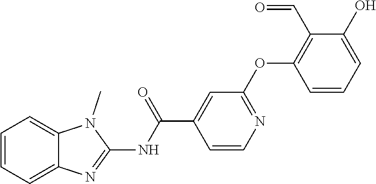 [SMILES: Cn1c(NC(=O)c2ccnc(Oc3cccc(O)c3C=O)c2)nc2ccccc21] |
| 139 | 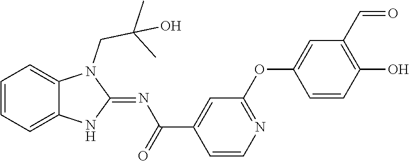 [SMILES: CC(C)(O)Cn1/c(=N/C(=O)c2ccnc(Oc3ccc(O)c(C=O)c3)c2)[nH]c2ccccc21] |
| 140 | 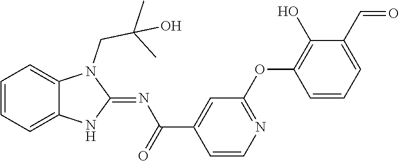 [SMILES: CC(C)(O)Cn1/c(=N/C(=O)c2ccnc(Oc3cccc(C=O)c3O)c2)[nH]c2ccccc21] |

In some aspects, the present disclosure provides a pharmaceutical composition comprising a compound or salt disclosed herein and at least one pharmaceutically acceptable excipient.

In some aspects, the present disclosure provides a method of inhibiting EGFR in a subject in need thereof, comprising administering to the subject a compound or salt disclosed herein or a pharmaceutical composition disclosed herein.

In some aspects, the present disclosure provides a method of treating cancer in a subject in need thereof, comprising administering to the subject a compound or salt disclosed herein or a pharmaceutical composition disclosed herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E- form (or cis- or trans- form). Furthermore, some chemical entities may exist in various tautomeric forms. Unless otherwise specified, compounds or salts of Formula (I), are intended to include all Z—, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I), herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I), may comprise two or more enantiomers or diatereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

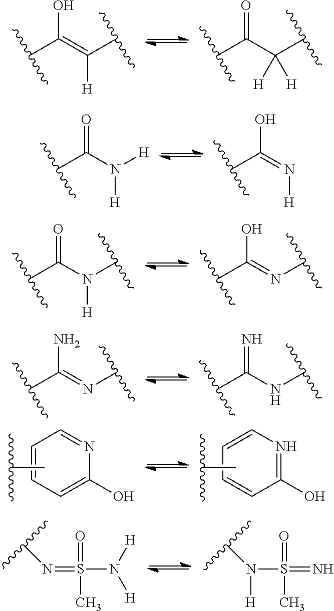

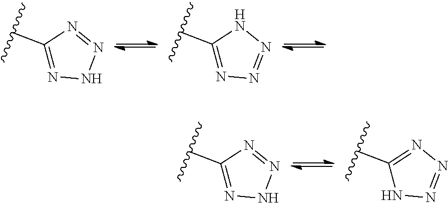 [SMILES: *c1nn[nH]n1 ; *c1nnn[nH]1 ; *c1nn[nH]n1 ; *c1nnn[nH]1]

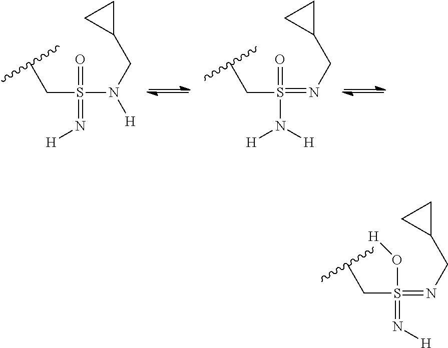 [SMILES: *CS(=N)(=O)NCC1CC1 ; *CS(N)(=O)=NCC1CC1 ; *CS(=N)(O)=NCC1CC1]

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (1). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formula (I) include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formula (I), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of compounds represented by Formula (I). The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

In certain embodiments, compounds or salts of Formula (I), may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser andFieser's Reagentsfor Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (I) and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Methods for formulation of the conjugates can include formulating any of the compounds, salts or conjugates with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates can be lyophilized or in powder form for re-constitution with a suitable vehicle, e.g., sterile pyrogen-free water, before use.

A compound or salt of Formula (I) may be formulated in any suitable pharmaceutical formulation. A pharmaceutical formulation of the present disclosure typically contains an active ingredient (e.g., compound or salt of any one of Formula (I)), and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

Pharmaceutical compositions may also be prepared from a compound or salt of any one of Formula (I) and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some aspects, the present disclosure provides a method for treatment, comprising administering to a subject in need thereof an effective amount of a compound or salt of Formula (I)

In certain embodiments, the present disclosure can be used as a method of inhibiting EGFR in a subject in need thereof, comprising administering to the subject a compound or salt of Formula (I) or a pharmaceutical composition of Formula (I).

### EXAMPLES

The invention now being generally described, it will be more readily understood by reference to the following examples which are included merely for purposes of illustration of certain aspects and embodiments of the present invention, and are not intended to limit the invention in any way.

The compounds disclosed herein may be tested using methods as described in Kashima K. et al. Mol Cancer Ther November 2020, 19(11), pp. 2288-2297; Akamatsu, H. et al. JAMA Oncol. 2021, 7(3), pp. 386-394; Angulo, B. et al. PLOS ONE 2012, 7(8), e43842; U.S. Ser. No. 15/910,035; and US U.S. Ser. No. 15/223,343, the entire contents of each of which are incorporated herein by reference.

### Example 1: General Synthetic Schemes

The following examples describe illustrate various methods of preparation of compounds described herein. Examples are exemplary and not exhaustive. It is understood that one skilled in the art may be able to synthesize described compounds by similar methods.

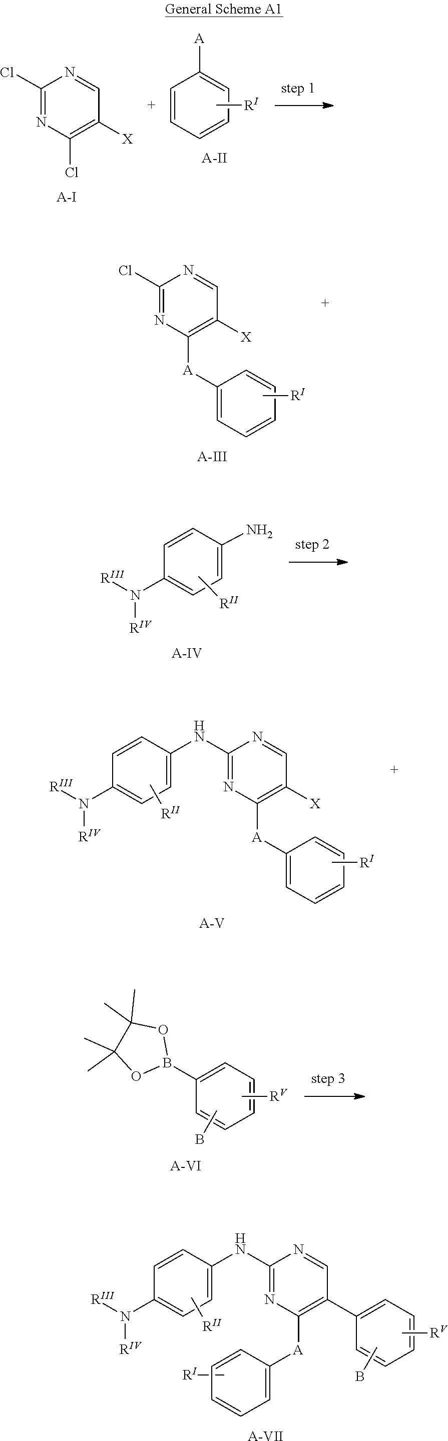

General Scheme A1 provides a general synthesis of 2-arylaminopyrimidines. In Schemes disclosed herein “X” is a halogen and can be chosen from Cl, Br, or I. “A” can be a heteroatom such as N, O, or S. “B” is an amine reactive electrophile such as an aldehyde that is optionally masked with a protecting group. In Step 1, 2,4,5-trihalopyridimine A-I can readily undergo a regioselective nucleophilic aromatic substitution with nucleophile A-II and an appropriate base to provide aminophenylpyrimidine A-III when (A=N), or phenoxypyrimidine A-III (A=O). In Step 2, substituted halopyridimine A-III can undergo a second regioselective nucleophilic aromatic substitution with aniline A-IV to provide 2-arylaminopyrimidine V. In Step 3, 2-arylaminopyrimidine A-V can be reacted with aryl boronic ester or aryl boronic acid A-VI via Suzuki cross coupling reaction to provide 2-arylaminopyrimidines A-VII. In some cases, the aryl boronic ester or ester A-VI can contain an aldehyde “B”. The aldehyde of boronic acid or ester A-VI can also be protected with a suitable protecting group such as an acetal with ethylene glycol, prior to Suzuki coupling. In this case, the protected aldehyde A-VII can be unmasked with acid such as trifluoroacetic acid or methanesulfonic acid to provide the desired 2-arylaminopyrimidines A-VII.

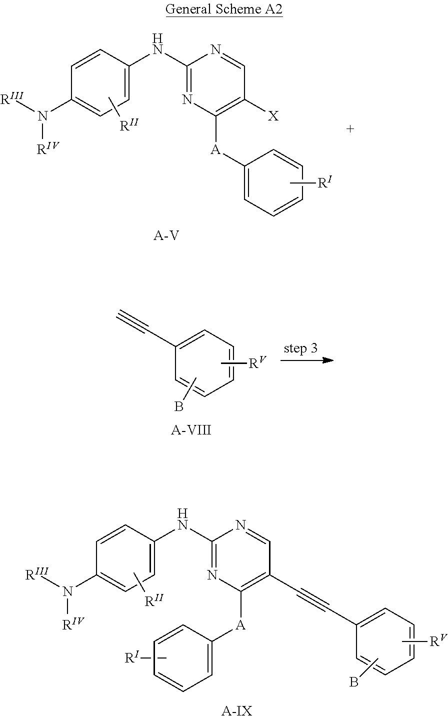

General Scheme A2 provides a general synthesis of Alkynylpyrimidine A-IX. 2-arylaminopyrimidine A-V can undergo a Sonogashira coupling with alkyne A-VIII to generate Alkynylpyrimidine A-IX. In some cases, alkyne A-VIII can contain an aldehyde “B”. The aldehyde of alkyne VIII can also be protected with a suitable protecting group such as an acetal with ethylene glycol, prior to Sonogashira coupling. In this case, the protected aldehyde A-VIII can be unmasked with acid such as trifluoroacetic acid or methanesulfonic acid to provide the desired 2-arylaminopyrimidines A-IX.

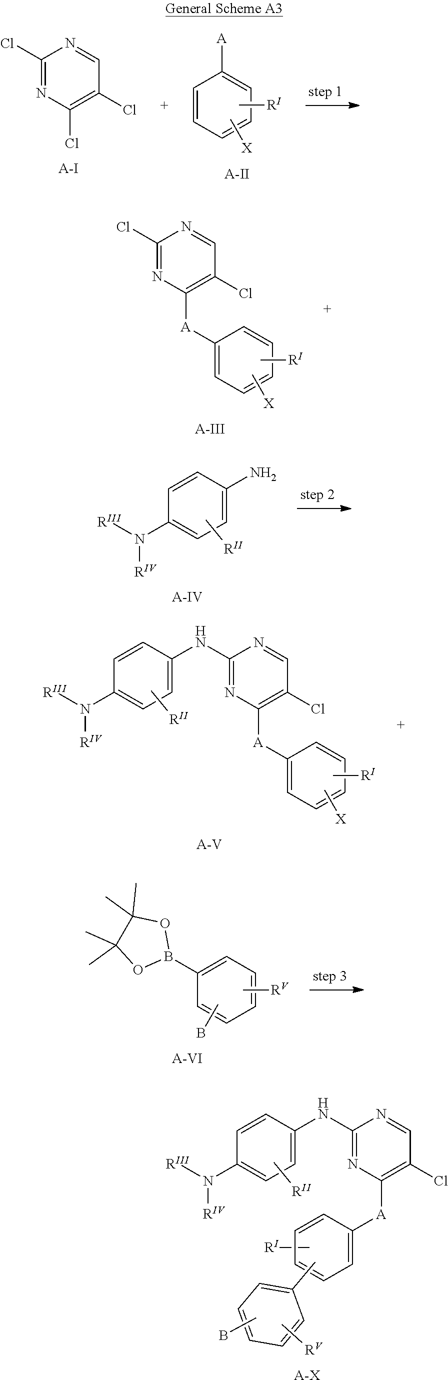

General Scheme A3 provides an alternative synthesis of 2-arylaminopyrimidines. In Step 1, 2,4,5-trihalopyridimine A-I can readily undergo a regioselective nucleophilic aromatic substitution with bromo- or iodo-containing nucleophile A-II and an appropriate base to provide aminophenylpyrimidine A-III (A=N), or phenoxypyrimidine III (A=O). In Step 2, substituted halopyridimine A-III can undergo a second regioselective nucleophilic aromatic substitution with aniline A-IV to provide 2-arylaminopyrimidine V. In Step 3, 2-arylaminopyrimidine A-V can be reacted with aryl boronic ester or aryl boronic acid VI via a selective Suzuki cross coupling reaction to provide 2-arylaminopyrimidines A-X. In some cases, the aryl boronic ester or ester A-VI can contain an aldehyde “B”. The aldehyde “B” can also be protected with a suitable protecting group such as an acetal with ethylene glycol, prior to Suzuki coupling. In this case, the protected aldehyde “B” can be unmasked with acid such as trifluoroacetic acid or methanesulfonic acid to provide the desired 2-arylaminopyrimidines A-X.

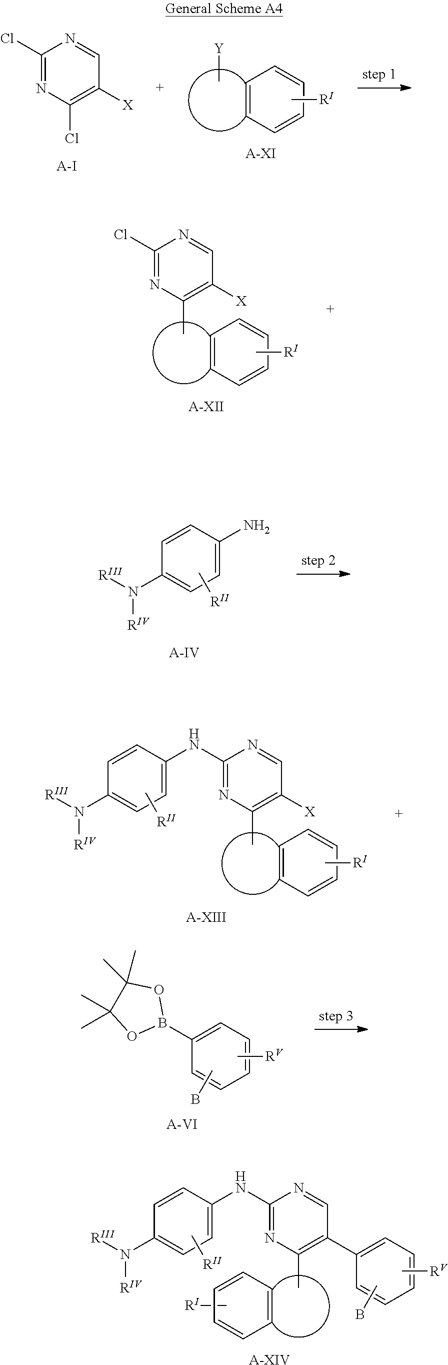

General Scheme A4 provides an alternative synthesis of 2-arylaminopyrimidines. In Scheme A4, “Y” can be a boronic acid or boronic ester. In Step 1, 2,4,5-trihalopyridimine A-I can undergo a regioselective cross coupling reaction with aryl or heteroaryl boronic acid A-XI to provide aryl- or heteroarylpyrimidine A-XII. In Step 2, substituted halopyridimine XIII can undergo a regioselective nucleophilic aromatic substitution with aniline A-IV to provide 2-arylaminopyrimidine A-V. In Step 3, 2-arylaminopyrimidine A-V can be reacted with aryl boronic ester or aryl boronic acid A-VI via Suzuki cross coupling reaction to provide 2-arylaminopyrimidine A-XIV. In some cases, the aryl boronic ester or ester A-VI can contain an aldehyde “B”. The aldehyde “B” can also be protected with a suitable protecting group such as an acetal with ethylene glycol, prior to Suzuki coupling. In this case, the protected aldehyde “B” can be unmasked with acid such as trifluoroacetic acid or methanesulfonic acid to provide the desired 2-arylaminopyrimidines A-XIV.

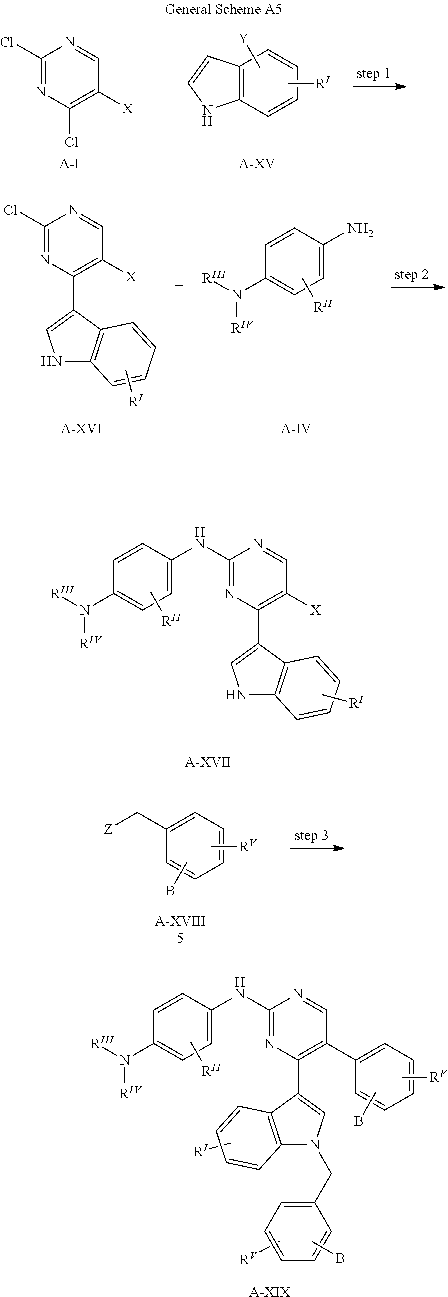

General Scheme A5 provides an alternative synthesis of 2-arylaminopyrimidines. In Scheme A5, “Y” can be a boronic acid, or boronic ester. In Step 1, 2,4,5-trihalopyridimine A-I can undergo a regioselective cross coupling reaction with indolylboronic acid A-XV to provide indolylpyrimidine A-XVI. In Step 2, substituted halopyridimine A-XVI can undergo a regioselective nucleophilic aromatic substitution with aniline A-IV to provide 2-arylaminopyrimidine A-V. In Step 3, halopyridimine A-XVII can undergo a Mitsunobu reaction with benzyl alcohol A-XVIII (Z═OH), or a nucleophilic substitution reaction with benzyl halide A-XVIII (Z═Br, or Cl) to provide substituted indolylpyrimidine A-XIX. In some cases, the benzyl alcohol or halide A-XVIII can contain an aldehyde “B”. The aldehyde “B” can also be protected with a suitable protecting group such as an acetal with ethylene glycol, prior to Suzuki coupling. In this case, the protected aldehyde “B” can be unmasked with acid such as trifluoroacetic acid or methanesulfonic acid to provide the desired 2-arylaminopyrimidines A-XIX.

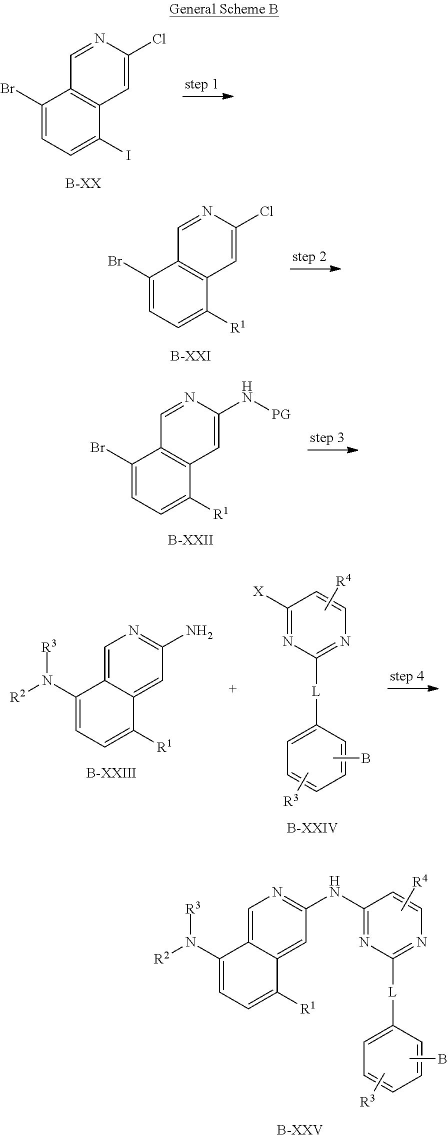

Scheme B provides a general synthesis of 3-aminoisoquinolines. In Step 1, 3,5,8-trihaloisoquinoline B-XX can undergo a regioselective cross coupling reaction with alkyl- or alkenyl-boronic ester or boronic acid in presence of a suitable transition metal catalyst to form the desired 5-alkyl, or 5-alkenyl substituted dihaloisoquinoline B-XXI. Next, substituted 3-chloroisoquinoline B-XXI can readily react in a regioselective nucleophilic aromatic substitution with a protected amine to provide the desired protected 3-aminoisoquinoline B-XXII. In step 3, substituted 8-bromoisoquinoline B-XXII can undergo a Buchwald-type amination with a corresponding amine and suitable transition metal catalyst. Upon deprotection of the amine, the desired diaminoisoquinoline B-XXIII can be obtained. Finally, in step 4, 3-aminoisoquionline B-XXIII undergo a second Buchwald-type amination with a corresponding amine fragment B—XXIV and suitable transition metal catalyst to provide the desired 3-aminoisoquionline B-XXV. In some cases, the amine fragment B—XXIV may contain an aldehyde “B”. The aldehyde of can also be protected with a suitable protecting group such as an acetal with ethylene glycol, prior to reaction. In this case, the protected aldehyde can be unmasked with acid such as trifluoroacetic acid or methanesulfonic acid in the final step to provide the desired 3-aminoisoquionline B-XXV. In Scheme B, “L” corresponds to a linker separating two aromatic rings. The Linker “L” can be an aryl- or heteroaryl- ring or an internal alkyne, which can be connected via standard cross coupling conditions such as Suzuki, Buchwald, or Sonogashira cross coupling reactions. The Linker “L” can also be an aliphatic or heteroatom containing linker of various atom lengths, which can be connected via standard nucleophilic substitution or nucleophilic aromatic substitution.

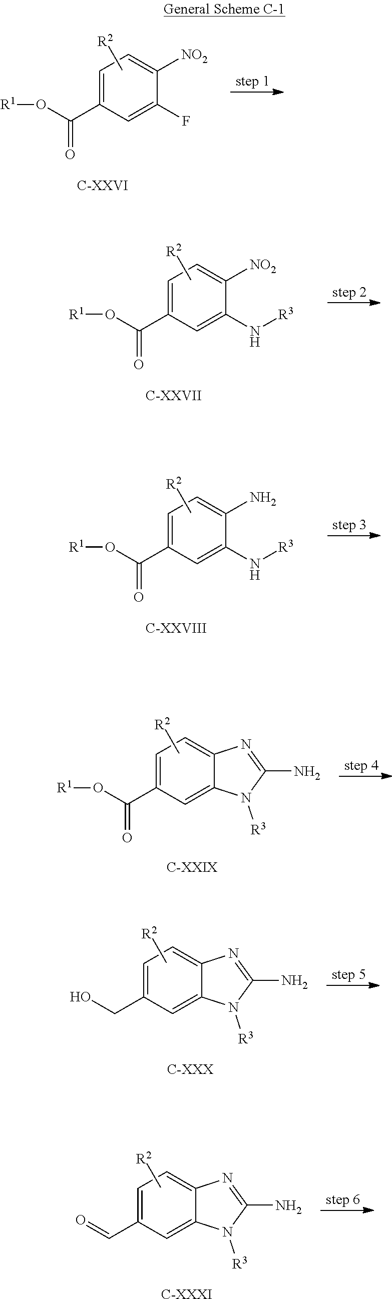

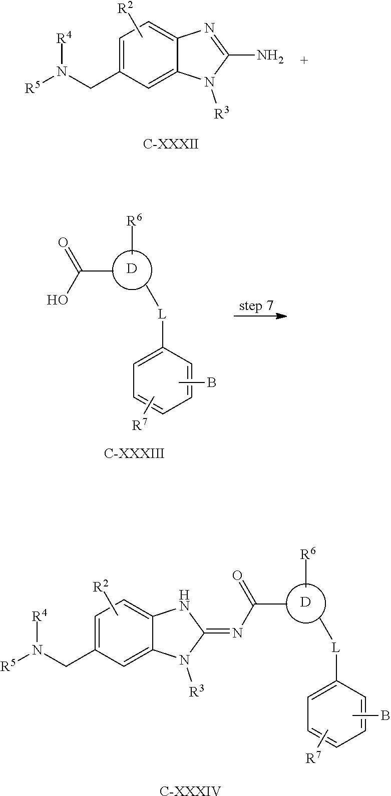

Scheme C-1 provides a general synthesis of Aminobenzimidazoles. In Step 1, aryl fluoride C—XXVI can undergo a nucleophilic aromatic substitution with a corresponding amine to generate the desired aniline C-XXVII. In step two, Nitroaniline C-XXVII can be reduced to the corresponding free aniline C-XXVIII via standard reductive conditions such as catalytic hydrogenation or in acidic media in presence of iron. In Step 3, aniline C-XXVIII can be heated with cyanogen bromide to form the desired amino-benzimidazole C—XXIX upon ring closure. In Step 4, the ester of amino-benzimidazole C—XXIX, can be reduced to the alcohol with a suitable hydride, and oxidized to the corresponding aldehyde C—XXXI with manganese (IV) oxide, or Dess-Martin periodinane. Alternatively, Ester C—XXIX can also be hydrolyzed to provide the carboxylic acid, which can be subject to further derivatization to provide amides via standard peptide coupling reactions. In Step 6, Aldehyde C—XXXI can readily undergo reductive amination with a suitable amine to provide the desired aminobenzimidazole C—XXXII. Aminobenzimidazole C—XXXII can next to subjected to standard peptide coupling conditions with carboxylic acid C—XXXIII in step 7 to provide the desired aminobenzimidazoles C—XXXIV. In Scheme C-1, “D” represents an optionally substituted aryl or heteroaryl ring. “L” corresponds to a linker separating two aromatic rings. The Linker “L” can be an aryl- or heteroaryl- ring or an internal alkyne, which can be connected via standard cross coupling conditions such as Suzuki, Buchwald, or Sonogashira cross coupling reactions.

The Linker “L” can also be an aliphatic or heteroatom containing linker of various atom lengths, which can be connected via standard nucleophilic substitution or nucleophilic aromatic substitution.

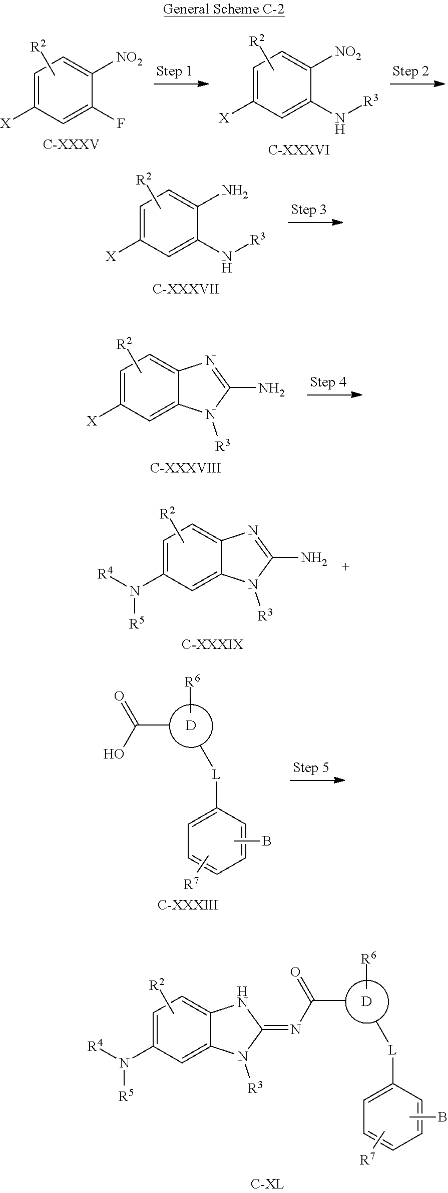

Scheme C-2 provides a general synthesis of Aminobenzimidazoles. In Step 1, aryl fluoride C—XXXV can undergo a nucleophilic aromatic substitution with a corresponding amine to generate the desired aniline C-XXXVI. In Step 2, Nitroaniline C-XXXVI can be reduced to the corresponding free aniline C-XXXVII via standard reductive conditions such as catalytic hydrogenation or in acidic media in presence of iron. In Step 3, aniline C-XXXVII can be heated with cyanogen bromide to form the desired amino-benzimidazole C—XXXVIII upon ring closure. In Step 4, Halo-aminobenzimidazole C—XXXVIII undergo a Buchwald-type amination with a corresponding amine and suitable transition metal catalyst to provide the desired di-aminobenzimidazole C—XXXIX. Aminobenzimidazole C—XXXIX can next to subjected to standard peptide coupling conditions with carboxylic coupling conditions with carboxylic acid C—XXXIII in step 5 to provide the desired aminobenzimidazoles C-XL. In Scheme C-2, “D” represents an optionally substituted aryl or heteroaryl ring. “L” Corresponds to a linker separating two aromatic rings. The Linker “L” can be an aryl- or heteroaryl- ring or an internal alkyne, which can be connected via standard cross coupling conditions such as Suzuki, Buchwald, or Sonogashira cross coupling reactions. The Linker “L” can also be an aliphatic or heteroatom containing linker of various atom lengths, which can be connected via standard nucleophilic substitution or nucleophilic aromatic substitution.

### Example 2: Representative Compound Syntheses Following General Schemes A1-A5

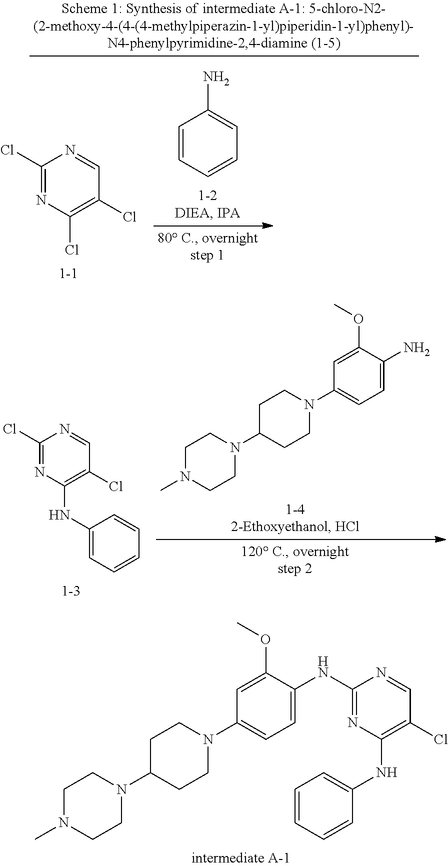

### Step 1: Synthesis of 2,5-dichloro-N-phenylpyrimidin-4-amine

To a solution of 2,4,5-trichloropyrimidine (20 g, 109.039 mmol) in 2-Propanol (200 mL) was added aniline (11.17 g, 119.943 mmol), DIEA (21.14 g, 163.559 mmol). The resulting mixture was maintained under nitrogen and stirred at 80° C. for overnight. LCMS showed the desired product was generated. The reaction was concentrated. The residue obtained was purified by silica gel chromatography (0-20% EtOAc/petroleum ether) to afford 2,5-dichloro-N-phenylpyrimidin-4-amine as a yellow solid (25 g, 95.50% yield). Mass calculated for C₁₀H₇C₁₂N₃: 240.09, found: 241.12 [M+H]⁺.

### Step 2: Synthesis of 5-chloro-N₂-(2-methoxy-4-(4-(4-methylpiperazin-1-yl)piperidin-1-yl)phenyl)-N₄-phenylpyrimidine-2,4-diamine (1-5)

To a solution of 2,5-dichloro-N-phenylpyrimidin-4-amine (4 g, 16.660 mmol) in 2-ethoxyethan-1-ol (40 mL) was added 2-methoxy-4-[4-(4-methylpiperazin-1-yl)piperidin-1-yl]aniline (5.07 g, 16.660 mmol), HCl(g) in MeOH (12 mL). The resulting mixture was maintained under nitrogen and stirred at 120° C. overnight. LCMS showed the desired product was generated. After cooling down to rt, the reaction was quenched with H₂O (100 mL). The pH value of the aqueous phase was adjusted to 7-8 used NaHCO₃ solution. The resulting mixture was extracted with EtOAc (3×100 mL). The organic layers were combined, dried over Na₂SO₄, filtered and concentrated. It was afforded the 5-chloro-N₂-{2-methoxy-4-[4-(4-methylpiperazin-1-yl)piperidin-1-yl]phenyl}-N₄-phenylpyrimidine-2,4-diamine as a yellow solid (5.5 g, 64.98% yield). Mass calculated for C₂₇H₃₄ClN₇O: 508.07, found: 509.15 [M+H]⁺.

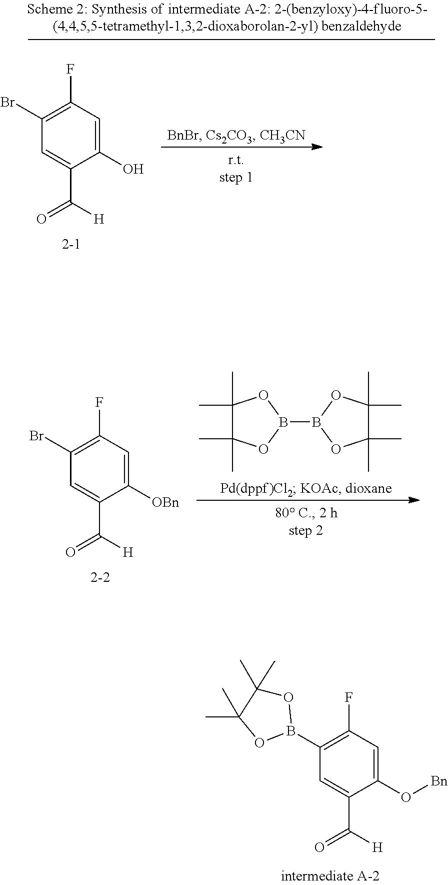

### Step 1: 2-(benzyloxy)-S-bromo-4-fluorobenzaldehyde

To a solution of 5-bromo-4-fluoro-2-hydroxybenzaldehyde (500 mg, 2.283 mmol) in CH₃CN (20 mL) was added BnBr (585.72 mg, 3.425 mmol), Cs₂CO₃ (2.23 g, 6.849 mmol). The resulting mixture was stirred at rt for overnight. LCMS showed the desired product was generated. The reaction was quenched with H₂O (50 mL). The resulting mixture was extracted with EtOAc (3×50 mL). The organic layers were combined, dried over Na₂SO₄, filtered and concentrated. The residue obtained was purified by silica gel chromatography (0-15% EtOAc/petroleum ether) to afford 2-(benzyloxy)-S-bromo-4-fluorobenzaldehyde as a yellow solid (630 mg, 89.26% yield). MS (ESI) calculated for C₁₄H₁₀BrFO_{2-: 309.15} m/z, found: 310.20 [M+H]⁺.

### Step 2: Synthesis of 2-(benzyloxy)-4-fluoro-S-(4,4,5,5-tetramethyl-1,3,2-dioxaborolan-2-yl)benzaldehyde

To a solution of 2-(benzyloxy)-S-bromo-4-fluorobenzaldehyde (500 mg, 1.617 mmol, 1 equiv) and bis(pinacolato)diboron (821.46 mg, 3.234 mmol, 2 equiv) in 1,4-dioxane (10 mL) were added KOAc (317.47 mg, 3.234 mmol, 2 equiv) and Pd(dppf)Cl₂ (118.35 mg, 0.162 mmol, 0.1 equiv). After stirring for overnight at 80° C. under a nitrogen atmosphere, the resulting mixture was concentrated under reduced pressure. The residue was purified by reverse-phase flash chromatography with the following conditions: Column, Cat No: SO230120-2, C18, 120 g, 20˜45 μm, 100 Å; mobile phase, CH₃CN:H₂O (0.05% NH₄HCO₃) -20% increased to CH₃CN:H₂O (0.05% NH₄HCO₃)=70% in 40 min, hold CH₃CN:H₂O (0.05% NH₄HCO₃)=70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 2-(benzyloxy)-4-fluoro-S-(4,4,5,5-tetramethyl-1,3,2-dioxaborolan-2-yl)benzaldehyde (380 mg, 65.96%) as a yellow solid. MS (ESI) calculated for C₂₀H₂₂BFO₄: 356.16 m/z, found: 357.15 [M+H]⁺.

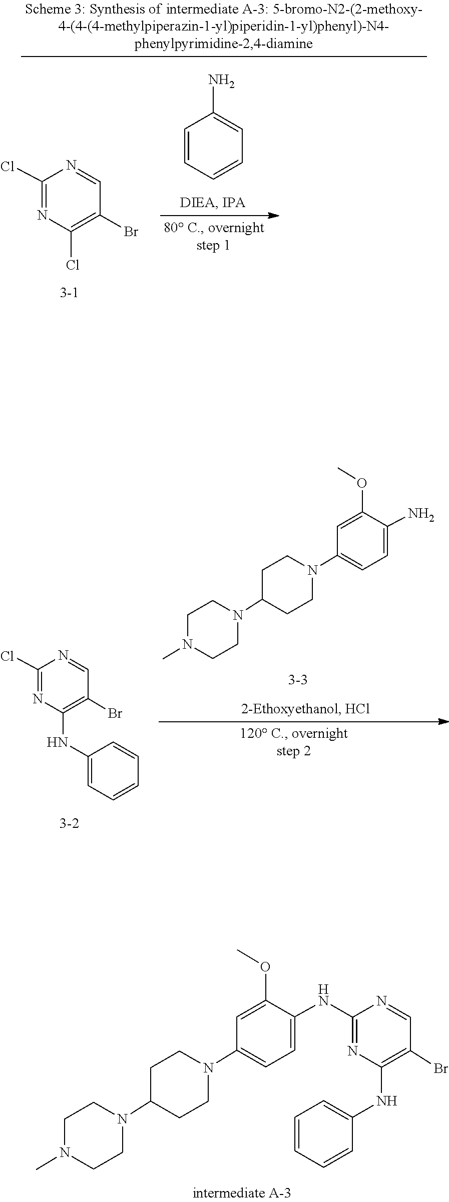 [SMILES: Clc1ncc(Br)c(Cl)n1 ; Nc1ccccc1 ; Clc1ncc(Br)c(Nc2ccccc2)n1 ; COc1cc(N2CCC(N3CCN(C)CC3)CC2)ccc1N ; COc1cc(N2CCC(N3CCN(C)CC3)CC2)ccc1Nc1ncc(Br)c(Nc2ccccc2)n1]

### Step 1: Synthesis of 5-bromo-2-chloro-N-phenylpyrimidin-4-amine

To a colorless solution of 5-bromo-2,4-dichloropyrimidine (4 g, 17.554 mmol, 1 equiv) in i-PrOH (30 mL), DIEA (3.40 g, 26.331 mmol, 1.5 equiv) and aniline (1.80 g, 19.309 mmol, 1.1 equiv) were added at 0° C. giving a light yellow solution. The obtained solution was stirred overnight at 80° C. giving a light yellow solution. The reaction was quenched by the addition of NH₄Cl aq. (5 mL) at 0° C. The resulting mixture was extracted with EA (50 mL×3). The combined organic layers were washed with water (50 mL×3), dried over anhydrous Na₂SO₄. After filtration, the filtrate was concentrated under reduced pressure. The residue was purified by silica gel column (0-6% PE/EA) giving 5-bromo-2-chloro-N-phenylpyrimidin-4-amine (4.27 g, 85.49%) as a light yellow solid. Mass calculated. for C₁₀H₇BrClN₃: 282.95, found: 283.96 [M+H]⁺.

### Step 2: Synthesis of 5-bromo-N2-{2-methoxy-4-[4-(4-methylpiperazin-1-yl)piperidin-1-yl]phenyl}-N4-phenylpyrimidine-2,4-diamine

To a solution of 5-bromo-2-chloro-N-phenylpyrimidin-4-amine (1 g, 3.514 mmol, 1 equiv) in 2-ethoxyethan-1-ol (20 mL) was added 2-methoxy-4-[4-(4-methylpiperazin-1-yl)piperidin-1-yl]aniline (1.07 g, 3.514 mmol, 1 equiv), HCl (4 M in MeOH, 2.2 mL). The resulting mixture was maintained under nitrogen and stirred at 120° C. for overnight. After cooling down to rt, the reaction was quenched with H₂O (20 mL). The pH value of the aqueous phase was adjusted to 7-8 used NaHCO₃ solution. The resulting mixture was extracted with EtOAc (3×100 mL). The organic layers were combined, dried over Na₂SO₄, filtered and concentrated. It was afforded the 5-bromo-N2-{2-methoxy-4-[4-(4-methylpiperazin-1-yl)piperidin-1-yl]phenyl}-N4-phenylpyrimidine-2,4-diamine as a yellow solid (1.26 g, 64.98% yield). Mass calculated for C₂₇H₃₄BrN₇O: 551.20, found: 552.20 [M+H]⁺.

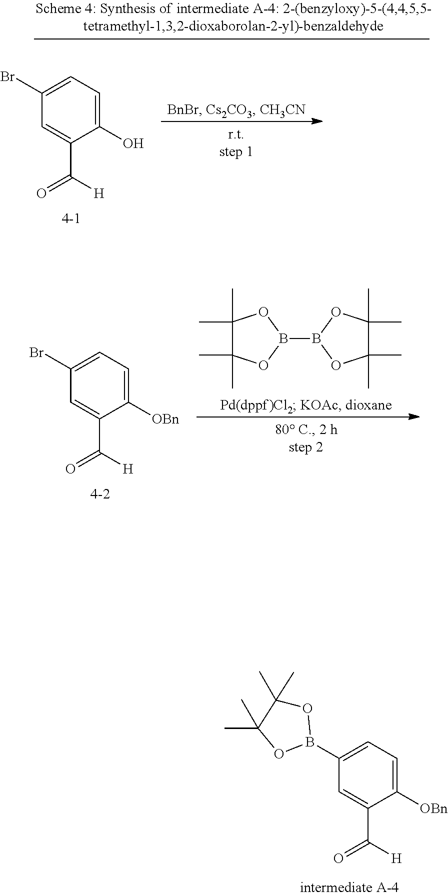

### Step 1: Synthesis of 2-(benzyloxy)-S-bromobenzaldehyde

To a solution of 5-bromo-2-hydroxybenzaldehyde (456.0 mg, 2.283 mmol) in CH₃CN (20 mL) was added BnBr (585.72 mg, 3.425 mmol), Cs₂CO₃ (2.23 g, 6.849 mmol). The resulting mixture was stirred at rt for overnight. LCMS showed the desired product was generated. The reaction was quenched with H₂O (50 mL). The resulting mixture was extracted with EtOAc (3×50 mL). The organic layers were combined, dried over Na₂SO₄, filtered and concentrated. The residue obtained was purified by silica gel chromatography (0-15% EtOAc/petroleum ether) to afford 2-(benzyloxy)-S-bromobenzaldehyde as a white solid (500 mg, 75.52% yield). MS (ESI) calculated for C₁₄H₁₁BrO_{2-: 289.99} m/z, found: 290.10 [M+H]⁺.

### Step 2: Synthesis of 2-(benzyloxy)-5-(4,4,5,5-tetramethyl-1,3,2-dioxaborolan-2-yl)benzaldehyde

To a solution of 2-(benzyloxy)-S-bromobenzaldehyde (469 mg, 1.617 mmol, 1 equiv) and bis(pinacolato)diboron (821.46 mg, 3.234 mmol, 2 equiv) in 1,4-dioxane (10 mL) were added KOAc (317.47 mg, 3.234 mmol, 2 equiv) and Pd(dppf)Cl₂ (118.35 mg, 0.162 mmol, 0.1 equiv). After stirring for overnight at 80° C. under a nitrogen atmosphere, the resulting mixture was concentrated under reduced pressure. The residue was purified by reverse-phase flash chromatography with the following conditions: Column, Cat No: SO230120˜2, C18, 120 g, 20˜45 μm, 100 Å; mobile phase, CH₃CN:H₂O (0.05% NH₄HCO₃)=20% increased to CH₃CN:H₂O (0.05% NH₄HCO₃)=70% in 40 min, hold CH₃CN:H₂O (0.05% NH₄HCO₃) -70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 2-(benzyloxy)-S-(4,4,5,5-tetramethyl-1,3,2-dioxaborolan-2-yl)benzaldehyde (350 mg, 64.00% yield) as a yellow solid. MS (ESI) calculated for C₂₀H₂₃BO₄: 338.17 m/z, found: 339.15 [M+H]⁺.

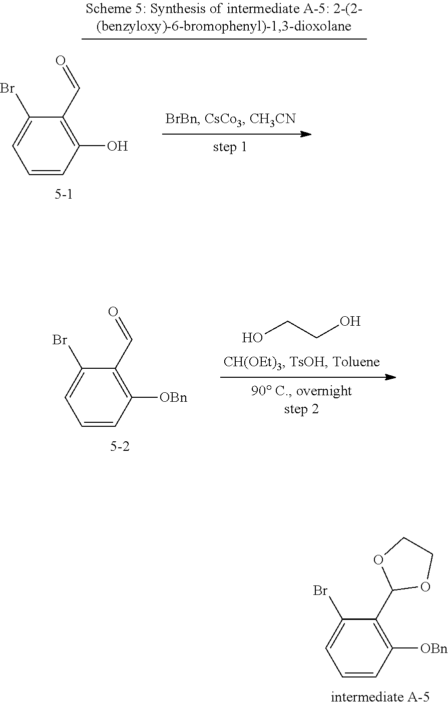

### Step 1: Synthesis of 2-(benzyloxy)-6-bromobenzaldehyde

To a yellow solution of 2-bromo-6-hydroxybenzaldehyde (10 g, 49.747 mmol, 1 equiv) in CH₃CN (450 mL), Cs₂CO₃ (32.42 g, 99.494 mmol, 2 equiv) was added, the suspension was stirred for 10 min giving a yellow suspension. To this suspension, BnBr (12.76 g, 74.620 mmol, 1.5 equiv) was added at rt giving a yellow suspension. The resulting suspension was stirred at rt overnight. The reaction was quenched by the addition of NH₄Cl aq at 0° C. The resulting mixture was extracted with EA (400 mL×3). The combined organic layers were washed with water (500 mL×3), dried over anhydrous Na₂SO₄. After filtration, the filtrate was concentrated under reduced pressure. The residue was purified by silica gel column (0-9% PE/EA) giving 2-(benzyloxy)-6-bromobenzaldehyde (10.1 g, 69.73% yield) as a light yellow solid. Mass calculated For C₁₄H₁₁BrO_{2-: 289.99}, found: 290.95 [M+H]⁺.

### Step 2: Synthesis of 2-[2-(benzyloxy)-6-bromophenyl]-1,3-dioxolane

To a colorless solution of 2-(benzyloxy)-6-bromobenzaldehyde (5 g, 17.174 mmol, 1 equiv) in toluene (70 mL) was added TsOH (0.30 g, 1.717 mmol, 0.1 equiv), ethylene glycol (5.33 g, 85.870 mmol, 5 equiv) and triethyl orthoformate (7.64 g, 51.522 mmol, 3 equiv) at rt. The obtained colorless solution was stirred at rt for 10 min giving a colorless solution. The resulting solution was stirred overnight at 90° C. After cooled down to rt, the reaction was quenched by the addition of NH₄Cl aq (50 mL) at 0° C. The resulting mixture was extracted with EA (50 mL×3). The combined organic layers were washed with water (100 mL×3), dried over anhydrous Na₂SO₄. After filtration, the filtrate was concentrated under reduced pressure.

The residue was purified by silica gel column (0-7% PE/EA) giving 2-[2-(benzyloxy)-6-bromophenyl]-1,3-dioxolane (4.97 g, 86.34% yield) as a light yellow solid. Mass calculated for C₁₆H₁₅BrO₃: 334.02, found: 335.05 [M+H]⁺.

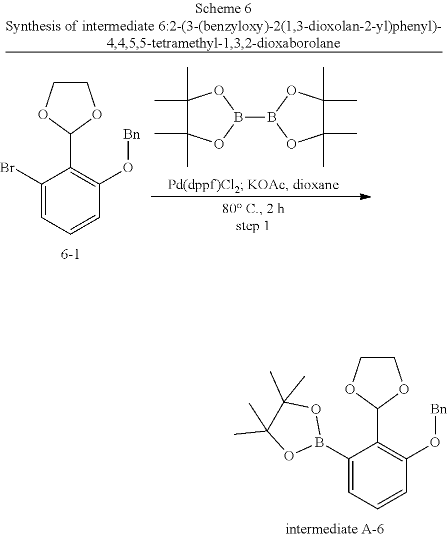

### Step 1: Synthesis of 2-(3-(benzyloxy)-2-(1,3-dioxolan-2-yl)phenyl)-4,4,5,5-tetramethyl-1,3,2-dioxaborolane

To a solution of 2-(2-(benzyloxy)-6-bromophenyl)-1,3-dioxolane (540 mg, 1.617 mmol, 1 equiv) and bis(pinacolato)diboron (821.46 mg, 3.234 mmol, 2 equiv) in 1,4-dioxane (10 mL) were added KOAc (317.47 mg, 3.234 mmol, 2 equiv) and Pd(dppf)Cl₂ (118.35 mg, 0.162 mmol, 0.1 equiv). After stirring for overnight at 80° C. under a nitrogen atmosphere, the resulting mixture was concentrated under reduced pressure. The residue was purified by reverse-phase flash chromatography with the following conditions: Column, Cat No: SO230120˜2, C18, 120 g, 20˜45 μm, 100 Å; mobile phase, CH₃CN:H₂O (0.05% NH₄HCO₃) -20% increased to CH₃CN:H₂O (0.05% NH₄HCO₃)=70% in 40 min, hold CH₃CN:H₂O (0.05% NH₄HCO₃)=70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 2-(3-(benzyloxy)-2-(1,3-dioxolan-2-yl)phenyl)-4,4,5,5-tetramethyl-1,3,2-dioxaborolane (400 mg, 64.72% yield) as a yellow solid. MS (ESI) calculated for C₂₂H₂₇BO₅: 382.20 m/z, found: 383.15 [M+H]⁺.

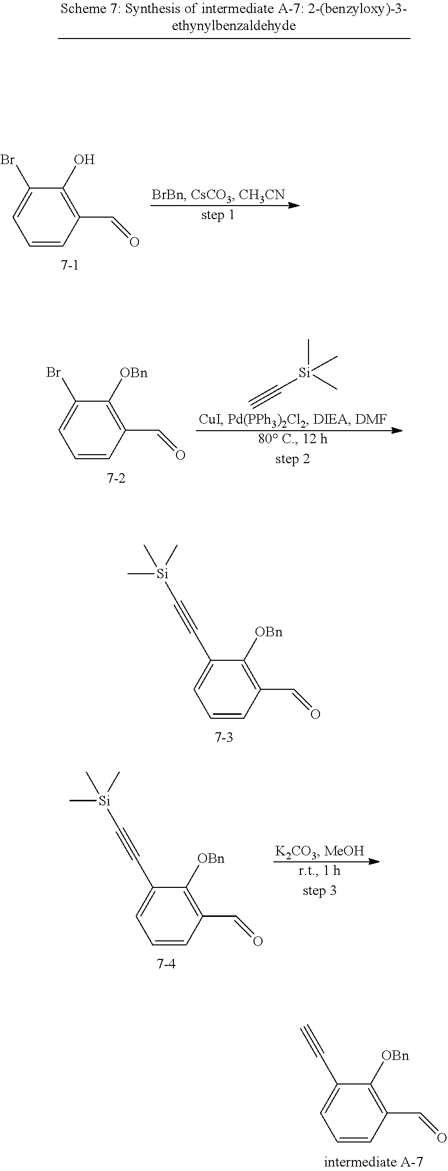

### Step 1: Synthesis of 2-(benzyloxy)-3-bromobenzaldehyde

To a yellow solution of 3-bromo-2-hydroxybenzaldehyde (10 g, 49.747 mmol, 1 equiv) in CH₃CN (450 mL, 8560.885 mmol, 172.09 equiv), Cs₂CO₃ (32.42 g, 99.494 mmol, 2 equiv) was added, the suspension was stirred for 10 min giving a yellow suspension. To this suspension, BnBr (12.76 g, 74.620 mmol, 1.5 equiv) was added at rt giving a yellow suspension. The resulting suspension was stirred at rt overnight. The reaction was quenched by the addition of NH₄Cl aq at 0° C. The resulting mixture was extracted with EA (400 mL×3).

The combined organic layers were washed with water (500 mL×3), dried over anhydrous Na₂SO₄. After filtration, the filtrate was concentrated under reduced pressure. The residue was purified by silica gel column (0-9% PE/EA) giving 2-(benzyloxy)-3-bromobenzaldehyde (10.1 g, 69.73% yield) as a light yellow solid. Mass calculated For C₁₄H₁₁BrO_{2-: 289.99}, found: 290.95 [M+H]⁺.

### Step 2: Synthesis of 2-(benzyloxy)-3-[2-(trimethylsilyl)ethynyl]benzaldehyde

To a suspension of 2-(benzyloxy)-3-bromobenzaldehyde (1 g, 3.435 mmol, 1 equiv), CuI (0.03 g, 0.172 mmol, 0.05 equiv) and Pd(PPh₃)₂C₁₂ (0.12 g, 0.172 mmol, 0.05 equiv) in DMF (10 mL) was added DIEA (0.89 g, 6.870 mmol, 2 equiv) and trimethylsilylacetylene (1.01 g, 10.305 mmol, 3 equiv) dropwise at rt under nitrogen atmosphere. The obtained solution was stirred for 16 h at 80° C. giving a dark brown solution. After cooled down to rt, the reaction was quenched by the addition of NH₄Cl aq. (10 mL). The resulting mixture was extracted with EA (3×20 mL). The combined organic layers were washed with water (3×20 mL), dried over anhydrous Na₂SO₄. After filtration, the filtrate was concentrated under reduced pressure. The residue was purified by reverse flash chromatography with the following conditions: column, C18 silica gel; mobile phase, ACN in water, 10% to 65% gradient in 60 min; detector, UV 254 nm giving 2-(benzyloxy)-3-[2-(trimethylsilyl)ethynyl]benzaldehyde (600 mg, 56.63% yield) as a light brown oil. Mass calculated for C₁₉H₂₀O₂Si: 308.12, found: 309.15 [M+H]⁺.

### Step 3: Synthesis of 2-(benzyloxy)-3-ethynylbenzaldehyde

To a light yellow solution of 2-(benzyloxy)-3-[2-(trimethylsilyl)ethynyl]benzaldehyde (400 mg, 1.297 mmol, 1 equiv) in MeOH (10 mL), K₂CO₃ (537.67 mg, 3.891 mmol, 3 equiv) was added at 0° C. The reaction was quenched by the addition of NH₄Cl aq. (10 mL). The resulting mixture was extracted with EA (20 mL×3). The combined organic layers were washed with water (20 mL×3), dried over anhydrous Na₂SO₄. After filtration, the filtrate was concentrated under reduced pressure giving 2-(benzyloxy)-3-ethynylbenzaldehyde (300 mg, 97.91% yield) as a light brown oil. Mass calculated. for C₁₆H₁₂O₂: 236.08, found: 237.15 [M+H]⁺.

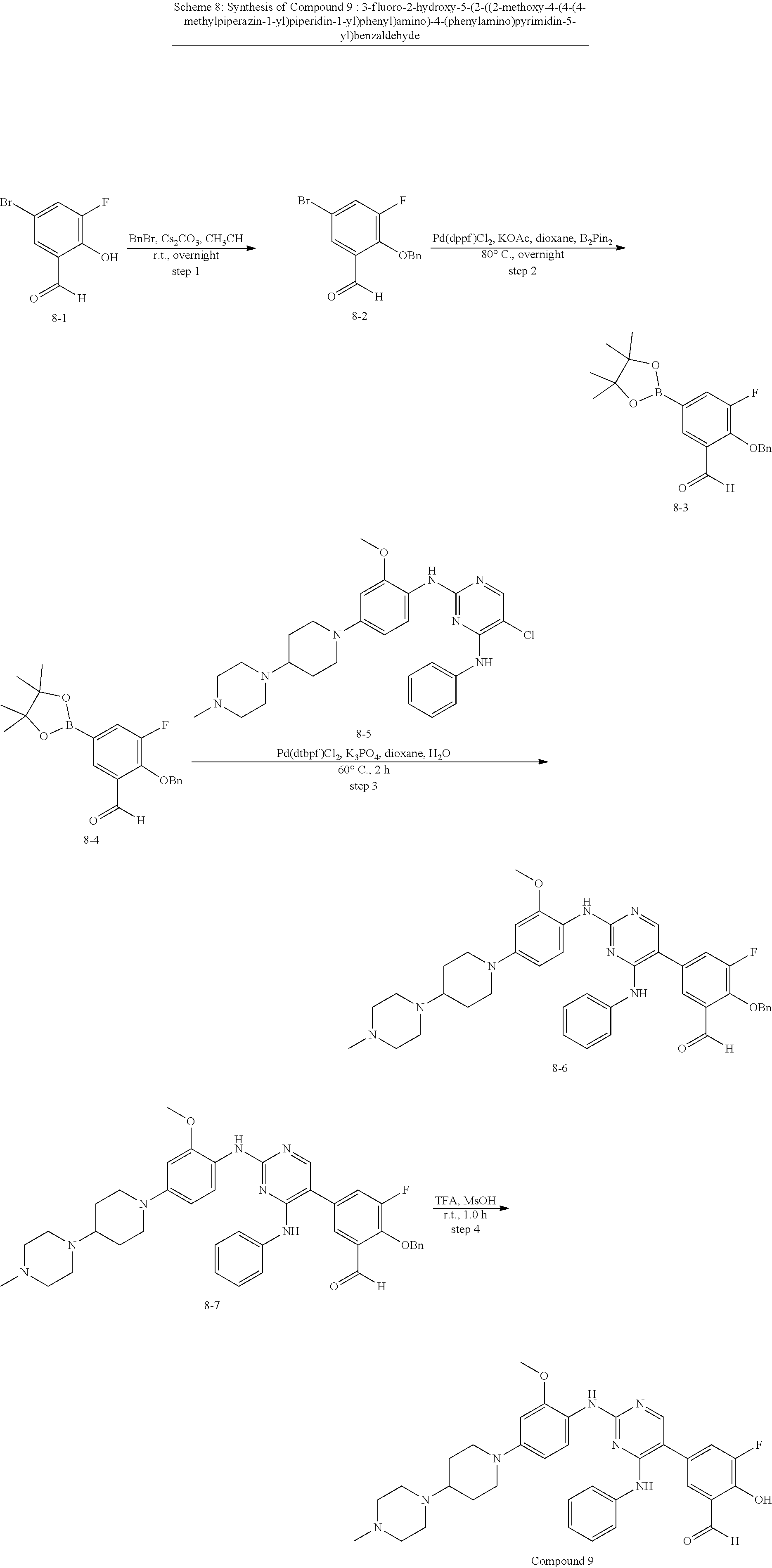

### Step 1: Synthesis of 2-(benzyloxy)-S-bromo-3-fluorobenzaldehyde

5-bromo-3-fluoro-2-hydroxybenzaldehyde (1 g, 4.566 mmol, 1 equiv), CH₃CN (10 mL) were added to a 25 mL round-bottom flask and stirred until homogeneous. Cs₂CO₃ (3719.24 mg, 11.415 mmol, 2.5 equiv) and BnBr (937.15 mg, 5.479 mmol, 1.2 equiv) were added to the reaction flask at rt. The resulting mixture was stirred at rt for overnight. The reaction mixture was then treated with H₂O (30 mL), dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The oil was then subjected to silica gel chromatography (0-10% EtOAc/pet ether) to give 2-(benzyloxy)-S-bromo-3-fluorobenzaldehyde (1.3 g, 92.10%) as a white solid. ¹H NMR (400 MHz, DMSO-d₆) δ (ppm): 10.03 (s, 1H), 8.02 (dd, J₁=2.4 Hz, J_{2-=10.8} Hz, 1H), 7.59-7.60 (m, 1H), 7.30-7.46 (m, 5H), 5.16 (s, 2H).

### Step 2: Synthesis of 2-(benzyloxy)-3-fluoro-S-(4,4,5,5-tetramethyl-1,3,2-dioxaborolan-2-yl) benzaldehyde

2-(benzyloxy)-S-bromo-3-fluorobenzaldehyde (1 g, 3.235 mmol, 1 equiv), dioxane (10 mL) were added to a 50-mL round-bottom flask and stirred until homogeneous. Bis(pinacolato)diboron (903.60 mg, 3.558 mmol, 1.1 equiv), Pd(dppf)Cl₂ (236.70 mg, 0.324 mmol, 0.1 equiv) and KOAc (952.42 mg, 9.705 mmol, 3 equiv) were added to the reaction flask at rt. The resulting mixture was stirred at 80° C. for overnight under N₂. The reaction mixture was then treated with H₂O (30 mL), dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The oil was then subjected to silica gel chromatography (0-10% EtOAc/pet ether) to give 2-(benzyloxy)-3-fluoro-S-(4,4,5,5-tetramethyl-1,3,2-dioxaborolan-2-yl)benzaldehyde (900 mg, 78.11%) as a colorless oil. MS (ESI) calculated for C₂₀H22BFO₄, 356.16 m/z, found 357.16 [M+H]⁺.

### Step 3: Synthesis of 2-(benzyloxy)-3-fluoro-S-[2-({2-methoxy-4-[4-(4-methylpiperazin-1-yl)piperidin-1-yl]phenyl}amino)-4-(phenylamino)pyrimidin-S-yl]benzaldehyde

5-chloro-N2-{2-methoxy-4-[4-(4-methylpiperazin-1-yl)piperidin-1-yl]phenyl}-N4-phenylpyrimidine-2,4-diamine (750.00 mg, 1.476 mmol, 1.5 equiv), dioxane (8 mL) and H₂O (2 mL) were added to a 50-mL round-bottom flask and stirred until homogeneous. 5-chloro-N2-{2-methoxy-4-[4-(4-methylpiperazin-1-yl)piperidin-1-yl]phenyl}-N4-phenylpyrimidine-2,4-diamine (750.00 mg, 1.476 mmol, 1.5 equiv), Pd(dtbpf)Cl₂ (128.28 mg, 0.197 mmol, 0.2 equiv) and K₃PO₄ (522.23 mg, 2.460 mmol, 2.5 equiv) were added to the reaction flask at r.t..

The resulting mixture was stirred at 60° C. for 2 h under N₂. The crude product was purified by reverse-phase flash with the following conditions: Column, Cat No: SO230120˜2, C18, 120 g, 20˜45 μm, 100 Å, Lot: BP0002P2503; mobile phase, CH₃CN:H₂O (0.05% FA)=20% increased to CH₃CN:H₂O (0.05% FA)=70% in 40 min, hold CH₃CN:H₂O (0.05% FA)=70% in 10 min, up to CH₃CN:H₂O (0.05% FA)=95% in 2 min, hold CH₃CN:H₂O (0.05% FA)=95% in 10 min; Detector, UV 220 nm & 254 nm. After lyophilization, it was afforded 2-(benzyloxy)-3-fluoro-S-[2-({2-methoxy-4-[4-(4-methylpiperazin-1-yl)piperidin-1-yl]phenyl}amino)-4-(phenylamino)pyrimidin-S-yl]benzaldehyde (200 mg, 28.96%) as a brown solid. MS (ESI) calcd. for C₄₁H₄₄FN₇O₃, 701.35 m/z, found 702.30 [M+H]⁺.

### Step 4: Synthesis of 3-fluoro-2-hydroxy-S-(2-((2-methoxy-4-(4-(4-methylpiperazin-1-yl)piperidin-1-yl)phenyl)amino)-4-(phenylamino)pyrimidin-S-yl)benzaldehyde

2-(benzyloxy)-3-fluoro-S-[2-({2-methoxy-4-[4-(4-methylpiperazin-1-yl)piperidin-1-yl]phenyl}amino)-4-(phenylamino)pyrimidin-S-yl]benzaldehyde (90 mg, 0.128 mmol, 1 equiv), TFA (2 mL) were added to a 50-mL round-bottom flask and stirred until homogeneous. Methanesulfonic acid (0.4 mL) was added to the reaction flask at r.t.. The resulting mixture was stirred at rt for 1 h. The residue was diluted with water, then adjusted to pH 6-7 with NaHCO₃, dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The crude product was then purified by Prep-HPLC with the following conditions: Column: XBridge Shield RP18 OBD Column, 19*250 mm, 10 μm; Mobile Phase A: Water(0.1% FA), Mobile Phase B: ACN; Flow rate: 25 mL/min; Gradient: 13% B to 34% B in 7 min, 34% B; Wave Length: 254 nm; RT1(min): 4.88; Number Of Runs. After lyophilization, it was afforded the 3-fluoro-2-hydroxy-5-(2-((2-methoxy-4-(4-(4-methylpiperazin-1-yl)piperidin-1-yl)phenyl)amino)-4-(phenylamino)pyrimidin-S-yl)benzaldehyde (3.4 mg, 3.63%) as a light yellow solid. MS (ESI) calcd. for C₃₄H₃₈FN₇O₃, 611.30 m/z, found 612.35 [M+H]⁺. ¹H NMR (300 MHz, DMSO-d₆) δ (ppm): 11.24 (s, 1H), 10.35 (s, 1H), 9.15 (s, 1H), 7.86 (s, 1H), 7.65-7.75 (m, 1H), 7.26-7.61 (m, 6H), 7.12-7.17 (m, 1H), 6.62-6.73 (m, 1H), 6.43-6.46 (m, 1H), 3.77-3.84 (m, 5H), 2.90-2.92 (m, 2H), 2.71-2.72 (m, 2H), 2.67-2.71 (m, 6H), 2.50-2.51 (m, 5H), 1.86-1.96 (m, 2H), 1.49-1.64 (m, 2H). ¹⁹F NMR (282 MHz, DMSO-d₆) δ (ppm): -134.18.

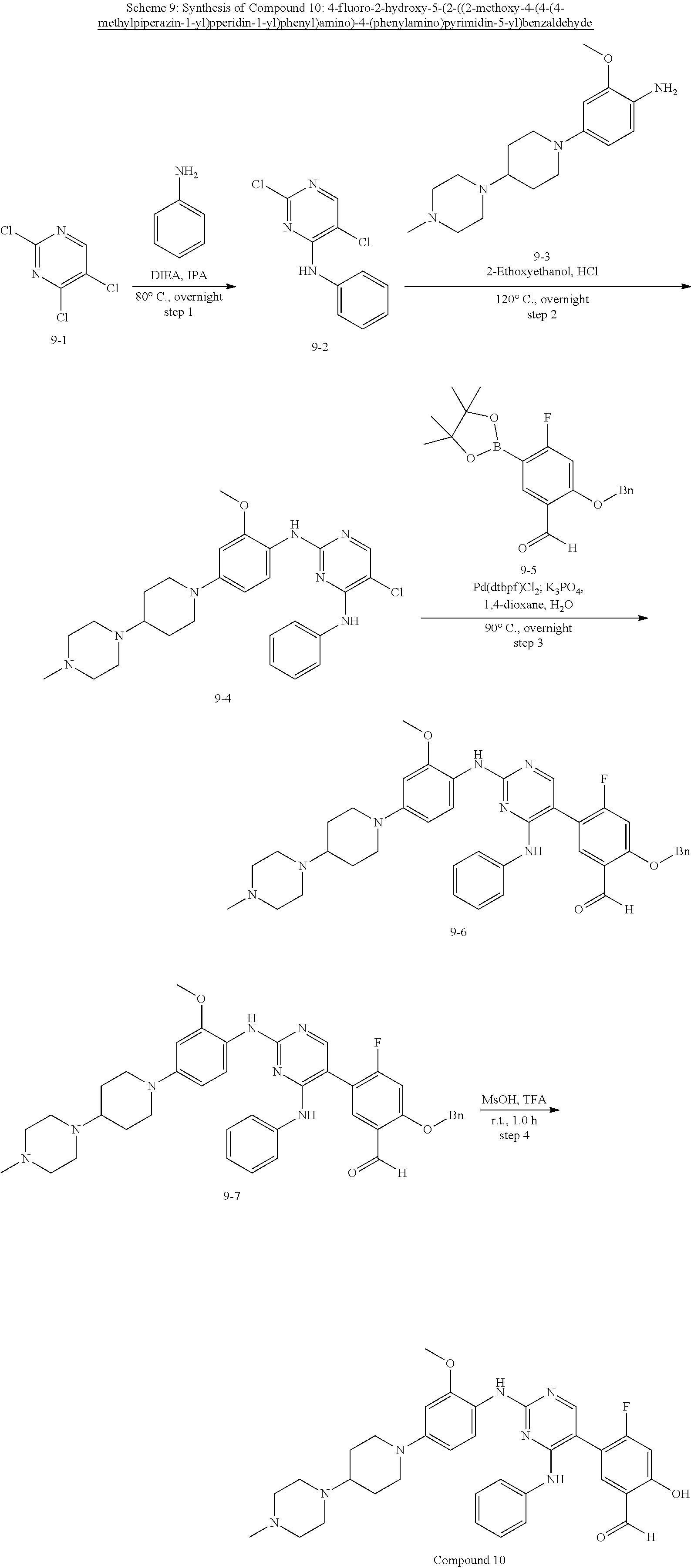

### Step 1: Synthesis of 2,5-dichloro-N-phenylpyrimidin-4-amine

To a solution of 2,4,5-trichloropyrimidine (20 g, 109.039 mmol) in 2-Propanol (200 mL) was added aniline (11.17 g, 119.943 mmol), DIEA (21.14 g, 163.559 mmol). The resulting mixture was maintained under nitrogen and stirred at 80° C. for overnight. LCMS showed the desired product was generated. The reaction was concentrated. The residue obtained was purified by silica gel chromatography (0-20% EtOAc/petroleum ether) to afford 2,5-dichloro-N-phenylpyrimidin-4-amine as a yellow solid (25 g, 95.50% yield). MS (ESI) calculated for C₁₀H₇C₁₂N₃: 240.09 m/z, found: 241.12 [M+H]⁺.

### Step 2: Synthesis of 5-chloro-N2-(2-methoxy-4-(4-(4-methylpiperazin-1-yl)piperidin-1-yl)phenyl)-N4-phenylpyrimidine-2,4-diamine

To a solution of 2,5-dichloro-N-phenylpyrimidin-4-amine (4 g, 16.660 mmol) in 2-ethoxyethan-1-ol (40 mL) was added 2-methoxy-4-[4-(4-methylpiperazin-1-yl)piperidin-1-yl]aniline (5.07 g, 16.660 mmol), HCl(g) in MeOH (12 mL). The resulting mixture was maintained under nitrogen and stirred at 120° C. for overnight. LCMS showed the desired product was generated. After cooling down to rt, the reaction was quenched with H₂O (100 mL). The pH value of the aqueous phase was adjusted to 7-8 using a NaHCO₃ solution. The resulting mixture was extracted with EtOAc (3×100 mL). The organic layers were combined, dried over Na₂SO₄, filtered and concentrated. It was afforded the 5-chloro-N2-{2-methoxy-4-[4-(4-methylpiperazin-1-yl)piperidin-1-yl]phenyl}-N4-phenylpyrimidine-2,4-diamine as a yellow solid (5.5 g, 64.98% yield). MS (ESI) calculated for C₂₇H₃₄ClN₇O: 508.07 m/z, found: 509.15 [M+H]⁺.

### Step 3: Synthesis of 2-(benzyloxy)-4-fluoro-S-(2-((2-methoxy-4-(4-(4-methylpiperazin-1-yl)piperidin-1-yl)phenyl)amino)-4-(phenylamino)pyrimidin-S-yl)benzaldehyde

To a solution of 5-bromo-N2-{2-methoxy-4-[4-(4-methylpiperazin-1-yl)piperidin-1-yl]phenyl}-N4-phenylpyrimidine-2,4-diamine (300 mg, 0.543 mmol) in 1,4-dioxane (8 mL) and H₂O (2 mL) was added 2-(benzyloxy)-4-fluoro-S-(4,4,5,5-tetramethyl-1,3,2-dioxaborolan-2-yl)benzoic acid (303.14 mg, 0.815 mmol), Pd(dtbpf)Cl₂ (35.39 mg, 0.054 mmol), K₃PO4 (288.13 mg, 1.358 mmol). The resulting mixture was maintained under nitrogen and stirred at 90° C. for 3 h. LCMS showed the desired. After cooling down to rt, the reaction was quenched with H₂O (50 mL). The resulting mixture was extracted with EtOAc (3×50 mL). The organic layers were combined, dried over Na₂SO₄, filtered and concentrated. The crude product was purified by reverse-phase flash chromatography with the following conditions: Column, Cat No: SO230120-2, C18, 120 g, 20˜45 μm, 100 Å; mobile phase, CH₃CN:H₂O (0.05% NH₄HCO₃)=20% increased to CH₃CN:H₂O (0.05% NH₄HCO₃)=70% in 40 min, hold CH₃CN:H₂O (0.05% NH₄HCO₃)=70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 2-(benzyloxy)-4-fluoro-S-[2-({2-methoxy-4-[4-(4-methylpiperazin-1-yl)piperidin-1-yl]phenyl}amino)-4-(phenylamino)pyrimidin-S-yl]benzaldehyde as a yellow solid (180 mg, 47.23% yield). MS (ESI) calculated for C₄₁H₄₄FN₇O₃: 701.84 m/z, found: 702.50 [M+H]⁺.

### Step 4: Synthesis of 4-fluoro-2-hydroxy-S-(2-((2-methoxy-4-(4-(4-methylpiperazin-1-yl)piperidin-1-yl)phenyl)amino)-4-(phenylamino)pyrimidin-S-yl)benzaldehyde

To a solution of 2-(benzyloxy)-4-fluoro-S-[2-({2-methoxy-4-[4-(4-methylpiperazin-1-yl)piperidin-1-yl]phenyl}amino)-4-(phenylamino)pyrimidin-S-yl]benzaldehyde (160 mg, 0.228 mmol) in TFA (5 mL) and methanesulfonic acid (1 mL). The resulting mixture was stirred at rt for 1 h. The reaction was quenched with H₂O (10 mL). The pH value of the aqueous phase was adjusted to 7-8 using a NaHCO₃ solution. The resulting mixture was extracted with EtOAc (3×20 mL). The organic layers were combined, dried over Na₂SO₄, filtered and concentrated. The crude product was then purified by Prep-HPLC with the following conditions (1 #-Waters 2767-5): Column, SunFire Prep C18, 5 μm, 19*100 mm; mobile phase, H₂O (0.05% NH₄HCO₃) and CH₃CN (65% CH₃CN up to 75% in 11 min, up to 100% in 1 min, hold 100% in 1 min, down to 65% in 1 min, hold 65% in 1 min); Detector, UV 220&254 nm. After lyophilization, it was afforded the 4-fluoro-2-hydroxy-S-[2-({2-methoxy-4-[4-(4-methylpiperazin-1-yl)piperidin-1-yl]phenyl}amino)-4-(phenylamino)pyrimidin-S-yl]benzaldehyde Trifluoroacetate as a white solid (10.3 mg, 6.03% yield). MS (ESI) calculated for C₃₄H₃₈FN₇O₃, 611.30 m/z, found: 612.30 [M+H]⁺. ¹H NMR (400 MHz, DMSO-d6) δ (ppm): 10.22 (s, 1H), 8.17 (s, 1H), 7.68-7.82 (m, 2H), 7.53-7.65 (m, 2H), 7.21-7.25 (m, 2H), 6.99-7.02 (m, 1H), 6.88-6.91 (m, 1H), 6.62-6.63 (m, 1H), 6.40-6.43 (m, 1H), 3.78 (s, 3H), 3.69-3.72 (m, 2H), 3.56 (s, 3H), 2.65-2.73 (m, 5H), 2.62 (s, 3H), 2.44 (s, 3H), 1.88-1.91 (m, 2H), 1.51-1.59 (s, 2H).

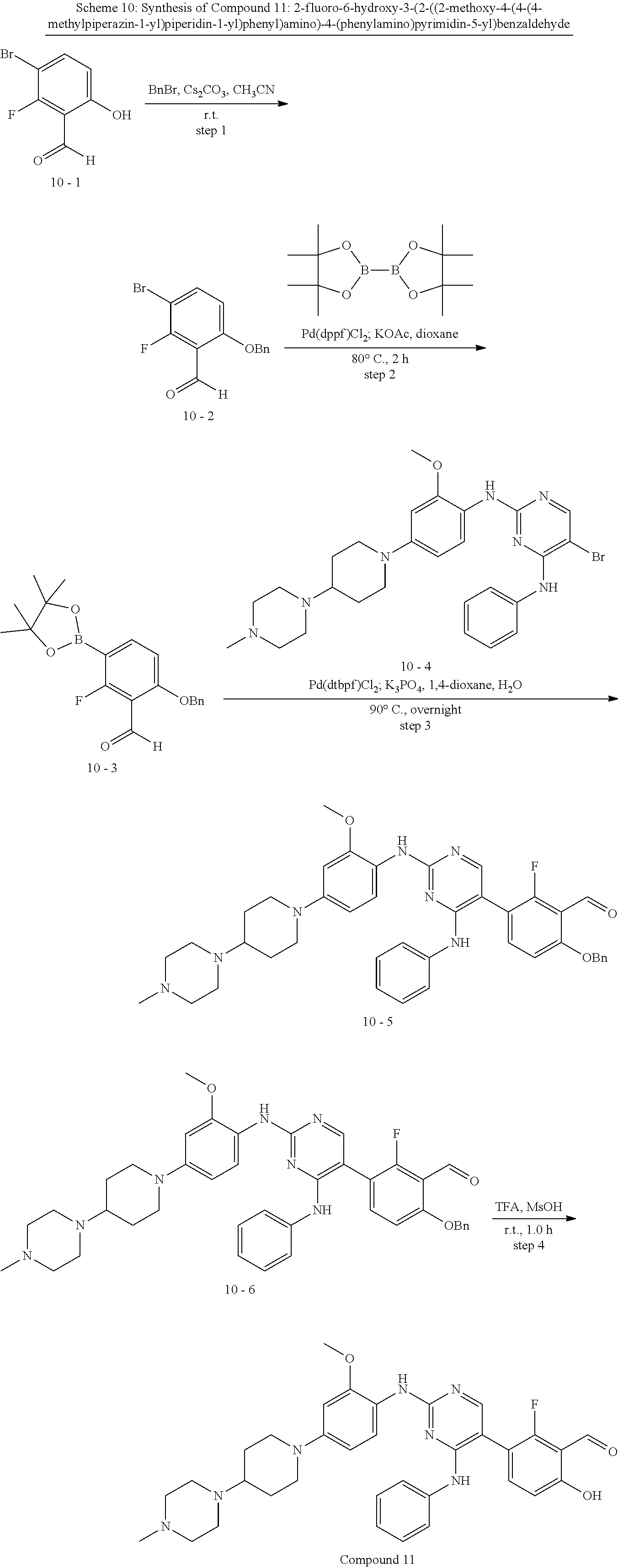

### Step 1: Synthesis of 6-(benzyloxy)-3-bromo-2-fluorobenzaldehyde

To a solution of 3-bromo-2-fluoro-6-hydroxybenzaldehyde (500 mg, 2.283 mmol) in CH₃CN (20 mL) was added BnBr (585.72 mg, 3.425 mmol), Cs₂CO₃ (2.23 g, 6.849 mmol). The resulting mixture was stirred at rt for overnight. LCMS showed the desired product was generated. The reaction was quenched with H₂O (50 mL). The resulting mixture was extracted with EtOAc (3×50 mL). The organic layers were combined, dried over Na₂SO₄, filtered and concentrated. The residue obtained was purified by silica gel chromatography (0-15% EtOAc/petroleum ether) to afford 6-(benzyloxy)-3-bromo-2-fluorobenzaldehyde as a yellow solid (700 mg, 99.18% yield). MS (ESI) calculated for C₁₄H₁₀BrFO_{2-: 309.15} m/z, found: 310.20 [M+H]⁺.

### Step 2: Synthesis of 6-(benzyloxy)-2-fluoro-3-(4,4,5,5-tetramethyl-1,3,2-dioxaborolan-2-yl)benzaldehyde

To a solution of 6-(benzyloxy)-3-bromo-2-fluorobenzaldehyde (500 mg, 1.617 mmol, 1 equiv) and bis(pinacolato)diboron (821.46 mg, 3.234 mmol, 2 equiv) in 1,4-dioxane (10 mL, 0.114 mmol, 0.07 equiv) were added KOAc (317.47 mg, 3.234 mmol, 2 equiv) and Pd(dppf)Cl₂ (118.35 mg, 0.162 mmol, 0.1 equiv). After stirring for overnight at 80° C. under a nitrogen atmosphere, the resulting mixture was concentrated under reduced pressure. The residue was purified by reverse-phase flash chromatography with the following conditions: Column, Cat No: SO230120˜2, C18, 120 g, 20˜45 μm, 100 Å; mobile phase, CH₃CN:H₂O (0.05% NH₄HCO₃)=20% increased to CH₃CN:H₂O (0.05% NH₄HCO₃)=70% in 40 min, hold CH₃CN:H₂O (0.05% NH₄HCO₃)=70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 6-(benzyloxy)-2-fluoro-3-(4,4,5,5-tetramethyl-1,3,2-dioxaborolan-2-yl)benzaldehyde (400 mg, 69.43%) as a yellow solid. MS (ESI) calculated for C₂₀H₂₂BFO₄: 356.16 m/z, found: 357.15 [M+H]⁺.

### Step 3: Synthesis of 6-(benzyloxy)-2-fluoro-3-(2-((2-methoxy-4-(4-(4-methylpiperazin-1-yl)piperidin-1-yl)phenyl)amino)-4-(phenylamino)pyrimidin-S-yl)benzaldehyde

To a solution of 6-(benzyloxy)-2-fluoro-3-(4,4,5,5-tetramethyl-1,3,2-dioxaborolan-2-yl)benzaldehyde (300 mg, 0.842 mmol) in 1,4-dioxane (4 mL) and H₂O (1 mL) was added 5-bromo-N2-{2-methoxy-4-[4-(4-methylpiperazin-1-yl)piperidin-1-yl]phenyl}-N4-phenylpyrimidine-2,4-diamine (698.02 mg, 1.263 mmol), Pd(dtbpf)Cl₂ (54.89 mg, 0.084 mmol), K₃PO₄ (446.94 mg, 2.105 mmol). The resulting mixture was maintained under nitrogen and stirred at 90° C. for 3 h. LCMS showed the desired product was generated. After cooling down to rt, the reaction was quenched with H₂O (50 mL). The resulting mixture was extracted with EtOAc (3×50 mL). The organic layers were combined, dried over Na₂SO₄, filtered and concentrated. The crude product was purified by reverse-phase flash chromatography with the following conditions: Column, Cat No: 50230120˜2, C18, 120 g, 20˜45 μm, 100 Å; mobile phase, CH₃CN:H₂O (0.05% FA)=20% increased to CH₃CN:H₂O (0.05% FA)=70% in 40 min, hold CH₃CN:H₂O (0.05% FA)=70% in 10 min, up to CH₃CN:H₂O (0.05% FA)=95% in 2 min, hold CH₃CN:H₂O (0.05% FA)=95% in 10 min; Detector, UV 220 nm & 254 nm. After lyophilization, it was afforded the 6-(benzyloxy)-2-fluoro-3-[2-({2-methoxy-4-[4-(4-methylpiperazin-1-yl)piperidin-1-yl]phenyl}amino)-4-(phenylamino)pyrimidin-S-yl]benzaldehyde as a yellow solid (140 mg, 23.68% yield). MS (ESI) calculated for C₄₁H₄₄FN₇O₃: 701.15 m/z, found: 702.20 [M+H]⁺.

### Step 4: Synthesis of 2-fluoro-6-hydroxy-3-(2-((2-methoxy-4-(4-(4-methylpiperazin-1-yl)piperidin-1-yl)phenyl)amino)-4-(phenylamino)pyrimidin-S-yl)benzaldehyde

To a solution of 6-(benzyloxy)-2-fluoro-3-[2-({2-methoxy-4-[4-(4-methylpiperazin-1-yl)piperidin-1-yl]phenyl}amino)-4-(phenylamino)pyrimidin-S-yl]benzaldehyde (150 mg, 0.214 mmol) in TFA (3 mL) and methanesulfonic acid (0.6 mL). The resulting mixture was stirred at rt for 1 h. The reaction was quenched with H₂O (10 mL). The pH value of the aqueous phase was adjusted to 7-8 used NaHCO₃ solution. The resulting mixture was extracted with EtOAc (3×20 mL). The organic layers were combined, dried over Na₂SO₄, filtered and concentrated. The crude product was then purified by Prep-HPLC with the following conditions (1 #-Waters 2767-5): Column, SunFire Prep C18, 5 μm, 19*100 mm; mobile phase, H₂O (0.05% NH₄HCO₃) and CH₃CN (65% CH₃CN up to 75% in 11 min, up to 100% in 1 min, hold 100% in 1 min, down to 65% in 1 min, hold 65% in 1 min); Detector, UV 220&254 nm. After lyophilization, it was afforded the 2-fluoro-6-hydroxy-3-[2-({2-methoxy-4-[4-(4-methylpiperazin-1-yl)piperidin-1-yl]phenyl}amino)-4-(phenylamino)pyrimidin-S-yl]benzaldehyde as a yellow solid (6.6 mg, 4.21% yield). MS (ESI) calculated for C₃₄H₃₈FN₇O₃, 611.30 m/z, found: 612.30 [M+H]⁺. ¹H-NMR (300 MHz, DMSO-d6) δ (ppm): 10.33 (s, 1H), 8.16 (s, 1H), 7.83 (s, 1H), 7.73 (s, 1H), 7.64-7.66 (m, 1H), 7.46-7.57 (m, 3H), 7.21-7.31 (m, 2H), 6.91-7.08 (m, 2H), 6.63 (s, 1H), 6.40-6.44 (m, 1H), 3.80 (s, 3H), 3.63-3.74 (m, 2H), 3.35 (s, 2H), 2.62-2.95 (m, 12H), 1.91-1.98 (m, 2H), 1.55 (s, 2H).

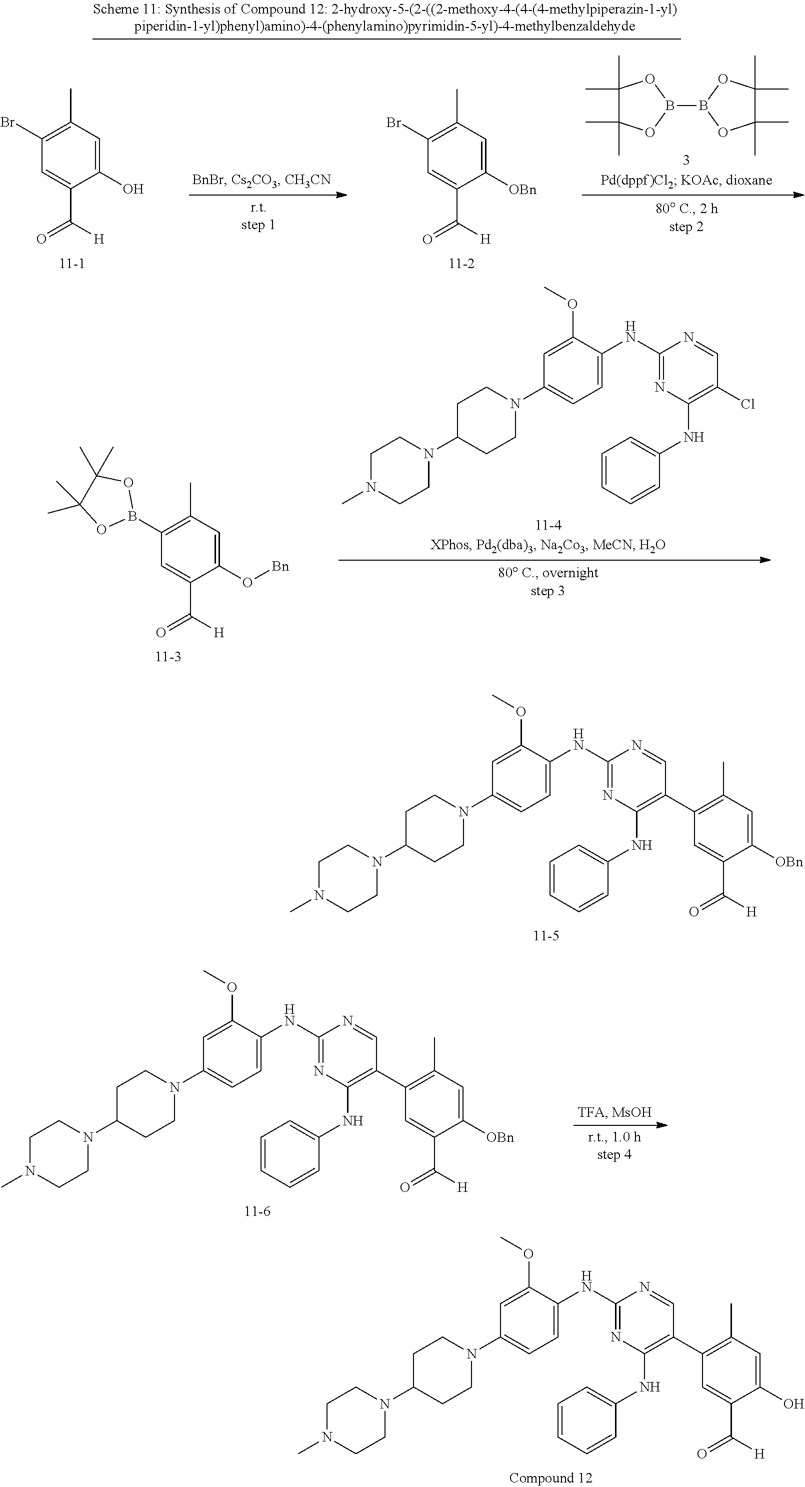

### Step 1: Synthesis of 5-bromo-2-hydroxy-4-methylbenzaldehyde

To a solution of 5-bromo-2-hydroxy-4-methoxybenzaldehyde (488 mg, 2.283 mmol) in CH₃CN (20 mL) was added BnBr (585.72 mg, 3.425 mmol), Cs₂CO₃ (2.23 g, 6.849 mmol). The resulting mixture was stirred at rt for overnight. LCMS showed the desired product was generated. The reaction was quenched with H₂O (50 mL). The resulting mixture was extracted with EtOAc (3×50 mL). The organic layers were combined, dried over Na₂SO₄, filtered and concentrated. The residue obtained was purified by silica gel chromatography (0-15% EtOAc/petroleum ether) to afford 5-bromo-2-hydroxy-4-methylbenzaldehyde as a yellow solid (490 mg, 70.60% yield). MS (ESI) calculated for C₁₅H₁₃BrO_{2-: 304.01} m/z, found: 305.10 [M+H]⁺.

### Step 2: Synthesis of 2-(benzyloxy)-4-methyl-S-(4,4,5,5-tetramethyl-1,3,2-dioxaborolan-2-yl)benzaldehyde

To a solution of 5-bromo-2-hydroxy-4-methylbenzaldehyde (491.5 mg, 1.617 mmol, 1 equiv) and bis(pinacolato)diboron (821.46 mg, 3.234 mmol, 2 equiv) in 1,4-dioxane (10 mL) were added KOAc (317.47 mg, 3.234 mmol, 2 equiv) and Pd(dppf)Cl₂ (118.35 mg, 0.162 mmol, 0.1 equiv). After stirring for overnight at 80° C. under a nitrogen atmosphere, the resulting mixture was concentrated under reduced pressure. The residue was purified by reverse-phase flash chromatography with the following conditions: Column, Cat No: 50230120˜2, C18, 120 g, 20˜45 μm, 100 Å, mobile phase, CH₃CN:H₂O (0.05% NH₄HCO₃) -20% increased to CH₃CN:H₂O (0.05% NH₄HCO₃)=70% in 40 min, hold CH₃CN:H₂O (0.05% NH₄HCO₃)=70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 2-(benzyloxy)-4-methyl-S-(4,4,5,5-tetramethyl-1,3,2-dioxaborolan-2-yl)benzaldehyde (450 mg, 79.02% yield) as a yellow solid. MS (ESI) calculated for C₂₁H₂₅BO₄: 352.18 m/z, found: 353.15 [M+H]⁺.

### Step 3: Synthesis of 2-(benzyloxy)-S-[2-({2-methoxy-4-[4-(4-methylpiperazin-1-yl)piperidin-1-yl]phenyl}amino)-4-(phenylamino)pyrimidin-S-yl]-4-methylbenzaldehyde

5-chloro-N2-{2-methoxy-4-[4-(4-methylpiperazin-1-yl)piperidin-1-yl]phenyl}-N4-phenylpyrimidine-2,4-diamine (500 mg, 0.984 mmol, 1 equiv), a stir bar and ACN (8 mL) and H₂O (2 mL) were added to a 50-mL round-bottom flask and stirred until homogeneous. 2-(benzyloxy)-4-methyl-S-(4,4,5,5-tetramethyl-1,3,2-dioxaborolan-2-yl)benzaldehyde (519.97 mg, 1.476 mmol, 1.5 equiv), XPhos (46.92 mg, 0.098 mmol, 0.1 equiv), pd₂(dba)₃ (90.12 mg, 0.098 mmol, 0.1 equiv) and Na₂CO₃ (208.61 mg, 1.968 mmol, 2 equiv) were added to the reaction flask at rt. The resulting mixture was stirred at 80° C. for overnight. The crude product was purified by reverse-phase flash chromatography with the following conditions: Column, Cat No: SO230120-2, C18, 120 g, 20˜45 μm, 100 Å; mobile phase, CH₃CN:H₂O (0.05% FA)=20% increased to CH₃CN:H₂O (0.05% FA)=70% in 40 mn, hold CH₃CN:H₂O (0.05% FA)=70% in 10 min, up to CH₃CN:H₂O (0.05% FA)=95% in 2 min, hold CH₃CN:H₂O (0.05% FA)=95% in 10 min; Detector, UV 220 nm & 254 nm. After lyophilization, it was afforded the 2-(benzyloxy)-S-[2-({2-methoxy-4-[4-(4-methylpiperazin-1-yl)piperidin-1-yl]phenyl}amino)-4-(phenylamino)pyrimidin-S-yl]-4-methylbenzaldehyde (300 mg, 43.68%) as a yellow solid. MS (ESI) calculated for C₄₂H₄₇N₇O₃, 697.37 m/z, found 698.35 [M+H]⁺.

### Step 4: Synthesis of 2-hydroxy-S-[2-({2-methoxy-4-[4-(4-methylpiperazin-1-yl)piperidin-1-yl]phenyl}amino)-4-(phenylamino)pyrimidin-S-yl]-4-methylbenzaldehyde

2-(benzyloxy)-S-[2-({2-methoxy-4-[4-(4-methylpiperazin-1-yl)piperidin-1-yl]phenyl}amino)-4-(phenylamino)pyrimidin-S-yl]-4-methylbenzaldehyde (100 mg, 0.143 mmol, 1 equiv), a stir bar and TFA (2 mL) were added to a 50-mL round-bottom flask and stirred until homogeneous. Methanesulfonic acid (0.4 mL) was added to the reaction flask at rt. The resulting mixture was stirred at rt for 1 h. The residue was diluted with water, then adjusted to pH 6-7 with NaHCO₃, dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The crude product was then purified by Prep-HPLC with the following conditions: Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 25% B to 55% B in 9 min, 55% B; Wave Length: 254 nm; RT1(min): 7; Number of Runs: 0. After lyophilization, it was afforded the 2-hydroxy-S-[2-({2-methoxy-4-[4-(4-methylpiperazin-1-yl)piperidin-1-yl]phenyl}amino)-4-(phenylamino)pyrimidin-S-yl]-4-methylbenzaldehyde (15.4 mg, 17.60%) as a light yellow solid. MS (ESI) calculated for C₃₅H₄₁N₇O₃, 607.33 m/z, found 608.40 [M+H]⁺.¹H NMR (400 MHz, DMSO-d₆) δ (ppm): 10.76 (brs, 1H), 10.22 (s, 1H), 7.83 (s, 1H), 7.67-7.77 (m, 2H), 7.47-7.64 (m, 4H), 7.17-7.26 (m, 2H), 6.93-7.03 (m, 2H), 6.59-6.67 (m, 1H), 6.36-6.42 (m, 1H), 3.80 (s, 3H), 3.36-3.71 (m, 2H), 2.57-2.68 (m, 3H), 2.50-2.55 (m, 4H), 2.22-2.46 (m, 4H), 2.12-2.17 (m, 6H), 1.81-1.89 (m, 2H), 1.44-1.59 (m, 2H).

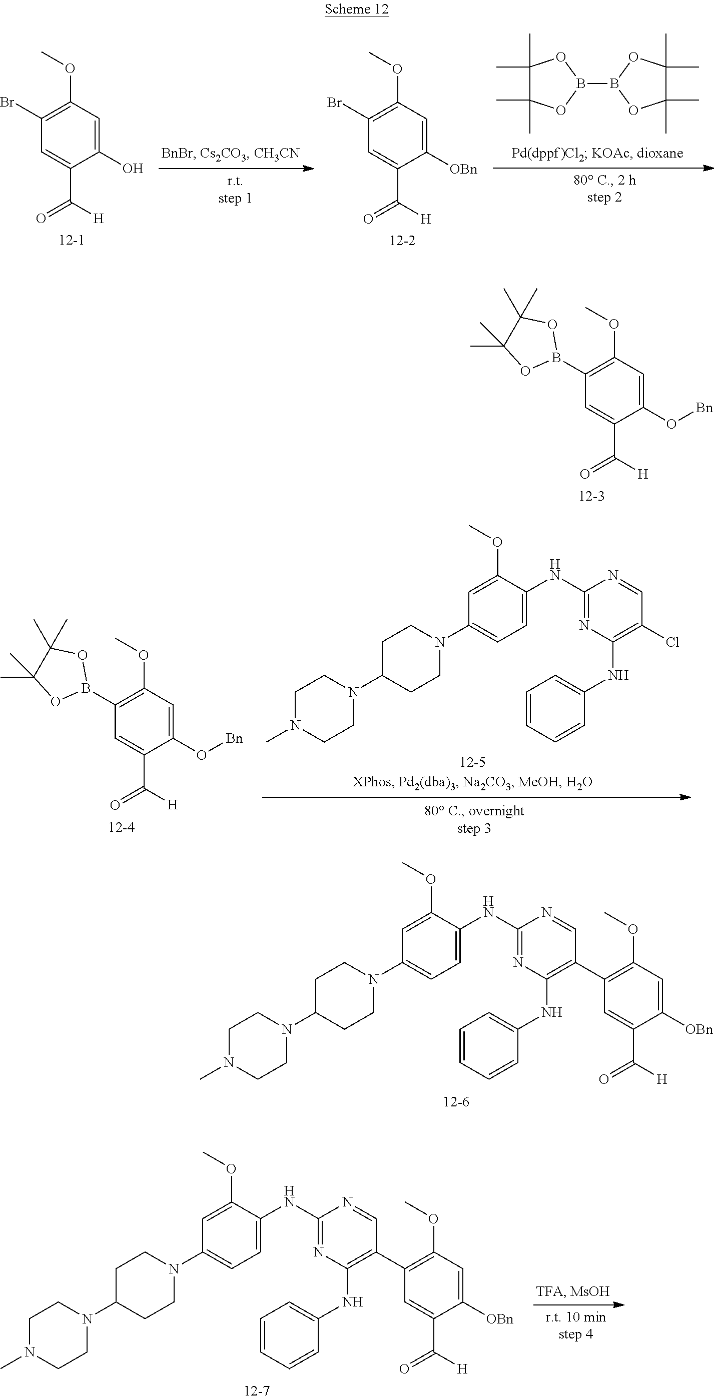

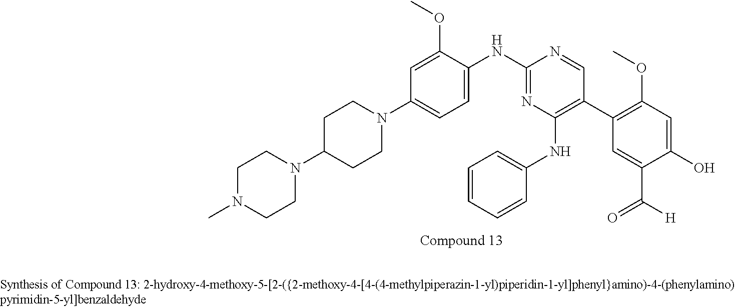 [SMILES: COc1cc(N2CCC(N3CCN(C)CC3)CC2)ccc1Nc1ncc(-c2cc(C=O)c(O)cc2OC)c(Nc2ccccc2)n1]

### Step 1: Synthesis of 2-(benzyloxy)-S-bromo-4-methoxybenzaldehyde

To a solution of 5-bromo-2-hydroxy-4-methoxybenzaldehyde (525 mg, 2.283 mmol) in CH₃CN (20 mL) was added BnBr (585.72 mg, 3.425 mmol), Cs₂CO₃ (2.23 g, 6.849 mmol). The resulting mixture was stirred at rt for overnight. LCMS showed the desired product was generated. The reaction was quenched with H₂O (50 mL). The resulting mixture was extracted with EtOAc (3×50 mL). The organic layers were combined, dried over Na₂SO₄, filtered and concentrated. The residue obtained was purified by silica gel chromatography (0-15% EtOAc/petroleum ether) to afford 2-(benzyloxy)-S-bromo-4-methoxybenzaldehyde as a yellow solid (590 mg, 80.76% yield). MS (ESI) calculated for C₁₅H₁₃BrO₃: 320.00 m/z, found: 321.10 [M+H]⁺.

### Step 2: Synthesis of 2-(benzyloxy)-4-methoxy-S-(4,4,5,5-tetramethyl-1,3,2-dioxaborolan-2-yl)benzaldehyde

To a solution of 2-(benzyloxy)-S-bromo-4-methoxybenzaldehyde (518.0 mg, 1.617 mmol, 1 equiv) and bis(pinacolato)diboron (821.46 mg, 3.234 mmol, 2 equiv) in 1,4-dioxane (10 mL) were added KOAc (317.47 mg, 3.234 mmol, 2 equiv) and Pd(dppf)Cl₂ (118.35 mg, 0.162 mmol, 0.1 equiv). After stirring for overnight at 80° C. under a nitrogen atmosphere, the resulting mixture was concentrated under reduced pressure. The residue was purified by reverse-phase flash with the following conditions: Column, Cat No: 50230120˜2, C18, 120 g, 20˜45 μm, 100 Å; mobile phase, CH₃CN:H₂O (0.05% NH₄HCO₃)=20% increased to CH₃CN:H₂O (0.05% NH₄HCO₃)=70% in 40 min, hold CH₃CN:H₂O (0.05% NH₄HCO₃) -70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 2-(benzyloxy)-4-methoxy-S-(4,4,5,5-tetramethyl-1,3,2-dioxaborolan-2-yl)benzaldehyde (550 mg, 92.38% yield) as a yellow solid. MS (ESI) calculated for C₂₁H₂₅BO₅: 368.18 m/z, found: 369.15 [M+H].

### Step 3: Synthesis of 2-(benzyloxy)-4-methoxy-S-[2-({2-methoxy-4-[4-(4-methylpiperazin-1-yl) piperidin-1-yl] phenyl} amino)-4-(phenylamino)pyrimidin-S-yl]benzaldehyde

5-chloro-N2-{2-methoxy-4-[4-(4-methylpiperazin-1-yl)piperidin-1-yl]phenyl}-N4-phenylpyrimidine-2,4-diamine (500 mg, 0.984 mmol, 1 equiv), a stir bar and ACN (8 mL) and H₂O (2 mL) were added to a 50-mL round-bottom flask and stirred until homogeneous. 2-(benzyloxy)-4-methoxy-S-(4,4,5,5-tetramethyl-1,3,2-dioxaborolan-2-yl)benzaldehyde (543.59 mg, 1.476 mmol, 1.5 equiv), XPhos (46.92 mg, 0.098 mmol, 0.1 equiv), Pd₂(dba)₃ (90.12 mg, 0.098 mmol, 0.1 equiv) and Na₂CO₃ (208.61 mg, 1.968 mmol, 2 equiv) were added to the reaction flask at rt. The resulting mixture was stirred at 80° C. for overnight. The crude product was purified by reverse-phase flash chromatography with the following conditions: Column, Cat No: SO230120-2, C18, 120 g, 20˜45 μm, 100 Å; mobile phase, CH₃CN:H₂O (0.05% FA)=20% increased to CH₃CN:H₂O (0.05% FA)=70% in 40 mn, hold CH₃CN:H₂O (0.05% FA)=70% in 10 min, up to CH₃CN:H₂O (0.05% FA)=95% in 2 min, hold CH₃CN:H₂O (0.05% FA)=95% in 10 min; Detector, UV 220 nm & 254 nm. After lyophilization, it was afforded the 2-(benzyloxy)-4-methoxy-S-[2-({2-methoxy-4-[4-(4-methylpiperazin-1-yl)piperidin-1-yl]phenyl}amino)-4-(phenylamino)pyrimidin-S-yl]benzaldehyde (300 mg, 42.70% yield) as a yellow solid. MS (ESI) calculated for C₄₂H₄₇N₇O₄, 713.37 m/z, found 714.35 [M+H]⁺.

### Step 4: Synthesis of 2-hydroxy-4-methoxy-S-[2-({2-methoxy-4-[4-(4-methylpiperazin-1-yl)piperidin-1-yl]phenyl} amino)-4-(phenylamino)pyrimidin-S-yl]benzaldehyde

2-(benzyloxy)-4-methoxy-S-[2-({2-methoxy-4-[4-(4-methylpiperazin-1-yl)piperidin-1-yl]phenyl}amino)-4-(phenylamino)pyrimidin-S-yl]benzaldehyde (100 mg, 0.140 mmol, 1 equiv), a stir bar and TFA (2 mL) were added to a 50-mL round-bottom flask and stirred until homogeneous. Methanesulfonic acid (0.4 mL) was added to the reaction flask at rt. The resulting mixture was stirred at rt for 10 min. The residue was diluted with water, then adjusted to pH 6-7 with NaHCO₃, dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The crude product was then purified by Prep-HPLC with the following conditions: Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 25% B to 55% B in 9 min, 55% B; Wave Length: 254 nm; RT1(min): 7; Number Of Runs. After lyophilization, it was afforded the 2-hydroxy-4-methoxy-S-[2-({2-methoxy-4-[4-(4-methylpiperazin-1-yl)piperidin-1-yl]phenyl}amino)-4-(phenylamino)pyrimidin-S-yl]benzaldehyde (14.0 mg, 15.66% yield) as a yellow solid. MS (ESI) calculated for C₃₅H₄₁N₇O₄, 623.32 m/z, found 624.40 [M+H]⁺.¹H NMR (300 MHz, DMSO-d₆) δ (ppm): 8.26 (s, 1H), 8.10-8.20 (m, 1H), 7.66-7.75 (m, 2H), 7.47-7.56 (m, 1H), 7.24-7.44 (m, 3H), 7.07 (t, J=8.7 Hz, 1H), 4.94-5.10 (m, 1H), 3.99-4.16 (m, 1H), 3.47 (s, 3H), 3.20-3.31 (m, 1H), 3.10-3.20 (m, 1H), 2.78-2.99 (m, 2H), 2.66-2.78 (m, 1H).

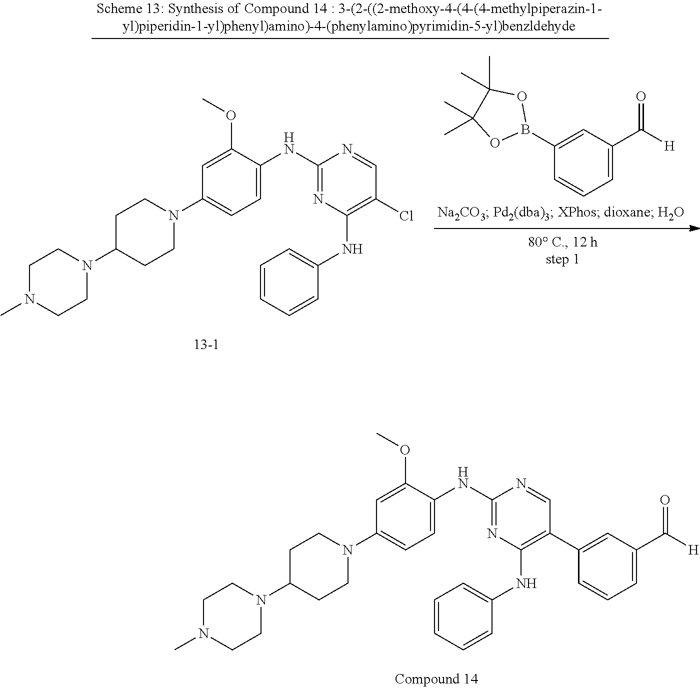 [SMILES: COc1cc(N2CCC(N3CCN(C)CC3)CC2)ccc1Nc1ncc(Cl)c(Nc2ccccc2)n1 ; CC1(C)OB(c2cccc(C=O)c2)OC1(C)C ; COc1cc(N2CCC(N3CCN(C)CC3)CC2)ccc1Nc1ncc(-c2cccc(C=O)c2)c(Nc2ccccc2)n1]

### Step 1: Synthesis of 4′-((2-methoxy-4-(4-(4-methylpiperazin-1-yl)piperidin-1-yl)phenyl)amino)-2′-(phenylamino)-[1,1′-biphenyl]-3-carbaldehyde

5-chloro-N2-{2-methoxy-4-[4-(4-methylpiperazin-1-yl)piperidin-1-yl]phenyl}-N4-phenylpyrimidine-2,4-diamine (50 mg, 0.098 mmol) in dioxane (2 mL) and H₂O (0.5 mL) was added 3-(4,4,5,5-tetramethyl-1,3,2-dioxaborolan-2-yl)benzaldehyde (4 equiv),Pd₂(dba)₃ (0.1 equiv), XPhos (0.4 equiv), Na₂CO₃ (3 equiv). The resulting mixture was maintained under nitrogen and stirred at 90° C. for overnight. The crude product was then purified by Prep-HPLC with the following conditions: Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 17% B in 8 min, 17% B; Wave Length: 254/220 nm; RT1(min): 8; After lyophilization, it was afforded the 3-[2-({2-methoxy-4-[4-(4-methylpiperazin-1-yl)piperidin-1-yl]phenyl}amino)-4-(phenylamino)pyrimidin-S-yl]benzaldehyde as an yellow solid (27.7 mg, 8.27% yield). MS (ESI) calculated for C₃₄H₃₉N₇O_{2-: 577.33} m/z, found: 578.30[M+H]⁺. Found: ¹H NMR (400 MHz, DMSO-d6) δ ppm: 10.08 (s, 1H), 8.22-8.30 (m, 1H), 7.88-8.00 (m, 3H), 7.64-7.80 (m, 4H), 7.54-7.56 (m, 2H), 7.21-7.25 (m, 2H), 6.99-7.02 (m, 1H), 6.62 (s, 1H), 6.38-6.40 (m, 1H), 3.80 (m, 3H), 3.66-3.69 (m, 3H), 2.61-2.66 (m, 2H), 2.50 (s, 3H), 2.29-2.33 (m, 5H), 2.16 (s, 3H), 1.83-1.85 (m, 2H), 1.51-1.56 (m, 2H).

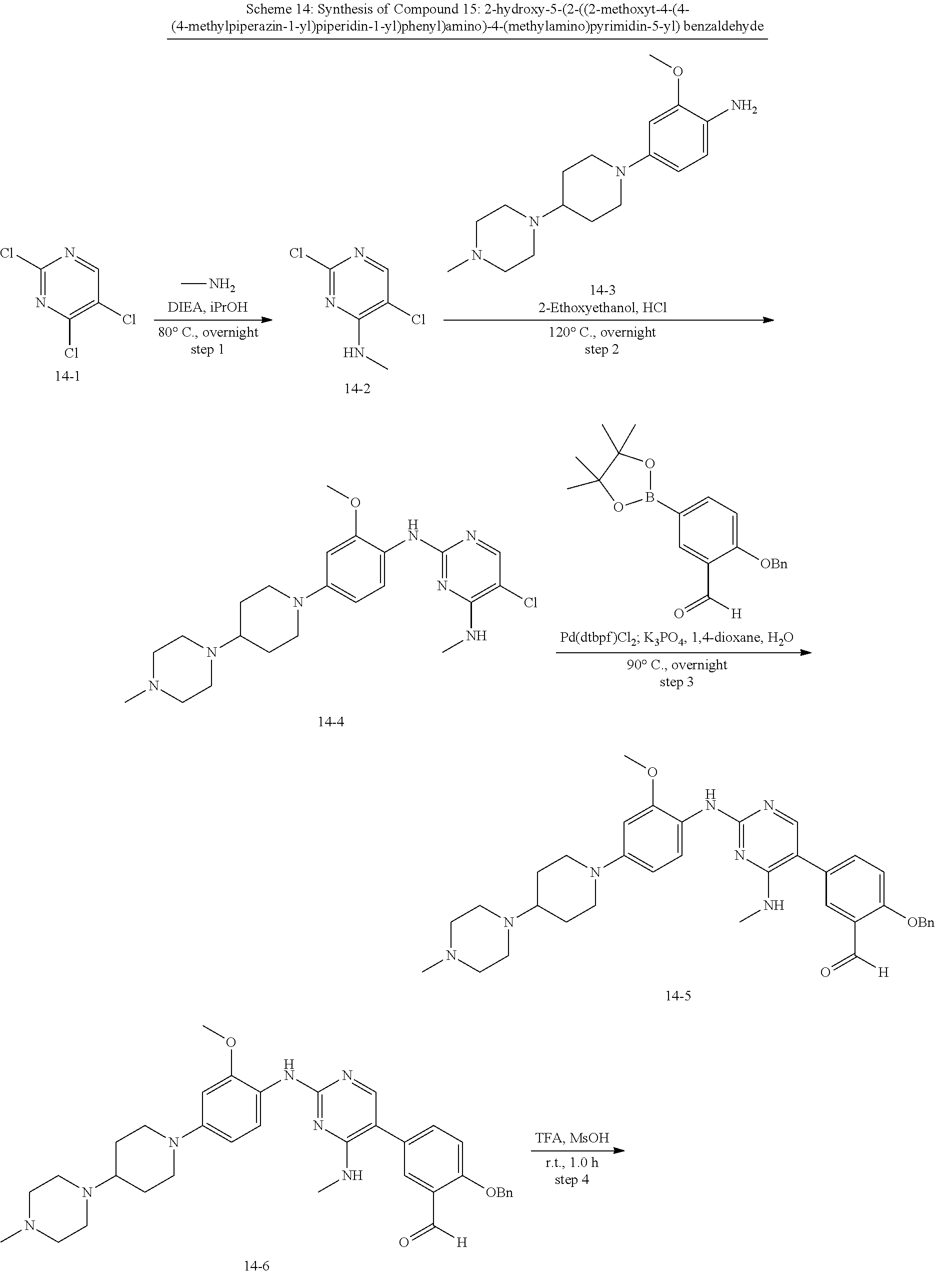

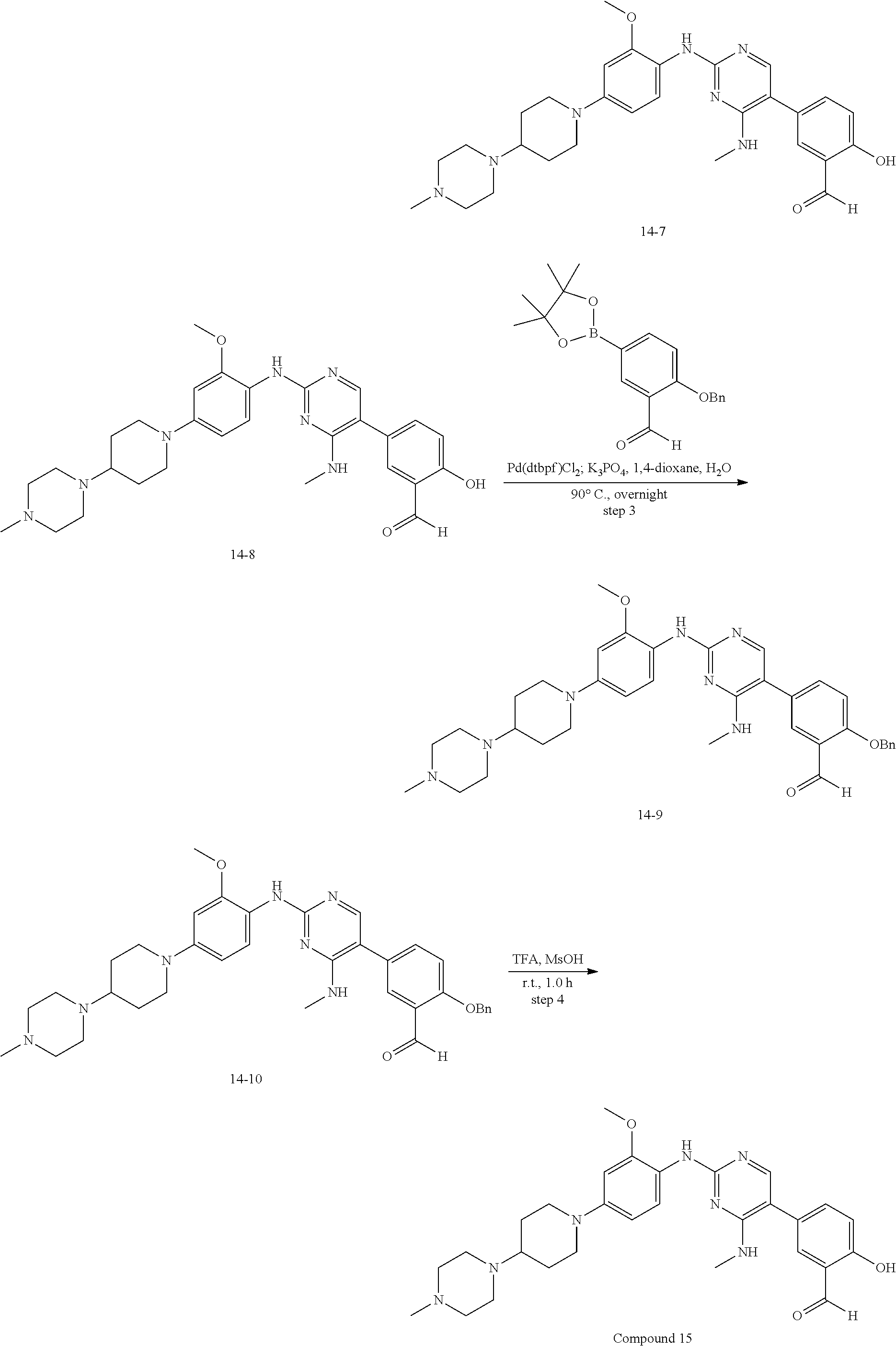 [SMILES: CNc1nc(Nc2ccc(N3CCC(N4CCN(C)CC4)CC3)cc2OC)ncc1-c1ccc(O)c(C=O)c1 ; CNc1nc(Nc2ccc(N3CCC(N4CCN(C)CC4)CC3)cc2OC)ncc1-c1ccc(O)c(C=O)c1 ; CC1(C)OB(c2ccc(OCc3ccccc3)c(C=O)c2)OC1(C)C ; CNc1nc(Nc2ccc(N3CCC(N4CCN(C)CC4)CC3)cc2OC)ncc1-c1ccc(OCc2ccccc2)c(C=O)c1 ; CNc1nc(Nc2ccc(N3CCC(N4CCN(C)CC4)CC3)cc2OC)ncc1-c1ccc(OCc2ccccc2)c(C=O)c1 ; CNc1nc(Nc2ccc(N3CCC(N4CCN(C)CC4)CC3)cc2OC)ncc1-c1ccc(O)c(C=O)c1]

### Step 1: Synthesis of 2,5-dichloro-N-methylpyrimidin-4-amine

To a solution of 2,4,5-trichloropyrimidine (400 mg, 2.181 mmol) in 2-Propanol (5 mL) was added methylamine (74.50 mg, 2.399 mmol), DIEA (422.79 mg, 3.272 mmol). The resulting mixture was maintained under nitrogen and stirred at 90° C. for overnight. LCMS showed the desired product was generated. The reaction was concentrated. The residue obtained was purified by silica gel chromatography (0-20% EtOAc/petroleum ether) to afford 2,5-dichloro-N-methylpyrimidin-4-amine as a yellow oil (320 mg, 82.43% yield). MS (ESI) calculated for C₅H₅C₁₂N₃: 178.02 m/z, found: 179.10 [M+H].

### Step 2: Synthesis of 5-chloro-N2-(2-methoxy-4-(4-(4-methylpiperazin-1-yl)piperidin-1-yl)phenyl)-N4-methylpyrimidine-2,4-diamine

To a solution of 2,5-dichloro-N-methylpyrimidin-4-amine (220 mg, 1.236 mmol) in 2-Ethoxyethanol (6 mL) was added 2-methoxy-4-[4-(4-methylpiperazin-1-yl)piperidin-1-yl]aniline (376.23 mg, 1.236 mmol), HCl(g) in MeOH (0.5 mL)(4M). The resulting mixture was maintained under nitrogen and stirred at 120° C. for overnight. LCMS showed the desired product was generated. After cooling down to rt, the reaction was quenched with H₂O (50 mL). The pH value of the aqueous phase was adjusted to 7-8 used NaHCO₃ solution. The resulting mixture was extracted with EtOAc (3×50 mL). The organic layers were combined, dried over Na₂SO₄, filtered and concentrated. The crude product was purified by reverse-phase flash with the following conditions: Column, Cat No: 50230120-2, C18, 120 g, 20˜45 μm, 100 Å; mobile phase, CH₃CN:H₂O (0.05% NH₄HCO₃)=20% increased to CH₃CN:H₂O (0.05% NH₄HCO₃) -70% in 40 min, hold CH₃CN:H₂O (0.05% NH₄HCO₃)=70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 5-chloro-N2-{2-methoxy-4-[4-(4-methylpiperazin-1-yl)piperidin-1-yl]phenyl}-N4-methylpyrimidine-2,4-diamine as a yellow solid (300 mg, 54.43% yield). MS (ESI) calculated for C₂₂H₃₂ClN₇O: 445.24 m/z, found: 446.20 [M+H]⁺.

### Step 3: Synthesis of 2-(benzyloxy)-S-(2-((2-methoxy-4-(4-(4-methylpiperazin-1-yl)piperidin-1-yl)phenyl)amino)-4-(methylamino)pyrimidin-S-yl)benzaldehyde

To a solution of 5-chloro-N2-{2-methoxy-4-[4-(4-methylpiperazin-1-yl)piperidin-1-yl]phenyl}-N4-methylpyrimidine-2,4-diamine (300 mg, 0673 mmol) in CH₃CN (8 mL) and H₂O (2 mL) was added 2-(benzyloxy)-S-(4,4,5,5-tetramethyl-1,3,2-dioxaborolan-2-yl)benzaldehyde (341.24 mg, 1.010 mmol), Pd₂(dba)₃ (61.60 mg, 0.067 mmol), Xphos (32.07 mg, 0.067 mmol), Na₂CO₃ (142.58 mg, 1.346 mmol). The resulting mixture was maintained under nitrogen and stirred at 90° C. overnight. LCMS showed the desired product was generated. After cooling down to rt, the reaction was quenched with H₂O (50 mL). The resulting mixture was extracted with EtOAc (3×50 mL). The organic layers were combined, dried over Na₂SO₄, filtered and concentrated. The crude product was purified by reverse-phase flash chromatography with the following conditions: Column, Cat No: SO230120˜2, C18, 120 g, 20˜45 μm, 100 Å; mobile phase, CH₃CN:H₂O (0.05% NH₄HCO₃)=20% increased to CH₃CN:H₂O (0.05% NH₄HCO₃)=70% in 40 min, hold CH₃CN:H₂O (0.05% NH₄HCO₃) -70% in 10 min, up to CH₃CN:H₂O (0.05% NH₄HCO₃)=95% in 2 min, hold CH₃CN:H₂O (0.05% NH₄HCO₃)=95% in 10 min; Detector, UV 220 nm & 254 nm. After lyophilization, it was afforded the 2-(benzyloxy)-S-[2-({2-methoxy-4-[4-(4-methylpiperazin-1-yl)piperidin-1-yl]phenyl}amino)-4-(methylamino)pyrimidin-S-yl]benzaldehyde as a yellow solid (200 mg, 47.82% yield). MS (ESI) calculated for C₃₆H₄₃N₇O₃: 621.34 m/z, found: 622.70 [M+H]⁺.

### Step 4: Synthesis of 2-hydroxy-S-(2-((2-methoxy-4-(4-(4-methylpiperazin-1-yl)piperidin-1-yl)phenyl)amino)-4-(methylamino)pyrimidin-S-yl)benzaldehyde

To a solution of 2-hydroxy-S-[2-({2-methoxy-4-[4-(4-methylpiperazin-1-yl)piperidin-1-yl]phenyl}amino)-4-(methylamino)pyrimidin-S-yl]benzaldehyde (200 mg, 0322 mmol) in TFA (5 mL) and methanesulfonic acid (1 mL). The resulting mixture was stirred at rt for 1 h. The reaction was quenched with H₂O (10 mL). The pH value of the aqueous phase was adjusted to 7-8 using a NaHCO₃ solution. The resulting mixture was extracted with EtOAc (3×20 mL).

The organic layers were combined, dried over Na₂SO₄, filtered and concentrated. The crude product was then purified by Prep-HPLC with the following conditions (1 #-Waters 2767-5): Column, SunFire Prep C18, 5 μm, 19*100 mm; mobile phase, H₂O (0.05% NH₄HCO₃) and CH₃CN (65% CH3CN up to 75% in 11 min, up to 100% in 1 min, hold 100% in 1 min, down to 65% in 1 min, hold 65% in 1 min); Detector, UV 220&254 nm. After lyophilization, it was afforded the 2-hydroxy-S-[2-({2-methoxy-4-[4-(4-methylpiperazin-1-yl)piperidin-1-yl]phenyl}amino)-4-(methylamino)pyrimidin-S-yl]benzaldehyde as a yellow solid (76.5 mg, 44.56% yield). MS (ESI) calculated for C₂₉H₃₇N₇O₃: 531.30 m/z, found: 532.30 [M+H]⁺. ¹H-NMR (300 MHz, DMSO-d6) δ (ppm): 11.17-11.22 (m, 1H), 10.33 (s, 1H), 9.63 (s, 1H), 7.95 (s, 1H), 7.44-7.62 (m, 4H), 7.13-7.16 (m, 1H), 6.74 (s, 1H), 6.60-6.62 (m, 1H), 3.84-3.94 (m, 6H), 3.39-3.77 (m, 4H), 2.95-3.32 (m, 4H), 2.86-2.87 (m, 6H), 2.70-2.78 (m, 2H), 2.00-2.15 (m, 2H), 1.65-1.75 (m, 2H).

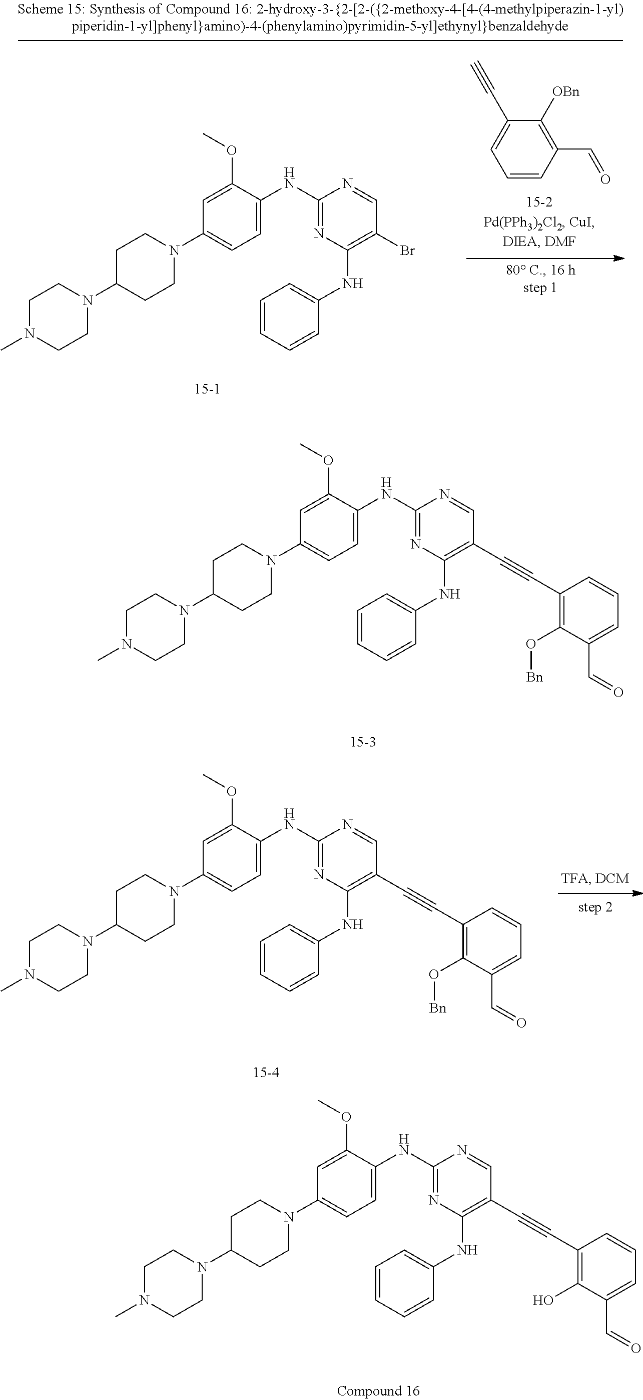 [SMILES: COc1cc(N2CCC(N3CCN(C)CC3)CC2)ccc1Nc1ncc(Br)c(Nc2ccccc2)n1 ; C#Cc1cccc(C=O)c1OCc1ccccc1 ; COc1cc(N2CCC(N3CCN(C)CC3)CC2)ccc1Nc1ncc(C#Cc2cccc(C=O)c2OCc2ccccc2)c(Nc2ccccc2)n1 ; COc1cc(N2CCC(N3CCN(C)CC3)CC2)ccc1Nc1ncc(C#Cc2cccc(C=O)c2OCc2ccccc2)c(Nc2ccccc2)n1 ; COc1cc(N2CCC(N3CCN(C)CC3)CC2)ccc1Nc1ncc(C#Cc2cccc(C=O)c2O)c(Nc2ccccc2)n1]

### Step 1: Synthesis of 2-(benzyloxy)-3-{2-[2-({2-methoxy-4-[4-(4-methylpiperazin-1-yl)piperidin-1-yl]phenyl}amino)-4-(phenylamino)pyrimidin-S-yl]ethynyl}benzaldehyde

To a solution of 5-bromo-N2-{2-methoxy-4-[4-(4-methylpiperazin-1-yl)piperidin-1-yl]phenyl}-N4-phenylpyrimidine-2,4-diamine (20 mg, 0.036 mmol, 1 equiv) and 2-(benzyloxy)-3-ethynylbenzaldehyde (9.41 mg, 0.040 mmol, 1.1 equiv) in DMF (2 mL) were added CuI (0.34 mg, 0.002 mmol, 0.05 equiv), Pd(Pph3)₂C₁₂ (1.27 mg, 0.002 mmol, 0.05 equiv) and DIEA (9.36 mg, 0.072 mmol, 2 equiv). After stirring overnight at 80° C. under a nitrogen atmosphere, the resulting mixture was concentrated under reduced pressure. The residue was purified by reverse flash chromatography with the following conditions: column, C18 silica gel; mobile phase, ACN in water, 10% to 60% gradient in 20 min; detector, UV 254 nm giving 2-(benzyloxy)-3-{2-[2-({2-methoxy-4-[4-(4-methylpiperazin-1-yl)piperidin-1-yl]phenyl}amino)-4-(phenylamino)pyrimidin-S-yl]ethynyl}benzaldehyde (140 mg, 54.64%) as light yellow solid. MS (ESI) calculated for C₄₃H₄₅N₇O₃, 707.36 m/z, found: 707.30 [M+H]⁺.

### Step 2: Synthesis of 2-hydroxy-3-{2-[2-({2-methoxy-4-[4-(4-methylpiperazin-1-yl)piperidin-1-yl]phenyl}amino)-4-(phenylamino)pyrimidin-S-yl]ethynyl}benzaldehyde

To a solution of 2-(benzyloxy)-3-{2-[2-({2-methoxy-4-[4-(4-methylpiperazin-1-yl)piperidin-1-yl]phenyl}amino)-4-(phenylamino)pyrimidin-S-yl]ethynyl}benzaldehyde (100 mg, 0.141 mmol, 1 equiv) in DCM (5 mL) was added boron trichloride (0.423 mL, 1 M in DCM). After stirring for 3 h at rt under a nitrogen atmosphere, the resulting mixture was concentrated under reduced pressure. The residue was submitted to Prep-HPLC with following conditions: Column 30*150 mm 5 μm, n; Mobile Phase A: Water(0.1% FA), Mobile Phase B: ACN; Flow rate: 60 mL/min; Gradient: 40% B to 60% B in 8 min, 60% B; Wave Length: 254/220 nm. After lyophilization, it provided 2-hydroxy-3-{2-[2-({2-methoxy-4-[4-(4-methylpiperazin-1-yl) piperidin-1-yl]phenyl}amino)-4-(phenylamino)pyrimidin-S-yl]ethynyl}benzaldehyde (5.0 mg, 5.69%) as a yellow solid (5.0 mg, 5.69% yield). MS (ESI) calculated for C₃₆H₃₉N₇O₃: 617.31, found: 618.35 [M+H]⁺. ¹H NMR (300 MHz, DMSO-d6) δppm: 10.26-10.39 (m, 1H), 9.53 (s, 1H), 8.16-8.25 (m, 1H), 8.00-8.08 (m, 1H), 7.79-7.95 (m, 2H), 7.44-7.53 (m, 3H), 7.24-7.34 (m, 3H), 6.99-7.04 (m, 1H), 6.56-6.62 (m, 2H), 6.45-6.48 (m, 1H), 3.71-3.82 (m, 6H), 2.66-2.71 (m, 5H), 2.37-2.40 (m, 2H), 2.27-2.32 (m, 4H), 1.85-1.88 (m, 3H), 1.52-1.58 (m, 3H).

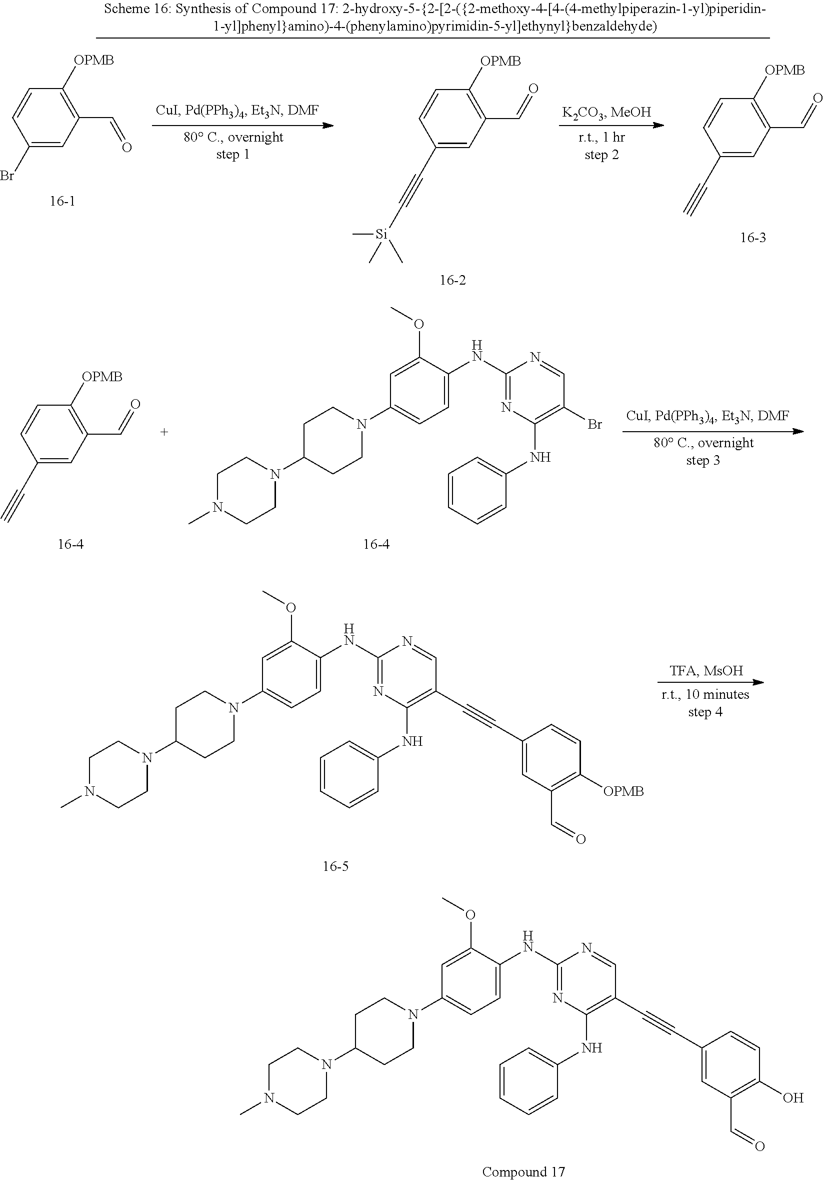

### Step 1: Synthesis of {2-[3-(1,3-dioxolan-2-yl)-4-[(4-methoxyphenyl)methoxy]phenyl]ethynyl}trimethylsilane

2-{5-bromo-2-[(4-methoxyphenyl)methoxy]phenyl}-1,3-dioxolane (500 mg, 1.369 mmol, 1 equiv), a stir bar and DMF (5 mL) were added to a 50-mL round-bottom flask and stirred until homogeneous. trimethylsilylacetylene (403.40 mg, 4.107 mmol, 3 equiv), CuI (26.07 mg, 0.137 mmol, 0.1 equiv), Pd(PPh₃)₄(158.20 mg, 0.137 mmol, 0.1 equiv) and Et₃N (415.61 mg, 4.107 mmol, 3 equiv) were added to the reaction flask at rt. The resulting mixture was stirred at 80° C. for overnight. The crude product was purified by reverse phase flash with the following conditions (0.05% NH₄HCO₃:CH₃CN) to afford {2-[3-(1,3-dioxolan-2-yl)-4-[(4-methoxyphenyl)methoxy]phenyl]ethynyl}trimethylsilane (400 mg, 76.38%) as a light yellow oil. MS (ESI) calculated for C₂₂H₂₆O₄Si, 382.16 m/z, found 383.15 [M+H]⁺.

### Step 2: Synthesis of 2-{5-ethynyl-2-[(4-methoxyphenyl)methoxy]phenyl}-1,3-dioxolane

{2-[3-(1,3-dioxolan-2-yl)-4-[(4-methoxyphenyl)methoxy]phenyl]ethynyl}trimethylsilane (400 mg, 1.046 mmol, 1 equiv), a stir bar and MeOH (4 mL, 98.795 mmol, 94.48 equiv) were added to a 50-mL round-bottom flask and stirred until homogeneous. K₂CO₃ (433.55 mg, 3.138 mmol, 3 equiv) was added to the reaction flask at rt. The resulting mixture was stirred at rt for 1 h. The resulting mixture was concentrated under vacuum to afford 2-{5-ethynyl-2-[(4-methoxyphenyl)methoxy]phenyl}-1,3-dioxolane (220 mg, 67.79%) as a light yellow oil. MS (ESI) calculated for C₁₉H₁₈04, 310.12 m/z, found 311.15 [M+H]⁺.

### Step 3: Synthesis of 5-{2-[3-(1,3-dioxolan-2-yl)-4-[(4-methoxyphenyl)methoxy]phenyl]ethynyl}-N2-{2-methoxy-4-[4-(4-methylpiperazin-1-yl)piperidin-1-yl]phenyl}-N4-phenylpyrimidine-2,4-diamine

2-{5-ethynyl-2-[(4-methoxyphenyl)methoxy]phenyl}-1,3-dioxolane (150 mg, 0.483 mmol, 1 equiv), a stir bar and DMF (5 mL) were added to a 50-mL round-bottom flask and stirred until homogeneous. 5-bromo-N2-{2-methoxy-4-[4-(4-methylpiperazin-1-yl)piperidin-1-yl]phenyl}-N4-phenylpyrimidine-2,4-diamine (267.05 mg, 0.483 mmol, 1 equiv), CuI (9.20 mg, 0.048 mmol, 0.1 equiv), Pd(PPh₃)₄(55.85 mg, 0.048 mmol, 0.1 equiv) and Cs₂CO₃ (472.43 mg, 1.449 mmol, 3 equiv) were added to the reaction flask at rt. The resulting mixture was stirred at 80° C. for overnight. The crude product was purified by reverse phase flash with the following conditions (0.05 NH₄HCO₃:CH₃CN) to afford 5-{2-[3-(1,3-dioxolan-2-yl)-4-[(4-methoxyphenyl)methoxy]phenyl]ethynyl}-N2-{2-methoxy-4-[4-(4-methylpiperazin-1-yl)piperidin-1-yl]phenyl}-N4-phenylpyrimidine-2,4-diamine (100 mg, 26.46%) as a light yellow oil. MS (ESI) calculated for C₄₆H₅₁N₇O₅, 781.40 m/z, found 782.35 [M+H]⁺.

### Step 4: Synthesis of 2-hydroxy-S-{2-[2-({2-methoxy-4-[4-(4-methylpiperazin-1-yl)piperidin-1-yl]phenyl}amino)-4-(phenylamino)pyrimidin-S-yl]ethynyl}benzaldehyde)

5-{2-[3-(1,3-dioxolan-2-yl)-4-[(4-methoxyphenyl)methoxy]phenyl]ethynyl}-N2-{2-methoxy-4-[4-(4-methylpiperazin-1-yl)piperidin-1-yl]phenyl}-N4-phenylpyrimidine-2,4-diamine (140 mg, 0.179 mmol, 1 equiv), a stir bar and TFA (2 mL) were added to a 50-mL round-bottom flask and stirred until homogeneous. Methanesulfonic acid (0.5 mL) was added to the reaction flask at rt. The resulting mixture was stirred at rt for 10 min. The residue was diluted with water, then adjusted to pH 6-7 with NaHCO₃, dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The crude product was then purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 57% B in 9 min, 57% B; Wave Length: 254/220 nm; RT1(min): 8.9; Number Of Runs. After lyophilization, it was afforded the 2-hydroxy-S-{2-[2-({2-methoxy-4-[4-(4-methylpiperazin-1-yl)piperidin-1-yl]phenyl}amino)-4-(phenylamino)pyrimidin-S-yl]ethynyl}benzaldehyde) (22.2 mg, 20.10%) as a yellow solid. MS (ESI) calculated for C₃₆H₃₉N₇O₃, 617.31 m/z, found 618.40 [M+H]⁺.¹H NMR (400 MHz, DMSO-d₆) δ (ppm): 10.95 (s, 1H), 10.20 (s, 1H), 8.65-8.74 (m, 1H), 7.93-8.01 (m, 1H), 7.63 (s, 1H), 7.45-7.55 (m, 4H), 7.28-7.45 (m, 3H), 6.86-6.92 (d, J=8.7 Hz, 1H), 6.73-6.77 (m, 1H), 6.57-6.63 (m, 1H), 6.34-6.41 (m, 1H), 3.81 (s, 3H), 3.58-3.67 (m, 2H), 2.54-2.65 (m, 2H), 2.50-2.52 (m, 5H), 2.22-2.35 (m, 4H), 2.14 (s, 3H), 1.80-1.89 (m, 2H), 1.44-1.58 (m, 2H).

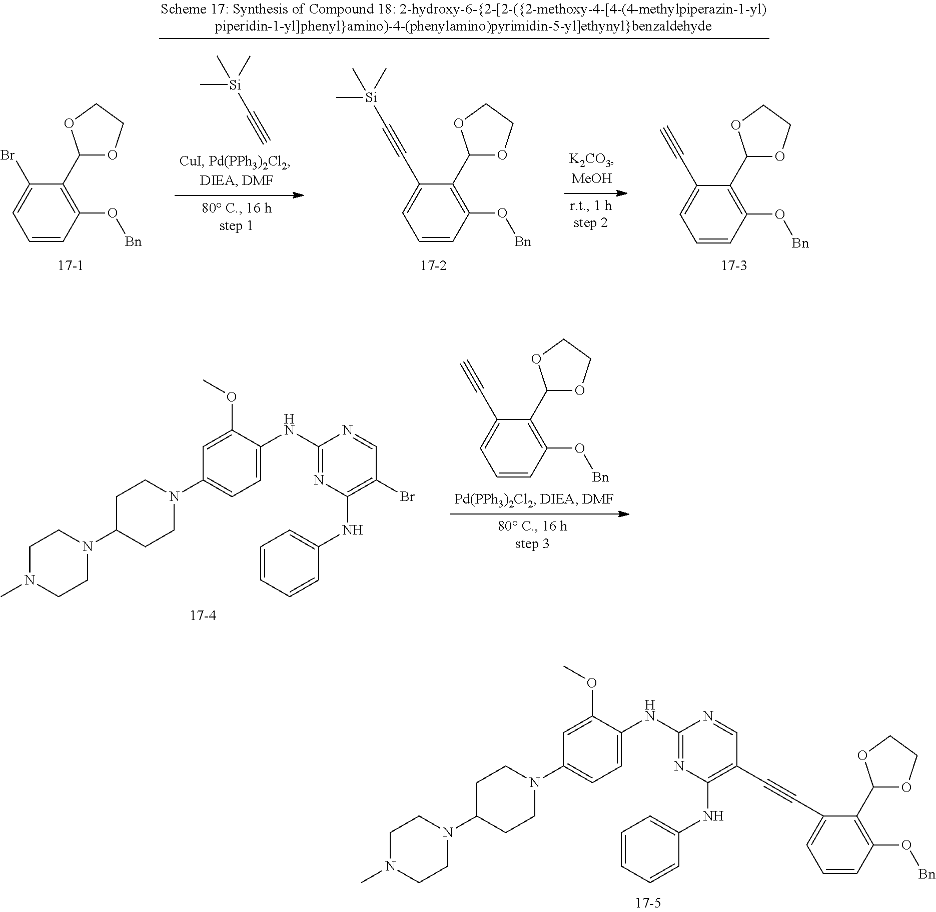

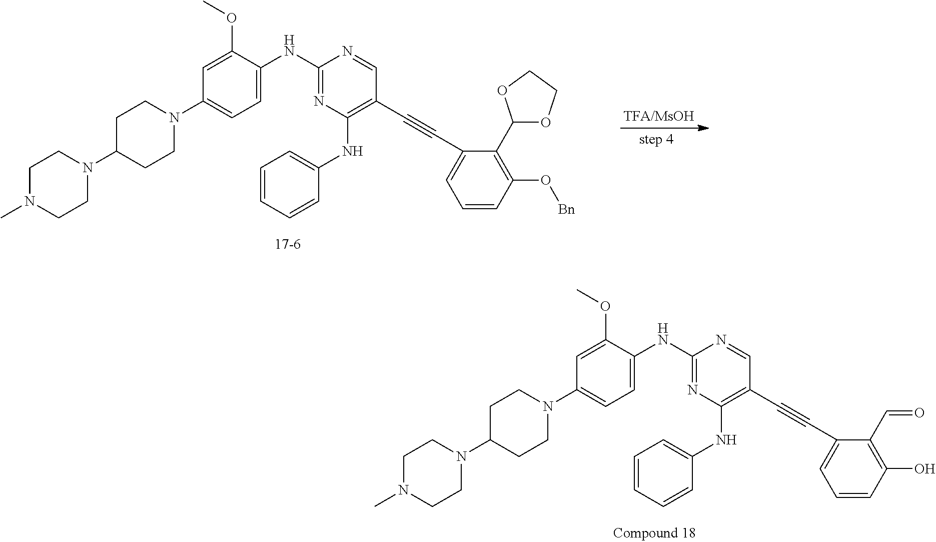 [SMILES: COc1cc(N2CCC(N3CCN(C)CC3)CC2)ccc1Nc1ncc(C#Cc2cccc(OCc3ccccc3)c2C2OCCO2)c(Nc2ccccc2)n1 ; COc1cc(N2CCC(N3CCN(C)CC3)CC2)ccc1Nc1ncc(C#Cc2cccc(O)c2C=O)c(Nc2ccccc2)n1]

### Step 1: Synthesis of {2-[3-(benzyloxy)-2-(1,3-dioxolan-2-yl)phenyl]ethynyl}trimethylsilane

To a suspension of 2-[2-(benzyloxy)-6-bromophenyl]-1,3-dioxolane (3 g, 8.950 mmol, 1 equiv), CuI (0.09 g, 0.448 mmol, 0.05 equiv) and Pd(PPh₃)₂C₁₂ (0.31 g, 0.448 mmol, 0.05 equiv) in DMF (10 mL) was added DIEA (2.31 g, 17.900 mmol, 2 equiv) and trimethylsilylacetylene (2.64 g, 26.850 mmol, 3 equiv) dropwise at rt under nitrogen atmosphere. The obtained solution was stirred for 16 h at 80° C. giving a dark brown solution.

After cooled down to rt, the reaction was quenched by the addition of NH₄Cl aq. (10 mL). The resulting mixture was extracted with Ethyl Acetate (40 mL×3). The combined organic layers were washed with water (40 mL×3), dried over anhydrous Na₂SO₄. After filtration, the filtrate was concentrated under reduced pressure. The residue was purified by reverse flash chromatography with the following conditions: column, C18 silica gel; mobile phase, ACN in water, 10% to 65% gradient in 60 min; detector, UV 254 nm giving {2-[3-(benzyloxy)-2-(1,3-dioxolan-2-yl)phenyl]ethynyl}trimethylsilane (1.5 g, 47.54% yield) as a light brown solid. LC/MS: MS (ESI) calculated for C₂₁H₂₄O₃Si: 352.15. Found: 353.15 [M+H]⁺.

### Step 2: Synthesis of 2-[2-(benzyloxy)-6-ethynylphenyl]-1,3-dioxolane

To a light yellow solution of 2-[3-(benzyloxy)-2-(1,3-dioxolan-2-yl)phenyl]ethynyltrimethylsilane (0.5 g, 1.418 mmol, 1 equiv) in MeOH (10 mL), K₂CO₃ (0.59 g, 4.254 mmol, 3 equiv) was added at 0° C. The reaction was quenched by the addition of NH₄Cl aq. (10 mL). The resulting mixture was extracted with Ethyl Acetate (20 mL×3). The combined organic layers were washed with water (20 mL×3), dried over anhydrous Na₂SO₄. After filtration, the filtrate was concentrated under reduced pressure giving 2-[2-(benzyloxy)-6-ethynylphenyl]-1,3-dioxolane (0.35 g, 88.02%) as a brown oil. LC/MS: mass calculated for C18H₁₆03: 280.11, found: 281.15 [M+H]⁺.

### Step 3: Synthesis of 5-{2-[3-(benzyloxy)-2-(1,3-dioxolan-2-yl)phenyl]ethynyl}-N2-{2-methoxy-4-[4-(4-methylpiperazin-1-yl)piperidin-1-yl]phenyl}-N4-phenylpyrimidine-2,4-diamine

To a suspension of 2-[2-(benzyloxy)-6-ethynylphenyl]-1,3-dioxolane (0.8 g, 2.854 mmol, 1 equiv), CuI (0.03 g, 0.143 mmol, 0.05 equiv) and Pd(PPh₃)₂C₁₂ (0.10 g, 0.143 mmol, 0.05 equiv) in DMF (8 mL) was added 5-bromo-N2-{2-methoxy-4-[4-(4-methylpiperazin-1-yl)piperidin-1-yl]phenyl}-N4-phenylpyrimidine-2,4-diamine (1.73 g, 3.139 mmol, 1.1 equiv) and DIEA (0.74 g, 5.708 mmol, 2 equiv) dropwise at rt under nitrogen atmosphere. The obtained solution was stirred for 16 h at 80° C. giving a brown solution. After cooled down to rt, the reaction was quenched by the addition of NH₄Cl aq. (10 mL). The resulting mixture was extracted with Ethyl Acetate (40 mL×3). The combined organic layers were washed with water (40 mL×3), dried over anhydrous Na₂SO₄. After filtration, the filtrate was concentrated under reduced pressure. The residue was purified by reverse flash chromatography with the following conditions: column, C18 silica gel; mobile phase, ACN in water, 10% to 65% gradient in 60 min; detector, UV 254 nm giving 5-{2-[3-(benzyloxy)-2-(1,3-dioxolan-2-yl)phenyl]ethynyl}-N2-{2-methoxy-4-[4-(4-methylpiperazin-1-yl)piperidin-1-yl]phenyl}-N4-phenylpyrimidine-2,4-diamine (0.7 g, 32.62%) yield) as a light brown solid. LC/MS: mass calculated for C₄₅H₄₉N₇O₄: 751.38, found: 752.20 [M+H]⁺.

### Step 4: Synthesis of 2-hydroxy-6-{2-[2-({2-methoxy-4-[4-(4-methylpiperazin-1-yl)piperidin-1-yl]phenyl}amino)-4-(phenylamino)pyrimidin-S-yl]ethynyl}benzaldehyde

Into a colorless solution of TFA/MsOH (2 mL/0.4 mL), 5-{2-[3-(benzyloxy)-2-(1,3-dioxolan-2-yl)phenyl]ethynyl}-N2-{2-methoxy-4-[4-(4-methylpiperazin-1-yl)piperidin-1-yl]phenyl}-N4-phenylpyrimidine-2,4-diamine (150 mg, 0.199 mmol, 1 equiv) was added at rt under stirring giving a brown solution. The obtained solution was stirred for 1 h at rt. The residue was neutralized to pH=7 with NaHCO₃aq. at 0° C. The resulting mixture was extracted with Ethyl Acetate (3×50 mL). The combined organic layers were washed with water (3×50 mL), dried over anhydrous Na₂SO₄. After filtration, the filtrate was concentrated under reduced pressure giving a brown solid. The residue was submitted to Prep-HPLC with following conditions: Column 30*150 mm 5 μm, n; Mobile Phase A: Water(0.1% FA), Mobile Phase B: ACN; Flow rate: 60 mL/min; Gradient: 40% B to 60% B in 8 min, 60% B; Wave Length: 254/220 nm. After lyophilization, it provided 2-hydroxy-6-{2-[2-({2-methoxy-4-[4-(4-methylpiperazin-1-yl)piperidin-1-yl]phenyl}amino)-4-(phenylamino)pyrimidin-S-yl]ethynyl}benzaldehyde (9.9 mg 7.97% yield). LC/MS: mass calculated for C₃₆H₃₉N₇O₃: 617.31, found: 618.40 [M+H]⁺. LC/MS: mass calculated for C₃₆H₃₉N₇O₃: 617.31, found: 618.35 [M+H]⁺. ¹H NMR (300 MHz, DMSO-d₆) δ ppm: 11.23 (s, 1H), 10.51 (s, 1H), 9.24 (s, 1H), 8.61-8.90 (m, 1H), 8.24-8.39 (s, 1H), 7.73-7.99 (m, 2H), 7.41-7.69 (m, 2H), 7.22-7.40 (m, 2H), 7.09-7.19 (m, 2H), 6.93-7.07 (m, 1H), 6.71 (s, 1H), 6.53-6.56 (m, 1H), 3.79-3.85 (m, 9H), 2.98-3.00 (m, 4H), 2.77 (s, 6H), 2.00-2.05 (m, 2H), 1.62-1.65 (m, 2H).

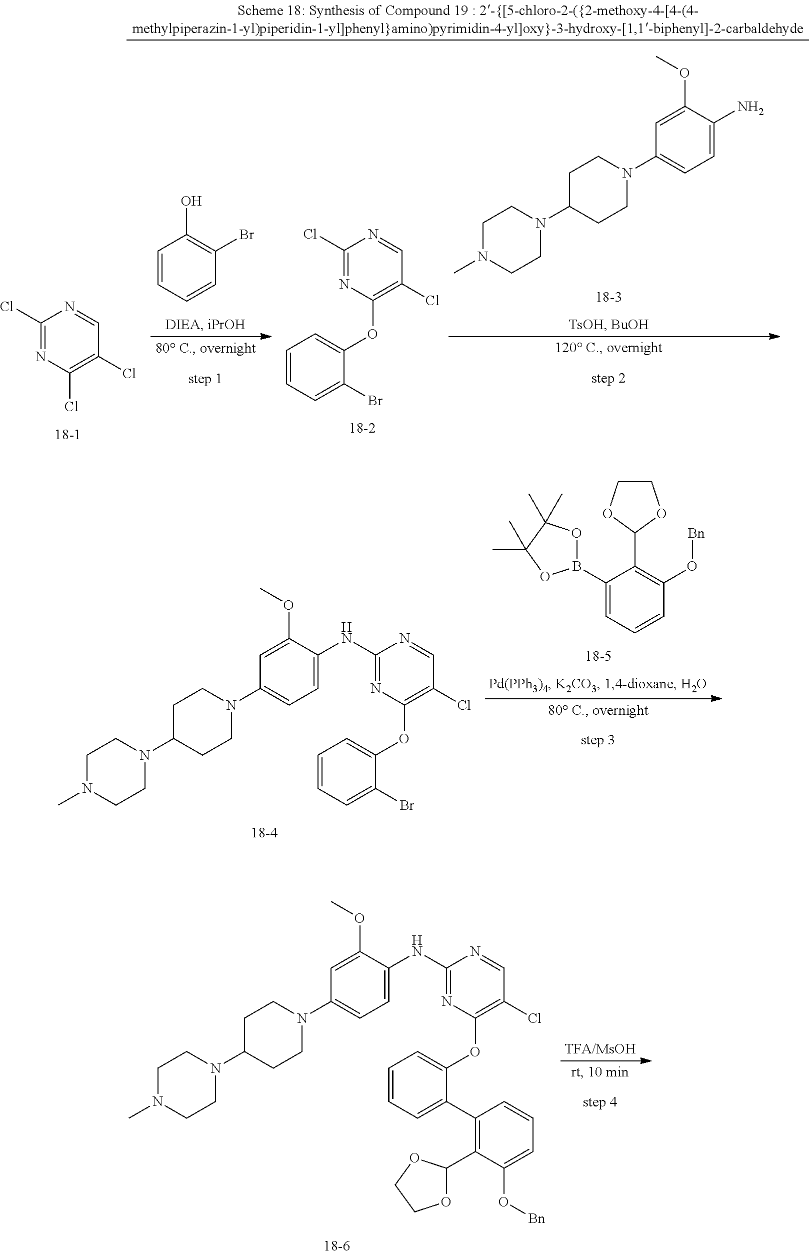

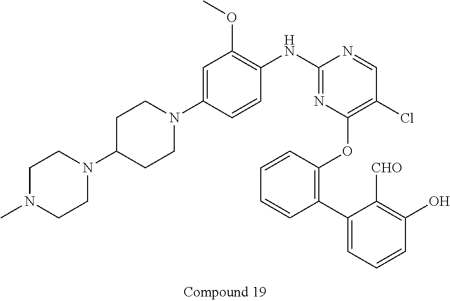 [SMILES: COc1cc(N2CCC(N3CCN(C)CC3)CC2)ccc1Nc1ncc(Cl)c(Oc2ccccc2-c2cccc(O)c2C=O)n1]

### Step 1: Synthesis of 4-(2-bromophenoxy)-2,5-dichloropyrimidine

To a solution of 2,4,5-trichloropyrimidine (20 g, 109.039 mmol) in 2-Propanol (200 mL) was added 2-bromophenol (20.75 g, 119.943 mmol), DIEA (21.14 g, 163.559 mmol). The resulting mixture was maintained under nitrogen and stirred at 90° C. for overnight. LCMS showed the desired product was generated. The reaction was concentrated. The residue obtained was purified by silica gel chromatography (0-20% EtOAc/petroleum ether) to afford 4-(2-bromophenoxy)-2,5-dichloropyrimidine as a yellow solid (27.73 g, 80.00% yield). MS (ESI) calculated for C₁₀H₅BrCl₂N₂O: 317.90 m/z, found: 318.10 [M+H]⁺.

### Step 2: Synthesis of 4-(2-bromophenoxy)-S-chloro-N-(2-methoxy-4-(4-(4-methylpiperazin-1-yl)piperidin-1-yl)phenyl)pyrimidin-2-amine

To a solution of 4-(2-bromophenoxy)-2,5-dichloropyrimidine (4 g, 12.618 mmol) in 2-ethoxyethan-1-ol (40 mL) was added 2-methoxy-4-(4-(4-methylpiperazin-1-yl)piperidin-1-yl) aniline (3.84 g, 12.618 mmol), HCl(g) in MeOH (12 mL). The resulting mixture was maintained under nitrogen and stirred at 120° C. for overnight. LCMS showed the desired product was generated. After cooling down to rt, the reaction was quenched with H₂O (100 mL). The pH value of the aqueous phase was adjusted to 7-8 using a NaHCO₃ solution. The resulting mixture was extracted with EtOAc (3×100 mL). The organic layers were combined, dried over Na₂SO₄, filtered and concentrated. It was afforded the 4-(2-bromophenoxy)-S-chloro-N-(2-methoxy-4-(4-(4-methylpiperazin-1-yl)piperidin-1-yl)phenyl)pyrimidin-2-amine as a yellow solid (3.2 g, 43.27% yield). MS (ESI) calculated for C₂₇H₃₂BrClN₆O_{2-: 586.15} m/z, found: 587.10 [M+H]⁺.

### Step 3: Synthesis of 4-{[3′-(benzyloxy)-2′-(1,3-dioxolan-2-yl)-[1,1′-biphenyl]-2-yl]oxy}-S-chloro-N-{2-methoxy-4-[4-(4-methylpiperazin-1-yl)piperidin-1-yl]phenyl}pyrimidin-2-amine

4-(2-bromophenoxy)-S-chloro-N-(2-methoxy-4-(4-(4-methylpiperazin-1-yl)piperidin-1-yl)phenyl)pyrimidin -2-amine (150 mg, 0.255 mmol, 1 equiv), a stir bar and dioxane (2 mL) and H₂O (0.5 mL) were added to a 50-mL round-bottom flask and stirred until homogeneous. 2-[3-(benzyloxy)-2-(1,3-dioxolan-2-yl)phenyl]-4,4,5,5-tetramethyl-1,3,2-dioxaborolane (117.03 mg, 0.306 mmol, 1.2 equiv), Pd(PPh₃)₄(29.48 mg, 0.026 mmol, 0.1 equiv) and K₂CO₃ (88.15 mg, 0.637 mmol, 2.5 equiv) were added to the reaction flask at rt. The resulting mixture was stirred at 60° c. for overnight under N₂. The reaction mixture was then treated with H₂O (30 mL), dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The crude product was purified by reverse phase flash chromatography with the following conditions (0.05% NH₄HCO₃) to afford 4-{[3′-(benzyloxy)-2′-(1,3-dioxolan-2-yl)-[1,1′-biphenyl]-2-yl]oxy}-S-chloro-N-{2-methoxy-4-[4-(4-methylpiperazin-1-yl)piperidin-1-yl]phenyl}pyrimidin-2-amine (100 mg, 51.35%) as a yellow solid. MS (ESI) calculated for C₄₃H₄₇ClN₆O₅, 762.33 m/z, found 763.20 [M+H]⁺.

### Step 4: Synthesis of 2′-{[5-chloro-2-({2-methoxy-4-[4-(4-methylpiperazin-1-yl)piperidin-1-yl]phenyl} amino)pyrimidin-4-yl]oxy}-3-hydroxy-[1,1′-biphenyl]-2-carbaldehyde

4-{[3′-(benzyloxy)-2′-(1,3-dioxolan-2-yl)-[1,1′-biphenyl]-2-yl]oxy}-S-chloro-N-{2-methoxy-4-[4-(4-methylpiperazin-1-yl)piperidin-1-yl]phenyl}pyrimidin-2-amine (100 mg, 0.131 mmol, 1 equiv), a stir bar and TFA (2 mL, 26.926 mmol, 205.54 equiv) were added to a 50-mL round-bottom flask and stirred until homogeneous. Methanesulfonic acid (0.5 mL, 0.005 mmol, 0.04 equiv) was added to the reaction flask at rt chromatography. The resulting mixture was stirred at rt for 10 min. The residue was diluted with water, then adjusted to pH 6-7 with NaHCO₃, dropwise over 10 min, extracted with DCM (30 mL×2), and the combined extracts washed with brine (10 mL), dried over anhydrous Na₂SO₄, filtered, and concentrated to dryness in vacuo to give a nearly yellow viscous oil. The crude product was then purified by Prep-HPLC with the following conditions: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 25% B to 55% B in 9 min, 55% B; Wave Length: 254 nm; RTT(min): 7; Number Of Runs. After lyophilization, it was afforded the 2′-{[5-chloro-2-({2-methoxy-4-[4-(4-methylpiperazin-1-yl)piperidin-1-yl]phenyl}amino)pyrimidin-4-yl]oxy}-3-hydroxy-[1,1′-biphenyl]-2-carbaldehyde (10.8 mg, 13.03%) as a light yellow solid. MS (ESI) calculated for C₃₄H₃₇ClN₆O₄, 628.26 m/z, found 629.35 [M+H]⁺.¹H NMR (400 MHz, DMSO-d₆) δ (ppm): 11.44 (s, 1H), 9.68 (s, 1H), 8.11 (s, 1H), 8.02 (s, 1H), 7.54-7.64 (m, 1H), 7.44-7.51 (m, 3H), 7.38-7.44 (m, 1H), 7.02-7.09 (m, 1H), 6.91 (d, J=8.4 Hz, 1H), 6.64-6.71 (m, 1H), 6.48-6.53 (m, 1H), 6.16-6.21 (m, 1H), 3.71 (s, 3H), 3.59-3.67 (m, 2H), 2.56-2.67 (m, 3H), 2.49-2.50 (m, 3H), 2.22-2.34 (m, 5H), 2.13 (s, 3H), 1.78-1.86 (m, 2H), 1.41-1.55 (m, 2H).

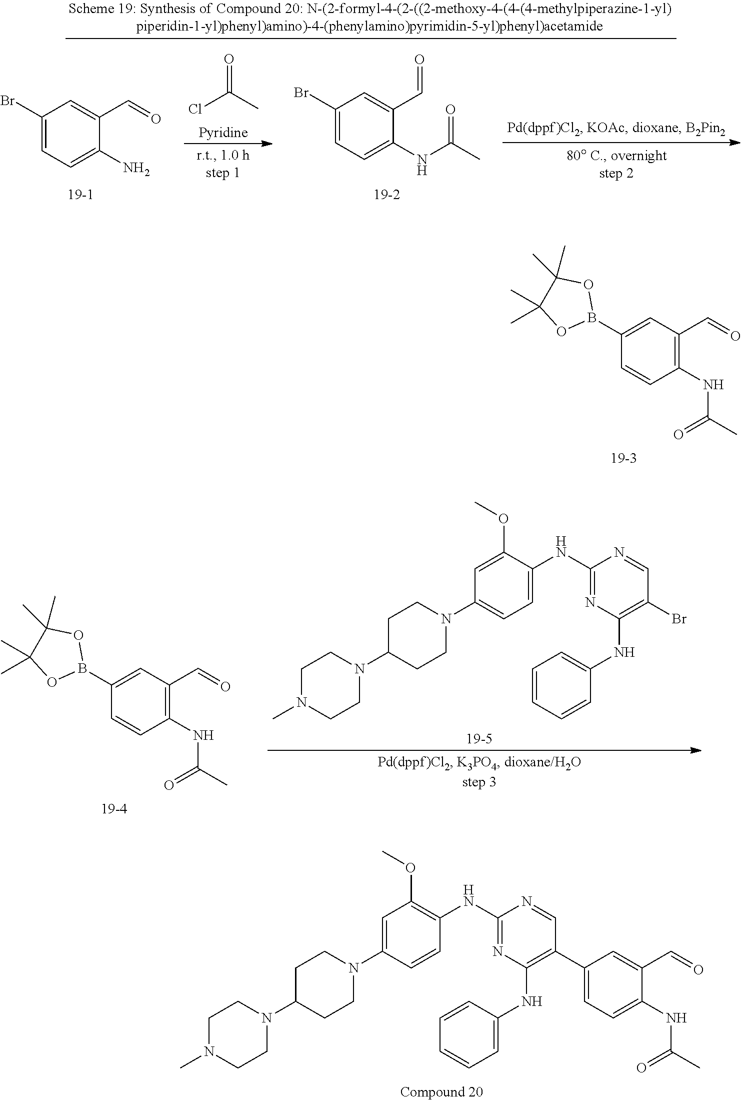

### Step 1: Synthesis of N-(4-bromo-2-formylphenyl)acetamide

A solution of 2-amino-S-bromobenzaldehyde (1.5 g, 7.499 mmol, 1 equiv) in DCM (50 mL) was treated with TEA (3.04 g, 29.996 mmol, 4 equiv) at 0° C. under nitrogen atmosphere followed by the addition of acetyl chloride (2.35 g, 29.996 mmol, 4 equiv) dropwise at 0° C. The resulting mixture was stirred for 3 h at room temperature under N₂ atmosphere. The reaction was quenched with H₂O (100 ml). The resulting mixture was extracted with DCM (100 ml×3). The combined organic layers were washed with NaCl/H₂O (50 ml), dried over anhydrous Na₂SO₄. After filtration, the filtrate was concentrated under reduced pressure. The residue was purified by silica gel column chromatography, eluted with Ethyl Acetate/Petroleum Ether (0˜20%) to afford N-(4-bromo-2-formylphenyl)acetamide (960 mg, 52.89%) as a white solid. MS (ESI) calculated for C₉HsBrNO_{2-, 240.97}, LCMS: m/z 242.05, 244.05 [M+H, M+2+H]⁺.

### Step 2: Synthesis of N-(2-formyl-4-(4,4,5,5-tetramethyl-1,3,2-dioxaborolan-2-yl)phenyl)acetamide

A solution of N-(4-bromo-2-formylphenyl)acetamide (960 mg, 3.966 mmol, 1 equiv), bis(pinacolato)diboron (2014.13 mg, 7.932 mmol, 2 equiv), Pd(dppf)Cl₂ (290.18 mg, 0.397 mmol, 0.1 equiv), KOAc (778.42 mg, 7.932 mmol, 2 equiv) in dioxane (20 mL) was stirred for 5 h at 80° C. under N₂ atmosphere. The mixture was allowed to cool down to room temperature. The reaction was quenched with H₂O (20 ml). The resulting mixture was extracted with Ethyl Acetate (20 ml×3). The combined organic layers were washed with NaCl/H₂O (20 ml), dried over anhydrous Na₂SO₄. After filtration, the filtrate was concentrated under reduced pressure. The residue was purified by silica gel column chromatography, eluted with EA/PE (0˜30%) to afford N-[2-formyl-4-(4,4,5,5-tetramethyl-1,3,2-dioxaborolan-2-yl)phenyl]acetamide (780 mg, 68.02%) as a white solid. MS (ESI) calculated for C₁₅H₂₀BNO₄, 289.15, LCMS: m/z 290.20[M+H]⁺.

### Step 3: Synthesis of N-(2-formyl-4-(2-((2-methoxy-4-(4-(4-methylpiperazin-1-yl)piperidin-1-yl)phenyl)amino)-4-(phenylamino)pyrimidin-S-yl)phenyl)acetamide

The mixture of 5-bromo-N2-{2-methoxy-4-[4-(4-methylpiperazin-1-yl)piperidin-1-yl]phenyl}-N4-phenylpyrimidine-2,4-diamine (150 mg, 0.271 mmol, 1 equiv), N-[2-formyl-4-(4,4,5,5-tetramethyl-1,3,2-dioxaborolan-2-yl)phenyl]acetamide (94.20 mg, 0.325 mmol, 1.2 equiv), Pd(dppf)Cl₂ (9.93 mg, 0.014 mmol, 0.05 equiv), K3PO₄ (144.07 mg, 0.677 mmol, 2.5 equiv) in H₂O (1 mL) and dioxane (4 mL) was stirred for 2 h at 80° C. under N₂ atmosphere. The mixture was allowed to cool down to room temperature. The mixture was purified by reverse-phase flash with the following conditions: Column, Cat No: SO230120˜2, C18, 120 g, 20˜45 μm, 100 Å; mobile phase, CH₃CN:H₂O (0.1% FA)=20% increased to CH₃CN:H₂O (0.1% FA)=40% in 40 min; Detector, UV 220 nm & 254 nm. The solvent was removed under reduced pressure. The crude product was then purified by Prep-HPLC with the following conditions: Column: Sunfire prep C18 column, 30*150 mm, 5 μm; Mobile Phase A: Water(0.1% FA), Mobile Phase B: ACN; Flow rate: 60 mL/min; Gradient: 6% B to 21% B in 8 min, 21% B; Wave Length: 254/220 nm; RT1(min): 8; Number Of Runs: 0. After lyophilization, it was afforded N-{2-formyl-4-[2-({2-methoxy-4-[4-(4-methylpiperazin-1-yl)piperidin-1-yl]phenyl}amino)-4-(phenylamino)pyrimidin-S-yl]phenyl}acetamide (24.2 mg, 13.98%) as a yellow solid. MS (ESI) calculated for C₃₆H₄₂N₈O₃, 634.34, LCMS: m/z 635.35 [M+H]⁺. ¹H NMR (300 MHz, DMSO-d6) δ (ppm): 10.81 (s, 1H), 10.03 (s, 1H), 8.21-8.31 (m, 2H), 7.90-7.98 (m, 2H), 7.62-7.79 (m, 3H), 7.56 (d, J=8.0 Hz, 2H), 7.24 (t, J=7.9 Hz, 2H), 7.01 (t, J=7.3 Hz, 1H), 6.60-6.65 (m, 1H), 6.40-6.50 (m, 1H), 3.81 (s, 3H), 3.60-3.70 (m, 2H), 2.60-2.68 (m, 2H), 2.40-2.45 (m, 4H), 2.32-2.40 (m, 5H), 2.20 (s, 3H), 2.16 (s, 3H), 1.80-1.90 (m, 2H), 1.40-1.70 (m, 2H).

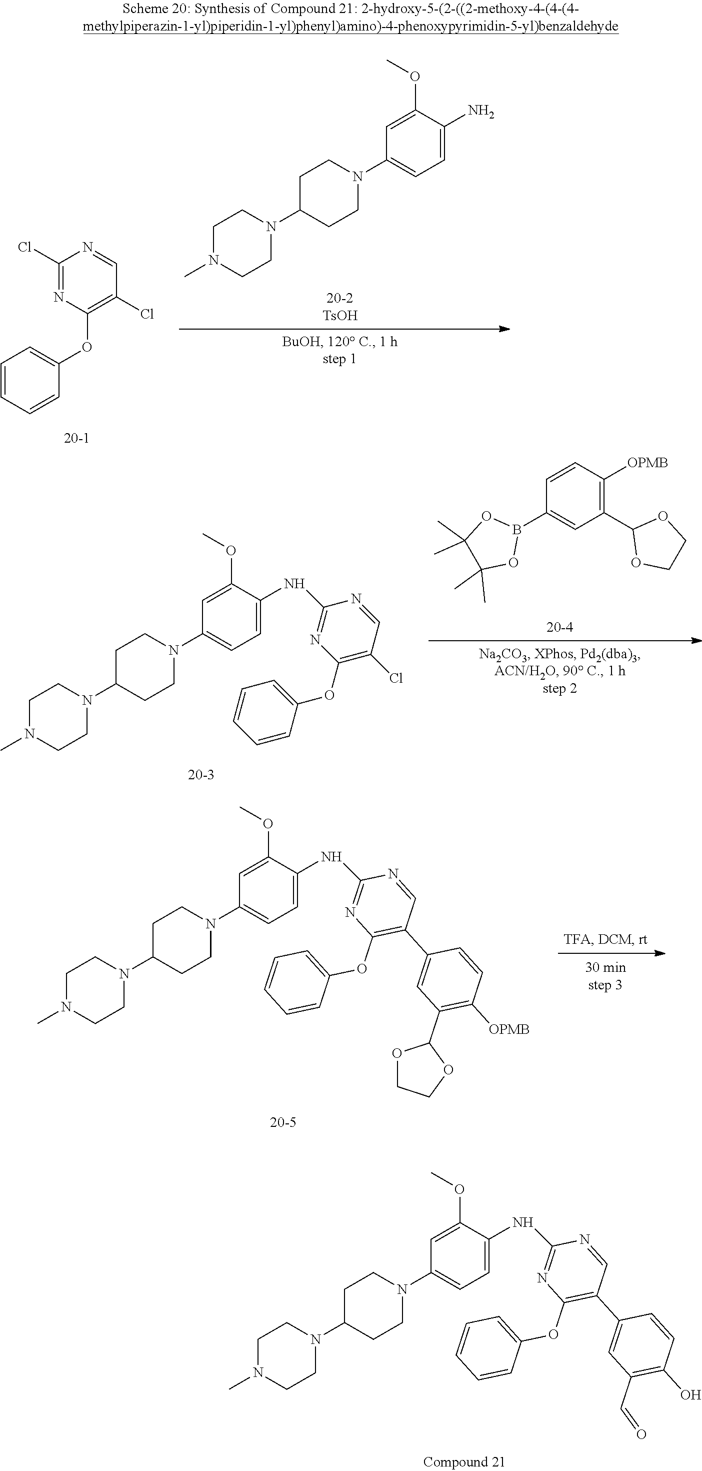

### Step 1: Synthesis of 5-chloro-N-(2-methoxy-4-(4-(4-methylpiperazin-1-yl)piperidin-1-yl)phenyl)-4-phenoxypyrimidin-2-amine

2,5-dichloro-4-phenoxypyrimidine (400 mg, 1.659 mmol, 1 equiv) and n-Butanol (5 mL) were added to the 40 mL reaction vessel, a stir bar, then added 2-methoxy-4-[4-(4-methylpiperazin-1-yl) piperidin-1-yl] aniline (505.14 mg, 1.659 mmol, 1 equiv) and TsOH (428.59 mg, 2.489 mmol, 1.5equiv) to a 100 ml reaction vessel and stirred until homogeneous. The reaction was stirred for additional overnight at 120° C. Nitrogen protection throughout the reaction process. Desired product was detected by LCMS. The reaction was quenched with water, The resulting mixture was extracted with ethyl acetate 100 ml. The combined organic layers dried over anhydrous sodium sulfate. After filtration, the filtrate was concentrated under reduced pressure. The residue was purified by silica gel column chromatography, eluted with MEOH 30% and DCM 70% to afford 5-chloro-N-{2-methoxy-4-[4-(4-methylpiperazin-1-yl) piperidin-1-yl] phenyl}-4-phenoxypyrimidin-2-amine (430 mg, 50.91%) as a yellow solid. MS (ESI) calculated for C₂₇H₃₃ClN₆O_{2-, 508.24} m/z, found 509.20 [M+H]⁺.

### Step 2: Synthesis of 5-(3-(1,3-dioxolan-2-yl)-4-((4-methoxybenzyl)oxy)phenyl)-N-(2-methoxy-4-(4-(4-methylpiperazin-1-yl)piperidin-1-yl)phenyl)-4-phenoxypyrimidin-2-amine

5-chloro-N-{2-methoxy-4-[4-(4-methylpiperazin-1-yl)piperidin-1-yl]phenyl}-4-phenoxypyrimidin-2-amine (120 mg, 0.236 mmol, 1 equiv); H₂O (0.4 mL); ACN (2 mL) were added to a 40 mL reaction vessel along with a stir bar. Na₂CO₃ (49.97 mg, 0.472 mmol, 2equiv); Pd₂(dba)₃ (21.59 mg, 0.024 mmol, 0.1 equiv) and Xphos (11.24 mg, 0.024 mmol, 0.1 equiv) were added to a 40 mL reaction vessel and stirred until homogeneous. Finally, 2-[3-(1,3-dioxolan-2-yl)-4-[(4-methoxyphenyl)methoxy]phenyl]-4,4,5,5-tetramethyl-1,3,2-dioxaborolane (145.79 mg, 0.354 mmol, 1.5 equiv) was added and stirred well. The reaction was stirred overnight at 90° C. Nitrogen protection throughout the reaction process. After cooling down to rt, the reaction mixture was concentrated to dryness to give yellow solid. The crude product (100 mg) was purified by Prep-HPLC with the following to afford 5-[3-(1,3-dioxolan-2-yl)-4-[(4-methoxyphenyl)methoxy]phenyl]-N-{2-methoxy-4-[4-(4-methylpiperazin-1-yl)piperidin-1-yl]phenyl}-4-phenoxypyrimidin-2-amine (60 mg, 33.54%) as a yellow solid.

MS (ESI) calculated for C₄₄H₅₀N₆O₆, 758.38 m/z, found 759.36 [M+H]⁺.

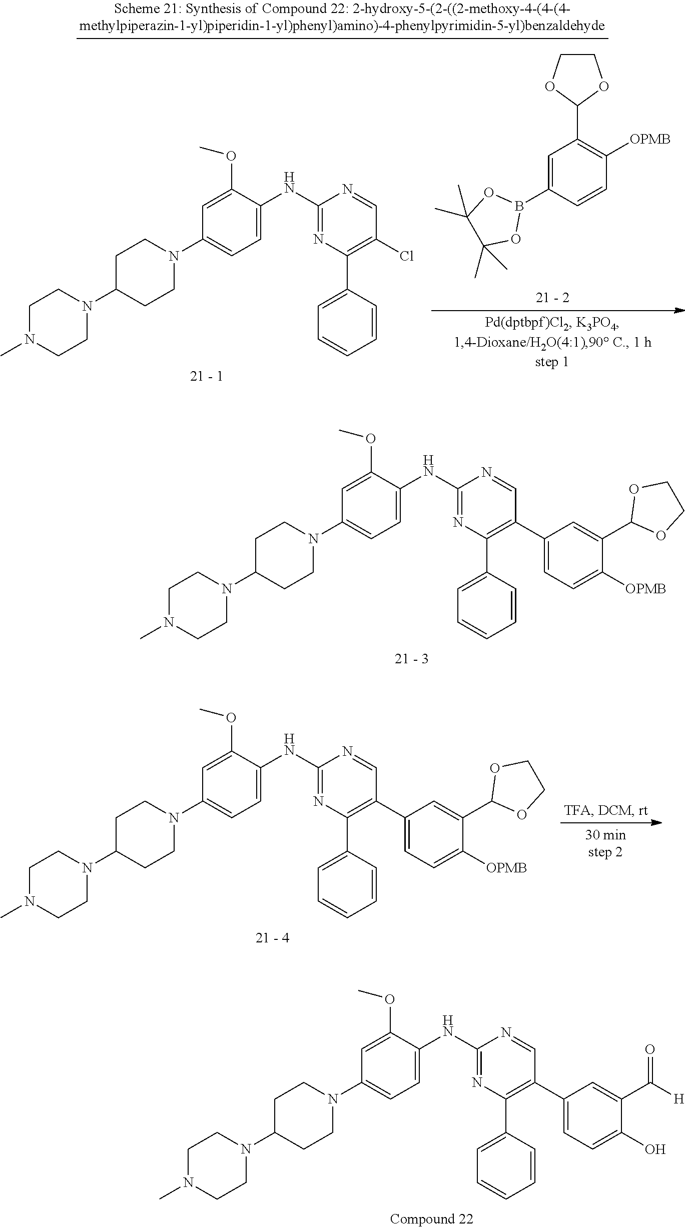

### Step 1: Synthesis of 5-(3-(1,3-dioxolan-2-yl)-4-((4-methoxybenzyl)oxy)phenyl)-N-(2-methoxy-4-(4-(4-methylpiperazin-1-yl)piperidin-1-yl)phenyl)-4-phenylpyrimidin-2-amine

5-chloro-N-{2-methoxy-4-[4-(4-methylpiperazin-1-yl)piperidin-1-yl]phenyl}-4-phenylpyrimidin-2-amine (40 mg, 0.081 mmol, 1 equiv) and H₂O (0.2 mL); dioxane (0.8 mL) were added to the 40 mL reaction vessel, a stir bar, then added K₃PO₄ (34.44 mg, 0.162 mmol, 2 equiv); Pd(DtBPF)Cl₂ (5.29 mg, 0.008 mmol, 0.1 equiv) and 2-[3-(1,3-dioxolan-2-yl)-4-[(4-methoxyphenyl)methoxy]phenyl]-4,4,5,5-tetramethyl-1,3,2-dioxaborolane (50.17 mg, 0.121 mmol, 1.5 equiv) to a 8 mL reaction vessel and stirred until homogeneous. The reaction was stirred for additional overnight at 60° C. Nitrogen protection throughout the reaction process. Desired product was detected by LCMS. The reaction was quenched with water 3 mL. The aqueous layer was extracted with EA 10 ml. The combined organic layers were dried and filtered over anhydrous sodium sulfate. After filtration, the filtrate was concentrated under reduced pressure. The residue was purified by Prep-TLC DCM:MEOH (15:1) to afford 5-[3-(1,3-dioxolan-2-yl)-4-[(4-methoxyphenyl)methoxy]phenyl]-N-{2-methoxy-4-[4-(4-methylpiperazin-1-yl)piperidin-1-yl]phenyl}-4-phenylpyrimidin-2-amine (40 mg, 66.37%) as a yellow solid. MS (ESI) calculated for C₄₄H₅₀N₆O₅, 742.38 m/z, found 743.39 [M+H]⁺.

### Step 2: 2-hydroxy-S-(2-((2-methoxy-4-(4-(4-methylpiperazin-1-yl)piperidin-1-yl)phenyl)amino)-4-phenylpyrimidin-S-yl)benzaldehyde

TFA (0.2 mL) and DCM (1 mL) were added into 40 ml microwave tube, a stir bar, then 5-[3-(1,3-dioxolan-2-yl)-4-[(4-methoxyphenyl)methoxy]phenyl]-N-{2-methoxy-4-[4-(4-methylpiperazin-1-yl)piperidin-1-yl]phenyl}-4-phenylpyrimidin-2-amine (40 mg, 0.054 mmol, 1 equiv) was added to a 40 ml microwave tube and stirred until homogeneous. The reaction was stirred 10 min at rt. Desired product was detected by LCMS. The reaction was quenched with water 3 ml. The aqueous layer was extracted with EA 10 ml. The combined organic layers dried over anhydrous sodium sulfate. After filtration, the filtrate was concentrated under reduced pressure. The residue was purified by silica gel column chromatography, MEOH:DCM=8:1,to afford 2-hydroxy-S-[2-({2-methoxy-4-[4-(4-methylpiperazin-1-yl)piperidin-1-yl]phenyl}amino)-4-phenylpyrimidin-S-yl]benzaldehyde (5.1 mg, 15.65%) as a yellow solid. MS (ESI) calculated for C₃₄H₃₈N₆O₃, 578.30 m/z, found 579.32 [M+H]⁺. 1H NMR (300 MHz, DMSO-d6) δ 10.85-10.70 (m, 1H), 10.22 (s, 1H), 8.38 (s, 1H), 8.16 (s, 1H), 7.78 (d, J=8.7 Hz, 1H), 7.48 (d, J=2.4 Hz, 1H), 7.42-7.27 (m, 5H), 7.20 (dd, J=8.6, 2.5 Hz, 1H), 6.90 (d, J=8.6 Hz, 1H), 6.65 (d, J=2.5 Hz, 1H), 6.51 (dd, J=8.8, 2.5 Hz, 1H), 3.83 (s, 3H), 3.72 (d, J=12.0 Hz, 2H), 2.76-2.56 (m, 11H), 2.42-2.25 (m, 3H), 1.89-1.83 (m, 2H), 1.59-1.49 (m, 2H).

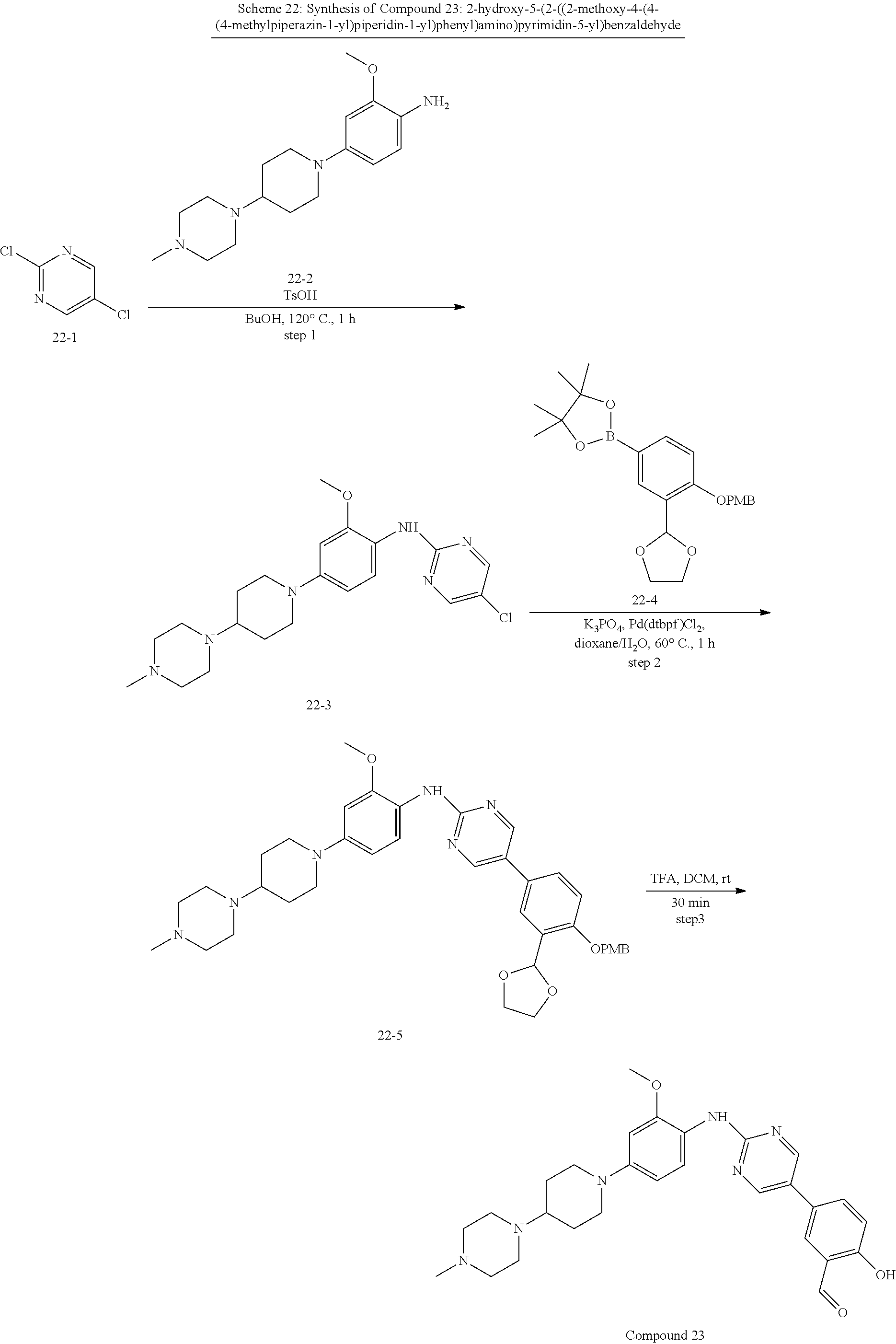

### Step 1: Synthesis of 5-chloro-N-(2-methoxy-4-(4-(4-methylpiperazin-1-yl)piperidin-1-yl)phenyl)pyrimidin-2-amine

2,5-dichloropyrimidine (500 mg, 3.356 mmol, 1 equiv) and t-BuOH (5 mL) were added into 40 ml microwave tube, a stir bar, then and TFA (574.06 mg, 5.034 mmol, 1.5 equiv) and 2-methoxy-4-[4-(4-methylpiperazin-1-yl)piperidin-1-yl]aniline (1021.81 mg, 3.356 mmol, 1 equiv) were added to a 40 ml microwave tube and stirred until homogeneous. The reaction was stirred 1h at 100° C. Nitrogen protection throughout the reaction process. The desired product was detected by LCMS. The reaction was quenched with water 3 ml, the mixture was neutralized to pH 8 with saturated sodium bicarbonate in aqueous solution. The aqueous layer was extracted with EA 20 ml. The combined organic layers dried over anhydrous sodium sulfate. After filtration, the filtrate was concentrated under reduced pressure. The residue was purified by silica gel column chromatography, EA:PE=1:5, to afford 5-chloro-N-{2-methoxy-4-[4-(4-methylpiperazin-1-yl)piperidin-1-yl]phenyl}pyrimidin-2-amine (1 g, 71.46%) as a yellow solid. MS (ESI) calculated for C₂₁H₂₉ClN₆O, 416.21 m/z, found 417.20 [M+H]⁺.

### Step 2: Synthesis of 5-(3-(1,3-dioxolan-2-yl)-4-((4-methoxybenzyl)oxy)phenyl)-N-(2-methoxy-4-(4-(4-methylpiperazin-1-yl)piperidin-1-yl)phenyl)pyrimidin-2-amine

5-chloro-N-{2-methoxy-4-[4-(4-methylpiperazin-1-yl)piperidin-1-yl]phenyl}pyrimidin-2-amine (200 mg, 0.480 mmol, 1 equiv); H₂O (0.02 mL) dioxane (0.8 mL) were added to a 40 mL reaction vessel, Then a stir bar, And then added K3PO₄ (203.64 mg, 0.960 mmol, 2 equiv); Pd(DtBPF)Cl₂ (31.26 mg, 0.048 mmol, 0.1 equiv) to a 40 mL reaction vessel and stirred until homogeneous, Finally added 2-[3-(1,3-dioxolan-2-yl)-4-[(4-methoxyphenyl)methoxy]phenyl]-4,4,5,5-tetramethyl-1,3,2-dioxaborolane (296.65 mg, 0.720 mmol, 1.5 equiv) and stir well. The reaction was stirred overnight at 60° C. Nitrogen protection throughout the reaction process. After cooling down to rt, the reaction mixture was concentrated to dryness to give yellow solid. The crude product (140 mg) was purified by Prep-HPLC with the following to afford 5-[3-(1,3-dioxolan-2-yl)-4-[(4-methoxyphenyl)methoxy]phenyl]-N-{2-methoxy-4-[4-(4-methylpiperazin-1-yl)piperidin-1-yl]phenyl}pyrimidin-2-amine (180 mg, 56.28%) as a yellow solid. MS (ESI) calculated for C₃₈H₄₆N₆O₅, 666.35 m/z, found 667.32 [M+H]⁺.

### Step 3: Synthesis of 2-hydroxy-S-(2-((2-methoxy-4-(4-(4-methylpiperazin-1-yl)piperidin-1-yl)phenyl)amino)pyrimidin-S-yl)benzaldehyde

TFA (0.4 mL, 5.385 mmol) and DCM (2 mL, 31.461 mmol) were added to a 40 ml reaction vessel and stirred until homogeneous. Then, 5-[3-(1,3-dioxolan-2-yl)-4-[(4-methoxyphenyl)methoxy]phenyl]-N-{2-methoxy-4-[4-(4-methylpiperazin-1-yl)piperidin-1-yl]phenyl}pyrimidin-2-amine (120 mg, 0.180 mmol, 1 equiv) was added to the 40 ml reaction vessel, a stir bar, the reaction was stirred 20 min at rt. The resulting mixture was concentrated under reduced pressure. The crude product (120 mg) was purified by Prep-HPLC MEOH:DCM=1:10 to afford 2-hydroxy-S-[2-({2-methoxy-4-[4-(4-methylpiperazin-1-yl)piperidin-1-yl]phenyl}amino)pyrimidin-S-yl]benzaldehyde (7.6 mg, 8.38%) as a yellow solid. MS (ESI) calculated for C₂₈H₃₄N₆O₃, 502.27 m/z, found 503.29[M+H]⁺. 1H NMR (300 MHz, DMSO-d6) δ 10.31 (s, 1H), 8.70-8.59 (m, 2H), 8.15-7.98 (m, 1H), 7.98-7.78 (m, 1H), 7.75-7.62 (m, 2H), 7.14-6.96 (m, 1H), 6.67-6.60 (m, 1H), 6.55-6.45 (m, 1H), 3.80 (s, 3H), 3.69 (d, J=11.4 Hz, 2H), 2.65 (m, 6H), 2.30 (s, 5H), 2.15 (s, 3H), 1.85 (d, J=12.2 Hz, 2H), 1.60-1.43 (m, 2H).

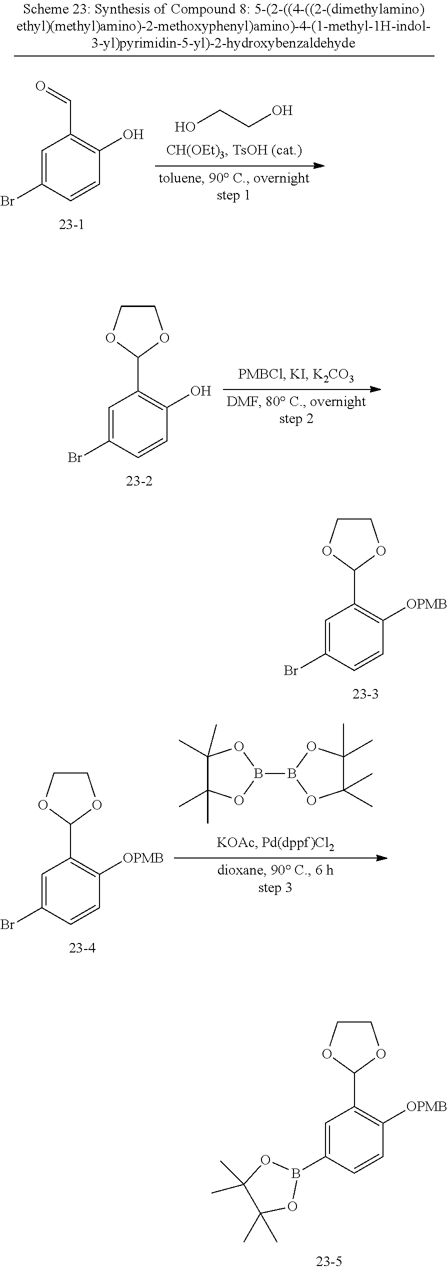

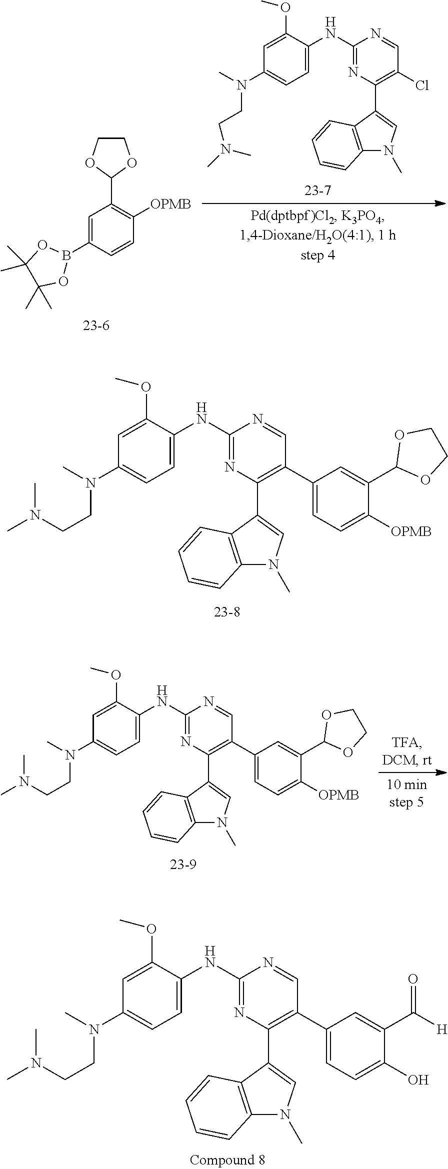

### Step 1: Synthesis of 4-bromo-2-(1,3-dioxolan-2-yl)phenol

3-bromo-6-hydroxycyclohexa-2,4-dien-1-one (5 g, 26.454 mmol, 1 equiv) and Toluene (40 mL) were added to the 100 ml reaction vessel, a stir bar, then added triethyl orthoformate (19.60 g, 132.270 mmol, 5 equiv), ethylene glycol (9.85 g, 158.724 mmol, 6 equiv) and TSOH (0.91 g, 5.291 mmol, 0.2 equiv) to a 100 ml reaction vessel and stirred until homogeneous. The reaction stirred for additional overnight at 90° C. Nitrogen protection throughout the reaction process. Desired product was detected by LCMS. The reaction was quenched with water. The resulting mixture was extracted with ethyl acetate 70 ml. The combined organic layers dried over anhydrous sodium sulfate. After filtration, the filtrate was concentrated under reduced pressure. The residue was purified by silica gel column chromatography, eluted with ethyl acetate 18% and petroleum ether 82% to afford 4-bromo-2-(1,3-dioxolan-2-yl) phenol (6 g, 92.55%) as a yellow solid. MS (ESI) calculated for C₉H₉BrO₃, 245.07 m/z, found 247.70[M+H]⁺.

### Step 2: Synthesis of 2-(5-bromo-2-((4-methoxybenzyl)oxy)phenyl)-1,3-dioxolane

4-bromo-2-(1,3-dioxolan-2-yl) phenol (4 g, 16.322 mmol, 1 equiv) and DMF (30 mL) were added to the 100 ml reaction vessel, a stir bar, then added K₂CO₃ (4.51 g, 32.644 mmol, 2 equiv) and 4-methoxybenzyl chloride (2.56 g, 16.322 mmol, 1 equiv) to a 100 ml reaction vessel and stirred until homogeneous. The reaction stirred for additional overnight at 80° C. Nitrogen protection throughout the reaction process. Desired product was detected by LCMS. The reaction was quenched with water 15 ml. The resulting mixture was extracted with ethyl acetate 60 ml. The combined organic layers were washed with water 40 ml. The combined organic layers dried over anhydrous sodium sulfate. After filtration, the filtrate was concentrated under reduced pressure. The residue was purified by silica gel column chromatography, eluted with ethyl acetate 71% and petroleum ether 29% to afford 2-{5-bromo-2-[(4-methoxyphenyl)methoxy]phenyl}-1,3-dioxolane (4.3 g, 72.13%) as a white solid. MS (ESI) calculated for C₁₇H₁₇BrO₄, 364.03 m/z, found 406.25[M+H]⁺.

### Step 3: Synthesis of 2-(3-(1,3-dioxolan-2-yl)-4-((4-methoxybenzyl)oxy)phenyl)-4,4,5,5-tetramethyl-1,3,2-dioxaborolane

2-(5-bromo-2-((4-methoxybenzyl)oxy)phenyl)-1,3-dioxolane (1 g, 2.738 mmol, 1 equiv) and dioxane (8 mL) were added to the 100 ml reaction vessel, a stir bar, then added KOAc (0.81 g, 8.214 mmol, 3 equiv), Pd(dppf)Cl₂ (0.20 g, 0.274 mmol, 0.1 equiv) and bis(pinacolato)diboron (0.70 g, 2.738 mmol, 1 equiv) to a 100 ml reaction vessel and stirred until homogeneous. The reaction was stirred 6 h at 90° C. Nitrogen protection throughout the reaction process. Desired product was detected by LCMS. The reaction was quenched with water 3 ml. The aqueous layer was extracted with EA 30 ml. The combined organic layers dried over anhydrous sodium sulfate. After filtration, the filtrate was concentrated under reduced pressure. The residue was purified by silica gel column chromatography, eluted with PE:EA(4:1) to afford 2-(3-(1,3-dioxolan-2-yl)-4-((4-methoxybenzyl)oxy)phenyl)-4,4,5,5-tetramethyl-1,3,2-dioxaborolane (1 g, 88.58%) as a yellow oil. MS (ESI) calculated for C₂₃H₂₉BO₆, 412.20 m/z, found 413.15[M+H]⁺.

### Step 4: Synthesis of N1-(5-(3-(1,3-dioxolan-2-yl)-4-((4-methoxybenzyl)oxy)phenyl)-4-(1-methyl-1H-indol-3-yl)pyrimidin-2-yl)-N4-(2-(dimethylamino)ethyl)-2-methoxy-N4-methylbenzene-1,4-diamine

N4-[5-chloro-4-(1-methylindol-3-yl)pyrimidin-2-yl]-N1-[2-(dimethylamino)ethyl]-3-methoxy-N1-methylbenzene-1,4-diamine (120 mg, 0.258 mmol, 1 equiv) and H₂O (0.4 mL), dioxane (2 mL) were added into 8 ml reaction vessel, a stir bar, then K₃PO₄ (109.56 mg, 0.516 mmol, 2 equiv, Pd(DtBPF)Cl₂ (16.82 mg, 0.026 mmol, 0.1 equiv) and 2-[3-(1,3-dioxolan-2-yl)-4-[(4-methoxyphenyl)methoxy]phenyl]-4,4,5,5-tetramethyl-1,3,2-dioxaborolane (159.60 mg, 0.387 mmol, 1.5 equiv) were added to a 40 ml reaction vessel and stirred until homogeneous.

The reaction was stirred overnight at 90° C. Nitrogen protection throughout the reaction process. Desired product was detected by LCMS. After cooling down to r.t, the reaction mixture was concentrated to dryness to give yellow solid. The crude product (140 mg) was purified by silica gel column chromatography, eluted with PE:EA=3:1. to affordN1-[2-(dimethylamino)ethyl]-N4-{5-[3-(1,3-dioxolan-2-yl)-4-[(4-methoxyphenyl)methoxy]-S-(1-methylindol-3-yl)phenyl]pyrimidin-2-yl}-3-methoxy-N1-methylbenzene-1,4-diamine (100 mg, 54.21%) as a yellow solid. MS (ESI) calculated for C₄₂H₄₆N₆O₅, 714.35 m/z, found 715.38[M+H]⁺.

### Step 5: Synthesis of 5-(2-((4-((2-(dimethylamino)ethyl)(methyl)amino)-2-methoxyphenyl)amino)-4-(1-methyl-1H-indol-3-yl)pyrimidin-S-yl)-2-hydroxybenzaldehyde

TFA (1 mL) and DCM (3 mL) were added into 40 ml microwave tube, a stir bar, then N1-[2-(dimethylamino)ethyl]-N4-{5-[3-(1,3-dioxolan-2-yl)-4-[(4-methoxyphenyl)methoxy]-S-(1-methylindol-3-yl)phenyl]pyrimidin-2-yl}-3-methoxy-N1-methylbenzene-1,4-diamine (100 mg, 0.140 mmol, 1 equiv) was added to a 40 ml microwave tube and stirred until homogeneous. The reaction was stirred 10 min at rt. Desired product was detected by LCMS. The reaction was quenched with water 3 ml. The aqueous layer was extracted with EA 10 ml. The combined organic layers dried over anhydrous sodium sulfate. After filtration, the filtrate was concentrated under reduced pressure. The residue was purified by silica gel column chromatography, MEOH:DCM=7:1, to afford 5-{2-[(4-{[2-(dimethylamino)ethyl](methyl)amino}-2-methoxyphenyl)amino]pyrimidin-S-yl}-2-hydroxy-3-(1-methylindol-3-yl)benzaldehyde (5.9 mg, 7.59%) as a yellow solid. MS (ESI) calculated for C₃₂H₃₄N₆O₃, 550.27 m/z, found 551.26 [M+H]⁺. ¹H NMR (300 MHz, DMSO-d6) δ 10.29 (s, 1H), 8.16 (d, J=8.1 Hz, 1H), 8.09 (s, 1H), 7.99 (s, 1H), 7.61 (d, J=2.4 Hz, 1H), 7.53 (d, J=8.7 Hz, 1H), 7.45-7.35 (m, 2H), 7.17 (ddd, J=8.2, 6.9, 1.2 Hz, 1H), 7.06-6.92 (m, 2H), 6.78 (s, 1H), 6.41 (d, J=2.6 Hz, 1H), 6.29 (dd, J=8.8, 2.6 Hz, 1H), 3.80 (s, 3H), 3.63-3.56 (m, 5H), 2.95 (s, 3H), 2.43 (t, J=7.2 Hz, 2H), 2.21 (s, 6H).

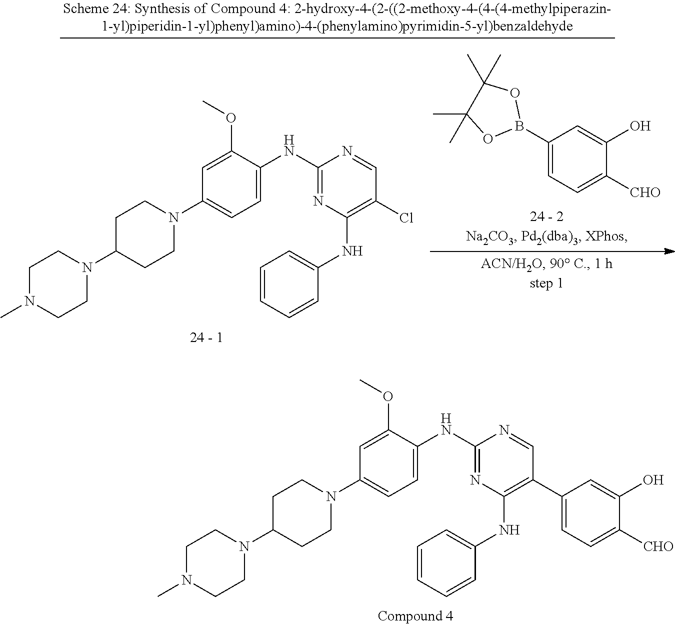

### Step 1: Synthesis of 2-hydroxy-4-(2-((2-methoxy-4-(4-(4-methylpiperazin-1-yl)piperidin-1-yl)phenyl)amino)-4-(phenylamino)pyrimidin-S-yl)benzaldehyde

5-chloro-N2-{2-methoxy-4-[4-(4-methylpiperazin-1-yl)piperidin-1-yl]phenyl}-N4-phenylpyrimidine-2,4-diamine (100 mg, 0.197 mmol, 1 equiv) was added to the 100 ml reaction vessel, a stir bar, 2-hydroxy-4-(4,4,5,5-tetramethyl-1,3,2-dioxaborolan-2-yl)benzaldehyde (73.24 mg, 0.295 mmol, 1.5 equiv), Pd₂(dba)₃ (18.02 mg, 0.020 mmol, 0.1 equiv),XPhos (9.38 mg, 0.020 mmol, 0.1 equiv) Na₂CO₃ (41.72 mg, 0.394 mmol, 2 equiv) and ACN/H₂O(5:1)(10 mL) were added to a 100 mL reaction vessel and stirred until homogeneous. The reaction was stirred overnight at 90° C. Nitrogen protection throughout the reaction process. After cooling down to rt, the reaction mixture was concentrated to dryness to give yellow solid and was purified by preparative HPLC using a XSelect CSH Fluoro Phenyl, 30*150 mm, 5 μm column (eluent: 55% to 65% (v/v) MeOH and H₂O with 10 mmol/L NH₄HCO₃) to afford 2-hydroxy-4-({2-methoxy-4-[4-(4-methylpiperazin-1-yl)piperidin-1-yl]phenyl}amino)-4-(phenylamino)pyrimidin-S-yl]benzaldehyde as a yellow solid. MS(ESI) calculated for C₃₄H₃₉N₇O₃:593.734m/z,found 594.20[M+H]⁺. ¹H NMR (300 MHz, DMSO-d6) δ 10.29 (s, 1H), 8.36 (s, 1H), 7.97 (s, 1H), 7.81 (s, 1H), 7.73 (d, J=8.1 Hz, 1H), 7.66-7.53 (m, 3H), 7.24 (t, J=7.9 Hz, 2H), 7.14-7.08 (m, 1H), 7.11-6.95 (m, 2H), 6.62 (d, J=2.5 Hz, 1H), 6.40 (dd, J=8.9, 2.5 Hz, 1H), 3.80 (s, 3H), 3.69 (d, J=12.0 Hz, 2H), 2.71-2.58 (m, 2H), 2.37-2.23 (m, 8H), 2.15-2.10 (m, 4H), 1.94-1.79 (m, 2H), 1.62-1.44 (m, 2H).

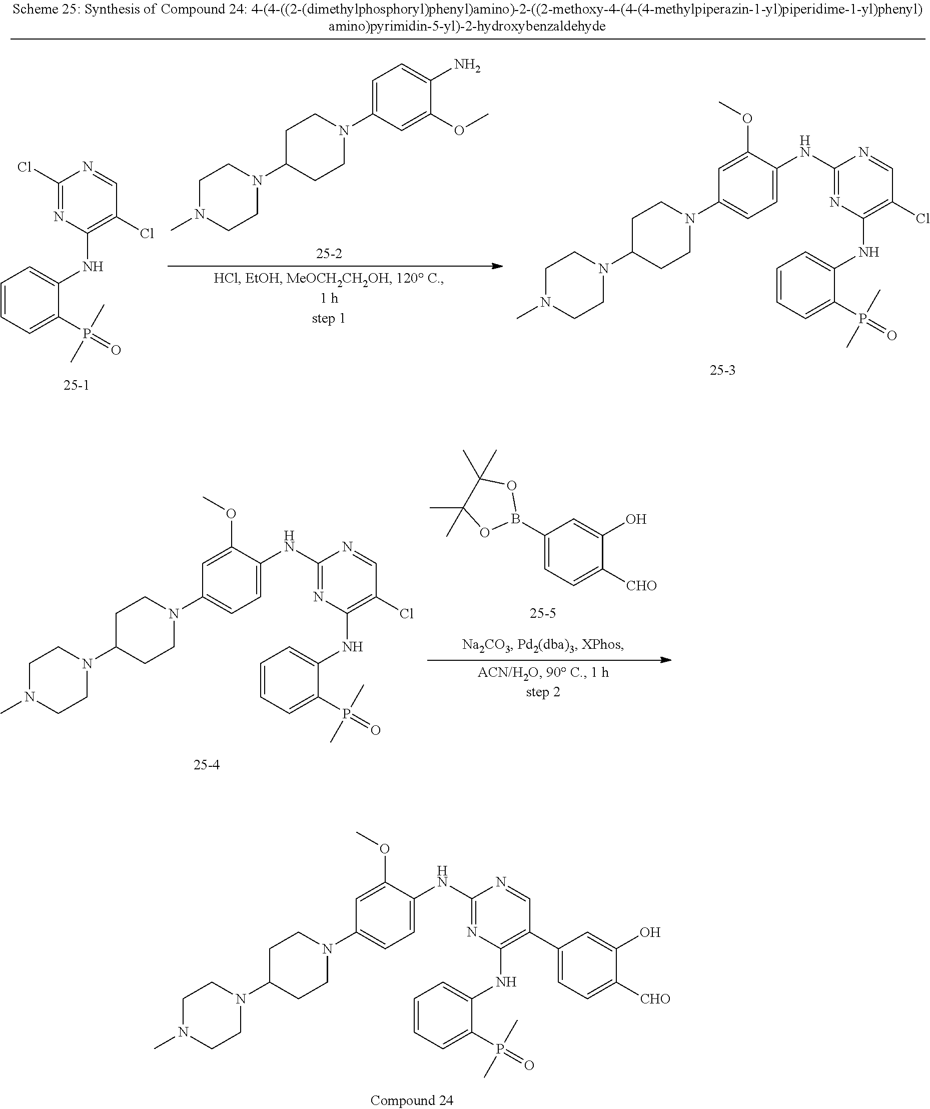

### Step 1: Synthesis of (2-((5-chloro-2-((2-methoxy-4-(4-(4-methylpiperazin-1-yl)piperidin-1-yl)phenyl)amino)pyrimidin-4-yl)amino)phenyl)dimethylphosphine oxide

2,5-dichloro-N-[2-(dimethylphosphoryl)phenyl]pyrimidin-4-amine (100 mg, 0.316 mmol, 1 equiv), a stir bar, 2-methoxy-4-[4-(4-methylpiperazin-1-yl)piperidin-1-yl]aniline (96.30 mg, 0.316 mmol, 1 equiv), Hydrochloric acid methanol solution (28.83 mg, 0.790 mmol, 2.5 equiv) and 2-Ethoxyethanol (4 mL) were added to a 100 mL reaction vessel and stirred until homogeneous. The reaction was stirred overnight at 120° C. Nitrogen protection throughout the reaction process. After cooling down to rt, the reaction mixture was concentrated to dryness to give black solid. The black solid was purified by preparative HPLC using a XSelect CSH Fluoro Phenyl, 30*150 mm, 5 μm column (eluent: 1% to 80% (v/v) MEOH and H₂O with 10 mmol/L NH₄HCO₃) to afford brigatinib (100 mg, 54.12%) as a black solid. MS (ESI), calculated for C₂₉H₃₉ClN₇O₂P:583.26 m/z, found 584.30[M+H]⁺.

### Step 2: Synthesis of 4-(4-((2-(dimethylphosphoryl)phenyl)amino)-2-((2-methoxy-4-(4-(4-methylpiperazin-1-yl)piperidin-1-yl)phenyl)amino)pyrimidin-S-yl)-2-hydroxybenzaldehyde

Brigatinib (100 mg, 0.171 mmol, 1 equiv) and ACN (4 mL) were added to a 100 mL reaction vessel. Then, a stir bar, Na₂CO₃ (36.29 mg, 0.342 mmol, 2 equiv); Pd₂(dba)₃ (15.68 mg, 0.017 mmol, 0.1 equiv) and XPhos (8.16 mg, 0.017 mmol, 0.1 equiv) were added to a 100 mL reaction vessel and stirred until homogeneous, Finally added 2-hydroxy-4-(4,4,5,5-tetramethyl-1,3,2-dioxaborolan-2-yl)benzaldehyde (63.71 mg, 0.257 mmol, 1.5 equiv) and stir well. The reaction was stirred overnight at 120° C. Nitrogen protection throughout the reaction process. After cooling down to rt, the reaction mixture was concentrated to dryness to give yellow solid. The yellow solid was purified by preparative HPLC using a XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 20% B to 40% B in 10 min, 40% B; Wave Length: 254/220 nm; RT1(min): 8.9; Number Of Runs: 0 to afford 4-(4-{[2-(dimethylphosphoryl)phenyl]amino}-2-({2-methoxy-4-[4-(4-methylpiperazin-1-yl)piperidin-1-yl]phenyl}amino)pyrimidin-S-yl)-2-hydroxybenzaldehyde(4 mg, 3.35%) as a yellow solid. ¹H NMR (300 MHz, DMSO-d6) δ 10.49 (s, 1H), 10.24 (s, 1H), 8.37 (s, 1H), 8.02-7.89 (m, 2H), 7.66-7.60 (m, 1H), 7.58-7.52 (m, 1H), 7.52-7.39 (m, 1H), 7.42-7.36 (m, 1H), 7.07-7.00 (m, 2H), 7.00 (s, 1H), 6.47-6.41 (m, 2H), 3.79 (s, 3H), 3.70 (d, J=11.8 Hz, 4H), 2.72-2.59 (m, 4H), 2.31 (s, 5H), 2.15 (s, 3H), 1.86 (d, J=11.7 Hz, 2H), 1.63 (d, J=13.5 Hz, 6H), 1.57-1.47 (m, 2H), 1.24 (s, 1H). MS (ESI), calculated for C₃₆H₄₄N₇O₄P:669.32 m/z, found 670.25[M+H]⁺.

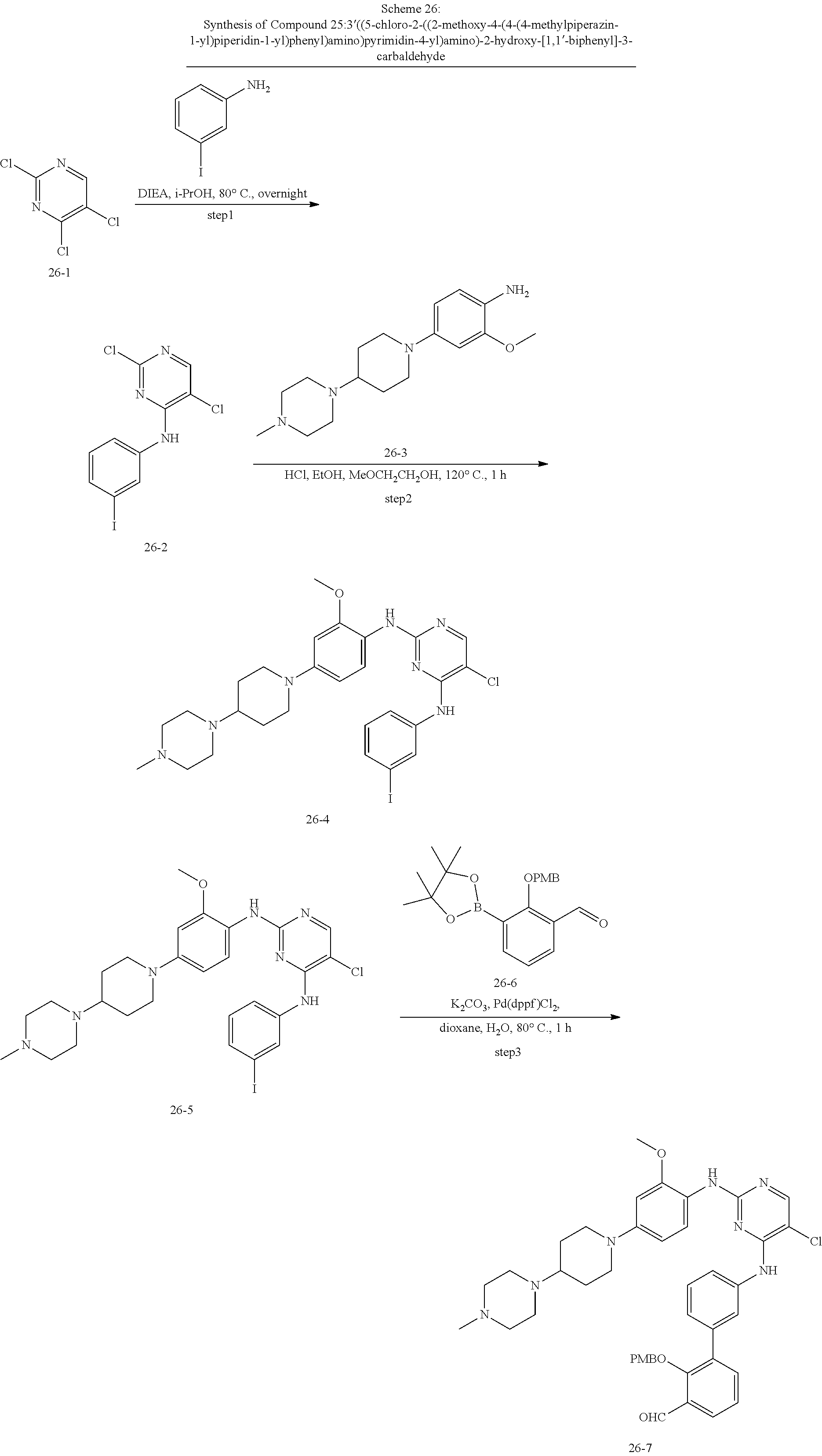

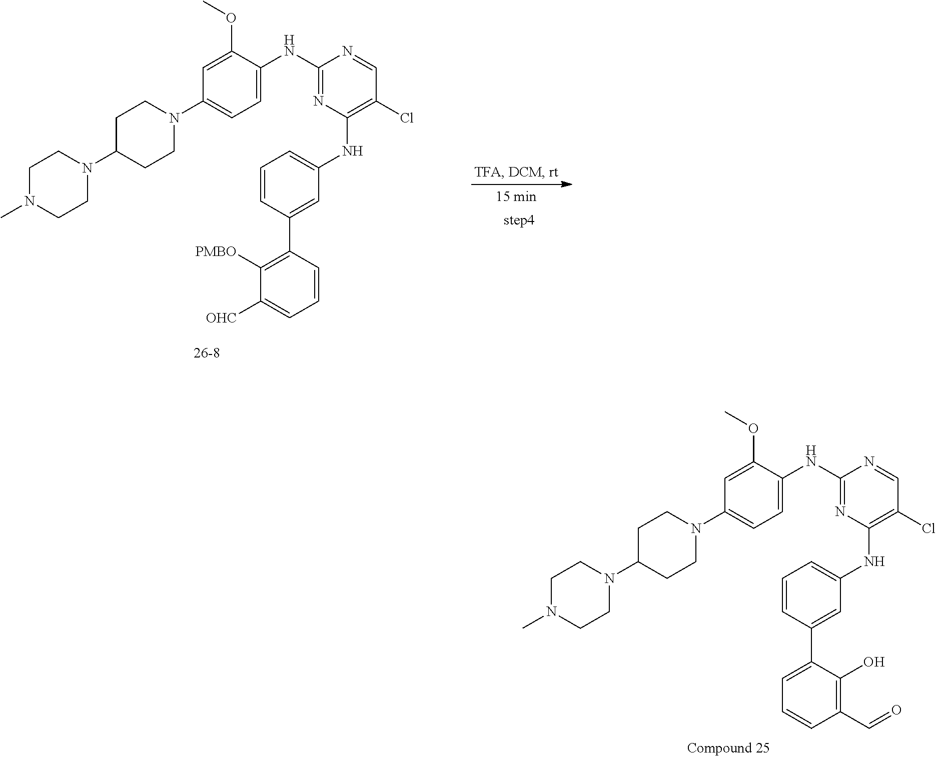

### Step 1: Synthesis of 2,5-dichloro-N-(3-iodophenyl)pyrimidin-4-amine

2,4,5-trichloropyrimidine (1 g, 5.452 mmol, 1 equiv) was added to a 100 mL reaction vessel, then a stir bar, and then added i-PrOH (15 mL) and DIEA (1409.30 mg, 10.904 mmol, 2 equiv) to a 100 mL reaction vessel and stirred until homogeneous, and finally added benzenamine, 3-iodo- (1194.12 mg, 5.452 mmol, 1 equiv) and stirred well. The reaction was stirred overnight at 80° C. Nitrogen protection throughout the reaction process. After cooling down to rt, the reaction was quenched with water 20 ml. The aqueous layer was extracted with EA (30 ml). The residue was purified by silica gel column chromatography, eluted with Mobile Phase A: petroleum ether, Mobile Phase B: ethyl acetate; Flow rate: 60 mL/min; Gradient: 15% B to 25% B, 17% B; Wave Length: 254/220 nm; to afford 2,5-dichloro-N-(3-iodophenyl)pyrimidin-4-amine (1.6988 g, 85.14%). the product was concentrated to dryness to give yellow solid. MS(ESI), calculated for C₁₀H₆Cl₂IN₃: 364.89 m/z found 365.90[M+H]⁺.

### Step 2: Synthesis of 5-chloro-N4-(3-iodophenyl)-N2-(2-methoxy-4-(4-(4-methylpiperazin-1-yl)piperidin-1-yl)phenyl)pyrimidine-2,4-diamine

2,5-dichloro-N-(3-iodophenyl)pyrimidin-4-amine (500 mg, 1.366 mmol, 1 equiv) and 2-butanol (4 mL) were added to the 20 ml microwave tube, a stir bar, then added p-Toluenesulfonic acid (47.05 mg, 0.273 mmol, 0.2 equiv) and 2-methoxy-4-[4-(4-methylpiperazin-1-yl)piperidin-1-yl]aniline (415.92 mg, 1.366 mmol, 1 equiv) to a 20 ml microwave tube and stirred until homogeneous. The reaction was stirred overnight at 120° C. Nitrogen protection throughout the reaction process. Desired product was detected by LCMS. The reaction was quenched by the addition of water 5 ml. The resulting mixture was extracted with EA50 ml. The combined organic layers, dried over anhydrous sodium sulfete.

After filtration, the filtrate was concentrated under reduced pressure. The residue was purified by reverse flash chromatography with the following conditions: column, C18 silica gel; mobile phase, water and MEOH, 60% to 70% gradient in 10 min; detector, UV 254 nm. to afford 5-chloro-N4-(3-iodophenyl)-N2-{2-methoxy-4-[4-(4-methylpiperazin-1-yl)piperidin-1-yl]phenyl}pyrimidine-2,4-diamine (300 mg, 34.64%) as a black soild.MS(ESI),Calculated for C₂₇H₃₃Cl₁N₇O_{:633.15}m/z,found 634.45[M+H]⁺.

### Step 3: Synthesis of 3′-((5-chloro-2-((2-methoxy-4-(4-(4-methylpiperazin-1-yl)piperidin-1-yl)phenyl)amino)pyrimidin-4-yl)amino)-2-((4-methoxybenzyl)oxy)-[1,1′-biphenyl]-3-carbaldehyde

5-chloro-N4-(3-iodophenyl)-N2-{2-methoxy-4-[4-(4-methylpiperazin-1-yl)piperidin-1-yl]phenyl}pyrimidine-2,4-diamine (100 mg, 0.158 mmol, 1 equiv)) and dioxane (4 mL) were added to the 40 ml reaction vessel, a stir bar, then added Pd(dppf)Cl₂ (11.54 mg, 0.016 mmol, 0.1 equiv);K₂CO₃ (43.60 mg, 0.316 mmol, 2 equiv) and 2-[(4-methoxyphenyl)methoxy]-3-(4,4,5,5-tetramethyl-1,3,2-dioxaborolan-2-yl)benzaldehyde (116.17 mg, 0.316 mmol, 2 equiv) to a 40 ml reaction vessel and stirred until homogeneous. The reaction was stirred overnight at 80° C. Nitrogen protection throughout the reaction process. desired product was detected by LCMS.The reaction was quenched with water,The resulting mixture was extracted with ethyl acetate 100 ml.The residue was purified by Prep-TLC (DCM/MEOH=10:1) to afford 3′-{[5-chloro-2-({2-methoxy-4-[4-(4-methylpiperazin-1-yl)piperidin-1-yl]phenyl}amino)pyrimidin-4-yl]amino}-2-[(4-methoxyphenyl)methoxy]-[1,1′-biphenyl]-3-carbaldehyde (83 mg, 70.31%) as a black soild. MS(ESI),Calculated for C₄₂H₄₆ClN₇O4:747.33m/z,found 748.45[M+H]⁺.

### Step 4: Synthesis of 3′-((5-chloro-2-((2-methoxy-4-(4-(4-methylpiperazin-1-yl)piperidin-1-yl)phenyl)amino)pyrimidin-4-yl)amino)-2-hydroxy-[1,1′-biphenyl]-3-carbaldehyde

3′-{[5-chloro-2-({2-methoxy-4-[4-(4-methylpiperazin-1-yl)piperidin-1-yl]phenyl}amino)pyrimidin-4-yl]amino}-2-[(4-methoxyphenyl)methoxy]-[1,1′-biphenyl]-3-carbaldehyde (80 mg, 0.107 mmol, 1 equiv) was added to the 40 ml reaction vessel, a stir bar, then added HCl in dioxane (2 mL) to a 40 ml reaction vessel and stirred until homogeneous.

The reaction was stirred 2h at rt. The resulting mixture was concentrated under reduced pressure. The residue was purified by preparative HPLC using a Column: Xselect CSH C18 OBD Column 30*150 mm 5 μm, n; Mobile Phase A: Water(10 mmol/L NH₄HCO₃), Mobile Phase B: ACN; Flow rate: 60 mL/min; Gradient: 40% B to 60% B in 10 min, 60% B; Wave Length: 254/220 nm; RTT(min): 8.9; Number Of Runs: 0 to afford 3′-{[5-chloro-2-({2-methoxy-4-[4-(4-methylpiperazin-1-yl)piperidin-1-yl]phenyl}amino)pyrimidin-4-yl]amino}-2-hydroxy-[1,1′-biphenyl]-3-carbaldehyde (15 mg, 22.28%) as a yellow solid. MS(ESI), calculated for C₃₄H₃₈ClN₇O3:627.27m/z, found 628.25[M+H]⁺. ¹H NMR (300 MHz, DMSO-d6) δ 11.39 (s, 1H), 10.11 (s, 1H), 8.81 (s, 1H), 8.07 (s, 1H), 7.92-7.86 (m, 1H), 7.82 (dd, J=7.7, 1.8 Hz, 1H), 7.76-7.65 (m, 2H), 7.70-7.60 (m, 1H), 7.52 (d, J=8.7 Hz, 1H), 7.42-7.30 (m, 2H), 7.25-7.12 (m, 1H), 6.54 (d, J=2.5 Hz, 1H), 6.07 (d, J=8.0 Hz, 1H), 3.76 (s, 3H), 3.54 (d, J=12.1 Hz, 2H), 2.61-2.50 (m, 5H), 2.36-2.30 (m, 5H), 2.34-2.20 (m, 1H), 2.16 (s, 3H), 1.88-1.77 (m, 2H), 1.57-1.38 (m, 2H).

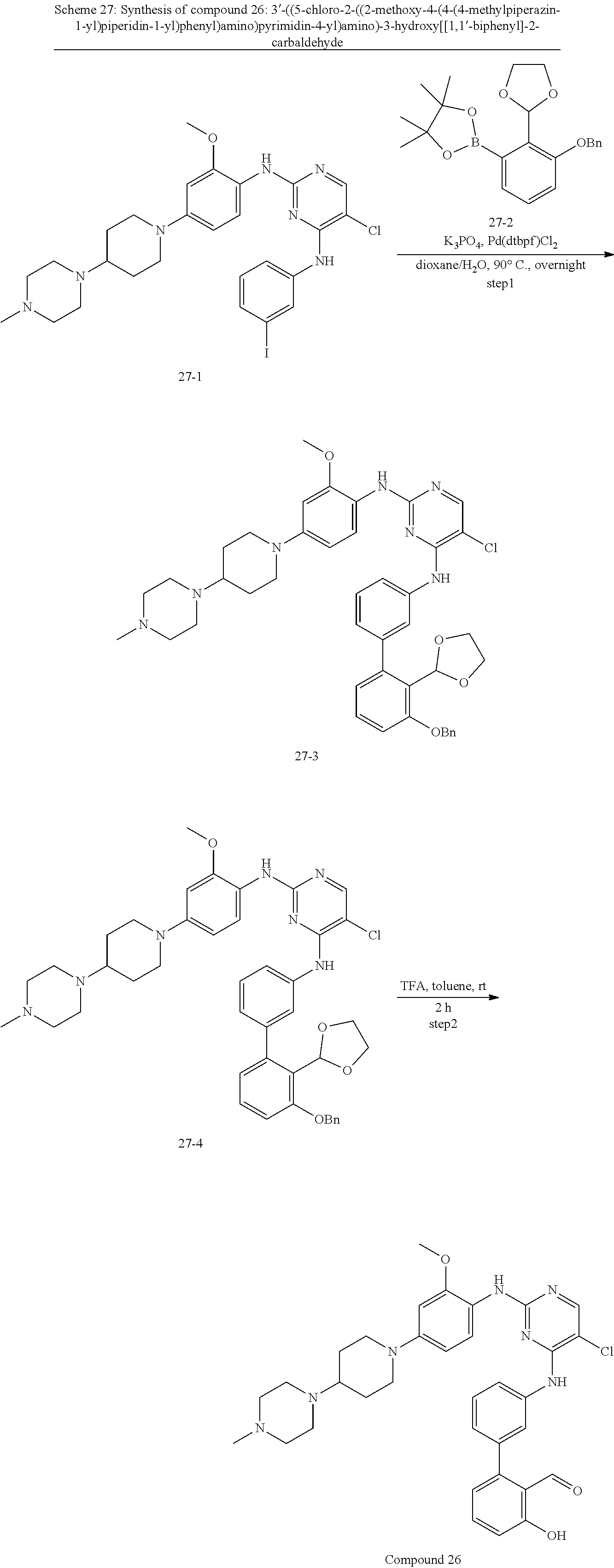 [SMILES: COc1cc(N2CCC(N3CCN(C)CC3)CC2)ccc1Nc1ncc(Cl)c(Nc2cccc(I)c2)n1 ; CC1(C)OB(c2cccc(OCc3ccccc3)c2C2OCCO2)OC1(C)C ; COc1cc(N2CCC(N3CCN(C)CC3)CC2)ccc1Nc1ncc(Cl)c(Nc2cccc(-c3cccc(OCc4ccccc4)c3C3OCCO3)c2)n1 ; COc1cc(N2CCC(N3CCN(C)CC3)CC2)ccc1Nc1ncc(Cl)c(Nc2cccc(-c3cccc(OCc4ccccc4)c3C3OCCO3)c2)n1 ; COc1cc(N2CCC(N3CCN(C)CC3)CC2)ccc1Nc1ncc(Cl)c(Nc2cccc(-c3cccc(O)c3C=O)c2)n1]

### Step 1: Synthesis of N4-(3′-(benzyloxy)-2′-(1,3-dioxolan-2-yl)-[1,1′-biphenyl]-3-yl)-S-chloro-N2-(2-methoxy-4-(4-(4-methylpiperazin-1-yl)piperidin-1-yl)phenyl)pyrimidine-2,4-diamine

5-chloro-N4-(3-iodophenyl)-N2-{2-methoxy-4-[4-(4-methylpiperazin-1-yl)piperidin-1-yl]phenyl}pyrimidine-2,4-diamine (100 mg, 0.158 mmol, 1 equiv) and dioxane (2 mL); H₂O (0.4 mL) were added to the 40 ml reaction vessel, a stir bar, then added Pd(dTBPF)Cl₂(10.28 mg, 0.016 mmol, 0.1 equiv); K3PO₄ (100.45 mg, 0.474 mmol, 3 equiv) and 2-[3-(benzyloxy)-2-(1,3-dioxolan-2-yl)phenyl]-4,4,5,5-tetramethyl-1,3,2-dioxaborolane (120.59 mg, 0.316 mmol, 2 equiv) to a 40 ml reaction vessel and stirred until homogeneous. The reaction was stirred overnight at 80° C. Nitrogen protection throughout the reaction process. The desired product was detected by LCMS. The reaction was quenched with water 3 mL. The resulting mixture was extracted with ethyl acetate 30 mL. The residue was purified by Prep-TLC (DCM/MEOH=10:1) to afford N4-[3′-(benzyloxy)-2′-(1,3-dioxolan-2-yl)-[1,1′-biphenyl]-3-yl]-S-chloro-N2-{2-methoxy-4-[4-(4-methylpiperazin-1-yl)piperidin-1-yl]phenyl}pyrimidine-2,4-diamine (80 mg, 66.53%) as a brown solid. MS(ESI), calculated for C₄₃H₄₈ClN₇O_{4:761.35}m/z, found 762.40[M+H]⁺.

### Step 2: Synthesis of 3′-((5-chloro-2-((2-methoxy-4-(4-(4-methylpiperazin-1-yl)piperidin-1-yl)phenyl)amino)pyrimidin-4-yl)amino)-3-hydroxy-[1,1′-biphenyl]-2-carbaldehyde

N4-[3′-(benzyloxy)-2′-(1,3-dioxolan-2-yl)-[1,1′-biphenyl]-3-yl]-S-chloro-N2-{2-methoxy-4-[4-(4-methylpiperazin-1-yl)piperidin-1-yl]phenyl}pyrimidine-2,4-diamine (100 mg, 0.131 mmol, 1 equiv) was added to the 100 ml reaction vessel, a stir bar, then added TFA (2 mL, 26.926 mmol) toluene (2 mL, 18.797 mmol) to a 100 mL reaction vessel and stirred until homogeneous. The reaction was stirred 2h at r.t, desired product was detected by LCMS. The reaction was quenched with water. The resulting mixture was extracted with ethyl acetate 30 ml. The was purified by preparative HPLC using a Column: XBridge Prep C18 OBD Column, 30*100 mm, 5 μm; Mobile Phase A: Water(10 mmol/L NH₄HCO₃), Mobile Phase B: ACN; Flow rate: 60 mL/min; Gradient: 45% B to 69% B in 9 min, 69% B; Wave Length: 254/220 nm; RT1(min): 7.5; to afford 3′-{[5-chloro-2-({2-methoxy-4-[4-(4-methylpiperazin-1-yl)piperidin-1-yl]phenyl}amino)pyrimidin-4-yl]amino}-3-hydroxy-[1,1′-biphenyl]-2-carbaldehyde (30 mg, 35.97%) as a yellow solid. MS(ESI), calculated for C₃₄H₃₈ClN₇O₃: 627.27m/z, found 628.25 [M+H]⁺¹H NMR (300 MHz, DMSO-d6) δ 11.71 (s, 1H), 9.90 (s, 1H), 8.84 (s, 1H), 8.08 (s, 1H), 7.89-7.77 (m, 2H), 7.71 (t, J=1.9 Hz, 1H), 7.68-7.58 (m, 1H), 7.46 (d, J=8.7 Hz, 1H), 7.38 (t, J=7.9 Hz, 1H), 7.20-7.10 (m, 1H), 7.08-6.93 (m, 2H), 6.56 (d, J=2.5 Hz, 1H), 6.25-6.16 (m, 1H), 3.76 (s, 3H), 3.61 (d, J=11.9 Hz, 2H), 2.66-2.52 (m, 6H), 2.29 (m, 5H), 2.16 (s, 3H), 1.90-1.79 (m, 2H), 1.59-1.40 (m, 2H).

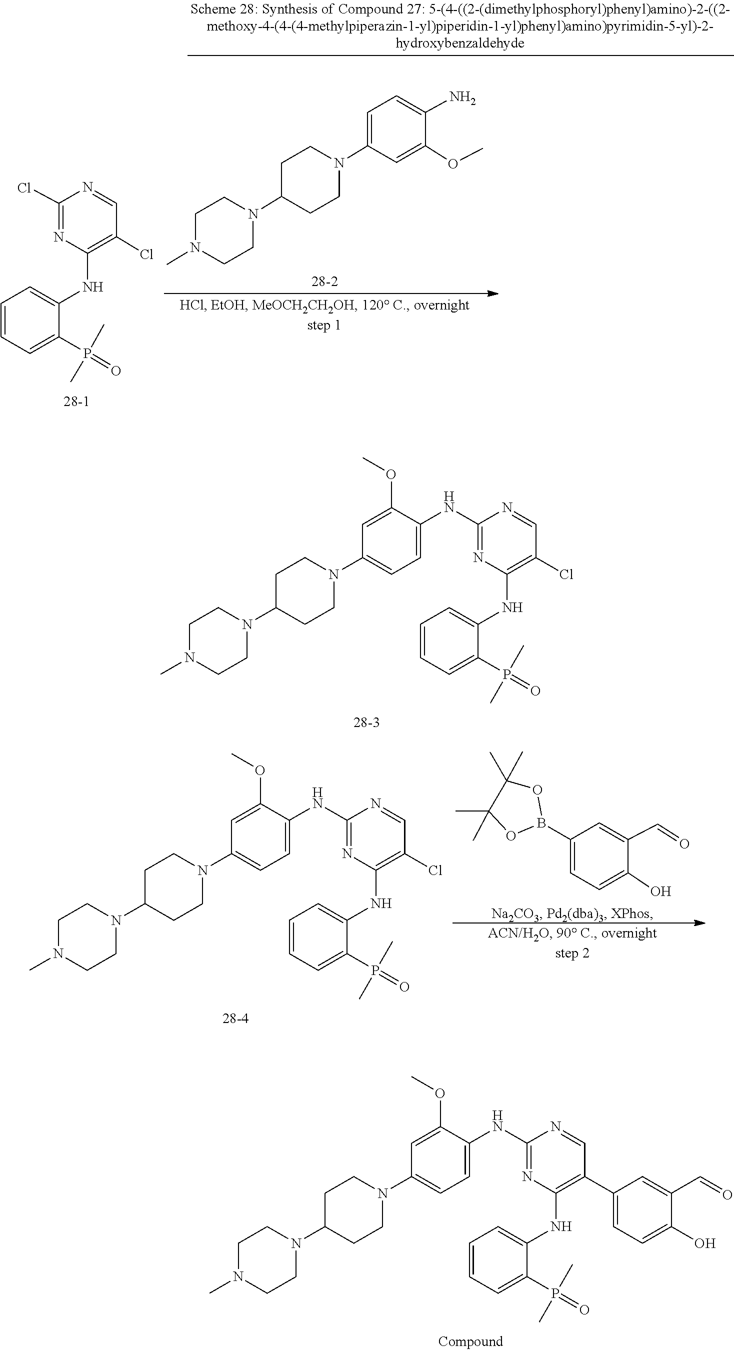

### Step 1: Synthesis of (2-((5-chloro-2-((2-methoxy-4-(4-(4-methylpiperazin-1-yl)piperidin-1-yl)phenyl)amino)pyrimidin-4-yl)amino)phenyl)dimethylphosphine oxide

2,5-dichloro-N-[2-(dimethylphosphoryl)phenyl]pyrimidin-4-amine (200 mg, 0.633 mmol, 1 equiv), a stir bar, 2-ethoxyethanol (5 mL), HCl (28.83 mg, 0.790 mmol, 2.5 equiv) and hydrochloric acid methanol solution (57.67 mg, 1.583 mmol, 2.5 equiv) were added to a 100 mL reaction vessel and stirred until homogeneous. Finally added 2-methoxy-4-[4-(4-methylpiperazin-1-yl)piperidin-1-yl]aniline (192.61 mg, 0.633 mmol, 1 equiv) to the reaction vessel and stirred well. The reaction was stirred overnight at 120° C. Nitrogen protection throughout the reaction process. After cooling down to rt, the reaction mixture was concentrated to dryness to give black solid. The black solid was purified by preparative HPLC using a XSelect CSH Fluoro Phenyl, 30*150 mm, 5 μm column (eluent: 1% to 80% (v/v) MEOH and H₂O with 10 mmol/L NH₄HCO₃) to afford brigatinib (320 mg, 86.59%) as a black solid. MS(ESI), calculated for C₂₉H₃₉ClN₇O₂P:583.25m/z, found 584.30[M+H]⁺.

### Step 2: Synthesis of 5-(4-((2-(dimethylphosphoryl)phenyl)amino)-2-((2-methoxy-4-(4-(4-methylpiperazin-1-yl)piperidin-1-yl)phenyl)amino)pyrimidin-S-yl)-2-hydroxybenzaldehyde

Brigatinib (100 mg, 0.171 mmol, 1 equiv) and ACN (4 mL) H₂O (0.8 mL) were added to a 40 mL reaction vessel, Then a stir bar, and then added Na₂CO₃ (36.29 mg, 0.342 mmol, 2 equiv);Pd₂(dba)₃ (15.68 mg, 0.017 mmol, 0.1 equiv) and XPhos (8.16 mg, 0.017 mmol, 0.1 equiv) to a 40 mL reaction vessel and stirred until homogeneous. Finally added 2-hydroxy-S-(4,4,5,5-tetramethyl-1,3,2-dioxaborolan-2-yl)benzaldehyde (84.95 mg, 0.342 mmol, 2 equiv) and stirred well. The reaction was stirred overnight at 120° C. Nitrogen protection throughout the reaction process. After cooling down to rt, the reaction mixture was concentrated to dryness to give yellow solid. The yellow solid was purified by preparative HPLC using a Column: XBridge Shield RP18 OBD Column, 19*250 mm, 10 μm; Mobile Phase A: Water(10 mmol/L NH₄HCO₃+0.1% NH₃·H₂O), Mobile Phase B: MeOH—-HPLC; Flow rate: 25 mL/min; Gradient: 55% B to 75% B in 7 min, 75% B; Wave Length: 254 nm; RTT(min): 6.5; Number Of Runs: 0 to afford 2-hydroxy-S-[2-({2-methoxy-4-[4-(4-methylpiperazin-1-yl)piperidin-1-yl]phenyl}amino)-4-(phenylamino)pyrimidin-S-yl]benzaldehyde (40 mg, 34.11%) as a yellow solid. MS(ESI), calculated for C₃₆H₄₄N₇O₄P:669.76 m/z, found 670.35[M+H]⁺. ¹H NMR (300 MHz, DMSO-d6) δ 10.43 (s, 1H), 10.26 (s, 1H), 8.54-8.44 (m, 1H), 7.89 (s, 1H), 7.83 (s, 1H), 7.67 (d, J=2.4 Hz, 1H), 7.63-7.27 (m, 4H), 7.10-6.97 (m, 2H), 6.64 (d, J=2.5 Hz, 1H), 6.46 (dd, J=8.8, 2.5 Hz, 1H), 3.80 (s, 3H), 3.71 (d, J=12.0 Hz, 2H), 2.66 (m, 7H), 2.31 (m, 5H), 2.15 (s, 3H), 1.86 (d, J=12.3 Hz, 2H), 1.61-1.48 (m, 8H).

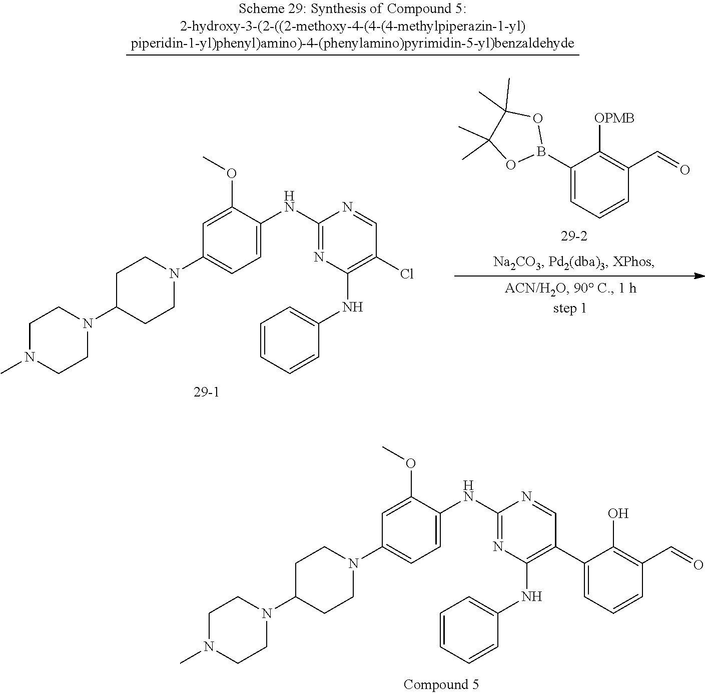

### Step 1: Synthesis of 2-hydroxy-3-(2-((2-methoxy-4-(4-(4-methylpiperazin-1-yl)piperidin-1-yl)phenyl)amino)-4-(phenylamino)pyrimidin-S-yl)benzaldehyde

ACN (3 mL, 0.394 mmol);H₂O (0.6 mL) and 5-chloro-N2-{2-methoxy-4-[4-(4-methylpiperazin-1-yl)piperidin-1-yl]phenyl}-N4-phenylpyrimidine-2,4-diamine (100 mg, 0.197 mmol, 1 equiv) were added to a 40 mL reaction vessel with a stir bar. Then added Na₂CO₃ (41.72 mg, 0.394 mmol, 2 equiv); Pd₂(dba)₃ (18.02 mg, 0.020 mmol, 0.1 equiv) and XPhos (9.38 mg, 0.020 mmol, 0.1 equiv) to a 40 mL reaction vessel and stirred until homogeneous.

Finally added 2-[(4-methoxyphenyl)methoxy]-3-(4,4,5,5-tetramethyl-1,3,2-dioxaborolan-2-yl)benzaldehyde (108.72 mg, 0.295 mmol, 1.5 equiv) and stir well. The reaction was stirred overnight at 90° C. Nitrogen protection throughout the reaction process. After cooling down to rt, the reaction mixture was concentrated to dryness to give yellow solid. The yellow solid was purified by preparative HPLC using a Gradient: isocratic to afford 2-hydroxy-3-[2-({2-methoxy-4-[4-(4-methylpiperazin-1-yl)piperidin-1-yl]phenyl}amino)-4-(phenylamino)pyrimidin-S-yl]benzaldehyde (5 mg, 4.27%) as a yellow solid. MS(ESI), calculated for C₃₄H₃₉N₇O₃:593.31m/z, found 594.30[M+H]⁺. ¹H NMR (300 MHz, DMSO-d6) δ 10.22 (s, 1H), 9.42-9.02 (m, 1H), 7.88-7.82 (m, 2H), 7.72 (dd, J=14.8, 8.1 Hz, 1H), 7.65-7.56 (m, 1H), 7.56-7.39 (m, 4H), 7.22 (t, J=7.8 Hz, 2H), 7.02-6.85 (m, 2H), 6.62 (d, J=2.5 Hz, 1H), 6.41 (dd, J=8.9, 2.5 Hz, 1H), 3.81 (s, 3H), 3.74-3.59 (m, 4H), 2.70-2.47 (m, 4H), 2.44-2.37 (m, 2H), 2.35-2.29 (m, 3H), 2.20 (s, 3H), 1.91-1.81 (m, 2H), 1.62-1.44 (m, 2H).

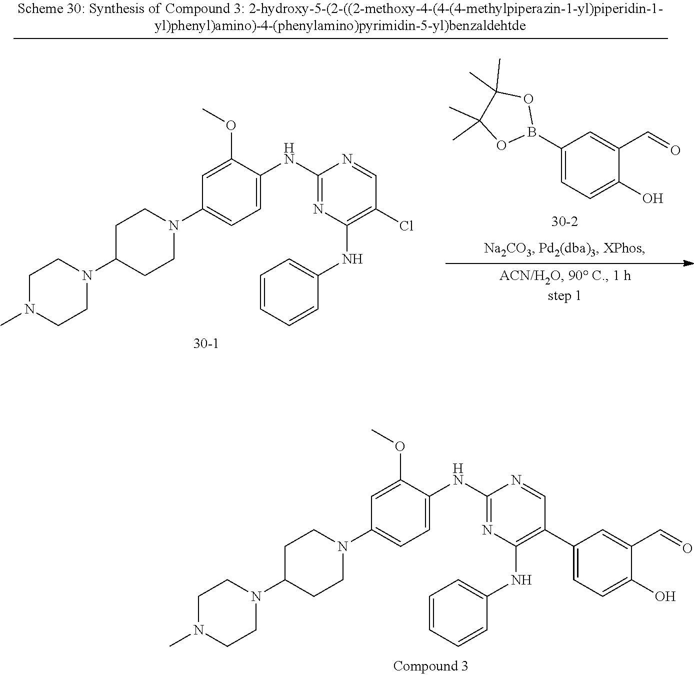 [SMILES: COc1cc(N2CCC(N3CCN(C)CC3)CC2)ccc1Nc1ncc(Cl)c(Nc2ccccc2)n1 ; CC1(C)OB(c2ccc(O)c(C=O)c2)OC1(C)C ; COc1cc(N2CCC(N3CCN(C)CC3)CC2)ccc1Nc1ncc(-c2ccc(O)c(C=O)c2)c(Nc2ccccc2)n1]

### Step 1: Synthesis of 2-hydroxy-S-(2-((2-methoxy-4-(4-(4-methylpiperazin-1-yl)piperidin-1-yl)phenyl)amino)-4-(phenylamino)pyrimidin-S-yl)benzaldehyde

ACN (8 mL, 152.194 mmol) and 5-chloro-N₂-{2-methoxy-4-[4-(4-methylpiperazin-1-yl)piperidin-1-yl]phenyl}-N₄-phenylpyrimidine-2,4-diamine (100 mg, 0.197 mmol, 1 equiv) were added to a 40 mL reaction vessel, Then a stir bar, and then added Na₂CO₃ (41.72 mg, 0.394 mmol, 2 equiv);Pd₂(dba)₃ (18.02 mg, 0.020 mmol, 0.1 equiv) and XPhos (9.38 mg, 0.020 mmol, 0.1 equiv) to a 40 mL reaction vessel and stirred until homogeneous, Finally added 2-hydroxy-S-(4,4,5,5-tetramethyl-1,3,2-dioxaborolan-2-yl)benzaldehyde (73.24 mg, 0.295 mmol, 1.5 equiv) and stirred well. The reaction was stirred overnight at 90° C. Nitrogen protection throughout the reaction process. After cooling down to rt, the reaction mixture was concentrated to dryness to give yellow solid. The yellow solid was purified by preparative HPLC using a Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 20% B in 8 min, 20% B; Wave Length: 254/220 nm; RT1(min): 8; Number Of Runs: 0 to afford 2-hydroxy-S-[2-({2-methoxy-4-[4-(4-methylpiperazin-1-yl)piperidin-1-yl]phenyl}amino)-4-(phenylamino)pyrimidin-S-yl]benzaldehyde (40 mg, 34.11%) as a yellow solid. MS(ESI), calculated for C₃₄H₃₉N₇O₃:593.73m/z, found 594.35[M+H]⁺. ¹H NMR (300 MHz, DMSO-d6) δ 10.31 (s, 1H), 8.21 (s, 1H), 8.12 (s, 1H), 7.86 (s, 1H), 7.72-7.62 (m, 3H), 7.62-7.51 (m, 3H), 7.23 (t, J=7.9 Hz, 2H), 7.11 (d, J=8.5 Hz, 1H), 7.06-6.94 (m, 1H), 6.62 (d, J=2.4 Hz, 1H), 6.40 (dd, J=8.8, 2.5 Hz, 1H), 3.80 (s, 3H), 3.68 (d, J=12.0 Hz, 2H), 2.70-2.52 (m, 5H), 2.45-2.26 (m, 6H), 2.20 (s, 3H), 1.91-1.81 (m, 2H), 1.61-1.44 (m, 2H).

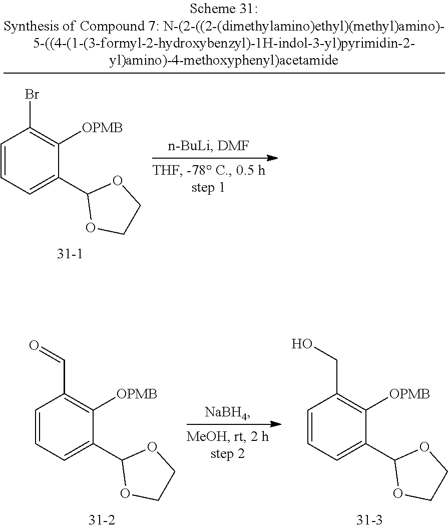

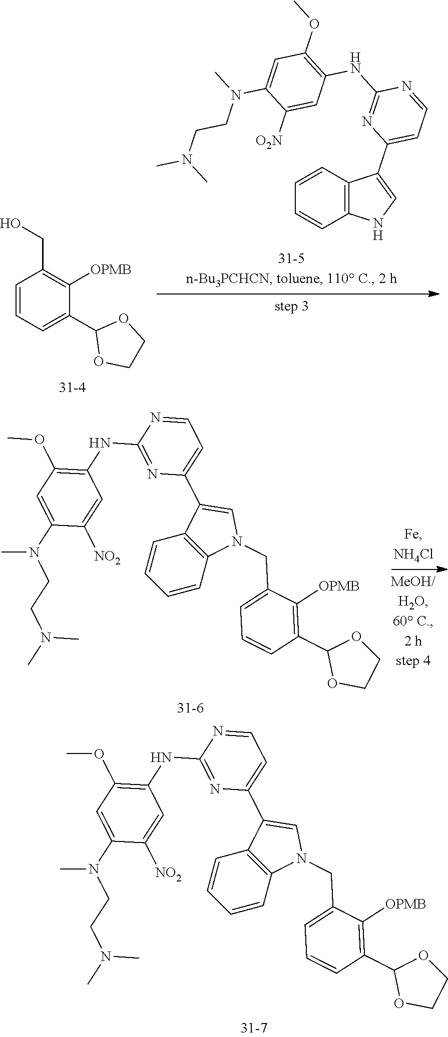

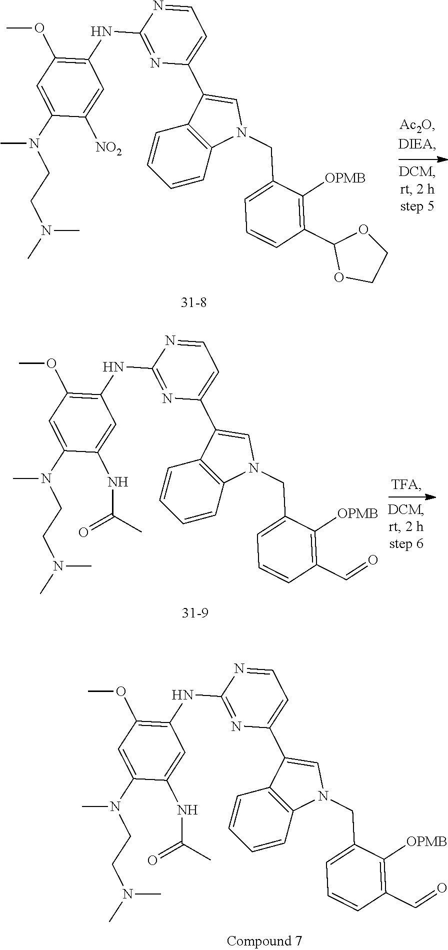

### Step 1: Synthesis of 3-(1,3-dioxolan-2-yl)-2-((4-methoxybenzyl)oxy)benzaldehyde

2-{3-bromo-2-[(4-methoxyphenyl)methoxy]phenyl}-1,3-dioxolane (1 g, 2.738 mmol, 1 equiv), a stir bar, THF (10 mL) was added to a 100 mL reaction vessel under nitrogen and stirred at −78° C. (−30 min). Then slow drops plus n-BuLi (2.5M solution in Hexanes, SpcSeal) (1.3 ml) (˜10 min). Then under nitrogen and stirred at −78° C. for 0.5 h. Then slow drops plus DMF (0.40 g, 5.476 mmol, 2 equiv) at −78° C. Then under nitrogen and stirred at −78° C. for 0.5 h. The resulting mixture turned into yellow. The reaction was quenched with water (50 mL). And extracted with ethyl acetate (3×50 mL). The combined organic layer was washed with sat. NaCl (50 ml), dried over anhydrous Na₂SO₄, filtered, and concentrated to afford a yellow solid. It was purified by column chromatography on silica gel with (0-26%, PE/EA) to afford 3-(1,3-dioxolan-2-yl)-2-[(4-methoxyphenyl)methoxy]benzaldehyde (680 mg, 79.01%) as a yellow oil. MS (ESI): mass calculated for C18H₁₈O₅ 314.12 m/z, found 337.00 [M+Na]⁺.

### Step 2: Synthesis of (3-(1,3-dioxolan-2-yl)-2-((4-methoxybenzyl)oxy)phenyl)methanol

3-(1,3-dioxolan-2-yl)-2-[(4-methoxyphenyl)methoxy]benzaldehyde (680 mg, 2.163 mmol, 1 equiv), a stir bar, MeOH (8 mL) was added to a 40 mL reaction vessel and stirred until homogeneous (˜10 min). Then NaBH4 (163.67 mg, 4.326 mmol, 2 equiv) was added and the mixture was evacuated and backfilled with nitrogen at 0° C. The resulting mixture was maintained under nitrogen and stirred at rt for 2 h. The resulting mixture turned into white. the reaction was quenched with water (30 mL). And extracted with ethyl acetate (3×30 mL). The combined organic layer was washed with sat. NaCl (100 ml), dried over anhydrous Na₂SO₄, filtered and concentrated to afford a brown solid. It was purified by column chromatography on silica gel with (0-45%, PE/EA) to afford [3-(1,3-dioxolan-2-yl)-2-[(4-methoxyphenyl)methoxy]phenyl]methanol (600 mg, 87.67%) as a white solid. MS (ESI): mass calculated for C18H₂OO₅ 316.13 m/z, found 317.05 [M+H]⁺.

### Step 3: Synthesis of N1-(4-(1-(3-(1,3-dioxolan-2-yl)-2-((4-methoxybenzyl)oxy)benzyl)-1H-indol-3-yl)pyrimidin-2-yl)-N₄-(2-(dimethylamino)ethyl)-2-methoxy-N₄-methyl-S-nitrobenzene-1,4-diamine

[3-(1,3-dioxolan-2-yl)-2-[(4-methoxyphenyl)methoxy]phenyl]methanol (200 mg, 0.632 mmol, 1 equiv), a stir bar, toluene (5 mL) was added to a 40 mL reaction vessel and stirred until homogeneous (˜10 min). Then, N1-[2-(dimethylamino)ethyl]-N4-[4-(1H-indol-3-yl)pyrimidin-2-yl]-S-methoxy-N1-methyl-2-nitrobenzene-1,4-diamine (437.67 mg, 0.948 mmol, 1.5 equiv), 2-(tributyl-lambda-S-phosphanylidene)acetonitrile (305.18 mg, 1.264 mmol, 2 equiv) was added and the mixture was evacuated and backfilled with nitrogen at rt. The resulting mixture was maintained under nitrogen and stirred at 120° C. for 2 h. The resulting mixture turned into black. the reaction was quenched with water (50 mL). And extracted with ethyl acetate (3×50 mL). The combined organic layer was washed with sat. NaCl (50 ml), dried over anhydrous Na₂SO₄, filtered and concentrated to afford a black solid. It was purified by column chromatography on silica gel with (0-76%, PE/EA) to afford N1-[2-(dimethylamino)ethyl]-N4-[4-(1-{[3-(1,3-dioxolan-2-yl)-2-[(4-methoxyphenyl)methoxy]phenyl]methyl}indol-3-yl)pyrimidin-2-yl]-S-methoxy-N1-methyl-2-nitrobenzene-1,4-diamine (300 mg, 62.45%) as a brown solid solid. MS (ESI): mass calculated for C₄₂H₄₅N₇O₇ 759.34 m/z, found 760.25 [M+H]⁺.

### Step 4: Synthesis of N4-(4-(1-(3-(1,3-dioxolan-2-yl)-2-((4-methoxybenzyl)oxy)benzyl)-1H-indol-3-yl)pyrimidin-2-yl)-N1-(2-(dimethylamino)ethyl)-S-methoxy-N1-methylbenzene-1,2,4-triamine

N1-[2-(dimethylamino)ethyl]-N4-[4-(1-{[3-(1,3-dioxolan-2-yl)-2-[(4-methoxyphenyl)methoxy]phenyl] methyl}indol-3-yl)pyrimidin-2-yl]-S-methoxy-N1-methyl-2-nitrobenzene-1,4-diamine (270 mg, 0.355 mmol, 1 equiv), a stir bar, MeOH (6 mL),H₂O (2 mL) was added to a 40 mL reaction vessel and stirred until homogeneous (˜10 min). Then Fe (158.75 mg, 2.840 mmol, 8 equiv), NH₄C₁ (152.05 mg, 2.840 mmol, 8 equiv) was added and the mixture was evacuated and backfilled with nitrogen at rt The resulting mixture was maintained under nitrogen and stirred at 60° C. for 2 h. The resulting mixture turned into black and was filtered and concentrated to N1-[2-(dimethylamino)ethyl]-N4-[4-(1-{[3-(1,3-dioxolan-2-yl)-2-[(4-methoxyphenyl) methoxy]phenyl]methyl}indol-3-yl)pyrimidin-2-yl]-S-methoxy-N1-methylbenzene-1,2,4-triamine (250 mg, 96.40%) as a brown solid. MS (ESI): mass calculated for C₄₂H₄₇N₇O₅ 729.36m/z, found 730.30 [M+H]⁺.

### Step 5: Synthesis of N-(2-((2-(dimethylamino)ethyl)(methyl)amino)-S-((4-(1-(3-formyl-2-((4-methoxy benzyl)oxy)benzyl)-1H-indol-3-yl)pyrimidin-2-yl)amino)-4-methoxyphenyl)acetamide

N1-[2-(dimethylamino)ethyl]-N4-[4-(1-{[3-(1,3-dioxolan-2-yl)-2-[(4-methoxyphenyl)methoxy]phenyl] methyl} indol-3-yl)pyrimidin-2-yl]-S-methoxy-N1-methylbenzene-1,2,4-triamine (250 mg, 0.343 mmol, 1 equiv), a stir bar, DCM (4 mL) was added to a 40 mL reaction vessel and stirred until homogeneous (˜10 min). Then, DIEA (132.81 mg, 1.029 mmol, 3 equiv), Ac₂O (69.94 mg, 0.686 mmol, 2 equiv) was added, and the mixture was evacuated and backfilled with nitrogen at rt. The resulting mixture was maintained under nitrogen and stirred at rt for 2 h. The resulting mixture turned into black. The reaction was quenched with water (50 mL) and extracted with ethyl acetate (3×50 mL). The combined organic layer was washed with sat. NaCl (50 ml), dried over anhydrous Na₂SO₄, filtered and concentrated to afford a black solid. It was purified by column chromatography on silica gel with (0-69%, PE/EA) to afford N-(2-{[2-(dimethylamino)ethyl](methyl)amino}-S-({4-[1-({3-formyl-2-[(4-methoxyphenyl)methoxy]phenyl}methyl) indol-3-yl]pyrimidin-2-yl}amino)-4-methoxyphenyl)acetamide (130 mg, 52.14%) as a light yellow oil. MS (ESI): mass calculated for C₄₂H₄₅N₇O₅ 727.35 m/z, found 728.50 [M+H]⁺.

### Step 6: Synthesis of N-(2-((2-(dimethylamino)ethyl)(methyl)amino)-S-((4-(1-(3-formyl-2-hydroxy benzyl)-1H-indol-3-yl)pyrimidin-2-yl)amino)-4-methoxyphenyl)acetamide

N-(5-((4-(1-(3-(1,3-dioxolan-2-yl)-2-((4-methoxybenzyl)oxy)benzyl)-1H-indol-3-yl)pyrimidin-2-yl)amino)-2-((2-(dimethylamino)ethyl)(methyl)amino)-4-methoxyphenyl)acetamide (120 mg, 0.155 mmol), a stir bar, DCM (4 mL) was added to a 40 mL reaction vessel and stirred until homogeneous (˜10 min). Then trifluoroacetic acid (1 mL) was added and the mixture was evacuated and backfilled with nitrogen at rt. The resulting mixture was maintained under nitrogen and stirred at rt for 2 h. The resulting mixture turned into yellow. the reaction was quenched with water (50 mL). And extracted with ethyl acetate (3×50 mL). The combined organic layer was washed with sat. NaCl (50 ml), dried over anhydrous Na₂SO₄, filtered and concentrated to afford a white solid. It was purified by column chromatography on silica gel with (0-4%, DCM/MeOH) and preparative HPLC using Column: XBridge Shield RP18 OBD Column, 30*150 mm, 5 μm; Mobile Phase A: Water(0.05% TFA ), Mobile Phase B: ACN; Flow rate: 60 mL/min; Gradient: 17% B to 38% B in 8 min, 38% B; Wave Length: 254/220 nm to afford N-(2-((2-(dimethylamino)ethyl)(methyl)amino)-S-((4-(1-(3-formyl-2-hydroxybenzyl)-1H-indol-3-yl)pyrimidin-2-yl)amino)-4-methoxyphenyl)acetamide as a yellow solid (12 mg, 12.68%). MS (ESI): mass calculated for C₃₄H₃₇N₇O₄ 607.29 m/z, found 608.30 [M+H]⁺. 1H NMR (300 MHz, DMSO-d6) δ 11.39 (s, 1H), 10.07 (s, 1H), 9.44 (s, 1H), 9.34 (s, 1H), 8.76 (s, 1H), 8.47 (s, 1H), 8.31 (d, J=6.3 Hz, 2H), 7.75 (dd, J=7.7, 1.7 Hz, 1H), 7.59 (d, J=8.2 Hz, 1H), 7.39-7.29 (m, 2H), 7.29-7.20 (m, 1H), 7.16 (t, J=7.5 Hz, 1H), 7.08-6.94 (m, 2H), 5.57 (s, 2H), 3.87 (s, 3H), 3.30 (s, 4H), 2.83 (d, J=4.4 Hz, 6H), 2.65 (s, 3H), 2.10 (s, 3H). 19F NMR (282 MHz, DMSO-d6) δ-73.97, -74.01.

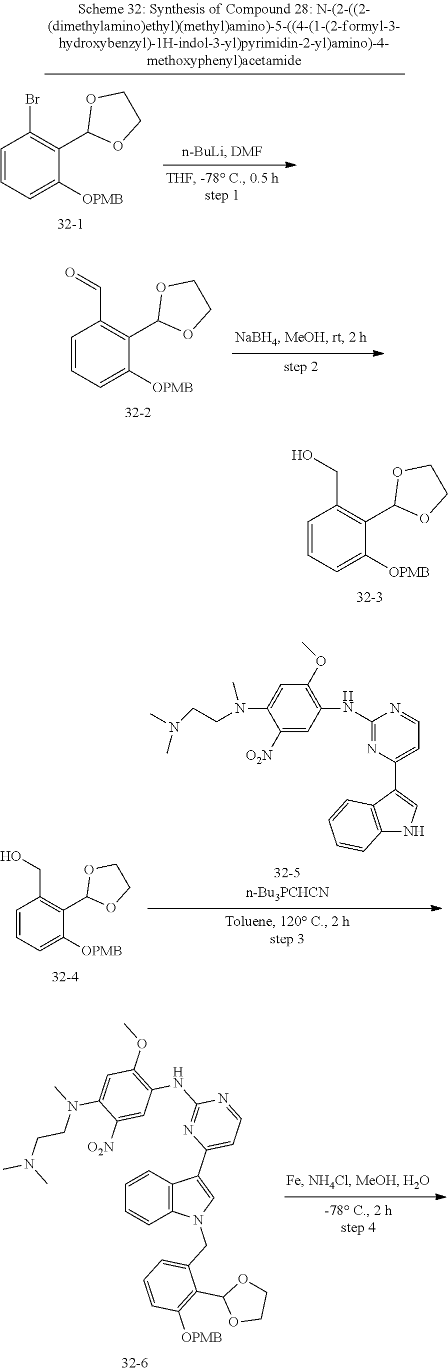

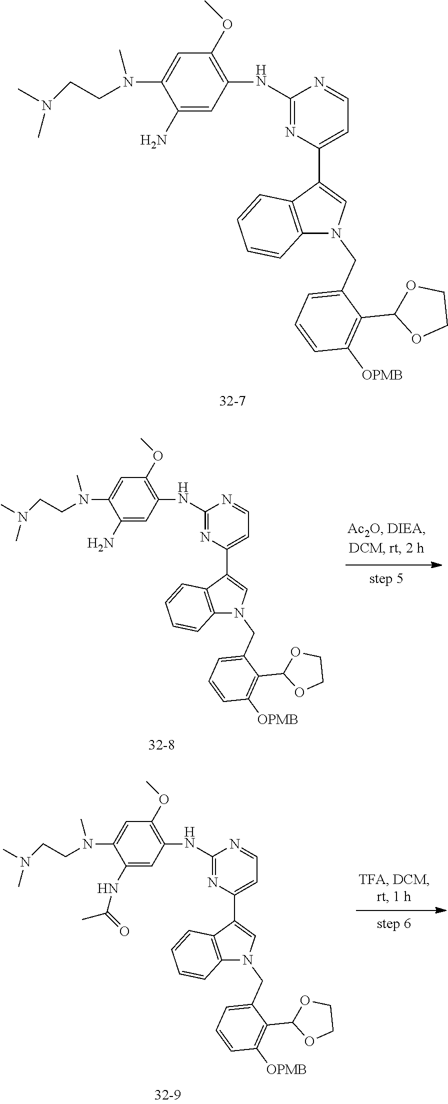

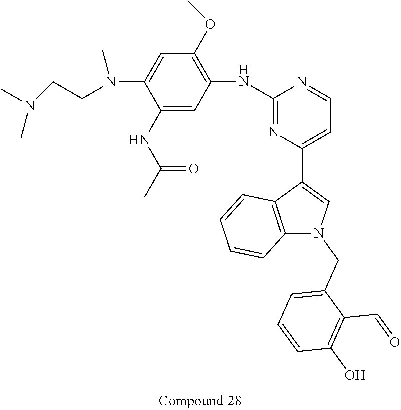 [SMILES: COc1cc(N(C)CCN(C)C)c(NC(C)=O)cc1Nc1nccc(-c2cn(Cc3cccc(O)c3C=O)c3ccccc23)n1]

### Step 1: Synthesis of 2-(1,3-dioxolan-2-yl)-3-((4-methoxybenzyl)oxy)benzaldehyde

2-{2-bromo-6-[(4-methoxyphenyl)methoxy]phenyl}-1,3-dioxolane (1 g, 2.738 mmol, 1 equiv), a stir bar, THF (15 mL) was added to a 100 mL reaction vessel under nitrogen and stirred at −78° C. (˜30 min). Then slow drops plus n-BuLi (2.5M solution in Hexanes, SpcSeal) (1.3 ml) (˜10 min). Then under nitrogen and stirred at −78° C. for 0.5 h. Then slow drops plus DMF (0.40 g, 5.476 mmol, 2 equiv) at −78° C. then under nitrogen and stirred at −78° C. for 0.5 h. The resulting mixture turned into white. The reaction was quenched with water (50 mL) and extracted with ethyl acetate (3×50 mL). The combined organic layer was washed with sat. NaCl (50 ml), dried over anhydrous Na₂SO₄, filtered and concentrated to afford a yellow solid. It was purified by column chromatography on silica gel with (0-23%, PE/EA) to afford 2-(1,3-dioxolan-2-yl)-3-[(4-methoxyphenyl)methoxy]benzaldehyde (660 mg, 76.68%) as a white solid. MS (ESI): mass calculated for C18H₁₈O₅ 314.12 m/z, found 315.20 [M+H]⁺.

### Step 2: Synthesis of (2-(1,3-dioxolan-2-yl)-3-((4-methoxybenzyl)oxy)phenyl)methanol

2-(1,3-dioxolan-2-yl)-3-[(4-methoxyphenyl)methoxy]benzaldehyde (650 mg, 2.068 mmol, 1 equiv), a stir bar, MeOH (8 mL) was added to a 40 mL reaction vessel and stirred until homogeneous (˜10 min). Then NaBH₄ (156.45 mg, 4.136 mmol, 2 equiv) was added and the mixture was evacuated and backfilled with nitrogen at 0° C. The resulting mixture was maintained under nitrogen and stirred at rt for 2 h. The resulting mixture turned into white. the reaction was quenched with water (30 mL). And extracted with ethyl acetate (3×30 mL). The combined organic layer was washed with sat. NaCl (100 mL), dried over anhydrous Na₂SO₄, filtered and concentrated to afford a brown solid. It was purified by column chromatography on silica gel with (0-28%, PE/EA) to afford [2-(1,3-dioxolan-2-yl)-3-[(4-methoxyphenyl)methoxy]phenyl]methanol (600 mg, 91.72%) as a white solid. MS (ESI): mass calculated for C18H₂₀O₅ 316.13 m/z, found 339.00 [M+Na]⁺.

### Step 3: Synthesis of N1-(4-(1-(2-(1,3-dioxolan-2-yl)-3-((4-methoxybenzyl)oxy)benzyl)-1H-indol-3-yl)pyrimidin-2-yl)-N4-(2-(dimethylamino)ethyl)-2-methoxy-N4-methyl-S-nitrobenzene-1,4-diamine

[2-(1,3-dioxolan-2-yl)-3-[(4-methoxyphenyl)methoxy]phenyl]methanol (200 mg, 0.632 mmol, 1 equiv), a stir bar, toluene (4 mL) was added to a 40 mL reaction vessel and stirred until homogeneous (˜10 min). Then N1-[2-(dimethylamino)ethyl]-N4-[4-(1H-indol-3-yl)pyrimidin-2-yl]-S-methoxy-N1-methyl-2-nitrobenzene-1,4-diamine (437.67 mg, 0.948 mmol, 1.5 equiv), 2-(tributyl-lambda5-phosphanylidene)acetonitrile (305.18 mg, 1.264 mmol, 2 equiv) was added and the mixture was evacuated and backfilled with nitrogen at rt. The resulting mixture was maintained under nitrogen and stirred at 120° C. for 2 h. The resulting mixture turned into black. the reaction was quenched with water (50 mL). And extracted with ethyl acetate (3×50 mL). The combined organic layer was washed with sat. NaCl (50 ml), dried over anhydrous Na₂SO₄, filtered, and concentrated to afford a black solid. It was purified by column chromatography on silica gel with (0-79%, PE/EA) to afford N1-[2-(dimethylamino)ethyl]-N4-[4-(1-{[2-(1,3-dioxolan-2-yl)-3-[(4-methoxyphenyl)methoxy]phenyl]methyl}indol-3-yl)pyrimidin-2-yl]-S-methoxy-N1-methyl-2-nitrobenzene-1,4-diamine (220 mg, 45.80%) as a brown solid. MS (ESI): mass calculated for C₄₂H₄₅N₇O₇ 759.34m/z, found 760.30 [M+H]⁺.

### Step 4: Synthesis of N4-(4-(1-(2-(1,3-dioxolan-2-yl)-3-((4-methoxybenzyl)oxy)benzyl)-1H-indol-3-yl)pyrimidin-2-yl)-N1-(2-(dimethylamino)ethyl)-S-methoxy-N1-methylbenzene-1,2,4-triamine

N1-[2-(dimethylamino)ethyl]-N4-[4-(1-{[2-(1,3-dioxolan-2-yl)-3-[(4-methoxyphenyl)methoxy]phenyl]methyl}indol-3-yl)pyrimidin-2-yl]-S-methoxy-N1-methyl-2-nitrobenzene-1,4-diamine (200 mg, 0.263 mmol, 1 equiv), a stir bar, MeOH (3 mL),H₂O (1 mL) was added to a 40 mL reaction vessel and stirred until homogeneous (˜10 min). Then Fe (117.59 mg, 2.104 mmol, 8 equiv), NH₄C₁ (112.63 mg, 2.104 mmol, 8 equiv) was added and the mixture was evacuated and backfilled with nitrogen at rt. The resulting mixture was maintained under nitrogen and stirred at 60° C. for 2 h. The resulting mixture turned into black. filtered and concentrated to N1-[2-(dimethylamino)ethyl]-N4-[4-(1-{[2-(1,3-dioxolan-2-yl)-3-[(4-methoxyphenyl)methoxy]phenyl]methyl}indol-3-yl)pyrimidin-2-yl]-S-methoxy-N1-methylbenzene-1,2,4-triamine (180 mg, 93.70%) as a brown solid.MS (ESI): mass calculated for C₄₂H₄₇N₇O₅ 729.36m/z, found 730.30 [M+H]⁺.

### Step 5: Synthesis of N-(5-((4-(1-(2-(1,3-dioxolan-2-yl)-3-((4-methoxybenzyl)oxy)benzyl)-1H-indol-3-yl)pyrimidin-2-yl)amino)-2-((2-(dimethylamino)ethyl)(methyl)amino)-4-methoxyphenyl)acetamide

N1-[2-(dimethylamino)ethyl]-N4-[4-(1-{[2-(1,3-dioxolan-2-yl)-3-[(4-methoxyphenyl)methoxy]phenyl]methyl}indol-3-yl)pyrimidin-2-yl]-S-methoxy-N1-methylbenzene-1,2,4-triamine (190 mg, 0.260 mmol, 1 equiv), a stir bar, DCM (4 mL) was added to a 40 mL reaction vessel and stirred until homogeneous (˜10 min). Then DIEA (100.94 mg, 0.780 mmol, 3 equiv), Ac₂O (53.15 mg, 0.520 mmol, 2 equiv) was added and the mixture was evacuated and backfilled with nitrogen at rt. The resulting mixture was maintained under nitrogen and stirred at rt for 2 h. The resulting mixture turned into black. The reaction was quenched with water (50 mL). And extracted with ethyl acetate (3×50 mL). The combined organic layer was washed with sat. NaCl (50 ml), dried over anhydrous Na₂SO₄, filtered, and concentrated to afford a black solid. It was purified by column chromatography on silica gel with (0-84%, PE/EA) to afford N-(2-{[2-(dimethylamino)ethyl](methyl)amino}-S-{[4-(1-{[2-(1,3-dioxolan-2-yl)-3-[(4-methoxyphenyl)methoxy]phenyl]methyl}indol-3-yl)pyrimidin-2-yl]amino}-4-methoxyphenyl)acetamide (150 mg, 74.65%) as a brown solid. MS (ESI): mass calculated for C₄₄H₄₉N₇O₆ 771.37 m/z, found 772.30 [M+H]⁺.

### Step 6: Synthesis of N-(2-((2-(dimethylamino)ethyl)(methyl)amino)-S-((4-(1-(2-formyl-3-hydroxybenzyl)-1H-indol-3-yl)pyrimidin-2-yl)amino)-4-methoxyphenyl)acetamide

N-(2-((2-(dimethylamino)ethyl)(methyl)amino)-S-((4-(1-(2-formyl-3-((4-methoxybenzyl)oxy)benzyl)-1H-indol-3-yl)pyrimidin-2-yl)amino)-4-methoxyphenyl)acetamide (100 mg, 0.137 mmol), a stir bar, DCM (4 mL) was added to a 40 mL reaction vessel and stirred until homogeneous (˜10 min). Then, TFA (1.57 mg, 0.014 mmol) was added and the mixture was evacuated and backfilled with nitrogen at rt. The resulting mixture was maintained under nitrogen and stirred at rt for 1 h. The resulting mixture turned into yellow. The reaction was quenched with water (50 mL) and extracted with ethyl acetate (3×50 mL). The combined organic layer was washed with sat. NaCl (50 ml), dried over anhydrous Na₂SO₄, filtered, and concentrated to afford a white solid. It was purified by column chromatography on silica gel with (0-4%, DCM/MeOH) and preparative HPLC using Column: XSelect CSH Prep C18 OBD Column, 19*250 mm, 5 μm; Mobile Phase A: Water(0.1% FA), Mobile Phase B: MeOH—-HPLC; Flow rate: 25 mL/min; Gradient: 25% B to 55% B in 7 min, 55% B; Wave Length: 254/220 nm to afford N-(2-((2-(dimethylamino)ethyl)(methyl)amino)-S-((4-(1-(2-formyl-3-hydroxybenzyl)-1H-indol-3-yl)pyrimidin-2-yl)amino)-4-methoxyphenyl)acetamide as a yellow solid (3.3 mg, 3.89%). MS (ESI): mass calculated for C₃₄H₃₇N₇O₄ 607.29 m/z, found 608.35 [M+H]. ¹H NMR (300 MHz, DMSO-d6) δ 10.64 (s, 1H), 9.93 (s, 1H), 8.98 (s, 1H), 8.75 (s, 1H), 8.31 (dd, J=18.2, 5.0 Hz, 3H), 7.91 (s, 1H), 7.42-7.33 (m, 1H), 7.27 (dd, J=9.3, 6.5 Hz, 2H), 7.21-7.13 (m, 2H), 6.99 (s, 1H), 6.91 (d, J=8.1 Hz, 1H), 5.91 (s, 2H), 5.82 (d, J=7.7 Hz, 1H), 3.85 (s, 3H), 2.89 (t, J=5.6 Hz, 2H), 2.69 (s, 3H), 2.35 (s, 2H), 2.25 (s, 5H), 1.96 (s, 3H), 1.24 (s, 1H).

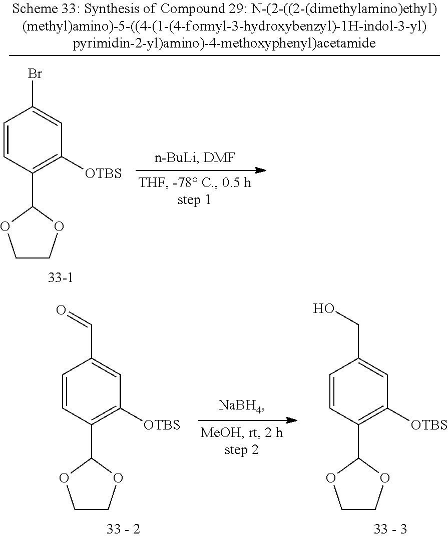 [SMILES: CC(C)(C)[Si](C)(C)Oc1cc(Br)ccc1C1OCCO1 ; CC(C)(C)[Si](C)(C)Oc1cc(C=O)ccc1C1OCCO1 ; CC(C)(C)[Si](C)(C)Oc1cc(CO)ccc1C1OCCO1]

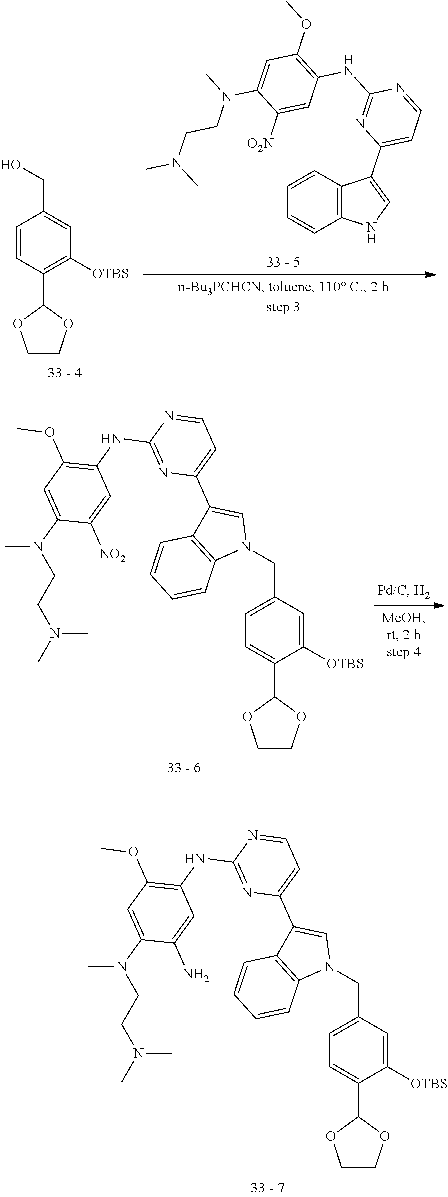 [SMILES: CC(C)(C)[Si](C)(C)Oc1cc(CO)ccc1C1OCCO1 ; COc1cc(N(C)CCN(C)C)c([N+](=O)[O-])cc1Nc1nccc(-c2c[nH]c3ccccc23)n1 ; COc1cc(N(C)CCN(C)C)c([N+](=O)[O-])cc1Nc1nccc(-c2cn(Cc3ccc(C4OCCO4)c(O[Si](C)(C)C(C)(C)C)c3)c3ccccc23)n1 ; COc1cc(N(C)CCN(C)C)c(N)cc1Nc1nccc(-c2cn(Cc3ccc(C4OCCO4)c(O[Si](C)(C)C(C)(C)C)c3)c3ccccc23)n1]

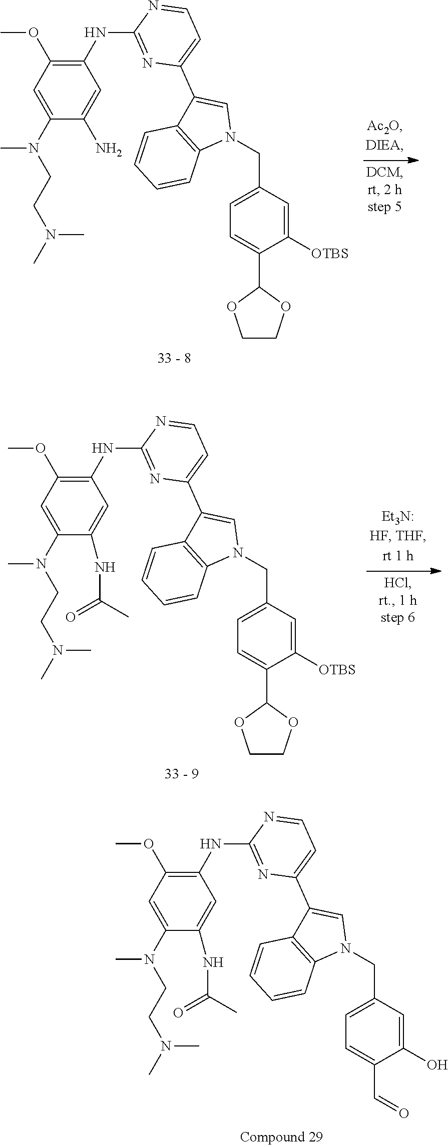 [SMILES: COc1cc(N(C)CCN(C)C)c(N)cc1Nc1nccc(-c2cn(Cc3ccc(C4OCCO4)c(O[Si](C)(C)C(C)(C)C)c3)c3ccccc23)n1 ; COc1cc(N(C)CCN(C)C)c(NC(C)=O)cc1Nc1nccc(-c2cn(Cc3ccc(C4OCCO4)c(O[Si](C)(C)C(C)(C)C)c3)c3ccccc23)n1 ; COc1cc(N(C)CCN(C)C)c(NC(C)=O)cc1Nc1nccc(-c2cn(Cc3ccc(C=O)c(O)c3)c3ccccc23)n1]

### Step 1: Synthesis of 3-((tert-butyldimethylsilyl)oxy)-4-(1,3-dioxolan-2-yl)benzaldehyde

5-bromo-2-(1,3-dioxolan-2-yl)phenoxy(tert-butyl)dimethylsilane (1 g, 2.783 mmol, 1 equiv), a stir bar, THF (12 mL) was added to a 100 mL reaction vessel under nitrogen and stirred at −78° C. (˜30 min). Then slow drops plus n-BuLi (2.5M solution in Hexanes,SpcSeal) (1.3 ml) (˜10 min). Then under nitrogen and stirred at −78° C. for 0.5 h. Then slow drops plus DMF (0.41 g, 5.566 mmol, 2 equiv) at −78° C. then, under nitrogen, stirred at −78° C. for 0.5 h. The resulting mixture turned into white. The reaction was quenched with water (50 mL). And extracted with ethyl acetate (3×50 mL). The combined organic layer was washed with sat. NaCl (50 ml), dried over anhydrous Na₂SO₄, filtered, and concentrated to afford a yellow solid. It was purified by column chromatography on silica gel with (0-19%, PE/EA) to afford 3-[(tert-butyldimethylsilyl)oxy]-4-(1,3-dioxolan-2-yl)benzaldehyde as a yellow oil (750 mg, 87.37%). MS (ESI): mass calculated for C₁₆H₂₄O₄Si 308.14 m/z, found 309.10 [M+H]⁺. Step 2: Synthesis of (3-((tert-butyldimethylsilyl)oxy)-4-(1,3-dioxolan-2-yl)phenyl)methanol 3-[(tert-butyldimethylsilyl)oxy]-4-(1,3-dioxolan-2-yl)benzaldehyde (750 mg, 2.432 mmol, 1 equiv), a stir bar, MeOH (8 mL) was added to a 40 mL reaction vessel and stirred until homogeneous (˜10 min). Then NaBH4 (183.97 mg, 4.864 mmol, 2 equiv) was added and the mixture was evacuated and backfilled with nitrogen at 0° C. The resulting mixture was maintained under nitrogen and stirred at rt for 2 h. The resulting mixture turned into white. The reaction was quenched with water (100 mL). And extracted with ethyl acetate (3×100 mL).

The combined organic layer was washed with sat. NaCl (100 ml), dried over anhydrous Na₂SO₄, filtered, and concentrated to afford a brown solid. It was purified by column chromatography on silica gel with (0-35%, PE/EA) to afford {3-[(tert-butyldimethylsilyl)oxy]-4-(1,3-dioxolan-2-yl)phenyl}methanol (600 mg, 79.48%) as a white solid. MS (ESI): mass calculated for C₁₆H₂₆O₄Si 310.16 m/z, found 311.05 [M+H].

### Step 3: Synthesis of N1-(4-(1-(3-((tert-butyldimethylsilyl)oxy)-4-(1,3-dioxolan-2-yl)benzyl)-1H-indol-3-yl)pyrimidin-2-yl)-N4-(2-(dimethylamino)ethyl)-2-methoxy-N4-methyl-S-nitrobenzene-1,4-diamine

{3-[(tert-butyldimethylsilyl)oxy]-4-(1,3-dioxolan-2-yl)phenyl}methanol (200 mg, 0.644 mmol, 1 equiv), a stir bar,Toluene (5 mL) was added to a 40 mL reaction vessel and stirred until homogeneous (˜10 min). ThenN1-[2-(dimethylamino)ethyl]-N4-[4-(1H-indol-3-yl)pyrimidin-2-yl]-S-methoxy-N1-methyl-2-nitrobenzene-1,4-diamine (445.97 mg, 0.966 mmol, 1.5 equiv) was added and the mixture was evacuated and backfilled with nitrogen at rt. The resulting mixture was maintained under nitrogen and stirred at 120° C. for 2 h. The resulting mixture turned into black. The reaction was quenched with water (50 mL). And extracted with ethyl acetate (3×50 mL). The combined organic layer was washed with sat. NaCl (50 ml), dried over anhydrous Na₂SO₄, filtered and concentrated to afford a black solid. It was purified by column chromatography on silica gel with (0-68%, PE/EA) to afford N4-{4-[1-({3-[(tert-butyldimethylsilyl)oxy]-4-(1,3-dioxolan-2-yl)phenyl}methyl) indol-3-yl]pyrimidin-2-yl}-N1-[2-(dimethylamino)ethyl]-S-methoxy-N1-methyl-2-nitrobenzene-1,4-diamine (230 mg, 47.35%) as a dark yellow solid. MS (ESI): mass calculated for C₄₀H₅₁N₇O₆Si 753.55 m/z, found 754.50 [M+H]⁺.

### Step 4: Synthesis of N4-(4-(1-(3-((tert-butyldimethylsilyl)oxy)-4-(1,3-dioxolan-2-yl)benzyl)-1H-indol-3-yl)pyrimidin-2-yl)-N1-(2-(dimethylamino)ethyl)-S-methoxy-N1-methylbenzene-1,2,4-triamine

N4-{4-[1-({3-[(tert-butyldimethylsilyl)oxy]-4-(1,3-dioxolan-2-yl)phenyl}methyl) indol-3-yl]pyrimidin-2-yl}-N1-[2-(dimethylamino)ethyl]-S-methoxy-N1-methyl-2-nitrobenzene-1,4-diamine (330 mg, 0.438 mmol, 1 equiv), a stir bar, MeOH (6 mL) was added to a 50 mL reaction vessel and stirred until homogeneous (˜10 min). Then Pd/C (186.31 mg, 1.752 mmol, 4 equiv) was added and the mixture was evacuated and backfilled with hydrogen at rt. The resulting mixture was maintained under hydrogen and stirred at rt for 2 h. The resulting mixture turned into black. filtered and concentrated to N4-{4-[1-({3-[(tert-butyldimethylsilyl)oxy]-4-(1,3-dioxolan-2-yl)phenyl}methyl) indol-3-yl]pyrimidin-2-yl}-N1-[2-(dimethylamino)ethyl]-S-methoxy-NT-methylbenzene-1,2,4-triamine (270 mg, 85.21%) as a yellow solid. MS (ESI): mass calculated for C₄₀H₅₃N₇O₄Si 723.39 m/z, found 724.50 [M+H]⁺.

### Step 5: Synthesis of N-(5-((4-(1-(3-((tert-butyldimethylsilyl)oxy)-4-(1,3-dioxolan-2-yl)benzyl)-1H-indol-3-yl)pyrimidin-2-yl)amino)-2-((2-(dimethylamino)ethyl)(methyl)amino)-4-methoxyphenyl)acetamide

N4-{4-[1-({3-[(tert-butyldimethylsilyl)oxy]-4-(1,3-dioxolan-2-yl)phenyl}methyl) indol-3-yl]pyrimidin-2-yl}-N1-[2-(dimethylamino)ethyl]-S-methoxy-N1-methylbenzene-1,2,4-triamine (260 mg, 0.359 mmol, 1 equiv), a stir bar, DCM (6 mL) was added to a 40 mL reaction vessel and stirred until homogeneous (˜10 min). Then DIEA (92.83 mg, 0.718 mmol, 2 equiv), Ac₂O (54.99 mg, 0.538 mmol, 1.5 equiv) was added and the mixture was evacuated and backfilled with nitrogen at rt. The resulting mixture was maintained under nitrogen and stirred at rt for 2 h. The resulting mixture turned into black. The reaction was quenched with water (50 mL). And extracted with ethyl acetate (3×50 mL). The combined organic layer was washed with sat. NaCl (50 ml), dried over anhydrous Na₂SO₄, filtered, and concentrated to afford a black solid. It was purified by column chromatography on silica gel with (0-75%, PE/EA) to afford N-[5-({4-[1-({3-[(tert-butyldimethylsilyl)oxy]-4-(1,3-dioxolan-2-yl)phenyl}methyl) indol-3-yl]pyrimidin-2-yl}amino)-2-{[2-(dimethylamino)ethyl](methyl)amino}-4-methoxyphenyl]acetamide (200 mg, 72.70%) as a brown oil. MS (ESI): mass calculated for C₄₂H₅₅N₇O₅Si 765.40 m/z, found 766.50 [M+H]⁺.

### Step 6: Synthesis of N-(2-((2-(dimethylamino)ethyl)(methyl)amino)-S-((4-(1-(4-formyl-3-hydroxybenzyl)-1H-indol-3-yl)pyrimidin-2-yl)amino)-4-methoxyphenyl)acetamide

N-(5-((4-(1-(3-((tert-butyldimethylsilyl)oxy)-4-(1,3-dioxolan-2-yl)benzyl)-1H-indol-3-yl)pyrimidin-2-yl)amino)-2-((2-(dimethylamino)ethyl)(methyl)amino)-4-methoxyphenyl)acetamide (160 mg, 0.209 mmol), a stir bar, THF (4 mL) was added to a 40 mL reaction vessel and stirred until homogeneous (˜10 min). Then, triethylamine trihydrofluoride (67.34 mg, 0.418 mmol) was added and the mixture was evacuated and backfilled with nitrogen at rt The resulting mixture was maintained under nitrogen and stirred at rt for 1 h. The resulting mixture turned into yellow. 2N HCl (1 mL) was added dropwise at 0° C. and stirred at rt for 1 h. the reaction mixture was concentrated to dryness to give yellow solid. The yellow solid was purified by preparative HPLC using Column: XBridge Prep OBD C18 Column, 30*150 mm, 5 μm; Mobile Phase A: Water (10 mmol/L NH₄HCO₃), Mobile Phase B: ACN; Flow rate: 60 m/min; Gradient: 17% B to 42% B in 9 min, 42% B; Wave Length: 254/220 nm to afford N-(2-((2-(dimethylamino)ethyl)(methyl)amino)-S-((4-(1-(4-formyl-3-hydroxybenzyl)-1H-indol-3-yl)pyrimidin-2-yl)amino)-4-methoxyphenyl)acetamide as an off-white solid (5.9 mg, 4.64%). MS (ESI): mass calculated for C₃₄H₃₇N₇O₄ 607.29 m/z, found 608.30 [M+H]⁺. ¹H NMR (300 MHz, DMSO-d6) δ 10.64 (s, 1H), 10.18 (s, 1H), 9.99 (s, 1H), 9.01 (s, 1H), 8.87 (s, 1H), 8.39-8.32 (m, 1H), 8.32-8.24 (m, 1H), 7.92 (s, 1H), 7.67-7.58 (m, 1H), 7.52-7.43 (m, 1H), 7.30-7.11 (m, 3H), 7.01 (s, 1H), 6.94-6.85 (m, 1H), 6.75-6.69 (m, 1H), 5.60 (s, 2H), 3.85 (s, 3H), 2.88 (t, J=5.6 Hz, 2H), 2.71 (s, 3H), 2.30 (t, J=5.8 Hz, 2H), 2.23 (s, 6H), 2.01 (s, 3H).

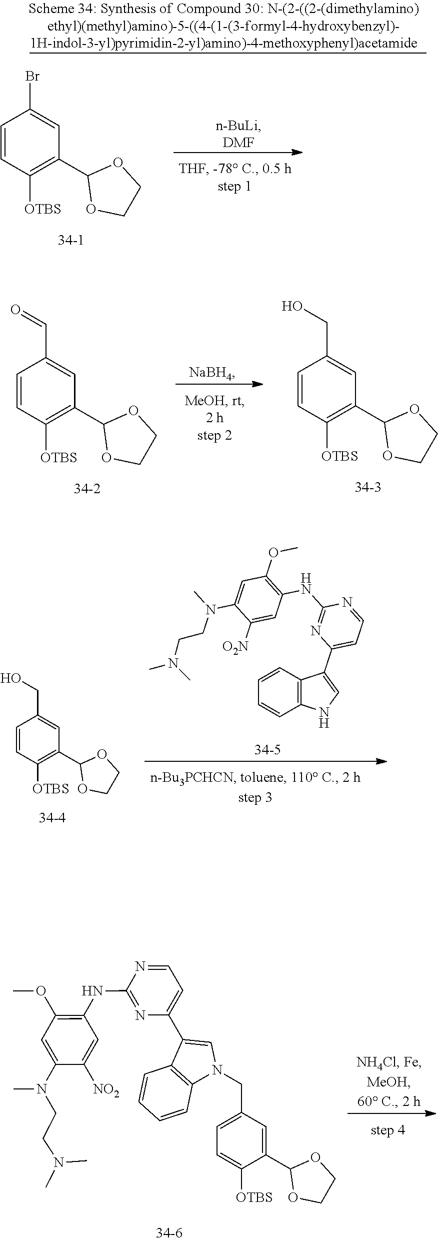 [SMILES: CC(C)(C)[Si](C)(C)Oc1ccc(Br)cc1C1OCCO1 ; CC(C)(C)[Si](C)(C)Oc1ccc(C=O)cc1C1OCCO1 ; CC(C)(C)[Si](C)(C)Oc1ccc(CO)cc1C1OCCO1 ; CC(C)(C)[Si](C)(C)Oc1ccc(CO)cc1C1OCCO1 ; COc1cc(N(C)CCN(C)C)c([N+](=O)[O-])cc1Nc1nccc(-c2c[nH]c3ccccc23)n1 ; COc1cc(N(C)CCN(C)C)c([N+](=O)[O-])cc1Nc1nccc(-c2cn(Cc3ccc(O[Si](C)(C)C(C)(C)C)c(C4OCCO4)c3)c3ccccc23)n1]

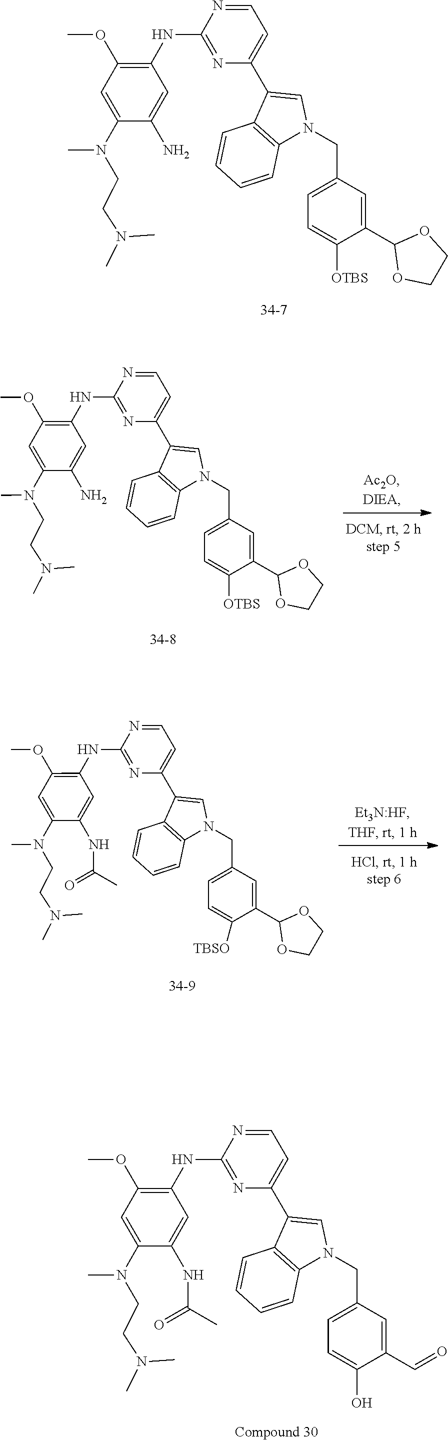 [SMILES: COc1cc(N(C)CCN(C)C)c(N)cc1Nc1nccc(-c2cn(Cc3ccc(O[Si](C)(C)C(C)(C)C)c(C4OCCO4)c3)c3ccccc23)n1 ; COc1cc(N(C)CCN(C)C)c(N)cc1Nc1nccc(-c2cn(Cc3ccc(O[Si](C)(C)C(C)(C)C)c(C4OCCO4)c3)c3ccccc23)n1 ; COc1cc(N(C)CCN(C)C)c(NC(C)=O)cc1Nc1nccc(-c2cn(Cc3ccc(O[Si](C)(C)C(C)(C)C)c(C4OCCO4)c3)c3ccccc23)n1 ; COc1cc(N(C)CCN(C)C)c(NC(C)=O)cc1Nc1nccc(-c2cn(Cc3ccc(O)c(C=O)c3)c3ccccc23)n1]

### Step 1: Synthesis of 4-((tert-butyldimethylsilyl)oxy)-3-(1,3-dioxolan-2-yl)benzaldehyde

4-bromo-2-(1,3-dioxolan-2-yl)phenoxy(tert-butyl)dimethylsilane (1 g, 2.783 mmol, 1 equiv), a stir bar, THF (15 mL) was added to a 100 mL reaction vessel under nitrogen and stirred at −78° C. (˜30 min). Then slow drops plus n-BuLi (2.5M solution in Hexanes,SpcSeal) (1.3 ml) (˜10 min). Then under nitrogen and stirred at −78° C. for 0.5 h. Then slow drops plus DMF (0.41 g, 5.566 mmol, 2 equiv) at −78° C. Then, under nitrogen, and stirred at −78° C. for 0.5 h. The resulting mixture turned into white. The reaction was quenched with water (50 mL). And extracted with ethyl acetate (3×50 mL). The combined organic layer was washed with sat. NaCl (50 ml), dried over anhydrous Na₂SO₄, filtered, and concentrated to afford a yellow solid. It was purified by column chromatography on silica gel with (0-18%, PE/EA) to afford 4-[(tert-butyldimethylsilyl)oxy]-3-(1,3-dioxolan-2-yl)benzaldehyde as a yellow oil (800 mg, 93.20%). MS (ESI): mass calculated for C₁₆H₂₄O₄Si 308.14 m/z, found 309.00 [M+H]⁺.

### Step 2: Synthesis of (4-((tert-butyldimethylsilyl)oxy)-3-(1,3-dioxolan-2-yl)phenyl)methanol

4-[(tert-butyldimethylsilyl)oxy]-3-(1,3-dioxolan-2-yl)benzaldehyde (800 mg, 2.594 mmol, 1 equiv), a stir bar, MeOH (8 mL) was added to a 40 mL reaction vessel and stirred until homogeneous (˜10 min). Then NaBH₄ (196.23 mg, 5.188 mmol, 2 equiv) was added and the mixture was evacuated and backfilled with nitrogen at 0° C. The resulting mixture was maintained under nitrogen and stirred at rt for 2 h. The resulting mixture turned into yellow. the reaction was quenched with water (100 mL). And extracted with ethyl acetate (3×100 mL).

The combined organic layer was washed with sat. NaCl (100 ml), dried over anhydrous Na₂SO₄, filtered and concentrated to afford a yellow solid. It was purified by column chromatography on silica gel with (0-42%, PE/EA) to afford {4-[(tert-butyldimethylsilyl)oxy]-3-(1,3-dioxolan-2-yl)phenyl}methanol (620 mg, 77.00%) as a light yellow oil. MS (ESI): mass calculated for C₁₆H₂₆O₄Si 310.16 m/z, found 311.10 [M+H].

### Step 3: Synthesis of N1-(4-(1-(4-((tert-butyldimethylsilyl)oxy)-3-(1,3-dioxolan-2-yl)benzyl)-1H-indol-3-yl)pyrimidin-2-yl)-N4-(2-(dimethylamino)ethyl)-2-methoxy-N4-methyl-S-nitrobenzene-1,4-diamine

4-[(tert-butyldimethylsilyl)oxy]-3-(1,3-dioxolan-2-yl)phenylmethanol (200 mg, 0.644 mmol, 1 equiv) a stir bar, toluene (5 mL) was added to a 40 mL reaction vessel and stirred until homogeneous (˜10 min). Then N1-[2-(dimethylamino)ethyl]-N4-[4-(1H-indol-3-yl)pyrimidin-2-yl]-S-methoxy-N1-methyl-2-nitrobenzene-1,4-diamine (445.97 mg, 0.966 mmol, 1.5 equiv), 2-(tributyl-lambda5-phosphanylidene)acetonitrile (310.96 mg, 1.288 mmol, 2 equiv) was added and the mixture was evacuated and backfilled with nitrogen at rt. The resulting mixture was maintained under nitrogen and stirred at 120° C. for 2 h. The resulting mixture turned into black. The reaction was quenched with water (50 mL) and extracted with ethyl acetate (3×50 mL). The combined organic layer was washed with sat. NaCl (50 ml), dried over anhydrous Na₂SO₄, filtered, and concentrated to afford a black solid. It was purified by column chromatography on silica gel with (0-73%, PE/EA) to afford N4-{4-[1-({4-[(tert-butyldimethylsilyl)oxy]-3-(1,3-dioxolan-2-yl)phenyl}methyl) indol-3-yl]pyrimidin-2-yl}-N1-[2-(dimethylamino)ethyl]-S-methoxy-N1-methyl-2-nitrobenzene-1,4-diamine (240 mg, 49.41%) as a red solid. MS (ESI): mass calculated for C₄₀H₅₁N₇O₆Si 753.37 m/z, found 754.30 [M+H]⁺.

### Step 4: Synthesis of N4-(4-(1-(4-((tert-butyldimethylsilyl)oxy)-3-(1,3-dioxolan-2-yl)benzyl)-1H-indol-3-yl)pyrimidin-2-yl)-N1-(2-(dimethylamino)ethyl)-S-methoxy-N1-methylbenzene-1,2,4-triamine

N4-{4-[1-({4-[(tert-butyldimethylsilyl)oxy]-3-(1,3-dioxolan-2-yl)phenyl}methyl) indol-3-yl]pyrimidin-2-yl}-N1-[2-(dimethylamino)ethyl]-S-methoxy-N1-methyl-2-nitrobenzene-1,4-diamine (210 mg, 0.279 mmol, 1 equiv), a stir bar, MeOH (6 mL),H₂O (2 mL) was added to a 40 mL reaction vessel and stirred until homogeneous (˜10 min). Then Fe (124.43 mg, 2.232 mmol, 8 equiv), NH₄C₁ (119.19 mg, 2.232 mmol, 8 equiv) was added and the mixture was evacuated and backfilled with nitrogen at rt. The resulting mixture was maintained under nitrogen and stirred at 60° C. for 2 h. The resulting mixture turned into black. filtered and concentrated to N4-{4-[1-({4-[(tert-butyldimethylsilyl)oxy]-3-(1,3-dioxolan-2-yl)phenyl}methyl) indol-3-yl]pyrimidin-2-yl}-N1-[2-(dimethylamino)ethyl]-S-methoxy-N1-methylbenzene-1,2,4-triamine (190 mg, 94.22%) as a brown solid. MS (ESI): mass calculated for C₄₀H₅₃N₇O₄Si 723.39 m/z, found 724.50 [M+H]⁺.

### Step 5: Synthesis of N-(5-((4-(1-(4-((tert-butyldimethylsilyl)oxy)-3-(1,3-dioxolan-2-yl)benzyl)-1H-indol-3-yl)pyrimidin-2-yl)amino)-2-((2-(dimethylamino)ethyl)(methyl)amino)-4-methoxyphenyl)acetamide

N4-{4-[1-({4-[(tert-butyldimethylsilyl)oxy]-3-(1,3-dioxolan-2-yl)phenyl}methyl) indol-3-yl]pyrimidin-2-yl}-N1-[2-(dimethylamino)ethyl]-S-methoxy-N1-methylbenzene-1,2,4-triamine (200 mg, 0.276 mmol, 1 equiv), a stir bar, DCM (6 mL) was added to a 40 mL reaction vessel and stirred until homogeneous (˜10 min). Then DIEA (107.11 mg, 0.828 mmol, 3 equiv), Ac₂O (56.40 mg, 0.552 mmol, 2 equiv) was added and the mixture was evacuated and backfilled with nitrogen at rt. The resulting mixture was maintained under nitrogen and stirred at rt for 2 h. The resulting mixture turned into black. the reaction was quenched with water (50 mL) and extracted with ethyl acetate (3×50 mL). The combined organic layer was washed with sat. NaCl (50 ml), dried over anhydrous Na₂SO₄, filtered and concentrated to afford a black solid. It was purified by column chromatography on silica gel with (0-81%, PE/EA) to afford N-[5-({4-[1-({4-[(tert-butyldimethylsilyl)oxy]-3-(1,3-dioxolan-2-yl)phenyl}methyl) indol-3-yl]pyrimidin-2-yl}amino)-2-{[2-(dimethylamino)ethyl](methyl)amino}-4-methoxyphenyl]acetamide (110 mg, 51.98%) as a light yellow oil. MS (ESI): mass calculated for C₄₂H₅₅N₇O₅Si 765.40 m/z, found 766.50 [M+H]⁺.

### Step 6: Synthesis of N-(2-((2-(dimethylamino)ethyl)(methyl)amino)-S-((4-(1-(3-formyl-4-hydroxy benzyl)-1H-indol-3-yl)pyrimidin-2-yl)amino)-4-methoxyphenyl)acetamide

N-(5-((4-(1-(4-((tert-butyldimethylsilyl)oxy)-3-formylbenzyl)-1H-indol-3-yl)pyrimidin-2-yl)amino)-2-((2-(dimethylamino)ethyl)(methyl)amino)-4-methoxyphenyl)acetamide (100 mg, 0.139 mmol), a stir bar, THF (4 mL) was added to a 40 mL reaction vessel and stirred until homogeneous (˜10 min). Then, triethylamine trihydrofluoride (33.49 mg, 0.209 mmol) was added and the mixture was evacuated and backfilled with nitrogen at rt The resulting mixture was maintained under nitrogen and stirred at rt for 1 h. The resulting mixture turned yellow. 2N HCl (1 mL) was added dropwise at 0° C. and stirred at rt for 1 h. the reaction mixture was concentrated to dryness to give yellow solid. The yellow solid was purified by preparative HPLC using Column: XBridge Shield RP18 OBD Column, 30*150 mm, 5 μm; Mobile Phase A: Water (0.05% TFA ), Mobile Phase B: ACN; Flow rate: 60 mL/min; Gradient: 14% B to 34% B in 10 min, 34% B; Wave Length: 254/220 nm to afford N-(2-((2-(dimethylamino)ethyl)(methyl)amino)-S-((4-(1-(3-formyl-4-hydroxybenzyl)-1H-indol-3-yl)pyrimidin-2-yl)amino)-4-methoxyphenyl)acetamide as a yellow solid (9.9 mg, 9.56%). MS (ESI): mass calculated for C₃₄H₃₇N₇O₄ 607.29 m/z, found 608.30 [M+H]⁺. ¹H NMR (300 MHz, DMSO-d6) δ 10.79 (s, 1H), 10.24 (s, 1H), 9.61 (s, 1H), 9.42 (s, 1H), 8.84 (s, 1H), 8.51 (s, 1H), 8.31 (d, J=5.8 Hz, 2H), 7.69-7.57 (m, 2H), 7.53 (dd, J=8.6, 2.4 Hz, 1H), 7.34 (d, J=5.9 Hz, 1H), 7.20 (dt, J=22.2, 7.2 Hz, 2H), 7.04-6.94 (m, 2H), 5.50 (s, 2H), 3.87 (s, 3H), 3.30 (s, 4H), 2.81 (d, J=4.5 Hz, 6H), 2.65 (s, 3H), 2.14 (s, 3H). 19F NMR (282 MHz, DMSO-d6) δ-74.01.

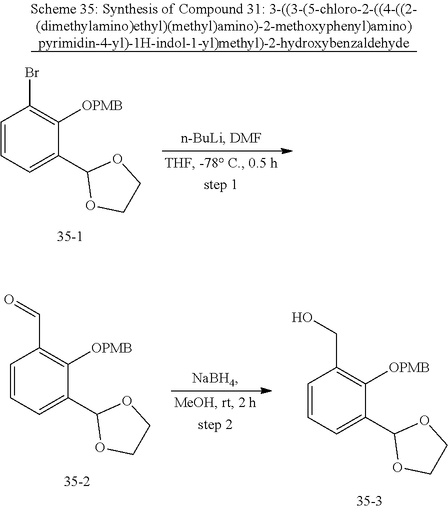

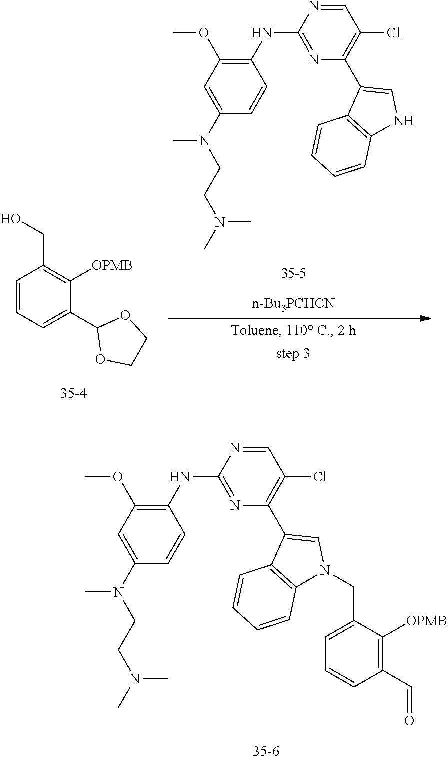

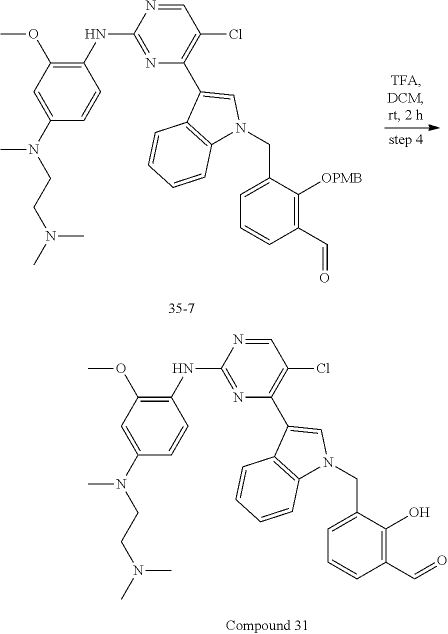

### Step 1: Synthesis of 3-(1,3-dioxolan-2-yl)-2-((4-methoxybenzyl)oxy)benzaldehyde

2-{3-bromo-2-[(4-methoxyphenyl)methoxy]phenyl}-1,3-dioxolane (1 g, 2.738 mmol, 1 equiv), a stir bar, THF (15 mL) was added to a 100 mL reaction vessel under nitrogen and stirred at −78° C. (˜30 min). Then slow drops plus n-BuLi (2.5M solution in Hexanes, SpcSeal) (1.0 ml) (˜10 min). Then under nitrogen and stirred at −78° C. for 0.5 h. Then slow drops plus DMF (0.30 g, 4.107 mmol, 1.5 equiv) at −78° C. Then under nitrogen and stirred at −78° C. for 0.5 h. The resulting mixture turned into white. the reaction was quenched with water (50 mL). And extracted with ethyl acetate (3×50 mL). The combined organic layer was washed with sat. NaCl (50 ml), dried over anhydrous Na₂SO₄, filtered and concentrated to afford a yellow solid. It was purified by column chromatography on silica gel with (0-28%, PE/EA) to afford 3-[(tert-butyldimethylsilyl)oxy]-4-(1,3-dioxolan-2-yl)benzaldehyde3-(1,3-dioxolan-2-yl)-2-[(4-methoxyphenyl)methoxy]benzaldehyde (660 mg, 76.68%) as a light yellow oil. MS (ESI): mass calculated for C18H₁₈O₅ 314.12 m/z, found 315.20 [M+H]⁺.

### Step 2: Synthesis of (3-(1,3-dioxolan-2-yl)-2-((4-methoxybenzyl)oxy)phenyl)methanol

3-(1,3-dioxolan-2-yl)-2-[(4-methoxyphenyl)methoxy]benzaldehyde (660 mg, 2.100 mmol, 1 equiv), a stir bar, MeOH (8 mL) was added to a 40 mL reaction vessel and stirred until homogeneous (˜10 min). Then, NaBH4 (158.86 mg, 4.200 mmol, 2 equiv) was added and the mixture was evacuated and backfilled with nitrogen at 0° C. The resulting mixture was maintained under nitrogen and stirred at rt for 2 h. The resulting mixture turned into white. the reaction was quenched with water (30 mL). And extracted with ethyl acetate (3×30 mL). The combined organic layer was washed with sat. NaCl (100 ml), dried over anhydrous Na₂SO₄, filtered, and concentrated to afford a brown solid. It was purified by column chromatography on silica gel with (0-29%, PE/EA) to afford [3-(1,3-dioxolan-2-yl)-2-[(4-methoxyphenyl)methoxy]phenyl]methanol (600 mg, 90.33%) as a yellow oil. MS (ESI): mass calculated for C18H₂₀O₅ 316.13 m/z, found 339.00 [M+Na]⁺.

### Step 3: Synthesis of 3-((3-(5-chloro-2-((4-((2-(dimethylamino)ethyl)(methyl)amino)-2-methoxyphenyl) amino)pyrimidin-4-yl)-1H-indol-1-yl)methyl)-2-((4-methoxybenzyl)oxy)benzaldehyde

[3-(1,3-dioxolan-2-yl)-2-[(4-methoxyphenyl)methoxy]phenyl]methanol (200 mg, 0.632 mmol, 1 equiv), a stir bar, toluene (5 mL) was added to a 40 mL reaction vessel and stirred until homogeneous (˜10 min). Then N4-[5-chloro-4-(1H-indol-3-yl)pyrimidin-2-yl]-N1-[2-(dimethylamino)ethyl]-3-methoxy-N1-methylbenzene-1,4-diamine (570.21 mg, 1.264 mmol, 2 equiv), 2-(tributyl-lambda5-phosphanylidene)acetonitrile (381.47 mg, 1.580 mmol, 2.5 equiv) was added and the mixture was evacuated and backfilled with nitrogen at rt. The resulting mixture was maintained under nitrogen and stirred at 120° C. for 2 h. The resulting mixture turned into black. The reaction was quenched with water (50 mL). And extracted with ethyl acetate (3×50 mL). The combined organic layer was washed with sat. NaCl (50 ml), dried over anhydrous Na₂SO₄, filtered and concentrated to afford a black solid. It was purified by column chromatography on silica gel with (0-74%, PE/EA) to afford 3-[(3-{5-chloro-2-[(4-{[2-(dimethylamino)ethyl](methyl)amino}-2-methoxyphenyl)amino]pyrimidin-4-yl}indol-1-yl)methyl]-2-[(4-methoxyphenyl)methoxy]benzaldehyde (350 mg, 78.50%) as a yellow solid.

MS (ESI): mass calculated for C₄₀H₄₁ClN₆O₄ 704.29 m/z, found 705.30 [M+H]⁺.

### Step 4: Synthesis of 3-((3-(5-chloro-2-((4-((2-(dimethylamino)ethyl)(methyl)amino)-2-methoxyphenyl) amino)pyrimidin-4-yl)-1H-indol-1-yl)methyl)-2-hydroxybenzaldehyde

3-((3-(5-chloro-2-((4-((2-(dimethylamino)ethyl)(methyl)amino)-2-methoxyphenyl)amino)pyrimidin-4-yl)-1H-indol-1-yl)methyl)-2-((4-methoxybenzyl)oxy)benzaldehyde (100 mg, 0.142 mmol), a stir bar, DCM (4 mL) was added to a 40 mL reaction vessel and stirred until homogeneous (˜10 min). Then TFA (1.62 mg, 0.014 mmol) was added and the mixture was evacuated and backfilled with nitrogen at rt. The resulting mixture was maintained under nitrogen and stirred at rt for 2 h.. The resulting mixture turned into yellow. the reaction was quenched with water (50 mL). And extracted with ethyl acetate (3×50 mL). The combined organic layer was washed with sat. NaCl (50 ml), dried over anhydrous Na₂SO₄, filtered, and concentrated to afford a white solid. It was purified by column chromatography on silica gel with (0-4%, DCM/MeOH) and preparative HPLC using Column: XSelect CSH Prep C18 OBD Column, 19*250 mm, 5 μm; Mobile Phase A: Water(0.05% TFA ), Mobile Phase B: MeOH—-HPLC; Flow rate: 25 mL/min; Gradient: 50% B to 80% B in 7 min, 80% B; Wave Length: 254/220 nm to afford 3-((3-(5-chloro-2-((4-((2-(dimethylamino) ethyl)(methyl)amino)-2-methoxyphenyl)amino)pyrimidin-4-yl)-1H-indol-1-yl)methyl)-2-hydroxybenzaldehyde as an yellow solid (6.0 mg, 5.65%). MS (ESI): mass calculated for C₃₄H₃₄ClN₆O₃ 584.23 m/z, found 585.20 [M+H]⁺. ¹H NMR (300 MHz, DMSO-d₆) δ 11.37 (s, 1H), 10.06 (s, 1H), 9.59 (s, 1H), 8.70 (s, 1H), 8.33 (d, J=12.2 Hz, 2H), 8.32 (s, 1H), 7.73 (dd, J=7.7, 1.8 Hz, 1H), 7.53 (d, J=8.3 Hz, 1H), 7.38 (d, J=8.6 Hz, 1H), 7.32 (dd, J=7.5, 1.7 Hz, 1H), 7.27-7.14 (m, 1H), 7.07-6.94 (m, 1H), 6.48 (d, J=2.6 Hz, 1H), 6.41 (dd, J=8.7, 2.6 Hz, 1H), 5.57 (s, 2H), 3.79 (s, 3H), 3.69 (t, J=7.3 Hz, 2H), 3.27 (s, 2H), 2.97 (s, 3H), 2.86 (d, J=4.2 Hz, 6H). ¹⁹F NMR (282 MHz, DMSO-d₆) δ-74.28 (d, J=2.2 Hz).

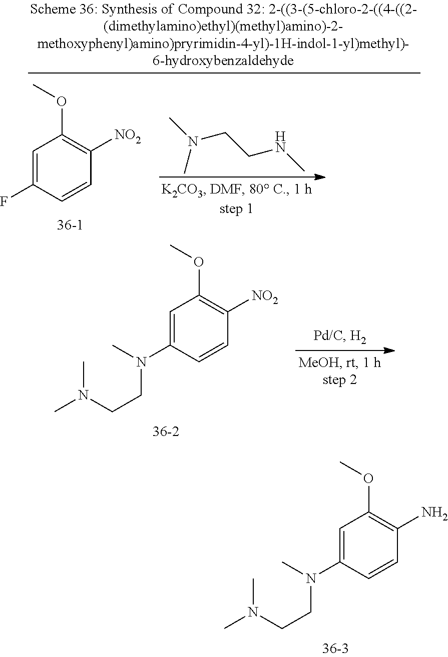

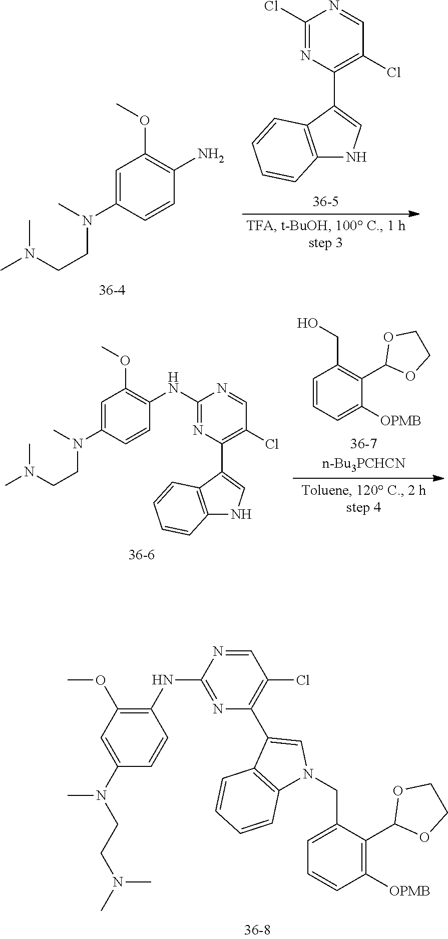

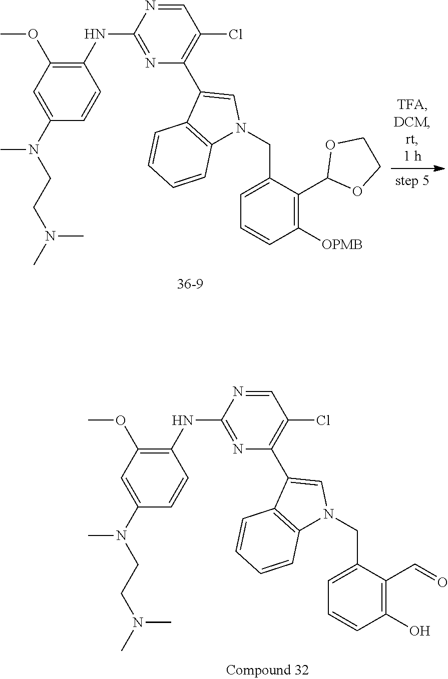

### Step 1: Synthesis of N1-(3-methoxy-4-nitrophenyl)-N1,N2,N2-trimethylethane-1,2-diamine

4-fluoro-2-methoxy-1-nitrobenzene (1.5 g, 8.765 mmol, 1 equiv), [2-(dimethylamino)ethyl](methyl)amine (1.34 g, 13.148 mmol, 1.5 equiv), K₂CO₃ (2.42 g, 17.530 mmol, 2.0 equiv), DMF (10 mL, 129.215 mmol, 14.74 equiv) were added to a 100 mL sized reaction vessel. The reaction mixture was stirred at 80° C. for 1 hours under N₂. The reaction was quenched with water (50 mL). The resulting mixture was extracted with ethyl acetate (3×50 mL). The organic layers were combined, dried over anhydrous sodium sulfate, filtered, and concentrated. The residue was purified by silica gel column chromatography, eluted with EA/PE (0-30% o) to afford N1-(3-methoxy-4-nitrophenyl)-N1, N2, N2-trimethylethane-1,2-diamine (2.1 g, 94.58% o) as a yellow solid. MS (ESI) calculated for C₁₂H₁₉N₃O₃, 253.14 m/z, found: 254.15[M+H].

### Step 2: Synthesis of N-(2-(dimethylamino)ethyl)-3-methoxy-N1-methylbenzene-1,4-diamine

Synthesis of N-(3-methoxy-4-nitrophenyl)-N1,N2,N2-trimethylethane-1,2-diamine (1.0 g, 3.948 mmol, 1 equiv), Pd/C were added to a 100 mL sized reaction vessel. The reaction mixture was stirred at rt for 1 hours under H₂. The reaction was quenched with water (50 mL). The resulting mixture was extracted with ethyl acetate (3×50 mL). The organic layers were combined, dried over anhydrous sodium sulfate, filtered and concentrated, The residue was purified by silica gel column chromatography, eluted with EA/PE (0-30%) to afford NT-[2-(dimethylamino)ethyl]-3-methoxy-N1-methylbenzene-1,4-diamine (1 g, 113.43%.) as a yellow solid. MS (ESI) calculated for C₁₂H₂₁N₃O_{, 223.17} m/z, found: 224.10[M+H]⁺.

### Step 3: Synthesis of N1-(5-chloro-4-(1H-indol-3-yl)pyrimidin-2-yl)-N4-(2-(dimethylamino)ethyl)-2-methoxy-N4-methylbenzene-1,4-diamine

N1-[2-(dimethylamino)ethyl]-3-methoxy-N1-methylbenzene-1,4-diamine (1 g, 4.478 mmol, 1 equiv), 3-(2,5-dichloropyrimidin-4-yl)-1H-indole (1.18 g, 4.478 mmol, 1 equiv), TFA (1.02 g, 8.956 mmol, 2.0 equiv), t-BuOH (10 mL, 105.230 mmol, 23.50 equiv) were added to a 100 mL sized reaction vessel. The reaction mixture was stirred at 100° C. for 1 hours under N₂. The reaction was quenched with water (50 mL). The resulting mixture was extracted with ethyl acetate (3×50 mL). The organic layers were combined, dried over anhydrous sodium sulfate, filtered and concentrated, The residue was purified by silica gel column chromatography, eluted with EA/PE (0-100%) to afford N1-(5-chloro-4-(1H-indol-3-yl)pyrimidin-2-yl)-N4-(2-(dimethylamino)ethyl)-2-methoxy-N4-methylbenzene-1,4-diamine (1 g, 49.52%) as a yellow solid. MS (ESI) calculated for C₂₄H₂₇ClN₆O, 450.19 m/z, found: 451.15[M+H]⁺.

### Step 4: Synthesis of 2-((3-(5-chloro-2-((4-((2-(dimethylamino)ethyl)(methyl)amino)-2-methoxyphenyl)amino)pyrimidin-4-yl)-1H-indol-1-yl)methyl)-6-((4-methoxybenzyl)oxy)benzaldehyde

N4-[5-chloro-4-(1H-indol-3-yl)pyrimidin-2-yl]-N1-[2-(dimethylamino)ethyl]-3-methoxy-N1-methylbenzene-1,4-diamine (200 mg, 0.443 mmol, 1 equiv), a stir bar, Toluene (5 mL) was added to a 40 mL reaction vessel and stirred until homogeneous (˜10 min). Then [2-(1,3-dioxolan-2-yl)-3-[(4-methoxyphenyl)methoxy]phenyl]methanol(280.60 mg, 0.886 mmol, 2 equiv), 2-(tributyl-lambda5-phosphanylidene)acetonitrile (267.60 mg, 1.107 mmol, 2.5 equiv), was added and the mixture was evacuated and backfilled with nitrogen at rt. The resulting mixture was maintained under nitrogen and stirred at 120° C. for 2 h. The resulting mixture turned into black. the reaction was quenched with water (50 mL). And extracted with ethyl acetate (3×50 mL). The combined organic layer was washed with sat. NaCl (50 ml), dried over anhydrous Na₂SO₄, filtered and concentrated to afford a black solid. It was purified by column chromatography on silica gel with (0-84%, PE/EA) to afford 2-[(3-{5-chloro-2-[(4-{[2-(dimethylamino)ethyl](methyl)amino}-2-methoxyphenyl)amino]pyrimidin-4-yl}indol-1-yl)methyl]-6-[(4-methoxyphenyl)methoxy]benzaldehyde (200 mg, 63.94%) as a yellow solid.

MS (ESI): mass calculated for C₄₀H₄₁ClN₆O₄ 704.29 m/z, found 705.25 [M+H]⁺.

### Step 5: Synthesis of 2-((3-(5-chloro-2-((4-((2-(dimethylamino)ethyl)(methyl)amino)-2-methoxyphenyl)amino)pyrimidin-4-yl)-1H-indol-1-yl)methyl)-6-hydroxybenzaldehyde

2-((3-(5-chloro-2-((4-((2-(dimethylamino)ethyl)(methyl)amino)-2-methoxyphenyl)amino)pyrimidin-4-yl)-1H-indol-1-yl)methyl)-6-((4-methoxybenzyl)oxy)benzaldehyde (100 mg, 0.142 mmol), a stir bar, DCM (4 mL) was added to a 40 mL reaction vessel and stirred until homogeneous (˜10 min). Then TFA (1.62 mg, 0.014 mmol) was added, and the mixture was evacuated and backfilled with nitrogen at rt. The resulting mixture was maintained under nitrogen and stirred at rt for 1 h. The resulting mixture turned into yellow. the reaction mixture was concentrated to dryness to give yellow solid. The yellow solid was purified by preparative HPLC using Column: Xselect CSH C18 OBD Column 30*150 mm 5 μm, n; Mobile Phase A: Water(0.05% TFA ), Mobile Phase B: ACN; Flow rate: 60 m/min; Gradient: 29% B to 47% B in 8 min, 47% B; Wave Length: 254/220 nm to afford 2-((3-(5-chloro-2-((4-((2-(dimethylamino)ethyl)(methyl)amino)-2-methoxyphenyl)amino)pyrimidin-4-yl)-1H-indol-1-yl)methyl)-6-hydroxybenzaldehyde as a yellow solid (12 mg, 12.05%). MS (ESI): mass calculated for C₃₂H₃₃ClN₆O₃ 584.23 m/z, found 585.25 [M+H]⁺. ¹H NMR (300 MHz, DMSO-d6) δ 11.10 (s, 1H), 10.60 (s, 1H), 9.72 (s, 1H), 8.63 (s, 1H), 8.34 (d, J=18.2 Hz, 3H), 7.44-7.25 (m, 3H), 7.17 (t, J=7.5 Hz, 1H), 7.01 (t, J=7.6 Hz, 1H), 6.93 (d, J=8.3 Hz, 1H), 6.50 (d, J=2.6 Hz, 1H), 6.43 (dd, J=8.8, 2.6 Hz, 1H), 5.91 (d, J=6.6 Hz, 3H), 3.80 (s, 3H), 3.70 (t, J=7.4 Hz, 2H), 3.29 (t, J=6.5 Hz, 1H), 2.98 (s, 3H), 2.87 (d, J=3.9 Hz, 6H). 19F NMR (282 MHz, DMSO-d6) δ-74.17.

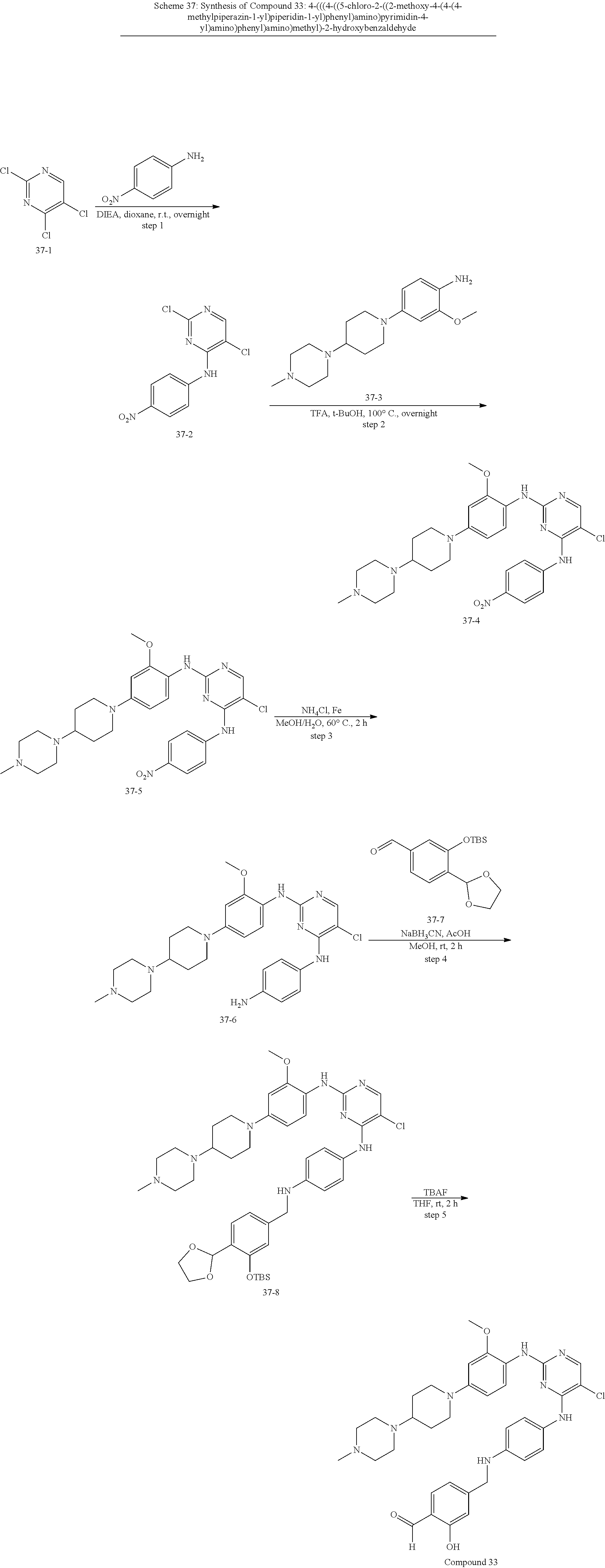

### Step 1: Synthesis of 2,5-dichloro-N-(4-nitrophenyl)pyrimidin-4-amine

2,4,5-trichloropyrimidine (2 g, 10.904 mmol, 1 equiv), a stir bar, p-nitroaniline (1.51 g, 10.904 mmol, 1 equiv), DIEA (629.67 mg, 4.870 mmol, 5 equiv), NaH (0.70 g, 17.446 mmol, 1.6 equiv, 60%) and DMF (15 mL) were added to a 100 mL reaction vessel and stirred until homogeneous. The reaction mixture was stirred for overnight at rt under N₂. The reaction was filtered and concentrated to dryness to give 2,5-dichloro-N-(4-nitrophenyl)pyrimidin-4-amine (2 g, 64.34%) as yellow solid. MS (ESI): mass calculated for C₁₀H₆Cl₂N₄O₂ 283.99 m/z, found 285.10 [M+H]⁺.

### Step 2: Synthesis of 5-chloro-N2-(2-methoxy-4-(4-(4-methylpiperazin-1-yl)piperidin-1-yl)phenyl)-N4-(4-nitrophenyl)pyrimidine-2,4-diamine

2,5-dichloro-N-(4-nitrophenyl)pyrimidin-4-amine (500 mg, 1.754 mmol, 1 equiv), a stir bar, 2-methoxy-4-[4-(4-methylpiperazin-1-yl)piperidin-1-yl]aniline (533.95 mg, 1.754 mmol, 1 equiv), TFA (299.98 mg, 2.631 mmol, 1.5 equiv) and t-BuOH (3 mL, 31.569 mmol, 18.00 equiv) were added to a 100 mL reaction vessel and stirred until homogeneous. The reaction mixture was stirred for overnight at 100° C. under N₂. The reaction was filtered and concentrated to dryness to give 5-chloro-N2-{2-methoxy-4-[4-(4-methylpiperazin-1-yl)piperidin-1-yl]phenyl}-N4-(4-nitrophenyl)pyrimidine-2,4-diamine (870 mg, 89.69%) as light green solid. MS (ESI): mass calculated for C₂₇H₃₃ClN₈O₃ 552.24 m/z, found 553.30 [M+H]⁺.

### Step 3: Synthesis of N4-(4-aminophenyl)-S-chloro-N2-(2-methoxy-4-(4-(4-methylpiperazin-1-yl)piperidin-1-yl)phenyl)pyrimidine-2,4-diamine

5-chloro-N2-{2-methoxy-4-[4-(4-methylpiperazin-1-yl)piperidin-1-yl]phenyl}-N4-(4-nitrophenyl)pyrimidine-2,4-diamine (1 g, 1.808 mmol, 1 equiv), a stir bar, NH₄Cl (0.48 g, 9.040 mmol, 5 equiv), Fe (1.01 g, 18.080 mmol, 10 equiv) and MeOH/H₂₀(5:1) 25 ml were added to a 100 mL reaction vessel and stirred until homogeneous. The reaction mixture was stirred for 2 h at 60° C. under N₂. The reaction was filtered and concentrated to dryness to give N4-(4-aminophenyl)-S-chloro-N2-{2-methoxy-4-[4-(4-methylpiperazin-1-yl)piperidin-1-yl]phenyl}pyrimidine-2,4-diamine (800 mg, 84.59%) as a yellow solid. MS (ESI): mass calculated for C₂₇H₃₅ClN₈O 522.26 m/z, found 523.30 [M+H]⁺.

### Step 4: Synthesis of N4-(4-((3-((tert-butyldimethylsilyl)oxy)-4-(1,3-dioxolan-2-yl)benzyl)amino)phenyl)-S-chloro-N2-(2-methoxy-4-(4-(4-methylpiperazin-1-yl)piperidin-1-yl)phenyl)pyrimidine-2,4-diamine

N4-(4-aminophenyl)-S-chloro-N2-(2-methoxy-4-(4-(4-methylpiperazin-1-yl)piperidin-1-yl)phenyl)pyrimidine-2,4-diamine (100 mg, 0.191 mmol), a stir bar, MeOH (4 mL) was added to a 40 mL reaction vessel and stirred until homogeneous (˜10 min). Then 3-((tert-butyldimethylsilyl)oxy)-4-(1,3-dioxolan-2-yl)benzaldehyde (88.45 mg, 0.286 mmol), AcOH (1.15 mg, 0.019 mmol), NaBH₃CN (36.04 mg, 0.573 mmol) was added and the mixture was evacuated and backfilled with nitrogen at rt. The resulting mixture was maintained under nitrogen and stirred at r.t for 2 h. The resulting mixture turned into white. The reaction was quenched with water (50 mL). And extracted with ethyl acetate (3×50 mL). The combined organic layer was washed with sat. NaCl (50 ml), dried over anhydrous Na₂SO₄, filtered and concentrated to afford a white solid. It was purified by column chromatography on silica gel with (0-7%, DCM/MeOH) to afford N4-(4-((3-((tert-butyldimethylsilyl)oxy)-4-(1,3-dioxolan-2-yl)benzyl)amino)phenyl)-S-chloro-N2-(2-methoxy-4-(4-(4-methylpiperazin-1-yl)piperidin-1-yl)phenyl)pyrimidine-2,4-diamine as a light yellow oil (90 mg, 57.73%). MS (ESI): mass calculated for C₄₃H₅₉ClN₈O₄Si 814.41 m/z, found 815.30 [M+H]⁺.

### Step 5: Synthesis of 4-(((4-((5-chloro-2-((2-methoxy-4-(4-(4-methylpiperazin-1-yl)piperidin-1-yl)phenyl) amino)pyrimidin-4-yl)amino)phenyl)amino)methyl)-2-hydroxybenzaldehyde

N4-(4-((3-((tert-butyldimethylsilyl)oxy)-4-(1,3-dioxolan-2-yl)benzyl)amino)phenyl)-S-chloro-N2-(2-methoxy-4-(4-(4-methylpiperazin-1-yl)piperidin-1-yl)phenyl)pyrimidine-2,4-diamine (90 mg, 0.110 mmol), a stir bar, THF (3 mL) was added to a 40 mL reaction vessel and stirred until homogeneous (˜10 min). Then tetrabutylammonium fluoride (43.28 mg, 0.165 mmol) was added and the mixture was evacuated and backfilled with nitrogen at 0° C. and stirred at rt for 2 h. The resulting mixture turned into yellow. the reaction mixture was concentrated to dryness to give yellow solid. The yellow solid was purified by preparative HPLC using Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 67% B in 9 min, 67% B; Wave Length: 254/220 nm; to afford 4-(((4-((5-chloro-2-((2-methoxy-4-(4-(4-methylpiperazin-1-yl)piperidin-1-yl)phenyl)amino)pyrimidin-4-yl)amino)phenyl)amino)methyl)-2-hydroxybenzaldehyde as a light yellow (2.5 mg, 3.45%). MS (ESI): mass calculated for C₃₅H₄₁ClN₈O₃ 656.30 m/z, found 657.25 [M+H]⁺. ¹H NMR (300 MHz, DMSO-d6) δ 10.19 (s, 1H), 8.44 (s, 1H), 7.94 (s, 1H), 7.60 (dd, J=10.9, 8.3 Hz, 2H), 7.52 (s, 1H), 7.25 (d, J=8.5 Hz, 2H), 7.02-6.91 (m, 2H), 6.60-6.47 (m, 3H), 6.36-6.23 (m, 2H), 4.30 (d, J=6.0 Hz, 2H), 3.76 (s, 3H), 3.62 (d, J=12.1 Hz, 4H), 2.59 (d, J=11.8 Hz, 1H), 2.47 (s, 2H), 2.31 (s, 4H), 2.31-2.18 (m, 1H), 2.14 (s, 3H), 1.82 (d, J=12.2 Hz, 2H), 1.57-1.40 (m, 2H), 1.22 (s, 2H).

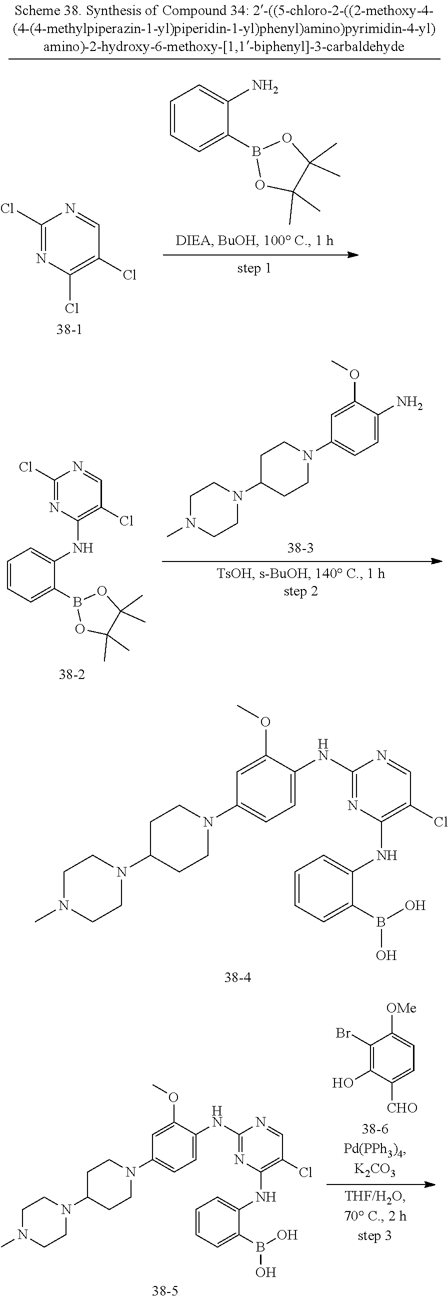

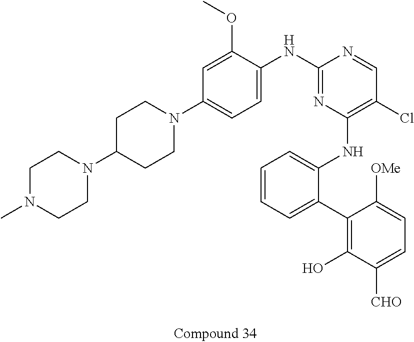 [SMILES: COc1cc(N2CCC(N3CCN(C)CC3)CC2)ccc1Nc1ncc(Cl)c(Nc2ccccc2-c2c(OC)ccc(C=O)c2O)n1]

### Step 1: Synthesis of 2,5-dichloro-N-[2-(4,4,5,5-tetramethyl-1,3,2-dioxaborolan-2-yl)phenyl]pyrimidin-4-amine

2,4,5-trichloropyrimidine (1 g, 5.452 mmol, 1 equiv), 2-(4,4,5,5-tetramethyl-1,3,2-dioxaborolan-2-yl) aniline (1.2 g, 5.477 mmol, 1.00 equiv), DIEA (2 mL, 11.482 mmol, 2.11 equiv), BuOH (10 mL, 109.399 mmol, 20.07 equiv) were added to a 100 mL sized reaction vessel. The reaction mixture was stirred at 100° C. for 2 hours under N₂. The reaction was quenched with water (50 mL). The resulting mixture was extracted with ethyl acetate (3×50 mL). The organic layers were combined, dried over anhydrous sodium sulfate, filtered and concentrated, The residue was purified by silica gel column chromatography, eluted with EA/PE (0-60%) to afford 2,5-dichloro-N-[2-(4,4,5,5-tetramethyl-1,3,2-dioxaborolan-2-yl)phenyl]pyrimidin-4-amine (530 mg, 26.56%) as a yellow solid. MS (ESI) calculated for C₁₆H₁₈BCl₂N₃O_{2-, 365.09} m/z, found: 366.00[M+H]⁺.

### Step 2: 2-{[5-chloro-2-({2-methoxy-4-[4-(4-methylpiperazin-1-yl)piperidin-1-yl]phenyl}amino)pyrimidin-4-yl]amino}phenylboronic acid

2,5-dichloro-N-[2-(4,4,5,5-tetramethyl-1,3,2-dioxaborolan-2-yl)phenyl]pyrimidin-4-amine (250 mg, 0.683 mmol, 1 equiv), 1-[1-(3-methoxyphenyl)piperidin-4-yl]-4-methylpiperazine (197.67 mg, 0.683 mmol, 1 equiv) TsOH (176.41 mg, 1.025 mmol, 1.5 equiv) were added to a 40 mL sized reaction vessel. The reaction mixture was stirred at 140° C. for 1 hours under N₂. To afford 2-{[5-chloro-2-({2-methoxy-4-[4-(4-methylpiperazin-1-yl)piperidin-1-yl]phenyl}amino)pyrimidin-4-yl]amino}phenylboronic acid (200 mg, 53.06%) as a yellow solid. MS (ESI) calculated for C₂₇H₃₅BClN₇O₃, 551.26 m/z, found: 552.20[M+H]⁺.

### Step 3: 2′-{[5-chloro-2-({2-methoxy-4-[4-(4-methylpiperazin-1-yl)piperidin-1-yl]phenyl}amino)pyrimidin-4-yl]amino}-2-hydroxy-6-methoxy-[1,1′-biphenyl]-3-carbaldehyde

2-{[5-chloro-2-({2-methoxy-4-[4-(4-methylpiperazin-1-yl)piperidin-1-yl]phenyl}amino)pyrimidin-4-yl]amino}phenylboronic acid (200 mg, 0.362 mmol, 1 equiv), 3-bromo-2-hydroxy-4-methoxybenzaldehyde (125 mg, 0.541 mmol, 1.49 equiv), Pd(PPh₃)₄(42 mg, 0.036 mmol, 0.10 equiv), K₂CO₃ (101 mg, 0.731 mmol, 2.02 equiv), THF (5 mL, 61.714 mmol, 170.29 equiv), H₂O (1 mL, 55.509 mmol, 153.17 equiv) were added to a 40 mL sized reaction vessel. The reaction was quenched with water (50 mL). The resulting mixture was extracted with ethyl acetate (3×50 mL). The organic layers were combined, dried over anhydrous sodium sulfate, filtered and concentrated, The residue obtained was purified by preparative HPLC using a XSelect CSH Prep C18 OBD Column, 19*250 mm, 5 μm column (eluent: 38% to 35% (v/v) water (0.05% TFA) and MeOH to afford 2′-{[5-chloro-2-({2-methoxy-4-[4-(4-methylpiperazin-1-yl)piperidin-1-yl]phenyl}amino)pyrimidin-4-yl]amino}-2-hydroxy-6-methoxy-[1,1′-biphenyl]-3-carbaldehyde (7.3 mg, 3.02%). Calcd. for C₃₅H₄₀ClN₇O₄, 657.28 m/z, found: 658.30[M+H]⁺. ¹H NMR (300 MHz, DMSO-d₆) δ 11.56 (s, 1H), 9.87 (s, 1H), 8.43 (s, 1H), 8.14 (s, 1H), 8.00 (s, 1H), 7.90-7.81 (m, 2H), 7.47 (s, 1H), 7.40-7.33 (m, 1H), 7.34-7.19 (m, 2H), 6.95-6.87 (m, 1H), 6.72 (s, 1H), 6.52 (d, J=8.8 Hz, 1H), 3.77 (d, J=9.7 Hz, 9H), 3.67-2.91 (m, 8H), 2.80 (s, 5H), 2.04 (d, J=11.9 Hz, 2H), 1.75-1.57 (m, 2H). 19F NMR (282 MHz, DMSO-d₆) δ-74.222.

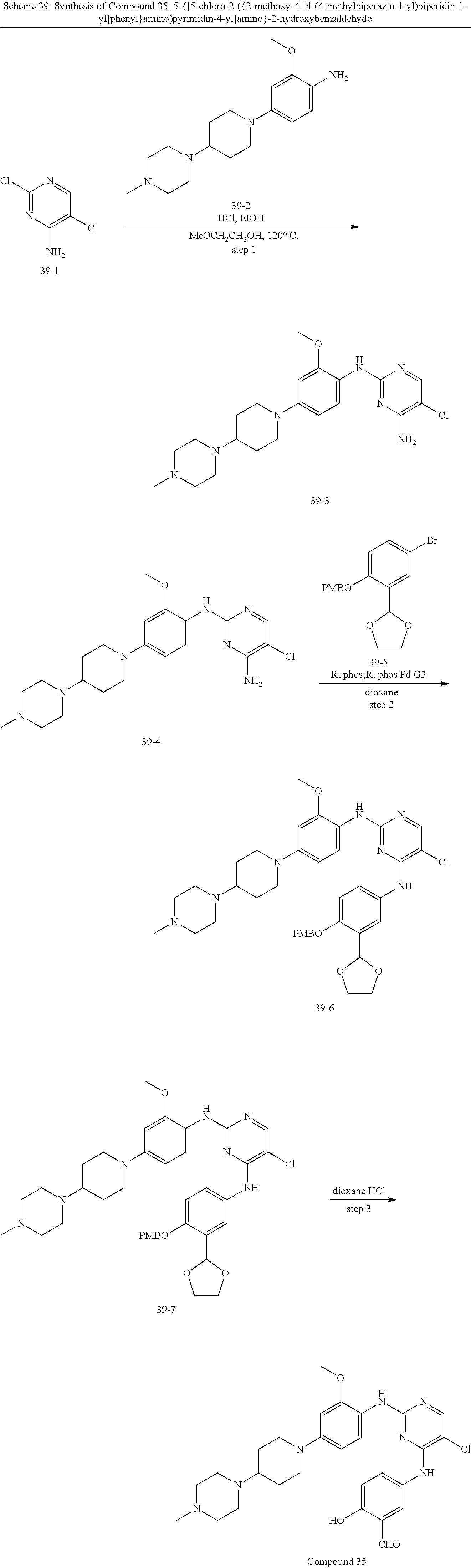

### Step 1: Synthesis of 5-chloro-N2-(2-methoxy-4-(4-(4-methylpiperazin-1-yl)piperidin-1-yl)phenyl)pyrimidine-2,4-diamine

Ethyl 1-(5-hydroxypyridin-3-yl)-1H-pyrazole-4-carboxylate (2.0 g, 8.58 mmol) was added to a mixture of 2-bromo-3-(2,2-difluoroethoxy)pyridine (2.2 g, 9.24 mmol), Cs₂CO₃ (5.5 g, 16.87 mmol), Pd₂(dba)₃, CHCl₃ (0.8 g, 0.85 mmol) and Josiphos SL-J009-1 (0.3 g, 0.85 mmol) in 1,4-dioxane (20 mL). The reaction was stirred at 100° C. for 12 hours under N₂, quenched with water (200 mL) and extracted with EA (200 mL×3). The combined organic extracts were washed with brine, dried over anhydrous Na₂SO₄, filtered and concentrated to dryness under reduced pressure to provide the crude product, which was purified by column chromatography on silica gel with EA/PE (0-80%) to give ethyl 1-(5-((3-(2,2-difluoroethoxy)pyridin-2-yl)oxy)pyridin-3-yl)-1H-pyrazole-4-carboxylate as a brown solid (2.8 g, 83%). MS (ESI): mass calculated for C₂₁H₃₀ClN₇O_{431.22} m/z, found 432.2 [M+H]⁺.

### Step 2: Synthesis of N4-{4-[(tert-butyldimethylsilyl)oxy]-3-(1,3-dioxolan-2-yl)phenyl}-S-chloro-N2-{2-methoxy-4-[4-(4-methylpiperazin-1-yl)piperidin-1-yl]phenyl}pyrimidine-2,4-diamine

5-chloro-N2-{2-methoxy-4-[4-(4-methylpiperazin-1-yl)piperidin-1-yl]phenyl}pyrimidine-2,4-diamine (250 mg, 0.579 mmol, 1 equiv) was added to a mixture of 4-bromo-2-(1,3-dioxolan-2-yl)phenoxy(tert-butyl)dimethylsilane (207.96 mg, 0.579 mmol, 1 equiv), Ruphos (27.01 mg, 0.058 mmol, 0.1 equiv),CS2CO₃ (377.13 mg, 1.158 mmol, 2 equiv) and Ruphos Pd G3 (48.40 mg, 0.058 mmol, 0.1 equiv) in dioxane (5 mL). The reaction was stirred at 100° C. for 12 hours under N₂. The reaction was stirred at 100° C. for 12 hours, quenched with water (10 mL) and extracted with EA (10 mL×3). The combined organic extracts were washed with brine, dried over anhydrous Na₂SO₄, filtered and concentrated to dryness under reduced pressure to provide the crude product, which was purified by column chromatography on silica gel with EA/PE (0-60%) to afford N4-{4-[(tert-butyldimethylsilyl)oxy]-3-(1,3-dioxolan-2-yl)phenyl}-S-chloro-N2-{2-methoxy-4-[4-(4-methylpiperazin-1-yl)piperidin-1-yl]phenyl}pyrimidine-2,4-diamine 300 mg as a brown solid.

MS (ESI): mass calculated for C₃₈H₄₆ClN₇O₅ 715.32 m/z, found 716.3 [M+H]⁺.

### Step 3: Synthesis of 5-{[5-chloro-2-({2-methoxy-4-[4-(4-methylpiperazin-1-yl)piperidin-1-yl]phenyl}amino)pyrimidin-4-yl]amino}-2-hydroxybenzaldehyde

A solution of N4-{4-[(tert-butyldimethylsilyl)oxy]-3-(1,3-dioxolan-2-yl)phenyl}-S-chloro-N2-{2-methoxy-4-[4-(4-methylpiperazin-1-yl)piperidin-1-yl]phenyl}pyrimidine-2,4-diamine (0.05 g, 0.070 mmol, 1 equiv) in dioxane (5 mL) and concentrated HCl (0.5 mL) was stirred for 0.5 h at rt. The resulting mixture was concentrated under vacuum and extracted with EA (50 ml). The combined organic layers were washed with water, dried over anhydrous Na₂SO₄. After filtration, the filtrate was concentrated under reduced pressure. The residue was purified by silica gel column chromatography to afford 5-{[5-chloro-2-({2-methoxy-4-[4-(4-methylpiperazin-1-yl)piperidin-1-yl]phenyl}amino)pyrimidin-4-yl]amino}-2-hydroxybenzaldehyde (3.1 mg, 4.00%) as a yellow semi-solid. MS (ESI) calculated for C₂₈H₃₄ClN₇O₃, 551.24 m/z, found 552.25 [M+H]⁺. ¹H NMR (300 MHz, CD₃OD-d₄) δ (ppm): ¹H NMR (300 MHz, Methanol-d₄) & 8.08-7.90 (m, 1H), 7.53-7.43 (m, 2H), 7.01 (m, 2H), 6.55 (d, J=9.1 Hz, 1H), 5.64 (s, 1H), 3.88 (d, J=3.8 Hz, 3H), 3.81 (d, J=12.3 Hz, 2H), 2.97 (d, J=11.8 Hz, 3H)_{2.84} (s, 3H), 2.21 (m. J=7.5 Hz. 3H). 2.09(d. J=13.2 Hz, 1H), 1.80 (s, 1H), 1.32 (s, 6H), 0.91 (d. J=7.0; H_{2-, 2}H).

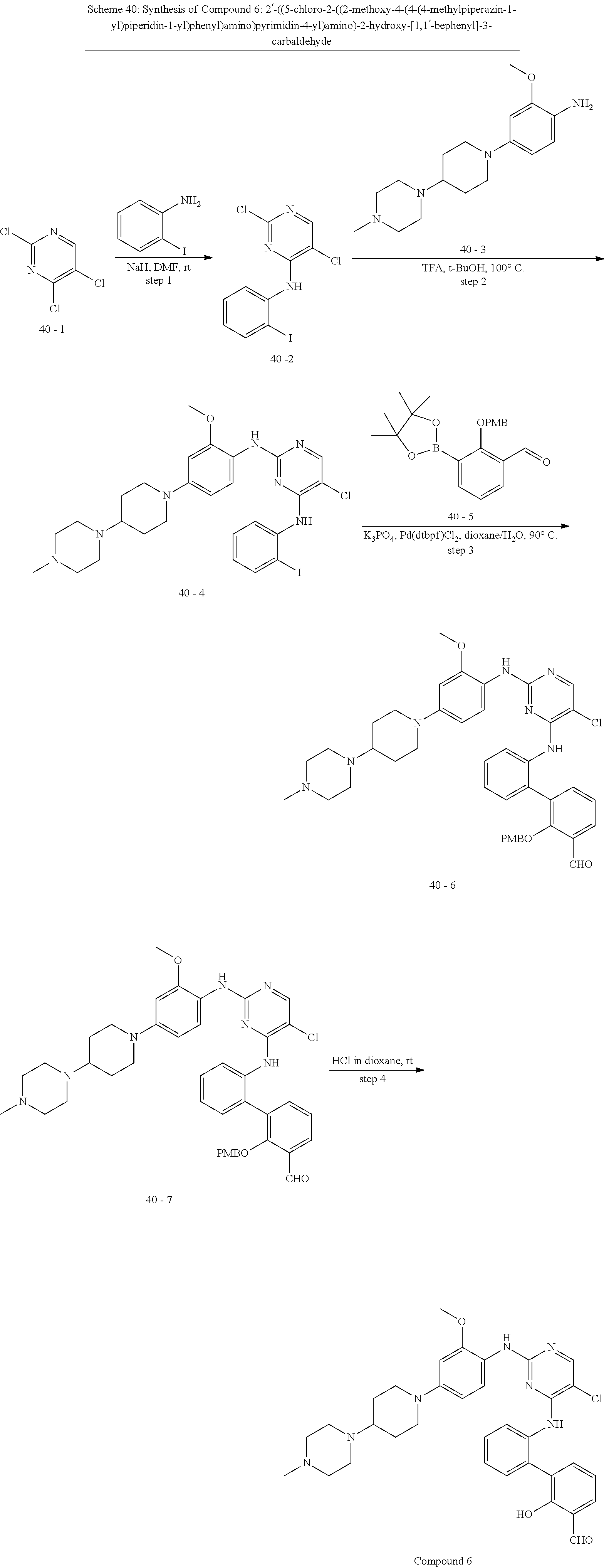

### Step 1: 2,5-dichloro-N-(2-iodophenyl)pyrimidin-4-amine

2,4,5-trichloropyrimidine (2.4 g, 13.085 mmol, 1 equiv), a stir bar, DCM (4 ml) were added to a 100 mL reaction vessel. Then, NaH (0.63 g, 26.170 mmol, 2 equiv) was added to an ice bath and stirred until homogeneous. The reaction mixture was stirred for 0.5h at rt under N₂. Then add 2-iodoaniline (2.87 g, 13.085 mmol, 1 equiv) to reaction mixture was stirred for overnight at rt under N₂. The reaction was filtered and concentrated to dryness to give 2,5-dichloro-N-(2-iodophenyl)pyrimidin-4-amine (2.97 g, 62.02%) as yellow solid. MS (ESI) calculated for C₁₀H₆1N₃, 365.98 m/z, found 366.05[M+H]⁺.

### Step 2: 5-chloro-N4-(2-iodophenyl)-N2-(2-methoxy-4-(4-(4-methylpiperazin-1-yl)piperidin-1-yl)phenyl)pyrimidine-2,4-diamine

2,5-dichloro-N-(2-iodophenyl)pyrimidin-4-amine (500 mg, 1.366 mmol, 1 equiv), a stir bar, 2-methoxy-4-[4-(4-methylpiperazin-1-yl)piperidin-1-yl]aniline (415.92 mg, 1.366 mmol, 1 equiv),TFA (233.67 mg, 2.049 mmol, 1.5 equiv) and t-butanol (2 mL) were added to a 40 mL reaction vessel. The reaction mixture was stirred for 0.5h at 100° C. under N₂. The reaction was filtered and concentrated to dryness to give 5-chloro-N4-(2-iodophenyl)-N2-{2-methoxy-4-[4-(4-methylpiperazin-1-yl)piperidin-1-yl]phenyl}pyrimidine-2,4-diamine (700 mg, 80.82%) as purple solid. MS (ESI) calculated for C₂₇H₃₃Cl₁N₇O_{, 633.96} m/z, found 634.05[M+H]⁺.

### Step 3: 2′-((5-chloro-2-((2-methoxy-4-(4-(4-methylpiperazin-1-yl)piperidin-1-yl)phenyl)amino)pyrimidin-4-yl)amino)-2-((4-methoxybenzyl)oxy)-[1,1′-biphenyl]-3-carbaldehyde

5-chloro-N4-(2-iodophenyl)-N2-{2-methoxy-4-[4-(4-methylpiperazin-1-yl)piperidin-1-yl]phenyl}pyrimidine-2,4-diamine (100 mg, 0.158 mmol, 1 equiv), a stir bar, 2-[(4-methoxyphenyl)methoxy]-3-(4,4,5,5-tetramethyl-1,3,2-dioxaborolan-2-yl)benzaldehyde (116.17 mg, 0.316 mmol, 2 equiv), K₃PO₄ (66.96 mg, 0.316 mmol, 2 equiv),Pd(dTBPf)Cl₂ (10.28 mg, 0.016 mmol, 0.1 equiv) and dioxane (2.5 mL, NaN mmol) were added to a 40 mL reaction vessel. The reaction mixture was stirred for overnight at 80° C. under N₂.The reaction was filtered and concentrated to dryness to give 2′-{[5-chloro-2-({2-methoxy-4-[4-(4-methylpiperazin-1-yl)piperidin-1-yl]phenyl}amino)pyrimidin-4-yl]amino}-2-[(4-methoxyphenyl)methoxy]-[1,1′-biphenyl]-3-carbaldehyde (142 mg, 120.30%) as a light orange solid. MS (ESI) calculated for C₄₂H₄₆ClIN₇O₄, 748.33 m/z, found 747.25[M−H]—.

### Step 4: 2′-((5-chloro-2-((2-methoxy-4-(4-(4-methylpiperazin-1-yl)piperidin-1-yl)phenyl)amino)pyrimidin-4-yl)amino)-2-hydroxy-[1,1′-biphenyl]-3-carbaldehyde

Bis(2′-{[5-chloro-2-({2-methoxy-4-[4-(4-methylpiperazin-1-yl)piperidin-1-yl]phenyl}amino)pyrimidin-4-yl]amino}-2-[(4-methoxyphenyl)methoxy]-[1,1′-biphenyl]-3-carbaldehyde) (100 mg, 0.067 mmol, 1 equiv), a stir bar, dioxane (2 mL), conc. HCl (2 mL) were added to a 20 mL reaction vessel and stirred until homogeneous. The reaction was stirred overnight at rt. After cooling down to rt, the reaction mixture was concentrated to dryness to give brown oil. The brown oil was purified by preparative HPLC using a XSelect CSH Fluoro Phenyl, 30*150 mm, 5 μm column (eluent: 27% to 52% (v/v) ACN and H₂O with 10 mmol/L NH₄HCO₃) to afford bis(2′-{[5-chloro-2-({2-methoxy-4-[4-(4-methylpiperazin-1-yl)piperidin-1-yl]phenyl}amino)pyrimidin-4-yl]amino}-2-hydroxy-[1,1′-biphenyl]-3-carbaldehyde) (8 mg, 9.53%) as a yellow solid. LC/MS: mass calculated for C₃₄H₃₈ClIN₇O₃:627.27, found: 628.25[M+H]⁺. ¹H NMR (300 MHz, DMSO-d6) δ 10.19 (s, 1H), 9.14-9.08 (m, 1H), 7.92 (s, 1H), 7.83 (d, J=8.0 Hz, 1H), 7.75-7.65 (m, 1H), 7.59 (d, J=8.0 Hz, 2H), 7.48-7.31 (m, 3H), 7.28-7.15 (m, 1H), 6.86 (t, J=7.5 Hz, 1H), 6.62-6.55 (m, 1H), 6.42-6.29 (m, 1H), 3.78 (s, 3H), 3.66 (d, J=12.0 Hz, 2H), 2.77-2.58 (m, 6H), 2.55-2.44 (m, 3H), 2.44-2.29 (m, 2H), 2.26 (s, 3H), 1.85 (d, J=12.1 Hz, 2H), 1.69-1.33 (m, 2H).

### Example 3: Representative Compound Syntheses Following General Scheme B

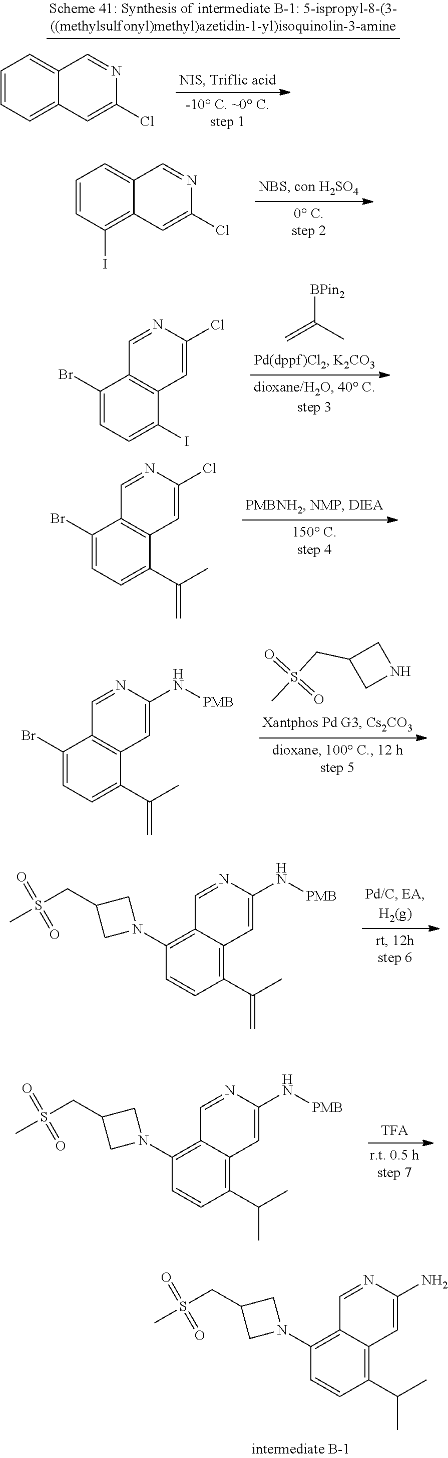

### Step 1: Synthesis of 3-chloro-S-iodoisoquinoline

To a solution of 3-chloroisoquinoline (30 g, 184.1 mmol) in TFA (180 mL) was added NIS (49.6 g, 220.4 mmol). The resulting mixture was stirred at 0° C. for 4 h. The reaction was concentrated. The residue obtained was purified by silica gel chromatography (0-20% EtOAc/petroleum ether) to afford 3-chloro-S-iodoisoquinoline as a yellow solid (20 g, 37.7% yield). MS (ESI) calcd. for C₉H₅Cl₁N, 288.92 m/z, found 289.95 [M+H]⁺.

### Step 2: Synthesis of 8-bromo-3-chloro-S-(prop-1-en-2-yl)isoquinoline

To a solution of 3-chloro-S-iodoisoquinoline (20 g, 69.2 mmol) in H₂SO₄ (400 mL) was added NBS (12.4 g, 69.2 mmol). The resulting mixture was stirred at rt for 5 h. The reaction was concentrated. The residue obtained was purified by silica gel chromatography (0-20% EtOAc/petroleum ether) to afford 2,5-dichloro-N-phenylpyrimidin-4-amine as a yellow solid (15 g, 95.50% yield). MS (ESI) calcd. for C₉H₄BrCl₁N, 366.83 m/z, found 367.95 [M+H]⁺.

### Step 3: Synthesis of 8-bromo-3-chloro-S-iodoisoquinoline

To a 500 ml round-bottomed flask equipped with a stirring bar were added 8-bromo-3-chloro-S-iodoisoquinoline (15 g, 40.9 mmol) and 150 mL dioxane, followed by addition of 4,4,5,5-tetramethyl-2-(prop-1-en-2-yl)-1,3,2-dioxaborolane (8.2 g, 49.1 mmo), Pd(dppf)Cl₂ (3.3 g, 4.1 mmol), K₂CO₃ (0.7 g, 7.119 mmol). The resulting solution was stirred at 40° C. for 2 h under the N2. The resulting mixture was extracted with EA (3 * 10 mL), dried over anhydrous Na₂SO₄. After filtration, the filtrate was concentrated under reduced pressure. The residue obtained was purified by reverse phase column (55% ACN) to afford the 8-bromo-3-chloro-S-(prop-1-en-2-yl)isoquinoline a yellow solid (10g, 87.7% yield). MS (ESI) calcd. for C₁₂H₉BrClN, 280.96 m/z, found 281.95 [M+H]⁺.

### Step 4: Synthesis of 8-bromo-3-chloro-S-(prop-1-en-2-yl)isoquinoline

To a 250 ml round-bottomed flask equipped with a stirring bar were added 8-bromo-3-chloro-S-(prop-1-en-2-yl)isoquinoline (12 g, 42.467 mmol) and 150 mL NMP, followed by addition of PMBNH₂ (11.65 g, 84.934 mmol), DIEA (27.44 g, 212.335 mmol). The resulting solution was stirred at 150° C. for 48 h. The reaction progress was monitored by LCMS, and it showed the reaction was completed. The resulting mixture was extracted with EA (3 * 100 mL), The combined organic layers were washed with water (3 * 20 mL), dried over anhydrous Na₂SO₄. After filtration, the filtrate was concentrated under reduced pressure. The residue was concentrated to dryness in vacuo to give a yellow oil. The yellow oil was purified by silica gel chromatography (9% EA/PE) to afford the 8-bromo-N-(4-methoxybenzyl)-S-(prop-1-en-2-yl)isoquinolin-3-amine as a yellow solid (8 g, 46.52% yield). MS (ESI) calcd. for C₂₀H₁₉BrN₂O_{, 382.07} m/z, found 383.10 [M+H]⁺.

### Step 5: Synthesis of N-(4-methoxybenzyl)-8-(3-((methylsulfonyl)methyl)azetidin-1-yl)-S-(prop-1-en-2-yl)isoquinolin-3-amine

To a 50 ml round-bottomed flask equipped with a stirring bar were added 8-bromo-N-(4-methoxybenzyl)-S-(prop-1-en-2-yl)isoquinolin-3-amine (2.2 g, 1.936 mmol) and 30 mL dioxane, followed by addition of 3-((methylsulfonyl)methyl)azetidine (1.3 g, 8.713 mmo), Xantphos Pd G3 (0.6 g, 0.633 mmo), Cs₂CO₃ (3.7 g, 11.321 mmol). The resulting solution was stirred at 100° C. for 2 h under the N². The resulting mixture was extracted with EA (3 * 30 mL), dried over anhydrous Na₂SO₄. After filtration, the filtrate was concentrated under reduced pressure. The residue was concentrated to dryness in vacuo to give a yellow oil. The yellow oil was purified by silica gel chromatography (70% EA/PE) to afford the N-(4-methoxybenzyl)-8-(3-((methylsulfonyl)methyl)azetidin-1-yl)-S-(prop-i-en-2-yl)isoquinolin-3-amine as a yellow solid (1.5 g, 53.24% yield). MS (ESI) calcd. for C₂₅H₂₉N₃O₃S, 451.19 m/z, found 452.30 [M+H]⁺. Step 6: Synthesis of 5-isopropyl-N-(4-methoxybenzyl)-8-(3-((methylsulfonyl)methyl)azetidin-1-yl)isoquinolin-3-amine

To a 250 ml round-bottomed flask equipped with a stirring bar were added N-(4-methoxybenzyl)-8-(3-((methylsulfonyl)methyl)azetidin-1-yl)-S-(prop-1-en-2-yl)isoquinolin-3-amine (4.5 g, 9.965 mmol) and EA (50 mL), followed by addition of Pd/C (2 g, 1.879 mmol). The mixture was stirred at rt for 12 h under a H₂ (g) (3.5 atm) atmosphere. Organic phase is obtained by filteration and concentrated to dryness to give a yellow solid. The yellow solid was recrystallized with PE (5 mL) to obtain 5-isopropyl-N-(4-methoxybenzyl)-8-(3-((methylsulfonyl)methyl)azetidin-1-yl)isoquinolin-3-amine a yellow solid (2 g, 37.61% yield).

MS (ESI) calcd. for C₂₅H₃₁N₃O₃S, 453.21 m/z, found 454.16 [M+H]⁺.

### Step 7: Synthesis of 5-isopropyl-8-(3-((methylsulfonyl)methyl)azetidin-1-yl)isoquinolin-3-amine

5-isopropyl-N-(4-methoxybenzyl)-8-(3-((methylsulfonyl)methyl)azetidin-1-yl)isoquinolin-3-amine (2 g, 4.409 mmol), a stir bar, trifluoroacetic acid (20 mL) were added to a 50-mL round-bottom flask and stirred until homogeneous. The resulting mixture was stirred at rt for 30 min. The residue was concentrated to dryness in vacuo to give a nearly yellow viscous oil. The crude product was then purified by reverse phase column (30% ACN) to afford the 5-isopropyl-8-(3-((methylsulfonyl)methyl)azetidin-1-yl)isoquinolin-3-amine a yellow solid (900 mg, 58.98% yield). MS (ESI) calcd. for C₁₇H₂₃N₃O₂S, 333.15 m/z, found 334.20 [M+H]⁺.

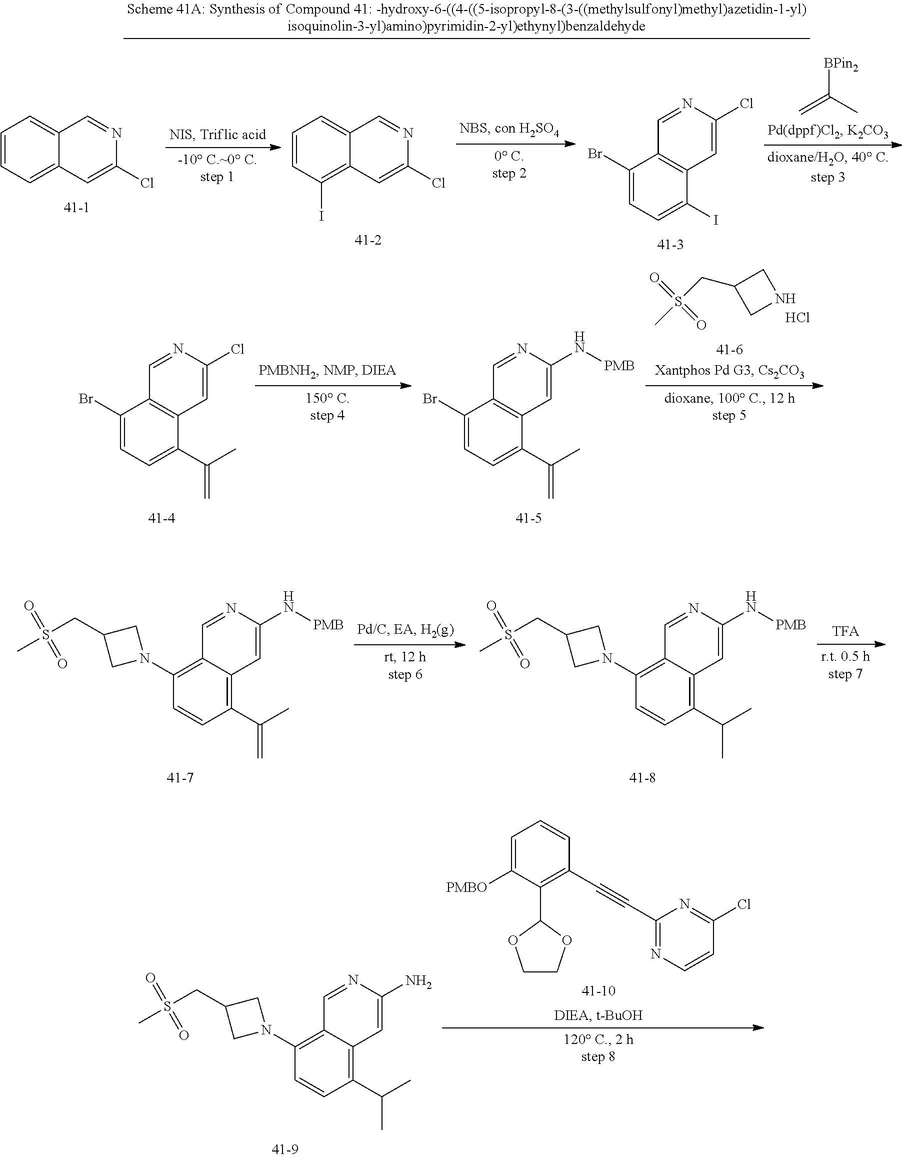

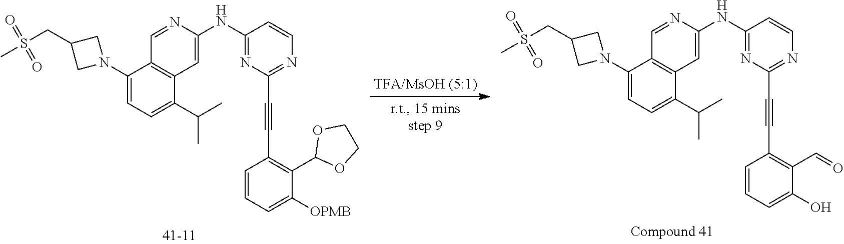

### Step 1: Synthesis of 3-chloro-S-iodoisoquinoline

To a solution of 3-chloroisoquinoline (30 g, 184.1 mmol) in TFA (180 mL) was added NIS (49.6 g, 220.4 mmol). The resulting mixture was stirred at 0° C. for 4 h. The reaction was concentrated. The residue obtained was purified by silica gel chromatography (0-20% EtOAc/petroleum ether) to afford 3-chloro-S-iodoisoquinoline as a yellow solid (20 g, 37.7% yield). MS (ESI) calcd. for C₉H₅Cl₁N, 288.92 m/z, found 289.95 [M+H]⁺.

### Step 2: Synthesis of 8-bromo-3-chloro-S-(prop-1-en-2-yl)isoquinoline

To a solution of 3-chloro-S-iodoisoquinoline (20 g, 69.2 mmol) in H₂SO₄ (400 mL) was added NBS (12.4 g, 69.2 mmol). The resulting mixture was stirred at rt for 5 h. The reaction was concentrated. The residue obtained was purified by silica gel chromatography (0-20% EtOAc/petroleum ether) to afford 2,5-dichloro-N-phenylpyrimidin-4-amine as a yellow solid (15 g, 95.50% yield). MS (ESI) calcd. for C₉H₄BrClIN, 366.83 m/z, found 367.95 [M+H]⁺.

### Step 3: Synthesis of 8-bromo-3-chloro-S-iodoisoquinoline

To a 500 ml round-bottomed flask equipped with a stirring bar were added 8-bromo-3-chloro-S-iodoisoquinoline (15 g, 40.9 mmol) and 150 mL dioxane, followed by addition of 4,4,5,5-tetramethyl-2-(prop-1-en-2-yl)-1,3,2-dioxaborolane (8.2 g, 49.1 mmo), Pd(dppf)Cl₂ (3.3 g, 4.1 mmol), K₂CO₃ (0.7 g, 7.119 mmol). The resulting solution was stirred at 40° C. for 2 h under the N2. The resulting mixture was extracted with EA (3 * 10 mL), dried over anhydrous Na₂SO₄. After filtration, the filtrate was concentrated under reduced pressure. The residue obtained was purified by reverse phase column (55% ACN) to afford the 8-bromo-3-chloro-S-(prop-1-en-2-yl)isoquinoline a yellow solid (10 g, 87.7% yield). MS (ESI) calcd. for C₁₂H₉BrClN, 280.96 m/z, found 281.95 [M+H]⁺.

### Step 4: Synthesis of 8-bromo-3-chloro-S-(prop-1-en-2-yl)isoquinoline

To a 250 ml round-bottomed flask equipped with a stirring bar were added 8-bromo-3-chloro-S-(prop-1-en-2-yl)isoquinoline (12 g, 42.467 mmol) and 150 mL NMP, followed by addition of PMBNH₂ (11.65 g, 84.934 mmol), DIEA (27.44 g, 212.335 mmol). The resulting solution was stirred at 150° C. for 48 h. The reaction progress was monitored by LCMS, and it showed the reaction was completed. The resulting mixture was extracted with EA (3 * 100 mL).

The combined organic layers were washed with water (3 * 20 mL), dried over anhydrous Na₂SO₄. After filtration, the filtrate was concentrated under reduced pressure. The residue was concentrated to dryness in vacuo to give a yellow oil. The yellow oil was purified by silica gel chromatography (9% EA/PE) to afford the 8-bromo-N-(4-methoxybenzyl)-S-(prop-1-en-2-yl)isoquinolin-3-amine as a yellow solid (8 g, 46.52% yield). MS (ESI) calcd. for C₂₀H₁₉BrN₂O, 382.07 m/z, found 383.10 [M+H]⁺.

### Step 5: Synthesis of N-(4-methoxybenzyl)-8-(3-((methylsulfonyl)methyl)azetidin-1-yl)-S-(prop-1-en-2-yl)isoquinolin-3-amine

To a 50 ml round-bottomed flask equipped with a stirring bar were added 8-bromo-N-(4-methoxybenzyl)-S-(prop-1-en-2-yl)isoquinolin-3-amine (2.2 g, 1.936 mmol) and 30 mL dioxane, followed by addition of 3-((methylsulfonyl)methyl)azetidine (1.3 g, 8.713 mmo), Xantphos Pd G3 (0.6 g, 0.633 mmo), Cs₂CO₃ (3.7 g, 11.321 mmol). The resulting solution was stirred at 100° C. for 2 h under the N². The resulting mixture was extracted with EA (3 * 30 mL), dried over anhydrous Na₂SO₄. After filtration, the filtrate was concentrated under reduced pressure. The residue was concentrated to dryness in vacuo to give a yellow oil. The yellow oil was purified by silica gel chromatography (70% EA/PE) to afford the N-(4-methoxybenzyl)-8-(3-((methylsulfonyl)methyl)azetidin-1-yl)-S-(prop-i-en-2-yl)isoquinolin-3-amine as a yellow solid (1.5 g, 53.24% yield). MS (ESI) calcd. for C₂₅H₂₉N₃O₃S, 451.19 m/z, found 452.30 [M+H]⁺.

### Step 6: Synthesis of 5-isopropyl-N-(4-methoxybenzyl)-8-(3-((methylsulfonyl)methyl)azetidin-1-yl)isoquinolin-3-amine

To a 250 ml round-bottomed flask equipped with a stirring bar were added N-(4-methoxybenzyl)-8-(3-((methylsulfonyl)methyl)azetidin-1-yl)-S-(prop-1-en-2-yl)isoquinolin-3-amine (4.5 g, 9.965 mmol) and EA (50 mL), followed by addition of Pd/C (2 g, 1.879 mmol). The mixture was stirred at rt for 12 h under a H₂ (g) (3.5 atm) atmosphere. Organic phase is obtained by filteration and concentrated to dryness to give a yellow solid. The yellow solid was recrystallized with PE (5 mL) to obtain 5-isopropyl-N-(4-methoxybenzyl)-8-(3-((methylsulfonyl)methyl)azetidin-1-yl)isoquinolin-3-amine a yellow solid (2 g, 37.61% yield).

MS (ESI) calcd. for C₂₅H₃₁N₃O₃S, 453.21 m/z, found 454.16 [M+H]⁺.

### Step 7: Synthesis of 5-isopropyl-8-(3-((methylsulfonyl)methyl)azetidin-1-yl)isoquinolin-3-amine

5-isopropyl-N-(4-methoxybenzyl)-8-(3-((methylsulfonyl)methyl)azetidin-1-yl)isoquinolin-3-amine (2 g, 4.409 mmol), a stir bar, trifluoroacetic acid (20 mL) were added to a 50-mL round-bottom flask and stirred until homogeneous. The resulting mixture was stirred at rt for 30 min. The residue was concentrated to dryness in vacuo to give a nearly yellow viscous oil. The crude product was then purified by reverse phase column (30% ACN) to afford the 5-isopropyl-8-(3-((methylsulfonyl)methyl)azetidin-1-yl)isoquinolin-3-amine a yellow solid (900 mg, 58.98% yield). MS (ESI) calcd. for C₁₇H₂₃N₃O₂S, 333.15 m/z, found 334.20 [M+H]⁺.

### Step 8: N-(2-((2-(1,3-dioxolan-2-yl)-3-((4-methoxybenzyl)oxy)phenyl)ethynyl)pyrimidin-4-yl)-S-isopropyl-8-(3-((methylsulfonyl)methyl)azetidin-1-yl)isoquinolin-3-amine

To a 40 mL microwave tube equipped with a stirring bar were added 2-((2-(1,3-dioxolan-2-yl)-3-((4-methoxybenzyl)oxy)phenyl)ethynyl)-4-chloropyrimidine (300 mg, 0.71 mmol), 5-isopropyl-8-(3-((methylsulfonyl)methyl)azetidin-1-yl)isoquinolin-3-amine (283.1 mg, 0.852 mmol) and 15 mL ButOH, followed by addition of DIEA (274.7 mg, 2.13 mmol). The resulting solution was stirred at 120° C. for 2 h. The reaction progress was monitored by LCMS, and it showed the reaction was completed. The resulting mixture was extracted with EA (2×100 mL), dried over anhydrous Na₂SO₄. After filtration, the filtrate was concentrated under reduced pressure. The residue was concentrated to dryness in vacuo to give a white crude. The crude was purified by reverse phase column eluted with CH₃CN and water (0.05% TFA) to afford the N-(2-((2-(1,3-dioxolan-2-yl)-3-((4-methoxybenzyl)oxy)phenyl)ethynyl)pyrimidin-4-yl)-S-isopropyl-8-(3-((methylsulfonyl)methyl)azetidin-1-yl)isoquinolin-3-amine as a yellow solid (170 mg, 33.2% yield). MS (ESI) calcd. for C₄₀H₄₁N₅O₆S, 719.28 m/z, found 720.20[M+H]⁺.

### Step 9: Synthesis of 2-hydroxy-6-((4-((5-isopropyl-8-(3-((methylsulfonyl)methyl)azetidin-1-yl)isoquinolin-3-yl)amino)pyrimidin-2-yl)ethynyl)benzaldehyde

To a 50 ml round-bottomed flask equipped with a stirring bar were added N-(2-((2-(1,3-dioxolan-2-yl)-3-((4-methoxybenzyl)oxy)phenyl)ethynyl)pyrimidin-4-yl)-S-isopropyl-8-(3-((methylsulfonyl)methyl)azetidin-1-yl)isoquinolin-3-amine (170 mg, 0.236 mmol) and 3 mL TFA, the result mixture was stirred at rt for 5 mins under N₂ atmosphere. The mixture was directly purified by reverse phase column eluted with CH₃CN and water (0.05% NH₄HCO₃) to afford the 2-hydroxy-6-((4-((5-isopropyl-8-(3-((methylsulfonyl)methyl)azetidin-1-yl)isoquinolin-3-yl)amino)pyrimidin-2-yl)ethynyl)benzaldehyde as a orange solid (11.7 mg, 8.9% yield). MS (ESI) calcd. for C₃₀H₂₉N₅O₄S, 555.19 m/z, found 556.20[M+H]⁺.¹H-NMR (300 MHz, DMSO-d₆) δ (ppm): 10.51 (s, 1H), 10.50 (s, 1H), 9.10 (s, 1H), 8.65 (s, 1H), 8.42-8.44 (m, 1H), 7.66-7.69 (m, 1H), 7.41-7.43 (m, 2H), 7.27-7.30 (m, 1H), 7.14-7.17 (m, 1H), 6.40-6.43 (m, 1H), 4.40-4.43 (m, 2H), 3.96-3.98 (m, 2H), 3.58-3.60 (m, 2H), 3.20-3.25 (m, 2H), 3.02 (s, 3H), 1.27 (s, 6H).

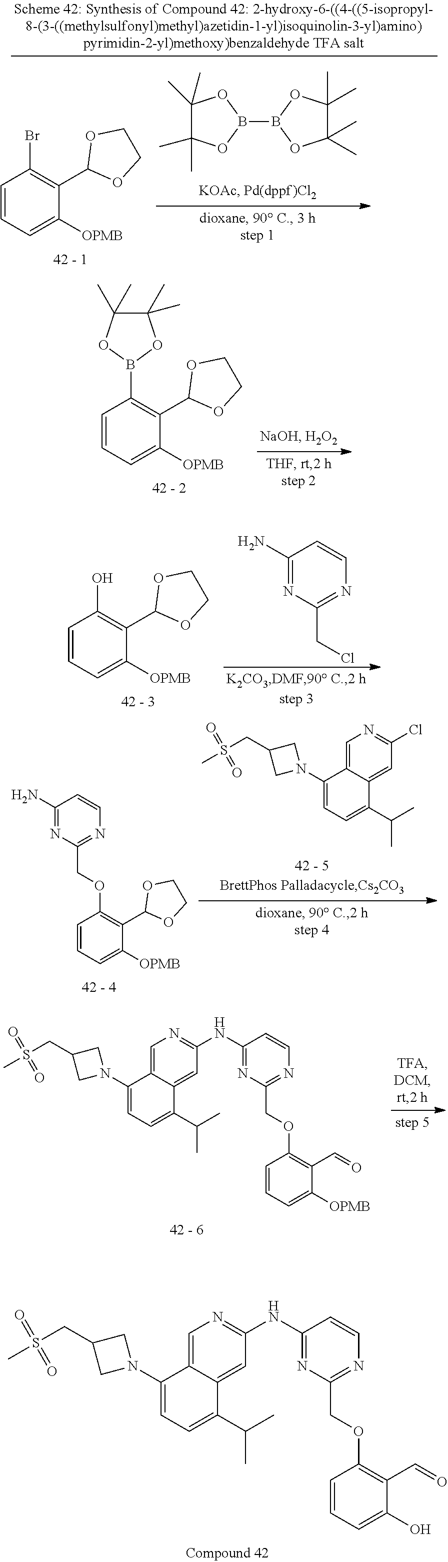

### Step 1: Synthesis of 2-(2-(1,3-dioxolan-2-yl)-3-((4-methoxybenzyl)oxy)phenyl)-4,4,5,5-tetramethyl-1,3,2-dioxaborolane

2-{2-bromo-6-[(4-methoxyphenyl)methoxy]phenyl}-1,3-dioxolane (1 g, 2.738 mmol, 1 equiv), a stir bar, 1,4-dioxane (12 mL) was added to a 100 mL reaction vessel and stirred until homogeneous (˜10 min). Then bis(pinacolato)diboron (1.04 g, 4.107 mmol, 1.5 equiv), Pd(dppf)Cl₂ (0.20 g, 0.274 mmol, 0.1 equiv), KOAc (0.54 g, 5.476 mmol, 2 equiv) was added and the mixture was evacuated and backfilled with nitrogen at rt. The resulting mixture was maintained under nitrogen and stirred at 90° C. for 3 h. The resulting mixture turned into yellow.

After cooling down to rt, the reaction was quenched with water (30 mL). And extracted with ethyl acetate (3×30 mL). The combined organic layer was washed with sat. NaCl (30 ml), dried over anhydrous Na₂SO₄, filtered and concentrated to afford a yellow solid. It was purified by column chromatography on silica gel with (0-36%, PE/EA) to afford 2-[2-(1,3-dioxolan-2-yl)-3-[(4-methoxyphenyl)methoxy]phenyl]-4,4,5,5-tetramethyl-1,3,2-dioxaborolane (700 mg, 62.01%) as a yellow solid. MS (ESI): mass calcd. for C₂₃H₂₉BO₆ 412.21 m/z, found 413.25 [M+H]⁺.

### Step 2: Synthesis of 2-(1,3-dioxolan-2-yl)-3-((4-methoxybenzyl)oxy)phenol

2-[2-(1,3-dioxolan-2-yl)-3-[(4-methoxyphenyl)methoxy]phenyl]-4,4,5,5-tetramethyl-1,3,2-dioxaborolane (690 mg, 1.674 mmol, 1 equiv), a stir bar, THF (9 mL) was added to a 40 mL reaction vessel and stirred until homogeneous (˜10 min). Then NaOH (200.81 mg, 5.022 mmol, 3 equiv), H₂O₂(170.78 mg, 5.022 mmol, 3 equiv) was added and the mixture was evacuated and backfilled with nitrogen at 0° C. The resulting mixture was maintained under nitrogen and stirred at rt for 2 h. The resulting mixture turned into white. The reaction was quenched with water (50 mL). And extracted with ethyl acetate (3×50 mL). The combined organic layer was washed with sat. NaCl (50 ml), dried over anhydrous Na₂SO₄, filtered and concentrated to afford a white solid. It was purified by column chromatography on silica gel with (0-84%, PE/EA) to afford 2-(1,3-dioxolan-2-yl)-3-[(4-methoxyphenyl)methoxy]phenol (280 mg, 55.34%) as a white solid. MS (ESI): mass calcd. for C₁₇H₁₈O₅ 302.12 m/z, found 303.15 [M+H]⁺.

### Step 3: Synthesis of 2-((2-(1,3-dioxolan-2-yl)-3-((4-methoxybenzyl)oxy)phenoxy)methyl) pyrimidin-4-amine

2-(1,3-dioxolan-2-yl)-3-[(4-methoxyphenyl)methoxy]phenol (270 mg, 0.893 mmol, 1 equiv), a stir bar, DMF (5 mL) was added to a 40 mL reaction vessel and stirred until homogeneous (˜10 min). Then 2-(chloromethyl)pyrimidin-4-amine (153.86 mg, 1.072 mmol, 1.2 equiv), K₂CO₃ (246.86 mg, 1.786 mmol, 2 equiv) was added and the mixture was evacuated and backfilled with nitrogen at rt. The resulting mixture was maintained under nitrogen and stirred at 90° C. for 2 h. The resulting mixture turned into yellow. After cooling down to r.t, the reaction was quenched with water (30 mL). And extracted with ethyl acetate (3×30 mL). The combined organic layer was washed with sat. NaCl (30 ml), dried over anhydrous Na₂SO₄, filtered and concentrated to afford a yellow solid. It was purified by column chromatography on silica gel with (0-56%, PE/EA) to afford 2-[2-(1,3-dioxolan-2-yl)-3-[(4-methoxyphenyl)methoxy]phenoxymethyl]pyrimidin-4-amine (220 mg, 60.16%) as a yellow solid. MS (ESI): mass calcd. for C₂₂H₂₃N₃O₅ 409.16 m/z, found 410.20 [M+H]⁺.

### Step 4: Synthesis of 2-((4-((5-isopropyl-8-(3-((methylsulfonyl)methyl)azetidin-1-yl)isoquinolin-3-yl)amino)pyrimidin-2-yl)methoxy)-6-((4-methoxybenzyl)oxy)benzaldehyde

2-[2-(1,3-dioxolan-2-yl)-3-[(4-methoxyphenyl)methoxy]phenoxymethyl]pyrimidin-4-amine (100 mg, 0.244 mmol, 1 equiv), a stir bar, 1,4-dioxane (4 mL) was added to a 40 mL reaction vessel and stirred until homogeneous (˜10 min). Then 3-chloro-S-isopropyl-8-[3-(methanesulfonylmethyl)azetidin-1-yl]isoquinoline (129.28 mg, 0.366 mmol, 1.5 equiv), BrettPhos Palladacycle (19.51 mg, 0.024 mmol, 0.1 equiv), Cs₂CO₃ (159.15 mg, 0.488 mmol, 2 equiv) was added and the mixture was evacuated and backfilled with nitrogen at rt. The resulting mixture was maintained under nitrogen and stirred at 90° C. for 2 h. The resulting mixture turned into yellow. After cooling down to r.t, the reaction was quenched with water (30 mL). And extracted with ethyl acetate (3×30 mL). The combined organic layer was washed with sat. NaCl (30 ml), dried over anhydrous Na₂SO₄, filtered and concentrated to afford a yellow solid. It was purified by reverse C18 column gel with (0-34%, ACN/H₂O) to afford 2-((4-((5-isopropyl-8-(3-((methylsulfonyl)methyl)azetidin-1-yl)isoquinolin-3-yl)amino)pyrimidin-2-yl)methoxy)-6-((4-methoxybenzyl)oxy)benzaldehyde (80 mg, 45.13%) as a yellow solid. MS (ESI): mass calcd. for C₃₇H₃₉N₅O₆S 681.26 m/z, found 682.20 [M+H]⁺.

### Step 5: Synthesis of 2-hydroxy-6-((4-((5-isopropyl-8-(3-((methylsulfonyl)methyl)azetidin-1-yl)isoquinolin-3-yl)amino)pyrimidin-2-yl)methoxy)benzaldehyde TFA salt

2-((4-((5-isopropyl-8-(3-((methylsulfonyl)methyl)azetidin-1-yl)isoquinolin-3-yl)amino)pyrimidin-2-yl)methoxy)-6-((4-methoxybenzyl)oxy)benzaldehyde (70 mg, 0.103 mmol), a stir bar, DCM (4 mL) was added to a 40 mL reaction vessel and stirred until homogeneous (˜10 min). Then TFA (1.17 mg, 0.010 mmol) was added, and the mixture was evacuated and backfilled with nitrogen at rt. The resulting mixture was maintained under nitrogen and stirred at rt for 2 h. The resulting mixture turned into red. The reaction mixture was concentrated to dryness to give red solid. The yellow solid was purified by preparative HPLC using a Xselect CSH C18 OBD, 150 mm×30 mm×5 um column (eluent: 29% o to 46% o (v/v) MeOH and H₂O (0.05% oTFA)) to afford 2-hydroxy-6-((4-((5-isopropyl-8-(3-((methylsulfonyl)methyl)azetidin-1-yl)isoquinolin-3-yl)amino)pyrimidin-2-yl)methoxy)benzaldehyde TFA salt as a red solid (7.4 mg, 10.42% o). MS (ESI): mass calcd. for C₂₉H₃₁NsOSS 561.20 m/z, found 562.20 [M+H]*. ¹H NMR (300 MIHz, DMSO-d6) δ 11.74 (s, 1H), 10.79 (s, 1H), 10.42 (s, 1H), 9.12 (s, 1H), 8.65 (s, 1H), 8.45 (d, J=6.3 Hz, 1H), 7.55 (t, J=8.4 Hz, 1H), 7.48 (s, 1H), 7.45-7.32 (m, 1H), 6.71 (d, J=8.2 Hz, 1H), 6.59 (d, J=8.4 Hz, 1H), 6.44 (d, J=8.1 Hz, 1H), 5.34 (d, J=7.1 Hz, 2H), 4.41 (t, J=7.7 Hz, 2H), 3.99 (t, J=6.9 Hz, 2H), 3.60 (d, J=7.4 Hz, 2H), 3.44-3.22 (m, 2H), 3.02 (s, 3H), 1.22 (d, J=6.8 Hz, 6H). ¹⁹F NMR (282 MHz, DMSO-d6) δ-74.57.

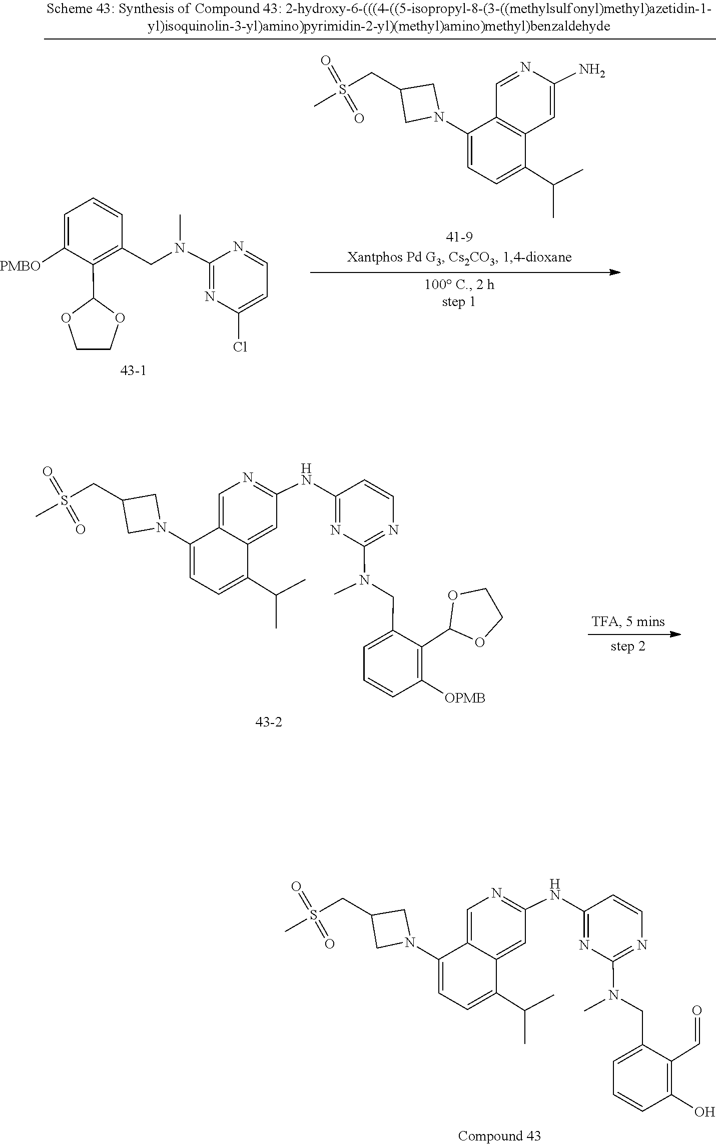

### Step 1: N2-(2-(1,3-dioxolan-2-yl)-3-((4-methoxybenzyl)oxy)benzyl)-N4-(5-isopropyl-8-(3-((methylsulfonyl)methyl)azetidin-1-yl)isoquinolin-3-yl)-N2-methylpyrimidine-2,4-diamine

To a 40 mL microwave tube equipped with a stirring bar were added N-(2-(1,3-dioxolan-2-yl)-3-((4-methoxybenzyl)oxy)benzyl)-4-chloro-N-methylpyrimidin-2-amine (100 mg, 0.226 mmol), 5-isopropyl-8-(3-((methylsulfonyl)methyl)azetidin-1-yl)isoquinolin-3-amine (90.20 mg, 0.271 mmol), Cs₂CO₃ (176.7 mg, 0.542 mol) and 5 mL 1,4-dioxane, followed by addition of XantPhos-Pd G3 (22.0 mg, 0.023 mmol). The resulting solution was stirred at 90° C. for 2 h. The reaction progress was monitored by LCMS, and it showed the reaction was completed. The resulting mixture was extracted with EA (2×100 mL), dried over anhydrous Na₂SO₄. After filtration, the filtrate was concentrated under reduced pressure. The residue was concentrated to dryness in vacuo to give a yellow crude product. The crude product was purified by reverse phase column eluted with CH₃CN and water (0.05% TFA) to afford the N2-(2-(1,3-dioxolan-2-yl)-3-((4-methoxybenzyl)oxy)benzyl)-N4-(5-isopropyl-8-(3-((methylsulfonyl)methyl)azetidin-1-yl)isoquinolin-3-yl)-N2-methylpyrimidine-2,4-diamine as a yellow solid (65 mg, 44.6% yield). MS (ESI) calcd. for C₄₀H₄₆N₆O₆S, 738.32 m/z, found 739.30[M+H]⁺.

### Step 2: Synthesis of 2-hydroxy-6-(((4-((5-isopropyl-8-(3-((methylsulfonyl)methyl)azetidin-1-yl)isoquinolin-3-yl)amino)pyrimidin-2-yl)(methyl)amino)methyl)benzaldehyde

To a 50 ml round-bottomed flask equipped with a stirring bar were added N2-(2-(1,3-dioxolan-2-yl)-3-((4-methoxybenzyl)oxy)benzyl)-N4-(5-isopropyl-8-(3-((methylsulfonyl)methyl)azetidin-1-yl)isoquinolin-3-yl)-N2-methylpyrimidine-2,4-diamine (65 mg, 0.088 mmol) and 3 mL TFA, the result mixture was stirred at rt for 5 mins under N₂ atmosphere. The mixture was directly purified by reverse phase column eluted with CH₃CN and water (0.1% FA) to afford the -hydroxy-6-(((4-((5-isopropyl-8-(3-((methylsulfonyl)methyl)azetidin-1-yl)isoquinolin-3-yl)amino)pyrimidin-2-yl)(methyl)amino)methyl)benzaldehyde formic acid as a yellow solid (4.6 mg, 9.0% yield). MS (ESI) calcd. for C₃₀H₃₄N₆O₄S, 574.24 m/z, found 575.20[M+H]⁺. ¹H-NMR (300 MHz, DMSO-d⁶) δ (ppm): 10.54 (s, 1H), 9.80 (s, 1H), 9.04 (s, 1H), 8.62 (s, 1H), 7.92-7.98 (m, 1H), 7.48-7.50 (m, 2H), 7.33-7.36 (m, 1H), 6.89-6.92 (m, 1H), 6.70-6.72 (m, 2H), 6.52-6.54 (m, 1H), 6.37-6.39 (m, 1H), 5.25 (s, 2H), 4.40-4.43 (m, 2H), 3.96-3.98 (m, 2H), 3.58-3.60 (m, 2H), 3.30 (s, 3H), 3.20-3.25 (m, 2H), 3.02 (s, 3H), 1.23 (s, 6H).

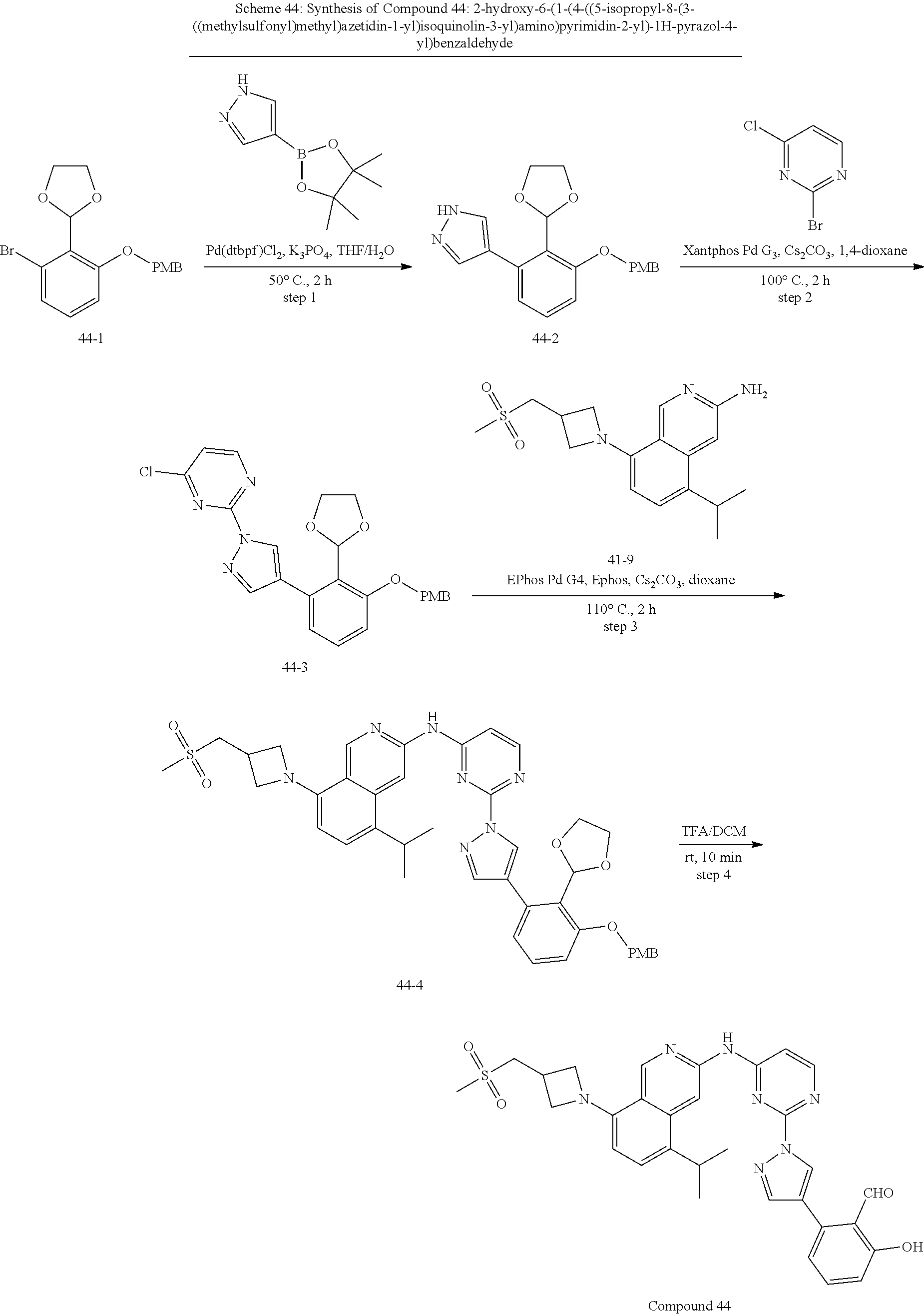

### Step 1: Synthesis of 4-(2-(1,3-dioxolan-2-yl)-3-((4-methoxybenzyl)oxy)phenyl)-1H-pyrazole

To a 25 ml round-bottomed flask equipped with a stirring bar were added (E)-15-bromo-52,53-dihydro-51H-11-oxa-4-aza-5(2,1)-benzo[d]imidazola-2(2,4)-pyridina-1(1,2)-benzenacycloundecaphan-3-one (10 mg, 0.021 mmol) in 1 mL dioxane and 0.3H₂O, followed by addition of 2-(2-(1,3-dioxolan-2-yl)-3-((4-methoxybenzyl)oxy)phenyl)-4,4,5-trimethyl-1,3,2-dioxaborolane (11 mg, 6.184 mmol), Pd(dtbpf)Cl₂ (1.4 mg, 0.002 mmol) and K₃PO₄ (13 mg, 0.061 mmol). The resulting solution was stirred at 50° C. for 2 h under the N2. The resulting mixture was extracted with EA (3 * 10 mL), dried over anhydrous Na₂SO₄. After filtration, the filtrate was concentrated under reduced pressure. MS (ESI) calcd. for C₂₀H₂₀N₂O₄, 352.14 m/z, found 353.15 [M+H]⁺.

### Step 2: Synthesis of 2-(4-(2-(1,3-dioxolan-2-yl)-3-((4-methoxybenzyl)oxy)phenyl)-1H-pyrazol-1-yl)-4-chloropyrimidine

To a 25 ml round-bottomed flask equipped with a stirring bar were added 4-(2-(1,3-dioxolan-2-yl)-3-((4-methoxybenzyl)oxy)phenyl)-1H-pyrazole (20 mg, 0.057 mmol) and 2 mL dioxane, followed by addition of 2-bromo-4-chloropyrimidine (13 mg, 0.067 mmol), Xantphos Pd G3 (5.5 mg, 0.006 mmol), Cs₂CO₃ (37 mg, 0.113 mmol). The resulting solution was stirred at 100° C. for 12 h under the N2. The resulting mixture was extracted with EA (3×30 mL), dried over anhydrous Na₂SO₄. After filtration, the filtrate was concentrated under reduced pressure. The residue was concentrated to dryness in vacuo to give a yellow oil. MS (ESI) calcd. for C₂₄H₂₁ClN₄04, 464.13 m/z, found 465.15 [M+H]⁺.

### Step 3: Synthesis of N-(2-(4-(2-(1,3-dioxolan-2-yl)-3-((4-methoxybenzyl)oxy)phenyl)-1H-pyrazol-1-yl)pyrimidin-4-yl)-S-isopropyl-8-(3-((methylsulfonyl)methyl)azetidin-1-yl)isoquinolin-3-amine

To a 50 ml round-bottomed flask equipped with a stirring bar were added 2-(4-(2-(1,3-dioxolan-2-yl)-3-((4-methoxybenzyl)oxy)phenyl)-1H-pyrazol-1-yl)-4-chloropyrimidine (90 mg, 0.194 mmol) and 2 mL dioxane, followed by addition of 2-(4-(2-(1,3-dioxolan-2-yl)-3-((4-methoxybenzyl)oxy)phenyl)-1H-pyrazol-1-yl)-4-chloropyrimidine (78 mg, 0.234 mmo), EPhos Pd G4 (36 mg, 0.039 mmo), Ephos (30 mg, 0.056 mmol) and caesio methaneperoxoate caesium (140 mg, 0.428 mmol). The resulting solution was stirred at 110° C. for 2 h under the N2. The resulting mixture was extracted with EA (3×10 mL), dried over anhydrous Na₂SO₄. After filtration, the filtrate was concentrated under reduced pressure. The residue obtained was purified by reverse phase column (55% ACN) to afford the 2-hydroxy-6-(1-(4-((5-isopropyl-8-(3-((methylsulfonyl)methyl)azetidin-1-yl)isoquinolin-3-yl)amino)pyrimidin-2-yl)-1H-pyrazol-4-yl)benzaldehyde a white solid (30 mg, 20.34% yield). MS (ESI) calcd. for C₄₁H₄₃N₇O₆S, 597.22 m/z, found 598.40 [M+H]⁺.

### Step 4: Synthesis of 2-hydroxy-6-(1-(4-((5-isopropyl-8-(3-((methylsulfonyl)methyl)azetidin-1-yl)isoquinolin-3-yl)amino)pyrimidin-2-yl)-1H-pyrazol-4-yl)benzaldehyde

N-(2-(4-(2-(1,3-dioxolan-2-yl)-3-((4-methoxybenzyl)oxy)phenyl)-1H-pyrazol-1-yl)pyrimidin-4-yl)-S-isopropyl-8-(3-((methylsulfonyl)methyl)azetidin-1-yl)isoquinolin-3-amine (30 mg, 0.039 mmol, 1 equiv), a stir bar, trifluoroacetic acid (1 mL) and DCM (1 mL) were added to a 25-mL round-bottom flask and stirred until homogeneous. The resulting mixture was stirred at rt for 10 min. The residue was concentrated to dryness in vacuo to give a nearly yellow viscous oil. The crude product was then purified by Prep-HPLC with the following conditions: Column: Xselect CSH C18 OBD Column 30*150 mm 5 μm, n; Mobile Phase A: Water(0.1% FA), Mobile Phase B: ACN; Flow rate: 60 mL/min; Gradient: 42% B to 58% B in 10 min; Wave Length: 254 nm, 598 nm: ; RT1(min): 10.2; Number Of Runs: 3. After lyophilization, it was afforded 2-hydroxy-6-(1-(4-((5-isopropyl-8-(3-((methylsulfonyl)methyl)azetidin-1-yl)isoquinolin-3-yl)amino)pyrimidin-2-yl)-1H-pyrazol-4-yl)benzaldehyde (4.2 mg, 17.33%) as a red solid. MS (ESI) calcd. for C₃₁H₃₁N₇O₄S, 597.22 m/z, found 598.25 [M+H]⁺. ¹H NMR (300 MHz, DMSO-d6) δ (ppm): 11.82 (s, 1H), 10.88 (s, 1H), 10.17 (s, 1H), 9.20 (s, 1H), 9.09 (s, 1H), 8.90 (s, 1H), 8.42-8.49 (m, 1H), 8.10-8.25 (m, 1H), 7.55-7.68 (m, 1H), 7.38-7.48 (m, 1H), 7.18-7.25 (m, 1H), 7.10-7.17 (m, 1H), 6.95-7.05 (m, 1H), 6.41-6.50 (m, 1H), 4.35-4.45 (m, 2H), 3.90-4.03 (m, 2H), 3.75-3.89 (m, 1H), 3.50-3.60 (m, 2H), 3.01 (s, 4H), 3.18-3.45 (m, 6H).

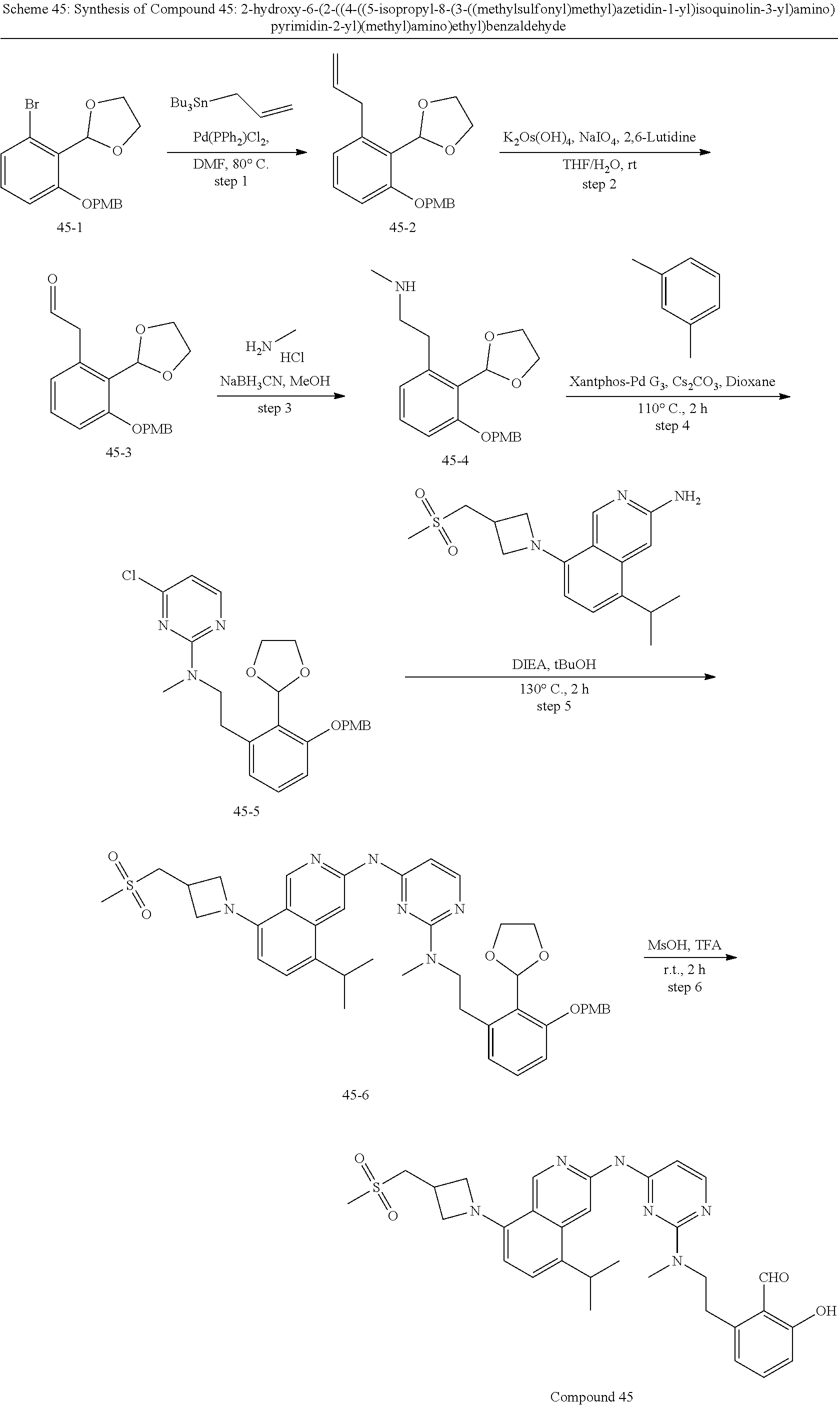

### Step 1: Synthesis of 2-(2-allyl-6-((4-methoxybenzyl)oxy)phenyl)-1,3-dioxolane

To a 100 ml round-bottomed flask equipped with a stirring bar were added methyl 2-{2-bromo-6-[(4-methoxyphenyl)methoxy]phenyl}-1,3-dioxolane (2 g, 5.476 mmol, 1 equiv) and DMF (15 mL, 193.823 mmol, 35.39 equiv), followed by addition of tributyl(prop-2-en-1-yl)stannane (2.72 g, 8.214 mmol, 1.5 equiv), Pd(PPh3)₂C₁₂ (0.77 g, 1.095 mmol, 0.2 equiv). The resulting solution was stirred at 80° C. for 2 h under the N2. The resulting mixture was extracted with EA (3×200 mL), dried over anhydrous Na₂SO₄. After filtration, the filtrate was concentrated under reduced pressure. The residue was purified by silica gel column chromatography, eluted with PE/EA (6%) to afford 2-{2-[(4-methoxyphenyl)methoxy]-6-(prop-2-en-1-yl)phenyl}-1,3-dioxolane (900 mg, 49.59%). MS (ESI) calcd. for C₂₀H₂₂04, 326.15 m/z, found 325.16 [M+H]⁻.

### Step 2: Synthesis of 2-(2-(1,3-dioxolan-2-yl)-3-((4-methoxybenzyl)oxy)phenyl)acetaldehyde

Into a 100 mL flask were added 2-{2-[(4-methoxyphenyl)methoxy]-6-(prop-2-en-1-yl)phenyl}-1,3-dioxolane (600 mg, 1.838 mmol, 1 equiv) and potassium osmate(VI) dihydrate (67.73 mg, 0.184 mmol, 0.1 equiv) and THF (6 mL, 74.057 mmol, 40.29 equiv) 0° C. This was followed by the addition of a solution of NaIO₄ (1572.76 mg, 7.352 mmol, 4 equiv) in H₂O (2 mL, 111.019 mmol, 60.39 equiv), which was added dropwise with stirring at 0° C. degrees c. in 2 mins. To the above mixture was added 2,6-lutidine (393.97 mg, 3.676 mmol, 2 equiv) dropwise/in portions over 30s at 0° C. The resulting mixture was stirred for additional 20 min at 25° C. The desired product was detected by LCMS. The reaction was then quenched by the addition of 50 mL of saturated NH₄Cl. The aqueous layer was extracted with EA (3×50 mL).The residue was purified by silica gel column chromatography, eluted with PE/EA (6% EA) to afford as a white solid.2-[2-(1,3-dioxolan-2-yl)-3-[(4-methoxyphenyl)methoxy]phenyl]acetaldehyde (300 mg, 44.28%). MS (ESI) calcd. for C₁₉H₂₀05, 328.36 m/z, found 329.26 [M+H]⁺.

### Step 3: Synthesis of 2-(2-(1,3-dioxolan-2-yl)-3-((4-methoxybenzyl)oxy)phenyl)acetaldehyde

A solution of Methylamine hydrochloride (493.49 mg, 7.312 mmol, 8 equiv) in MeOH (5 mL, 123.494 mmol, 135.17equiv) was treated with 2-[2-(1,3-dioxolan-2-yl)-3-[(4-methoxyphenyl)methoxy]phenyl]acetaldehyde (300 mg, 0.914 mmol, 1 equiv) for 10 min at 0° C. by the addition of NaBH3CN (401.88 mg, 6.398 mmol, 7 equiv) dropwise/in portions at 0° C. Desired product was detected by LCMS. The reaction was then quenched by the addition of 50 mL of Saturated NH₄Cl. The aqueous layer was extracted with EA (3×50 mL). The residue was purified by silica gel column chromatography, eluted with PE/EA (9% EA) to afford as a white solid. {2-[2-(1,3-dioxolan-2-yl)-3-[(4-methoxyphenyl)methoxy]phenyl]ethyl}(methyl)amine (100 mg, 31.87%) MS (ESI) calcd. for C₂₀H₂₅NO₄, 343.42 m/z, found 344.52 [M+H]⁺.

### Step 4: Synthesis of N-(2-(1,3-dioxolan-2-yl)-3-((4-methoxybenzyl)oxy) phenethyl)-4-chloro-N-methylpyrimidin-2-amine

To a 50 ml round-bottomed flask equipped with a stirring bar were added methyl {2-[2-(1,3-dioxolan-2-yl)-3-[(4-methoxyphenyl)methoxy]phenyl]ethyl}(methyl)amine (140 mg, 0.408 mmol, 1 equiv) and Dioxane (2 mL, 23.608 mmol, 57.91 equiv), followed by addition of 2-bromo-4-chloropyrimidine (86.74 mg, 0.449 mmol, 1.1 equiv). Xantphos Pd G3 (38.66 mg, 0.041 mmol, 0.1 equiv), Cs₂CO₃ (265.65 mg, 0.816 mmol, 2 equiv). The resulting solution was stirred at 80° C. for 2 h under the N2. The resulting mixture was extracted with EA (3×10 mL), dried over anhydrous Na₂SO₄. After filtration, the filtrate was concentrated under reduced pressure. The residue obtained was purified by reverse phase column (55% ACN) to afford the 4-chloro-N-{2-[2-(1,3-dioxolan-2-yl)-3-[(4-methoxyphenyl)methoxy]phenyl]ethyl}-N-methylpyrimidin-2-amine (70 mg, 33.98%). MS (ESI) calcd. for C₂₄H₂₆ClN₃O₄, 455.16 m/z, found 456.16 [M+H]⁺.

### Step 5: Synthesis of N2-(2-(1,3-dioxolan-2-yl)-3-((4-methoxybenzyl)oxy)phenethyl)-N4-(5-isopropyl-8-(3-((methylsulfonyl)methyl)azetidin-1-yl)isoquinolin-3-yl)-N2-methylpyrimidine-2,4-diamine

To a 50 ml round-bottomed flask equipped with a stirring bar were added methyl 4-chloro-N-{2-[2-(1,3-dioxolan-2-yl)-3-[(4-methoxyphenyl)methoxy]phenyl]ethyl}-N-methylpyrimidin-2-amine (70 mg, 0.154 mmol, 1 equiv) and Dioxane (1.5 mL, 17.706 mmol, 115.33 equiv), followed by addition of 5-isopropyl-8-[3-(methanesulfonylmethyl)azetidin-1-yl]isoquinolin-3-amine (56.31 mg, 0.169 mmol, 1.1 equiv), Xantphos Pd G3 (14.56 mg, 0.015 mmol, 0.1 equiv), Cs₂CO₃ (100.05 mg, 0.308 mmol, 2 equiv). The resulting solution was stirred at 80° C. for 2 h under the N2. The mixture was allowed to cool down to 25° C. After filtration, the filtrate was concentrated under reduced pressure. The residue obtained was purified by reverse phase column (25% ACN) to afford the N2-{2-[2-(1,3-dioxolan-2-yl)-3-[(4-methoxyphenyl)methoxy]phenyl]ethyl}-N4-{5-isopropyl-8-[3-(methanesulfonylmethyl)azetidin-1-yl]isoquinolin-3-yl}-N2-methylpyrimidine-2,4-diamine (30 mg, 23.94%). MS (ESI) calcd. for C₄₁H₄₈N₆O₆S, 752.34 m/z, found 753.34 [M+H]⁺.

### Step 6: Synthesis of 2-hydroxy-6-(2-((4-((5-isopropyl-8-(3-((methylsulfonyl)methyl)azetidin-1-yl)isoquinolin-3-yl)amino)pyrimidin-2-yl)(methyl)amino)ethyl)benzaldehyde

To a stirred solution of N2-{2-[2-(1,3-dioxolan-2-yl)-3-[(4-methoxyphenyl)methoxy]phenyl]ethyl}-N4-{5-isopropyl-8-[3-(methanesulfonylmethyl)azetidin-1-yl]isoquinolin-3-yl}-N2-methylpyrimidine-2,4-diamine (30 mg, 0.040 mmol, 1 equiv) in TFA (1.0 mL) and MsOH (0.2 ml) at room temperature. The resulting mixture was stirred for 10 min at room temperature. The resulting mixture was concentrated under reduced pressure. The crude product (45 mg) was purified by Prep-HPLC with the following conditions (Column: XSelect CSH Prep C18 OBD Column, 19*250 mm, 5 μm; Mobile Phase A: Water (0.1% TFA), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 2-hydroxy-6-(2-{[4-({5-isopropyl-8-[3-(methanesulfonylmethyl)azetidin-1-yl]isoquinolin-3-yl}amino)pyrimidin-2-yl](methyl)amino}ethyl)benzaldehyde (2.2 mg, 9.23%) as a yellow solid. MS (ESI) calcd. for C₃₁H₃₆N₆O₄S 588.25 m/z, CMS:589.30[M+H]⁺. ¹H NMR (300 MHz, DMSO-d⁶) δ (ppm): 10.46 (s, 1H), 9.82 (s, 1H), 9.05 (s, 1H), 8.7 (s, 1H), 8.00-8.02 (m, 1H), 7.37-7.39 (m, 2H), 6.80-6.83 (m, 2H), 6.46-6.47 (m, 2H), 6.39-6.41 (m, 2H), 4.39-4.41 (m, 2H), 3.99-4.00 (m, 2H), 3.94-3.96 (m, 2H), 3.85-3.86 (m, 2H), 3.34 (s, 3H), 3.22-3.24 (m, 4H), 3.02 (m, 3H), 1.21-1.23 (m, 6H).

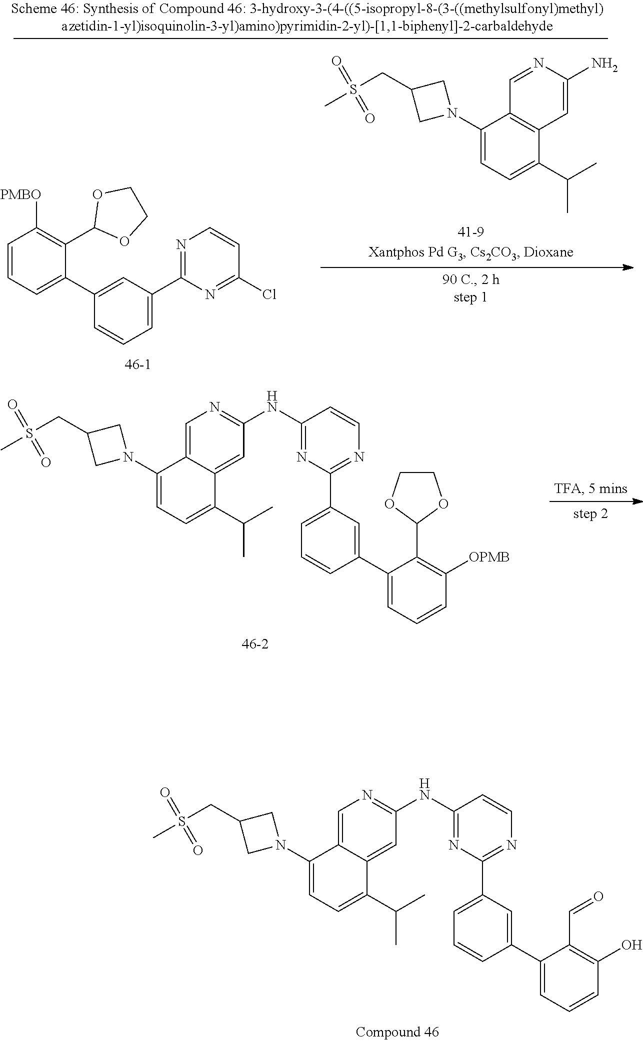

### Step 1: N-(2-(2′-(1,3-dioxolan-2-yl)-3′-((4-methoxybenzyl)oxy)-[1,1′-biphenyl]-3-yl)pyrimidin-4-yl)-S-isopropyl-8-(3-((methylsulfonyl)methyl)azetidin-1-yl)isoquinolin-3-amine

2-(2′-(1,3-dioxolan-2-yl)-3′-((4-methoxybenzyl)oxy)-[1,1′-biphenyl]-3-yl)-4-chloropyrimidine (110 mg, 0.232 mmol), 5-isopropyl-8-(3-((methylsulfonyl)methyl)azetidin-1-yl)isoquinolin-3-amine (178.8 mg, 0.278 mmol), Cs₂CO₃ (151.3 mg, 0.464 mol) and 5 mL 1,4-dioxane, followed by addition of XantPhos-Pd G3 (22.0 mg, 0.023 mmol) was added to a 40 mL microwave tube equipped with a stirring bar. The resulting solution was stirred at 90° C. for 2 h. The reaction progress was monitored by LCMS, and it showed the reaction was completed.

The resulting mixture was extracted with EA (2×100 mL), dried over anhydrous Na₂SO₄. After filtration, the filtrate was concentrated under reduced pressure. The residue was concentrated to dryness in vacuo to give a yellow crude product. The crude product was purified by reverse phase column eluted with CH₃CN and water (0.05% TFA) to afford the N-(2-(2′-(1,3-dioxolan-2-yl)-3′-((4-methoxybenzyl)oxy)-[1,1′-biphenyl]-3-yl)pyrimidin-4-yl)-S-isopropyl-8-(3-((methylsulfonyl)methyl)azetidin-1-yl)isoquinolin-3-amine as a yellow solid (80 mg, 44.6% yield). MS (ESI) calcd. for C₄₄H₄₅N₅O₆S, 771.31 m/z, found 772.30[M+H]⁺.

### Step 2: Synthesis of 2-hydroxy-6-((4-((5-isopropyl-8-(3-((methylsulfonyl)methyl)azetidin-1-yl)isoquinolin-3-yl)amino)pyrimidin-2-yl)ethynyl)benzaldehyde

To a 50 ml round-bottomed flask equipped with a stirring bar were added N-(2-(2′-(1,3-dioxolan-2-yl)-3′-((4-methoxybenzyl)oxy)-[1,1′-biphenyl]-3-yl)pyrimidin-4-yl)-S-isopropyl-8-(3-((methylsulfonyl)methyl)azetidin-1-yl)isoquinolin-3-amine (80 mg, 0.104 mmol) and 3 mL TFA, the result mixture was stirred at rt for 5 mins under N₂ atmosphere. The mixture was directly purified by reverse phase column eluted with CH₃CN and water (0.05% NH₄HCO₃) to afford the 3-hydroxy-3′-(4-((5-isopropyl-8-(3-((methylsulfonyl)methyl)azetidin-1-yl)isoquinolin-3-yl)amino)pyrimidin-2-yl)-[1,1′-biphenyl]-2-carbaldehyde as a red solid (6.3 mg, 8.9% yield). MS (ESI) calcd. for C₃₄H₃₃N₅O₄S, 607.23 m/z, found 608.20[M+H]⁺. ¹H-NMR (300 MHz, DMSO-d₆) δ (ppm): 11.63 (s, 1H), 10.59 (s, 1H), 9.94 (s, 1H), 9.12 (s, 1H), 8.73 (s, 1H), 8.42-8.44 (m, 2H), 8.40-8.50 (m, 1H), 7.41-7.43 (m, 2H), 7.64-7.71 (m, 3H), 7.27-7.34 (m, 2H), 7.01-7.10 (m, 2H), 6.41-6.45 (m, 1H), 4.40-4.43 (m, 2H), 3.96-3.98 (m, 2H), 3.58-3.60 (m, 2H), 3.20-3.25 (m, 2H), 3.02 (s, 3H), 1.27 (s, 6H).

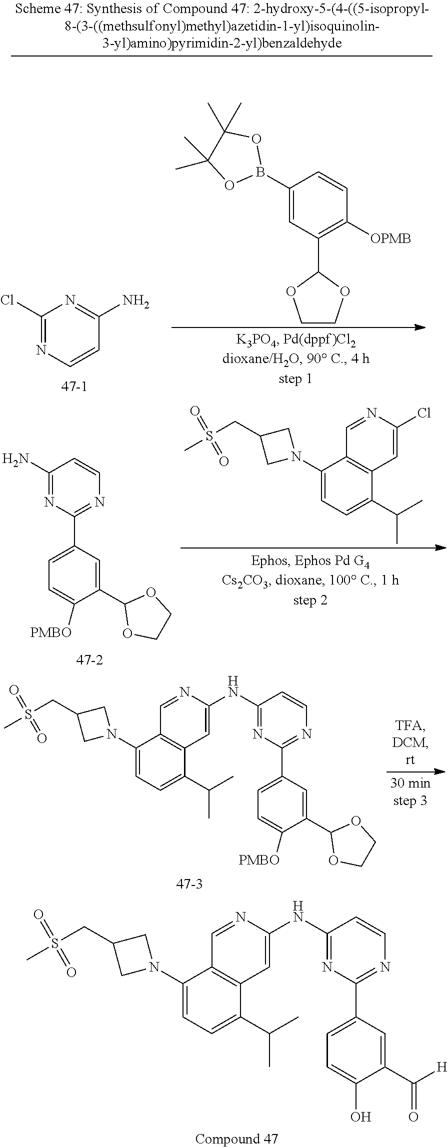

### Step 1: Synthesis of 2-(3-(1,3-dioxolan-2-yl)-4-((4-methoxybenzyl)oxy)phenyl)pyrimidin-4-amine

2-chloropyrimidin-4-amine (100 mg, 0.772 mmol, 1 equiv) and H₂O (0.6 mL, 33.306 mmol), 1,4-dioxane (2.4 mL, 28.330 mmol) were added into 40 ml reaction vessel, a stir bar, then K₃PO₄ (327.70 mg, 1.544 mmol, 2 equiv), Pd(dppf)Cl₂ (56.48 mg, 0.077 mmol, 0.1 equiv) and 2-[3-(1,3-dioxolan-2-yl)-4-[(4-methoxyphenyl)methoxy]phenyl]-4,4,5,5-tetramethyl-1,3,2-dioxaborolane (381.90 mg, 0.926 mmol, 1.2 equiv) were added to a 40 ml reaction vessel and stirred until homogeneous. Nitrogen protection throughout the reaction process. The reaction was stirred overnight at 90° C. Desired product was detected by LCMS. The resulting mixture was extracted with EA100 mL. The combined organic layers were dried over anhydrous sodium sulfate. After filtration, the filtrate was concentrated under reduced pressure. The residue was purified by silica gel column chromatography, eluted with DCM:MEOH(8:1) to afford 2-[3-(1,3-dioxolan-2-yl)-4-[(4-methoxyphenyl)methoxy]phenyl]pyrimidin-4-amine) as a yellow solid. MS (ESI) calcd. for C₂₁H₂₁N₃O₄, 379.15 m/z, found 380.16 [M+H]⁺.

### Step 2: N-(2-(3-(1,3-dioxolan-2-yl)-4-((4-methoxybenzyl)oxy)phenyl)pyrimidin-4-yl)-S-isopropyl-8-(3-((methylsulfonyl)methyl)azetidin-1-yl)isoquinolin-3-amine

2-[3-(1,3-dioxolan-2-yl)-4-[(4-methoxyphenyl)methoxy]phenyl]pyrimidin-4-amine (80 mg, 0.211 mmol, 1 equiv) and dioxane (3 mL, 35.412 mmol) were added into 40 ml reaction vessel, a stir bar, then Cs₂CO₃ (137.40 mg, 0.422 mmol, 2 equiv), Ephos Pd G4 (19.37 mg, 0.021 mmol, 0.1 equiv) and 3-chloro-S-isopropyl-8-[3-(methanesulfonylmethyl)azetidin-1-yl]isoquinoline (74.40 mg, 0.211 mmol, 1 equiv) were added to a 40 ml reaction vessel and stirred until homogeneous. Nitrogen protection throughout the reaction process. The reaction was stirred overnight at 100° C. desired product was detected by LCMS. The resulting mixture was extracted with EA20 ml. The combined organic layers, dried over anhydrous sodium sulfate. After filtration, the filtrate was concentrated under reduced pressure. The residue was purified by reverse flash chromatography with the following conditions: column, C18 silica gel; mobile phase, water in ACN, 20% to 40% gradient in 10 min; detector, UV 254 nm. to afford N-{2-[3-(1,3-dioxolan-2-yl)-4-[(4-methoxyphenyl)methoxy]phenyl]pyrimidin-4-yl}-S-isopropyl-8-[3-(methanesulfonylmethyl)azetidin-1-yl]isoquinolin-3-amine (40 mg, 27.26%) as a yellow solid. MS (ESI) calcd. for C₃₈H₄₁N₅O₆S, 695.28 m/z, found 696.25 [M+H]⁺.

### Step 3: 2-hydroxy-S-(4-((5-isopropyl-8-(3-((methylsulfonyl)methyl)azetidin-1-yl)isoquinolin-3-yl)amino)pyrimidin-2-yl)benzaldehyde TFA salt

TFA (0.15 mL) and DCM (1 mL) were added into 40 ml reaction vessel and stir well, then N-{2-[3-(1,3-dioxolan-2-yl)-4-[(4-methoxyphenyl)methoxy]phenyl]pyrimidin-4-yl}-5-isopropyl-8-[3-(methanesulfonylmethyl)azetidin-1-yl]isoquinolin-3-amine (30 mg, 0.043 mmol, 1 equiv) was added into 40 ml reaction vessel, a stir barThe reaction was stirred 20 min at rt. The desired product was detected by LCMS. The resulting mixture was concentrated under reduced pressure. The crude product was purified by Prep-HPLC with Column: XBridge Prep OBD C18 Column, 30*150 mm, 5 μm; Mobile Phase A: Water (0.05% TFA), Mobile Phase B: ACN; Flow rate: 60 mL/min; Gradient: 29% B to 51% B in 10 min, 51% B; Wave Length: 254/220 nm; RT1(min): 10.05; Number Of Runs: 0, to afford 2-hydroxy-S-(4-((5-isopropyl-8-(3-((methylsulfonyl)methyl)azetidin-1-yl)isoquinolin-3-yl)amino)pyrimidin-2-yl)benzaldehyde TFA salt (5 mg, 20.42%) as a red solid. MS (ESI) calcd. for C₂₈H₂₉N₅O₄S, 531.19 m/z, found 532.20[M+H]⁺. ¹H NMR (300 MHz, DMSO-d6) δ 11.45 (s, 1H), 10.64 (s, 1H), 10.38 (s, 1H), 9.13 (s, 1H), 8.69 (d, J=2.3 Hz, 2H), 8.58-8.50 (m, 2H), 8.47 (d, J=6.1 Hz, 1H), 7.46 (d, J=8.0 Hz, 1H), 7.41-7.29 (m, 1H), 7.24-7.11 (m, 1H), 6.46 (d, J=8.1 Hz, 1H), 4.42 (t, J=7.6 Hz, 2H), 3.99 (t, J=6.9 Hz, 2H), 3.35-3.25 (m, 3H), 3.03 (s, 3H), 1.31 (d, J=6.7 Hz, 6H).

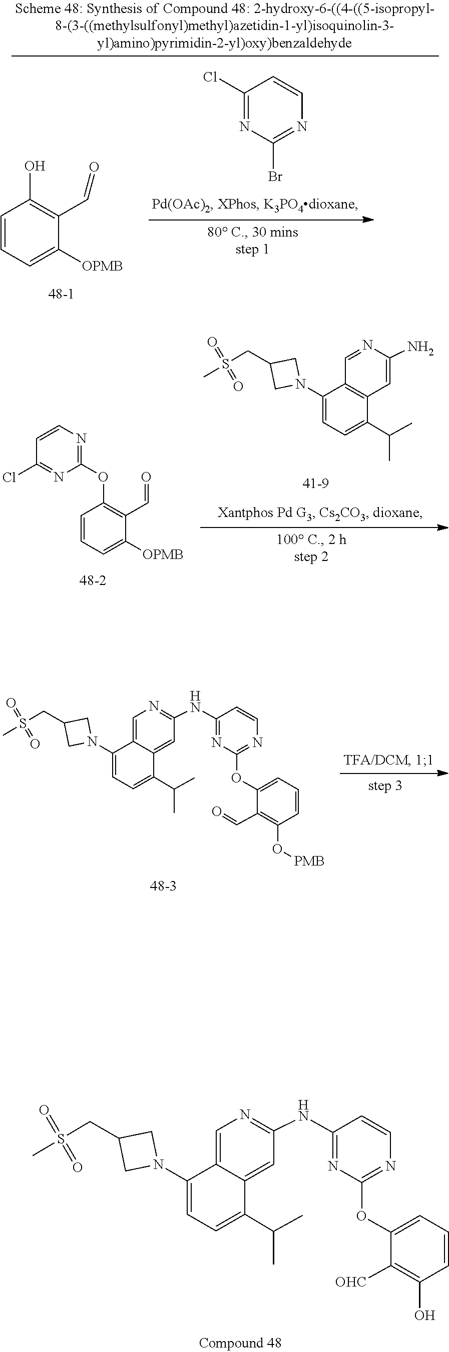

### Step 1: Synthesis of 2-((4-chloropyrimidin-2-yl)oxy)-6-((4-methoxybenzyl)oxy)benzaldehyde

To a 40 mL microwave tube equipped with a stirring bar were added 2-hydroxy-6-((4-methoxybenzyl)oxy)benzaldehyde (500 mg, 1.93 mmol), 2-bromo-4-chloropyrimidine (370.5 mg, 1.93 mmol), K₃PO₄ (818.3 mg, 3.86 mmol) and 10 mL 1,4-dioxane, followed by addition of Pd(OAc)₂ (43.3 mg, 0.193 mmol) and XPhos (183.7 mg, 0.386 mmol). The resulting solution was stirred at 80° C. for 0.5 h. The reaction progress was monitored by LCMS, and it showed the reaction was completed. The resulting mixture was extracted with EA (2×100 mL), dried over anhydrous Na₂SO₄. After filtration, the filtrate was concentrated under reduced pressure. The residue was concentrated in vacuo to afford yellow crude. The crude was purified by reverse phase column eluted with CH₃CN and water (0.05% TFA) to afford 2-((4-chloropyrimidin-2-yl)oxy)-6-((4-methoxybenzyl)oxy)benzaldehyde as a yellow solid (80 mg, 12.5% yield). MS (ESI) calcd. for C₁₉H₁₅ClN₂O₄, 370.07 m/z, found 371.10[M+H]⁺.

### Step 2: Synthesis of 2-((4-((5-isopropyl-8-(3-((methylsulfonyl)methyl)azetidin-1-yl)isoquinolin-3-yl)amino)pyrimidin-2-yl)oxy)-6-((4-methoxybenzyl)oxy)benzaldehyde

To a 12 mL microwave tube equipped with a stirring bar were added 2-((4-chloropyrimidin-2-yl)oxy)-6-((4-methoxybenzyl)oxy)benzaldehyde (80 mg, 0.22 mmol), Key Int. 1-C(87.9 mg, 0.264 mmol), Cs₂CO₃ (143.44 mg, 0.44 mmol) and 4 mL of 1,4-dioxane, followed by addition of XantPhos-Pd G3 (19.1 mg, 0.022 mmol). The resulting solution was stirred at 100° C. for 2 h. The reaction progress was monitored by LCMS, and it showed the reaction was completed. The resulting mixture was extracted with EA (2×100 mL), dried over anhydrous Na₂SO₄. After filtration, the filtrate was concentrated under reduced pressure. The residue was concentrated in vacuo to afford yellow crude. The crude was purified by reverse phase column eluted with CH₃CN and water (0.05% TFA) to afford 2-((4-((5-isopropyl-8-(3-((methylsulfonyl)methyl)azetidin-1-yl)isoquinolin-3-yl)amino)pyrimidin-2-yl)oxy)-6-((4-methoxybenzyl)oxy)benzaldehyde as a yellow solid (20 mg, 13.6% yield). MS (ESI) calcd. for C₃₆H₃₇N₅O₆S, 667.25 m/z, found 668.20[M+H]⁺.

### Step 3: Synthesis of 2-hydroxy-6-((4-((5-isopropyl-8-(3-((methylsulfonyl)methyl)azetidin-1-yl)isoquinolin-3-yl)amino)pyrimidin-2-yl)oxy)benzaldehyde

To a 25 ml round-bottomed flask equipped with a stirring bar were added 2-((4-((5-isopropyl-8-(3-((methylsulfonyl)methyl)azetidin-1-yl)isoquinolin-3-yl)amino)pyrimidin-2-yl)oxy)-6-((4-methoxybenzyl)oxy)benzaldehyde (20 mg, 0.029 mmol) and 2 mL TFA. The resulting solution was stirred at rt for 5 mins. The reaction progress was monitored by LCMS, and it showed the reaction was completed. The resulting mixture was purified by reverse phase column eluted with CH₃CN and H₂O. After lyophilized to afford the 2-hydroxy-6-((4-((5-isopropyl-8-(3-((methylsulfonyl)methyl)azetidin-1-yl)isoquinolin-3-yl)amino)pyrimidin-2-yl)oxy)benzaldehyde as a yellow solid (0.6 mg, 3.1% yield). MS (ESI) calcd. for C₂₈H₂₉N₅O₅S, 547.19 m/z, found 548.20 [M+H]⁺. ¹H-NMR (300 MHz, DMSO-d⁶) δ (ppm): 10.28 (s, 1H), 9.76 (s, 1H), 8.98 (s, 1H), 8.45 (s, 1H), 8.40-8.41 (m, 2H), 7.40-7.42 (m, 1H), 7.31-7.34 (m, 1H), 6.83-6.86 (m, 1H), 6.80-6.82 (m, 1H), 6.53-6.56 (m, 1H), 6.34-6.37 (m, 1H), 4.32-4.37 (m, 2H), 3.91-3.94 (m, 2H), 3.54-3.57 (m, 3H), 3.43-3.47 (m, 1H), 3.01 (s, 3H), 1.16 (s, 6H).

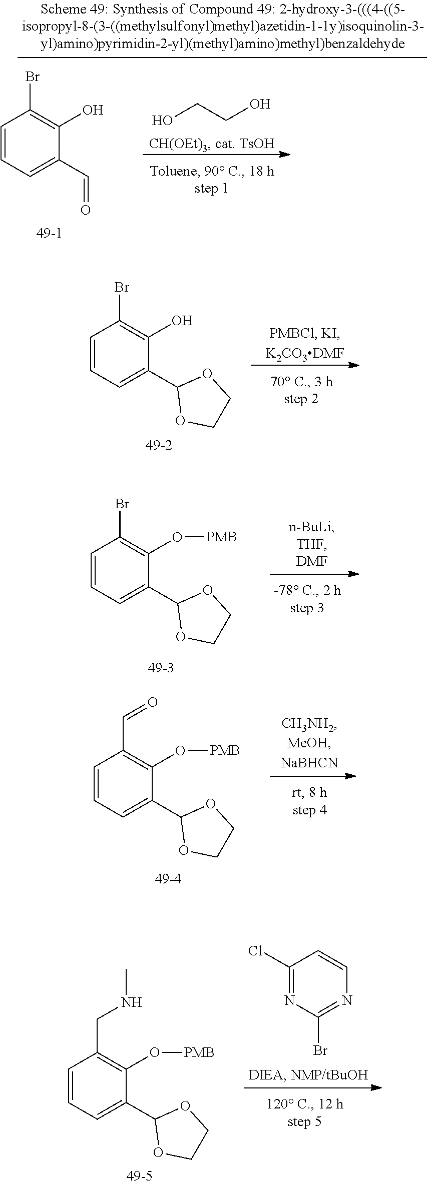

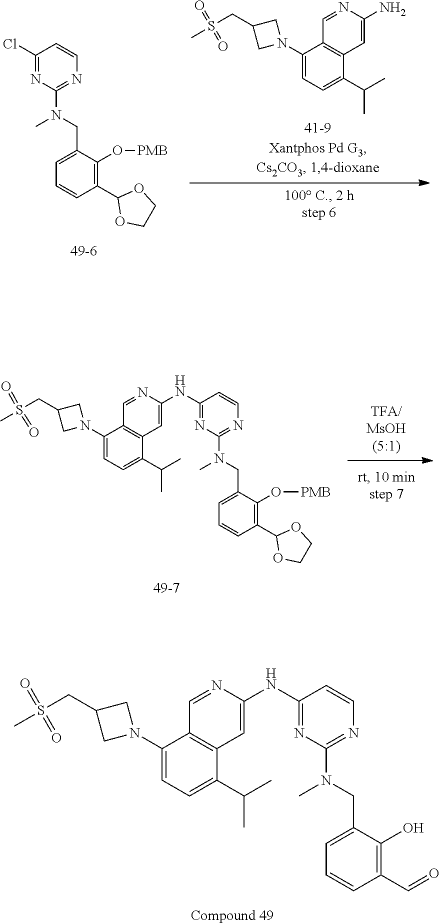

### Step 1: Synthesis of 2-bromo-6-(1,3-dioxolan-2-yl)phenol

To a 250 ml round-bottomed flask equipped with a stirring bar were added 3-bromo-2-hydroxybenzaldehyde (5 g, 24.873 mmol) and 100 mL Toluene, followed by addition of ethane-1,2-diol (10 g, 161.114 mmol), CH(OEt)₃ (12 g, 80.971 mmol) and TsOH (0.5 g, 2.904 mmol). The resulting solution was stirred at 90° C. for 15 h. The reaction progress was monitored by LCMS, and it showed the reaction was completed. The resulting mixture was extracted with EA (3×80 mL), dried over anhydrous Na₂SO₄. After filtration, the filtrate was concentrated under reduced pressure. The residue was concentrated to dryness in vacuo to give a yellow oil. The yellow oil was purified by silica gel chromatography (0-10% EA/PE) to afford the 2-bromo-6-(1,3-dioxolan-2-yl)phenol as a white oil (5 g, 53.71I % yield). MS (ESI) calcd. for C₉H₉BrO₃, 243.97 m/z, found 245.00 [M+H]⁺.

### Step 2: Synthesis of 2-(3-bromo-2-((4-methoxybenzyl)oxy)phenyl)-1,3-dioxolane

To a 100 ml round-bottomed flask equipped with a stirring bar were added 2-bromo-6-(1,3-dioxolan-2-yl)phenol (4 g, 16.322 mmol) and 80 mL DMF, followed by addition of PMBCl (3.5 g, 22.349 mmol), K₂CO₃ (7 g, 50.283 mmol), K1 (0.3 g, 1.807 mmol). The resulting solution was stirred at 70° C. for 3 h. The reaction progress was monitored by LCMS, and it showed the reaction was completed. The resulting mixture was extracted with EA (3×80 mL), dried over anhydrous Na₂SO₄. After filtration, the filtrate was concentrated under reduced pressure. The residue was concentrated to dryness in vacuo to give a yellow oil. The yellow oil was purified by silica gel chromatography (25% EA/PE) to afford the 2-(3-bromo-2-((4-methoxybenzyl)oxy)phenyl)-1,3-dioxolane as a yellow oil (3.5 g, 52.84% yield). MS (ESI) calcd. for C₁₇H₁₇BrO₄, 364.03 m/z, found 265.05 [M+H]⁺.

### Step 3: Synthesis of 3-(1,3-dioxolan-2-yl)-2-((4-methoxybenzyl)oxy)benzaldehyde

To a 50 ml round-bottomed flask equipped with a stirring bar were added 2-(3-bromo-2-((4-methoxybenzyl)oxy)phenyl)-1,3-dioxolane (1 g, 2.738 mmol) and 15 mL THF, followed by addition of n-BuLi (2.2 g, 34.343 mmol) at −78° C. for 0.5 h under N₂. Into a 50 ml round-bottomed flask were added DMF at −78° C. The mixture was stirred at rt for 1.5 h under a N2.

The resulting mixture was stirred for extracted with EA (3×30 mL), dried over anhydrous Na₂SO₄. After filtration, the filtrate was concentrated under reduced pressure. The residue was concentrated to dryness in vacuo to give a yellow oil. The yellow oil was purified by silica gel chromatography (25% EA/PE) to afford the 3-(1,3-dioxolan-2-yl)-2-((4-methoxybenzyl)oxy)benzaldehyde as a yellow oil (400 mg, 41.83% yield). MS (ESI) calcd. for C18H₁₈05, 314.12 m/z, found 315.15 [M+H]⁺.

### Step 4: Synthesis of 1-(3-(1,3-dioxolan-2-yl)-2-((4-methoxybenzyl)oxy)phenyl)-N-methylmethanamine

To a 100 ml round-bottomed flask equipped with a stirring bar were added 1-(3-(1,3-dioxolan-2-yl)-2-((4-methoxybenzyl)oxy)phenyl)-N-methylmethanamine (2 g, 6.363 mmol) and 30 mL MeOH, followed by addition of CH₃NH₂ (2.1 g, 67.615 mmol), NaBHCN (2.4 g, 39.454 mmol). The resulting solution was stirred at rt for 8 h. The reaction progress was monitored by LCMS, and it showed the reaction was completed. The resulting mixture was extracted with EA (3 * 50 mL), dried over anhydrous Na₂SO₄. After filtration, the filtrate was concentrated under reduced pressure. The residue was concentrated to dryness in vacuo to give a yellow oil. The yellow oil was purified by silica gel chromatography (25% EA/PE) to afford the 1-(3-(1,3-dioxolan-2-yl)-2-((4-methoxybenzyl)oxy)phenyl)-N-methylmethanamine as a yellow solid (600 mg, 22.090% yield). MS (ESI) calcd. for C₁₉H₂₃NO₄, 329.16 m/z, found 330.20 [M+H]⁺.

### Step 5: Synthesis of N-(3-(1,3-dioxolan-2-yl)-2-((4-methoxybenzyl)oxy)benzyl)-4-chloro-N-methylpyrimidin-2-amine

To a 20 ml round-bottomed flask equipped with a stirring bar were addedl-(3-(1,3-dioxolan-2-yl)-2-((4-methoxybenzyl)oxy)phenyl)-N-methylmethanamine (480 mg, 1.457 mmol) and 5 mL t-BuOH, followed by addition of 2-bromo-4-chloropyrimidine (185 mg, 0.956 mmol), DIEA (440 mg, 3.404 mmol). The resulting solution was stirred at 120° C. for 12 h. The reaction progress was monitored by LCMS, and it showed the reaction was completed. The resulting mixture was extracted with EA (3 * 100 mL), dried over anhydrous Na₂SO₄. After filtration, the filtrate was concentrated under reduced pressure. The residue was concentrated to dryness in vacuo to give a yellow oil. The yellow oil was purified by reverse phase column (45% ACN) to afford the N-(3-(1,3-dioxolan-2-yl)-2-((4-methoxybenzyl)oxy)benzyl)-4-chloro-N-methylpyrimidin-2-amine a yellow solid (100 mg, 10.39% yield). MS (ESI) calcd. for C₂₃H₂₄ClN₃O₄, 441.15 m/z, found 442.20 [M+H]⁺.

### Step 6: Synthesis of N2-(3-(1,3-dioxolan-2-yl)-2-((4-methoxybenzyl)oxy)benzyl)-N4-(5-isopropyl-8-(3-((methylsulfonyl)methyl)azetidin-1-yl)isoquinolin-3-yl)-N2-methylpyrimidine-2,4-diamine

To a 50 ml round-bottomed flask equipped with a stirring bar were added N-(3-(1,3-dioxolan-2-yl)-2-((4-methoxybenzyl)oxy)benzyl)-4-chloro-N-methylpyrimidin-2-amine (90 mg, 0.204 mmol) and 2 mL dioxane, followed by addition of 5-isopropyl-8-(3-((methylsulfonyl)methyl)azetidin-1-yl)isoquinolin-3-amine (75 mg, 0.225 mmol), Xantphos Pd G3 (19 mg, 0.020 mmol), Cs₂CO₃ (150 mg, 0.459 mmol). The resulting solution was stirred at 100° C. for 2 h under the N2. The resulting mixture was extracted with EA (3 * 50 mL), dried over anhydrous Na₂SO₄. After filtration, the filtrate was concentrated under reduced pressure. The residue was concentrated to dryness in vacuo to give a yellow oil. The yellow oil was purified by reverse phase column (45% ACN) to afford the N2-(3-(1,3-dioxolan-2-yl)-2-((4-methoxybenzyl)oxy)benzyl)-N4-(5-isopropyl-8-(3-((methylsulfonyl)methyl)azetidin-1-yl)isoquinolin-3-yl)-N2-methylpyrimidine-2,4-diamine a yellow solid (60 mg, 25.29% yield).

MS (ESI) calcd. for C₄₀H₄₆N₆O₆S, 738.32 m/z, found 739.40 [M+H]⁺.

### Step 7: Synthesis of 2-hydroxy-3-(((4-((5-isopropyl-8-(3-((methylsulfonyl)methyl)azetidin-1-yl)isoquinolin-3-yl)amino)pyrimidin-2-yl)(methyl)amino)methyl)benzaldehyde

N2-{[3-(1,3-dioxolan-2-yl)-2-[(4-methoxyphenyl)methoxy]phenyl]methyl}-N4-{5-isopropyl-8-[3-(methanesulfonylmethyl)azetidin-1-yl]isoquinolin-3-yl}-N2-methylpyrimidine-2,4-diamine (55 mg, 0.074 mmol, 1 equiv), a stir bar, trifluoroacetic acid (1 mL) and methanesulfonic acid (0.2 mL) were added to a 50-mL round-bottom flask and stirred until homogeneous. The resulting mixture was stirred at rt for 10 min. The residue was concentrated to dryness in vacuo to give a nearly yellow viscous oil. The crude product was then purified by Prep-HPLC with the following conditions: Column: XBridge Shield RP18 OBD Column, 30*150 mm, 5 μm; Mobile Phase A: Water(10 mmol/L NH₄HCO₃+0.1⁰% NH₃·H₂O), Mobile Phase B: ACN; Flow rate: 60 mL/min; Gradient: 51% B to 65% B in 9 min, 65% B; Wave Length: 254/220 nm; RT1(min): 9; Number Of Runs: 0. After lyophilization, it was afforded 2-hydroxy-3-(((4-((5-isopropyl-8-(3-((methylsulfonyl)methyl)azetidin-1-yl)isoquinolin-3-yl)amino)pyrimidin-2-yl)(methyl)amino)methyl)benzaldehyde (1.0 mg, 2.23%) as a yellow solid. MS (ESI) calcd. for C₃₀H₃₄N₆O₄S, 574.24 m/z, found 575.25 [M+H]⁺. ¹H NMR (300 MHz, DMSO-d6) δ (ppm): 10.20˜10.30 (m, 1H), 10.03-11.06 (m, 1H), 9.00-9.08 (m, 1H), 8.62-8.80 (m, 1H), 8.01-8.10 (m, 1H), 7.50-7.70 (m, 2H), 7.31-7.44 (m, 1H), 6.89-7.00 (m, 1H), 6.50-6.60 (m, 1H), 6.33-6.43 (m, 1H), 4.71-4.93 (m, 2H), 4.32-4.40 (m, 2H), 3.90-4.00 (m, 2H), 3.51-3.60 (m, 2H), 3.20-3.33 (m, 5H), 3.01 (s, 3H), 1.15-1.30 (m, 6H).

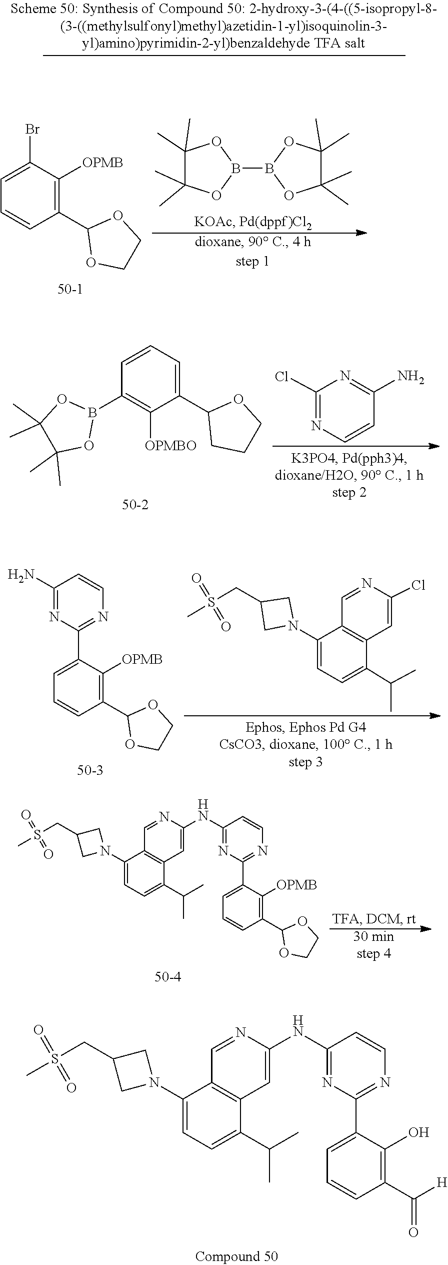

### Step 1: Synthesis of 2-(3-(1,3-dioxolan-2-yl)-2-((4-methoxybenzyl)oxy)phenyl)-4,4,5,5-tetramethyl-1,3,2-dioxaborolane

2-{3-bromo-2-[(4-methoxyphenyl)methoxy]phenyl}-1,3-dioxolane (1 g, 2.738 mmol, 1 equiv) and dioxane (8 mL) were added to the 100 ml reaction vessel, a stir bar, then added KOAc (0.81 g, 8.214 mmol, 3 equiv); Pd(dppf)Cl₂ (0.20 g, 0.274 mmol, 0.1 equiv) and bis(pinacolato)diboron (0.70 g, 2.738 mmol, 1 equiv) to a 100 ml reaction vessel and stirred until homogeneous. The reaction was stirred 6h at 90° C. Nitrogen protection throughout the reaction process. The desired product was detected by LCMS, The reaction was quenched with water 3 ml. The aqueous layer was extracted with EA 30 mL. The combined organic layers dried over anhydrous sodium sulfate. After filtration, the filtrate was concentrated under reduced pressure.

The residue was purified by silica gel column chromatography, eluted with PE:EA(4:1) to afford 2-[3-(1,3-dioxolan-2-yl)-2-[(4-methoxyphenyl)methoxy]phenyl]-4,4,5,5-tetramethyl-1,3,2-dioxaborolane (1 g, 88.58%) as a yellow oil. MS (ESI) calcd. for C₂₃H₂₉BO₆, 412.21m/z, found 413.20 [M+H]⁺.

### Step 2: Synthesis of 2-(3-(1,3-dioxolan-2-yl)-2-((4-methoxybenzyl)oxy)phenyl)pyrimidin-4-amine

2-chloropyrimidin-4-amine (150 mg, 1.158 mmol, 1 equiv), dioxane (2.4 mL, 28.330 mmol), H₂O (0.6 mL, 33.306 mmol) were added into 40 ml reaction vessel, a stir bar, then K₃PO₄ (491.54 mg, 2.316 mmol, 2 equiv), Pd(PPH₃)₄(133.80 mg, 0.116 mmol, 0.1 equiv) and 2-[3-(1,3-dioxolan-2-yl)-2-[(4-methoxyphenyl)methoxy]phenyl]-4,4,5,5-tetramethyl-1,3,2-dioxaborolane (477.37 mg, 1.158 mmol, 1 equiv) were added to a 40 ml reaction vessel and stirred until homogeneous. Nitrogen protection throughout the reaction process. The reaction was stirred overnight at 90° C. Desired product was detected by LCMS. The reaction was quenched with water 3 ml. The aqueous layer was extracted with EA 30 ml. The combined organic layers dried over anhydrous sodium sulfate. After filtration, the filtrate was concentrated under reduced pressure. The residue was purified by silica gel column chromatography, eluted with PE:EA (9:1) to afford 2-[3-(1,3-dioxolan-2-yl)-2-[(4-methoxyphenyl)methoxy]phenyl]pyrimidin-4-amine (100 mg, 22.76%) as a yellow oil. MS (ESI) calcd. for C₂₁H₂₁N₃O₄, 379.15m/z, found 380.16 [M+H]⁺.

### Step 3: Synthesis of N-(2-(3-(1,3-dioxolan-2-yl)-2-((4-methoxybenzyl)oxy)phenyl)pyrimidin-4-yl)-S-isopropyl-8-(3-((methylsulfonyl)methyl)azetidin-1-yl)isoquinolin-3-amine

2-[3-(1,3-dioxolan-2-yl)-2-[(4-methoxyphenyl)methoxy]phenyl]pyrimidin-4-amine (80 mg, 0.211 mmol, 1 equiv) and dioxane (3 mL) were added into 40 ml reaction vessel, a stir bar, then Cs₂CO₃ (137.40 mg, 0.422 mmol, 2 equiv), Ephos Pd G4 (19.37 mg, 0.021 mmol, 0.1 equiv), Ephos (11.28 mg, 0.021 mmol, 0.1 equiv) and 3-chloro-S-isopropyl-8-[3-(methanesulfonylmethyl)azetidin-1-yl]isoquinoline (74.40 mg, 0.211 mmol, 1 equiv) were added to a 40 ml reaction vessel and stirred until homogeneous. Nitrogen protection throughout the reaction process. The reaction was stirred overnight at 90° C. The desired product was detected by LCMS. The reaction was quenched with 2 mL water. The resulting mixture was extracted with EA (3×20 mL). The combined organic layers were dried over anhydrous sodium sulfate. After filtration, the filtrate was concentrated under reduced pressure. The residue was purified by reverse flash chromatography with the following conditions: column, C18 silica gel; mobile phase, water in TFA and ACN, 10% to 50% gradient in 10 min; detector, UV 254 nm. To afford N-{2-[3-(1,3-dioxolan-2-yl)-2-[(4-methoxyphenyl)methoxy]phenyl]pyrimidin-4-yl}-S-isopropyl-8-[3-(methanesulfonylmethyl)azetidin-1-yl]isoquinolin-3-amine) as a yellow solid. MS (ESI) calcd. for C₃₈H₄₁N₅O₆S, 695.28 m/z, found 696.23 [M+H]⁺.

### Step 4: Synthesis of 2-hydroxy-3-(4-((5-isopropyl-8-(3-((methylsulfonyl)methyl)azetidin-1-yl)isoquinolin-3-yl)amino)pyrimidin-2-yl)benzaldehyde TFA salt

TFA (0.15 mL) and DCM (1 mL) were added into 40 ml reaction vessel and stir well, then N-{2-[3-(1,3-dioxolan-2-yl)-2-[(4-methoxyphenyl)methoxy]phenyl]pyrimidin-4-yl}-S-isopropyl-8-[3-(methanesulfonylmethyl)azetidin-1-yl]isoquinolin-3-amine (20 mg, 0.029 mmol, 1 equiv) was added into 40 ml reaction vessel, a stir bar. The reaction was stirred 20 min at rt desired product was detected by LCMS. The resulting mixture was concentrated under reduced pressure. The crude product was purified by Prep-HPLC with Column: XBridge Prep OBD C18 Column, 30*150 mm, 5 μm; Mobile Phase A: Water (0.05% TFA), Mobile Phase B: ACN; Flow rate: 60 mL/min; Gradient: 30% B to 51% B in 10 min, 51% B; Wave Length: 254/220 nm; RT1(min): 10.05; Number Of Runs: 0 to afford 2-hydroxy-3-(4-((5-isopropyl-8-(3-((methylsulfonyl)methyl)azetidin-1-yl)isoquinolin-3-yl)amino)pyrimidin-2-yl)benzaldehyde TFA salt (0.6 mg, 3.14%) as a red solid. MS (ESI) calcd. for C₂₈H₂₉N₅O₄S, 531.19 m/z, found 532.20 [M+H]⁺. ¹H NMR (300 MHz, DMSO-d₆) δ 10.74 (s, 1H), 10.52 (s, 1H), 9.15 (s, 1H), 8.77 (d, J=7.6 Hz, 1H), 8.52 (d, J=6.1 Hz, 1H), 7.87 (d, J=7.3 Hz, 1H), 7.50 (d, J=7.9 Hz, 1H), 7.40 (s, 1H), 7.17-7.05 (m, 2H), 6.94 (s, 1H), 6.49 (d, J=8.1 Hz, 1H), 4.43 (t, J=7.6 Hz, 2H), 4.00 (t, J=6.8 Hz, 2H), 3.65-3.59 (m, 4H), 3.03 (s, 3H), 1.37 (d, J=6.8 Hz, 6H).

### Example 4: Representative Compound Syntheses Following General Schemes C₁-C₂

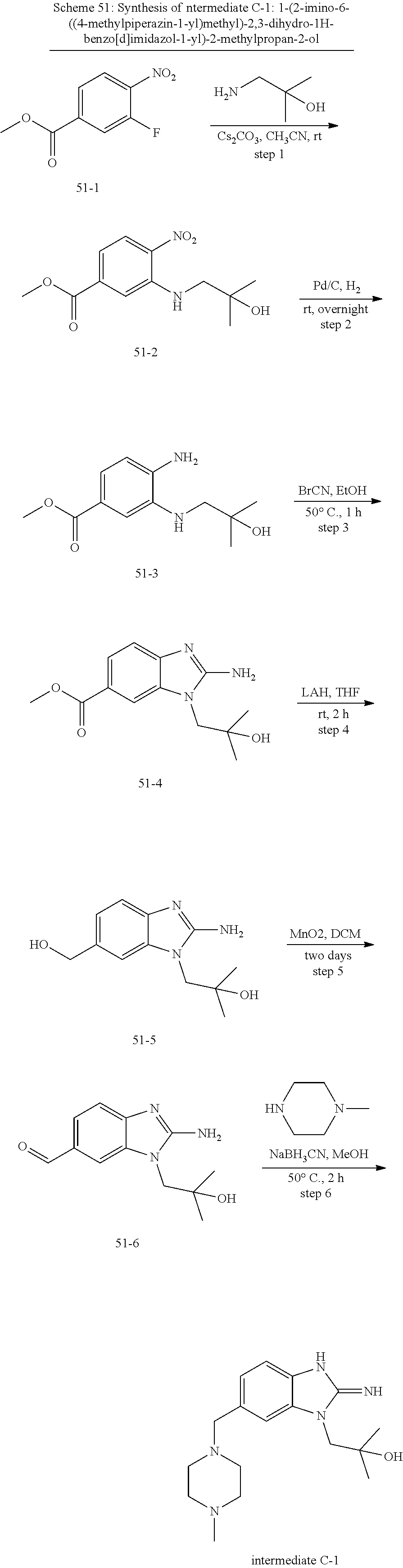

### Step 1: Synthesis of methyl 3-((2-hydroxy-2-methylpropyl)amino)-4-nitrobenzoate

To a 1000 ml round-bottomed flask equipped with a stirring bar were added methyl 3-fluoro-4-nitrobenzoate (30 g, 150.7 mmol) and 300 mL ACN, followed by addition of 1-amino-2-methylpropan-2-ol (26.8 g, 301.5 mmol) and Cs₂CO₃ (98 g, 301.5 mmol) at 0° C. The resulting solution was stirred at rt for 2 h. The reaction progress was monitored by LCMS, and it showed the reaction was completed. The resulting mixture was extracted with EA (3 * 50 mL), The combined organic layers were washed with water (20×3), dried over anhydrous Na₂SO₄. After filtration, the filtrate was concentrated under reduced pressure. The residue was concentrated to dryness in vacuo to give a yellow oil. The yellow oil was purified by silica gel chromatography (0-20% EA/PE) to afford the methyl 3-((2-hydroxy-2-methylpropyl)amino)-4-nitrobenzoate as a yellow solid (25 g, 60% yield). MS (ESI) calcd. for C₁₂H₁₇N₃O₃, 251.13 m/z, found 252.15 [M+H]⁺.

### Step 2: Synthesis of methyl 4-amino-3-((2-hydroxy-2-methylpropyl)amino)benzoate

methyl 4-amino-3-((2-hydroxy-2-methylpropyl)amino)benzoate (25 g, 104.916 mmol, 1 equiv), a stir bar, ethyl alcohol (400 mL) and cyanogen bromide (22.3 g, 210.532 mmol, 2 equiv) were added to a 1-L round-bottom flask and stirred until homogeneous. The resulting mixture was stirred at rt for 12 h. The residue was concentrated to dryness in vacuo to give a nearly yellow viscous oil. The yellow solid was purified by silica gel chromatography (0-10% MeOH/DCM) to afford the methyl 2-amino-i-(2-hydroxy-2-methylpropyl)-1H-benzo[d]imidazole-6-carboxylate as a yellow solid (26 g, 94.12% yield). MS (ESI) calcd. for C₁₂H₁₈N₂O₃, 238.13 m/z, found 239.15 [M+H]⁺. Step 3: Synthesis of methyl 2-amino-1-(2-hydroxy-2-methylpropyl)-1H-benzo[d]imidazole-6-carboxylate

methyl 4-amino-3-((2-hydroxy-2-methylpropyl)amino)benzoate (25 g, 104.916 mmol, 1 equiv), a stir bar, ethyl alcohol (400 mL) and cyanogen bromide (22.3 g, 210.532 mmol, 2 equiv) were added to a 1-L round-bottom flask and stirred until homogeneous. The resulting mixture was stirred at 50° C. for 1 h. The residue was concentrated to dryness in vacuo to give a nearly yellow viscous oil. The yellow solid was purified by silica gel chromatography (0-10% MeOH/DCM) to afford the methyl 2-amino-i-(2-hydroxy-2-methylpropyl)-1H-benzo[d]imidazole-6-carboxylate as a yellow solid (26 g, 94.12% yield). MS (ESI) calcd. for C₁₃H₁₇N₃O₃, 263.13 m/z, found 264.15 [M+H]⁺.

### Step 4: Synthesis of 1-(2-amino-6-(hydroxymethyl)-1H-benzo[d]imidazol-1-yl)-2-methylpropan-2-ol

methyl 2-amino-i-(2-hydroxy-2-methylpropyl)-1H-benzo[d]imidazole-6-carboxylate (26 g, 98.748 mmol, 1 equiv), a stir bar, THF (400 mL) and LAH (11 g, 289.855 mmol, 2 equiv) were added to a 1-L round-bottom flask and stirred until homogeneous. The resulting mixture was stirred at 40° cfor 1 h. The resulting mixture was filtered, the filter cake was washed with water (3×100 mL). The filtrate was concentrated under reduced pressure. It is to afford the 1-(2-amino-6-(hydroxymethyl)-1H-benzo[d]imidazol-1-yl)-2-methylpropan-2-ol as a white solid (20 g, 86.08% yield). MS (ESI) calcd. for C₁₂H₁₇N₃O_{2-, 235.13} m/z, found 236.15 [M+H]⁺.

### Step 5: Synthesis of 2-amino-1-(2-hydroxy-2-methylpropyl)-1H-benzo[d]imidazole-6-carbaldehyde

To a 1000 ml round-bottomed flask equipped with a stirring bar were added 1-(2-amino-6-(hydroxymethyl)-1H-benzo[d]imidazol-1-yl)-2-methylpropan-2-ol (15 g, 63.752 mmol) in 200 mL ACN and 200 H₂O, followed by addition of MnO₂ (22 g, 253.060 mmol). The resulting solution was stirred at 50° C. for 48 h. The resulting mixture was filtered, the filter cake was washed with EA (3×20 mL). The filtrate was concentrated under reduced pressure. The residue was purified by silica gel column chromatography, eluted with EA/PE (20%) to afford the 2-amino-i-(2-hydroxy-2-methylpropyl)-1H-benzo[d]imidazole-6-carbaldehyde a yellow solid (2 g, 49.51% yield). MS (ESI) calcd. for C₁₂H₁₅N₃O_{2-, 233.12} m/z, found 234.15 [M+H]⁺.

### Step 6: Synthesis of 1-(2-imino-6-((4-methylpiperazin-1-yl)methyl)-2,3-dihydro-1H-benzo[d]imidazol-1-yl)-2-methylpropan-2-ol

To a 250 ml round-bottomed flask equipped with a stirring bar were added 2-amino-3-(2-hydroxy-2-methylpropyl)-1,3-benzodiazole-S-carbaldehyde (8 g, 34.295 mmol) in 20 mL Acetic Acid and 100 MeOH, followed by addition of 1-methylpiperazine (3.7 g, 36.939 mmol), NaBH₃CN (3.2 g, 50.923 mmol). The resulting solution was stirred at 50° C. for 2 h. The reaction mixture was concentrated directly under reduced pressure to obatin a 10 g of yellow oil. The yellow oil obtained was purified by reverse phase column (30% ACN) to afford the 1-(2-amino-6-((4-methylpiperazin-1-yl)methyl)-1H-benzo[d]imidazol-1-yl)-2-methylpropan-2-ol a off-white solid (5 g, 27.14% yield). MS (ESI) calcd. for C₁₇H₂₇N₅O, 317.22 m/z, found 318.20 [M+H]⁺.

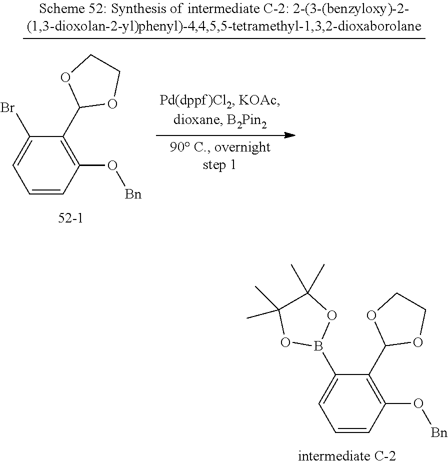 [SMILES: Brc1cccc(OCc2ccccc2)c1C1OCCO1 ; CC1(C)OB(c2cccc(OCc3ccccc3)c2C2OCCO2)OC1(C)C]

### Step 1: Synthesis of 2-[3-(benzyloxy)-2-(1,3-dioxolan-2-yl)phenyl]-4,4,5,5-tetramethyl-1,3,2-dioxaborolane

A suspension of 2-[2-(benzyloxy)-6-bromophenyl]-1,3-dioxolane (2 g, 5.967 mmol, 1 equiv), octamethyl-[1,4,2,3]dioxadiborinino[2,3-b][1,4,2,3]dioxadiborinine (1.67 g, 6.564 mmol, 1.1 equiv), Pd(dppf)Cl₂ (0.22 g, 0.298 mmol, 0.05 equiv) and KOAc (1.76 g, 17.901 mmol, 3 equiv) in dioxane (20 mL) was stirred overnight at 90° C. under nitrogen atmosphere.

After cooled down to rt, the reaction was quenched by the addition of NH₄Cl aq. The resulting mixture was extracted with EA (5 mL×3). The combined organic layers were washed with water (5 mL×3), dried over anhydrous Na₂SO₄. After filtration, the filtrate was concentrated under reduced pressure. The residue was purified by silica gel column (0-10% PE/EA) giving 2-[3-(benzyloxy)-2-(1,3-dioxolan-2-yl)phenyl]-4,4,5,5-tetramethyl-1,3,2-dioxaborolane (1.48 g, 64.89% yield) as a light yellow solid. LC/MS: MS (ESI) calcd. for C₂₂H₂₇BO₅: 382.19. Found: 383.10 [M+H]⁺.

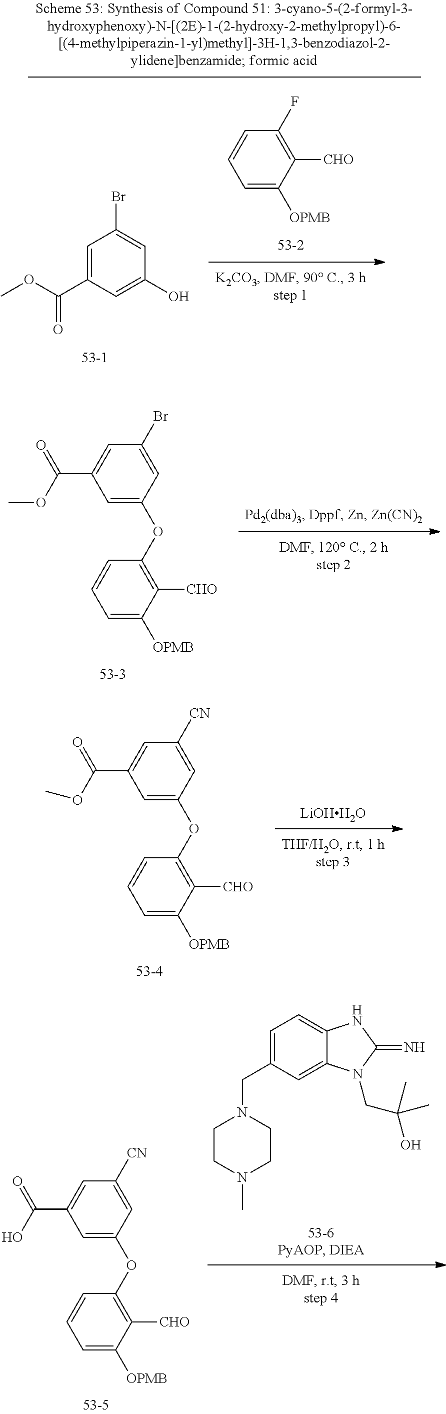

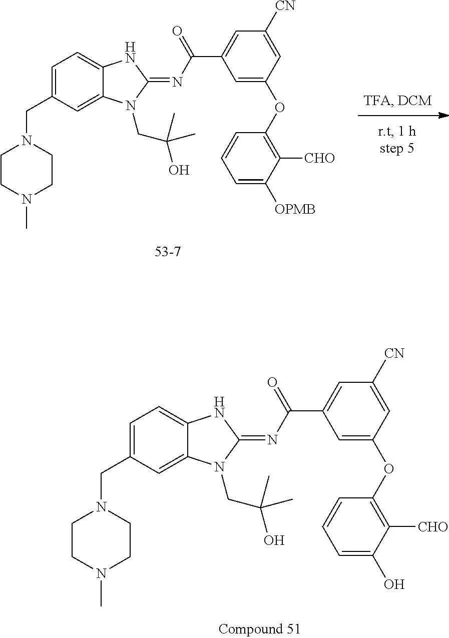

### Step 1: Synthesis of methyl 3-bromo-S-{2-formyl-3-[(4-methoxyphenyl)methoxy]phenoxy}benzoate

Methyl 3-bromo-S-hydroxybenzoate (1.0 g, 4.32 mmol, 1.0 eq.), 2-fluoro-6-[(4-methoxyphenyl)methoxy]benzaldehyde (1.2 g, 4.76 mmol, 1.1 eq.), K₂CO₃ (1.8 g, 12.98 mmol, 3.0 eq.) and DMF (20 mL) were added to a 100 mL round-bottom flask with a stir bar and stirred at 90° C. for 3 h under N₂. LCMS showed the reaction was completed. The mixture was diluted with ethyl acetate (50 mL), washed with water (10 mL×3) and brine (20 mL), dried over anhydrous sodium sulfate, and concentrated in vacuum. The residue was purified by silica gel column with EA/PE (0-35%) to afford methyl 3-bromo-S-{2-formyl-3-[(4-methoxyphenyl)methoxy]phenoxy}benzoate (1.8 g 55.7% yield) as yellow oil. MS (ESI) calcd. for C₂₃H₁₉BrO₆, 470.04 m/z, found 493.10 [M+Na]⁺. Step 2: Synthesis of methyl 3-cyano-S-{2-formyl-3-[(4-methoxyphenyl)methoxy]phenoxy}benzoate

Methyl 3-bromo-S-{2-formyl-3-[(4-methoxyphenyl)methoxy]phenoxy}benzoate (1.2 g, 2.44 mmol, 1.0 eq.), Pd₂(dba)₃ (0.22 g, 0.24 mmol, 0.1 eq.), Dppf (0.27 g, 0.48 mmol, 0.2 eq.), Zn (80 mg, 1.22 mmol, 0.5 eq.), Zn(CN)₂ (0.43 g, 3.66 mmol, 1.5 eq.) and DMF (10 mL) were added to a 40 mL glass tube with a stir bar and stirred at 120° C. for 2 h under N₂. LCMS showed the reaction was completed. The mixture was diluted with H₂O (30 mL), the mixture was filtered, and the filtrate was concentrated in vacuum. The residue was purified by silica gel column with EA/PE (0-40%) to afford methyl 3-cyano-S-{2-formyl-3-[(4-methoxyphenyl)methoxy]phenoxy}benzoate (770 mg, 69.8% yield) as light yellow solid. MS (ESI) calcd. for C₂₄H₁₉NO₆, 417.12 m/z, found 440.10 [M+Na]f.

### Step 3: Synthesis of 3-cyano-S-{2-formyl-3-[(4-methoxyphenyl)methoxy]phenoxy}benzoic acid

Methyl 3-cyano-S-{2-formyl-3-[(4-methoxyphenyl)methoxy]phenoxy}benzoate (300 mg, 0.72 mmol, 1.0 eq.), LiOH·H₂O (45.2 mg, 1.08 mmol, 1.5 eq.), THF (5 mL) and H₂O (1 mL) were added to a 20 mL glass tube with a stir bar and stirred at r.t for 1 h. LCMS showed the reaction was completed. The mixture was concentrated in vacuum at 0° C. to afford 3-cyano-5-{2-formyl-3-[(4-methoxyphenyl)methoxy]phenoxy}benzoic acid (200 mg, 54.8% yield) as light yellow solid and used in the next step directly without further purification. MS (ESI) calcd. for C₂₃H₁₇NO₆, 403.10 m/z, found 402.00 [M−H]—.

### Step 4: Synthesis of 3-cyano-S-{2-formyl-3-[(4-methoxyphenyl)methoxy]phenoxy}-N-[(2E)-1-(2-hydroxy-2-methylpropyl)-6-[(4-methylpiperazin-1-yl)methyl]-3H-1,3-benzodiazol-2-ylidene]benzamide

1-{2-Imino-6-[(4-methylpiperazin-1-yl)methyl]-3H-1,3-benzodiazol-1-yl}-2-methylpropan-2-ol (80 mg, 0.25 mmol, 1.0 eq.), 3-cyano-S-{2-formyl-3-[(4-methoxyphenyl)methoxy]phenoxy}benzoic acid (132.2 mg, 0.33 mmol, 1.3 eq.), PyAOP (197.1 mg, 0.38 mmol, 1.5 eq.), DIEA (97.7 mg, 0.76 mmol, 3.0 eq.) and DMF (2 mL) were added to a 10 mL glass tube with a stir bar and stirred at r.t for 3 h. LCMS showed the reaction was completed. The mixture was diluted with ethyl acetate (30 mL), washed with water (10 mL×3) and brine (10 mL), dried over anhydrous sodium sulfate, and concentrated in vacuum to afford 3-cyano-S—{2-formyl-3-[(4-methoxyphenyl)methoxy]phenoxy}-N-[(2E)-1-(2-hydroxy-2-methylpropyl)-6-[(4-methylpiperazin-1-yl)methyl]-3H-1,3-benzodiazol-2-ylidene]benzamide (200 mg, 77.12% yield) as brown solid. The solid was used in the next step directly without further purification. MS (ESI) calcd. for C₄₀H₄₂N₆O₆, 702.32 m/z, found 703.20 [M+H]⁺.

### Step 5: Synthesis of 3-cyano-S-(2-formyl-3-hydroxyphenoxy)-N-[(2E)-1-(2-hydroxy-2-methylpropyl)-6-[(4-methylpiperazin-1-yl)methyl]-3H-1,3-benzodiazol-2-ylidene]benzamide; formic acid

3-Cyano-S-{2-formyl-3-[(4-methoxyphenyl)methoxy]phenoxy}-N-[(2E)-1-(2-hydroxy-2-methylpropyl)-6-[(4-methylpiperazin-1-yl)methyl]-3H-1,3-benzodiazol-2-ylidene]benzamide (190 mg, 0.27 mmol, 1.0 eq.), TFA (1.0 mL) and DCM (5.0 mL) were added to a 20 mL glass tube with a stir bar and stirred at rt for 1 h. LCMS showed the reaction was completed. The mixture was purified by Prep-HPLC (Column: Xselect CSH C18 OBD Column 30*150 mm 5 μm, n; Mobile Phase A: Water(0.1% FA), Mobile Phase B: ACN; Flow rate: 60 mL/min; Gradient: 17% B to 37% B in 9 min, 37% B; Wave Length: 254/220 nm; RT1(min): 8.5; Number Of Runs: 0) to afford 3-cyano-S-(2-formyl-3-hydroxyphenoxy)-N-[(2E)-1-(2-hydroxy-2-methylpropyl)-6-[(4-methylpiperazin-1-yl)methyl]-3H-1,3-benzodiazol-2-ylidene]benzamide; formic acid (1.3 mg, 24.2% yield) as white solid. MS (ESI) calcd. for C₃₂H₃₄N₆O₅, 582.26 m/z, found 583.20 [M+H]⁺. ¹H NMR (400 MHz, DMSO-d₆) δ 12.82 (s, 1H), 11.51 (s, 1H), 10.34 (s, 1H), 8.28 (t, J=1.4 Hz, 1H), 8.05-7.99 (m, 1H), 7.90-7.78 (m, 1H), 7.64-7.48 (m, 2H), 7.46 (d, J=8.1 Hz, 1H), 7.16-7.08 (m, 1H), 6.87 (d, J=8.3 Hz, 1H), 6.58 (d, J=8.1 Hz, 1H), 4.85 (s, 1H), 4.11 (s, 2H), 3.53 (s, 2H), 2.38 (s, 8H), 2.19 (s, 3H), 1.13 (s, 6H).

The following compounds were synthesized according to the preceding method or analogous methods thereto:

| Cmpd | |
|---|---|
| No. | Characterization Data |
| 52 | MS (ESI) calcd. for C₃₁H₃₅N5O₅, 557.26 m/z, found 558.30 [M + H]⁺. ¹H-NMR |
| | (300 MHz, DMSO-d₆) δ (ppm): 12.88 (s, 1H), 10.38 (s, 1H), 8.00-8.04 (m, |
| | 1H), 7.84-7.91 (m, 1H), 7.22-7.60 (m, 5H), 7.15-7.21 (m, 1H), 6.77-6.93 |
| | (m, 1H), 6.44-6.50 (m, 1H), 4.19 (s, 2H), 3.24 (s, 2H), 2.15-2.35 (m, 7H), |
| | 2.00-2.15 (m, 4H), 1.15 (s, 6H). |
| 53 | MS (ESI) calcd. for C₃₁H₃₄FN₅O₅, 575.25 m/z, found 576.30 [M + H]⁺. ¹H NMR |
| | (400 MHZ, DMSO-d₆) δ 12.79 (s, 1H), 11.51 (s, 1H), 10.34 (s, 1H), 7.74-7.67 |
| | (m, 1H), 7.65 (d, J = 2.0 Hz, 1H), 7.58 (t, J = 8.3 Hz, 2H), 7.54 (s, 1H), 7.46 (d, |
| | J = 8.1 Hz, 1H), 7.25 (dt, J = 9.5, 2.5 Hz, 1H), 7.19-7.12 (m, 1H), 6.84 (d, J = |
| | 8.4 Hz, 1H), 6.59-6.52 (m, 1H), 4.87 (s, 2H), 4.10 (s, 2H), 2.54 (s, 7H), 2.27 (s, |
| | 4H), 1.14 (s, 6H). |
| 54 | MS (ESI) calcd. for C₂₂H₃₀N₆O₃, 591.22 m/z, found 592.22 [M + H]⁺. ¹H NMR |
| | (400 MHZ, DMSO-d₆) δ (ppm): 10.38-10.30 (m, 1H), 7.99 (s, 1H), 7.82 (d, J = |
| | 2.0 Hz, 1H), 7.70 (dd, J = 8.4, 4.3 Hz, 1H), 7.62 (s, 1H), 7.53 (ddd, J = 8.3, |
| | 6.5, 3.5 Hz, 2H), 7.29 (d, J = 8.4 Hz, 1H), 6.84-6.76 (m, 1H), 6.38-6.31 (m, |
| | 1H), 4.26-4.11 (m, 2H), 3.24-2.99 (m, 5H), 2.83-2.78 (m, 3H), 1.82-1.70 |
| | (m, 6H), 1.11 (s, 6H). |
| 55 | MS (ESI) calcd. for C₃₀H₃₄N₆O₅, 558.26 m/z, found 559.10 [M + H]⁺. ¹H-NMR |
| | (300 MHz, DMSO-d₆) δ (ppm): 12.78 (s, 1H), 10.38 (s, 1H), 9.16 (s, 1H), 8.60- |
| | 8.62 (m, 1H), 8.04-8.05 (m, 1H), 7.55-7.58 (m, 2H), 7.44-7.47 (m, 1H), |
| | 7.13-7.16 (m, 1H), 6.82-6.84 (m, 1H), 6.49-6.52 (m, 1H), 4.12 (s, 2H), 3.53 |
| | (s, 2H), 2.31-2.37 (m, 8H), 2.10 (s, 3H), 1.20 (s, 6H). |
| 56 | MS (ESI) calcd. For C₂₉H₃₂N₆O₅, 544.24 m/z, found: 545.20 [M + H]⁺. ¹H |
| | NMR (300 MHz, DMSO-d₆) δ (ppm): 12.82 (s, 1H), 11.32 (s, 1H), 10.22 (s, |
| | 1H), 8.25-8.27 (m, 1H), 7.79-7.81 (m, 1H), 7.58-7.61 (m, 1H), 7.45-7.56 |
| | (m, 1H), 7.26-7.42 (m, 1H), 7.12-7.22 (m, 1H), 6.95-7.00 (m, 1H), 6.86- |
| | 6.89 (m, 1H), 6.69-6.71 (m, 1H), 4.41-4.43 (m, 2H), 3.96-4.02 (m, 2H), |
| | 3.71-3.78 (m, 2H), 3.13-3.27 (m, 2H), 2.93-3.13 (m, 2H), 2.88 (s, 3H), 1.21 |
| | (s, 6H). |
| 58 | MS (ESI) calcd. for C₃₂H₃₅N₅O₆, 585.26 m/z, found 586.40 [M + H]⁺. ¹H NMR |
| | (300 MHz, DMSO-d₆) δ 12.83 (s, 1H), 11.53 (s, 1H), 10.35 (s, 1H), 10.09 (s, |
| | 1H), 8.47 (d, J = 1.5 Hz, 1H), 8.33 (s, 1H), 8.01-8.21(m, 1H), 7.70-7.78 (m, |
| | 1H), 7.52-7.62 (m, 2H), 7.48 (d, J = 8.1 Hz, 1H), 7.40 (s, 1H), 7.27 (s, 1H), |
| | 7.12-7.21(m, 1H), 6.83 (d, J = 8.4 Hz, 1H), 6.53 (d, J = 8.1 Hz, 1H), 4.85 (s, |
| | 1H), 4.11 (s, 2H), 3.74 (s, 4H), 3.65 (s, 2H), 2.70 (s, 4H), 1.15 (s, 6H) |
| 59 | MS (ESI) calcd. for C₃₂H₃₇N₅O₅, 571.28 m/z, found 572.30 [M + H]⁺ ¹H NMR |
| | (400 MHZ, DMSO-d₆) δ 12.72 (s, 1H), 11.64 (s, 1H), 10.44 (s, 1H), 7.97 (d, J = |
| | 7.8 Hz, 1H), 7.76 (s, 1H), 7.54-7.39 (m, 4H), 7.12 (d, J = 8.2 Hz, 1H), 6.72 |
| | (d, J = 8.4 Hz, 1H), 6.25 (d, J = 8.2 Hz, 1H), 4.95 (s, 1H), 4.09 (s, 2H), 3.50 (s, |
| | 2H), 2.45-2.21 (m, 10H), 2.14 (s, 4H), 1.13 (s, 6H) |
| 60 | MS (ESI) calcd. for C₃₀H₃₃FN₆O₅, 576.25 m/z, found 577.30 [M + H]⁺. ¹H- |
| | NMR (300 MHz, DMSO-d₆) δ (ppm): 12.91 (s, 1H), 10.23 (s, 1H), 7.55-7.64 |
| | (m, 2H), 7.50-7.53 (m, 2H), 7.39-7.40 (m, 1H), 7.17- 7.20 (m, 1H), 6.92- |
| | 6.94 (m, 1H), 6.75-6.78 (m, 1H), 4.85 (s, 1H), 4.15 (s, 2H), 3.34 (s, 2H), 3.18- |
| | 3.47 (m, 8H), 2.78-2.79 (s, 3H), 1.20 (s, 6H). ¹⁹F NMR (282 MHZ, DMSO- |
| | d₆) δ (ppm): −70.08. |
| 61 | MS (ESI) calcd. for C₃₂H₃₇N₅O₅, 571.28 m/z, found 572.30 [M + H]⁺. ¹H NMR |
| | (300 MHz, DMSO-d₆) δ 12.73 (s, 1H), 11.57 (s, 1H), 10.37 (s, 1H), 7.84 (d, J = |
| | 1.6 Hz, 1H), 7.66 (t, J = 2.0 Hz, 1H), 7.60-7.49 (m, 2H), 7.45 (d, J = 8.1 Hz, |
| | 1H), 7.20-7.09 (m, 2H), 6.76 (d, J = 8.4 Hz, 1H), 6.42 (dd, J = 8.2, 0.8 Hz, 1H), |
| | 4.94 (s, 1H), 4.11 (s, 2H), 3.53 (s, 2H), 2.41 (s, 11H), 2.21 (s, 3H), 1.15 (s, 6H) |
| 64 | MS (ESI) calcd. for C₃₀H₃₄N₆O₅, 558.26 m/z, found 559.35 [M + H]⁺. ¹H NMR |
| | (300 MHz, DMSO-d₆) δ (ppm): 10.22 (s, 1H), 8.22-9.00 (m, 1H), 7.53-7.88 |
| | (m, 4H), 6.85-7.52 (m, 3H), 6.69-6.80 (m, 1H), 4.15-4.33 (m, 2H), 3.91- |
| | 4.10 (m, 2H), 2.92-3.50 (m, 8H), 2.82 (s, 3H), 1.16-1.35 (m, 6H) |
| 65 | MS (ESI) calcd. for C₃₀H₃₄N₆O₄, 542.35m/z, found 543.35 [M + H]⁺ ¹H NMR |
| | (400 MHz, DMSO-d₆) δ 12.94 (s, 1H), 11.32 (s, 1H), 10.24 (s, 1H), 8.28 (d, J = |
| | 5.1 Hz, 1H), 7.82 (dd, J = 5.0, 1.3 Hz, 1H), 7.70 (d, J = 1.2 Hz, 1H), 7.61 (d, J = |
| | 8.3 Hz, 3H), 7.26 (d, J = 8.3 Hz, 1H), 7.05-6.86 (m, 1H), 6.72 (dd, J = 8.1, |
| | 0.9 Hz, 1H), 4.10 (s, 2H), 3.80 (d, J = 7.4 Hz, 1H), 3.41 (s, 2H), 3.05 (s, 4H), |
| | 2.79 (s, 4H), 2.42-2.28 (m, 2H), 0.95 (d, J = 6.7 Hz, 6H) |
| 66 | MS (ESI) calcd. for C₃₀H₃₃FN₆O₅, 576.25 m/z, found: 577.25[M + H]⁺. ¹H |
| | NMR (300 MHz, DMSO-d₆) δ (ppm): 12.90 (s, 1H), 10.14 (s, 1H), 8.26-8.28 |
| | (m, 1H), 7.82-7.84 (m, 1H), 7.70 (s, 1H), 7.56 (s, 1H), 7.47-7.49 (m, 1H), |
| | 7.15-7.18 (m, 1H), 6.68-6.75 (m, 2H), 4.91 (s, 1H), 4.18 (s, 2H), 3.43-3.55 |
| | (m, 2H), 2.39-2.59 (m, 8H), 2.29 (s, 3H), 1.20 (s, 6H). 19F NMR (282 MHZ, |
| | DMSO-d6) δd (ppm): −98.13 |
| 67 | MS (ESI) calcd. for C₃₄H₄₁N₇O₅, 627.32 m/z, found 628.30 [M + H]⁺. ¹H NMR |
| | (400 MHZ, DMSO-d₆) δ 12.80 (s, 1H), 11.33 (s, 1H), 10.23 (s, 1H), 8.27 (d, J = |
| | 5.2 Hz, 1H), 7.89-7.70 (m, 1H), 7.68 (s, 1H), 7.60 (t, J = 8.2 Hz, 1H), 7.59- |
| | 7.21 (m, 3H), 7.00 (d, J = 8.7 Hz, 1H), 6.89 (d, J = 8.4 Hz, 1H), 6.71 (d, J = 8.0 |
| | Hz, 1H), 4.29 (s, 1H), 4.19 (s, 2H), 3.89-3.80 (m, 3H), 3.07 (s, 4H), 2.89- |
| | 2.71 (m, 4H), 2.11-2.03 (m, 4H), 2.11- 1.98 (m, 2H), 1.81-1.50 (m, 2H), 1.24 |
| | (s, 6H). |
| 68 | ¹H NMR (400 MHZ, DMSO-d₆) δ 12.91 (br s, 1H), 10.21 (s, 1H), 8.30 (br s, |
| | 1H), 8.21 (d, J = 5.2 Hz, 1H), 7.81 (d, J = 5.2 Hz, 1H), 7.72 (s, 1H), 7.56-7.61 |
| | (m, 2H), 7.48-7.51 (m, 1H), 7.18 (d, J = 8.0 Hz, 1H), 6.90-6.93 (m, 1H), 4.94 |
| | (br s, 1H), 4.20 (s, 2H), 3.51 (s, 2H), 2.21-2.50 (m, 8H), 2.16 (s, 3H), 1.23 (s, |
| | 6H). ¹⁹F NMR (376 MHz, DMSO-d₆) δ −140.44. MS (ESI) calcd. for |
| | C₃₀H_{33F}N₆O₅, 576.25 m/z, found 577.25 [M + H]⁺. |
| 70 | MS (ESI) calcd. for C₃₂H₃₇N₅O₆, 587.27 m/z, found 588.35 [M + H]⁺. ¹H NMR |
| | (400 MHZ, DMSO-d₆) δ 12.73 (s, 1H), 11.33 (s, 1H), 10.23 (s, 1H), 8.39-8.26 |
| | (m, 1H), 7.92-7.75 (m, 1H), 7.67 (t, J = 1.0 Hz, 1H), 7.59 (t, J = 8.4 Hz, 1H), |
| | 7.38 (d, J = 8.8 Hz, 1H), 7.19 (d, J = 2.2 Hz, 1H), 7.09-6.90 (m, 2H), 6.89- |
| | 6.71 (m, 1H), 5.03 (s, 1H), 4.17 (d, J = 8.0 Hz, 3H), 3.81-3.60 (m, 2H), 2.58- |
| | 2.51 (m, 2H), 1.90-1.71 (m, 2H), 1.45-1.29 (m, 3H), 1.21 (s, 6H), 1.08 (s, |
| | 6H). |
| 71 | Mass calcd. for C₃₀H₃₂N₆O₆, 572.24 m/z, found 573.30 [M + H]⁺ ¹H NMR (300 |
| | MHz, DMSO-d₆) δ (ppm): 13.05 (s, 2H), 11.32 (s, 1H), 10.23 (s, 1H), 8.27- |
| | 8.29(m, 1H), 7.82-7.83 (m, 1H), 7.69-7.71 (m, 2H), 7.57-7.62 (m, 2H), 7.28- |
| | 7.31 (m, 1H), 6.87-6.90 (m, 1H), 6.70-6.72 (m, 1H), 4.89 (s, 1H), 4.21 (s, |
| | 2H), 3.35 (s, 4H), 2.50-2.39 (m, 7H), 1.22 (s, 6H) |
| 73 | MS (ESI) calcd. for C₂₉H₃₃N₅O₅S, 563.22 m/z, found 564.25 [M + H]⁺. ¹H NMR |
| | (300 MHz, DMSO-d₆) δ 12.59 (s, 1H), 10.38 (s, 1H), 8.19 (s, 1H), 7.62-7.48 |
| | (m, 3H), 7.43 (d, J = 8.1 Hz, 1H), 7.14 (d, J = 8.3 Hz, 1H), 6.84-6.74 (m, |
| | 2H), 6.63 (d, J = 8.2 Hz, 1H), 4.92 (s, 1H), 4.10 (s, 2H), 3.51 (s, 2H), 2.49- |
| | 2.29 (m, 7H), 2.15 (s, 3H), 1.20 (s, 6H). |
| 75 | Mass calcd. for C₂₅H₂₄N₄O₅, 602.29 m/z, found 603.35[M + H]⁺. ¹H NMR (300 |
| | MHz, DMSO-d₆) δ (ppm): 12.96 (s, 1H), 11.31 (s, 1H), 10.23 (s, 1H), 8.27- |
| | 8.28 (m, 1H), 7.81-7.83 (m, 1H), 7.69 (s, 1H), 7.53-7.63 (m, 3H), 7.20-7.30 |
| | (m, 1H), 6.88-6.90 (m, 1H), 6.70-6.75 (m, 1H), 4.20 (s, 2H), 3.64 (s, 2H), |
| | 3.28-3.33 (m, 7H), 2.98-3.13 (m, 4H), 2.41-2.58 (m, 4H), 1.23 (s, 6H) |
| 76 | MS (ESI) calcd. for C₂₉H₃₁N₅O₆, 545.227 m/z, found 546.15 [M + H]⁺. ¹H NMR |
| | (300 MHz, DMSO-d₆) δ (ppm): 12.91 (s, 1H), 11.32 (s, 1H), 10.22 (s, 1H), |
| | 8.20-8.30 (m, 1H), 7.75-7.85 (m, 1H), 7.65-7.75 (m, 1H), 7.55-7.62 (m, |
| | 2H), 7.45-7.55 (m, 1H), 7.15-7.28 (m, 1H), 6.85-6.95 (m, 1H), 6.69-6.77 |
| | (m, 1H), 4.92 (s, 1H), 4.19 (s, 2H), 3.20-3.72 (m, 6H), 2.25-2.65 (m, 4H), |
| | 1.21 (s, 6H). |
| 77 | MS (ESI) calcd. for C₃₀H₃₃FN₆O₇: 576.25. Found: 577.25 [M + H]⁺. ¹H NMR |
| | (300 MHz, DMSO-d₆) δ (ppm): 12.94 (s, 1H), 11.31 (s, 1H), 10.19 (s, 1H), |
| | 8.24-8.26 (m, 1H), 7.79-7.81 (m, 1H), 7.51-7.67 (m, 4H), 7.20-7.23 (m, |
| | 1H), 6.74-6.78 (m, 1H), 4.19 (s, 2H), 3.65-3.71 (m, 6H), 2.98-3.11 (m, 4H), |
| | 2.77 (s, 3H), 1.22 (s, 6H). 19F NMR (282 MHZ, DMSO-d6) δd (ppm): −139.61. |
| 79 | MS (ESI) calcd. for C₃₁H₃₆N₆O₅: 572.27. Found: 573.25 [M + H]⁺. ¹H-NMR |
| | (300 MHz, DMSO-d₆) δ (ppm): 12.85 (s, 1H), 10.23 (s, 1H), 7.65 (s, 1H), 7.53- |
| | 7.59 (m, 2H), 7.44-7.47 (m, 2H), 7.13-7.16 (m, 1H), 6.82-6.86 (m, 1H), |
| | 6.63-6.66 (m, 1H), 4.98 (s, 1H), 4.14 (s, 2H), 3.51 (s, 2H), 2.35-2.49 (m, |
| | 11H), 2.15 (s, 3H), 1.20 (s, 6H). |
| 80 | Mass calcd. for C₃₃H₃₉N₅O₆, 601.29 m/z, found 602.35 [M + H]⁺. ¹H NMR |
| | (300 MHz, DMSO-d₆) δ (ppm): 8.69 (s, 1H), 8.28 (s, 1H), 8.11 (s, 1H), 7.675 |
| | (s, 1H), 7.56-7.59 (m, 1H), 7.45-7.50 (m, 1H), 7.36-7.39 (m, 1H), 6.90- |
| | 6.97 (m, 2H), 4.30-4.36 (m, 4H), 3.53-3.57 (m, 2H), 2.95-3.03 (m, 2H), |
| | 2.01-2.05 (m, 2H), 1.47-1.66 (m, 3H), 1.36 (s, 6H), 1.88(s, 6H). |
| 82 | MS (ESI) calcd. for C₃₀H₃₃FN₆O₅, 576.25 m/z, found 577.30 [M + H]⁺. ¹H-NMR |
| | (300 MHz, DMSO-d₆) δ (ppm): ¹H-NMR (300 MHZ, DMSO-d⁶) δ (ppm): |
| | 12.85 (s, 1H), 11.27 (s, 1H), 10.24 (s, 1H), 7.91-7.93 (m, 1H), 7.61-7.68 (m, |
| | 1H), 7.54-7.60 (m, 3H), 7.26-7.28 (m, 1H), 6.90-6.93 (m, 1H), 6.73-6.76 |
| | (m, 1H), 4.19 (s, 2H), 3.94 (s, 2H), 3.08-3.47 (m, 6H), 2.80 (s, 3H), 2.78- |
| | 2.79 (m, 2H), 1.20 (s, 6H) |
| 86 | MS (ESI) calcd. for C30H33CIN6O5, 592.22 m/z, found 593.35 [M + H]+. 1H |
| | NMR (300 MHz, DMSO-d₆) δ 12.91 (s, 1H), 10.25 (s, 1H), 7.77 (d, J = 1.0 |
| | Hz, 1H), 7.68-7.54 (m, 3H), 7.50 (d, J = 8.0 Hz, 1H), 7.19 (d, J = 8.1 Hz, 1H), |
| | 6.94 (d, J = 8.3 Hz, 1H), 6.78 (d, J = 7.9 Hz, 1H), 4.89 (s, 1H), 4.17 (s, 2H), |
| | 3.53 (s, 2H), 2.36 (s, 9H), 2.16 (s, 3H), 1.21 (s, 6H) |
| 92 | MS (ESI) calcd. for C₃₀H₃₄N₆O₄, 542.26 m/z, found 543.25 [M + H]⁺. ¹H NMR |
| | (300 MHz, DMSO-d⁶) δ (ppm): 12.89 (s, 1H), 10.16 (s, 1H), 8.22-8.24 (m, |
| | 1H), 7.89-7.91 (m, 1H), 7.75-7.80 (m, 3H), 7.49-7.56 (m, 1H), 7.44-7.46 |
| | (m, 2H), 7.31-7.33 (m, 1H), 7.15-7.18 (m, 1H), 4.92 (s, 1H), 4.18 (s, 2H), |
| | 3.55 (s, 2H), 2.41-2.49 (m, 8H), 2.22 (s, 3H), 1.19 (s, 6H). |
| 93 | MS (ESI) calcd. for C₃₀H₃₄N₆O₅, 558.26 m/z, found 559.35 [M + H]⁺. ¹H NMR |
| | (300 MHz, DMSO-d₆) δ (ppm): δ 12.78 (s, 1H), 11.37 (s, 1H), 10.24 (s, 1H), |
| | 8.02 (s, 2H), 7.57 (d, J = 8.3 Hz, 1H), 7.52 (d, J = 9.1 Hz, 1H), 7.42 (d, J = 7.6 |
| | Hz, 1H), 7.27 (s, 1H), 7.12 (d, J = 8.4 Hz, 1H), 6.83 (d, J = 8.3 Hz, 1H), 6.73 |
| | (d, J = 8.2 Hz, 1H), 4.99 (s, 1H), 4.07 (s, 2H), 3.53 (s, 2H), 2.46-2.29 (m, |
| | 7H), 2.24 (s, 4H), 1.11 (s, 6H) |
| 98 | MS (ESI) calcd. for C₃₂H₃₇N₇O₅, 599.29 m/z, found 600.35 [M + H]⁺. ¹H NMR |
| | (400 MHZ, DMSO-d6) δ (ppm): 12.98 (s, 1H), 11.12 (s, 1H), 10.21 (s, 1H), |
| | 8.26-8.29 (m, 2H), 7.84-7.85 (m, 1H), 7.71-7.73 (m, 2H), 7.62-7.69 (m, |
| | 1H), 7.53-7.55 (m, 1H), 7.23-7.25 (m, 1H), 6.97-6.99 (m, 1H), 4.19 (s, 2H), |
| | 3.48-3.82 (m, 4H), 3.05-3.42 (s, 5H), 2.78 (s, 3H), 2.18 (s, 3H), 1.04-1.22 |
| | (m, 6H) |
| 105 | MS (ESI) calcd. for C₃₁H₃₅N₅O₅, 557.264 m/z, found 558.30 [M + H]⁺. 1H |
| | NMR (300 MHz, DMSO-d6) δ (ppm): 12.75 (s, 1H), 10.35 (s, 1H), 8.20-8.30 |
| | (m, 2H), 7.41-7.56 (m, 3H), 7.10-7.21 (m, 3H), 6.72-6.78 (m, 1H), 6.38- |
| | 6.43 (m, 1H), 5.05 (s, 1H), 4.19 (s, 2H), 3.52 (s, 2H), 2.23-2.43 (m, 8H), 2.15 |
| | (s, 3H), 1.22 (s, 6H) |
| 107 | MS (ESI) calcd. for C₃₃H₃₅N₅O_{5S}, 613.24 m/z, found 614.20 [M + H]⁺. ¹H NMR |
| | (400 MHZ, DMSO-d₆) δ 12.75 (s, 1H), 11.51 (s, 1H), 10.40 (s, 1H), 8.54 (d, J = |
| | 1.2 Hz, 1H), 7.90 (d, J = 5.3 Hz, 1H), 7.76 (d, J = 1.2 Hz, 1H), 7.71 (d, J = 5.4 |
| | Hz, 1H), 7.57 (t, J = 8.3 Hz, 1H), 7.52 (d, J = 1.4 Hz, 1H), 7.44 (d, J = 8.1 Hz, |
| | 1H), 7.19-7.09 (m, 1H), 6.86 (d, J = 8.4 Hz, 1H), 6.57 (d, J = 8.2 Hz, 1H), |
| | 4.89 (s, 1H), 4.09 (s, 2H), 3.51 (s, 2H), 2.45-2.16 (m, 7H), 2.15 (s, 3H), 1.15- |
| | 1.01 (s, 6H). |
| 108 | MS (ESI) calcd. for C₂₉H₃₃N₇O₅, 559.25 m/z, found 560.25 [M + H]⁺. ¹H NMR |
| | (400 MHZ, DMSO-d₆) δ 12.86 (s, 1H), 11.33 (s, 1H), 10.26 (s, 1H), 9.16 (s, |
| | 1H), 8.70 (s, 1H), 7.61 (t, J = 8.3 Hz, 2H), 7.45 (d, J = 8.1 Hz, 1H), 7.20-7.14 |
| | (m, 1H), 6.99-6.93 (m, 1H), 6.91-6.76 (m, 1H), 5.01-4.85 (m, 1H), 4.11 (s, |
| | 2H), 3.60 (s, 2H), 3.46-3.35 (m, 1H), 2.83 (s, 4H), 2.69-2.52 (m, 3H), 2.49- |
| | 2.01 (m, 3H), 1.15 (s, 6H) |
| 109 | MS (ESI) calcd. for C₃₂H₃₄N₆O₅, 582.66 m/z, found 583.30 [M + H]⁺. ¹H NMR |
| | (300 MHz, DMSO-d₆) δ 12.77 (s, 1H), 11.43 (s, 1H), 10.31 (s, 1H), 7.98 (s, |
| | 2H), 7.70-7.56 (m, 2H), 7.51 (s, 1H), 7.42 (d, J = 8.1 Hz, 1H), 7.20-7.09 (m, |
| | 1H), 6.99-6.90 (m, 1H), 6.74-6.66 (m, 1H), 4.76 (s, 1H), 3.99 (s, 2H), 3.49 |
| | (s, 2H), 2.35-2.25 (m, 7H), 2.14 (s, 3H), 1.05 (s, 6H). |
| 110 | MS (ESI) calcd. for C₃₁H_{34F}N₅O₅, 575.25 m/z, found 576.30 [M + H]⁺. ¹H NMR |
| | (300 MHz, DMSO-d₆) δ 12.75 (s, 1H), 10.44 (s, 1H), 8.19-8.09 (m, 1H), 8.03- |
| | 7.96 (m, 1H), 7.59-7.48 (m, 3H), 7.45 (d, J = 8.1 Hz, 1H), 7.14 (d, J = 8.2, 1.4 |
| | Hz, 1H), 6.79 (d, J = 8.4 Hz, 1H), 6.41 (d, J = 8.2 Hz, 1H), 4.90 (s, 1H), 4.10 |
| | (s, 2H), 3.50 (d, J = 7.4 Hz, 7H), 2.34 (d, J = 13.1 Hz, 4H), 2.17 (s, 3H), 1.15 |
| | (s, 6H). ¹⁹F NMR (282 MHZ, DMSO-d₆) δ −128.54. |
| 111 | MS (ESI) calcd. for C₃₄H₃₈N₆O₆, 626.29 m/z, found 627.35 [M + H]⁺. ¹H NMR |
| | (400 MHZ, DMSO-d₆) δ 12.73 (s, 1H), 11.56 (s, 1H), 10.43 (s, 1H), 10.36 (s, |
| | 1H), 8.25-8.15 (m, 1H), 7.81 (t, J = 2.2 Hz, 1H), 7.61-7.50 (m, 3H), 7.45 (d, |
| | J = 8.1 Hz, 1H), 7.14 (dd, J = 8.2, 1.4 Hz, 1H), 6.79 (d, J = 8.4 Hz, 1H), 6.53- |
| | 6.40 (m, 2H), 6.29 (dd, J = 17.0, 2.0 Hz, 1H), 5.80 (dd, J = 10.0, 2.0 Hz, 1H), |
| | 4.85 (s, 1H), 4.11 (s, 2H), 3.52 (s, 2H), 2.52-2.31 (m, 7H), 2.16 (s, 3H), 1.15 (s, |
| | 6H) |

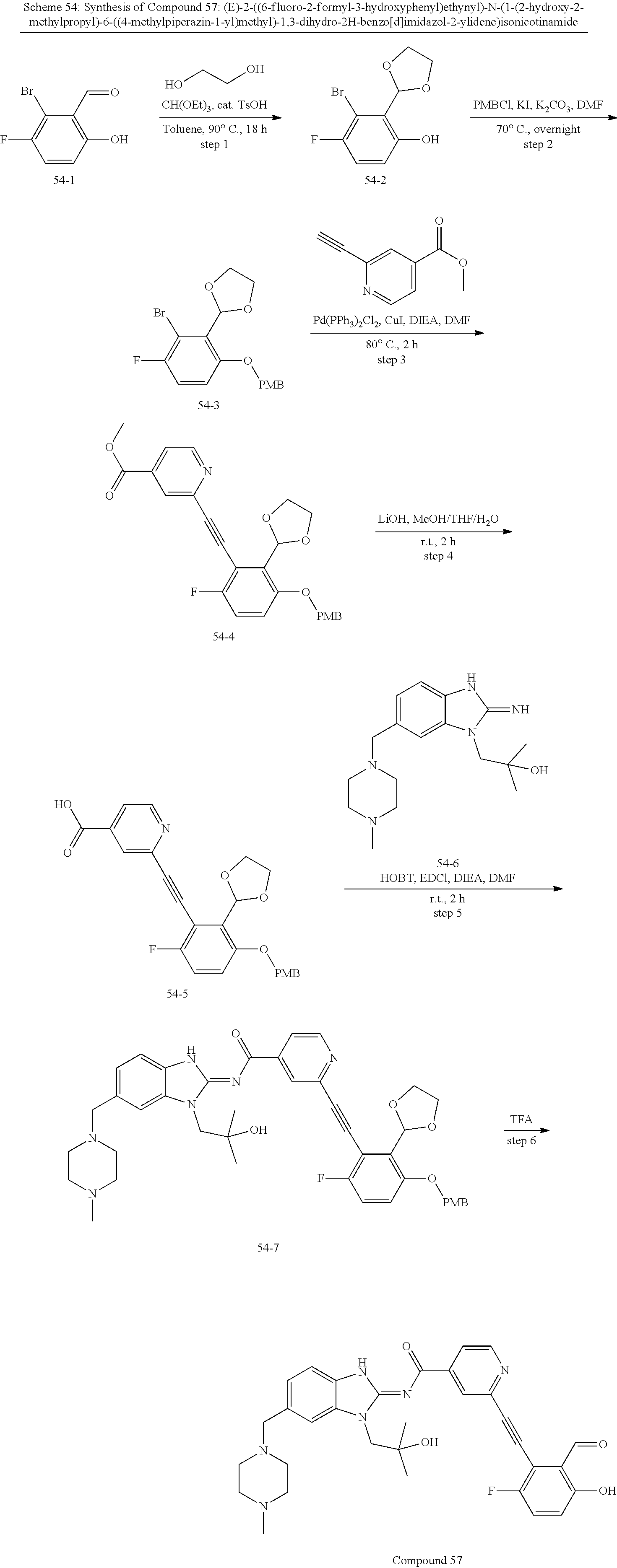

### Step 1: Synthesis of 3-bromo-2-(1,3-dioxolan-2-yl)-4-fluorophenol

To a 250 ml round-bottomed flask equipped with a stirring bar were added methyl 2-bromo-3-fluoro-6-hydroxybenzaldehyde (2 g, 9.132 mmol, 1 equiv) and Toluene (15 mL, 140.980 mmol, 15.44 equiv), followed by addition of ethylene glycol (2.83 g, 45.660 mmol, 5 equiv), TsOH (0.16 g, 0.913 mmol, 0.1 equiv), ethylene glycol (2.83 g, 45.660 mmol, 5 equiv).

The resulting solution was stirred at 90° C. for 2 h. The resulting mixture was extracted with EA (3×150 mL), dried over anhydrous Na₂SO₄. After filtration, the filtrate was concentrated under reduced pressure. The residue was purified by silica gel column chromatography, eluted with PE/EA (6%) to afford 3-bromo-2-(1,3-dioxolan-2-yl)-4-fluorophenol (900 mg, 35.67%) as a white solid. MS (ESI) calcd. for C₉H₈BrFO₃, 262.00 m/z, found 263.00 [M+H]⁺.

### Step 2: Synthesis of 2-(2-bromo-3-fluoro-6-((4-methoxybenzyl)oxy)phenyl)-1,3-dioxolane

Into a 25° C. were added 3-bromo-2-(1,3-dioxolan-2-yl)-4-fluorophenol (1.8 g, 6.842 mmol, 1 equiv) 3-bromo-2-(1,3-dioxolan-2-yl)-4-fluorophenol (1.8 g, 6.842 mmol, 1 equiv) PMBCl (1.61 g, 10.263 mmol, 1.5 equiv) and K₂CO₃ (1.89 g, 13.684 mmol, 2 equiv) at 100 mL round-bottom flask. Desired product was detected by LCMS. The reaction was quenched by the addition of H₂O (100 mL) at 25° C. The resulting mixture was extracted with EA (3 * 150 mL).

The combined organic layers. Dried over anhydrous Na₂SO₄. After filtration, the filtrate was concentrated under reduced pressure. The residue was purified by silica gel column chromatography, eluted with PE/EA (6%) to afford as a white solid. to afford 2-{2-bromo-3-fluoro-6-[(4-methoxyphenyl)methoxy]phenyl}-1,3-dioxolane (1.1 g, 40.27%) as a white solid.

MS (ESI) calcd. for C₁₇H₁₆BrFO₄, 382.02 m/z, found 383.02 [M+H]⁺.

### Step 3: Synthesis of ethyl 2-((2-(1,3-dioxolan-2-yl)-6-fluoro-3-((4-methoxybenzyl)oxy)phenyl)ethynyl)isonicotinate

To a 50 ml round-bottomed flask equipped with a stirring bar were added methyl 2-{2-bromo-3-fluoro-6-[(4-methoxyphenyl)methoxy]phenyl}-1,3-dioxolane (300 mg, 0.783 mmol, 1 equiv) and DMF (4 mL, 51.686 mmol, 66.02 equiv), followed by addition of methyl 2-ethynylpyridine-4-carboxylate (138.78 mg, 0.861 mmol, 1.1 equiv), CuI (7.45 mg, 0.039 mmol, 0.05 equiv), Pd(PPh₃)₂C₁₂ (27.47 mg, 0.039 mmol, 0.05 equiv) and DIEA (202.36 mg, 1.566 mmol, 2 equiv). The resulting solution was stirred at 80° C. for 2 h under the N2. The resulting mixture was extracted with EA (3 * 10 mL), dried over anhydrous Na₂SO₄. After filtration, the filtrate was concentrated under reduced pressure. The residue obtained was purified by reverse phase column (65% ACN) to afford the methyl 2-{2-[2-(1,3-dioxolan-2-yl)-6-fluoro-3-[(4-methoxyphenyl)methoxy] phenyl]ethynyl}pyridine-4-carboxylate (220 mg, 60.64%). MS (ESI) calcd. for C₂₆H₂₂FNO₆, 463.46 m/z, found 464.35 [M+H]⁺.

### Step 4: Synthesis of 2-((2-(1,3-dioxolan-2-yl)-6-fluoro-3-((4-methoxybenzyl)oxy)phenyl)ethynyl)isonicotinic acid

To a 25 ml round-bottomed flask equipped with a stirring bar was added methyl 2-{2-[2-(1,3-dioxolan-2-yl)-6-fluoro-3-[(4-methoxyphenyl)methoxy]phenyl]ethynyl}pyridine-4-carboxylate (220 mg, 0.475 mmol, 1 equiv), THF (2 mL, 24.686 mmol, 52.00 equiv), followed by addition of LiOH (34.11 mg, 1.424 mmol, 3.00 equiv) in H₂O (1 mL, 55.509 mmol, 116.94 equiv). The resulting solution was stirred at rt for 0.5 h. The mixture was acidified neutralized to pH 7 with HCl (2M). The solvents was evaporated to afford 2-{2-[2-(1,3-dioxolan-2-yl)-6-fluoro-3-[(4-methoxyphenyl)methoxy]phenyl]ethynyl}pyridine-4-carboxylic acid (110 mg, 48.98%). MS (ESI) calcd. for C₂₅H₂₀FNO₆, 449.13 m/z, found 450.13 [M+H]⁺.

### Step 5: Synthesis of (E)-2-((2-(1,3-dioxolan-2-yl)-6-fluoro-3-((4-methoxybenzyl)oxy)phenyl)ethynyl)-N-(1-(2-hydroxy-2-methylpropyl)-6-((4-methylpiperazin-1-yl)methyl)-1,3-dihydro-2H-benzo[d]imidazol-2-ylidene)isonicotinamide

A solution of 2-{2-[2-(1,3-dioxolan-2-yl)-6-fluoro-3-[(4-methoxyphenyl)methoxy]phenyl]ethynyl}pyridine-4-carboxylic acid (100 mg, 0.223 mmol, 1 equiv), 1-{2-imino-6-[(4-methylpiperazin-1-yl)methyl]-3H-1,3-benzodiazol-1-yl}-2-methylpropan-2-ol (77.69 mg, 0.245 mmol, 1.1 equiv), HATU (126.90 mg, 0.335 mmol, 1.5 equiv) in DMF (2.5 mL, 32.304 mmol, 145.18 equiv), followed by the addition of DIEA (57.52 mg, 0.446 mmol, 2 equiv). The resulting mixture was stirred for 10 min at rt. The crude product (100 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 10% B to 30% B in 10 min, 30% B; Wave Length: 254/220 nm; RT1(min): 9.6; Number Of Runs: 0) to afford 2-{2-[2-(1,3-dioxolan-2-yl)-6-fluoro-3-[(4-methoxyphenyl)methoxy]phenyl]ethynyl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (35 mg, 16.80%) as a white solid. MS (ESI) calcd. for C₄₂H₄₅FN₆O₆, 748.34 m/z, found 749.34 [M+H]⁺.

### Step 6: Synthesis of (E)-2-((6-fluoro-2-formyl-3-hydroxyphenyl)ethynyl)-N-(1-(2-hydroxy-2-methylpropyl)-6-((4-methylpiperazin-1-yl)methyl)-1,3-dihydro-2H-benzo[d]imidazol-2-ylidene)isonicotinamide

To a stirred solution of 2-{2-[2-(1,3-dioxolan-2-yl)-6-fluoro-3-[(4-methoxyphenyl)methoxy]phenyl]ethynyl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (45 mg, 0.060 mmol, 1 equiv) in trifluoroacetaldehyde (3 mL) at room temperature. The resulting mixture was stirred for 10 min at room temperature. The resulting mixture was concentrated under reduced pressure. The crude product (45 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 13% B to 37% B in 8 min, 37% B; Wave Length: 254/220 nm; RT1(min): 8; Number Of Runs: 0) to afford 2-[2-(6-fluoro-2-formyl-3-hydroxyphenyl)ethynyl]-N-[(2E)-1-(2-hydroxy-2-methylpropyl)-6-[(4-methylpiperazin-1-yl)methyl]-3H-1,3-benzodiazol-2-ylidene]pyridine-4-carboxamide (6.5 mg, 18.45%) as a yellow solid. MS (ESI) calcd. for C₃₂H₃₃FN₆04, 584.25 m/z, found 585.39 [M+H]⁺. ¹H NMR (300 MHz, DMSO-d₆) δ (ppm): 12.90 (s, 1H), 10.47 (s, 1H), 8.79-8.81 (m, 1H), 8.36 (s, 1H), 8.10-8.14 (m, 1H), 8.17-8.18 (m, 2H), 7.58-7.64 (m, 1H), 7.49-7.53 (m, 2H), 4.90 (s, 1H), 4.24-4.25 (m, 2H), 3.56-3.66 (m, 2H), 2.26-2.51 (m, 8H), 2.26-2.50 (m, 3H), 1.17-1.26 (m, 6H). ¹⁹F NMR (282 MHz, DMSO-d₆) δ (ppm): -119.95.

The following compounds were synthesized according to the preceding method or analogous methods thereto:

| Cmpd No. | Characterization Data |
|---|---|
| 62 | [0678] MS (ESI) calcd. for C₃₂H₃₄N₆O₄: 566.26 m/z, found: 567.25 [M + H]⁺. |
| | ¹H NMR (300 MHz, DMSO-d₆) δ (ppm): 11.88 (s, 1H), 10.54 (s, 1H), 8.78- |
| | 8.80 (m, 1H), 8.34 (s, 1H), 8.08-8.11 (m, 1H), 7.49-7.66 (m, 3H), 7.29-7.31 |
| | (m, 1H), 7.10-7.20 (m, 2H), 4.92 (s, 1H), 4.25 (s, 1H), 4.24 (s, 2H), 3.44- |
| | 3.51 (m, 2H), 2.33-2.73 (m, 8H), 2.15 (s, 3H), 1.16-1.26 (m, 6H). |
| 69 | MS (ESI) calcd. for C₃₃H₃₇N₇O₅S, 643.258 m/z, found 644.30 [M + H]⁺. 1H |
| | NMR (300 MHz, DMSO-d6) δ (ppm): 12.84-12.92 (m, 1H), 10.58 (s, 1H), |
| | 8.75-8.83 (m, 1H), 8.32-8.41 (m, 1H), 8.07-8.21 (m, 1H), 7.48-7.81 (m, |
| | 5H), 7.18-7.22 (m, 1H), 4.94 (s, 1H), 4.24 (s, 2H), 3.51-3.60 (m, 3H), 3.21 |
| | (s, 2H), 2.28-2.49 (m, 8H), 2.20(s, 3H), 1.26(s, 6H) |
| 72 | MS (ESI) calcd. for C₃₂H₃₃FN₆O₄, 584.25 m/z, found 585.30 [M + H]⁺. ¹H NMR |
| | (300 MHz, DMSO-d6) δ (ppm): 10.47 (s, 1H), 8.73-8.80 (m, 1H), 8.31 (s, |
| | 1H), 8.02-8.10 (m, 1H), 7.59 (s, 1H), 7.40-7.54 (m, 2H), 7.13-7.22 (m, 1H), |
| | 7.01-7.11 (m, 1H), 4.25 (s, 2H), 3.60 (s, 2H), 2.40-2.80 (m, 8H), 2.36 (s, |
| | 3H), 1.26 (s, 6H) |
| 78 | MS (ESI) calcd. for C₃₁H₃₁N₅O₅: 553.23 m/z, found: 554.25 [M + H]⁺. ¹H-NMR |
| | (300 MHz, DMSO-d6) δ (ppm): 12.88 (s, 1H), 11.34 (s, 1H), 10.53 (s, 1H), |
| | 8.78-8.80 (m, 1H), 8.34 (s, 1H), 8.09-8.11 (m, 1H), 7.60-7.66 (m, 2H), 7.50- |
| | 7.52 (m, 1H), 7.29-7.31 (m, 2H), 7.19-7.21 (m, 1H), 7.10-7.13 (m, 1H), |
| | 4.92 (s, 1H), 4.25 (s, 1H), 3.55-3.58 (m, 6H), 2.28-2.37 (m, 4H), 1.20-1.31 |
| | (m, 6H). |
| 84 | MS (ESI) calcd. for C₃₄H₃₇N₇O₅, 623.29 m/z, found 624.35 [M + H]⁺. ¹H NMR |
| | (300 MHz, DMSO-d6) δ (ppm): 12.85 (s, 1H), 10.85 (s, 1H), 10.60 (s, 1H), |
| | 8.95-8.95 (m, 1H), 8.24-8.40 (m, 2H), 8.13 (s, 1H), 7.75-7.85 (m, 1H), 7.39- |
| | 7.59 (m, 3H), 7.19-7.25 (m, 1H), 4.91 (s, 1H), 4.22 (s, 2H), 3.74 (s, 3H), |
| | 3.40-3.45 (m, 2H), 2.20-2.45 (m, 8H), 2.13 (s, 8H), 1.23 (m, 6H) |
| 87 | MS (ESI) calcd. for C₃₇H₄₁N₇O₆, 633.31 m/z, found 634.35 [M + H]⁺. ¹H NMR |
| | (300 MHz, DMSO-d₆) δ (ppm): 12.88 (s, 1H), 11.39 (s, 1H), 10.57 (s, 1H), |
| | 8.79-8.90 (m, 1H), 8.41-8.55 (m, 1H), 8.31-8.40 (m, 1H), 8.09-8.15 (m, |
| | 1H), 7.68-7.83 (m, 1H), 77.45-7.64 (m, 3H), 7.17-7.25 (m, 1H), 4.90 (s, |
| | 1H), 4.23 (s, 2H), 3.55 (s, 2H), 2.25-2.45 (m, 8H), 2.16 (s, 3H), 1.80-1.90 |
| | (m, 1H), 1.24 (s, 6H), 0.85-0.95 (m, 4H). |
| 89 | MS (ESI) calcd. for C₃₂H₃₃FN₆O₄, 584.25 m/z, found 585.25 [M + H]⁺. 1H |
| | NMR (300 MHz, DMSO-d6) δ (ppm): 10.17-10.14 (m, 1H), 9.93 (s, 1H), 8.78- |
| | 8.80 (m, 1H), 8.10-8.18 (m, 2H), 7.44-7.49 (m, 2H), 6.67-7.16 (m, 2H), |
| | 4.90 (s, 1H), 4.24-4.34 (m, 2H), 3.33-3.53 (m, 2H), 2.31-2.34 (m, 8H), 2.15 |
| | (s, 3H), 1.29 (s, 6H). 19F NMR (282 MHz, DMSO-d₆) δ (ppm): −102.68. |
| 90 | MS (ESI) calcd. for C₃₄H₃₇N₇O₄, 607.29 m/z, found 608.35 [M + H]⁺. ¹H NMR |
| | (300 MHz, DMSO-d₆) δ (ppm): 12.84 (s, 1H), 11.09 (s, 1H), 10.56 (s, 1H), |
| | 8.78-8.88 (m, 1H), 8.38-8.50 (m, 1H), 8.30 (s, 1H), 8.07-8.17 (m, 1H), 7.68- |
| | 7.85 (m, 1H), 7.43-7.62 (m, 3H), 7.13-7.26 (m, 1H), 4.24 (s, 2H), 3.51- |
| | 3.55 (m, 2H), 2.28-2.45 (m, 8H), 2.19 (s, 6H), 1.24 (s, 6H). |
| 97 | MS (ESI) calcd. for C₃₅H₃₉N₇O₄, 621.31 m/z, found 622.31 [M + H]⁺. ¹H NMR |
| | (300 MHz, DMSO-d⁶) δ (ppm): 12.85 (s, 1H), 11.10 (s, 1H), 10.56 (s, 1H), |
| | 8.42-8.45 (m, 1H), 8.14-8.22 (m, 1H), 7.94 (s, 1H), 7.71-7.76 (m, 1H), 7.48- |
| | 7.57 (m, 3H), 7.16-7.18 (m, 1H), 4.93 (s, 1H), 4.23 (s, 2H), 3.52 (s, 2H), 2.60 |
| | (s, 3H), 2.37-2.50 (m, 8H), 2.19 (s, 3H), 2.15 (s, 3H), 1.25 (s, 6H) |
| 99 | MS (ESI) calcd. for C₃₂H₃₅N₇O₃, 565.280 m/z, found 566.30 [M + H]⁺. ¹H NMR |
| | (300 MHz, DMSO-d6) δ (ppm): 12.88 (s, 1H), 10.54 (s, 1H), 8.71-8.85 (m, |
| | 1H), 8.26 (s, 1H), 8.05-8.15 (m, 1H), 7.45-7.70 (m, 4H), 77.32-7.45 (m, |
| | 1H), 7.13-7.23 (m, 1H), 6.85-7.00 (m, 2H), 4.90 (s, 1H), 4.23 (s, 2H), 3.55 |
| | (s, 2H), 2.25-2.45 (m, 8H), 2.16 (s, 3H), 1.24 (s, 6H) |
| 100 | MS (ESI) calcd. for C31H33N7O4, 567.26 m/z, found: 568.25[M + H]⁺. ¹H |
| | NMR (300 MHz, DMSO-d⁶) δ (ppm): 12.90 (s, 1H), 11.37 (s, 1H), 10.53 (s, |
| | 1H), 8.79-8.81 (m, 1H), 8.69-8.79 (m, 1H), 8.49-8.59 (m, 1H), 8.36-8.45 |
| | (m, 1H), 8.11-8.35 (m, 1H), 7.53-7.64 (m, 2H), 7.21-7.27 (m, 1H), 4.10- |
| | 4.25 (m, 2H), 3.78-3.94 (m, 2H), 3.56-3.66 (m, 4H), 2.85-3.19 (m, 4H), |
| | 2.75-2.90 (m, 3H), 1.15-1.28 (m, 6H) |
| 101 | MS (ESI) calcd. for C34H₃₄F₃N₇O4, 661.26 m/z, found: 662.30[M + H]⁺. 1H |
| | NMR (300 MHz, DMSO-d6) δ (ppm): 12.94 (s, 1H), 12.22 (s, 1H), 10.59 (s, |
| | 1H), 8.81-8.86 (m, 1H), 8.36 (s, 1H), 8.20-8.30 (m, 1H), 8.10-8.18 (m, 1H), |
| | 7.83-7.90 (m, 1H), 7.73-7.79 (m, 1H), 7.62-7.70 (m, 1H), 7.50-7.56 (m, |
| | 1H), 7.20-7.30 (m, 1H), 4.24 (s, 2H), 3.80 (s, 2H), 2.90-3.30 (s, 8H), 2.80 (s, |
| | 3H), 1.27 (s, 6H). |

Scheme 55: Synthesis of Compound 63: (E)-2-(2-formyl-3-hydroxyphenoxy)-N-(1-(2-hydroxy-2-methylpropyl)-6-((3-(2-hydroxypropan-2-yl)azetidin-1-yl)methyl)-1,3-dihydro-2H-benzo[d]imidazol-2-ylidene)isonicotinamide

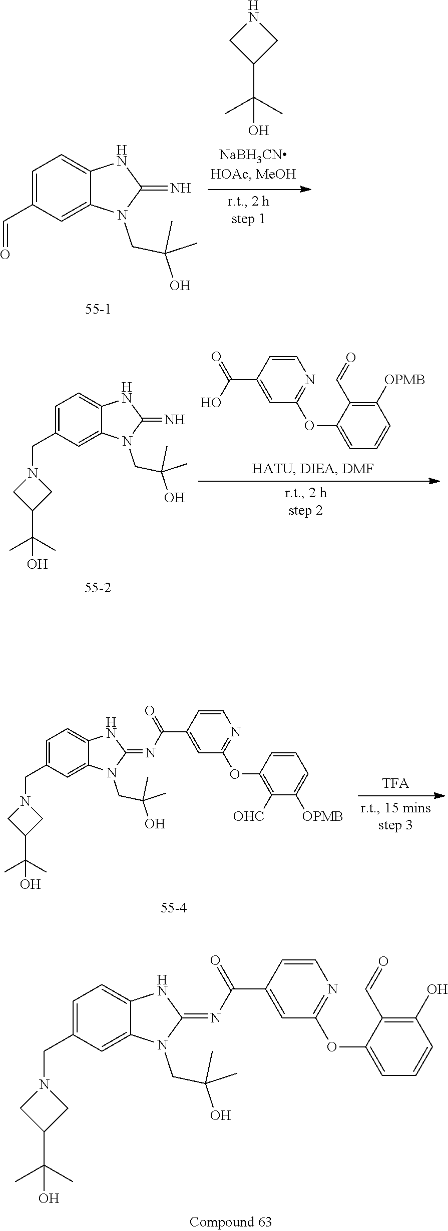

### Step 1: Synthesis of 1-(6-{[3-(2-hydroxypropan-2-yl)azetidin-1-yl]methyl}-2-imino-3H-1,3-benzodiazol-1-yl)-2-methylpropan-2-ol

3-(2-hydroxy-2-methylpropyl)-2-imino-1H-1,3-benzodiazole-S-carbaldehyde(500 mg, 2.143 mmol) and 10 mL MeOH, followed by addition of 2-(azetidin-3-yl)propan-2-ol(370.3lmg, 3.214 mmol), NaBH3CN (199 mg), HOAc (257.5 mg). The resulting solution was stirred at rt for 2 h. The residue was concentrated to dryness in vacuo to give a nearly yellow viscous oil. The crude product was then purified by Prep-HPLC with the following conditions: Column: X Bridge Shield RP¹⁸ OBD Column, 30*150 mm, 5 μm; Mobile Phase A: Water(10 ml/L NH₄HCO₃), Mobile Phase B: ACN; Flow rate: 60 mL/min; Gradient: 11% B to 30% B in 8 min, 30% B; Wave Length: 254/220 nm; RT1(min): 8; Number Of Runs: 0. After lyophilization, it was afforded 1-(6-{[3-(2-hydroxypropan-2-yl)azetidin-1-yl]methyl}-2-imino-3H-1,3-benzodiazol-1-yl)-2-methylpropan-2-ol as a yellow solid (220 mg, 29.87% yield).mass calcd. for C18H₂₈N₄O_{2-, 332.22} found: 333.22 [M+H].

### Step 2: Synthesis of methyl2-{2-formyl-3-[(4-methoxyphenyl)methoxy]phenoxy}-N-[(2E)-3-(2-hydroxy-2-methylpropyl)-1H-imidazo[4,5-b]pyridin-2-ylidene]pyridine-4-carboxamide

1-(6-{[3-(2-hydroxypropan-2-yl)azetidin-1-yl]methyl}-2-imino-3H-1,3-benzodiazol-1-yl)-2-methylpropan-2-ol (130 mg, 0.391 mmol) and 3 mL DMF, followed by addition of 2-{2-formyl-3-[(4-methoxyphenyl)methoxy]phenoxy}pyridine-4-carboxylic acid(163.18 mg, 0.43 mmol), DIEA (100 mg, 4.482 mmol) and HATU (109 mg, 0.319 mmol). The resulting solution was stirred at rt for 2 h. The residue was concentrated to dryness in vacuo to give a nearly yellow viscous oil. The crude product was then purified by Prep-HPLC with the following conditions: Column: Xselect CSH C¹⁸ OBD Column 30*150 mm 5 μm, n; Mobile Phase A: Water(0.1% FA), Mobile Phase B: ACN; Flow rate: 60 mL/min; Gradient: 15% B to 35% B in 8 min, 35% B; Wave Length: 254/220 nm; RT1(min): 8; Number Of Runs: 0. After lyophilization, it was afforded methyl2-{2-formyl-3-[(4-methoxyphenyl)methoxy]phenoxy}-N-[(2E)-3-(2-hydroxy-2-methylpropyl)-1H-imidazo[4,5-b]pyridin-2-ylidene]pyridine-4-carboxamide as a yellow solid (80 mg, 29.49% yield). MS (ESI) calcd. for C₃₉H₄₃N₅O₇, 693.32 m/z, found 694.32 [M+H]⁺.

### Step 3: Synthesis of methyl2-{2-formyl-3-[(4-methoxyphenyl)methoxy]phenoxy}-N-[(2E)-3-(2-hydroxy-2-methylpropyl)-1H-imidazo[4,5-b]pyridin-2-ylidene]pyridine-4-carboxamide

2-{2-formyl-3-[(4-methoxyphenyl)methoxy]phenoxy}-N-[(2E)-1-(2-hydroxy-2-methylpropyl)-6-{[3-(2-hydroxypropan-2-yl)azetidin-1-yl]methyl}-3H-1,3-benzodiazol-2-ylidene]pyridine-4-carboxamide(80 mg, 0.115 mmol), a stir bar, 2,2,2-trifluoroacetaldehyde acid (1 mL) were added to a 25-mL round-bottom flask and stirred until homogeneous. The resulting mixture was stirred at rt for 15 min. The residue was concentrated to dryness in vacuo to give a nearly yellow viscous oil. The crude product was then purified by Prep-HPLC with the following conditions: Column: X select CSH C¹⁸ OBD Column 30*150 mm 5 μm, n; Mobile Phase A: Water(0.1% FA), Mobile Phase B: ACN; Flow rate: 60 mL/min; Gradient: 34% B to 50% B in 10 min, 50% B; Wave Length: 254/220 nm; RT1(min): 10; Number Of Runs: 0. After lyophilization, it was afforded 2-(2-formyl-3-hydroxyphenoxy)-N-[(2E)-1-(2-hydroxy-2-methylpropyl)-6-{[3-(2-hydroxypropan-2-yl)azetidin-1-yl]methyl}-3H-1,3-benzodiazol-2-ylidene]pyridine-4-carboxamide (12.3 mg, 18.48% yield) as a white solid. MS (ESI) calcd. for C₃₁H₃₅N₅O₆, 573.26 m/z, found 574.30 [M+H]⁺.¹H NMR (300 MHz, DMSO-d⁶) δ (ppm): 10.22 (s, 1H), 8.26-8.28 (m, 2H), 7.80-7.82 (m, 1H), 7.46-7.68 (m, 3H), 7.15-7.18 (m, 1H), 6.87-6.89 (m, 1H), 6.67-6.70 (m, 1H), 4.10-4.33 (m, 2H), 3.33-4.09 (m, 2H), 3.27-3.32 (m, 2H), 3.12-3.15 (m, 2H), 2.43-2.51 (m, 1H), 1.14-1.21 (m, 6H), 0.87-1.00 (in, 6H).

The following compounds were synthesized according to the preceding method or analogous methods thereto:

| Cmpd. No. | Characterization Data |
|---|---|
| 74 | MS (ESI) calcd. for C28H29N5O6, 531.21 m/z, found 532.15 [M + H]+. 1H |
| | NMR (300 MHz, DMSO-d6) δ (ppm): 12.91 (s, 1H), 11.32 (s, 1H), 10.22 (s, |
| | 1H), 8.20-8.30 (m, 1H), 7.75-7.85 (m, 1H), 7.65-7.75 (m, 1H), 7.55-7.62 |
| | (m, 1H), 7.30-7.40 (m, 1H), 7.15-7.28 (m, 1H), 6.85-6.95 (m, 2H), 6.65- |
| | 6.77 (m, 1H), 4.18 (s, 2H), 3.77 (S, 4H), 3.13 (s, 4H), 1.21 (s, 6H). |
| 81 | MS (ESI) calcd. for C31H34N6O6, 586.25 m/z, found 587.30 [M + H]+. 1H |
| | NMR (400 MHz, DMSO-d6) δ (ppm): 12.95 (s, 1H), 10.28 (s, 1H), 9.81 (s, |
| | 1H), 8.31 (d, J = 1.2 Hz, 1H), 8.27 (s, 2H), 7.93 (d, J = 1.2 Hz, 1H), 7.65-7.56 |
| | (m, 2H), 7.51 (d, J = 8.2 Hz, 1H), 7.19 (d, J = 8.4 Hz, 1H), 6.94 (d, J = 8.4 Hz, |
| | 1H), 6.79 (d, J = 8.0 Hz, 1H), 4.19 (s, 2H), 3.54 (s, 2H), 2.45-2.38 (m, 4H), |
| | 2.28-2.25 (m, 2H), 2.16 (s, 4H), 1.23-1.21 (m, 1H), 1.21 (s, 6H). |
| 91 | MS (ESI) calcd. for C₃₄H₄₁N₇O₅, 627.32 m/z, found 628.35 [M + H]⁺. ¹H NMR |
| | (300 MHz, DMSO-d₆) δ 12.71 (s, 1H), 11.32 (s, 1H), 10.23 (s, 1H), 8.26 (d, J = |
| | 5.1 Hz, 1H), 7.81 (dd, J = 5.2, 1.3 Hz, 1H), 7.68 (d, J = 1.1 Hz, 1H), 7.59 (t, J = |
| | 8.3 Hz, 1H), 7.48 (d, J = 9.0 Hz, 1H), 7.13 (d, J = 2.3 Hz, 1H), 6.97 (dd, J = |
| | 9.0, 2.3 Hz, 1H), 6.88 (d, J = 8.4 Hz, 1H), 6.71 (dd, J = 8.1, 0.9 Hz, 1H), 4.94 |
| | (s, 1H), 4.15 (s, 2H), 3.64-3.50 (m, 2H), 2.67-2.41 (m, 11H), 2.36 (s, 3H), 1.90- |
| | 1.68 (m, 2H), 1.57-1.51 (m, 2H), 1.20 (s, 6H) |

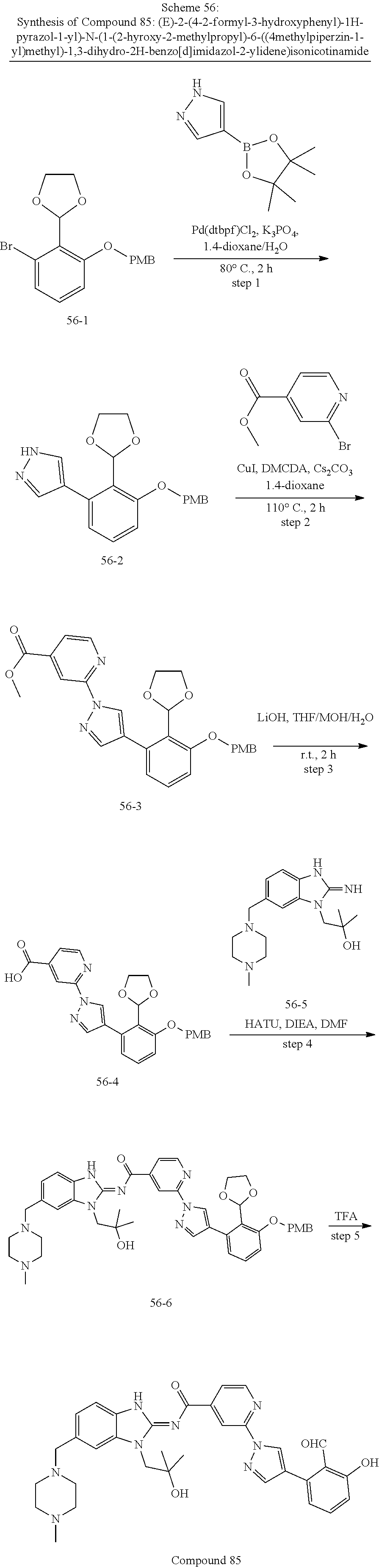

### Step 1: Synthesis of 4-(2-(1,3-dioxolan-2-yl)-3-((4-methoxybenzyl)oxy)phenyl)-1H-pyrazole

A mixture of 2-{2-bromo-6-[(4-methoxyphenyl)methoxy]phenyl}-1,3-dioxolane (4.9 g, 13.416 mmol, 1 equiv) 4-(4,4,5,5-tetramethyl-1,3,2-dioxaborolan-2-yl)-1H-pyrazole (3.90 g, 20.124 mmol, 1.5 equiv) K₃PO₄ (8.54 g, 40.248 mmol, 3 equiv) Pd(DtBPF)Cl₂ (1.31 g, 2.012 mmol, 0.15 equiv) in 1,4-dioxane (40 mL, 8.16 equiv) H₂O (10 mL, 2.04 equiv) was stirred for 2 h at 80° under N₂ atmosphere. The resulting mixture was filtered, the filter cake was washed with EA (3×20 ml). The filtrate was concentrated under reduced pressure. The residue/crude product was purified by reverse phase flash with the following conditions (Column: YMC-Actus Triart C18 ExR5, 30*150 mm, 5 μm; Mobile Phase A: Water(10 mmol/L NH₄HCO₃), Mobile Phase B: ACN; Flow rate: 60 mL/min; Gradient: 15% B to 30% B in 10.5 mn; Wave Length: 572; 220 nm; RT1(min): 10.5; Number Of Runs: 4) to afford 4-[2-(1,3-dioxolan-2-yl)-3-[(4-methoxyphenyl)methoxy]phenyl]-1H-pyrazole (2 g, 42.30%) as a white solid.353.10[M+H]⁺.

### Step 2: Synthesis of methyl 2-(4-(2-(1,3-dioxolan-2-yl)-3-((4-methoxybenzyl)oxy)phenyl)-1H-pyrazol-1-yl)isonicotinate

A solution of 4-[2-(1,3-dioxolan-2-yl)-3-[(4-methoxyphenyl)methoxy]phenyl]-1H-pyrazole (2 g, 5.676 mmol, 1 equiv) methyl 2-bromopyridine-4-carboxylate (1.23 g, 5.676 mmol, 1 equiv) CuI (0.22 g, 1.135 mmol, 0.2 equiv) Cs₂CO₃ (3.70 g, 11.352 mmol, 2 equiv) in 1,4-dioxane (20 mL, 10.00 equiv), followed by the addition of (1R,2R)—N1,N2-dimethylcyclohexane-1,2-diamine (0.32 g, 2.270 mmol, 0.4 equiv) The resulting mixture was stirred for 80° at 110° under N₂ atmosphere. The crude product (100 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 29% B in 8 min, 29% B; Wave Length: 254/220 nm; RT1(min): 8; Number Of Runs: 0) to afford methyl 2-{4-[2-(1,3-dioxolan-2-yl)-3-[(4-methoxyphenyl)methoxy]phenyl]pyrazol-1-yl}pyridine-4-carboxylate (1 g, 36.14%) as a yellow solid. LCMS:488.10[M+H]⁺.

### Step 3: Synthesis of 2-(4-(2-(1,3-dioxolan-2-yl)-3-((4-methoxybenzyl)oxy)phenyl)-1H-pyrazol-1-yl)isonicotinic acid

Into a 15 ml microwave tube were added methyl 2-{4-[2-(1,3-dioxolan-2-yl)-3-[(4-methoxyphenyl)methoxy]phenyl]pyrazol-1-yl}pyridine-4-carboxylate (1 g, 2.051 mmol, 1 equiv) lithiumol (0.15 g, 6.153 mmol, 3 equiv) and THF (3 mL, 3.00 equiv) H₂O (3 mL, 3.00 equiv) MeOH (3 mL, 3.00 equiv) at rt for 1h. The resulting mixture was extracted with EA (10 mL×3), dried over anhydrous Na₂SO₄. After filtration, the filtrate was concentrated under reduced pressure. The combined organic layers were washed with EQ (10 mL x 3). After filtration, the filtrate was concentrated under reduced pressure.to afford 2-{4-[2-(1,3-dioxolan-2-yl)-3-[(4-methoxyphenyl)methoxy]phenyl]pyrazol-1-yl}pyridine-4-carboxylic acid (600 mg, 61.78%) as a white solid. LCMS:473.16[M+H]⁺.

### Step 4: Synthesis of (E)-2-(4-(2-(1,3-dioxolan-2-yl)-3-((4-methoxybenzyl)oxy)phenyl)-1H-pyrazol-1-yl)-N-(1-(2-hydroxy-2-methylpropyl)-6-((4-methylpiperazin-1-yl)methyl)-1,3-dihydro-2H-benzo[d]imidazol-2-ylidene)isonicotinamide

A solution of 2-{4-[3-(benzyloxy)-2-(1,3-dioxolan-2-yl)phenyl]pyrazol-1-yl}pyridine-4-carboxylic acid (200 mg, 0.451 mmol, 1 equiv), HATU (205.78 mg, 0.541 mmol, 1.2 equiv), 1-{2-imino-6-[(4-methylpiperazin-1-yl)methyl]-3H-1,3-benzodiazol-1-yl}-2-methylpropan-2-ol (171.80 mg, 0.541 mmol, 1.2 equiv) in DMF (2 mL, 10.00 equiv) followed by the addition of DIEA (174.87 mg, 1.353 mmol, 3 equiv). The resulting mixture was stirred for 10 min at room temperature. The crude product (100 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 29% B in 8 min, 29% B; Wave Length: 254/220 nm; RT1(min): 8; Number Of Runs: 0) to afford 2-{4-[3-(benzyloxy)-2-formylphenyl]pyrazol-1-yl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (160 mg, 50.77%) LCMS:772.37[M+H]⁺.

### Step 5: Synthesis of (E)-2-(4-(2-formyl-3-hydroxyphenyl)-1H-pyrazol-1-yl)-N-(1-(2-hydroxy-2-methylpropyl)-6-((4-methylpiperazin-1-yl)methyl)-1,3-dihydro-2H-benzo[d]imidazol-2-ylidene)isonicotinamide

To a stirred solution of 2-{4-[2-(1,3-dioxolan-2-yl)-3-[(4-methoxyphenyl)methoxy]phenyl]pyrazol-1-yl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (160 mg, 0.207 mmol, 1 equiv) in TFA (2 mL, 12.50 equiv) at room temperature. The resulting mixture was stirred for 10 min at room temperature. The resulting mixture was concentrated under reduced pressure. The crude product (100 mg) was purified by Prep-HPLC with the following conditions (Column: XSelect CSH Fluoro Phenyl, 30*150 mm, 5 μm; Mobile Phase A: Water(0.1% FA), Mobile Phase B: ACN; Flow rate: 60 mL/min; Gradient: 5% B to 30% B in 9 min, 30% B; Wave Length: 254/220 nm; RT1(min): 9.03; Number Of Runs: 0) to afford 2-[4-(2-formyl-3-hydroxyphenyl)pyrazol-1-yl]-N-[(2E)-1-(2-hydroxy-2-methylpropyl)-6-[(4-methylpiperazin-1-yl)methyl]-3H-1,3-benzodiazol-2-ylidene]pyridine-4-carboxamide (37.9 mg, 29.90%) as a white solid. MS (ESI) calcd. for C₃₃H₃₆N₈O₄, 608.29 m/z, found 609.25 [M+H]⁺. 1H NMR (400 MHz, DMSO-d6) δ (ppm): 12.98 (s, 1H), 11.78 (s, 1H), 10.18 (s, 1H), 8.98 (s, 1H), 8.64-8.65 (m, 2H), 8.17-8.18 (m, 1H), 8.07-8.08 (m, 1H), 7.63-7.65 (m, 2H), 7.61-7.62 (m, 1H), 7.21-7.23 (m, 1H), 7.11-7.12 (m, 1H), 6.98-7.00 (m, 1H), 4.92 (s, 1H), 4.25 (s, 2H), 3.30-3.39 (m, 6H), 2.82-3.00 (m, 4H), 2.78-2.81 (m, 3H), 1.28 (s, 6H).

| Cmpd | |
|---|---|
| No. | Characterization Data |
| 102 | MS (ESI) calcd. for C44H50N8O6: 622.30 Found: 623.30 [M + H]⁺. ¹H NMR |
| | (300 MHz, DMSO-d⁶) δ (ppm): 12.93 (s, 1H), 11.86-11.87 (m, 1H), 10.18 (s, |
| | 1H), 8.92 (s, 1H), 8.47 (s, 1H), 8.15 (s, 1H), 7.89 (s, 1H), 7.63-7.65 (m, 2H), |
| | 7.52-7.58 (m, 1H), 7.13-7.19 (m, 2H), 6.97-7.00 (m, 1H), 4.93 (s, 1H), 4.2 |
| | (s, 2H), 3.59 (s, 2H), 2.73 (s, 3H), 2.24-2.37 (m, 8H), 2.17 (s, 3H), 1.10 (s, |
| | 3H). |

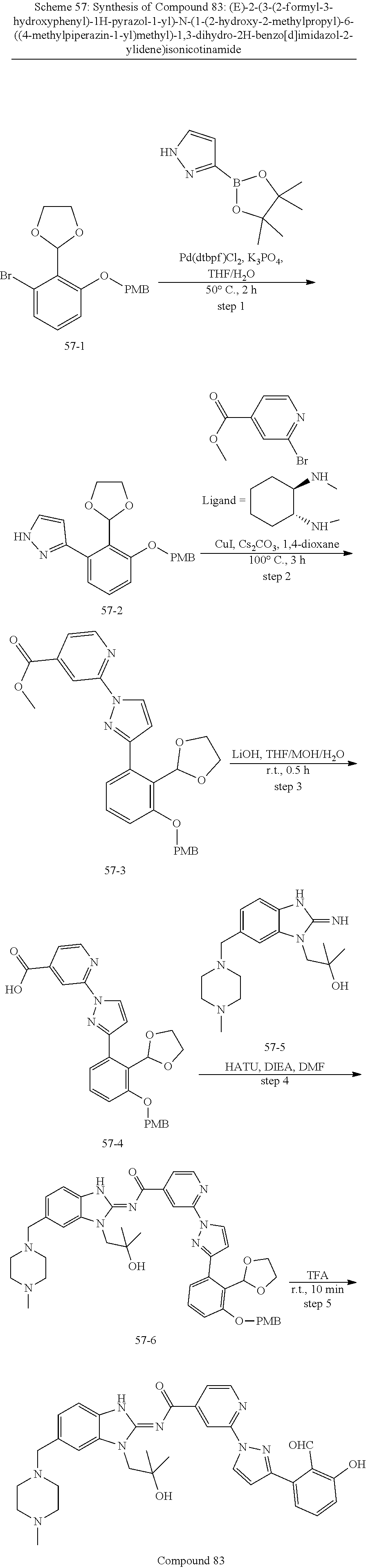

### Step 1: Synthesis of 3-(2-(1,3-dioxolan-2-yl)-3-((4-methoxybenzyl)oxy)phenyl)-1H-pyrazole

To a 50 ml round-bottomed flask equipped with a stirring bar were added 2-(2-bromo-6-((4-methoxybenzyl)oxy)phenyl)-1,3-dioxolane (1 g, 2.738 mmol) in 15 mL dioxane and 5 mL H₂O, followed by addition of 3-(4,4,5,5-tetramethyl-1,3,2-dioxaborolan-2-yl)-1H-pyrazole (1.2 g, 6.184 mmol), Pd(dtbpf)Cl₂ (0.55 g, 0.844 mmol) and K₃PO₄ (3 g, 14.133 mmol). The resulting solution was stirred at 50° C. for 2 h under the N2. The resulting mixture was extracted with EA (3 * 10 mL), dried over anhydrous Na₂SO₄. After filtration, the filtrate was concentrated under reduced pressure. The residue obtained was purified by reverse phase column (55% ACN) to afford the 3-(2-(1,3-dioxolan-2-yl)-3-((4-methoxybenzyl)oxy)phenyl)-1H-pyrazole a yellow solid (300 mg, 28.28% yield). MS (ESI) calcd. for C₂₀H₂₀N₂O₄, 352.14 m/z, found 353.20 [M+H]⁺.

### Step 2: Synthesis of methyl 2-(3-(2-(1,3-dioxolan-2-yl)-3-((4-methoxybenzyl)oxy)phenyl)-1H-pyrazol-1-yl)isonicotinate

To a 50 ml round-bottomed flask equipped with a stirring bar were added 3-(2-(1,3-dioxolan-2-yl)-3-((4-methoxybenzyl)oxy)phenyl)-1H-pyrazole (280 mg, 0.795 mmol) and 5 mL dioxane, followed by addition of methyl 2-bromoisonicotinate (200 mg, 0.926 mmo), CuI (20 mg, 0.105 mmo), (1R,2R)—N1,N2-dimethylcyclohexane-1,2-diamine (25 mg, 0.176 mmol) and caesio methaneperoxoate caesium (520 mg, 1.591 mmol). The resulting solution was stirred at 100° C. for 3 h under the N2. The resulting mixture was extracted with EA (3×10 mL), dried over anhydrous Na₂SO₄. After filtration, the filtrate was concentrated under reduced pressure.

The residue obtained was purified by reverse phase column (55% ACN) to afford the methyl 2-(3-(2-(1,3-dioxolan-2-yl)-3-((4-methoxybenzyl)oxy)phenyl)-1H-pyrazol-1-yl)isonicotinate a white solid (170 mg, 42.03% yield). MS (ESI) calcd. for C₂₇H₂₅N₃O₆, 487.17 m/z, found 488.25 [M+H]⁺.

### Step 3: Synthesis of 2-(3-(2-(1,3-dioxolan-2-yl)-3-((4-methoxybenzyl)oxy)phenyl)-1H-pyrazol-1-yl)isonicotinic acid

To a 25 ml round-bottomed flask equipped with a stirring bar were added methyl 2-(3-(2-(1,3-dioxolan-2-yl)-3-((4-methoxybenzyl)oxy)phenyl)-1H-pyrazol-1-yl)isonicotinate (170 mg, 0.349 mmol), 1 mL THF and 1 mL EtOH, followed by addition of LiOH (45 mg, 1.879 mmo) in 1 mL H₂O. The resulting solution was stirred at rt for 0.5 h. H₂O (5 mL) was added. The mixture was then adjusted to pH 6-7 and was extracted with EA (3 * 10 mL). The organic layers were combined, dried over anhydrous sodium sulfate, filtered and concentrated to dryness to give a white solid. It was afforded the 2-(3-(2-(1,3-dioxolan-2-yl)-3-((4-methoxybenzyl)oxy)phenyl)-1H-pyrazol-1-yl)isonicotinic acid as a white solid (150 mg, 88.77% yield). MS (ESI) calcd. for C₂₆H₂₃N₃O₆: 473.16, found: 474.25 [M+H]⁺.

### Step 4: Synthesis of (E)-2-(3-(2-(1,3-dioxolan-2-yl)-3-((4-methoxybenzyl)oxy)phenyl)-1H-pyrazol-1-yl)-N-(1-(2-hydroxy-2-methylpropyl)-6-((4-methylpiperazin-1-yl)methyl)-1,3-dihydro-2H-benzo[d]imidazol-2-ylidene)isonicotinamide

2-(3-(2-(1,3-dioxolan-2-yl)-3-((4-methoxybenzyl)oxy)phenyl)-1H-pyrazol-1-yl)isonicotinic acid (80 mg, 0.169 mmol) and 2 mL DMF, followed by addition of 1-(2-imino-6-((4-methylpiperazin-1-yl)methyl)-2,3-dihydro-1H-benzo[d]imidazol-1-yl)-2-methylpropan-2-ol (57.0 mg, 0.180 mmol), DIEA (65 mg, 0.503 mmol) and HATU (160 mg, 0.421 mmol). The resulting solution was stirred at rt for 0.5 h. The resulting mixture was extracted with EA (3 * 10 mL). The combined organic layers were washed with water (3 * 5 mL), dried over anhydrous Na₂SO₄. After filtration, the filtrate was concentrated under reduced pressure. It to afford (E)-2-(3-(2-(1,3-dioxolan-2-yl)-3-((4-methoxybenzyl)oxy)phenyl)-1H-pyrazol-1-yl)-N-(1-(2-hydroxy-2-methylpropyl)-6-((4-methylpiperazin-1-yl)methyl)-1,3-dihydro-2H-benzo[d]imidazol-2-ylidene)isonicotinamide as a yellow oil. (80 mg, 42.44% yield). MS (ESI) calcd. for C₄₃H₄₈N₈O₆, 772.37 m/z, found 729.55 [M+H]⁺.

### Step 5: Synthesis of (E)-2-(3-(2-formyl-3-hydroxyphenyl)-1H-pyrazol-1-yl)-N-(1-(2-hydroxy-2-methylpropyl)-6-((4-methylpiperazin-1-yl)methyl)-1,3-dihydro-2H-benzo[d]imidazol-2-ylidene)isonicotinamide

2-{3-[2-(1,3-dioxolan-2-yl)-3-[(4-methoxyphenyl)methoxy]phenyl]pyrazol-1-yl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (80 mg, 0.104 mmol, 1 equiv), a stir bar, trifluoroacetic acid (2 mL) were added to a 50-mL round-bottom flask and stirred until homogeneous. The resulting mixture was stirred at rt for 10 min. The residue was concentrated to dryness in vacuo to give a nearly yellow viscous oil. The crude product was then purified by Prep-HPLC with the following conditions: Column: Xselect CSH C18 OBD Column 30*150 mm 5 μm, n; Mobile Phase A: Water(0.1% FA), Mobile Phase B: ACN; Flow rate: 60 mL/min; Gradient: 20% B to 40% B in 8 min, 40% B; Wave Length: 254/220 nm; RT1(min): 8; Number Of Runs: 0. After lyophilization, it was afforded (E)-2-(3-(2-formyl-3-hydroxyphenyl)-1H-pyrazol-1-yl)-N-(1-(2-hydroxy-2-methylpropyl)-6-((4-methylpiperazin-1-yl)methyl)-1,3-dihydro-2H-benzo[d]imidazol-2-ylidene)isonicotinamide (17.8 mg, 28.07%) as a light yellow solid. MS (ESI) calcd. for C₃₃H₃₆N₈O₄, 608.29 m/z, found 609.30 [M+H]⁺. ¹H NMR (300 MHz, DMSO-d6) δ (ppm): 12.91 (s, 1H), 11.87 (s, 1H), 10.54 (s, 1H), 8.79-8.88 (m, 1H), 8.58-8.75 (m, 2H), 8.03-8.08 (m, 1H), 7.68-7.78 (m, 1H), 7.57 (s, 1H), 7.45-7.56 (m, 1H), 7.31-7.40 (m, 1H), 7.25-7.35 (m, 1H), 7.02-7.13 (m, 2H), 4.86 (s, 1H), 4.21 (s, 2H), 3.53 (s, 2H), 2.22-2.52 (m, 8H), 2.15 (s, 3H), 1.26 (s, 6H).

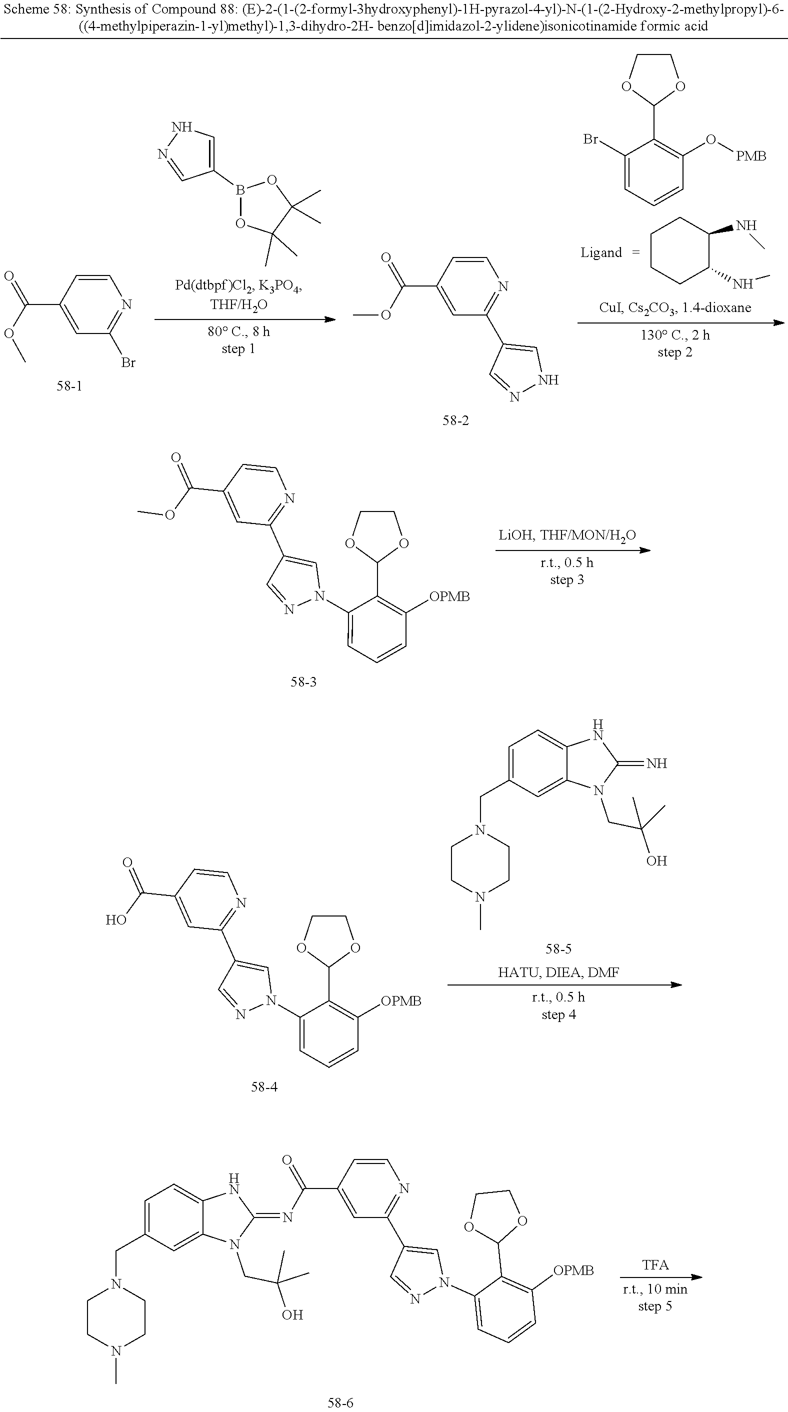

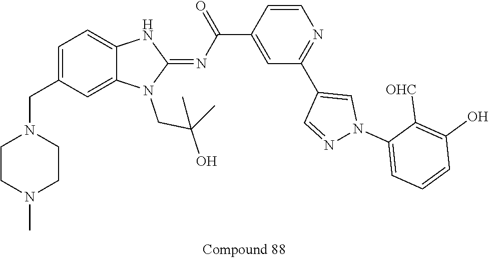 [SMILES: CN1CCN(Cc2ccc3[nH]/c(=N\C(=O)c4ccnc(-c5cnn(-c6cccc(O)c6C=O)c5)c4)n(CC(C)(C)O)c3c2)CC1]

### Step 1: Synthesis of methyl 2-(1H-pyrazol-4-yl)isonicotinate

To a 50 ml round-bottomed flask equipped with a stirring bar were added 2-(2-bromo-6-((4-methoxybenzyl)oxy)phenyl)-1,3-dioxolane (1 g, 2.738 mmol) in 15 mL dioxane and 5 mLH₂O, followed by addition of 3-(4,4,5,5-tetramethyl-1,3,2-dioxaborolan-2-yl)-1H-pyrazole (1.2 g, 6.184 mmol), Pd(dtbpf)Cl₂ (0.55 g, 0.844 mmol) and K₃PO₄ (3 g, 14.133 mmol). The resulting solution was stirred at 80° C. for 8 h under the N2. The resulting mixture was extracted with EA (3 * 10 mL), dried over anhydrous Na₂SO₄. After filtration, the filtrate was concentrated under reduced pressure. The residue obtained was purified by reverse phase column (55% ACN) to afford the 3-(2-(1,3-dioxolan-2-yl)-3-((4-methoxybenzyl)oxy)phenyl)-1H-pyrazole a yellow solid (300 mg, 28.28% yield). MS (ESI) calcd. for C₂₀H₂₀N₂O₄, 352.14 m/z, found 353.20 [M+H]⁺.

### Step 2: Synthesis of methyl 2-(1-(2-(1,3-dioxolan-2-yl)-3-((4-methoxybenzyl)oxy)phenyl)-1H-pyrazol-4-yl)isonicotinate

To a 50 ml round-bottomed flask equipped with a stirring bar were added methyl 2-(1H-pyrazol-4-yl)pyridine-4-carboxylate (350 mg, 1.722 mmol) and 10 mL dioxane, followed by addition of 2-{2-bromo-6-[(4-methoxyphenyl)methoxy]phenyl}-1,3-dioxolane (600 mg, 1.643 mmol), CuI (33 mg, 0.173 mmol), (1R,2R)—N1,N2-dimethylcyclohexane-1,2-diamine (49 mg, 0.344 mmol) and caesio methaneperoxoate caesium (1100 mg, 3.366 mmol). The resulting solution was stirred at 130° C. for 2 h under the N2. The resulting mixture was extracted with EA (3×10 mL), dried over anhydrous Na₂SO₄. After filtration, the filtrate was concentrated under reduced pressure. The residue obtained was purified by reverse phase column (55% ACN) to afford the methyl 2-(1-(2-(1,3-dioxolan-2-yl)-3-((4-methoxybenzyl)oxy)phenyl)-1H-pyrazol-4-yl)isonicotinate a white solid (150 mg, 10.99% yield). MS (ESI) calcd. for C₂₇H₂₅N₃O₆, 487.17 m/z, found 488.25 [M+H]+

### Step 3: Synthesis of 2-(1-(2-(1,3-dioxolan-2-yl)-3-((4-methoxybenzyl)oxy)phenyl)-1H-pyrazol-4-yl)isonicotinic acid

To a 25 ml round-bottomed flask equipped with a stirring bar were added methyl 2-{1-[2-(1,3-dioxolan-2-yl)-3-[(4-methoxyphenyl)methoxy]phenyl]pyrazol-4-yl}pyridine-4-carboxylate (150 mg, 0.308 mmol), 1 mL THF and 1 mL EtOH, followed by addition of LiOH (40 mg, 1.670 mmol) in 1 mL H₂O. The resulting solution was stirred at rt for 0.5 h.H₂O (5 mL) was added. The mixture was then adjusted to pH 6-7 and was extracted with EA (3 * 10 mL).

The organic layers were combined, dried over anhydrous sodium sulfate, filtered and concentrated to dryness to give a white solid. It was afforded the 2-(1-(2-(1,3-dioxolan-2-yl)-3-((4-methoxybenzyl)oxy)phenyl)-1H-pyrazol-4-yl)isonicotinic acid as a white solid (120 mg, 63.67% yield). MS (ESI) calcd. for C₂₆H₂₃N₃O₆: 473.16, found: 474.15 [M+H].

### Step 4: Synthesis of (E)-2-(1-(2-(1,3-dioxolan-2-yl)-3-((4-methoxybenzyl)oxy)phenyl)-1H-pyrazol-4-yl)-N-(1-(2-hydroxy-2-methylpropyl)-6-((4-methylpiperazin-1-yl)methyl)-1,3-dihydro-2H-benzo[d]imidazol-2-ylidene)isonicotinamide

2-{1-[2-(1,3-dioxolan-2-yl)-3-[(4-methoxyphenyl)methoxy]phenyl]pyrazol-4-yl}pyridine-4-carboxylic acid (80 mg, 0.129 mmol) and 3 mL DMF, followed by addition of 1-{2-imino-6-[(4-methylpiperazin-1-yl)methyl]-3H-1,3-benzodiazol-1-yl}-2-methylpropan-2-ol (57.0 mg, 0.180 mmol), DIEA (65 mg, 0.503 mmol) and HATU (120 mg, 0.316 mmol). The resulting solution was stirred at rt for 0.5 h. The resulting mixture was extracted with EA (3 * 10 mL). The combined organic layers were washed with water (3 * 10 mL), dried over anhydrous Na₂SO₄. After filtration, the filtrate was concentrated under reduced pressure. It to afford (E)-2-(1-(2-(1,3-dioxolan-2-yl)-3-((4-methoxybenzyl)oxy)phenyl)-1H-pyrazol-4-yl)-N-(1-(2-hydroxy-2-methylpropyl)-6-((4-methylpiperazin-1-yl)methyl)-1,3-dihydro-2H-benzo[d]imidazol-2-ylidene)isonicotinamide as a brrown oil. (80 mg, 53.9% yield). MS (ESI) calcd. for C₄₃H₄₈N₈O₆, 772.37 m/z, found 773.85 [M+H]⁺.

### Step 5: Synthesis of (E)-2-(1-(2-formyl-3-hydroxyphenyl)-1H-pyrazol-4-yl)-N-(1-(2-hydroxy-2-methylpropyl)-6-((4-methylpiperazin-1-yl)methyl)-1,3-dihydro-2H-benzo[d]imidazol-2-ylidene)isonicotinamide formic acid

2-{1-[2-(1,3-dioxolan-2-yl)-3-[(4-methoxyphenyl)methoxy]phenyl]pyrazol-4-yl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (100 mg, 0.129 mmol, 1 equiv), a stir bar, trifluoroacetic acid (2 mL) were added to a 50-mL round-bottom flask and stirred until homogeneous. The resulting mixture was stirred at rt for 10 min. The residue was concentrated to dryness in vacuo to give a nearly yellow viscous oil. The crude product was then purified by Prep-HPLC with the following conditions: Column: Xselect CSH C18 OBD Column 30*150 mm 5 μm, n; Mobile Phase A: Water(0.1% FA), Mobile Phase B: ACN; Flow rate: 60 mL/min; Gradient: 10% B in 8 min; Wave Length: 254/220 nm; RT1(min): 8; Number Of Runs: 0. After lyophilization, it was afforded (E)-2-(1-(2-formyl-3-hydroxyphenyl)-1H-pyrazol-4-yl)-N-(1-(2-hydroxy-2-methylpropyl)-6-((4-methylpiperazin-1-yl)methyl)-1,3-dihydro-2H-benzo[d]imidazol-2-ylidene)isonicotinamide formic acid (25.2 mg, 29.71%) as an off-white solid. MS (ESI) calcd. for C₃₃H₃₆N₈O₄, 608.29 m/z, found 609.30 [M+H]⁺. ¹H NMR (300 MHz, DMSO-d6) δ (ppm): 12.87 (s, 1H), 11.50 (s, 1H), 10.00 (s, 1H), 8.96 (s, 1H), 8.73 (m, 1H), 8.33-8.47 (m, 2H), 7.88-7.98 (m, 1H), 7.65-7.75 (m, 1H), 7.58 (s, 1H), 7.45-7.55 (m, 1H), 7.21-7.30 (m, 1H), 7.14-7.19 (m, 1H), 7.05-7.12 (m, 1H), 4.99 (s, 1H), 4.27 (s, 2H), 3.51 (s, 2H), 2.28-2.42 (m, 8H), 2.20 (s, 3H), 1.26 (s, 6H).

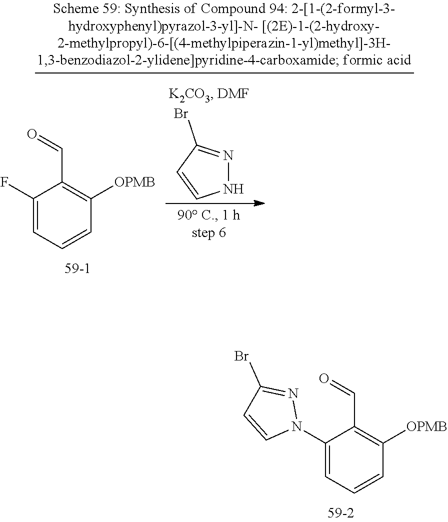

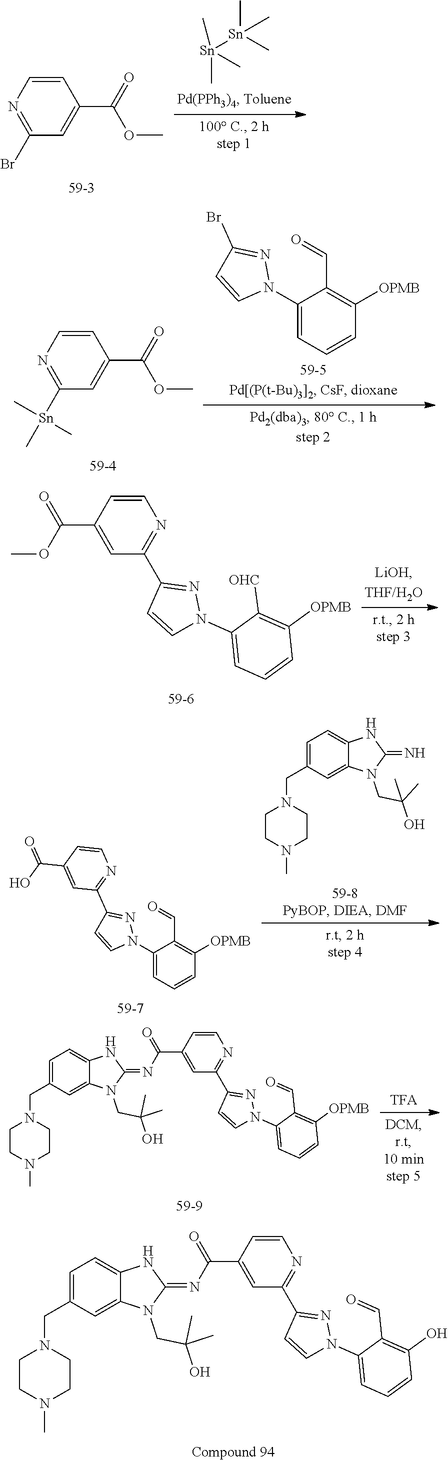

### Step 1: Synthesis of methyl 2-(trimethylstannyl)pyridine-4-carboxylate

A solution of methyl 2-bromopyridine-4-carboxylate (1 g, 4.62 mmol, 1 equiv.) and hexamethyldistannane (1.8 g, 5.55 mmol, 1.2 equiv.) in toluene (5 mL) was stirred for 2 h at 100° C. under N₂ atmosphere. Desired product was detected by LCMS. The resulting mixture was diluted with EA (100 mL) and washed with water (30 mL×3), dried over anhydrous Na₂SO₄. After filtration, the filtrate was concentrated under reduced pressure. The residue was purified by aluminum oxide column, eluted with EA/PE (0-20%) to afford methyl 2-(trimethylstannyl)pyridine-4-carboxylate (310 mg, 14.4%) as colorless oil. MS (ESI) calcd. for C₁₀H₁₅NO₂Sn, 301.01 m/z, found 301.90 [M+H].

### Step 2: Synthesis of methyl 2-(1-{2-formyl-3-[(4-methoxyphenyl)methoxy]phenyl}pyrazol-3-yl)pyridine-4-carboxylate

A solution of methyl 2-(trimethylstannyl)pyridine-4-carboxylate (100 mg, 0.33 mmol, 1 equiv), tri-tert-butyl[(tri-tert-butyl-lambda5-phosphanyl)palladio]-lambda5-phosphane (17.1 mg, 0.03 mmol, 0.1 equiv.), 2-(3-bromopyrazol-1-yl)-6-[(4-methoxyphenyl)methoxy]benzaldehyde (142.0 mg, 0.37 mmol, 1.1 equiv.), CsF (101.3 mg, 0.66 mmol, 2 equiv.) and Pd₂(dba)₃ (15.2 mg, 0.02 mmol, 0.05 equiv.) in dioxane (1 mL) was stirred for 1 h at 80° C. under N₂ atmosphere. Desired product was detected by LCMS. The resulting mixture was diluted with EA (100 mL) and washed with water (30 mL×3), dried over anhydrous Na₂SO₄. After filtration, the filtrate was concentrated under reduced pressure. The residue was purified by silica gel column chromatography, eluted with EA/PE (0-24%) to afford methyl 2-(1-{2-formyl-3-[(4-methoxyphenyl)methoxy]phenyl}pyrazol-3-yl)pyridine-4-carboxylate (28 mg, 12.2%) as a yellow solid. MS (ESI) calcd. for C₂₅H₂₁N₃O₅, 443.14 m/z, found 444.10 [M+H]⁺.

### Step 3: Synthesis of 2-(1-{2-formyl-3-[(4-methoxyphenyl)methoxy]phenyl}pyrazol-3-yl)pyridine-4-carboxylic acid

A solution of methyl 2-(1-{2-formyl-3-[(4-methoxyphenyl)methoxy]phenyl}pyrazol-3-yl)pyridine-4-carboxylate (28 mg, 0.06 mmol, 1 equiv.) and LiOH (0.04 mL, 0.126 mmol, 2 equiv., 3 M in H₂O) in THF (1 mL) and H₂O (0.2 mL) was stirred for 2 h at 25° C. Desired product was detected by LCMS. The mixture was neutralized to pH 7 with HCl (2 mol/L) and then concentrated under reduced pressure to afford 2-(1-{2-formyl-3-[(4-methoxyphenyl)methoxy]phenyl}pyrazol-3-yl)pyridine-4-carboxylic acid (20 mg, 66%) as a white solid. MS (ESI) calcd. for C₂₄H₁₉N₃O₅, 429.13 m/z, found 430.15 [M+H]⁺.

### Step 4: Synthesis of 2-(1-{2-formyl-3-[(4-methoxyphenyl)methoxy]phenyl}pyrazol-3-yl)-N-[(2E)-1-(2-hydroxy-2-methylpropyl)-6-[(4-methylpiperazin-1-yl)methyl]-3H-1,3-benzodiazol-2-ylidene]pyridine-4-carboxamide

A solution of 2-(1-{2-formyl-3-[(4-methoxyphenyl)methoxy]phenyl}pyrazol-3-yl)pyridine-4-carboxylic acid (40 mg, 0.09 mmol, 1.1 equiv.), 1-{2-imino-6-[(4-methylpiperazin-1-yl)methyl]-3H-1,3-benzodiazol-1-yl}-2-methylpropan-2-ol (26.8 mg, 0.09 mmol, 1 equiv.), PyBOP (66.1 mg, 0.13 mmol, 1.5 equiv.) and DIEA (21.9 mg, 0.17 mmol, 2 equiv.) in DMF (2 mL) was stirred for 16 h at 25° C. Desired product was detected by LCMS. The residue was purified by reverse flash chromatography with MeCN/H₂O (0.5% TFA) from 0% to 60% to afford 2-(1-{2-formyl-3-[(4-methoxyphenyl)methoxy]phenyl}pyrazol-3-yl)-N-[(2E)-1-(2-hydroxy-2-methylpropyl)-6-[(4-methylpiperazin-1-yl)methyl]-3H-1,3-benzodiazol-2-ylidene]pyridine-4-carboxamide (24 mg, 18.2%) as a yellow solid. MS (ESI) calcd. for C₄₁H₄₄N₈O₅, 728.34 m/z, found 729.35[M+H]⁺.

### Step 5: Synthesis of 2-[1-(2-formyl-3-hydroxyphenyl)pyrazol-3-yl]-N-[(2E)-1-(2-hydroxy-2-methylpropyl)-6-[(4-methylpiperazin-1-yl)methyl]-3H-1,3-benzodiazol-2-ylidene]pyridine-4-carboxamide; formic acid

A solution of 2-(1-{2-formyl-3-[(4-methoxyphenyl)methoxy]phenyl}pyrazol-3-yl)-N-[(2E)-1-(2-hydroxy-2-methylpropyl)-6-[(4-methylpiperazin-1-yl)methyl]-3H-1,3-benzodiazol-2-ylidene]pyridine-4-carboxamide (24 mg, 0.03 mmol, 1 equiv.) in TFA (0.2 mL) and DCM (0.6 mL) was stirred for 10 min at rt. The desired product was detected by LCMS. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 12% B to 36% B in 9 min, 36% B; Wave Length: 254/220 nm; RT1(min): 7.6; Number Of Runs: 0) to afford 2-[1-(2-formyl-3-hydroxyphenyl)pyrazol-3-yl]-N-[(2E)-1-(2-hydroxy-2-methylpropyl)-6-[(4-methylpiperazin-1-yl)methyl]-3H-1,3-benzodiazol-2-ylidene]pyridine-4-carboxamide (2 mg, 11.9%) as a grey solid. MS (ESI) calcd. for C₃₃H₃₆N₈O₄, 608.29 m/z, found 609.10 [M+H]⁺. ¹H NMR (400 MHz, DMSO-d₆) δ (ppm): 12.88 (s, 1H), 10.11 (s, 1H), 8.78 (d, J=5.1 Hz, 1H), 8.72 (s, 1H), 8.46 (d, J=2.5 Hz, 1H), 8.03 (d, J=5.0 Hz, 1H), 7.71 (t, J=8.2 Hz, 1H), 7.58 (s, 1H), 7.50 (d, J=8.1 Hz, 1H), 7.25 (d, J=7.9 Hz, 1H), 7.22-7.14 (m, 2H), 7.11 (d, J=8.4 Hz, 1H), 4.90 (s, 1H), 4.22 (s, 3H), 3.57 (s, 3H), 2.41 (s, 4H), 2.28 (s, 4H), 1.23 (s, 8H).

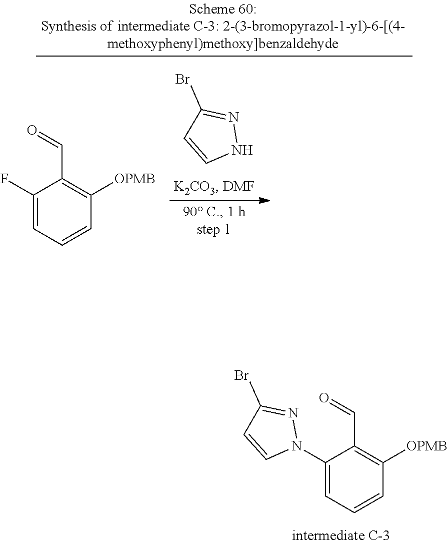

### Step 1: Synthesis of 2-(3-bromopyrazol-1-yl)-6-[(4-methoxyphenyl)methoxy]benzaldehyde

A solution of 3-bromo-1H-pyrazole (1 g, 6.804 mmol, 1 equiv.), 2-fluoro-6-[(4-methoxyphenyl)methoxy]benzaldehyde (1.77 g, 6.804 mmol, 1 equiv.) and Cs₂CO₃ (3.33 g, 10.206 mmol, 1.5 equiv.) in ACN (10.00 mL) was stirred for 16 h at 60° C. Desired product was detected by LCMS. The resulting mixture was extracted with EA (150 mL×3). The combined organic layers were washed with water (50 mL×3), dried over anhydrous Na₂SO₄. After filtration, the filtrate was concentrated under reduced pressure. The residue was purified by silica gel column chromatography, eluted with EA/PE (0-25%) to afford crude product. The residue was purified by reverse flash chromatography with MeCN/H₂O (0.05% NH₄HCO₃) from 65% to afford 2-(3-bromopyrazol-1-yl)-6-[(4-methoxyphenyl)methoxy]benzaldehyde (260 mg, 9.40%) as a yellow solid. MS (ESI) calcd. for C₁₈H₁₅BrN₂O₃, 386.02 m/z, found 388.95 [M+H+2]⁺.

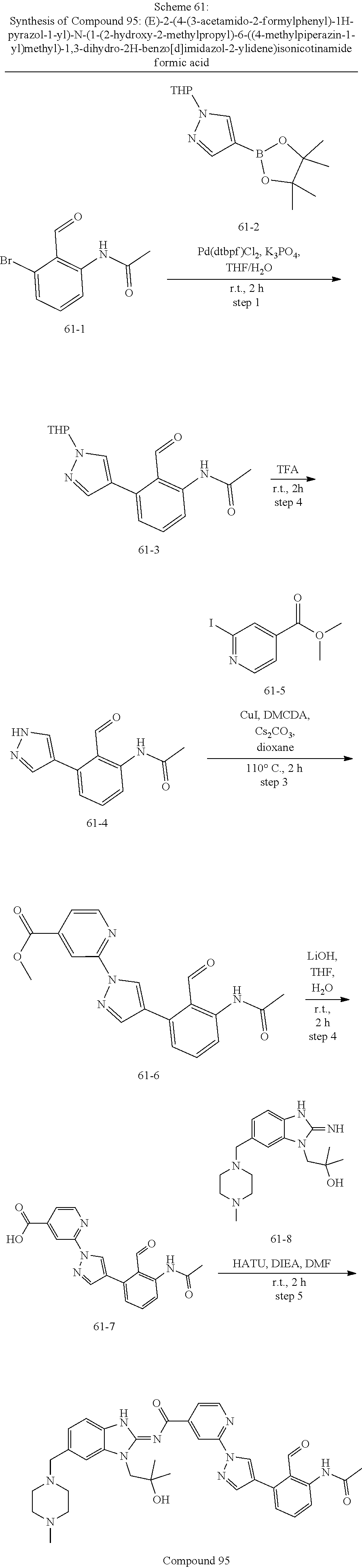

### Step 1: Synthesis of N-(2-formyl-3-(1-(tetrahydro-2H-pyran-2-yl)-1H-pyrazol-4-yl)phenyl)acetamide

A solution of N-[3-bromo-2-(1,3-dioxolan-2-yl)phenyl]acetamide (1 g, 3.495 mmol, 1 equiv) 1-(oxan-2-yl)-4-(4,4,5,5-tetramethyl-1,2-oxaborolan-2-yl)pyrazole (1.16 g, 4.194 mmol, 1.2 equiv) K₃PO₄ (2.23 g, 10.485 mmol, 3 equiv) in THF (12 mL, 12.00 equiv) H₂O (3 mL, 3.00 equiv), followed by the addition of Pd(DtBPF)Cl₂ (0.34 g, 0.524 mmol, 0.15 equiv). The resulting mixture was stirred for 10 min at room temperature under N₂ atmosphere. The crude product (100 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 29% B in 8 min, 29% B; Wave Length: 254/220 nm; RT1(min): 8; Number Of Runs: 0) to afford N-[2-formyl-3-(1H-pyrazol-4-yl)phenyl]acetamide (600 mg, 74.89%) as a white solid. MS (ESI) calcd. for C₁₇H₁₉N₃O₃, 313.14 m/z, found 315.09[M+H]⁺.

### Step 2: Synthesis of N-(2-formyl-3-(1H-pyrazol-4-yl)phenyl)acetamide

To a stirred solution of N-{2-formyl-3-[1-(oxan-2-yl)pyrazol-4-yl]phenyl}acetamide (600 mg, 1.915 mmol, 1 equiv) in TFA (8 mL, 0.082 mmol) at room temperature. The resulting mixture was stirred for 10 min at room temperature. The resulting mixture was concentrated under reduced pressure. The crude product (100 mg) was purified by Prep-HPLC with the following conditions (Column: XBridge Prep OBD C18 Column, 30*150 mm, 5 μm; Mobile Phase A: Water(0.1% FA), Mobile Phase B: ACN; Flow rate: 60 mL/min; Gradient: 26% B to 42% B in 9 min, 42% B; Wave Length: 254/220 nm; RT1(min): 8.9; Number Of Runs: 0) to afford N-[2-formyl-3-(1H-pyrazol-4-yl)phenyl]acetamide as a White solid. MS (ESI) calcd. for C₁₂H₁₁N₃O_{2-, 229.09} m/z, found 230.10[M+H]⁺.

### Step 3: Synthesis of methyl 2-(4-(3-acetamido-2-formylphenyl)-1H-pyrazol-1-yl)isonicotinate

A solution of N-[2-(1,3-dioxolan-2-yl)-3-(1H-pyrazol-4-yl)phenyl]acetamide (300 mg, 1.098 mmol, 1 equiv) methyl 2-bromopyridine-4-carboxylate (284.58 mg, 1.318 mmol, 1.2 equiv) CuI (31.36 mg, 0.165 mmol, 0.15 equiv) Cs₂CO₃ (715.32 mg, 2.196 mmol, 2 equiv) in 1,4-dioxane (5 mL, 16.67 equiv),followed by the addition of ((1R,2R)-1-N, 1-N-dimethylcyclohexane-1,2-diamine (46.84 mg, 0.329 mmol, 0.3 equiv) The resulting mixture was stirred for 10 min at room temperature under N₂ atmosphere. The crude product (100 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 29% B in 8 min, 29% B; Wave Length: 254/220 nm; RT1(min): 8; Number Of Runs: 0) to afford methyl 2-[4-(3-acetamido-2-formylphenyl)pyrazol-1-yl]pyridine-4-carboxylate (190 mg, 47.50%) as a white solid. MS (ESI) calcd. for C₁₉H₁₆N₄O₄, 364.12 m/z, found 365.15 [M+H]⁺.

### Step 4: Synthesis of 2-(4-(3-acetamido-2-formylphenyl)-1H-pyrazol-1-yl)isonicotinic acid

Into a 15 ml microwave tube were added methyl 2-[4-(3-acetamido-2-formylphenyl)pyrazol-1-yl]pyridine-4-carboxylate (190 mg, 0.521 mmol, 1 equiv), lithiumol (37.47 mg, 1.563 mmol, 3 equiv), and THF (2 mL, 10.53 equiv) H₂O (2 mL, 10.53 equiv) at rt for 1h. The resulting mixture was extracted with EA (10 ml×3). dried over anhydrous NA₂SO₄. After filtration, the filtrate was concentrated under reduced pressure. The combined organic layers were washed with EA(10 ml×3). After filtration, the filtrate was concentrated under reduced pressure to afford 2-[4-(3-acetamido-2-formylphenyl)pyrazol-1-yl]pyridine-4-carboxylic acid (100 mg, 54.74%) as a white solid. MS (ESI) calcd. for C18H₁₄N₄O₄, 350.10 m/z, found 351.20 [M+H].

### Step 5: Synthesis of (E)-2-(4-(3-acetamido-2-formylphenyl)-1H-pyrazol-1-yl)-N-(1-(2-hydroxy-2-methylpropyl)-6-((4-methylpiperazin-1-yl)methyl)-1,3-dihydro-2H-benzo[d]imidazol-2-ylidene)isonicotinamide formic acid

A solution of 2-[4-(3-acetamido-2-formylphenyl)pyrazol-1-yl]pyridine-4-carboxylic acid (100 mg, 0.285 mmol, 1 equiv), 1-{2-imino-6-[(4-methylpiperazin-1-yl)methyl]-3H-1,3-benzodiazol-1-yl}-2-methylpropan-2-ol (108.73 mg, 0.342 mmol, 1.2 equiv), HATU (130.24 mg, 0.342 mmol, 1.2 equiv) in DMF (2 mL, 20.00 equiv), followed by the addition of DIEA (110.68 mg, 0.855 mmol, 3 equiv) The resulting mixture was stirred for 10 min at room temperature. The crude product (100 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 29% B in 8 min, 29% B; Wave Length: 254/220 nm; RT1(min): 8; Number Of Runs: 0) to afford 2-[4-(3-acetamido-2-formylphenyl)pyrazol-1-yl]-N-[(2E)-1-(2-hydroxy-2-methylpropyl)-6-[(4-methylpiperazin-1-yl)methyl]-3H-1,3-benzodiazol-2-ylidene]pyridine-4-carboxamide; formic acid (24.8 mg, 12.20%) as a white solid. MS (ESI) calcd. for C₃₅H₃₉N₉O₄, 649.31 m/z, found 650.35 [M+H]. ¹H NMR (300 MHz, DMSO-d⁶) δ (ppm): 12.98 (s, 1H), 11.27 (s, 1H), 10.12 (m, 1H), 9.20˜9.30 (m, 1H), 8.93 (s, 1H), 8.65-8.66 (m, 2H), 8.38-8.41 (m, 1H), 8.07-8.09 (m, 1H), 7.69-7.74 (m, 1H), 7.56-7.62 (m, 2H), 7.34-7.37 (m, 1H), 7.21-7.24 (m, 1H), 4.92 (s, 1H), 4.26 (s, 2H), 3.67 (s, 2H), 3.01-3.35 (m, 6H), 2.80 (s, 3H), 2.27-2.30 (s, 2H), 2.19 (s, 3H). 1.28 (s, 6H).

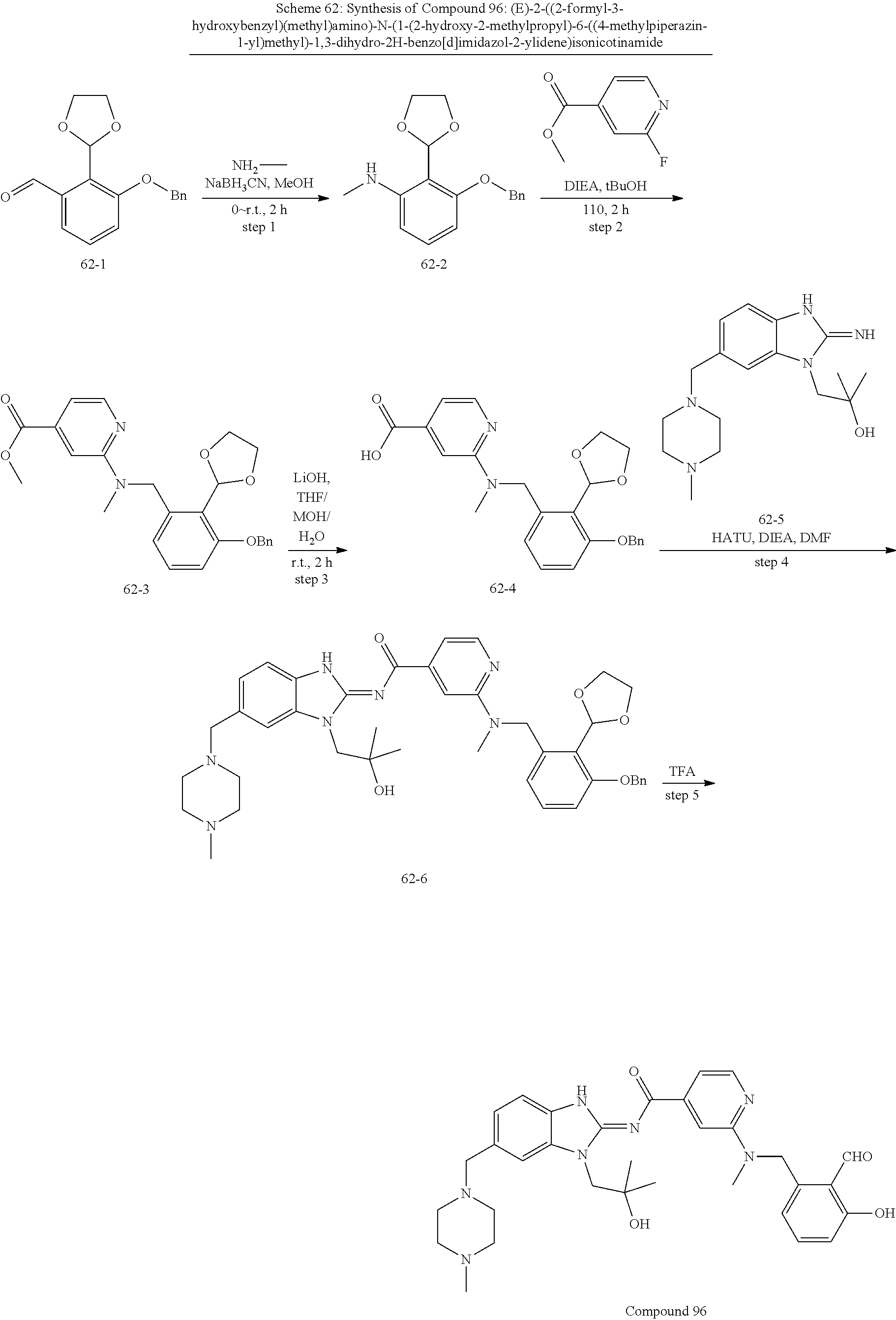

### Step 1: Synthesis of 3-(benzyloxy)-2-(1,3-dioxolan-2-yl)-N-methylaniline

Into a 50 ml round-bottomed flask were added 3-(benzyloxy)-2-(1,3-dioxolan-2-yl)benzaldehyde (1.5 g, 5.276 mmol, 1 equiv) and methylamine (1.31 g, 42.208 mmol, 8 equiv) in MeOH (20 mL, 13.33 equiv) at 0° C. The aqueous layer was extracted with EA (3×50 ml).The resulting mixture was concentrated under vacuum. The residue/crude product was purified by reverse phase flash chromatography with the following conditions (Column: XSelect CSH Fluoro Phenyl, 30*150 mm, 5 μm; Mobile Phase A: Water(0.1I % NH₄HCO₃), Mobile Phase B: ACN; Flow rate: 60 mL/min; Gradient: 5% B to 30% B in 9 min, 30% B; Wave Length: 254/220 nm; RT1(min): 9.03; Number Of Runs: 0) to afford {[3-(benzyloxy)-2-(1,3-dioxolan-2-yl)phenyl]methyl}(methyl)amine (720 mg, 44.67%) as a yellow solid.

### Step 2: Synthesis of methyl 2-((3-(benzyloxy)-2-(1,3-dioxolan-2-yl)benzyl)(methyl)amino)isonicotinate

Into a 10 mL microwave tube was added {[3-(benzyloxy)-2-(1,3-dioxolan-2-yl)phenyl]methyl}(methyl)amine (300 mg, 1.002 mmol, 1 equiv) methyl 2-fluoropyridine-4-carboxylate (310.91 mg, 2.004 mmol, 2 equiv) DIEA (518.08 mg, 4.008 mmol, 4 equiv) and tert-butanol (5 mL, 16.67 equiv) at 110° under N₂, The resulting mixture was concentrated under reduced pressure. The crude product (100 mg) was purified by Prep-HPLC with the following conditions (Column: XSelect CSH Fluoro Phenyl, 30*150 mm, 5 μm; Mobile Phase A: Water(0.1% FA), Mobile Phase B: ACN; Flow rate: 60 mL/min; Gradient: 5% B to 30% B in 9 min, 30% B; Wave Length: 254/220 nm; RT1(min): 9.03; Number Of Runs: 0) to afford methyl 2-({[3-(benzyloxy)-2-(1,3-dioxolan-2-yl)phenyl]methyl}(methyl)amino)pyridine-4-carboxylate (160 mg, 36.75%) as white solid. LCMS:435.10[M+H]⁺.

### Step 3: Synthesis of 2-((3-(benzyloxy)-2-(1,3-dioxolan-2-yl)benzyl)(methyl)amino)isonicotinic acid

Into a 15 ml microwave tube were added methyl 2-({[3-(benzyloxy)-2-(1,3-dioxolan-2-yl)phenyl]methyl}(methyl)amino)pyridine-4-carboxylate (160 mg, 0.368 mmol, 1 equiv), lithiumol (26.46 mg, 1.104 mmol, 3 equiv) and MeOH (1 mL, 6.25 equiv), H₂O (1 mL, 6.25 equiv) THF (1 mL, 6.25 equiv) at rt for 1h. The resulting mixture was extracted with EA (10 ml×3) and dried over anhydrous Na₂SO₄. After filtration, the filtrate was concentrated under reduced pressure. The combined organic layers were washed with EA (10 ml×3). After filtration, the filtrate was concentrated under reduced pressure to afford 2-({[3-(benzyloxy)-2-(1,3-dioxolan-2-yl)phenyl]methyl}(methyl)amino)pyridine-4-carboxylic acid (30 mg, 19.38%) as a white solid. LCMS:421.50[M+H]⁺.

### Step 4: Synthesis of (E)-2-((3-(benzyloxy)-2-(1,3-dioxolan-2-yl)benzyl)(methyl)amino)-N-(1-(2-hydroxy-2-methylpropyl)-6-((4-methylpiperazin-1-yl)methyl)-1,3-dihydro-2H-benzo[d]imidazol-2-ylidene)isonicotinamide

A solution of 2-({[3-(benzyloxy)-2-(1,3-dioxolan-2-yl)phenyl]methyl}(methyl)amino)pyridine-4-carboxylic acid (30 mg, 0.071 mmol, 1 equiv) 1-{2-imino-6-[(4-methylpiperazin-1-yl)methyl]-3H-1,3-benzodiazol-1-yl}-2-methylpropan-2-ol (24.91 mg, 0.078 mmol, 1.1 equiv) HATU (32.56 mg, 0.085 mmol, 1.2 equiv) in DMF (1 mL, 33.33 equiv), followed by the addition of DIEA (27.67 mg, 0.213 mmol, 3 equiv). The resulting mixture was stirred for 10 min at room temperature. The crude product (100 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 29% B in 8 min, 29% B; Wave Length: 254/220 nm; RT1(min): 8; Number Of Runs: 0) to afford 2-({[2-(1,3-dioxolan-2-yl)-3-[(4-methoxyphenyl)methoxy]phenyl]methyl}(methyl)amino)-N-[(2E)-1-(2-hydroxy-2-methylpropyl)-6-[(4-methylpiperazin-1-yl)methyl]-3H-1,3-benzodiazol-2-ylidene]pyridine-4-carboxamide (30 mg, 56.07%) as a white solid. LCMS:720.40[M+H]⁺.

### Step 5: Synthesis of (E)-2-((2-formyl-3-hydroxybenzyl)(methyl)amino)-N-(1-(2-hydroxy-2-methylpropyl)-6-((4-methylpiperazin-1-yl)methyl)-1,3-dihydro-2H-benzo[d]imidazol-2-ylidene)isonicotinamide

To a stirred solution of 2-({[3-(benzyloxy)-2-(1,3-dioxolan-2-yl)phenyl]methyl}(methyl)amino)-N-[(2E)-1-(2-hydroxy-2-methylpropyl)-6-[(4-methylpiperazin-1-yl)methyl]-3H-1,3-benzodiazol-2-ylidene]pyridine-4-carboxamide (30 mg, 0.042 mmol, 1 equiv) in TFA (0.8 mL, 0.167 mmol) CH₃SO₃H (0.2 mL, 0.042 mmol) at room temperature.The resulting mixture was stirred for 10 min at room temperature.The resulting mixture was concentrated under reduced pressure. The crude product (30 mg) was purified by Prep-HPLC with the following conditions (Column: XSelect CSH Fluoro Phenyl, 30*150 mm, 5 μm; Mobile Phase A: Water(0.1% FA), Mobile Phase B: ACN; Flow rate: 60 mL/min; Gradient: 5% B to 30% B in 9 min, 30% B; Wave Length: 254/220 nm; RT1(min): 9.03; Number Of Runs: 0) to afford 2-{[(2-formyl-3-hydroxyphenyl)methyl](methyl)amino}-N-[(2E)-1-(2-hydroxy-2-methylpropyl)-6-[(4-methylpiperazin-1-yl)methyl]-3H-1,3-benzodiazol-2-ylidene]pyridine-4-carboxamide (1 mg, 4.06%) as a light yellow solid. MS (ESI) calcd. for C₃₂H₃₉N₇O₄, 585.31 m/z, found 586.30 [M+H]⁺. 1H NMR (300 MHz, DMSO-d₆) δ (ppm): 12.88 (s, 1H), 11.10 (s, 1H), 10.56 (s, 1H), 8.16-8.18 (s, 1H), 7.51-7.58 (m, 1H), 7.44-7.48 (m, 1H), 7.39-7.41 (m, 1H), 7.32-7.34 (m, 2H), 7.19-7.22 (m, 1H), 6.91-6.94 (m, 1H), 6.59-6.62 (m, 1H), 5.16 (s, 2H), 4.09 (s, 2H), 3.68-3.78 (m, 2H), 3.22-3.33 (m, 4H), 3.02-3.03 (m, 4H), 2.76-2.77 (m, 3H) 2.31 (s, 3H), 1.05-1.09 (m, 6H).

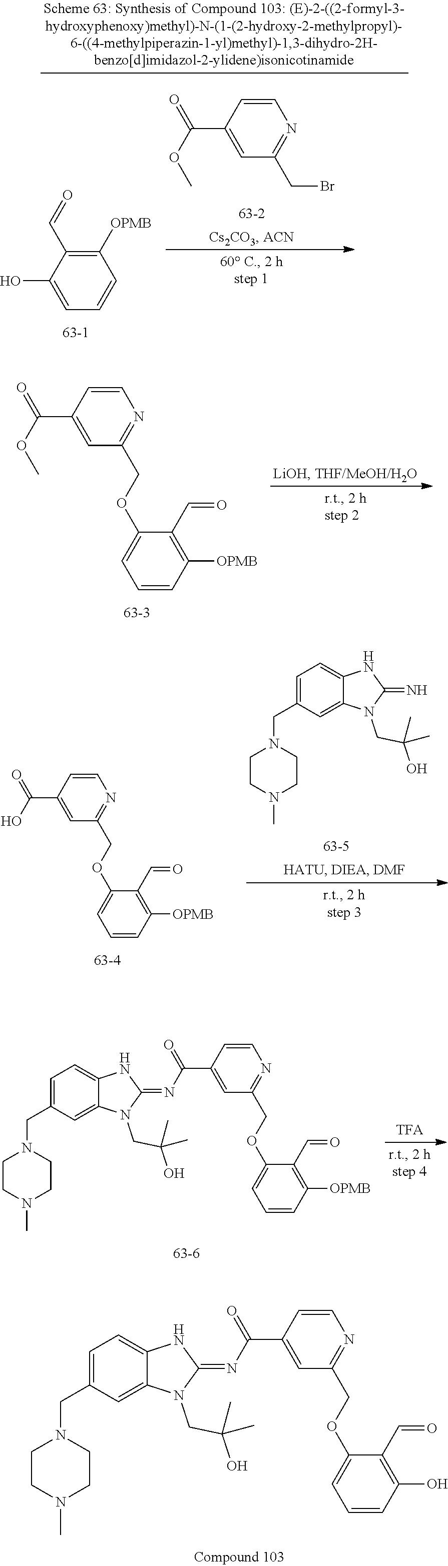

### Step 1: Synthesis of methyl 2-((2-formyl-3-((4-methoxybenzyl)oxy)phenoxy)methyl) isonicotinate

To a 50 ml round-bottomed flask equipped with a stirring bar were added methyl 2-hydroxy-6-[(4-methoxyphenyl)methoxy]benzaldehyde (300 mg, 1.162 mmol, 1 equiv) and ACN (4 mL, 76.097 mmol, 65.51 equiv), followed by addition of methyl 2-(bromomethyl)pyridine-4-carboxylate (320.68 mg, 1.394 mmol, 1.2 equiv) and Cs₂CO₃ (756.92 mg, 2.324 mmol, 2 equiv) at 0° C. The resulting solution was stirred at 60° C. for 2 h. The reaction progress was monitored by LCMS, and it showed the reaction was completed. The mixture was allowed to cool down to 25° C. The reaction was quenched with 100 mL H₂O at 25° C. The resulting mixture was extracted with EA (3×100 mL). Dried over anhydrous Na₂SO₄. After filtration, the filtrate was concentrated under reduced pressure. The residue was purified by silica gel column chromatography, eluted with PE/EA(5%) to afford methyl 2-{2-formyl-3-[(4-methoxyphenyl)methoxy]phenoxymethyl}pyridine-4-carboxylate (150 mg, 29.26%) as white a solid. LC/MS: MS (ESI) calcd. for C₂₃H₂₁NO₆: 407.14. Found: 408.14 [M+H]⁺.

### Step 2: Synthesis of 2-((2-formyl-3-((4-methoxybenzyl)oxy)phenoxy)methyl)isonicotinic acid

To a 25 ml round-bottomed flask equipped with a stirring bar were added methyl methyl 2-{2-formyl-3-[(4-methoxyphenyl)methoxy]phenoxymethyl}pyridine-4-carboxylate (150 mg, 0.368 mmol, 1 equiv), THF (2 mL, 24.686 mmol, 67.05equiv), followed by addition of LiOH (17.64 mg, 0.736 mmol, 2 equiv) in H₂O (0.7 mL, 38.857 mmol, 105.54equiv). The resulting solution was stirred at rt for 0.5 h. The mixture was acidified neutralized to pH 7 with HCl(2M). The solvents was evaporated to afford 2-{2-formyl-3-[(4-methoxyphenyl)methoxy] phenoxymethyl}pyridine-4-carboxylic acid (110 mg, 75.95%)LC/MS: MS (ESI) calcd. for C₂₂H₁₉NO₆: 393.12. Found: 394.10 [M+H]⁺..

### Step 3: Synthesis of (E)-2-((2-formyl-3-((4-methoxybenzyl)oxy)phenoxy)methyl)-N-(1-(2-hydroxy-2-methylpropyl)-6-((4-methylpiperazin-1-yl)methyl)-1,3-dihydro-2H-benzo[d]imidazol-2-ylidene)isonicotinamide

A solution of 2-{2-formyl-3-[(4-methoxyphenyl)methoxy]phenoxymethyl}pyridine-4-carboxylic acid (110 mg, 0.280 mmol, 1 equiv), 1-{2-imino-6-[(4-methylpiperazin-1-yl)methyl]-3H-1,3-benzodiazol-1-yl}-2-methylpropan-2-ol (97.64 mg, 0.308 mmol, 1.1 equiv), HATU (159.48 mg, 0.420 mmol, 1.5 equiv), followed by the addition of DIEA (72.28 mg, 0.560 mmol, 2 equiv). The residue was purified by reversed-phase flash chromatography with the following conditions: column, C18 silica gel; mobile phase, H₂O in ACN, 10% to 50% gradient in 15 min; detector, UV 254 nm. to afford 2-{2-formyl-3-[(4-methoxyphenyl)methoxy]phenoxymethyl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (50 mg, 21.94%) as a white solid. MS (ESI) calcd. for C₃₉H₄₄N₆O₆, 692.33 m/z, found: 693.33[M+H]⁺.

### Step 4: Synthesis of (E)-2-((2-formyl-3-hydroxyphenoxy)methyl)-N-(1-(2-hydroxy-2-methylpropyl)-6-((4-methylpiperazin-1-yl)methyl)-1,3-dihydro-2H-benzo[d]imidazol-2-ylidene)isonicotinamide

To a stirred solution of 2-{2-formyl-3-[(4-methoxyphenyl)methoxy]phenoxymethyl}-N-[(2E)-1-(2-hydroxy-2-methylpropyl)-6-[(4-methylpiperazin-1-yl)methyl]-3H-1,3-benzodiazol-2-ylidene]pyridine-4-carboxamide (60 mg, 0.087 mmol, 1 equiv) in TFA (3 mL) at room temperature. The resulting mixture was stirred for 10 min at room temperature. The resulting mixture was concentrated under reduced pressure. The crude product (40 mg) was purified by Prep-HPLC with the following conditions (Column: Xselect CSH C18 OBD Column 30*150 mm 5 μm, n; Mobile Phase A: Water(0.1I % FA), Mobile Phase B: ACN; Flow rate: 60 m/min; Gradient: 11% B to 28% B in 8 min, 28% B; Wave Length: 254/220 nm; RT1(min): 8; Number Of Runs: 0) to afford 2-(2-formyl-3-hydroxyphenoxymethyl)-N-[(2E)-1-(2-hydroxy-2-methylpropyl)-6-[(4-methylpiperazin-1-yl)methyl]-3H-1,3-benzodiazol-2-ylidene]pyridine-4-carboxamide (4.4 mg, 8.79%) as a yellow solid. MS (ESI) calcd. for C₃1H₃₆N_{605, 572.27} m/z, found: 573.27[M+H]⁺. ¹H NMR (300 MHz, DMSO-d⁶) δ (ppm): 12.89 (s, 1H), 11.74 (s, 1H), 10.48 (s, 1H), 8.73-8.75 (m, 1H), 8.15-8.20 (m, 1H), 8.00-8.02 (m, 1H), 7.46-7.56 (m, 3H), 7.15-7.18 (m, 1H), 6.67-6.70 (m, 1H), 6.55-6.58 (m, 1H), 5.44 (s, 2H), 4.94 (s, 1H), 4.18 (s, 2H), 3.34-3.53 (m, 2H), 2.37-2.50 (m, 8H), 2.18 (s, 3H), 1.17 (s, 6H).

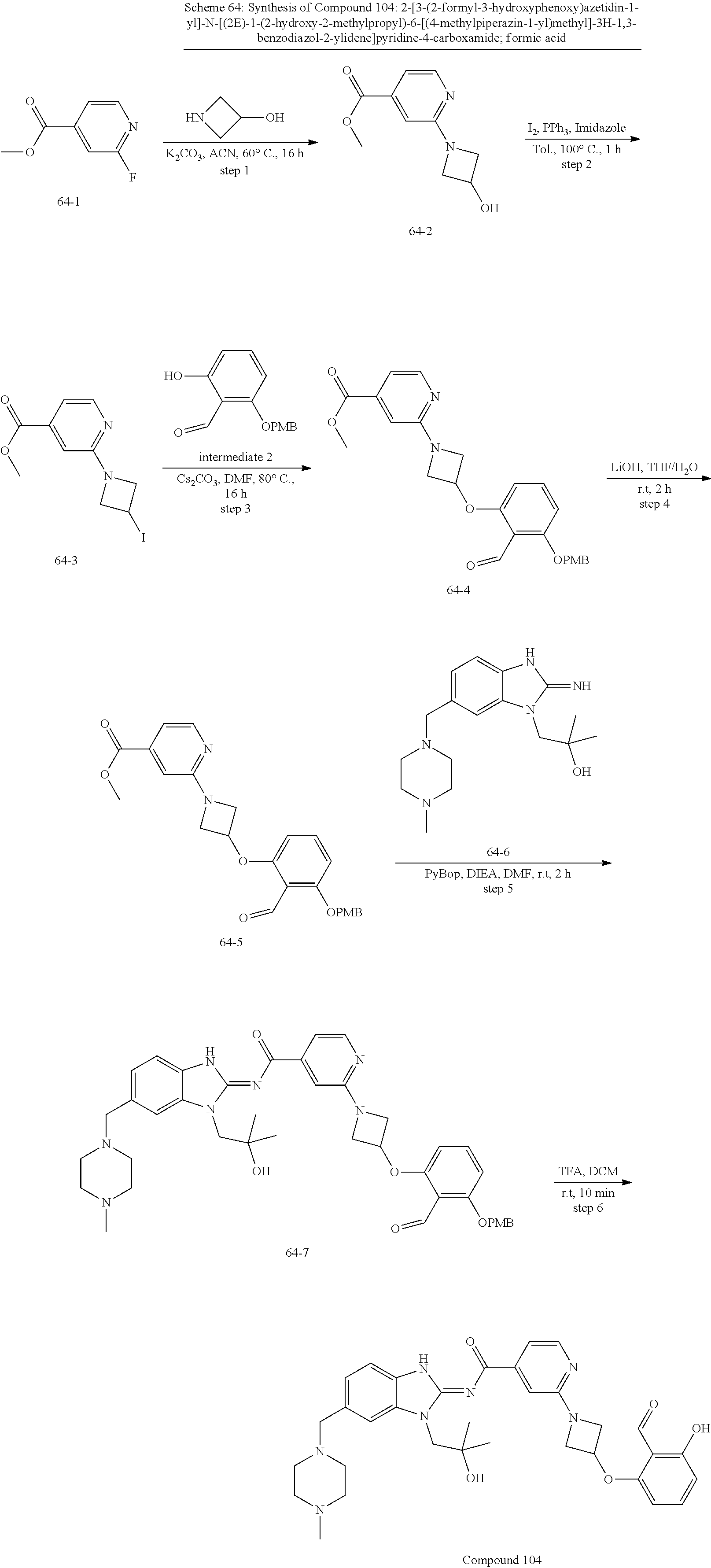

### Step 1: Synthesis of methyl 2-(3-hydroxyazetidin-1-yl)pyridine-4-carboxylate

A solution of methyl 2-fluoropyridine-4-carboxylate (1 g, 6.44 mmol, 1 equiv.), azetidin-3-ol (471.2 mg, 6.44 mmol, 1 equiv.) and K₂CO₃ (1.34 g, 9.67 mmol, 1.5 equiv.) in ACN (10 mL) was stirred for 16 h at 60° C. The desired product was detected by LCMS. The reaction was concentrated under reduced pressure. The residue was purified by silica gel column chromatography, eluted with EA/PE (0-80%) to afford methyl 2-(3-hydroxyazetidin-1-yl)pyridine-4-carboxylate (580 mg, 34.0%) as a yellow solid. MS (ESI) calcd. for C₁₀H₁₂N₂O₃, 208.08 m/z, found 209.05 [M+H]⁺.

### Step 2: Synthesis of methyl 2-(3-iodoazetidin-1-yl)pyridine-4-carboxylate

A solution of methyl 2-(3-hydroxyazetidin-1-yl)pyridine-4-carboxylate (580 mg, 2.79 mmol, 1 equiv.), PPh₃ (1.5 g, 5.57 mmol, 2 equiv.), imidazole (568.9 mg, 8.36 mmol, 3 equiv.) and I2 (1.1 g, 4.18 mmol, 1.5 equiv.) in toluene (5 mL) was stirred for 1 h at 100° C. under N₂ atmosphere. Desired product was detected by LCMS. The resulting mixture was concentrated under reduced pressure. The residue was purified by silica gel column chromatography, eluted with EA/PE (0-40%) to afford methyl 2-(3-{2-formyl-3-[(4-methoxyphenyl)methoxy]phenoxy}azetidin-1-yl)pyridine-4-carboxylate) (450 mg) as a white solid. MS (ESI) calcd. for C₁₀HiiIN₂O_{2-, 317.98} m/z, found 318.90 [M+H]⁺.

### Step 3: Synthesis of methyl 2-(3-{2-formyl-3-[(4-methoxyphenyl)methoxy]phenoxy}azetidin-1-yl)pyridine-4-carboxylate

A solution of methyl 2-(3-iodoazetidin-1-yl)pyridine-4-carboxylate (450 mg, 1.41 mmol, 1 equiv.), 2-hydroxy-6-[(4-methoxyphenyl)methoxy]benzaldehyde (730.7 mg, 2.83 mmol, 2 equiv.) and Cs₂CO₃ (691.3 mg, 2.12 mmol, 1.5 equiv.) in DMF (10 mL) was stirred for 16 h at 80° C. Desired product was detected by LCMS. The residue was purified by reverse flash chromatography with MeCN/H₂O (0.5% TFA) from 0%˜70% to afford methyl 2-(3-{2-formyl-3-[(4-methoxyphenyl)methoxy]phenoxy}azetidin-1-yl)pyridine-4-carboxylate (700 mg, 97.7%) as a white solid. MS (ESI) calcd. for C₂₅H₂₄N₂O₆, 448.16 m/z, found 449.10 [M+H]⁺.

### Step 4: Synthesis of 2-(3-{2-formyl-3-[(4-methoxyphenyl)methoxy]phenoxy}azetidin-1-yl)pyridine-4-carboxylic acid

A solution of methyl 2-(3-{2-formyl-3-[(4-methoxyphenyl)methoxy]phenoxy}azetidin-1-yl)pyridine-4-carboxylate (720 mg, 1.60 mmol, 1 equiv.) and LiOH (1.07 mL, 3.21 mmol, 2 equiv., 3M in H₂O) in THF (5 mL) and H₂O (1 mL) was stirred for 2 h at 25° C. Desired product was detected by LCMS. The mixture was neutralized to pH=6 with HCl (2 mol/L) and then concentrated under reduced pressure to afford 2-(3-{2-formyl-3-[(4-methoxyphenyl)methoxy]phenoxy}azetidin-1-yl)pyridine-4-carboxylic acid (700 mg, 92.97%) as a brown solid. MS (ESI) calcd. for C₂₄H₂₂N₂O₆, 434.14 m/z, found 435.10 [M+H].

### Step 5: Synthesis of 2-(3-{2-formyl-3-[(4-methoxyphenyl)methoxy]phenoxy}azetidin-1-yl)-N-[(2E)-1-(2-hydroxy-2-methylpropyl)-6-[(4-methylpiperazin-1-yl)methyl]-3H-1,3-benzodiazol-2-ylidene]pyridine-4-carboxamide

A solution of 2-(3-{2-formyl-3-[(4-methoxyphenyl)methoxy]phenoxy}azetidin-1-yl)pyridine-4-carboxylic acid (100 mg, 0.23 mmol, 1.2 equiv.), 1-{2-imino-6-[(4-methylpiperazin-1-yl)methyl]-3H-1,3-benzodiazol-1-yl}-2-methylpropan-2-ol (60.9 mg, 0.19 mmol, 1 equiv.), PyBOP (149.7 mg, 0.29 mmol, 1.5 equiv.) and DIEA (49.6 mg, 0.38 mmol, 2 equiv.) in DMF (3 mL) was stirred for 2 h at 25° C. Desired product was detected by LCMS.

The residue was purified by reverse flash chromatography with MeCN/H₂O (0.5% TFA) from 0% to 55% to afford 2-(3-{2-formyl-3-[(4-methoxyphenyl)methoxy]phenoxy}azetidin-1-yl)-N—[(2E)-1-(2-hydroxy-2-methylpropyl)-6-[(4-methylpiperazin-1-yl)methyl]-3H-1,3-benzodiazol-2-ylidene]pyridine-4-carboxamide (70 mg, 34.3%) as a yellow solid. MS (ESI) calcd. for C₄₁H₄₇N₇O₆, 733.35 m/z, found 734.45 [M+H]⁺.

### Step 6: Synthesis of 2-[3-(2-formyl-3-hydroxyphenoxy)azetidin-1-yl]-N-[(2E)-1-(2-hydroxy-2-methylpropyl)-6-[(4-methylpiperazin-1-yl)methyl]-3H-1,3-benzodiazol-2-ylidene]pyridine-4-carboxamide; formic acid

A solution of 2-(3-{2-formyl-3-[(4-methoxyphenyl)methoxy]phenoxy}azetidin-1-yl)-N-[(2E)-1-(2-hydroxy-2-methylpropyl)-6-[(4-methylpiperazin-1-yl)methyl]-3H-1,3-benzodiazol-2-ylidene]pyridine-4-carboxamide (30 mg, 0.04 mmol, 1 equiv.) in TFA (0.2 mL) and DCM (0.6 mL) was stirred for 10 min at rt. The desired product was detected by LCMS. The mixture was concentrated under reduced pressure. The crude product was purified by Prep-HPLC with the following conditions (Column: Sunfire prep C18 column, 30*150 mm, 5 μm; Mobile Phase A: Water(0.1% FA), Mobile Phase B: ACN; Flow rate: 60 mL/min; Gradient: 2% B to 24% B in 10 min, 24% B; Wave Length: 254/220 nm; RT1(min): 9.2; Number Of Runs: 0) to afford 2-[3-(2-formyl-3-hydroxyphenoxy)azetidin-1-yl]-N-[(2E)-1-(2-hydroxy-2-methylpropyl)-6-[(4-methylpiperazin-1-yl)methyl]-3H-1,3-benzodiazol-2-ylidene]pyridine-4-carboxamide (1.6 mg, 6.3%) as a white solid. MS (ESI) calcd. for C₃₄H₄₁N₇O₇, 613.30 m/z, found 614.25 [M+H]⁺. ¹H NMR (400 MHz, DMSO-d₆) δ (ppm): 12.81 (s, 1H), 11.78 (s, 1H), 10.38 (s, 1H), 8.22 (d, J=5.2 Hz, 1H), 7.60-7.51 (m, 2H), 7.48 (d, J=8.2 Hz, 1H), 7.38 (d, J=5.5 Hz, 1H), 7.21-7.11 (m, 2H), 6.59 (d, J=8.5 Hz, 1H), 6.46 (d, J=8.2 Hz, 1H), 5.34 (s, 1H), 4.99 (s, 1H), 4.56-4.47 (m, 2H), 4.20 (s, 2H), 4.11-4.04 (m, 3H), 3.57 (s, 5H), 2.54-2.47 (m, 3H), 2.31 (s, 3H), 2.08 (s, 1H), 1.23 (s, 6H).

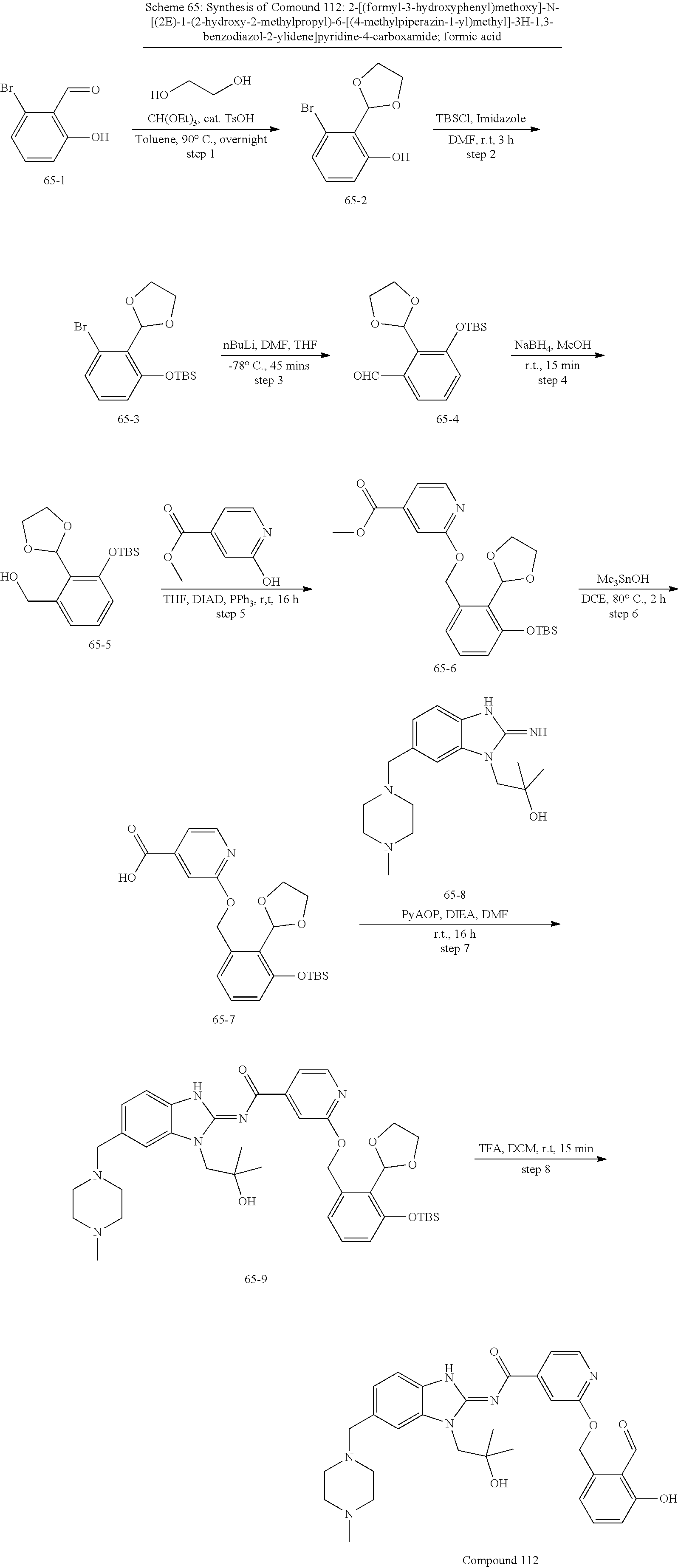

### Step 1: Synthesis of 3-bromo-2-(1,3-dioxolan-2-yl)phenol

To a stirred solution of 2-bromo-6-hydroxybenzaldehyde (14.6 g, 72.63 mmol, 1 equiv) and TsOH (1.25 g, 7.26 mmol, 0.1 equiv) in toluene (140 mL) was added triethyl orthoformate (48.4 g, 326.83 mmol, 4.5 equiv) and ethylene glycol (40.6 g, 653.67 mmol, 9 equiv) at 25° C.

The resulting mixture was stirred for overnight at 90° C. The mixture was concentrated in vacuum and purified by silica gel column with EA/PE (0-30%) to afford 7.27 g red oil (88.4% purity, 36.1% yiled). MS (ESI) calcd. for C₉H₉BrO₃, 243.97 m/z, found 244.97 [M+H]⁺.

### Step 2: Synthesis of 3-bromo-2-(1,3-dioxolan-2-yl)phenoxy(tert-butyl)dimethylsilane

To a stirred solution of 3-bromo-2-(1,3-dioxolan-2-yl)phenol (2.0 g, 8.16 mmol, 1 equiv) and imidazole (0.83 g, 12.24 mmol, 1.5 equiv) in DMF (20 mL) was added TBSCI (1.50 g, 9.79 mmol, 1.2 equiv) at 0° C. under N₂ atmosphere. The resulting mixture was stirred at 22° C. for additional 3 h. Desired product was detected by LCMS. The resulting mixture was diluted with EA (100 mL). The resulting mixture was extracted with water (50 mL×3). The combined organic layers were washed with brine (50 mL), dried over anhydrous Na₂SO₄. After filtration, the filtrate was concentrated under reduced pressure. The residue was purified by silica gel column chromatography, eluted with EtOAc/pet ether (0˜10%) to afford 3-bromo-2-(1,3-dioxolan-2-yl)phenoxy(tert-butyl)dimethylsilane (3 g, 93.1%) as a white solid. MS (ESI) calcd. for C₁₅H₂₃BrO₃Si, 359.33 m/z, found 360.33 [M+H]⁺.

### Step 3: Synthesis of 3-[(tert-butyldimethylsilyl)oxy]-2-(1,3-dioxolan-2-yl)benzaldehyde

A solution of 3-bromo-2-(1,3-dioxolan-2-yl)phenoxy(tert-butyl)dimethylsilane (3 g, 8.35 mmol, 1 equiv) in THF (15 mL) was added n-BuLi (4.0 mL, 10.02 mmol, 1.2 equiv) for 30 min at −78° C. under nitrogen atmosphere. The reaction mixture was stirred for 0.5 h, and DMF (5 mL) was added dropwise at −78° C. The resulting mixture was stirred for additional 15 min at −78° C. The reaction was quenched by the addition of NHCl₄ (30 mL) at −78° C. The resulting mixture was diluted with EA (100 mL). The resulting mixture was extracted with water (50 mL×3). The combined organic layers were washed with brine (100 mL), dried over anhydrous Na₂SO₄. After filtration, the filtrate was concentrated under reduced pressure. The residue was purified by silica gel column chromatography, eluted with EtOAc/pet (0˜40%) to afford 3-[(tert-butyldimethylsilyl)oxy]-2-(1,3-dioxolan-2-yl)benzaldehyde (2.5 g, 88.3%) as a yellow oil. MS (ESI) calcd. for C₁₆H₂₄O₄Si, 308.14 m/z, found 309.14 [M+H].

### Step 4: Synthesis of {3-[(tert-butyldimethylsilyl)oxy]-2-(1,3-dioxolan-2-yl)phenyl}methanol

To a stirred solution of 3-[(tert-butyldimethylsilyl)oxy]-2-(1,3-dioxolan-2-yl)benzaldehyde (3 g, 9.73 mmol, 1 equiv) in MeOH (10 mL) was added NaBH4 (0.18 g, 4.86 mmol, 0.5 equiv) slowly at 0° C. under N₂ atmosphere. Desired product was detected by LCMS. The resulting mixture was diluted with EA (100 mL). The resulting mixture was extracted with water (50 mL×3). The combined organic layers were washed with brine (50 mL), dried over anhydrous Na₂SO₄. After filtration, the filtrate was concentrated under reduced pressure. The residue was purified by silica gel column chromatography, eluted with EA/PE (0˜100%) to afford {3-[(tert-butyldimethylsilyl)oxy]-2-(1,3-dioxolan-2-yl)phenyl}methanol (2.5 g, 74.5%) as a colorless solid. MS (ESI) calcd. for C₁₆H₂₆O₄Si, 310.16 m/z, found 333.16 [M+Na]⁺.

### Step 5: Synthesis of methyl 2-({3-[(tert-butyldimethylsilyl)oxy]-2-(1,3-dioxolan-2-yl)phenyl}methoxy)pyridine-4-carboxylate

To a stirred solution of {3-[(tert-butyldimethylsilyl)oxy]-2-(1,3-dioxolan-2-yl)phenyl}methanol (1.5 g, 4.83 mmol, 1 equiv) and methyl 2-hydroxypyridine-4-carboxylate (0.89 g, 5.79 mmol, 1.2 equiv) and PPh₃ (1.90 g, 7.24 mmol, 1.5 equiv) in THF (10 mL) was added DIAD (1.47 g, 7.24 mmol, 1.5 equiv) dropwise at 0° C. under N₂ atmosphere. The resulting mixture was stirred at rt for additional 16 h. The resulting mixture was diluted with EA (50 mL). The resulting mixture was extracted with water (50 mL×3). The combined organic layers were washed with brine (50 mL), dried over anhydrous Na₂SO₄. After filtration, the filtrate was concentrated under reduced pressure. The residue was purified by silica gel column chromatography, eluted with EA/PE (0˜100%) to afford methyl 2-({3-[(tert-butyldimethylsilyl)oxy]-2-(1,3-dioxolan-2-yl)phenyl}methoxy)pyridine-4-carboxylate (200 mg, 6.5%) as a white solid. MS (ESI) calcd. for C₂₃H₃₁NO₆Si, 445.19 m/z, found 446.19 [M+H].

### Step 6: Synthesis of 2-({3-[(tert-butyldimethylsilyl)oxy]-2-(1,3-dioxolan-2-yl)phenyl}methoxy)pyridine-4-carboxylic acid

A solution of methyl 2-({3-[(tert-butyldimethylsilyl)oxy]-2-(1,3-dioxolan-2-yl)phenyl}methoxy)pyridine-4-carboxylate (160 mg, 0.36 mmol, 1 equiv) and trimethyltin hydroxide (194.8 mg, 1.08 mmol, 3 equiv) in DCE (3 mL) was stirred for 2 h at 80° C. under N2 atmosphere. The residue was purified by Prep-TLC (MeOH/DCM 0˜30%) to afford 2-({3-[(tert-butyldimethylsilyl)oxy]-2-(1,3-dioxolan-2-yl)phenyl}methoxy)pyridine-4-carboxylic acid (80 mg, 30.1%) as a white solid. MS (ESI) calcd. for C₂₂H₂₉NO₆Si, 431.17 m/z, found 432.17 [M+H]⁺.

### Step 7: Synthesis of 2-({3-[(tert-butyldimethylsilyl)oxy]-2-(1,3-dioxolan-2-yl)phenyl}methoxy)-N-[(2E)-1-(2-hydroxy-2-methylpropyl)-6-[(4-methylpiperazin-1-yl)methyl]-3H-1,3-benzodiazol-2-ylidene]pyridine-4-carboxamide

To a stirred solution of 2-({3-[(tert-butyldimethylsilyl)oxy]-2-(1,3-dioxolan-2-yl)phenyl}methoxy)pyridine-4-carboxylic acid (70 mg, 0.16 mmol, 1 equiv), 1-{2-imino-6-[(4-methylpiperazin-1-yl)methyl]-3H-1,3-benzodiazol-1-yl}-2-methylpropan-2-ol (61.8 mg, 0.19 mmol, 1.2 equiv) and PyAOP (84.6 mg, 0.16 mmol, 1.0 equiv) in DMF was added DIEA (62.9 mg, 0.49 mmol, 3 equiv) dropwise at 22° C. under N₂ atmosphere. The resulting mixture was stirred at r.t for additional 16 h. The resulting mixture was diluted with EA (20 mL). The resulting mixture was extracted with water (20 mL×3). The combined organic layers were washed with brine (10 mL), dried over anhydrous Na₂SO₄. After filtration, the filtrate was concentrated under reduced pressure. The residue was purified by Prep-TLC (MeOH/DCM 0˜20%) to afford 2-({3-[(tert-butyldimethylsilyl)oxy]-2-(1,3-dioxolan-2-yl)phenyl}methoxy)-N-[(2E)-1-(2-hydroxy-2-methylpropyl)-6-[(4-methylpiperazin-1-yl)methyl]-3H-1,3-benzodiazol-2-ylidene]pyridine-4-carboxamide (20 mg, 10.1%) as a white solid. MS (ESI) calcd. for C₃₉H₅₄N₆O₆Si, 730.98 m/z, found 731.98 [M+H]⁺.

### Step 8: Synthesis of 2-[(2-formyl-3-hydroxyphenyl)methoxy]-N-[(2E)-1-(2-hydroxy-2-methylpropyl)-6-[(4-methylpiperazin-1-yl)methyl]-3H-1,3-benzodiazol-2-ylidene]pyridine-4-carboxamide; formic acid

A solution of 2-{[2-(1,3-dioxolan-2-yl)-3-(methoxymethoxy)phenyl]methoxy}-N-[(2E)-1-(2-hydroxy-2-methylpropyl)-6-[(4-methylpiperazin-1-yl)methyl]-3H-1,3-benzodiazol-2-ylidene]pyridine-4-carboxamide (20 mg, 0.03 mmol, 1 equiv) and TFA (0.2 mL) in DCM (1 mL) was stirred for 15 min at rt. The desired product was detected by LCMS. The mixture was purified by Prep-HPLC under the condition: Column: Xselect CSH C18 OBD Column 30*150 mm 5 μm, n; Mobile Phase A: Water(0.1I % FA), Mobile Phase B: ACN; Flow rate: 60 m/min; Gradient: 5% B to 25% B in 9 min, 25% B; Wave Length: 254/220 nm; RT1(min): 9; Number Of Runs: 0 to afford 2-[(2-formyl-3-hydroxyphenyl)methoxy]-N-[(2E)-1-(2-hydroxy-2-methylpropyl)-6-[(4-methylpiperazin-1-yl)methyl]-3H-1,3-benzodiazol-2-ylidene]pyridine-4-carboxamide (2.5 mg, 13.9%) as a white solid. MS (ESI) calcd. for C₃₁H₃₆N₆O₇, 572.27 m/z, found 573.28 [M+H]⁺. ¹H NMR (400 MHz, DMSO-d₆) δ 12.89 (s, 1H), 11.09 (s, 1H), 10.58 (s, 1H), 7.76 (d, J=7.0 Hz, 1H), 7.57 (s, 1H), 7.49 (d, J=8.2 Hz, 1H), 7.43 (t, J=8.0 Hz, 1H), 7.18 (d, J=11.0 Hz, 2H), 6.94 (t, J=7.8 Hz, 2H), 6.27 (d, J=7.8 Hz, 1H), 5.45 (s, 2H), 4.97 (s, 1H), 4.19 (s, 2H), 3.56 (s, 2H), 2.50-2.35 (m, 6H), 2.32-2.25 (m, 5H), 1.28-1.20 (m, 6H).

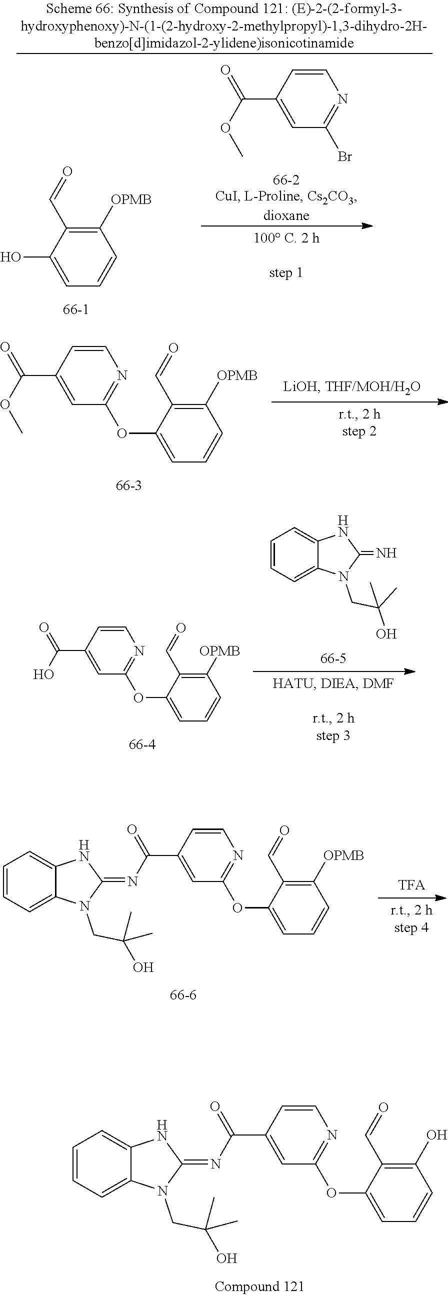

### Step 1: Synthesis of methyl 2-(2-formyl-3-((4-methoxybenzyl)oxy)phenoxy)isonicotinate

A solution of 2-hydroxy-6-[(4-methoxyphenyl)methoxy]benzaldehyde (300 mg, 1.162 mmol, 1 equiv) methyl 2-bromopyridine-4-carboxylate (301.12 mg, 1.394 mmol, 1.2 equiv) CuI (22.12 mg, 0.116 mmol, 0.1 equiv) Cs₂CO₃ (756.92 mg, 2.324 mmol, 2 equiv) in 1,4-dioxane (2 mL, 6.67 equiv), followed by the addition of L-proline (26.75 mg, 0.232 mmol, 0.2 equiv). The resulting mixture was stirred for 2 h at 110° C. under N₂ atmosphere. The crude product (100 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 8% B to 29% B in 8 min, 29% B; Wave Length: 254/220 nm; RT1(min): 8; Number Of Runs: 0) to afford methyl 2-{2-formyl-3-[(4-methoxyphenyl)methoxy]phenoxy}pyridine-4-carboxylate (200 mg, 43.77%) as a yellow solid.

### Step 2: Synthesis of 2-(2-formyl-3-((4-methoxybenzyl)oxy)phenoxy)isonicotinic acid

Into a 15 ml microwave tube were added methyl 2-{2-formyl-3-[(4-methoxyphenyl)methoxy]phenoxy}pyridine-4-carboxylate (200 mg, 0.508 mmol, 1 equiv) lithiumol (36.53 mg, 1.524 mmol, 3 equiv) and THF (1 mL) H₂O (1 mL) MeOH (1 mL) at rt for 1h. The resulting mixture was extracted with EA (10 ml×3) and dried over anhydrous Na₂SO₄. After filtration, the filtrate was concentrated under reduced pressure. The combined organic layers were washed with EA (10 ml×3). After filtration, the filtrate was concentrated under reduced pressure to afford 2-{2-formyl-3-[(4-methoxyphenyl)methoxy]phenoxy}pyridine-4-carboxylic acid (100 mg, 51.85%) as a yellow solid. LCMS:379.11[M+H]⁺.

### Step 3: Synthesis of (E)-2-(2-formyl-3-((4-methoxybenzyl)oxy)phenoxy)-N-(1-(2-hydroxy-2-methylpropyl)-1,3-dihydro-2H-benzo[d]imidazol-2-ylidene)isonicotinamide

A solution of 2-{2-formyl-3-[(4-methoxyphenyl)methoxy]phenoxy}pyridine-4-carboxylic acid (100 mg, 0.264 mmol, 1 equiv), 1-(2-imino-3H-1,3-benzodiazol-1-yl)-2-methylpropan-2-ol (64.93 mg, 0.317 mmol, 1.2 equiv), HATU (120.27 mg, 0.317 mmol, 1.2 equiv) in DMF (2 mL, 20.00 equiv), followed by the addition of DIEA (68.14 mg, 0.528 mmol, 2 equiv), The resulting mixture was stirred for 10 min at room temperature. The crude product (100 mg) was purified by Prep-HPLC with the following conditions (Column: XSelect CSH Fluoro Phenyl, 30*150 mm, 5 μm; Mobile Phase A: Water(0.1% FA), Mobile Phase B: ACN; Flow rate: 60 mL/min; Gradient: 8% B to 26% B in 10.5 min; Wave Length: 220 nm; RT1(min): 8.8; Number Of Runs: 5) to afford 2-{2-formyl-3-[(4-methoxyphenyl)methoxy]phenoxy}-N-[(2E)-1-(2-hydroxy-2-methylpropyl)-3H-1,3-benzodiazol-2-ylidene]pyridine-4-carboxamide (15 mg, 10.04%) as a yellow solid. LCMS:566.22[M+H]⁺.

### Step 4: Synthesis of (E)-2-(2-formyl-3-hydroxyphenoxy)-N-(1-(2-hydroxy-2-methylpropyl)-1,3-dihydro-2H-benzo[d]imidazol-2-ylidene)isonicotinamide

To a stirred solution of 2-{2-formyl-3-[(4-methoxyphenyl)methoxy]phenoxy}-N-[(2E)-1-(2-hydroxy-2-methylpropyl)-3H-1,3-benzodiazol-2-ylidene]pyridine-4-carboxamide (15 mg, 0.026 mmol, 1 equiv) in TFA (1 mL, 66.67 equiv) at room temperature. The resulting mixture was stirred for 10 min at room temperature.The resulting mixture was concentrated under reduced pressure. The crude product (100 mg) was purified by Prep-HPLC with the following conditions (Column: YMC-Actus Triart C18 ExR5, 30*150 mm, 5 μm; Mobile Phase A: Water(10 mmol/L NH₄HCO₃), Mobile Phase B: ACN; Flow rate: 60 mL/min; Gradient: 15% B to 30% B in 10.5 min; Wave Length: 572; 220 nm; RTT(min): 10.5; Number Of Runs: 4) to afford 2-(2-formyl-3-hydroxyphenoxy)-N-[(2E)-1-(2-hydroxy-2-methylpropyl)-3H-1,3-benzodiazol-2-ylidene]pyridine-4-carboxamide (1.1 mg, 9.17%) as a yellow solid. MS (ESI) calcd. for C₂₄H₂₂N₄O₅, 446.16 m/z, found 447.17 [M+H]⁺. ¹H NMR (300 MHz, DMSO-d₆) δ (ppm): 12.94-12.96 (m, 1H), 11.32 (s, 1H), 10.23 (s, 1H), 8.26-8.28 (m, 1H), 7.81-7.83 (m, 1H), 7.59-7.69 (m, 4H), 7.26-7.28 (m, 2H), 6.86-6.89 (m, 1H), 6.69-6.72 (m, 1H), 4.20-4.30 (m, 2H), 1.21 (s, 6H).

The following compounds were synthesized according to the preceding method or analogous methods thereto:

| Cmpd | |
|---|---|
| No. | Characterization Data |
| 124 | MS (ESI) calcd. for C₂₄H₂₂N₄O₄, 430.16 m/z, found: 431.15 [M + H]⁺.¹H NMR |
| | (400 MHZ, DMSO-d₆) δ ppm: 15.65 (s, 1H), 12.96 (s, 1H), 10.50 (s, 1H), 8.83 |
| | (s, 2H), 8.37-8.40 (m, 1H), 8.15-8.16 (m, 1H), 7.78-7.80 (m, 1H), 7.58- |
| | 7.66 (m, 2H), 7.26-7.28 (m, 2H), 7.13-7.15 (m, 1H), 4.97 (s, 1H), 4.29 (s, |
| | 2H), 1.27 (s, 6H) |
| 127 | MS (ESI) calcd. for C₂₆H₂₂N₄O₄, 454.16 m/z, found: 455.10 [M + H]⁺. ¹H NMR |
| | (400 MHZ, DMSO-d₆) δ ppm: 12.93 (s, 1H), 11.35 (s, 1H), 10.54 (s, 1H), 8.78- |
| | 8.80 (m, 1H), 8.34 (s, 1H), 8.09-8.11 (m, 1H), 7.57-7.68 (m, 3H), 7.22- |
| | 7.31 (m, 3H), 7.11-7.13 (m, 1H), 4.92 (s, 1H), 4.26 (s, 2H), 1.26 (s, 6H) |
| 128 | MS (ESI) calcd. for C₂₆H₂₂N₄O₄, 454.16 m/z, found: 455.10 [M + H]⁺. ¹H NMR |
| | (400 MHZ, DMSO-d₆) δ ppm: 12.93 (s, 1H), 11.35 (s, 1H), 10.54 (s, 1H), 8.78- |
| | 8.80 (m, 1H), 8.34 (s, 1H), 8.09-8.11 (m, 1H), 7.57-7.68 (m, 3H), 7.22- |
| | 7.31 (m, 3H), 7.11-7.13 (m, 1H), 4.92 (s, 1H), 4.26 (s, 2H), 1.26 (s, 6H) |
| 129 | MS (ESI) calcd. for C₂₅H₂₄N₄O₅, 460.17 m/z, found: 461.20 [M + H]⁺. ¹H NMR |
| | (300 MHz, DMSO-d₆) δ (ppm): 12.90 (s, 1H), 11.37 (s, 1H), 10.24 (s, 1H), |
| | 7.59-7.69 (m, 2H), 7.51-7.58 (m, 2H), 7.42-7.48 (s, 1H), 7.21-7.26 (m, |
| | 2H), 6.80-6.87 (m, 1H), 6.62-6.69 (m, 1H), 4.20 (s, 2H), 2.50 (s, 3H), 1.20 |
| | (s, 6H). |
| 130 | MS (ESI) calcd. for C₃₀H₂₆N₄O₄, 506.20 m/z, found 507.15 [M + H]⁺. ¹H NMR |
| | (300 MHz, DMSO-d₆) δ (ppm): 12.93 (s, 1H), 11.67 (s, 1H), 9.93 (s, 1H), 8.81- |
| | 8.89 (m, 1H), 8.61-8.67 (m, 1H), 8.17-8.27 (m, 1H), 8.09-8.16 (m, 1H), |
| | 8.00-8.08 (m, 1H), 7.62-7.74 (m, 3H), 7.52-7.62 (m, 2H), 7.19-7.32 (m, |
| | 2H), 7.01-7.12 (m, 2H), 4.26 (s, 2H), 3.93-4.05 (m, 1H), 1.23 (s, 6H) |
| 131 | MS (ESI) calcd. for C₂₈H₃₀N₆O₄, 514.58 m/z, found: 515.15 [M + H]⁺. ¹H NMR |
| | (400 MHZ, DMSO-d₆) δ (ppm): 12.85 (s, 1H), 11.67 (s, 1H), 10.27 (s, 1H), |
| | 8.27-8.28 (m, 1H), 7.63-7.65 (m, 1H), 7.50-7.55 (m, 3H), 7.38-7.40 (m, |
| | 1H), 7.21-7.27 (m, 2H), 6.62-6.71 (m, 2H), 5.03 (s, 1H), 4.23 (s, 2H), 3.76 |
| | (s, 4H), 3.15-3.20 (m, 4H), 1.23 (s, 6H) |
| 132 | MS (ESI) calcd. for C₃₀H₂₆N₄O₄: 506.19 m/z, found: 507.15 [M + H]⁺. ¹H NMR |
| | (400 MHZ, DMSO-d₆) δ ppm: 12.96 (s, 1H), 11.69 (s, 1H), 9.94 (s, 1H), 8.86- |
| | 8.73 (m, 1H), 8.61 (s, 1H), 8.22-8.24 (m, 2H), 8.07-8.08 (m, 1H), 7.57-7.69 (m, |
| | 5H), 7.23-7.29 (m, 2H), 7.01-7.07 (m, 2H), 5.01 (s, 1H), 4.28 (s, 2H), 1.23-1.67 |
| | (m, 6H) |
| 133 | MS (ESI) calcd. for C₂₅H₂₄N₄O₅, 460.17 m/z, found: 461.15 [M + H]⁺. ¹H NMR |
| | (400 MHZ, DMSO-d₆) δ ppm: 12.95 (s, 1H), 11.74 (s, 1H), 10.49 (s, 1H), 8.74- |
| | 8.75 (m, 1H), 8.22 (s, 1H), 8.01-8.03 (m, 1H), 7.64-7.66 (m, 1H), 7.50- |
| | 7.56 (m, 2H), 7.21-7.28 (m, 2H), 6.69 (d, J = 8.4, 1H), 6.57 (d, J = 8.4, 1H), |
| | 5.45 (s, 2H), 4.96 (s, 1H), 4.20 (s, 2H), 1.18 (s, 6H) |
| 134 | MS (ESI) calcd. for C₂₄H₂₃N₅O₄: 445.18 m/z, found: 446.10 [M + H]⁺. ¹H NMR |
| | (300 MHz, DMSO-d₆) 8 (ppm): 10.22 (s, 1H), 8.24-8.36 (m, 1H), 8.02-8.05 |
| | (m, 1H), 7.72-7.74 (m, 1H), 7.63-7.66 (m, 1H), 7.55-7.58 (m, 1H), 7.40- |
| | 7.46 (m, 2H), 7.24-7.31 (m, 2H), 6.94-6.99 (m, 1H), 4.24 (s, 2H), 1.17-1.24 |
| | (m, 6H) |
| 135 | MS (ESI) calcd. for C₂₆H₂₆N₄O₄, 458 m/z, found: 459.20 [M + H]⁺. ¹H NMR |
| | (400 MHZ, DMSO-d₆) δ ppm: 10.45 (s, 1H), 8.63-8.64 (m, 1H), 7.87-7.90 |
| | (m, 2H), 7.63-7.65 (m, 1H), 7.54-7.56 (m, 1H), 7.38-7.42 (m, 1H), 7.20- |
| | 7.27 (m, 2H), 6.82-6.84 (m, 1H), 6.75-6.77 (m, 1H), 4.93 (s, 1H), 4.22 (s, |
| | 2H), 3.34-3.38 (m, 2H), 3.08-3.12 (m, 2H), 1.23 (s, 6H) |
| 136 | MS (ESI) calcd. for C₃₀H₂₆N₄O₄, 506.20 m/z, found: 507.20 [M + H]⁺. ¹H NMR |
| | (400 MHZ, DMSO-d₆) δ ppm: 12.91-12.97 (m, 1H), 10.96 (s, 1H), 10.36 (s, |
| | 1H), 8.84-8.86 (m, 1H), 8.58-8.61 (m, 1H), 8.20-8.23 (m, 2H), 7.97-8.06 |
| | (m, 3H), 7.83-7.86 (m, 2H), 7.58-7.68 (m, 2H), 7.26-7.28 (m, 2H), 7.14- |
| | 7.17 (m, 1H), 3.95-4.35 (m, 2H), 1.29-1.39 (m, 6H) |
| 137 | MS (ESI) calcd. for C₂₆H₂₂N₄O₄: 454.16 m/z, found: 455.10 [M + H]⁺. ¹H NMR |
| | (300 MHz, DMSO-d6) δ ppm: 12.94 (s, 1H), 11.33 (s, 1H), 10.17 (s, 1H), 8.76- |
| | 8.78 (m, 1H), 8.26 (s, 1H), 8.08-8.10 (m, 1H), 7.84-7.90 (m, 2H), 7.64- |
| | 7.66 (m, 1H), 7.56-7.59 (m, 1H), 7.24-7.29 (m, 2H), 7.11-7.16 (m, 1H), |
| | 4.94 (s, 1H), 4.25 (s, 2H), 1.25 (s, 6H) |
| 139 | MS (ESI) calcd. for C24H22N4O5, 446.16 m/z, found: 447.10 [M + H]⁺. ¹H |
| | NMR (400 MHZ, DMSO-d₆) δ ppm: 12.94-13.02 (m, 1H), 11.08 (s, 1H), |
| | 10.31 (s, 1H), 7.55-7.74 (m, 5H), 7.22-7.29 (m, 2H), 7.11-7.17 (m, 2H), |
| | 6.92-6.95 (m, 1H), 5.69-5.92 (m, 1H), 4.21 (s, 2H), 1.23 (s, 6H) |
| 140 | MS (ESI) calcd. for C24H22N4O5, 446.16 m/z, found: 447.10 [M + H]⁺. ¹H |
| | NMR (400 MHZ, DMSO-d₆) δ ppm: 12.95 (s, 1H), 10.67 (s, 1H), 10.26 (s, 1H), |
| | 8.19-8.25 (m, 1H), 7.77-7.78 (m, 1H), 7.49-7.70 (m, 4H), 7.03-7.35 (m, |
| | 4H), 4.95 (s, 1H), 4.19 (s, 2H), 1.21 (s, 6H) |

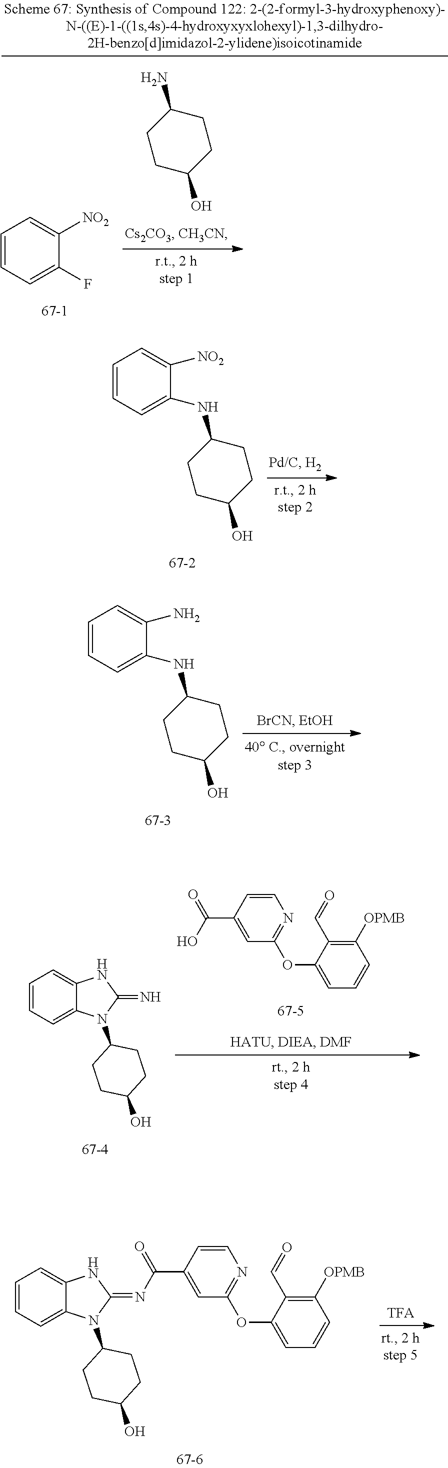

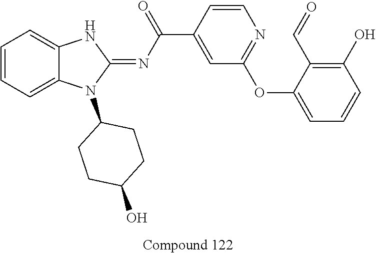 [SMILES: O=Cc1c(O)cccc1Oc1cc(C(=O)/N=c2\[nH]c3ccccc3n2[C@H]2CC[C@@H](O)CC2)ccn1]

### Step 1: Synthesis of (1s, 4s)-4-((2-nitrophenyl) amino) cyclohexan-1-ol

To a 100 ml round-bottomed flask equipped with a stirring bar were added 1-fluoro-2-nitrobenzene (500 mg, 3.544 mmol) and 20 mL DMF, followed by addition of (1s, 4s)-4-aminocyclohexan-1-ol (410 mg, 3.560 mmol) and Cs₂CO₃ (1.7 g, 5.202 mmol) at 0° C. The resulting solution was stirred at 60° C. for 2 h. The reaction progress was monitored by LCMS, and it showed the reaction was completed. The resulting mixture was extracted with EA (3×50 mL), The combined organic layers were washed with water (20 mL×3), dried over anhydrous Na₂SO₄. After filtration, the filtrate was concentrated under reduced pressure. The residue was concentrated to dryness in vacuum to give a yellow oil. The yellow oil was purified by silica gel chromatography (0-20% EA/PE) to afford the (1s, 4s)-4-((2-nitrophenyl) amino) cyclohexan-1-ol as a yellow solid (300 mg, 35.83% yield). MS (ESI) clad. for C₁₂H₁₆N₂O₃, 236.12 m/z, found 237.10 [M+H]⁺.

### Step 2: Synthesis of (1s, 4s)-4-((2-aminophenyl) amino) cyclohexan-1-ol

To a 50 ml round-bottomed flask equipped with a stirring bar was added (1s, 4s)-4-((2-nitrophenyl) amino) cyclohexan-1-ol (300 mg, 1.270 mmol) and MeOH (10 mL), followed by addition of PdOH/C (100 mg, 0.094 mmol). The mixture was stirred at rt for 12 h under a H₂ (g) (3.5 atm) atmosphere. Organic phase was obtained by filtration and concentrated to dryness to give a yellow solid. The yellow solid was recrystallized with PE (5 mL) to obtain (1s, 4s)-4-((2-aminophenyl) amino) cyclohexan-1-ol a yellow solid (200 mg, 76.36% yield). MS (ESI) calcd. for C₁₂H₁₈N₂O_{, 206.14} m/z, found 207.10 [M+H]*.

### Step 3: Synthesis of (1s, 4s)-4-(2-imino-2,3-dihydro-1H-benzo[d]imidazol-1-yl) cyclohexan-1-ol

(1s, 4s)-4-((2-aminophenyl) amino) cyclohexan-1-ol (200 mg, 0.970 mmol, 1 equip), a stir bar, ethyl alcohol (5 mL) and cyanogen bromide (150 mg, 1.416 mmol, 1.46 equip) were added to a 25-mL round-bottom flask and stirred until homogeneous. The resulting mixture was stirred at rt for 10 min. The residue was concentrated to dryness in vacuum to give a nearly yellow viscous oil. The yellow solid was purified by silica gel chromatography (0-10% Me OH/DCM) to afford the (1s, 4s)-4-(2-imino-2,3-dihydro-1H-benzo[d]imidazol-1-yl) cyclohexan-1-ol as a yellow solid (100.0 mg, 95.09% yield). MS (ESI) calcd. for C₁₃H₁₇N₃O_{, 231.14} m/z, found 232.15 [M+H]⁺.

### Step 4: Synthesis of 2-(2-formyl-3-((4-methoxybenzyl) oxy) phenoxy)-N—((E)-1-((1s, 4s)-4-hydroxycyclohexyl)-1,3-dihydro-2H-benzo[d]imidazol-2-ylidene) isonicotinamide

1-(2-morpholinoethyl)-1,3-dihydro-2H-benzo[d]imidazol-2-imine (100 mg, 0.406 mmol) and 5 mL DMF, followed by addition of 2-(2-formyl-3-((4-methoxybenzyl) oxy) phenyl) is nicotinic acid (153 mg, 0.403 mmol), DIEA (156 mg, 1.207 mmol) and HATU (160 mg, 0.421 mmol). The resulting solution was stirred at rt for 0.5 h. The resulting mixture was extracted with EA (3×10 mL). The combined organic layers were washed with water (3×10 mL), dried over anhydrous Na₂SO₄. After filtration, the filtrate was concentrated under reduced pressure. It to afford (E)-2-(2-formyl-3-((4-methoxybenzyl) oxy) phenoxy)-N-(1-(2-morpholinoethyl)-1,3-dihydro-2H-benzo[d]imidazol-2-ylidene) is nicotinamide as a yellow solid. (100 mg, 40.53% yield). MS (ESI) calcd. for C₃₄H₃₃N₅O₆, 607.24 m/z, found 608.50 [M+H]⁺.

### Step 5: Synthesis of 2-(2-formyl-3-hydroxyphenoxy)-N—((E)-1-((1s, 4s)-4-hydroxycyclohexyl)-1,3-dihydro-2H-benzo[d]imidazol-2-ylidene) isonicotinamide

2-(2-formyl-3-((4-methoxybenzyl) oxy) phenoxy)-N—((E)-1-((1s, 4s)-4-hydroxycyclohexyl)-1,3-dihydro-2H-benzo[d]imidazol-2-ylidene) is nicotinamide (100 mg, 0.169 mmol, 1 equip), a stir bar, trifluoroacetic acid (1 mL) were added to a 25-mL round-bottom flask and stirred until homogeneous. The resulting mixture was stirred at rt for 10 min. The residue was concentrated to dryness in vacuo to give a nearly yellow viscous oil. The crude product was then purified by Prep-HPLC with the following conditions: Column: XBridge Prep OBD C18 Column, 30*150 mm, 5 μm; Flow rate: 60 mL/min; Gradient: 35% B to 51% B in 10 mn, 51% B; Wave Length: 254/220 nm; RT1(min): 11.7; Number of Runs: 0. After lyophilization, it was afforded 2-(2-formyl-3-hydroxyphenoxy)-N—((E)-1-((1s, 4s)-4-hydroxycyclohexyl)-1,3-dihydro-2H-benzo[d]imidazol-2-ylidene) is nicotinamide (26.2 mg, 31.40%) as a yellow solid. MS (ESI) calcd. for C₂₆H₂₄N₄O₅, 472.17 m/z, found 473.20 [M+H].H NMR (300 MHz, DMSO-d6) δ (ppm): 12.94 (s, 1H), 11.34 (s, 1H), 10.24 (s, 1H), 8.21-8.30 (m, 1H), 8.88-8.97 (m, 1H), 7.78 (s, 1H), 7.56-7.73 (m, 3H), 7.20-7.40 (m, 2H), 6.85-6.95 (m, 1H), 6.70-6.80 (m, 1H), 4.84 (s, 1H), 4.67 (s, 1H), 3.98 (s, 1H), 2.70-2.90 (m, 2H), 1.80-1.99 (m, 2H), 1.45-1.78 (m, 4H).

The following compound(s) were synthesized according to the preceding method or analogous methods thereto:

| Cmpd No. | Characterization Data |
|---|---|
| 123 | MS (ESI) calcd. for C₂₆H₂₅N₅O₅, 487.19 m/z, found 488.10 [M + H]⁺. ¹H NMR |
| | (300 MHz, DMSO-d₆) δ (ppm): 12.91 (s, 1H), 11.31 (s, 1H), 10.24 (s, 1H), |
| | 8.21-8.30 (m, 1H), 7.80-7.90 (m, 1H), 7.72 (s, 1H), 7.51-7.66 (m, 3H), 7.21- |
| | 7.36 (m, 2H), 6.85-6.95 (m, 1H), 6.65-6.75 (m, 1H), 4.44 (s, 2H), 3.35- |
| | 3.55 (m, 4H), 2.78 (s, 2H), 2.55-2.65 (m, 2H), 2.33-2.43 (m, 2H). |

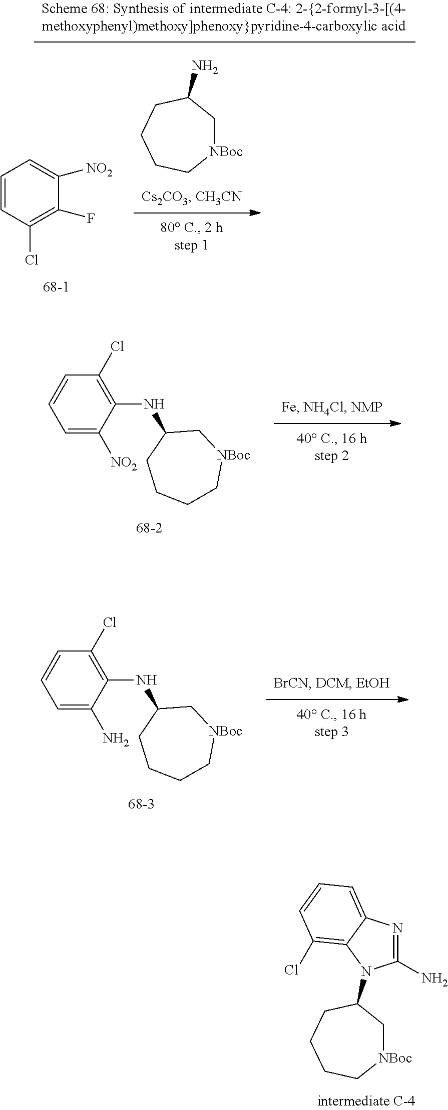 [SMILES: O=[N+]([O-])c1cccc(Cl)c1F ; CC(C)(C)OC(=O)N1CCCC[C@@H](N)C1 ; CC(C)(C)OC(=O)N1CCCC[C@@H](Nc2c(Cl)cccc2[N+](=O)[O-])C1 ; CC(C)(C)OC(=O)N1CCCC[C@@H](Nc2c(N)cccc2Cl)C1 ; CC(C)(C)OC(=O)N1CCCC[C@@H](n2c(N)nc3cccc(Cl)c32)C1]

### Step 1: Synthesis of tert-butyl (3R)-3-[(2-chloro-6-nitrophenyl)amino]azepane-1-carboxylate

To a stirred mixture of 1-chloro-2-fluoro-3-nitrobenzene (3 g, 17.09 mmol, 1 equiv) in CH₃CN (20 mL, 190.24 mmol) was added Cs₂CO₃ (16.7 g, 51.27 mmol, 3 equiv) at 25° C. The mixture was stirred for 2 h at 80° C. The desired product was detected by LCMS. The resulting mixture was concentrated under reduced pressure. The residue was purified by silica gel column chromatography, eluted with EA:PE (1:8) to afford tert-butyl (3R)-3-[(2-chloro-6-nitrophenyl)amino]azepane-1-carboxylate (6.3 g, 97.6%) as a red oil. MS (ESI) calcd. for C₁₇H₂₄ClN₃O₄, 369.1Om/z, found 392.10 [M+Na]+

### Step 2: Synthesis of tert-butyl (3R) 3-[(2-amino-6-chlorophenyl)amino]azepane-1-carboxylate

A mixture of tert-butyl (3R)-3-[(2-chloro-6-nitrophenyl)amino]azepane-1-carboxylate (6.3 g, 17.03 mmol, 1 equiv) and Fe (4.8 g, 85.17 mmol, 5 equiv) and NH₄C₁ (1.8 g, 34.06 mmol, 2 equiv) in NMP (25 mL) and H₂O (5 mL) was stirred for 16 h at 40° C. The desired product was detected by LCMS. The resulting mixture was filtered, and the filter cake was washed with EA (2×10 mL). The resulting organic was washed with H₂O (3×20 mL). The combined organic layers were dried over anhydrous Na₂SO₄. After filtration, the filtrate was concentrated under reduced pressure. The residue was purified by silica gel column chromatography, eluted with EA:PE (3:1) to afford tert-butyl-(3R)-3-[(2-amino-6-chlorophenyl)amino]azepane-1-carboxylate (5 g, 83.9%) as a red oil. MS (ESI) calcd. for C₁₇H₂₆ClN₃O_{2,339.1}Om/z, found 340.10 [M+H]+

### Step 3: Synthesis of tert-butyl (3R)-3-(2-amino-7-chloro-1,3-benzodiazol-1-yl)azepane-1-carboxylate

A solution of tert-butyl (3R)-3-[(2-amino-6-chlorophenyl)amino]azepane-1-carboxylate (5 g, 14.71 mmol, 1 equiv) in EtOH(10 mL) was treated with BrCN (1.9 g, 17.65 mmol, 1.2 equiv) in DCM (10 mL) dropwise at 0° C. The resulting mixture was stirred for 16 h at 40° C. Desired product was detected by LCMS. The reaction was quenched by the addition of H₂O (40 mL) at 25° C. The resulting mixture was extracted with EA (3×50 mL) and dried over anhydrous Na₂SO₄. After filtration, the filtrate was concentrated under reduced pressure. The residue was purified by silica gel column chromatography, eluted with MeOH:DCM (1:10) to afford tert-butyl (3R)-3-(2-amino-7-chloro-1,3-benzodiazol-1-yl)azepane-1-carboxylate (4.8 g, 82.8%) as a red solid. MS (ESI) calcd for C₁₇H₂₆ClN₃O_{2,364.10}m/z, found 365.10 [M+H]+

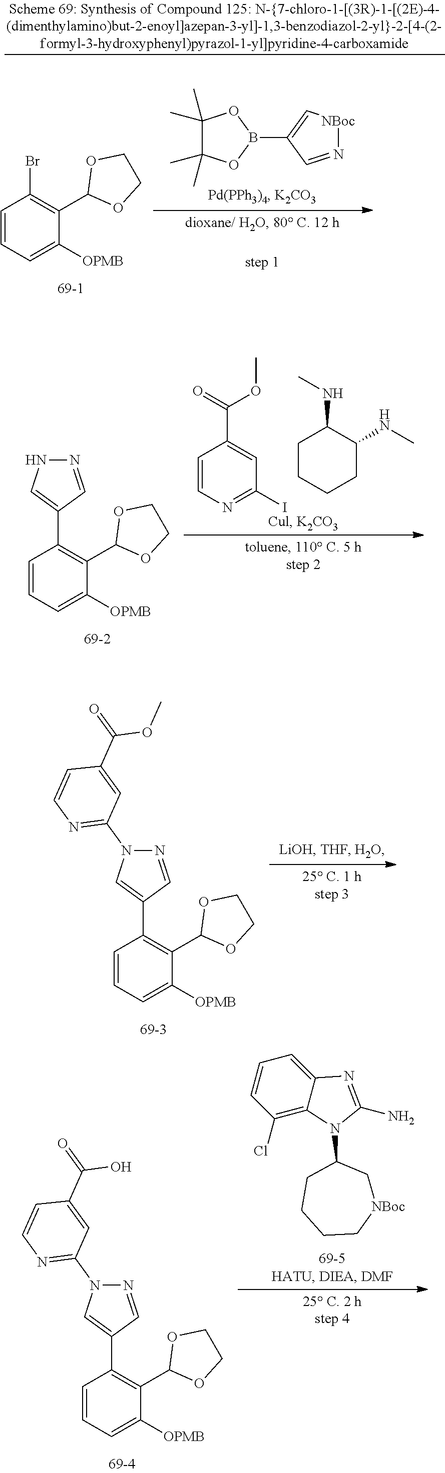

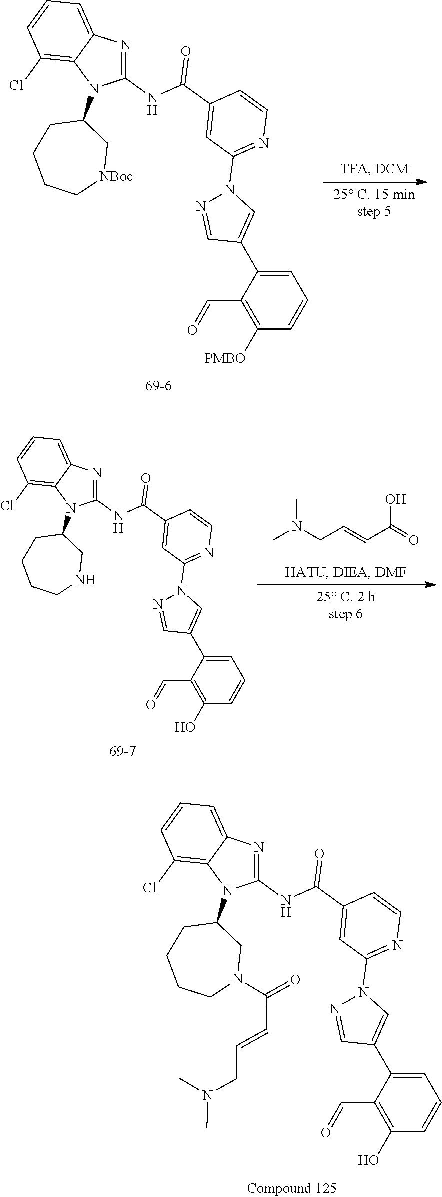

### Step 1: Synthesis of 4-[2-(1,3-Dioxolan-2-yl)-3-[(4-methoxyphenyl)methoxy]phenyl]-1H-pyrazole

To a stirred solution of 2-{2-bromo-6-[(4-methoxyphenyl)methoxy]phenyl}-1,3-dioxolane (500 mg, 1.36 mmol, 1 equiv) in 1,4-dioxane (10 mL) and H₂O (2 mL) was added tert-butyl 4-(4,4,5,5-tetramethyl-1,3,2-dioxaborolan-2-yl)pyrazole-1-carboxylate (523.5 mg, 1.78 mmol, 1.3 equiv) and Pd(PPh₃)₄(155 mg, 0.136 mmol, 0.1 equiv) and K₂CO₃ (281 mg, 2.04 mmol, 1.5 equiv) at 22° C. under nitrogen atmosphere. The resulting mixture was stirred for 12 h at 80° C. under nitrogen atmosphere. Desired product was detected by LCMS. The reaction was quenched with 15 mL H₂O at 22° C. The resulting mixture was extracted with EtOAc (50 mL×3). The combined organic layers were washed with NaCl (aq. 15 mL x 3), dried over anhydrous Na₂SO₄. The resulting mixture was concentrated under vacuum. The residue was purified by silica gel column chromatography, eluted with EA/PE (0-100%) to afford 4-[2-(1,3-dioxolan-2-yl)-3-[(4-methoxyphenyl)methoxy]phenyl]-1H-pyrazole (515 mg, 89.6%) as a yellow solid. MS (ESI) calcd. for C₂₀H₂₀N₂O₄, 352.14 m/z, found 353.05 [M+H]⁺.

### Step 2: Synthesis of 2-(benzyloxy)-3-fluoro-S-(4,4,5,5-tetramethyl-1,3,2-dioxaborolan-2-yl)benzaldehyde

To a stirred solution of 4-[2-(1,3-dioxolan-2-yl)-3-[(4-methoxyphenyl)methoxy]phenyl]-1H-pyrazole (480 mg, 1.36 mmol, 1 equiv) in toluene (10 mL) was added methyl 2-iodopyridine-4-carboxylate (465.7 mg, 1.77 mmol, 1.3 equiv), K₂CO₃ (376.51 mg, 2.724 mmol, 2 equiv), (1S, 2S)—N1,N2-dimethylcyclohexane-1,2-diamine (38.7 mg, 0.27 mmol, 0.2 equiv) and CuI (18 mg, 0.13 mmol, 0.1 equiv) at 22° C. under nitrogen atmosphere. The resulting mixture was stirred for 5 h at 110° C. under nitrogen atmosphere. Desired product was detected by LCMS. The resulting mixture was concentrated under vacuum. The residue was purified by silica gel column chromatography, eluted with EA/PE (0-50%) to afford methyl 2-{4-[2-(1,3-dioxolan-2-yl)-3-[(4-methoxyphenyl)methoxy]phenyl]pyrazol-1-yl}pyridine-4-carboxylate (180 mg, 27.1%) as an off-white solid. MS (ESI) calcd. for C₂₇H₂₅N₃O₆, 487.17 m/z, found 488.05 [M+H]⁺.

### Step 3: Synthesis of 2-{4-[2-(1,3-dioxolan-2-yl)-3-[(4-methoxyphenyl)methoxy]phenyl]pyrazol-1-yl}pyridine-4-carboxylic acid

A mixture of methyl 2-{4-[2-(1,3-dioxolan-2-yl)-3-[(4-methoxyphenyl)methoxy]phenyl]pyrazol-1-yl}pyridine-4-carboxylate (230 mg, 0.47 mmol, 1 equiv) and LiOH (16.9 mg, 0.70 mmol, 1.5 equiv) in THF (5 mL) and H₂O (1 mL) was stirred for 1 h at 25° C. Desired product was detected by LCMS. The resulting mixture was concentrated under reduced pressure. The residue was purified by reversed-phase flash chromatography with the following conditions: column, C18 column; mobile phase, Mobile Phase A: Water (0.1% FA), Mobile Phase B: ACN; Flow rate: 80 m/min 0% to 40% gradient in 40 min, detector, UV 254 nm. This resulted in 2-{4-[2-(1,3-dioxolan-2-yl)-3-[(4-methoxyphenyl)methoxy]phenyl]pyrazol-1-yl}pyridine-4-carboxylic acid (200 mg, 77.6%) as a white solid. MS (ESI) calcd. for C₂₆H₂₃N₃O₆, 473.05 m/z, found 474.05 [M+H]⁺.

### Step 4: Synthesis of tert-butyl (3R)-3-{7-chloro-2-[2-(4-{2-formyl-3-[(4-methoxyphenyl)methoxy]phenyl}pyrazol-1-yl)pyridine-4-amido]-1,3-benzodiazol-1-yl}azepane-1-carboxylate

A solution of 2-{4-[2-(1,3-dioxolan-2-yl)-3-[(4-methoxyphenyl)methoxy]phenyl]pyrazol-1-yl}pyridine-4-carboxylic acid (160 mg, 0.33 mmol, 1 equiv) and tert-butyl (3R)-3-(2-amino-7-chloro-1,3-benzodiazol-1-yl)azepane-1-carboxylate (123.3 mg, 0.338 mmol, 1 equiv) and HATU (154.1 mg, 0.40 mmol, 1.2 equiv) and DIEA (130.8 mg, 1.01 mmol, 3 equiv) in DMF (5 mL) was stirred at 25° C. for 2 h. Desired product was detected by LCMS. Desired product was detected by LCMS. The residue was purified by reversed-phase flash chromatography with the following conditions: column, C18 column; mobile phase, Mobile Phase A: Water(0.1% FA), Mobile Phase B: ACN; Flow rate: 80 mL/min 0% to 100% gradient in 40 min; detector, UV 254 nm. This resulted in tert-butyl (3R)-3-{7-chloro-2-[2-(4-{2-formyl-3-[(4-methoxyphenyl)methoxy]phenyl}pyrazol-1-yl)pyridine-4-amido]-1,3-benzodiazol-1-yl}azepane-1-carboxylate (100 mg, 36.2%) as a yellow solid. MS (ESI) calcd. for C₄₂H₄₂ClN₇O₆, 775.35 m/z, found 776.35 [M+H]⁺.

### Step 5: Synthesis of N-{1-[(3R)-azepan-3-yl]-7-chloro-1,3-benzodiazol-2-yl}-2-[4-(2-formyl-3-hydroxyphenyl)pyrazol-1-yl]pyridine-4-carboxamide

A solution of tert-butyl (3R)-3-{7-chloro-2-[2-(4-{2-formyl-3-[(4-methoxyphenyl)methoxy]phenyl}pyrazol-1-yl)pyridine-4-amido]-1,3-benzodiazol-1-yl}azepane-1-carboxylate (180 mg, 0.23 mmol, 1 equiv) in TFA (0.4 mL) and DCM (2 mL) was stirred for 15 min at 25° C. Desired product was detected by LCMS. The mixture was concentrated under reduced pressure. This resulted in N-{1-[(3R)-azepan-3-yl]-7-chloro-1,3-benzodiazol-2-yl}-2-[4-(2-formyl-3-hydroxyphenyl)pyrazol-1-yl]pyridine-4-carboxamide (120 mg, 88.1%) as a yellow solid. MS (ESI) calcd. for C₄₂H₄₂ClN₇O₆, 555.15m/z, found 556.15 [M+H]⁺.

### Step 6: Synthesis of N-{7-chloro-1-[(3R)-1-[(2E)-4-(dimethylamino)but-2-enoyl]azepan-3-yl]-1,3-benzodiazol-2-yl}-2-[4-(2-formyl-3-hydroxyphenyl)pyrazol-1-yl]pyridine-4-carboxamide

A solution of N-{1-[(3R)-azepan-3-yl]-7-chloro-1,3-benzodiazol-2-yl}-2-[4-(2-formyl-3-hydroxyphenyl)pyrazol-1-yl]pyridine-4-carboxamide (110 mg, 0.19 mmol, 1 equiv) and (2E)-4-(dimethylamino)but-2-enoic acid (25.5 mg, 0.19 mmol, 1 equiv) and HATU (90.2 mg, 0.23 mmol, 1.2 equiv) and DIEA (153.4 mg, 1.18 mmol, 6 equiv) in DMF (2 mL) was stirred at 25° C. for 2 h. Desired product was detected by LCMS. The mixture was purified by Prep-HPLC with the following conditions (Column: XBridge Prep OBD C18 Column, 30*150 mm, 5 μm; Mobile Phase A: Water(10 mmol/L TFA), Mobile Phase B: ACN; Flow rate: 60 m/min; Gradient: 38% B to 68% B in 7 min, 68% B; Wave Length: 254 nm; RT1(min): 5.98; Number Of Runs: 0) to afford N-{7-chloro-1-[(3R)-1-[(2E)-4-(dimethylamino)but-2-enoyl]azepan-3-yl]-1,3-benzodiazol-2-yl}-2-[4-(2-formyl-3-hydroxyphenyl)pyrazol-1-yl]pyridine-4-carboxamide (16.3 mg, 12.3%) as a yellow solid. MS (ESI) calcd. for C₃₅H₃₅ClN₈04, 666.30 m/z, found 667.30 [M+H]⁺. ¹H NMR (400 MHz, DMSO-d₆) δ 13.33-13.35 (m, 1H), 11.79 (d, J=4.9 Hz, 1H), 10.20 (d, J=2.6 Hz, 1H), 9.64 (s, 1H), 9.01 (d, J=4.0 Hz, 1H), 8.81 (s, 1H), 8.70 (dd, J=5.1, 3.0 Hz, 1H), 7.99 (ddd, J=6.7, 5.0, 1.4 Hz, 1H), 7.64 (ddt, J=7.8, 5.4, 1.4 Hz, 2H), 7.39-7.25 (m, 2H), 7.13 (ddd, J=7.5, 2.6, 1.1 Hz, 1H), 7.05-6.98 (m, 2H), 6.82-6.70 (m, 1H), 5.60-5.40 (m, 1H), 4.82-4.43 (m, 1H), 4.40-4.18 (m, 2H), 4.03-3.46 (m, 4H), 2.79 (s, 3H), 2.71 (s, 3H), 2.22 (s, 4H).

The following compounds were synthesized according to the preceding method or analogous methods thereto:

| Cmpd No. | Characterization Data |
|---|---|
| 126 | MS (ESI) calcd. for C₃₂H₃₃ClN₆O₅, 616.25 m/z, found 617.25 [M + H]⁺. ¹H |
| | NMR (400 MHz, DMSO-d₆) δ 13.28 (s, 1H), 10.26 (d, J = 2.3 Hz, 1H), 8.36- |
| | 8.24 (m, 1H), 8.15 (s, 1H), 7.79 (dd, J = 8.2, 5.2 Hz, 1H), 7.70 (s, 2H), 7.67 (s, |
| | 2H), 7.65-7.55 (m, 1H), 6.88 (d, J = 8.4 Hz, 3H), 5.49 (d, J = 9.2 Hz, 1H), |
| | 4.75-4.60 (m, 1H), 4.60 (s, 1H), 4.22 (s, 1H), 3.96 (s, 2H), 3.05 (d, J = 4.3 Hz, |
| | 1H), 2.67-2.57 (m, 1H), 2.32 (s, 3H), 2.22 (s, 3H), 2.16 (s, 4H), 1.99 (s, 1H), |
| | 1.24 (s, 1H). |

### Example 5. Intact Mass Spectrometry and Competition Studies

Identification of covalent binding by intact mass spectrometry for Table 5 compounds For intact mass spectrometry experiments EGFR L⁸⁵⁸R/T790M/C₇₉₇S triple-mutant protein (residues 668-1210, Biortus, 0.8 uM) was incubated with compound from Table 5 (1.5 uM).

For competition experiments investigating specificity of covalent complex formation, the EGFR ATP binding site was saturated with non-covalent ATP-competitive inhibitor (brigatinib, 20 uM) for 30 min at room temperature in buffer (50 mM HEPES 7.4, 10 mM MgCl2, 1% glycerol, 1.2 mM DTT). Freshly dissolved NaBH4 in PBS was added to 5 mM final concentration for 30 min on ice to reduce imines into mass-spec stable amines. Reduction reactions were quenched by addition of 100% volume acetonitrile+1% TFA prior to injection on a Waters BioAccord LC-MS. Table 5 provides compounds 1-8 which were used in these studies.

TABLE 5
Structures of Compounds 1-8 and Reference Compound 1

| No. | Structure |
|---|---|
| 1 | 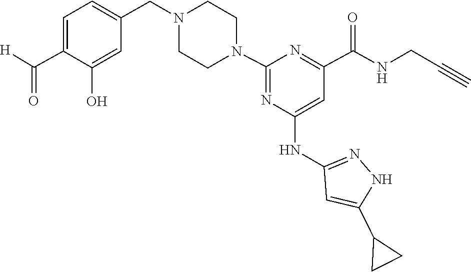 [SMILES: C#CCNC(=O)c1cc(Nc2cc(C3CC3)[nH]n2)nc(N2CCN(Cc3ccc(C=O)c(O)c3)CC2)n1] |
| 2 | 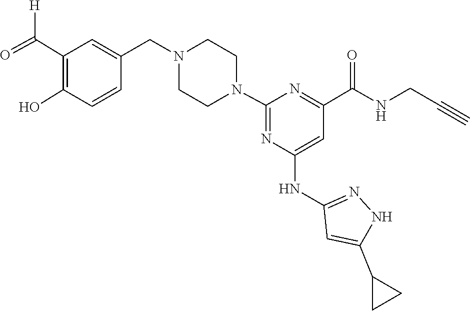 [SMILES: C#CCNC(=O)c1cc(Nc2cc(C3CC3)[nH]n2)nc(N2CCN(Cc3ccc(O)c(C=O)c3)CC2)n1] |
| 3 | 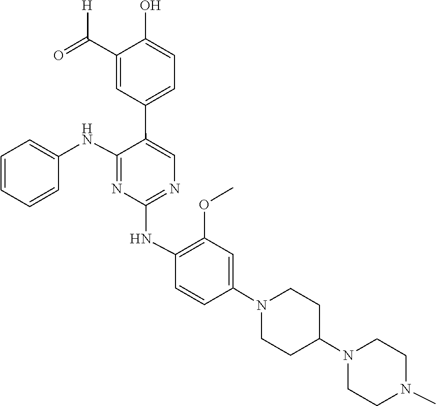 [SMILES: COc1cc(N2CCC(N3CCN(C)CC3)CC2)ccc1Nc1ncc(-c2ccc(O)c(C=O)c2)c(Nc2ccccc2)n1] |
| 4 | 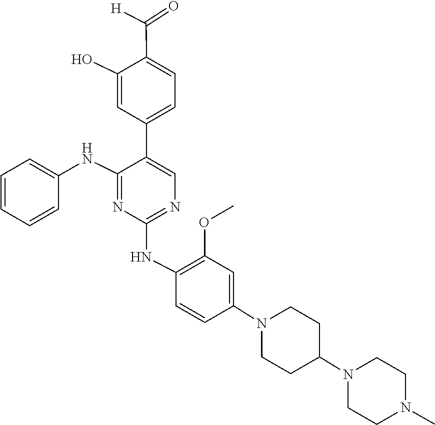 [SMILES: COc1cc(N2CCC(N3CCN(C)CC3)CC2)ccc1Nc1ncc(-c2ccc(C=O)c(O)c2)c(Nc2ccccc2)n1] |
| 5 | 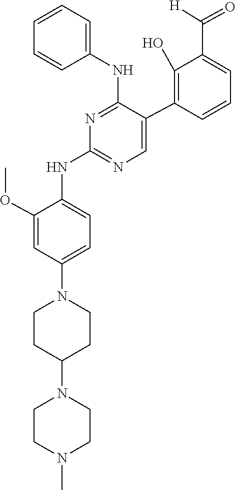 [SMILES: COc1cc(N2CCC(N3CCN(C)CC3)CC2)ccc1Nc1ncc(-c2cccc(C=O)c2O)c(Nc2ccccc2)n1] |
| 6 | 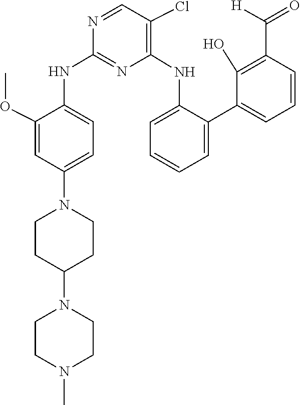 [SMILES: COc1cc(N2CCC(N3CCN(C)CC3)CC2)ccc1Nc1ncc(Cl)c(Nc2ccccc2-c2cccc(C=O)c2O)n1] |
| 7 | 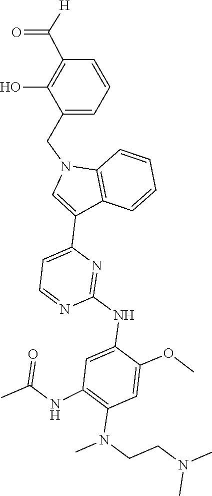 [SMILES: COc1cc(N(C)CCN(C)C)c(NC(C)=O)cc1Nc1nccc(-c2cn(Cc3cccc(C=O)c3O)c3ccccc23)n1] |
| 8 | 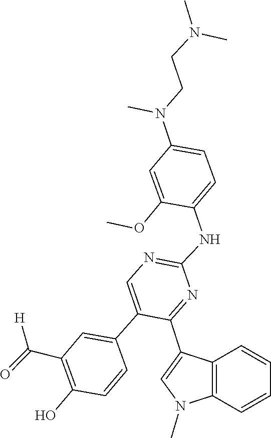 [SMILES: COc1cc(N(C)CCN(C)C)ccc1Nc1ncc(-c2ccc(O)c(C=O)c2)c(-c2cn(C)c3ccccc23)n1] |
| Ref.1 | 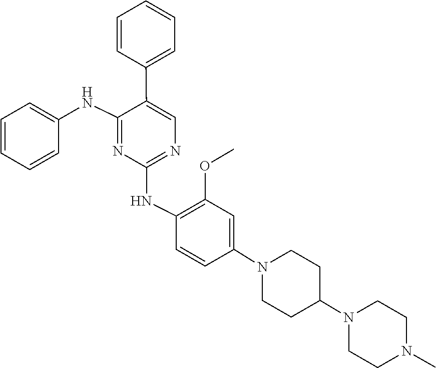 [SMILES: COc1cc(N2CCC(N3CCN(C)CC3)CC2)ccc1Nc1ncc(-c2ccccc2)c(Nc2ccccc2)n1] |

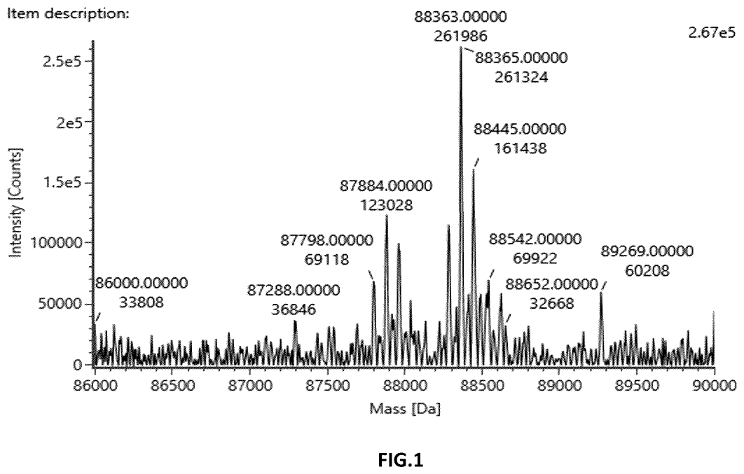

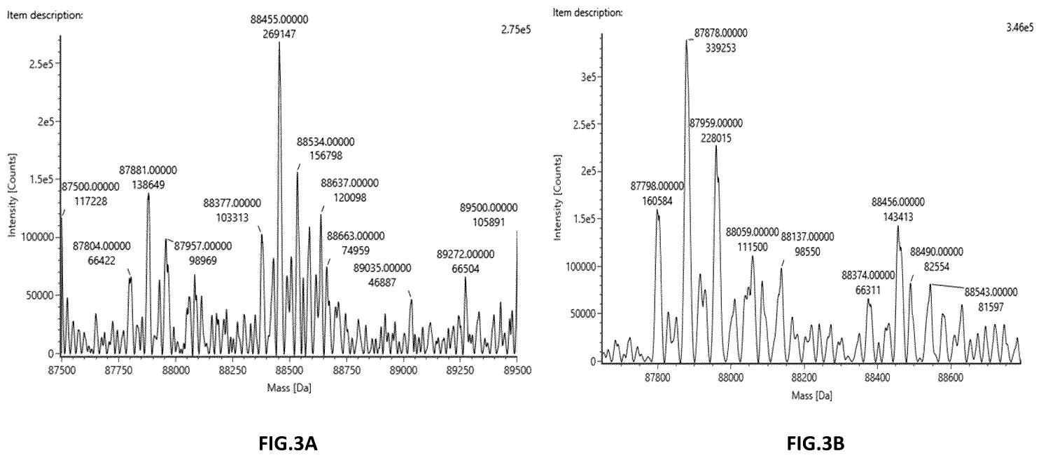

FIGS. 1 to 9 provide intact mass spectra for EGFR L858R/T790M/C797S triple-mutant protein after incubation with compounds of the present disclosure and subsequent borohydride reduction. Sodium borohydride was added to reduce the reversible imine bond between the EGFR protein and compounds of the present disclosure, thereby forming a more stable C—N single bond between the protein and the compound and allowing detection of the covalent complex by intact mass spec. Further, FIGS. 3 to 9 each provide intact mass spectra following the incubation of an EGFR protein with a compound of the present disclosure both (A) in the absence of a competitor molecule (i.e., brigatinib, a molecule known to bind to and inhibit the function of EGFR); and (B) in the presence of a significant stoichiometric excess of a competitor molecule.

As shown in FIG. 1, incubation of Compound 1 with EGFR resulted in a significant level of covalent complex formation between Compound 1 and the EGFR protein. The data provided in FIG. 1 demonstrates two different sets of m/z peaks, which correspond to the mass of the unmodified EGFR protein and the covalent complex between Compound 1 and EGFR.

The first set of peaks on left side of the chart (between mass values of 87700 and 88100 Da, and including specific observed mass values of 87798 and 87884 Da) corresponds to the unmodified EGFR protein, with the different individual peaks corresponding to different phosphorylation states of the protein. The second set of peaks to the right of the first set (between mass values of 88200 and 88700 Da, and including specific observed mass values of 88363 and 88445 Da) corresponds to the covalent complex formed between Compound 1 and EGFR protein, with the different individual peaks corresponding to different phosphorylation states of the protein-compound complex. Thus, as seen in FIG. 1 a significant amount of covalent complex was formed between Compound 1 and the EGFR protein, indicated by relative intensity of the peaks corresponding to the covalent construct compared to those corresponding to the unmodified protein. Furthermore, the extent of covalent complex formation was likely to be even higher than shown in FIG. 1, as some of the peak intensity for the peaks corresponding to unmodified protein was attributable to fragmentation of the C—N bond between Compound 1 and the EGFR protein (formed by borohydride reduction of the imine bond in the covalent complex) during ionization and detection.

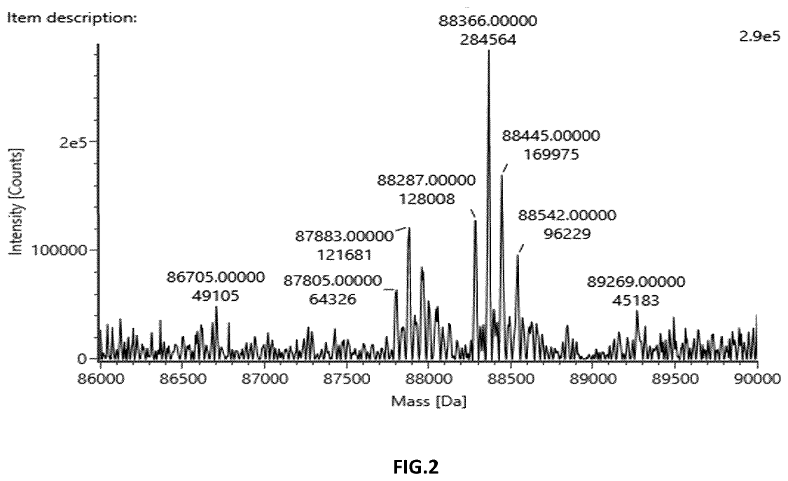

As shown in FIG. 2, incubation of Compound 2 with EGFR resulted in a significant level of covalent complex formation between Compound 2 and the EGFR protein. The first set of peaks on left side of the chart (between mass values of 87700 and 88100 Da, and including specific observed mass values of 87805 and 87883 Da) corresponds to the unmodified EGFR protein. The second set of peaks to the right of the first set (between mass values of 88200 and 88700 Da, and including specific observed mass values of 88287, 88366, 88445, and 88542 Da) corresponds to the covalent complex formed between Compound 2 and EGFR protein.

FIG. 3 provides intact mass spectra following incubation of Compound 3 with EGFR both in the absence of any competitor molecule brigatinib (FIG. 3A) and in the presence of a significant molar excess of competitor molecule brigatinib (FIG. 3B). As seen in FIG. 3A, a significant amount of covalent complex was observed (set of peaks between mass values of 88300 and 88700 Da, and including specific observed mass values of 88377, 88455, and 88534 Da), with a small amount of unmodified EGFR protein (or fragmentation of the covalent complex), indicated by the set of peaks between mass values of 87700 and 88100 Da, including specific observed mass values of 87804, 87881, and 87957 Da. In contrast, FIG. 3B shows essentially no covalent complex formation when EGFR was incubated with Compound 3 in the presence of a significant stoichiometric excess of a competitor molecule brigatinib, and instead only mass peaks corresponding to the unmodified protein (set of peaks between mass values of 87700 and 88100 Da, and including specific observed mass values of 87798, 87878, and 87959 Da. Thus, the data provided in FIGS. 3A and 3B show that (1) Compound 3 formed a covalent complex with EGFR, and (2) Compound 3 bound to the same binding pocket of EGFR as the competitor molecule, as shown by the significant reduction in observed complex formation in the presence of a stoichiometric excess of competitor molecule (compare FIG. 3B to FIG. 3A).

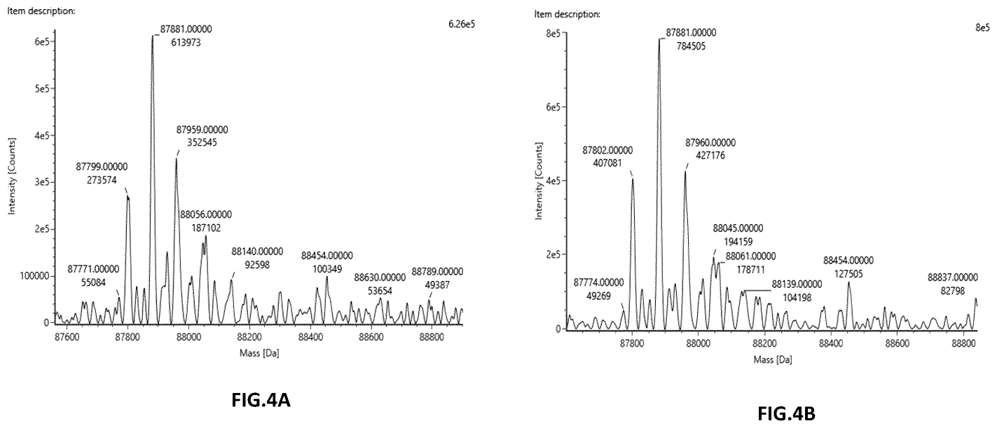

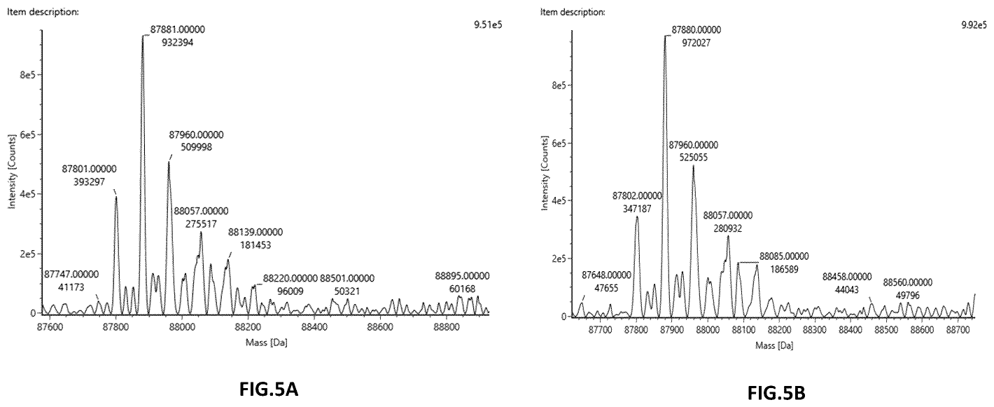

FIGS. 4 and 5 provide intact mass spectra following incubation of Compound 4 and Compound 5, respectively, with EGFR, both in the absence of any competitor molecule brigatinib (FIG. 4A and 5A) and in the presence of a significant molar excess of competitor molecule (FIG. 4B and 5B). Under both conditions, only peaks corresponding to unmodified protein were observed for both Compound 4 and Compound 5, indicating that either no covalent complex was formed, or the resulting C—N bond completely fragmented during ionization and/or detection.

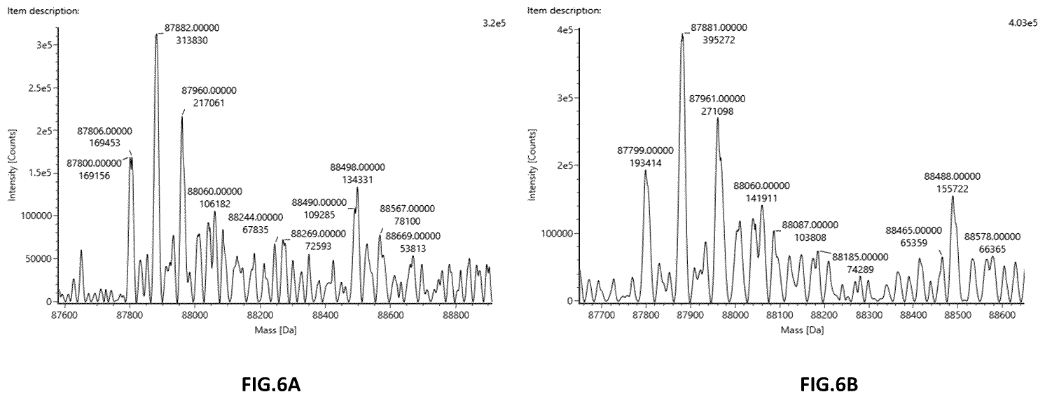

FIG. 6 provide intact mass spectra following incubation of Compound 6 with EGFR, both in the absence of any competitor molecule (FIG. 6A) and in the presence of a significant molar excess of competitor molecule brigatinib (FIG. 6B). FIG. 6A shows the presence of covalent complex (peaks corresponding to masses of 88498 and 88567), as well as significant amount of unmodified protein (peaks corresponding to masses of 87800, 87882, and 87960).

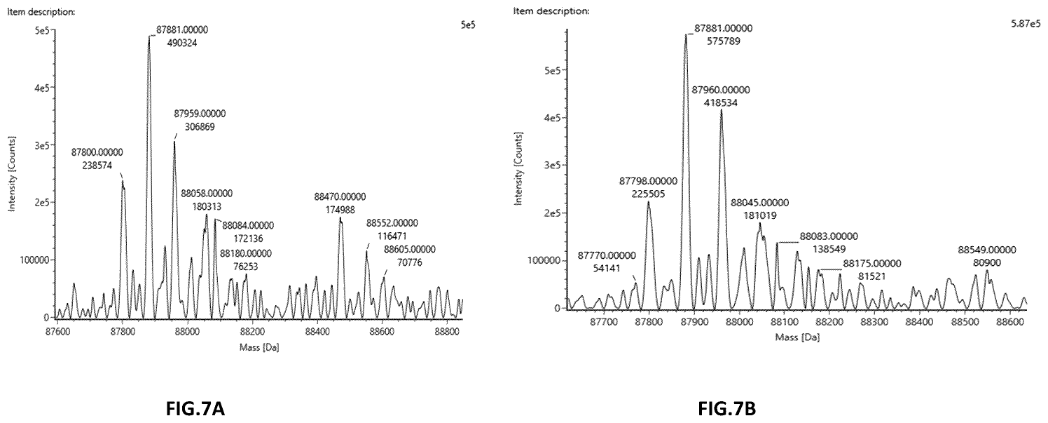

FIG. 7 provides intact mass spectra following incubation of Compound 7 with EGFR, both in the absence of any competitor molecule brigatinib (FIG. 7A) and in the presence of a significant molar excess of competitor molecule (FIG. 7B). FIG. 7A shows the presence of covalent complex (peaks corresponding to masses of 88470 and 88552), as well as significant amount of unmodified protein (peaks corresponding to masses of 87800, 87881, and 87959).

No covalent complex formation is observed in FIG. 7B, indicating that Compound 7 bound to the same EGFR binding pocket as the competitor molecule.

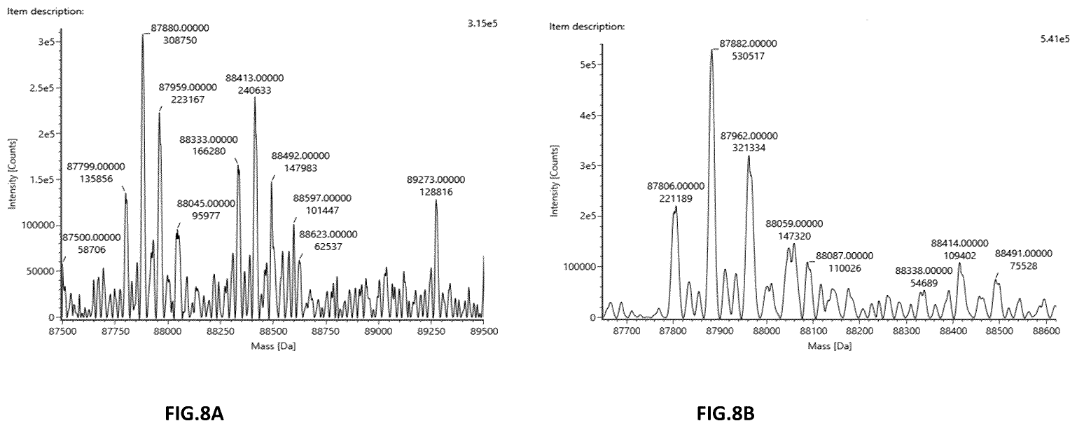

FIG. 8 provides intact mass spectra following incubation of Compound 8 with EGFR, both in the absence of any competitor molecule brigatinib (FIG. 8A) and in the presence of a significant molar excess of competitor molecule (FIG. 8B). FIG. 8A shows the presence of covalent complex (peaks corresponding to masses of 88333, 88413, and 88982), and some unmodified protein (peaks corresponding to masses of 87799, 87880, and 87959). No covalent complex formation was observed in FIG. 8B, indicating that Compound 8 bound to the same EGFR binding pocket as the competitor molecule.

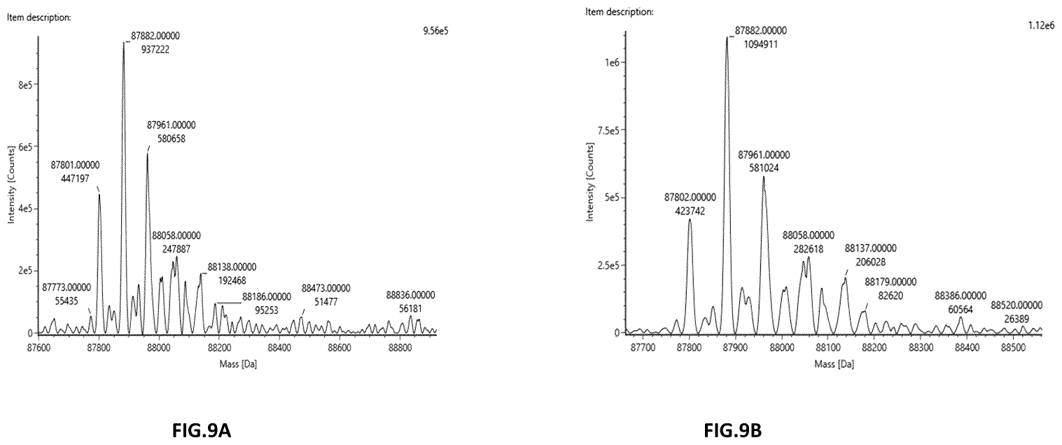

FIG. 9 provide intact mass spectra following incubation of a reference compound (Ref. 1) with EGFR, both in the absence of any competitor molecule brigatinib (FIG. 9A) and in the presence of a significant molar excess of competitor molecule brigatinib (FIG. 9B). Under both conditions, only peaks corresponding to unmodified protein are observed, indicating that either no covalent complex was formed between the reference compound (Ref. 1) and EGFR. This is to be expected, given the absence of a reversible covalent electrophile on the reference compound (Ref. 1).

### Identification of Covalent Binding by Intact Mass Spectrometry for Additional Compounds

EGFR L858R/T790M/C₇₉₇S protein (residues 668-1210, Biortus, 2.2 μM) was incubated with compound (4.4 μM)+/−saturating reversible competitor (BI-4020, 30 uM) for 30 min at RT in buffer (50 mM HEPES 7.4, 10 mM MgCl2, 1% glycerol, 1.2 mM DTT).

Freshly dissolved NaBH₄ in PBS was added to 5 mM final concentration for 5 min at room temperature to reduce imines to mass-spec stable amines. Reduction reactions were quenched by addition of 100% volume acetonitrile+1% TFA prior to injection on a Waters BioAccord L^{C}-MS.

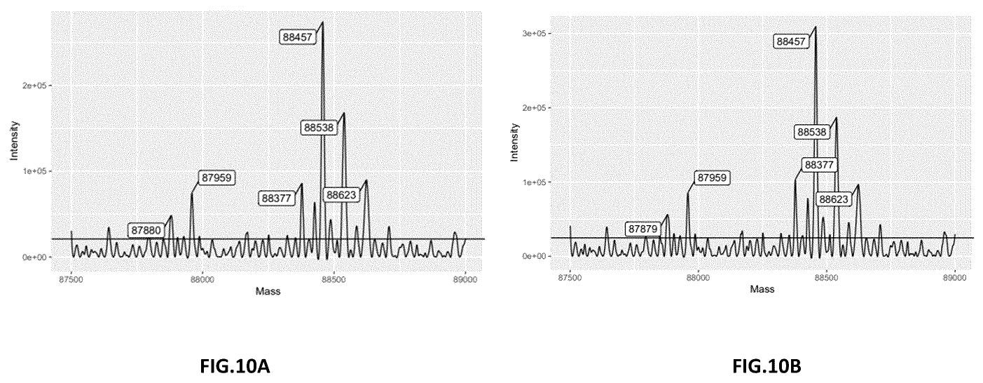

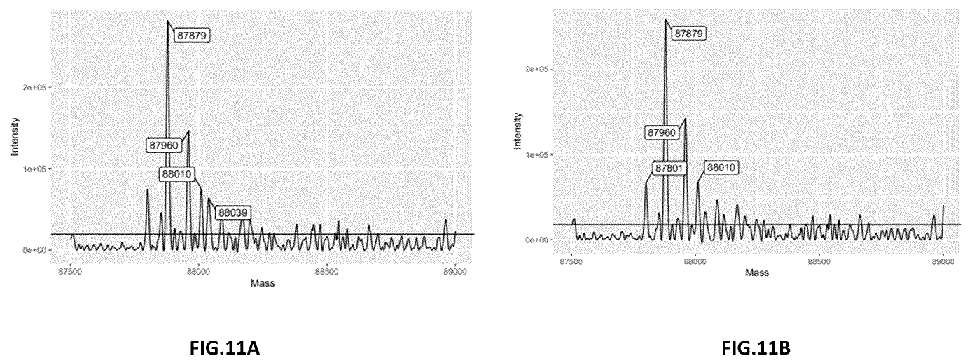

FIGS. 10-11 provide intact mass spectra following incubation of Compound 3, both in the absence of any competitor molecule (FIG. 10) and in the presence of a significant molar excess of competitor molecule BI-4020 (FIG. 11). Each experiment was run in duplicate and the figures show data from each run. The increased intact mass observed in FIG. 10 relative to FIG. 11 corresponds to the covalent complex formed between the protein and Compound 3.

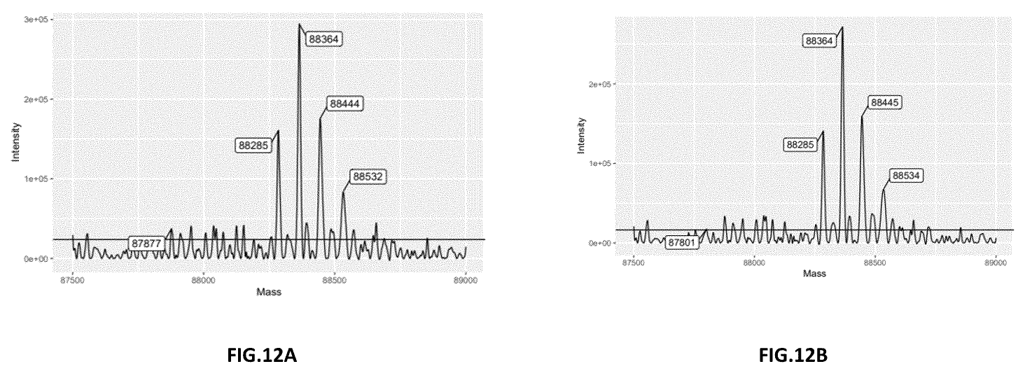

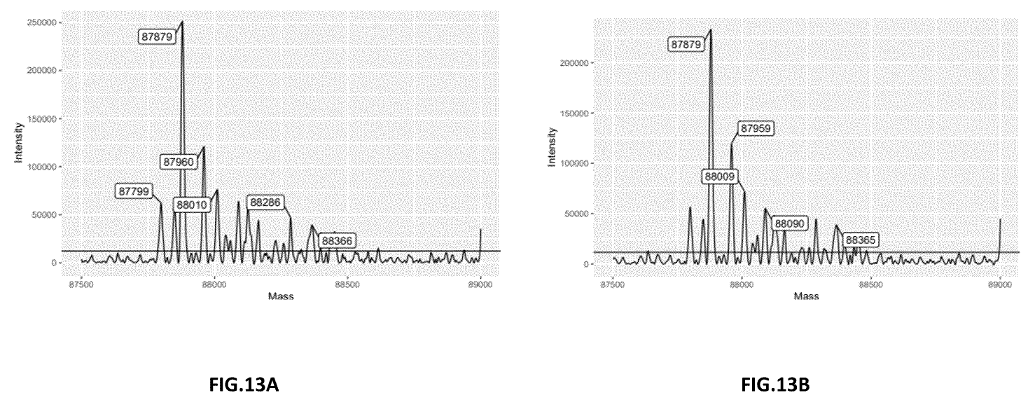

FIGS. 12-13 provide intact mass spectra following incubation of a compound of the present disclosure, both in the absence of any competitor molecule BI-4020 (FIG. 12) and in the presence of a significant molar excess of competitor molecule BI-4020 (FIG. 13).

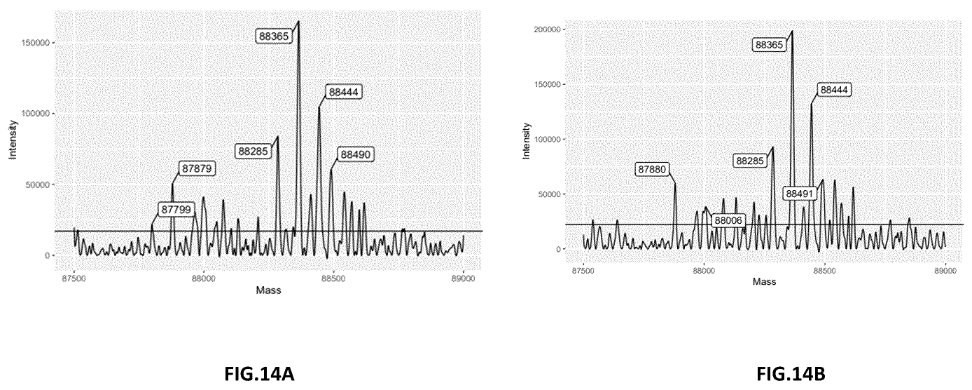

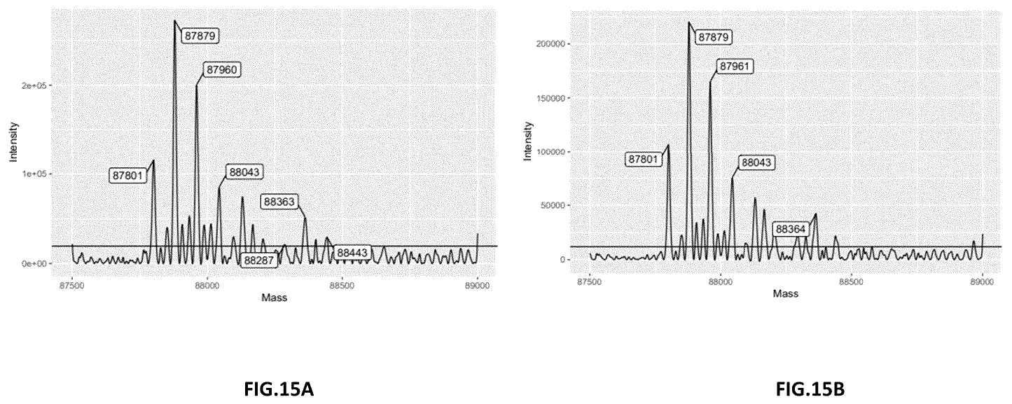

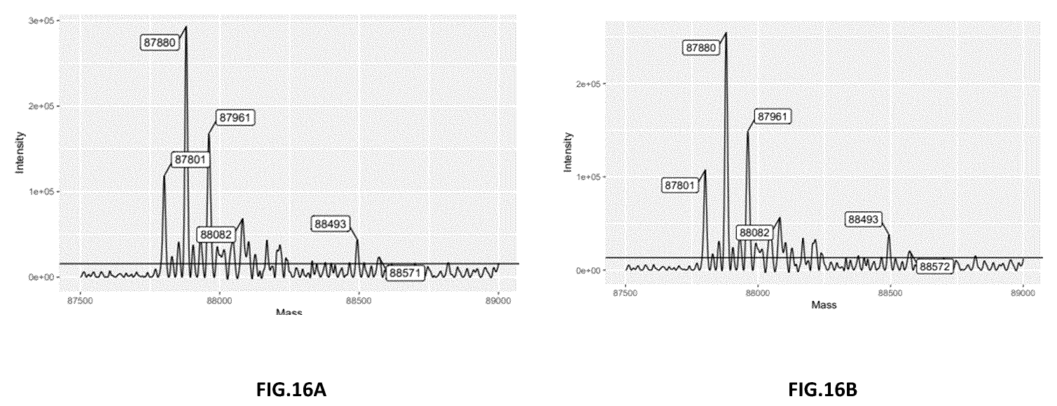

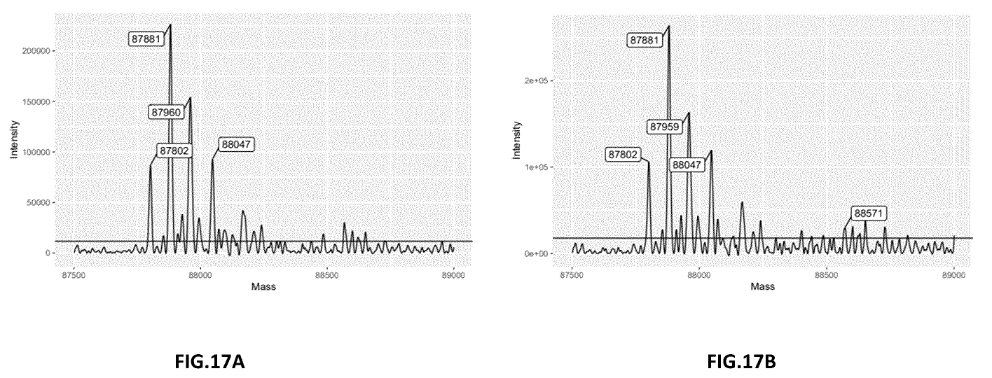

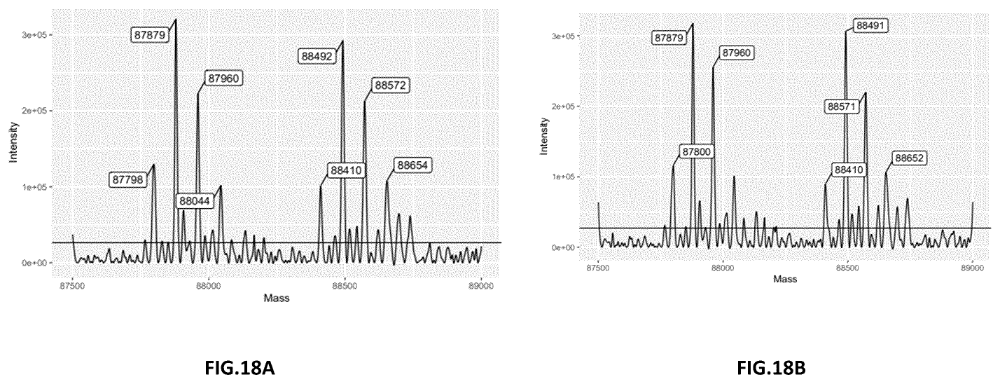

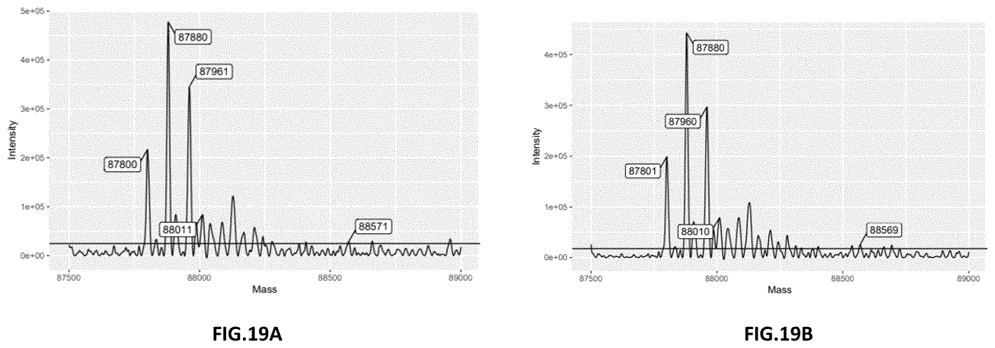

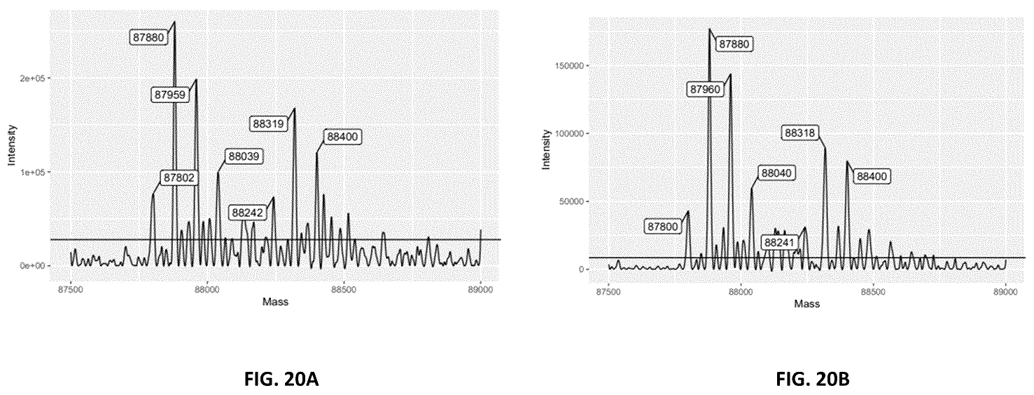

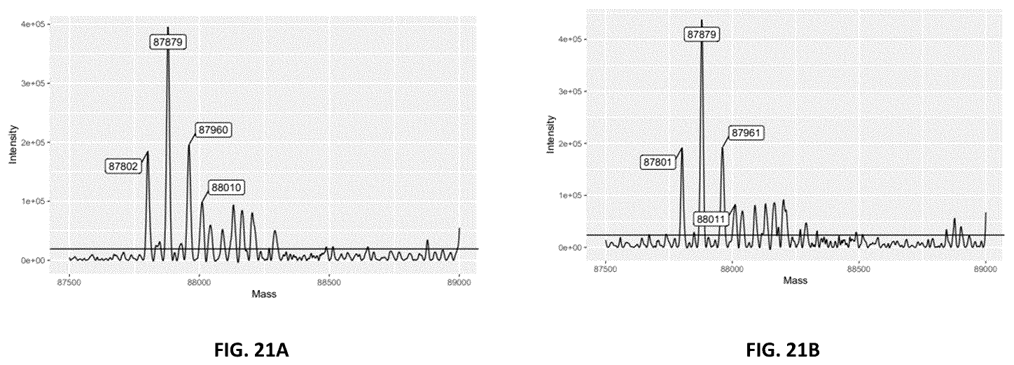

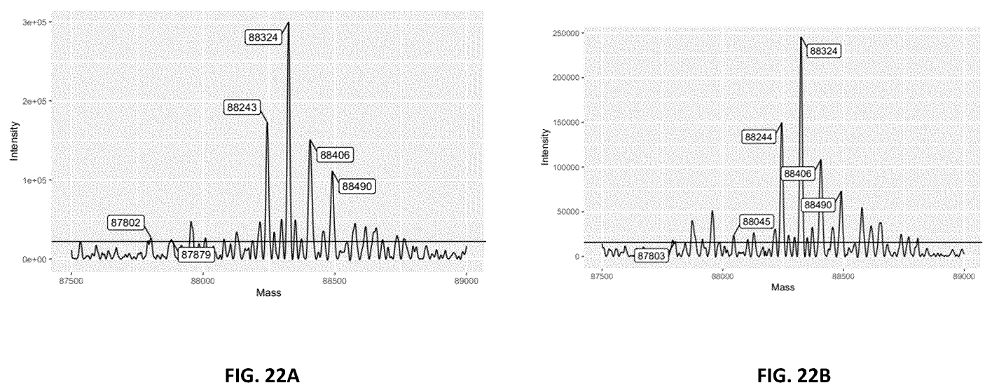

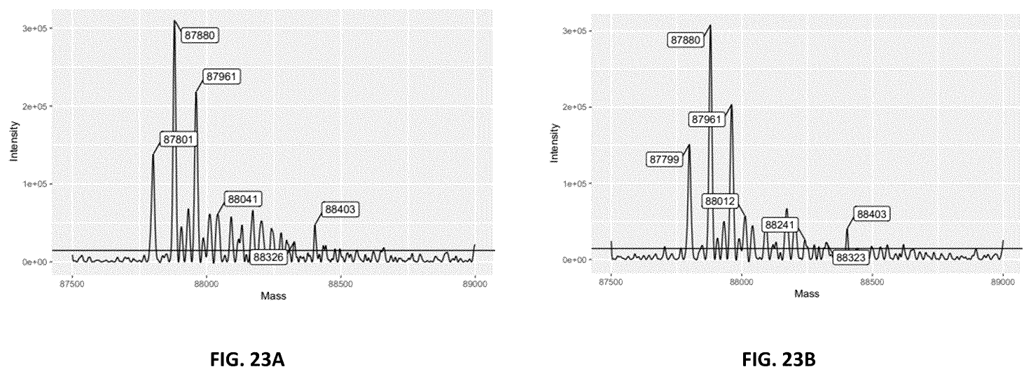

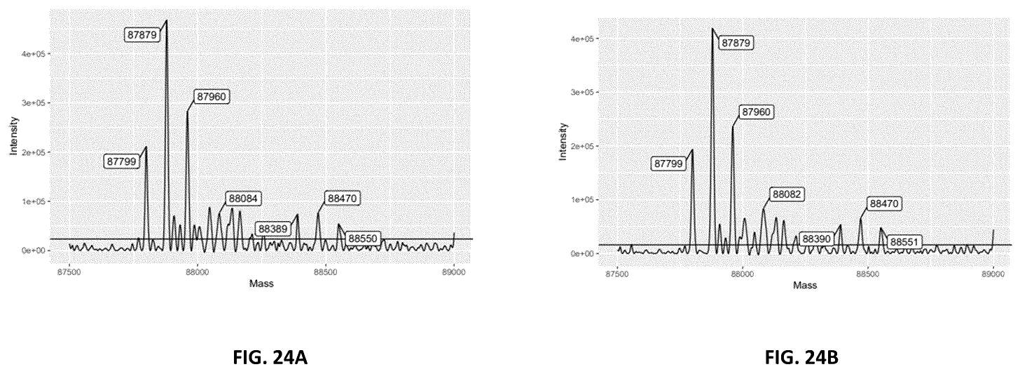

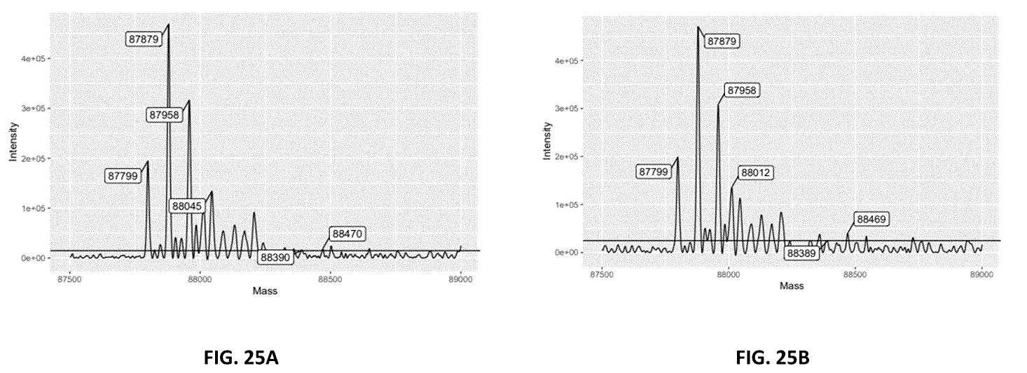

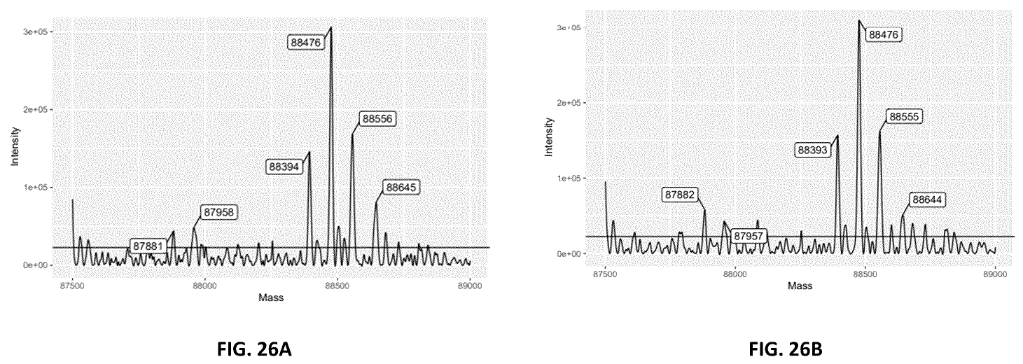

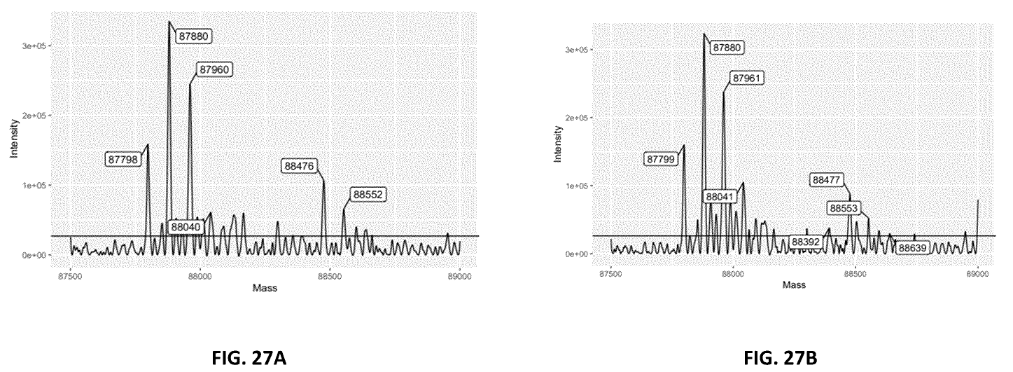

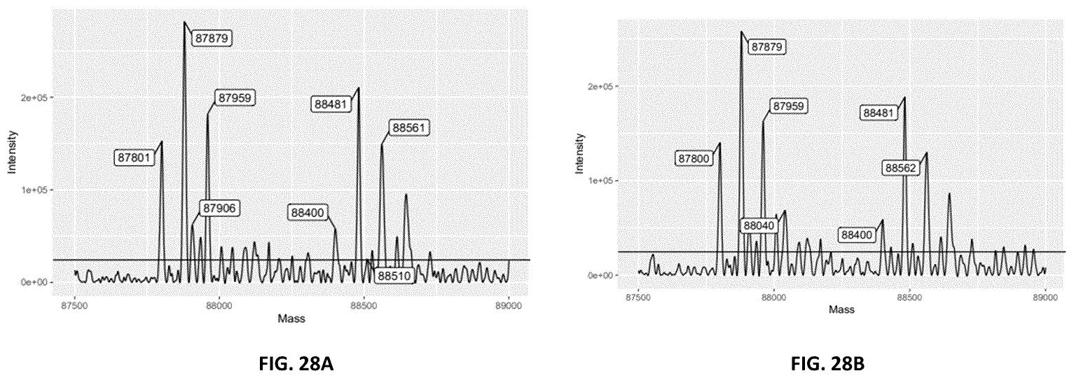

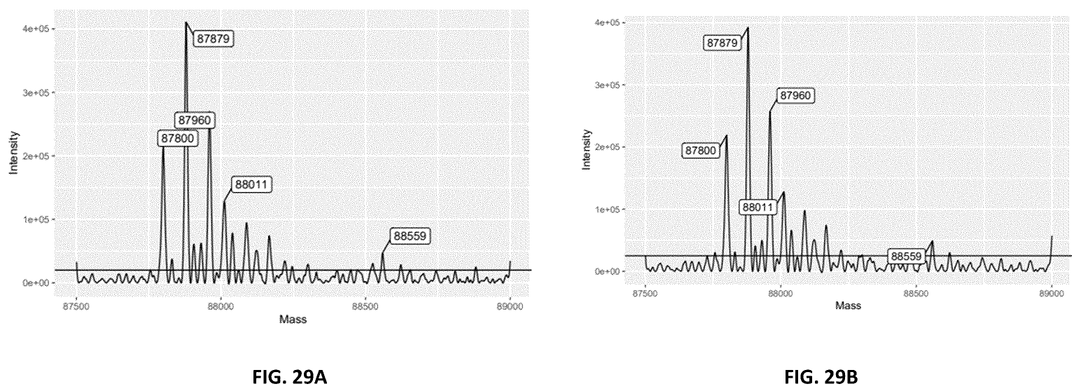

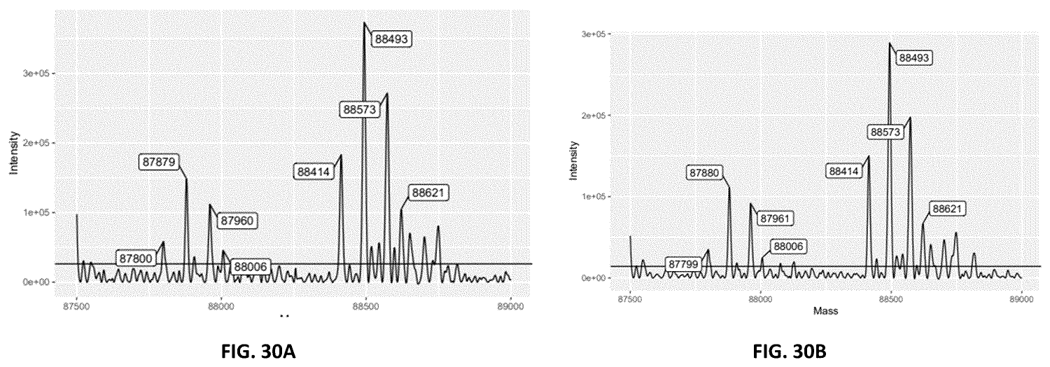

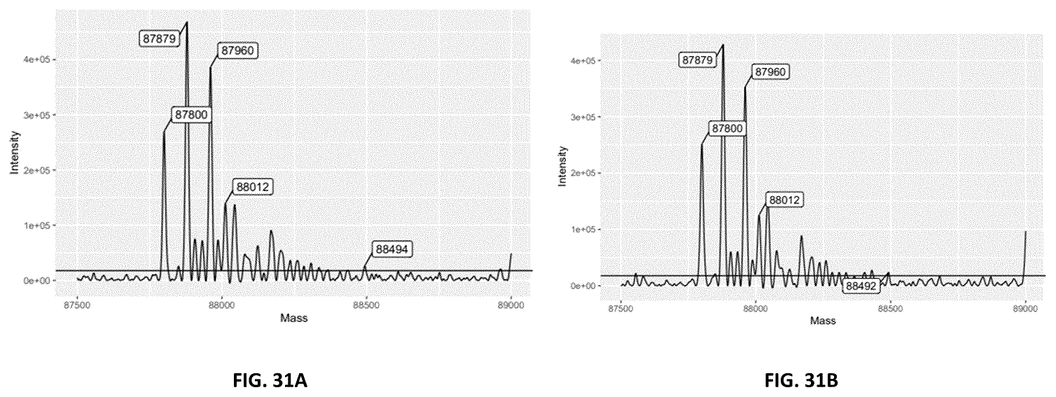

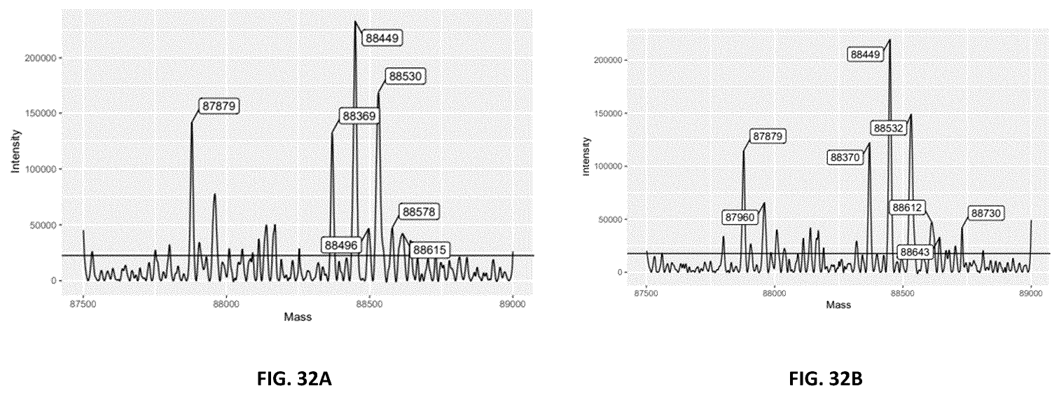

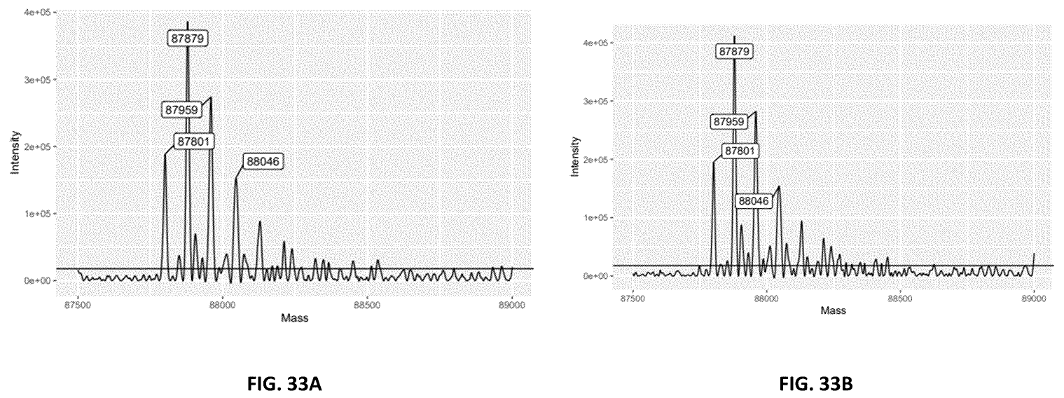

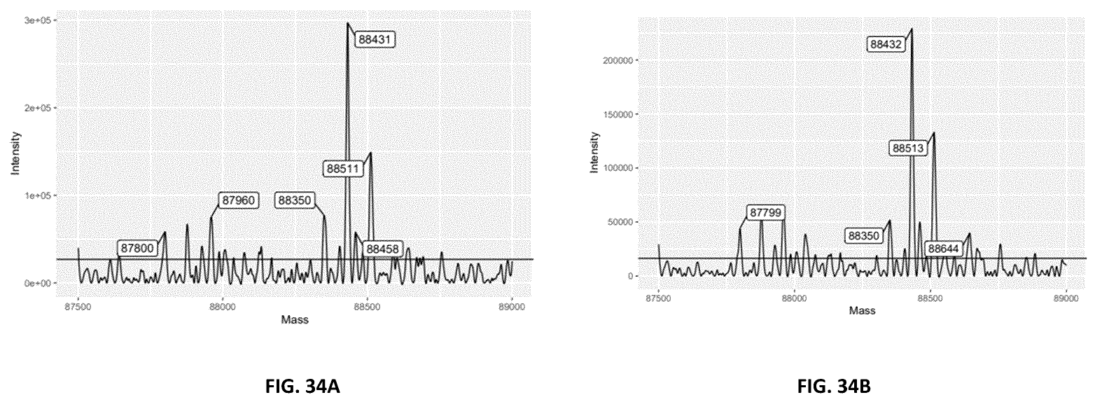

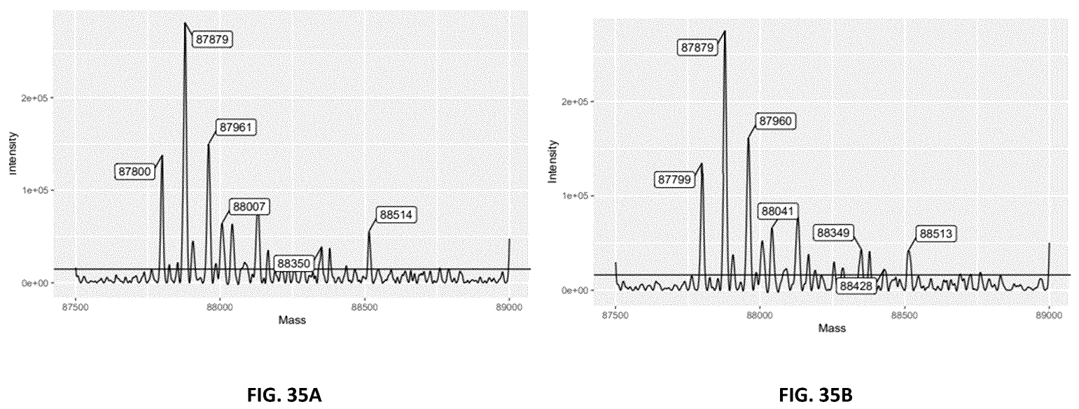

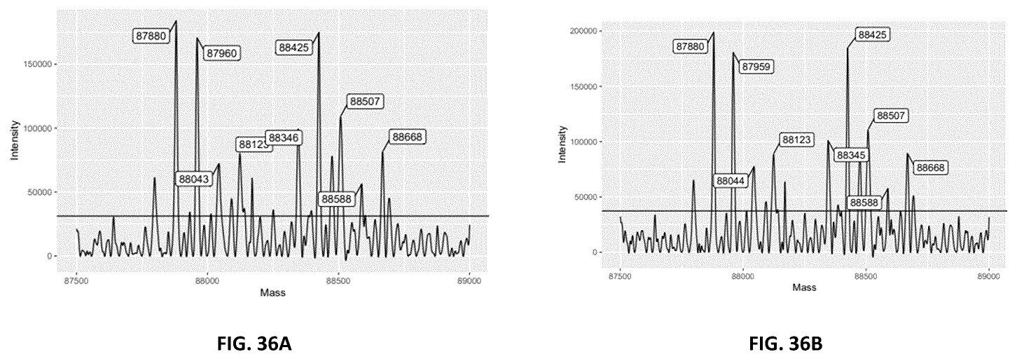

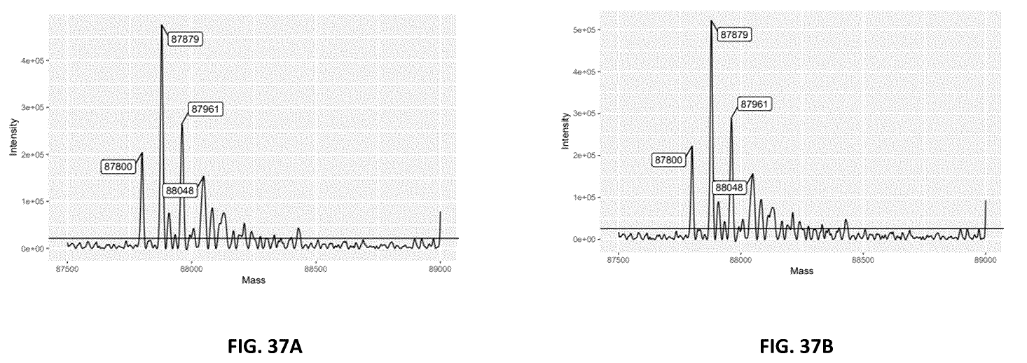

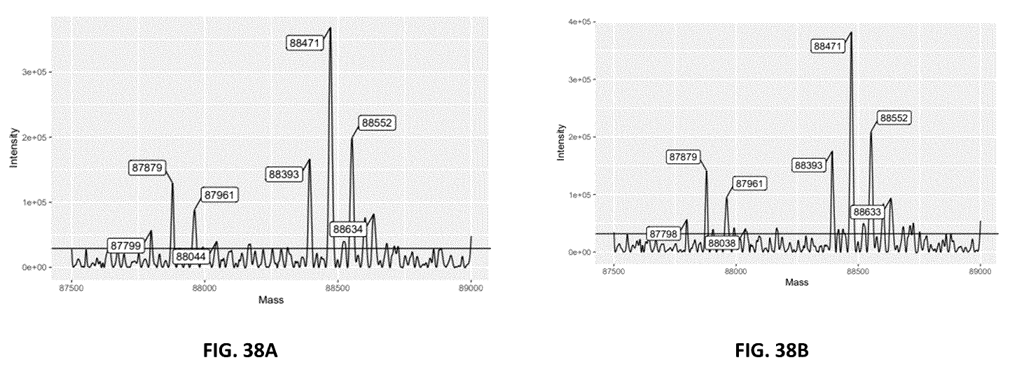

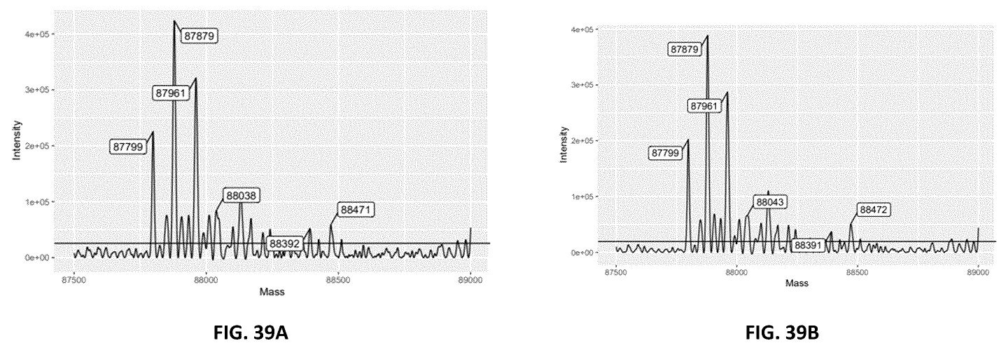

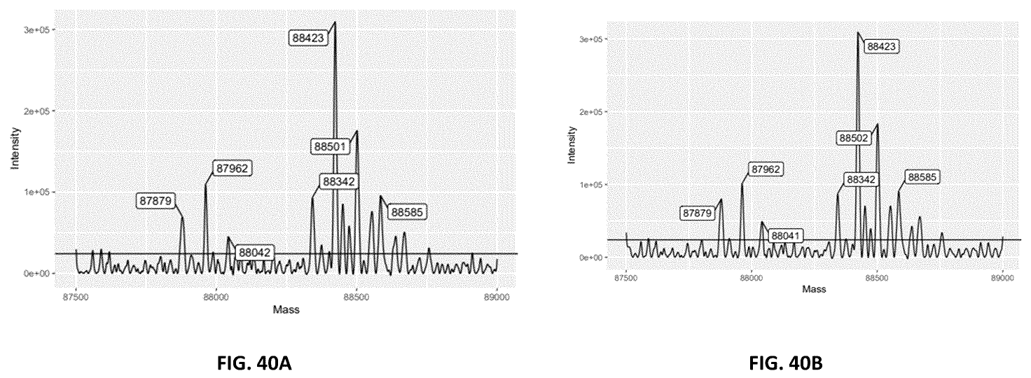

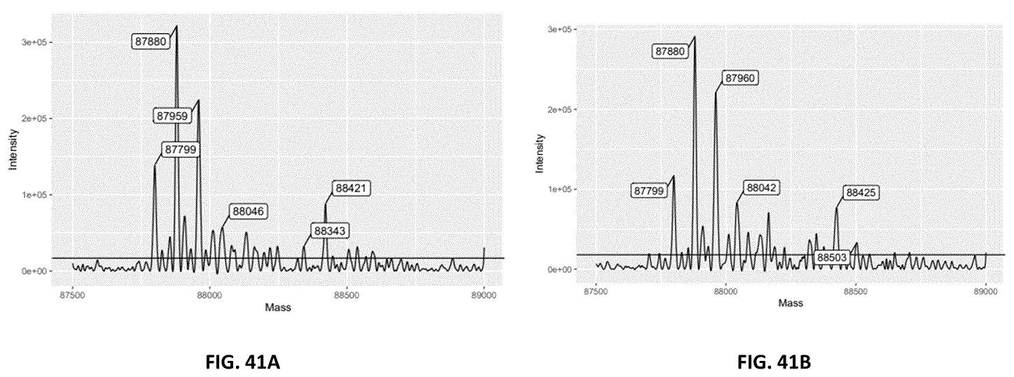

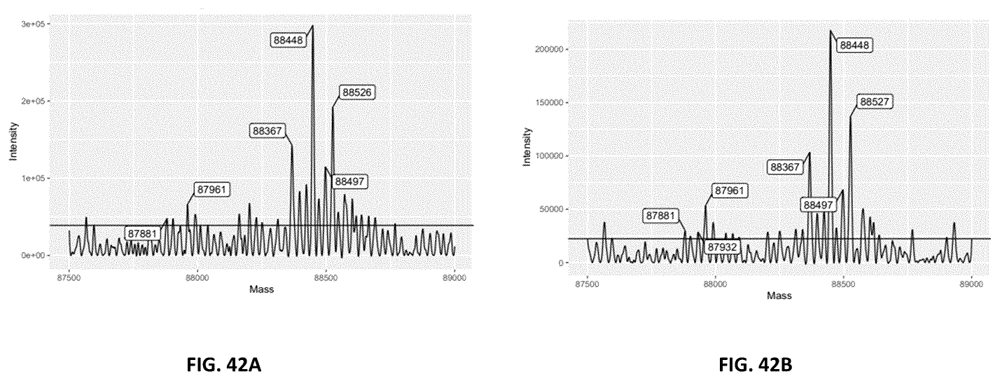

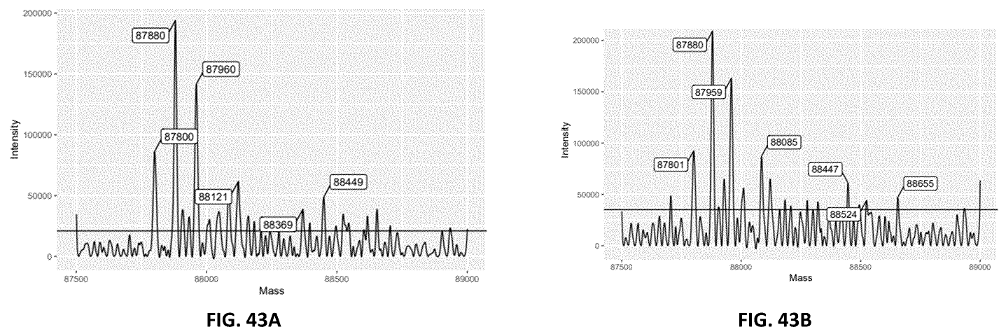

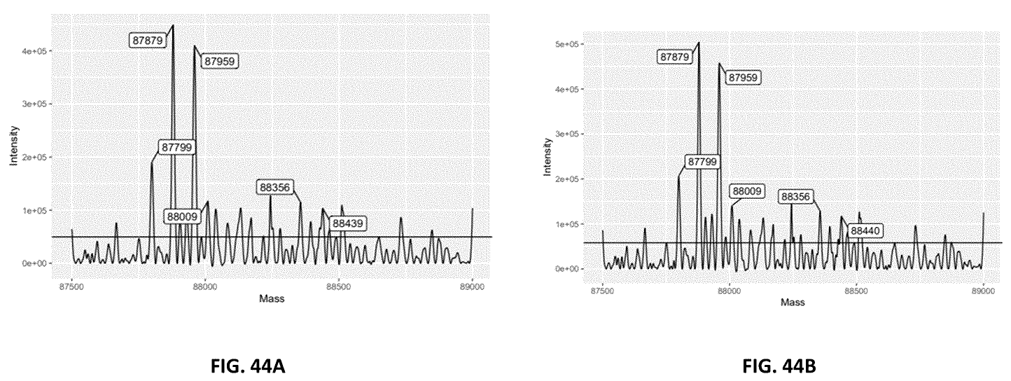

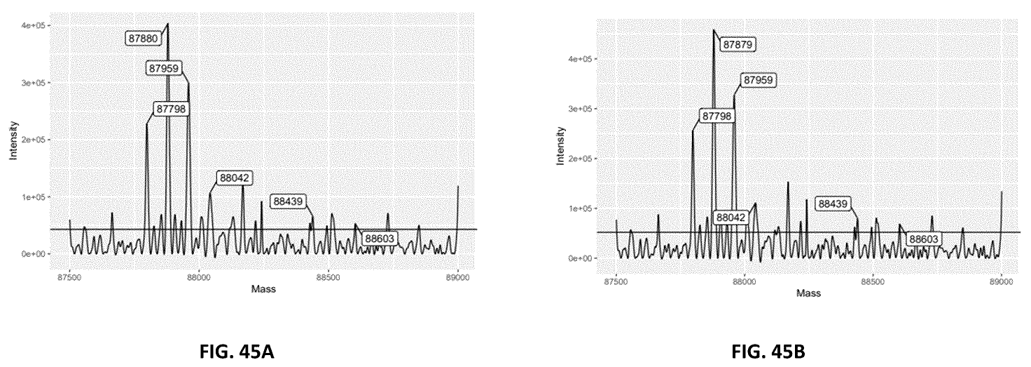

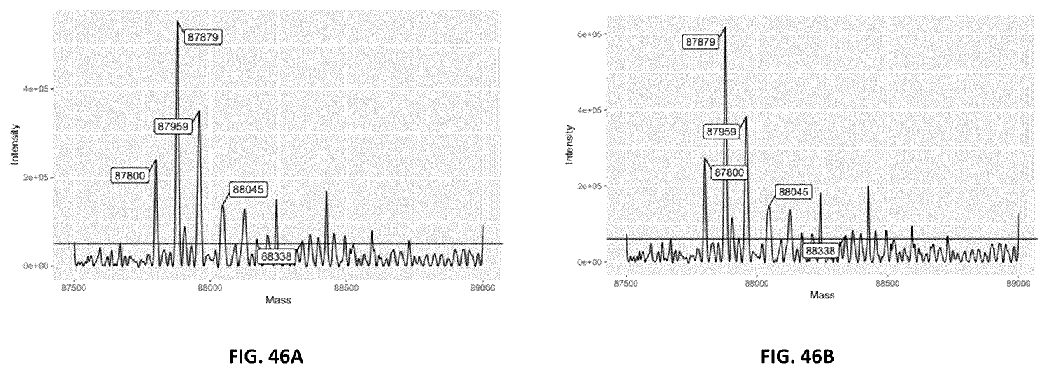

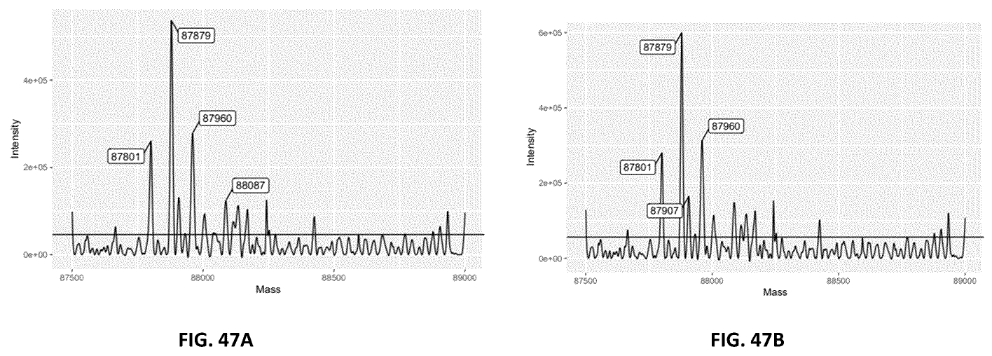

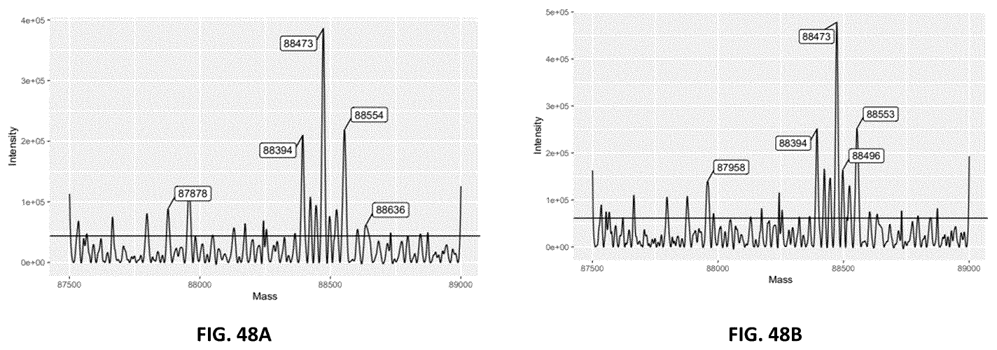

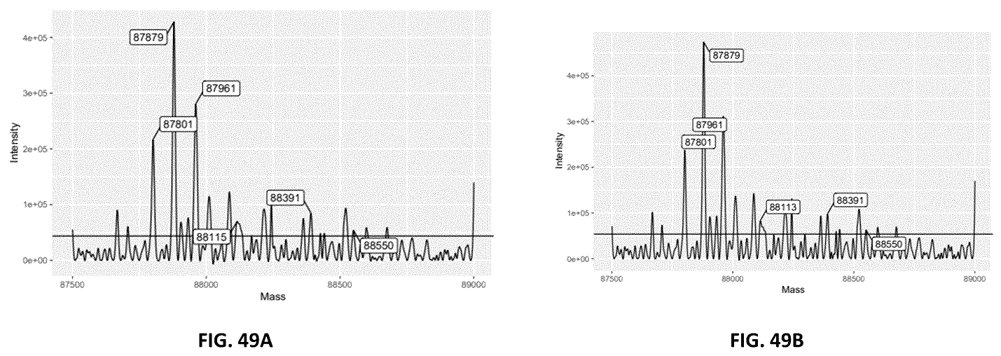

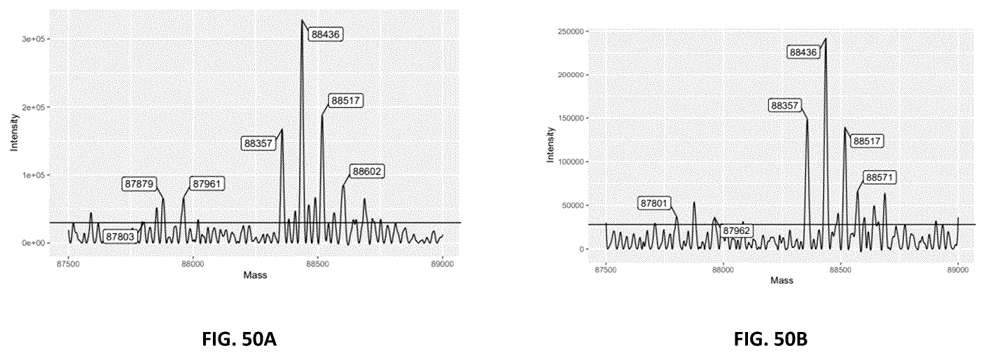

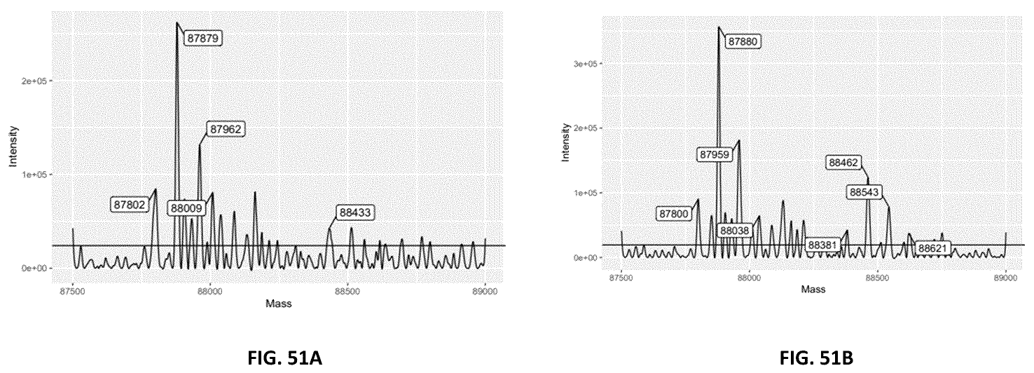

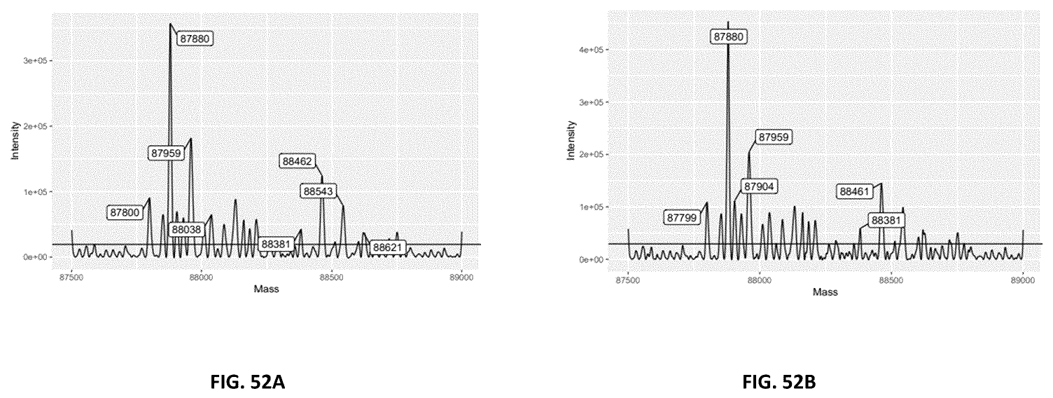

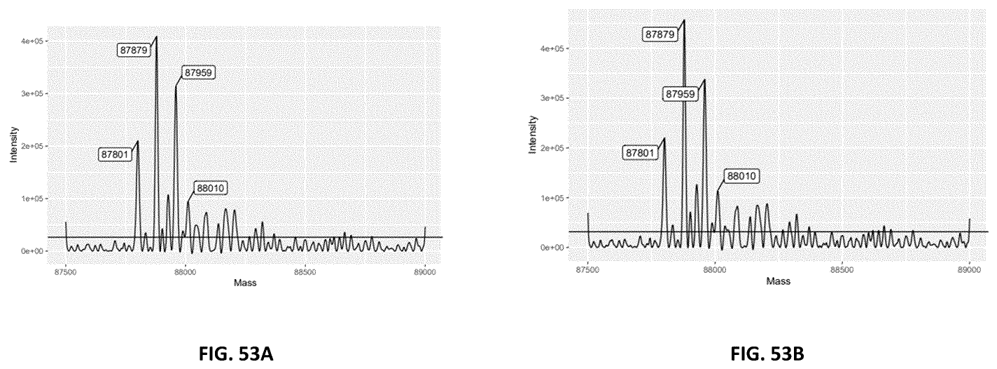

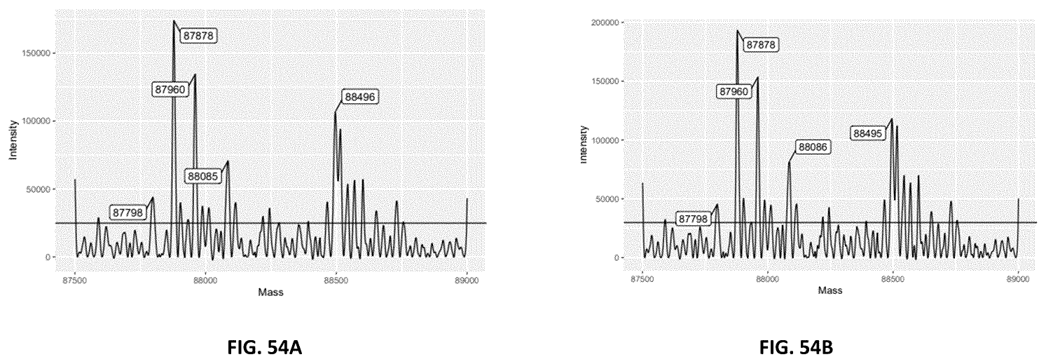

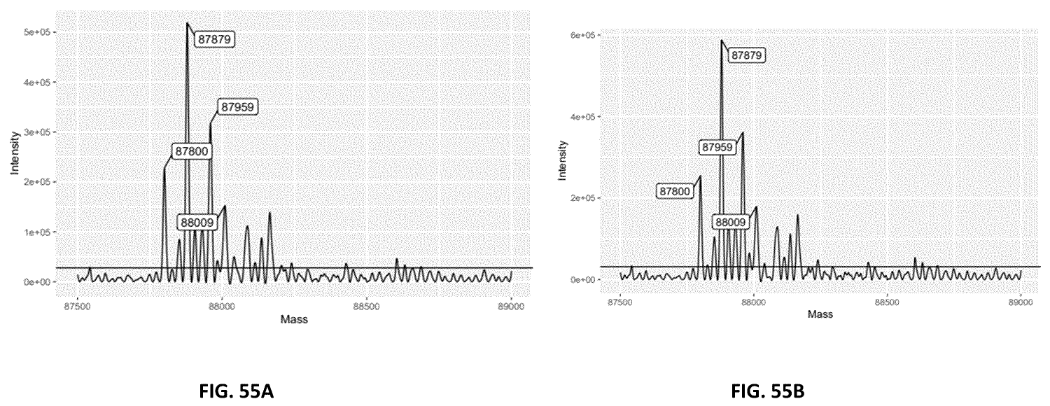

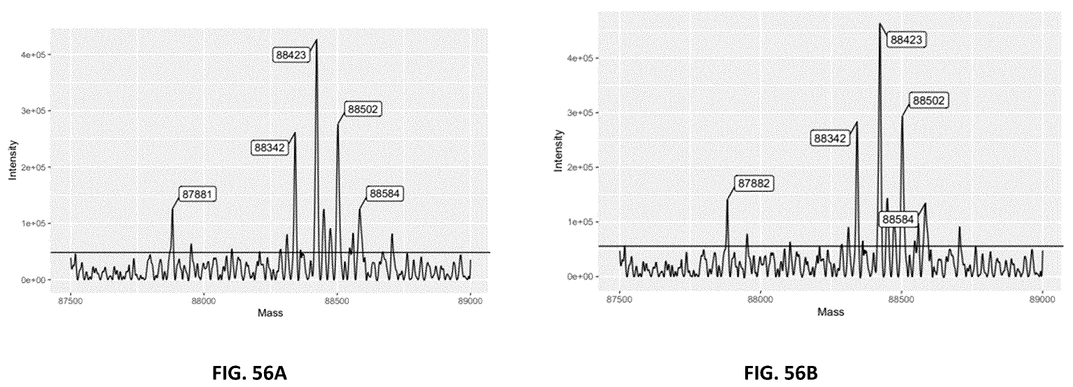

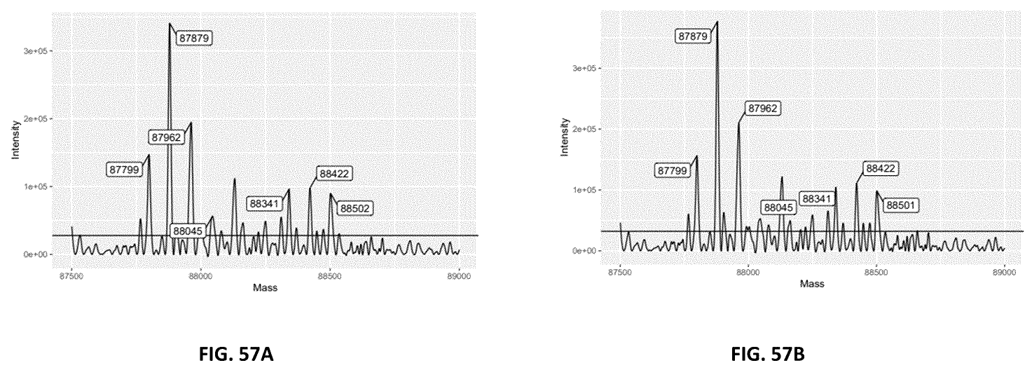

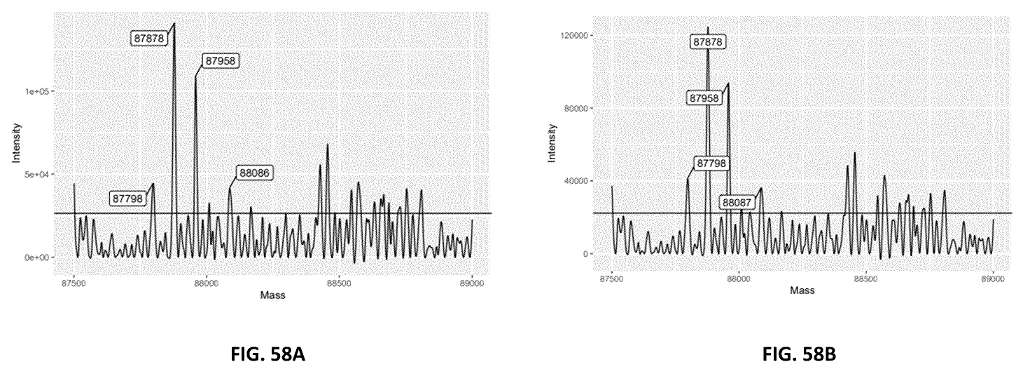

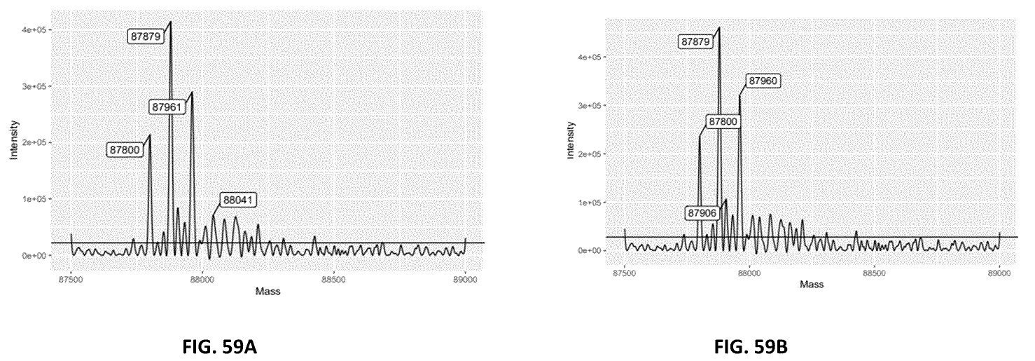

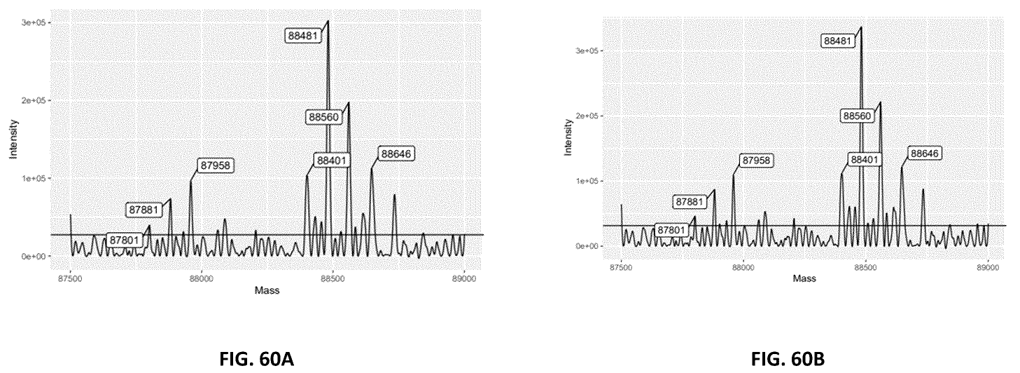

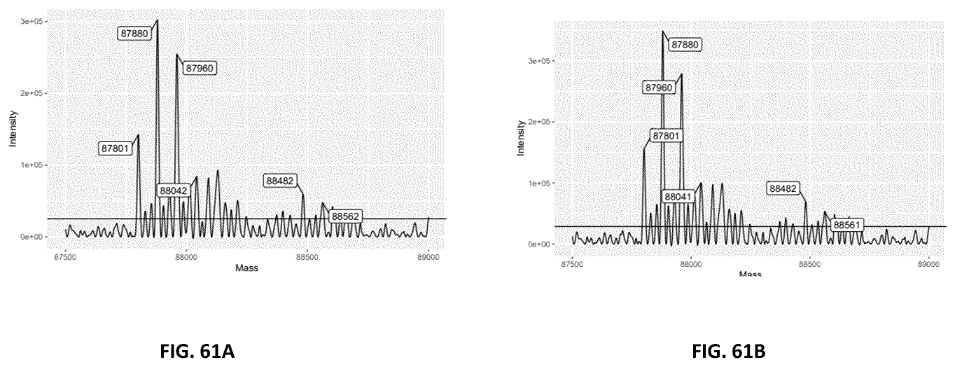

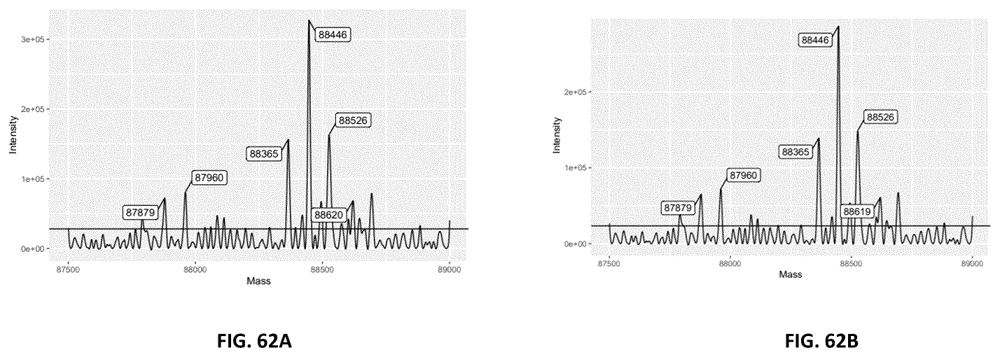

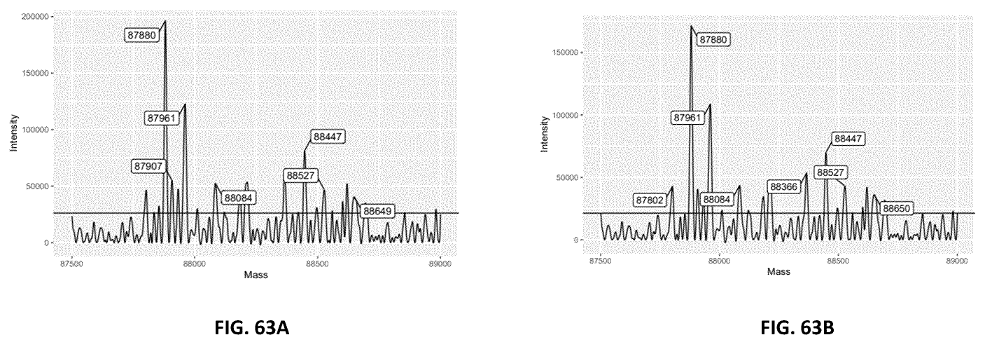

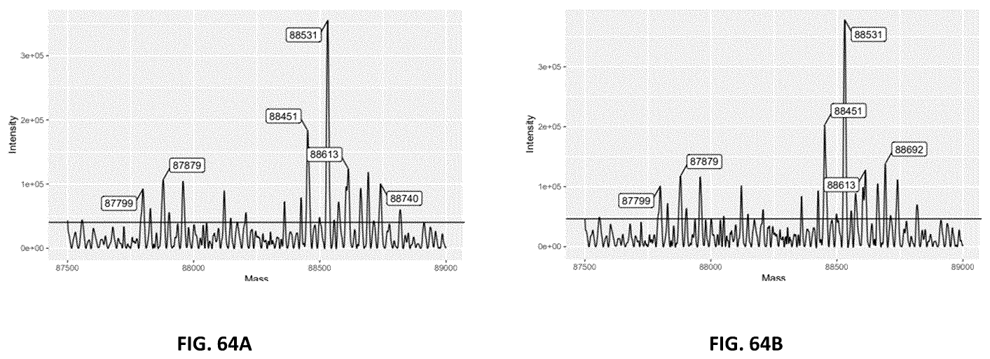

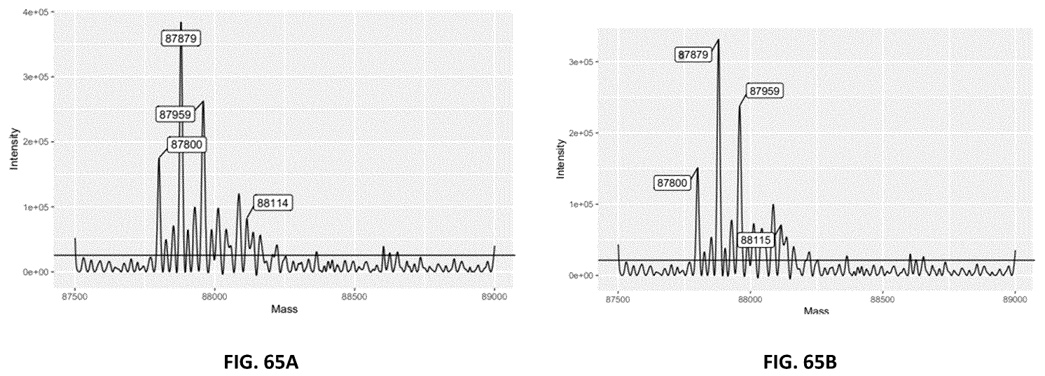

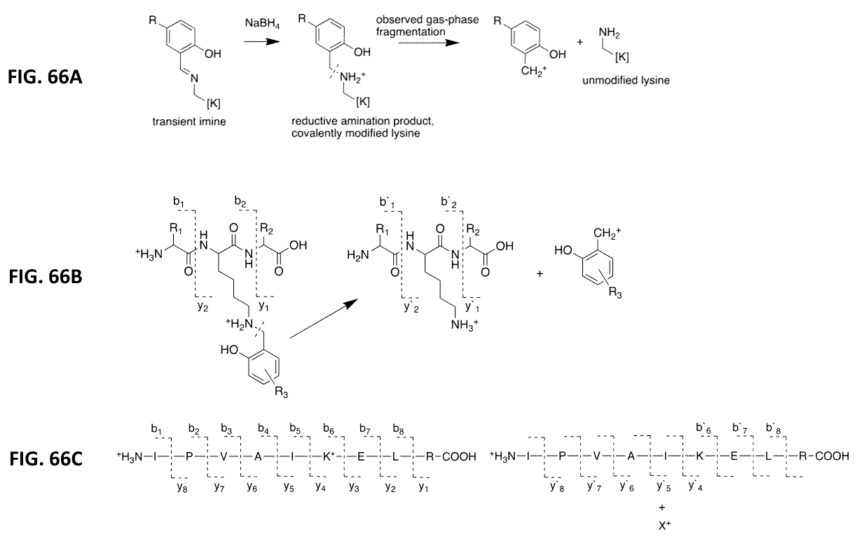

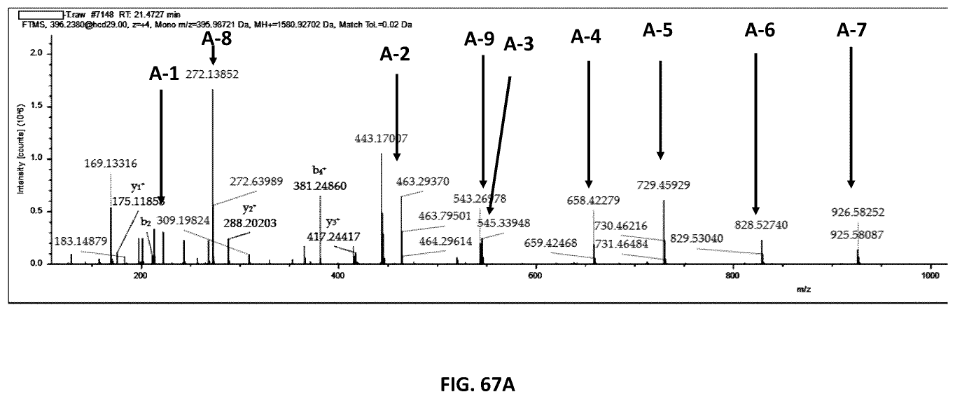

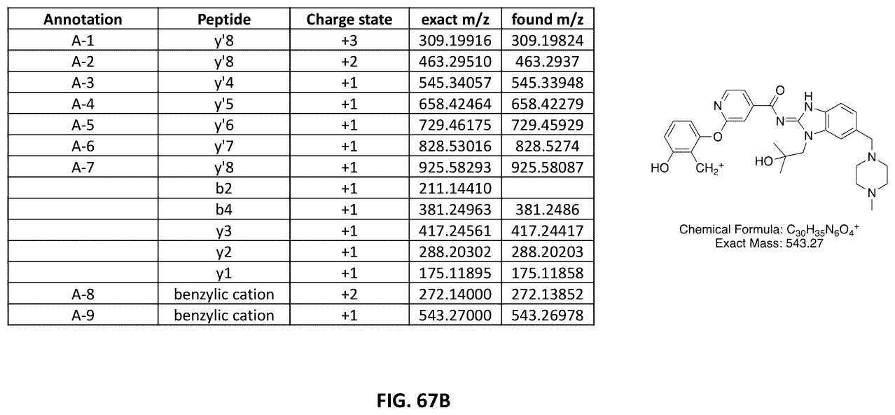

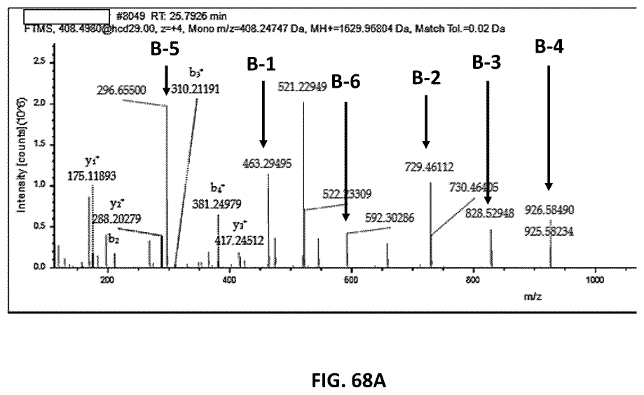

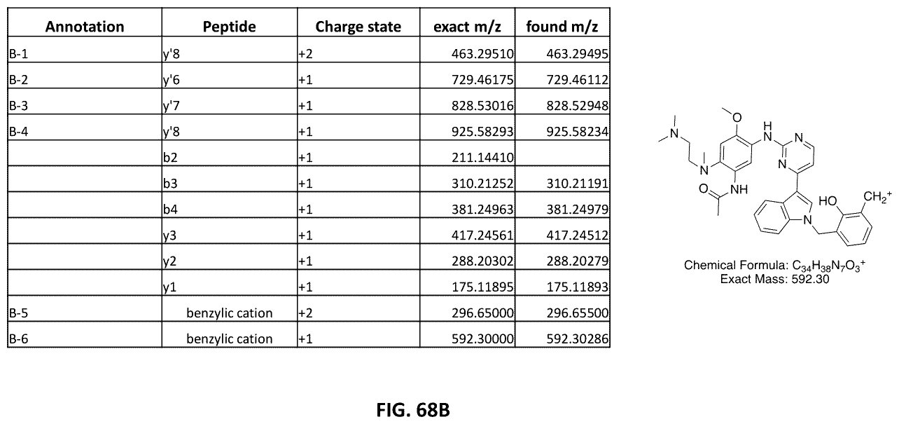

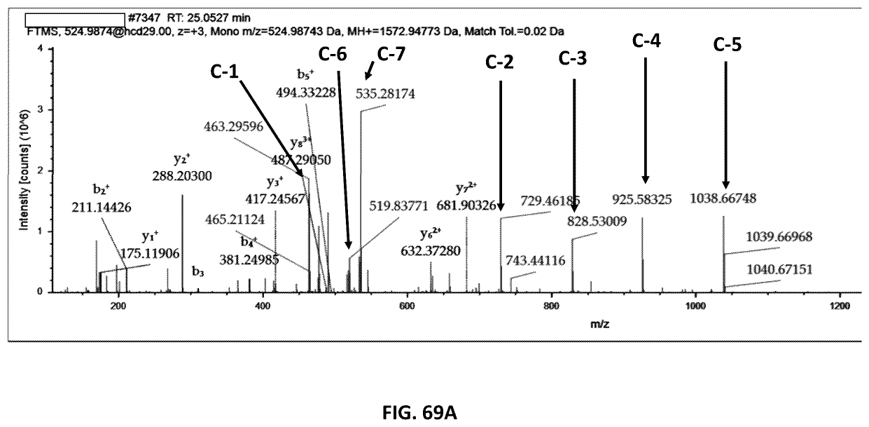

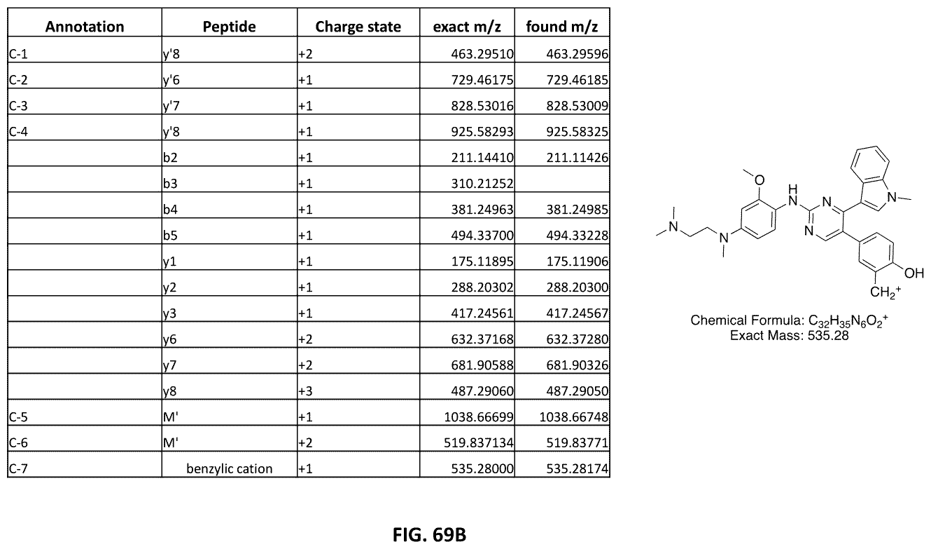

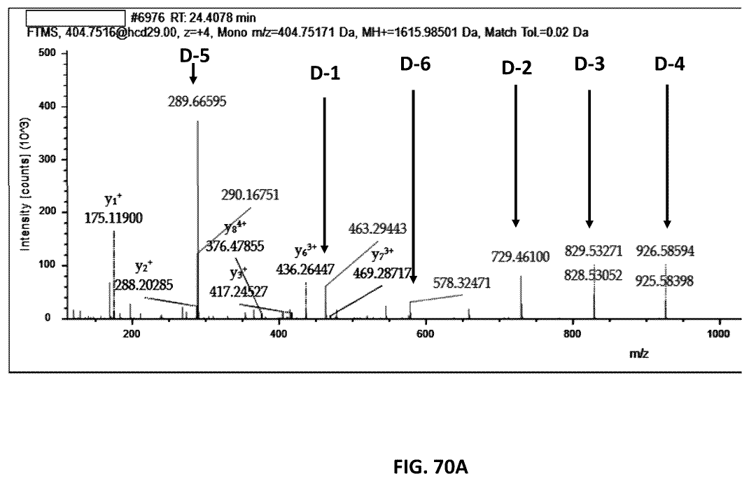

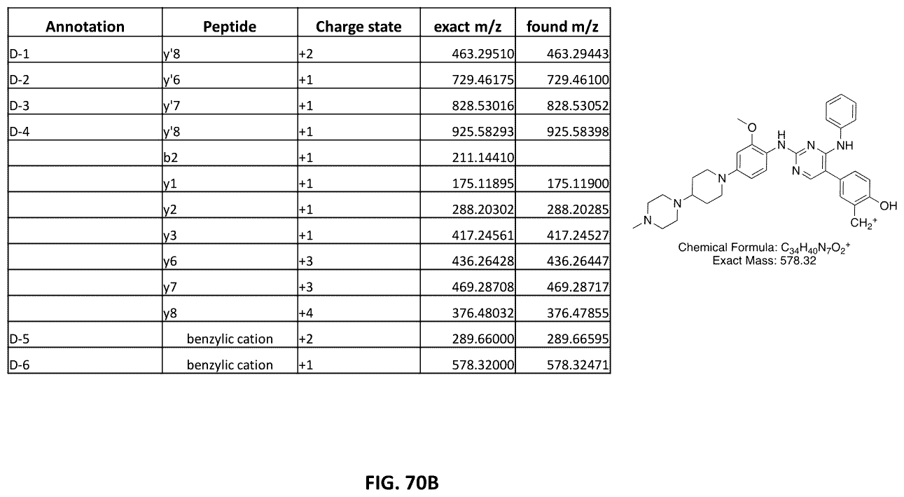

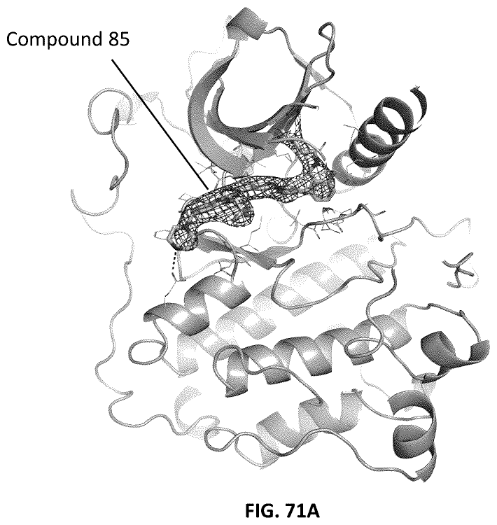

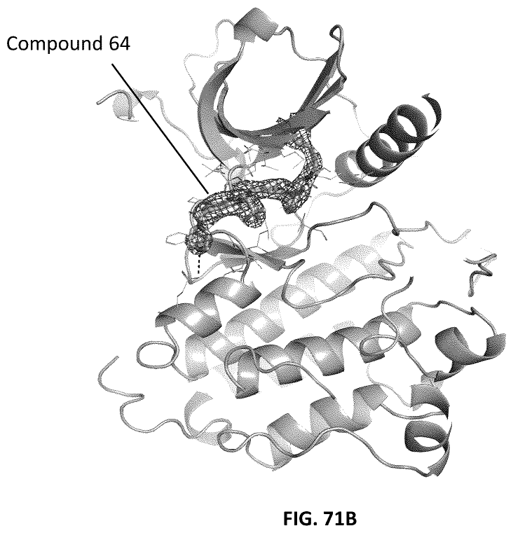

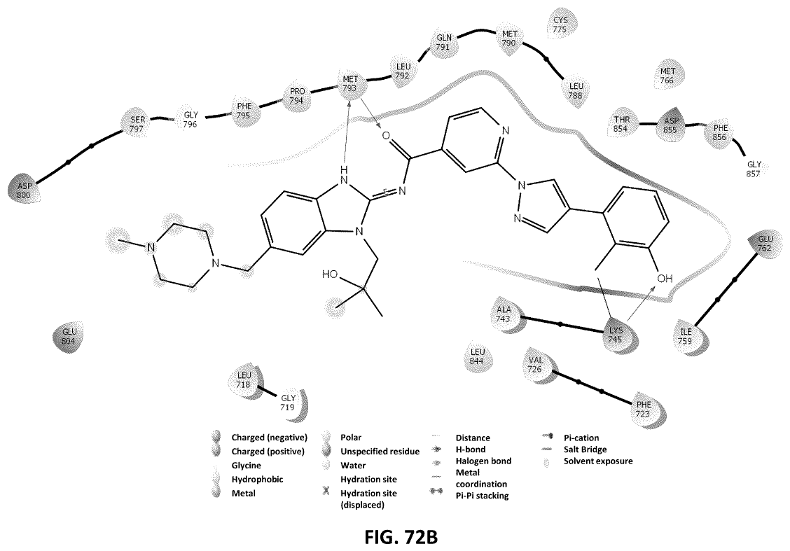

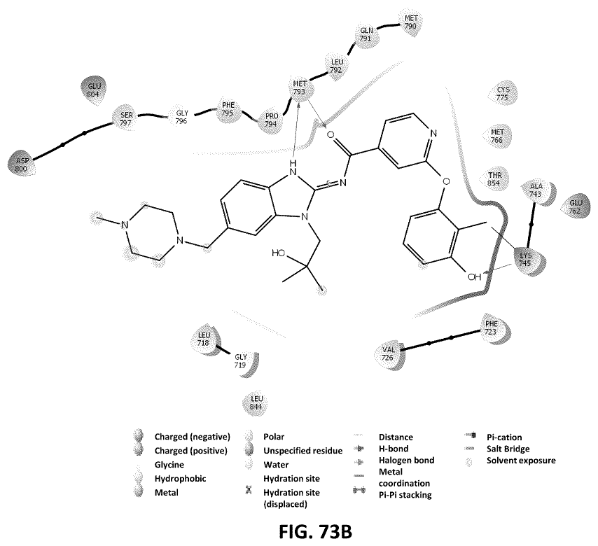

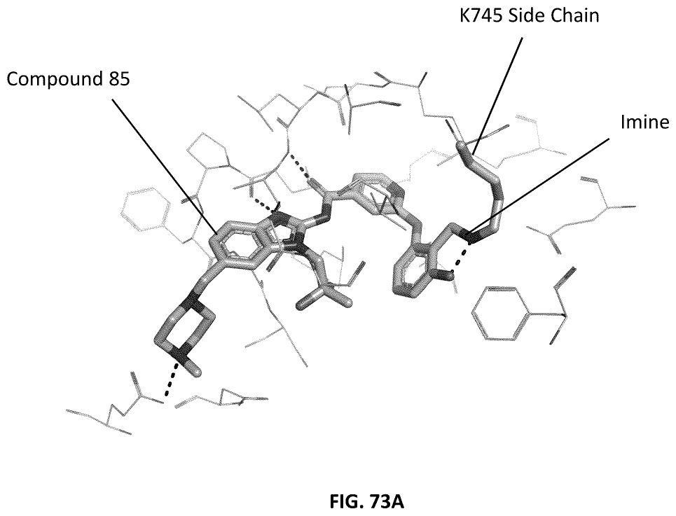

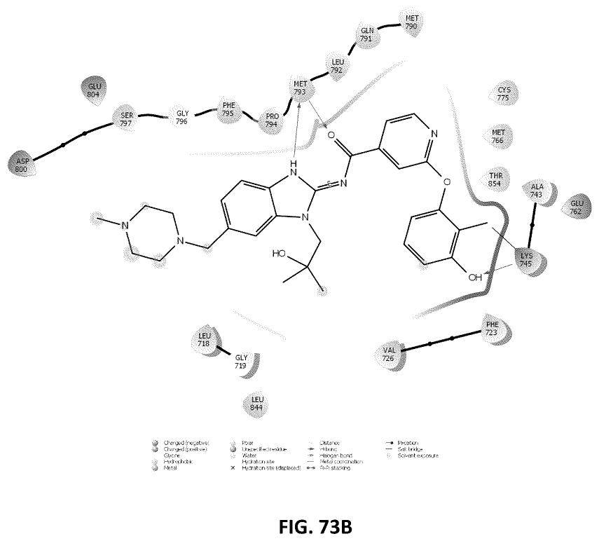

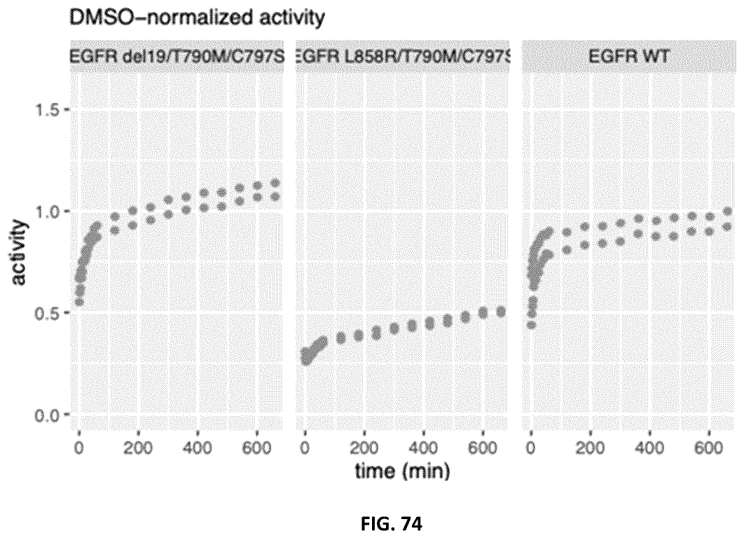

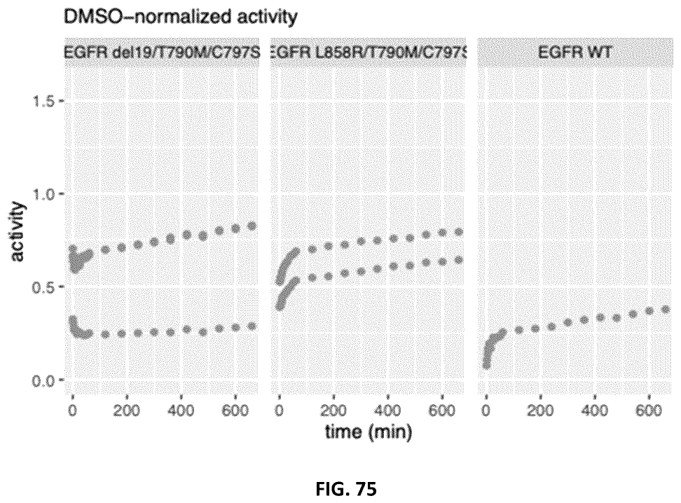

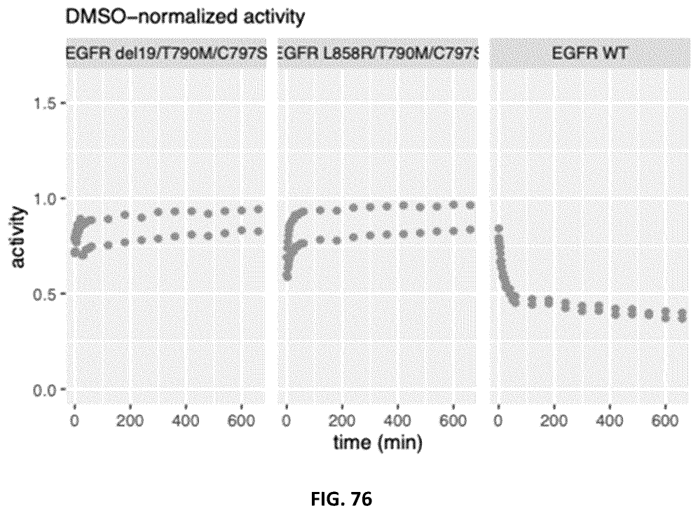

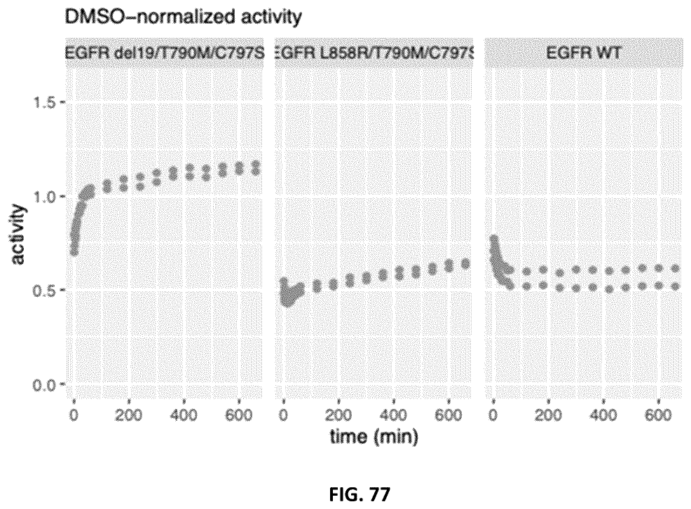

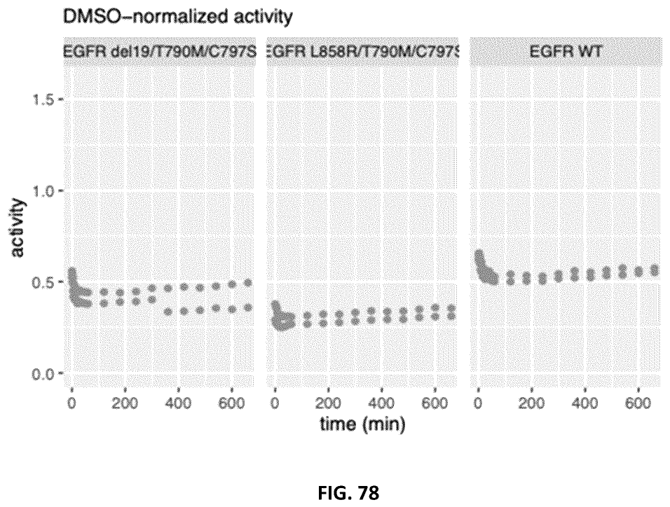

FIGS. 14-15 provide intact mass spectra following incubation of a compound of the present disclosure, both in the absence of any competitor molecule BI-4020 (FIG. 14) and in the presence of a significant molar excess of competitor molecule BI-4020 (FIG. 15).

FIGS. 16-17 provide intact mass spectra following incubation of Compound 6, both in the absence of any competitor molecule BI-4020 (FIG. 16) and in the presence of a significant molar excess of competitor molecule BI-4020 (FIG. 17). Each experiment was run in duplicate and the figures show data from each run.

FIGS. 18-19 provide intact mass spectra following incubation of Compound 26, with and without a significant molar excess of competitor molecule BI-4020. The increased intact mass observed in FIG. 18 relative to FIG. 19 corresponds to the covalent complex formed between the protein and Compound 26. In FIGS. 18 and 19, the peaks corresponding to mass values of 87800, 87879, and 87960 represent three different phosphorylation states of unmodified EGFR. In FIG. 19, and additional set of peaks is observed (m/z=88410, 88491, 88571, and 88652), corresponding to different phosphorylation states of an EGFR protein covalently bound to Compound 26. These peaks were not observed in FIG. 19, indicating the presence of a molar excess of the competitive EGFR inhibitor prevents binding of Compound 26 to EGFR and subsequent covalent complex formation.

FIGS. 20-21 provide intact mass spectra following incubation of Compound 127, with and without a significant molar excess of competitor molecule BI-4020. Taken together, these data demonstrate covalent complex formation between EGFR and Compound 127.

FIGS. 22-23 provide intact mass spectra following incubation of Compound 133, with and without a significant molar excess of competitor molecule BI-4020. Taken together, these data demonstrate covalent complex formation between EGFR and Compound 133.

FIGS. 24-25 provide intact mass spectra following incubation of Compound 7, with and without a significant molar excess of competitor molecule BI-4020. These data show that some covalent complex was formed in the absence of competitor molecule BI-4020.

FIGS. 26-27 provide intact mass spectra following incubation of Compound 11, with and without a significant molar excess of competitor molecule BI-4020. Taken together, these data demonstrate covalent complex formation between EGFR and Compound 11.

FIGS. 28-29 provide intact mass spectra following incubation of Compound 17, with and without a significant molar excess of competitor molecule BI-4020. Taken together, these data demonstrate covalent complex formation between EGFR and Compound 17.

FIGS. 30-31 provide intact mass spectra following incubation of Compound 19, with and without a significant molar excess of competitor molecule BI-4020. Taken together, these data demonstrate covalent complex formation between EGFR and Compound 19.

FIGS. 32-33 provide intact mass spectra following incubation of Compound 32, with and without a significant molar excess of competitor molecule BI-4020. Taken together, these data demonstrate covalent complex formation between EGFR and Compound 32.

FIGS. 34-35 provide intact mass spectra following incubation of Compound 62, with and without a significant molar excess of competitor molecule BI-4020. Taken together, these data demonstrate covalent complex formation between EGFR and Compound 62.

FIGS. 36-37 provide intact mass spectra following incubation of Compound 42, with and without a significant molar excess of competitor molecule BI-4020. Taken together, these data demonstrate covalent complex formation between EGFR and Compound 42.

FIGS. 38-39 provide intact mass spectra following incubation of Compound 85, with and without a significant molar excess of competitor molecule BI-4020. Taken together, these data demonstrate covalent complex formation between EGFR and Compound 85.

FIGS. 40-41 provide intact mass spectra following incubation of Compound 64, with and without a significant molar excess of competitor molecule BI-4020. Taken together, these data demonstrate covalent complex formation between EGFR and Compound 64.

FIGS. 42-43 provide intact mass spectra following incubation of Compound 57, with and without a significant molar excess of competitor molecule BI-4020. Taken together, these data demonstrate covalent complex formation between EGFR and Compound 57.

FIGS. 44-45 provide intact mass spectra following incubation of Compound 32, with and without a significant molar excess of competitor molecule BI-4020. Taken together, these data demonstrate covalent complex formation between EGFR and Compound 32.

FIGS. 46-47 provide intact mass spectra following incubation of Compound 41, with and without a significant molar excess of competitor molecule BI-4020. Taken together, these data demonstrate covalent complex formation between EGFR and Compound 41.

FIGS. 48-49 provide intact mass spectra following incubation of Compound 83, with and without a significant molar excess of competitor molecule BI-4020. Taken together, these data demonstrate covalent complex formation between EGFR and Compound 83.

FIGS. 50-51 provide intact mass spectra following incubation of Compound 79, with and without a significant molar excess of competitor molecule BI-4020. Taken together, these data demonstrate covalent complex formation between EGFR and Compound 79.

FIGS. 52-53 provide intact mass spectra following incubation of Compound 44, with and without a significant molar excess of competitor molecule BI-4020. Taken together, these data demonstrate covalent complex formation between EGFR and Compound 44.

FIGS. 54-55 provide intact mass spectra following incubation of Compound 87, with and without a significant molar excess of competitor molecule BI-4020. Taken together, these data demonstrate covalent complex formation between EGFR and Compound 87.

FIGS. 56-57 provide intact mass spectra following incubation of Compound 52, with and without a significant molar excess of competitor molecule BI-4020. Taken together, these data demonstrate covalent complex formation between EGFR and Compound 52.

FIGS. 58-59 provide intact mass spectra following incubation of Compound 84, with and without a significant molar excess of competitor molecule BI-4020. Taken together, these data demonstrate covalent complex formation between EGFR and Compound 84.

FIGS. 60-61 provide intact mass spectra following incubation of Compound 126, with and without a significant molar excess of competitor molecule BI-4020. Taken together, these data demonstrate covalent complex formation between EGFR and Compound 126.

FIGS. 62-63 provide intact mass spectra following incubation of Compound 51, with and without a significant molar excess of competitor molecule BI-4020. Taken together, these data demonstrate covalent complex formation between EGFR and Compound 51.

FIGS. 64-65 provide intact mass spectra following incubation of Compound 125, with and without a significant molar excess of competitor molecule BI-4020. Taken together, these data demonstrate covalent complex formation between EGFR and Compound 125.

In some of the preceding examples, the intact mass spectra include mass peaks corresponding to the unmodified EGFR protein. In some of such cases, these peaks may be formed by fragmentation of the covalent complex at the benzyl imine, regenerating the unmodified protein.

In view of the preceding intact mass spectra, it was demonstrated that compounds of the present disclosure readily form reversible covalent complexes with EGFR proteins.

### Example 6: Identification of Covalent Modification Site by MS/MS

### Identification of Site of Covalent Modification by Tandem Mass Spectrometry

Recombinant EGFR L858R/C₇₉₇S/T790M kinase domain residues 668-1210 (Biortus, 2.2 μM) was incubated with excess compound (4.4 μM) for 30 minutes and then treated with NaBH4 (5 mM, 5 min at room temperature) to capture reversible imines through reductive amination. Reduction reactions were quenched with 1% formic acid. The labelled protein was denatured with 8M urea, reduced and alkylated with TCEP/iodoacetamine, and digested with trypsin according to standard procedures. The resulting peptides were desalted and analyzed by tandem mass spectrometry. MS/MS spectra were searched using a database comprised of the peptide sequence for the recombinant EGFR and common laboratory contaminant proteins, along with the reverse sequences as decoy. In addition to typical variable modifications (cystine alkylation, methionine oxidation), a variable modification on lysine corresponding to compound addition (minus 0, the product of reductive amination) was allowed. Gas phase fragmentation of the compound-modified lysine benzylic amine bond was expected [citations below]. Therefore, MS/MS spectra were additionally manually annotated to account for ions generated by benzylic amine fragmentation.

The following references describe fragmentation of covalent lysine complexes, each of which is incorporated by reference herein in its entirety: (1) Simon, E. S., Papoulias, P. G. & Andrews, P. C. Selective collision-induced fragmentation of ortho-hydroxybenzyl-aminated lysyl-containing tryptic peptides. Rapid Commun Mass Sp 27, 1619-1630 (2013); (2) Simon, E. S., Papoulias, P. G. & Andrews, P. C. Substituent effects on the gas-phase fragmentation reactions of protonated peptides containing benzylamine-derivatized lysyl residues. Rapid Commun Mass Sp 26, 631-638 (2012); and (3) Simon, E. S., Papoulias, P. G. & Andrews, P. C. Gas-phase fragmentation characteristics of benzyl-aminated lysyl-containing tryptic peptides. J Am Soc Mass Spectr 21, 1624-1632 (2010).

FIG. 66A depicts sodium borohydride reduction of the reversible covalent complex between EGFR and a compound of the present disclosure (left). This process forms a benzylic amine linkage between the EGFR protein and the compound of the present disclosure. FIG. 66A further depicts that gas-phase fragmentation of the benzylic amine leads to formations of ions corresponding to (1) the unmodified lysine (or peptide comprising this lysine) and (2) a benzylic cation of the compound previously bound to the lysine (right).

FIG. 66B provides a schematic for gas phase peptide fragmentation under HCD tandem mass spectrometry, including benzylic amine fragmentation which produces a second set of b/y ions, labeled b′/y′, along with the benzylic cation derived from a compound of the present disclosure (right).

FIG. 66C depicts the nonapeptide sequence containing EGFR residue K745 produced by tryptic digestion NH3+-I-P-V-A-I-K*-E-L-R—COOH (SEQ ID NO: 1), where K* indicates lysine covalently modified by a compound of the present disclosure, and labels standard b/y fragmentation ions (left) along with double-fragmentation b′/y′ ions (right) expected during HCD fragmentation. Thus, simultaneous gas phase detection of (a) standard b/y peptide fragments, (b) double-fragmentation b′/y′ ions, and (c) the benzylic cation of a compound of the present disclosure are evidence of a reversible covalent complex formed between the compound of the present disclosure and K745 of EGFR.

FIG. 67A depicts a mass spectrum observed after the incubation of Compound 64 with EGFR, followed by sodium borohydride reduction, trypsin digest, and MS analysis. FIG. 67B provides a table of observed mass values corresponding to (a) classical b/y ions annotated during automated spectral matching (b) b′/y′ peptide fragments (peaks labeled A-1 through A-7), and (c) masses corresponding to the benzylic cation fragment of Compound 64 (peaks labeled A-8 and A-9). As discussed above, the simultaneous observation of these m z values demonstrate the formation of a covalent complex between Compound 64 and K745 of EGFR.

FIG. 68A depicts a mass spectrum observed after the incubation of Compound 7 with EGFR, followed by sodium borohydride reduction, trypsin digest, and MS analysis. FIG. 68B provides a table of observed mass values corresponding to (a) classical b/y ions annotated during automated spectral matching (b) b′/y′ peptide fragments (peaks labeled B-1 through B-4), and (c) masses corresponding to the benzylic cation fragment of Compound 7 (peaks labeled B-5 and B-6). As discussed above, the simultaneous observation of these m z values demonstrate the formation of a covalent complex between Compound 7 and K745 of EGFR.

FIG. 69A depicts a mass spectrum observed after the incubation of Compound 8 with EGFR, followed by sodium borohydride reduction, trypsin digest, and MS analysis. FIG. 69B provides a table of observed mass values corresponding to (a) classical b/y ions annotated during automated spectral matching (b) b′/y′ peptide fragments (peaks labeled C-1 through C-6), and (c) mass corresponding to the benzylic cation fragment of Compound 8 (peak labeled C-7). As discussed above, the simultaneous observation of these m z values demonstrate the formation of a covalent complex between Compound 8 and K745 of EGFR.

FIG. 70A depicts a mass spectrum observed after the incubation of Compound 3 with EGFR, followed by sodium borohydride reduction, trypsin digest, and MS analysis. FIG. 70B provides a table of observed mass values corresponding to (a) classical b/y ions annotated during automated spectral matching (b) b′/y′ peptide fragments (peaks labeled D-1 through D-4), and (b) masses corresponding to the benzylic cation fragment of Compound 3 (peaks labeled D-5 and D-6). As discussed above, the simultaneous observation of these m z values demonstrate the formation of a covalent complex between Compound 3 and K745 of EGFR.

Thus, the preceding examples demonstrate that compounds of the present disclosure form reversible covalent complexes via imine bonds of K745 of EGFR. This observation is consistent with reports that K745 is involved in ATP binding in EGFR and indicates that compounds of the present disclosure can inhibit ATP binding to EGFR by covalently engaging K745 in the ATP-binding pocket.

### Example 7: Crystallography Studies Showing Covalent Complex Formation

### Protein Expression and Purification

The EGFR 695-1022 mutant T790M, C₇₉₇S, L858R, E865 Å, E866 Å, K867A with the N-terminal affinity His-tag followed by a cleavable TEV-tag was expressed in Sf21 insect cells using baculovirus-insect cell expression system, which were infected in 2 million per milliliter at 300 K. The cells were harvested after an incubation time of 48 hours at 300 K and stored at 193 K until lysis.

The cells were lysed in buffer A (50 mM Tris/HCl, 300 mM NaCl, 20 mM imidazole, 5 mM MgCl2, 10% glycerol, 1 mM TCEP, pH 8.0) supplemented with cOmplete EDTA-free protease inhibitor tablet (Roche) using a Microfluidizer LM10. Purification was performed by N1-NTA capture of the His-tagged protein in buffer A. EGFR was eluted from the IMAC resin with 300 mM Imidazole. The His tag was cleaved by TEV protease and removed from the sample together with uncleaved protein by a reverse IMAC purification step in buffer A. As final purification step, EGFR was polished by size-exclusion chromatography with buffer B as running and storage buffer (25 mM Tris/HCl, 150 mM NaCl, 10% glycerol, 5 mM DTT, pH 8.0). The protein was then concentrated to 10 mg/mL using an Amicon concentrator at 277 K, centrifugated, aliquoted, and stored at 193 K until crystallization.

### Crystallization, Data Collection and Structure Determination

EGFR 695-1022 (T790M, C797S, L858R, E865 Å, E866 Å, K867 Å) was incubated with 1 mM Compound 85 or Compound 64 prior to co-crystallization trials. Co-crystals of EGFR/Compound 64 were obtained at 293K from sitting drops using reservoir solutions containing 1.80 M Ammonium Sulfate, 0.10 M HEPES-NaOH pH 7.20, 2% w/v PEG 2000 MME. Co-crystals of EGFR/Compound 85 were obtained at 293K from sitting drops using reservoir solutions containing 2.00 M Ammonium sulfate, 0.10 M HEPES-NaOH pH 7.50, 2% v/v PEG 550 MME. Co-crystals were flash-cooled in liquid nitrogen after addition of 20% ethylene glycol supplemented to the mother liquor. Diffraction data of Compound 85 was obtained at 100 K at beamline P11 in PETRA III, using an EIGER 2×16M detector (DECTRIS Ltd.). Diffraction data of Compound 64 was obtained at 100K at beamline ID30 Å-1 in ESRF using a PILATUS3 2M detector (DECTRIS Ltd.). These datasets were further processed with autoPROC (Global Phasing). The initial structures were determined through molecular replacement with Phaser in CCP4 suite with the structure of PDB ID 6LUD as a search model. A chemical restraint dictionary was generated using ACEDRG and CCP4i2. The models were manually adjusted using Coot, refined with REFMAC5 to a final resolution of 3.1 Å for Compound 85 complex and 2.6 Å for the Compound 64 complex.

TABLE 6
Data collection and refinement statistics

| | Compound 85 | | Compound 64 | |
|---|---|---|---|---|
| Wavelength | 1.033 | | 1 | |
| Resolution range | 33.22-3.101 | (3.211- | 30.76-2.6 | (2.693-2.6) |
| Space group | 1 2 3 | | 1 2 3 | |
| Unit cell | 148.567 148.567 148.567 90 | | 144.273 144.273 144.273 90 | |
| Total reflections | 85453 | (9168) | 70150 | (5755) |
| Unique reflections | 10069 | (1007) | 14647 | (1289) |
| Multiplicity | 8.5 | (9.1) | 4.8 | (4.5) |
| Completeness (%) | 99.58 | (98.02) | 94.00 | (82.69) |
| Mean I/sigma(I) | 7.34 | (0.83) | 7.15 | (0.71) |
| Wilson B-factor | 105.94 | | 72.37 | |
| R-merge | 0.2956 | (5.745) | 0.1351 | (2.584) |
| R-meas | 0.3147 | (6.086) | 0.1513 | (2.908) |
| R-pim | 0.1066 | (1.994) | 0.06525 | (1.3) |
| CC1/2 | 0.997 | (0.277) | 0.974 | (0.263) |
| CC* | 0.999 | (0.659) | 0.993 | (0.645) |
| Reflections used in | 10041 | (989) | 14595 | (1271) |
| Reflections used for R-free | 527 | (60) | 702 | (62) |
| R-work | 0.2091 | (0.3689) | 0.1940 | (0.3771) |
| R-free | 0.2701 | (0.4407) | 0.2312 | (0.3390) |
| CC(work) | 0.955 | (0.431) | 0.963 | (0.256) |
| CC(free) | 0.987 | (0.295) | 0.947 | (0.392) |

| | | |
|---|---|---|
| Number of non-hydrogen | 2690 | 2560 |
| macromolecules | 2609 | 2454 |
| ligands | 57 | 68 |
| solvent | 24 | 38 |
| Protein residues | 325 | 307 |
| RMS(bonds) | 0.016 | 0.010 |
| RMS(angles) | 1.63 | 1.71 |
| Ramachandran favored (%) | 90.40 | 94.06 |
| Ramachandran allowed (%) | 8.67 | 4.95 |
| Ramachandran outliers (%) | 0.93 | 0.99 |
| Clashscore | 12.61 | 8.56 |
| Average B-factor | 116.99 | 80.41 |
| macromolecules | 116.98 | 80.21 |
| ligands | 129.71 | 97.47 |
| solvent | 88.55 | 62.57 |

Statistics for the highest-resolution shell are shown in parentheses.

FIG. 71A depicts the electron density map of Compound 85 co-crystalized with EGFR protein (shown in ribbon structure). The crystal structure shows Compound 85 bound to the ATP binding site and forming a covalent bond with K745 of EGFR.

FIG. 71B depicts the electron density map of Compound 64 co-crystalized with EGFR protein (shown in ribbon structure). The crystal structure shows Compound 64 bound to the ATP binding site and forming a covalent bond with K745 of EGFR.

FIG. 72A is a close-up from the crystal structures depicted in FIG. 71A (co-crystallization of EGFR and Compound 85). In FIG. 72A, Compound 85 and the side chain of K745 of EGFR are represented by thicker stick model and the adjacent residues of EGFR are represented by a thinner stick model. Hydrogen bonds are depicted by dashed lines. FIG. 72A shows the imine bond formed between the nitrogen of K745 and the aldehyde of Compound 85, as well as the hydrogen bond between the imine nitrogen lone pair and the adjacent phenol hydrogen. The crystal structure provided in FIG. 71A thereby depicts the formation of a covalent complex between Compound 85 and K745 of EGFR.

FIG. 72B provides a 2-D diagram detailing the interactions between Compound 85 and residues of the EGFR protein. The imine bond is depicted by the line between K745 and the methylene of Compound 85. The hydrogen bond between the imine of K745 and the phenol of Compound 85 is depicted by the adjacent arrow. In addition to the covalent bond between K745 and Compound 85, additional hydrogen bonds are identified, including those between Met 793 and the benzimidazole core N—H, as well as the adjacent carbonyl on Compound 85. Further, Van der Waals interactions between the binding pocket residues and Compound 85 are also depicted.

FIG. 73A is a close-up from the crystal structures depicted in FIG. 71B (co-crystallization of EGFR and Compound 64). In FIG. 72A, Compound 64 and the side chain of K745 of EGFR are represented by thicker stick model and the adjacent residues of EGFR are represented by a thinner stick model. Hydrogen bonds are depicted by dashed lines. FIG. 73A shows the imine bond formed between the nitrogen of K745 and the aldehyde of Compound 64, as well as the hydrogen bond between the imine nitrogen lone pair and the adjacent phenol hydrogen. The crystal structure provided in FIG. 73A thereby depicts the formation of a covalent complex between Compound 64 and K745 of EGFR.

FIG. 73B provides a 2-D diagram detailing the interactions between Compound 64 and residues of the EGFR protein. The imine bond is depicted by the line between K745 and the methylene of Compound 64. The hydrogen bond between the imine of K745 and the phenol of Compound 64 is depicted by the adjacent arrow. In addition to the covalent bond between K745 and Compound 64, additional hydrogen bonds are identified, including those between Met 793 and the benzimidazole core N—H, as well as the adjacent carbonyl on Compound 64. Further, Van der Waals interactions between the binding pocket residues and Compound 64 are also depicted.

Thus, the foregoing Examples provides evidence of covalent complex formation between EGFR and compounds of the present disclosure.

### Example 8. EGFR Jump Dilution Studies

Enzyme:inhibitor complexes were pre-formed at EGFR 100Y¹ final reaction concentration by incubating enzyme with an inhibitor at the greater concentration of either 10Y¹ inhibitor IC50, or 1:1.2 stoichiometry with enzyme. The reaction buffer composition was 50 mM HEPES pH 7.4, 10 mM MgCl_{2-, 1}% glycerol, 0.0125% Brij-35, 1.2 mM DTT, and 0.02% BSA.

Table 7 provides details of the reagents and concentrations used in these dilution studies.

TABLE 7
Reagents and Dilution Parameters

| | | | Enzyme | Final enzyme |
|---|---|---|---|---|
| | | | concentration | concentration, |
| Protein | Supplier | Cat No. | (preform), nM | nM |
| EGFR WT | SignalChem | E10-112G | 2000 | 20 |
| EGFR (d746-750) | SignalChem | E10-122UG | 500 | 5 |
| T790M C797S | | | | |
| EGFR (L858R | SignalChem | E10-122VG | 250 | 2.5 |
| T790M C797S) | | | | |

After incubating enzyme:inhibitor complexes on ice for 30 min, reactions were diluted in two steps 100-fold into reaction buffer supplemented with 1 mM ATP, and 20 uM AssayQuant substrate (AQT0734), with final reaction volumes of 20 μL in a 384 well reaction plate. Wells were read immediately and at timepoints post-dilution for up to 10 hrs in a PerkinElmer Ensight plate reader, according to the AssayQuant manufacturer's instructions.

Data processing: Raw fluorescence signal was first background corrected by subtracting the fluorescence intensity of wells containing only the AssayQuant substrate, and no enzyme. Enzyme activity was normalized relative DMSO controls for each enzyme at each timepoint. In this way, DMSO-treated wells appear as a constant normalized activity=1 throughout the assay.

FIGS. 74-78 provide the DMSO-normalized enzyme activity for Compounds 1, 3, 6, 7 and 8, respectively, each incubated with three different EGFR proteins (EGFR de119/T790M/C₇₉₇S (left); EGFR L858R/T790M/C797S (middle); WT-EGFR (right)). The X-axis of each of the three graphs represents the time after dilution that the enzyme activity was measured, with t=0 representing the time of jump dilution. The Y-axis of each graph provides the enzymatic activity at each time point. Enzymatic activity is normalized to the DMSO-treated control samples (i.e., the same enzyme variant treated with DMSO instead of compound). As such, a DMSO-normalized value of 1.0 represents full enzymatic activity (i.e., equivalent to the DMSO-treated control), while a value below 1.0 represents reduced activity relative to control (i.e., some degree of enzyme inhibition at the time measured). The data points over time (e.g., from 0 to 600 minutes post-dilution) are therefore indicative of the off-rate for the compound. Slow-off kinetics present as a gradual, shallow upward slow of the enzyme activity over time, while rapid release of the compound from the enzyme present as a steep upward slope to a value of roughly 1.0 in a short amount of time. Thus, the jump-dilution data provided a qualitative estimate of the off-rate kinetics for each compound against the panel of EGFR variants. Experiments were run in duplicate, resulting in two different data sets for each graph.

As shown in FIG. 74, Compound 1 exhibited slow-off kinetics for EGFR L858R/T790M/C797S (middle), but not for WT-EGFR or EGFR dell9/T790M/C797S. This was seen by comparing the activity curve of WT-EGFR (right) to that of EGFR L858R/T790M/C797S (middle). For WT-EGFR enzymatic activity quickly returned following dilution, evidenced by a rapid rise in DMSO-normalized enzyme activity to 1.0 (i.e., same level of enzymatic activity for both DMSO-treated WT-EGFR control and WT-EGFR treated with Compound 1). In contrast, the activity curve for EGFR L858R/T790M/C797S (middle) shows that enzyme activity was slow to recover, with DMSO-normalized enzyme activity reaching only 0.5 over 10 hours (600 minutes) after jump dilution. This slow recovery of enzyme activity was consistent with reversible covalent modification of EGFR L858R/T790M/C797S by Compound 1. However, Compound 1 did not exhibit slow-off kinetics for EGFR del19/T790M/C797S (left), and instead the activity curve for this EGFR mutant largely mirrored that of WT-EGFR, with a rapid rise in enzyme activity following dilution.

Data for Compound 3 are provided in FIG. 75. Compound 3 exhibited slow-off kinetics for all three EGFR variants tested (i.e., EGFR del19/T790M/C797S (left); EGFR L858R/T790M/C797S (middle); WT-EGFR (right)). This was evidenced by the gradual upward slope of the data points towards 1.0 for each EGFR variant, indicating a gradual release of the inhibitors from the enzyme. Compound 3 appeared to exhibit some degree of selectivity for WT-EGFR, having a DMSO-normalized enzyme activity value of less than 0.5 even at 10 hours post-dilution, compared to EGFR L858R/T790M/C797S (middle), which exhibited enzyme activity values of roughly 0.7 at the same time point when treated with Compound 3. Although there were some differences between the two replicants for the experiments with EGFR de119/T790M/C797S (left), inhibition and slow-off kinetics were observed for both. However, the magnitude of inhibition was different between each experiment.

As shown in FIG. 76, Compound 6 exhibited slow-off kinetics for WT-EGFR only, but not for either of the two mutant EGFR variants (compare the prolonged inhibition of WT-EGFR (right) to the rapid reversion to a DMSO-normalized enzyme activity value of nearly 1.0 for both EGFR de119/T790M/C797S (left) and EGFR L858R/T790M/C797S (middle)).

Data for Compound 7 are provided in FIG. 77. Compound 7 exhibited slow-off kinetics for both WT-EGFR (right) and EGFR L858R/T790M/C797S (middle), but not for EGFR de119/T790M/C797S (left) (compare the prolonged inhibition of WT-EGFR (right) and EGFR L858R/T790M/C797S (middle) to the rapid reversion to a DMSO-normalized enzyme value of roughly 1.0 observed for EGFR dell9/T790M/C797S (left).

As demonstrated in FIG. 78, Compound 3 exhibited slow-off kinetics for all three EGFR variants tested (i.e., EGFR dell9/T790M/C797S (left); EGFR L858R/T790M/C797S (middle); WT-EGFR (right)). Further, FIG. 14 shows that Compound 3 inhibited EGFR del19/T790M/C797S (left) and EGFR L858R/T790M/C797S (middle) with some degree of selectivity over WT-EGFR (right). This selectivity was observed by comparing the DMSO-normalized enzyme activity value at 10-hours post-dilution for WT-EGFR (right, value of >0.5) to that for EGFR L858R/T790M/C797S (middle, value of roughly 0.3).

Thus, FIGS. 74 to 78 demonstrate that compounds of the present disclosure exhibit slow-off kinetics for EGFR binding, which is consistent with the formation of a reversible covalent interaction between EGFR and the compounds of the present disclosure.

### Example 9. EGFR Inhibition

### EGFR Biochemical Assays

Biochemical activity was determined using a chelation-enhanced fluorescence assay. EGFR enzyme was incubated with DMSO-dissolved inhibitors in reaction buffer (50 mM HEPES pH 7.4, 10 mM MgCl2, 1% glycerol, 0.0125% Brij-35, 1.2 mM DTT, and 0.02% BSA) for 30 min at RT before initiating reactions with AssayQuant peptide AQT0734 (10 μM) and ATP (see enzyme and ATP details in table below). Reaction times were 360 min (EGFR WT), or 150 min (EGFR (d746-750) T790M C797S; EGFR L8585R T790M C797S). Total substrate phosphorylation was measured as fluorescence intensity (excitation 360 nm, emission 480 nm) on a PerkinElmer Ensight plate reader. IC50 values were calculated by fitting background-subtracted fluorescence values to a log(inhibitor) vs. response Hill equation.

| | | | Final enzyme | ATP |
|---|---|---|---|---|
| | | Cat | concentration, | concentration, |
| Protein | Supplier | No. | nM | nM |
| EGFR WT | SignalChem | E10- | 20 | 25 |
| | | 112G | | |
| EGFR | SignalChem | E10- | 2.5 | 100 |
| (d746-750) | | 122UG | | |
| T790M | | | | |
| C797S | | | | |
| EGFR | SignalChem | E10- | 1.25 | 50 |
| (L858R | | 122VG | | |
| T790M | | | | |
| C797S) | | | | |

The results of this assay are provided below in Table 8:

TABLE 8

| | AssayQuant | AssayQuant | |
|---|---|---|---|
| | EGFR | EGFR | AssayQuant |
| Cmpd. | del19/TM/CS | LR/TM/CS | EGFR WT |
| No. | IC₅₀ (nM) | IC₅₀ (nM) | IC₅₀ (nM) |
| Ref. 1 | >1.00E+03 | >1.00E+03 | 33.4 |
| 3 | 158 | 76.9 | |
| 4 | 83 | 48.8 | >1.00E+03 |
| 5 | 18.8 | 8.09 | 398 |
| 6 | 364 | 164 | >1.00E+03 |
| 8 | 252 | 602 | >1.00E+03 |
| 9 | 35.1 | 20.9 | 4.89 |
| 11 | 267 | 22.6 | 11 |
| 12 | 679 | 674 | |
| 13 | >1.00E+03 | 290 | 94 |
| 14 | 170 | 18 | 63.4 |
| 15 | 207 | 57.9 | 19.1 |
| 16 | >1.00E+03 | >1.00E+03 | 36.4 |
| 17 | >1.00E+03 | >1.00E+03 | 98.4 |
| 18 | 175 | 11.5 | 9.08 |
| 19 | >1.00E+03 | >1.00E+03 | >1.00E+03 |
| 20 | >1.00E+03 | >1.00E+03 | >1.00E+03 |
| 21 | >1.00E+03 | >1.00E+03 | 271 |
| 22 | 98.7 | 6.01 | 211 |
| 23 | 991 | >1.00E+03 | 17.2 |
| 24 | >1.00E+03 | 21.7 | 10.8 |
| 25 | >1.00E+03 | 390 | 87.5 |
| 26 | >1.00E+03 | >1.00E+03 | 872 |
| 27 | 8.68 | 15.3 | >1.00E+03 |
| 28 | >1.00E+03 | >1.00E+03 | >1.00E+03 |
| 29 | 672 | >1.00E+03 | >1.00E+03 |
| 30 | 125 | 75.7 | 255 |
| 31 | 579 | 186 | >1.00E+03 |
| 32 | >1.00E+03 | 136 | 526 |
| 33 | 62.6 | 13.7 | 76.5 |
| 34 | 16.2 | 11.6 | 278 |
| 35 | 29.3 | 72.7 | 292 |
| 41 | 7.85 | 1.51 | 22.2 |
| 42 | 60.1 | 2.1 | 181 |
| 43 | 54.4 | 5.19 | >1.00E+03 |
| 44 | 33.7 | 8.61 | 197 |
| 45 | >1.00E+03 | 43.7 | >1.00E+03 |
| 46 | 187 | 53.2 | >1.00E+03 |
| 47 | 15.4 | 101 | >1.00E+03 |
| 48 | >1.00E+03 | 596 | >1.00E+03 |
| 49 | 797 | >1.00E+03 | >1.00E+03 |
| 50 | >1.00E+03 | >1.00E+03 | >1.00E+03 |
| 51 | 0.795 | 0.147 | 5.88 |
| 52 | 4.22 | 0.187 | 8.42 |
| 53 | 2.27 | 0.275 | 5.97 |
| 55 | 2.18 | 0.307 | 5.33 |
| 56 | 99.4 | 87.4 | 278 |
| 57 | 2.44 | 0.315 | 6.03 |
| 58 | 2.44 | 0.422 | 10.3 |
| 59 | 6 | 0.424 | 9.32 |
| 60 | 2.65 | 0.438 | 8.29 |
| 61 | 15.1 | 0.515 | 17.9 |
| 62 | 1.23 | 0.52 | 7.05 |
| 63 | 4.56 | 0.526 | 8.55 |
| 64 | 3.06 | 0.623 | 8.96 |
| 65 | 1.59 | 0.616 | 10.1 |
| 66 | 5.96 | 0.629 | 18.6 |
| 68 | 2.98 | 0.899 | 9.73 |
| 69 | 2.68 | 0.911 | 58.2 |
| 70 | 6.82 | 0.972 | 11.5 |
| 71 | 10 | 1.08 | 10.4 |
| 72 | 2.48 | 1.11 | 7.8 |
| 73 | 11 | 1.12 | 10.1 |
| 74 | 6.22 | 1.16 | 8.75 |
| 75 | 10.1 | 1.19 | 14.6 |
| 76 | 14.3 | 1.2 | 13.5 |
| 77 | 3.87 | 1.3 | 23.7 |
| 78 | 1.93 | 1.34 | 8.81 |
| 80 | 6.57 | 1.47 | 10.9 |
| 81 | 3.9 | 1.61 | 28 |
| 82 | 102 | 2.52 | 67.9 |
| 83 | 26.9 | 2.77 | 131 |
| 84 | 10.3 | 3.74 | 61 |
| 85 | 5.6 | 3.82 | 52.1 |
| 86 | 13 | 3.84 | 23.8 |
| 87 | 7.15 | 4.06 | 42 |
| 88 | 7.74 | 5.64 | 108 |
| 89 | 6.82 | 6.01 | 49.1 |
| 90 | 5.64 | 6.47 | 85.4 |
| 91 | 23.5 | 6.53 | 49.3 |
| 92 | 73 | 7.23 | 296 |
| 93 | 356 | 8.07 | 490 |
| 94 | 39.2 | 8.27 | 444 |
| 95 | 465 | 9.55 | 721 |
| 96 | 77.6 | 13 | 508 |
| 97 | 29.1 | 13.8 | 115 |
| 98 | 93.4 | 15.4 | 148 |
| 99 | 31.6 | 17.9 | 129 |
| 100 | 73.7 | 18.1 | 74.3 |
| 101 | 30.6 | 18.8 | 200 |
| 102 | 13.8 | 28.5 | 348 |
| 104 | 201 | 92.2 | 344 |
| 105 | 577 | 174 | 280 |
| 107 | 1.8 | 0.904 | 8.71 |
| 108 | 87.2 | 8.05 | 81 |
| 109 | 0.406 | 0.125 | 4.98 |
| 110 | 1.2 | 0.119 | 5.01 |
| 111 | 7.16 | 1.45 | 104 |
| 112 | >1.00E+03 | >1.00E+03 | >1.00E+03 |
| 121 | 7.37 | 1.45 | 10.5 |
| 122 | 3.49 | 1.49 | 8.24 |
| 123 | 116 | 7.42 | 221 |
| 125 | 6.11 | 7.23 | 29.4 |
| 126 | 23.8 | 7.24 | 13 |
| 127 | 17.1 | 13.8 | 38.2 |
| 130 | 226 | 26.3 | 529 |
| 131 | 275 | 27.2 | >1.00E+03 |
| 132 | >1.00E+03 | 38 | >1.00E+03 |
| 133 | 160 | 79.3 | 316 |
| 134 | 925 | 203 | >1.00E+03 |
| 135 | 420 | 261 | 80.1 |
| 137 | 591 | 738 | >1.00E+03 |

### Example 10. Cell Growth Inhibition

Ba/F3 pEGFR Assays

Cellular activity was determined using the Ba/F3 system for studying oncogenic mutant kinases (Warmuth 2007). Three isogenic Ba/F3 cell lines were generated to grow independently of IL-3 by lentiviral transduction with plasmids encoding EGFR protein, either with WT sequence or containing one set of three mutations (del746-750 T790M C797S; T790M C797S L858R). Transduced cells were maintained in RPMI 1640 with 10% FBS. EGFR WT transduced cells were grown in media supplemented with EGF. To assay cellular activity of inhibitors, cells were treated with inhibitor from 1000X DMSO stock dilutions and incubated for 2 hrs at 37° C. 5% CO₂. Subsequently, p-EGFR (Tyr1068) was quantified using a commercial sandwich immunoassay (PerkinElmer AlphaLISA SureFire assay; ALSU-PEGFR) according to the manufacturer's instructions. IC50 values were calculated by fitting background-subtracted fluorescence values to a log(inhibitor) vs. response Hill equation. An example is this protocol is found in Warmuth, M., Kim, S., Gu, X. J., Xia, G. & Adriin, F. Ba/F3 cells and their use in kinase drug discovery. Curr Opin Oncol 19, 55-60 (2007), which is incorporated herein in its entirety.

The results of this assay are provided below in Table 9:

TABLE 9

| | pEGFR Ba/F3 | pEGFR Ba/F3 | pEGFR Ba/F3 |
|---|---|---|---|
| Cmpd. | del19/T790M/C797S | L858R/T790M/C797S | WT |
| No. | IC₅₀ (nM) | IC₅₀ (nM) | IC₅₀ (nM) |
| Ref. 1 | >10.0E+03 | >10.0E+03 | 51.2 |
| 4 | 8340 | >10.0E+03 | >10.0E+03 |
| 5 | 2020 | >10.0E+03 | >10.0E+03 |
| 8 | >10.0E+03 | >10.0E+03 | >10.0E+03 |
| 9 | 868 | 600 | 42.5 |
| 11 | >7.52E+03 | 5420 | 70.8 |
| 13 | >10.0E+03 | >10.0E+03 | >10.0E+03 |
| 14 | >10.0E+03 | >10.0E+03 | >10.0E+03 |
| 15 | 6240 | 6980 | 9650 |
| 18 | 628 | 386 | |
| 20 | 5590 | >10.0E+03 | >10.0E+03 |
| 23 | 2580 | 2710 | 552 |
| 28 | >10.0E+03 | >10.0E+03 | >10.0E+03 |
| 29 | >10.0E+03 | >10.0E+03 | >10.0E+03 |
| 30 | >10.0E+03 | 4730 | |
| 33 | 3610 | 3340 | |
| 34 | 9970 | 4820 | |
| 35 | >10.0E+03 | 3090 | >10.0E+03 |
| 42 | 2740 | 1030 | >10.0E+03 |
| 43 | 6230 | 687 | |
| 44 | 745 | 600 | 6850 |
| 45 | >10.0E+03 | >10.0E+03 | >10.0E+03 |
| 46 | 2020 | 726 | |
| 48 | >10.0E+03 | >10.0E+03 | >10.0E+03 |
| 49 | >10.0E+03 | 8980 | 2290 |
| 51 | 38.9 | 36.5 | 1900 |
| 52 | 57.7 | 92.5 | 4860 |
| 53 | 93.9 | 52.2 | >8.18E+03 |
| 55 | 32.7 | 30.8 | 1430 |
| 56 | 706 | >10.0E+03 | 763 |
| 57 | 110 | 308 | 1770 |
| 58 | 460 | 405 | >10.0E+03 |
| 59 | 293 | 365 | 1130 |
| 60 | 117 | 124 | 2850 |
| 61 | 315 | 97.2 | >10.0E+03 |
| 62 | 140 | 773 | 3360 |
| 63 | 276 | 241 | 4090 |
| 64 | 58.9 | 61.5 | 1420 |
| 65 | 65.9 | 38.6 | 3550 |
| 66 | 373 | 202 | 2950 |
| 68 | 457 | 518 | 3470 |
| 69 | >10.0E+03 | >10.0E+03 | >10.0E+03 |
| 70 | 290 | 153 | 4580 |
| 71 | 400 | 320 | 4950 |
| 72 | 1420 | 5170 | >10.0E+03 |
| 73 | 351 | 214 | 5000 |
| 74 | 162 | 135 | 3230 |
| 75 | 212 | 161 | 3000 |
| 76 | 154 | 169 | 2370 |
| 77 | 462 | 673 | >8.08E+03 |
| 78 | 1440 | 2060 | >10.0E+03 |
| 80 | 276 | 213 | 4770 |
| 81 | 7400 | 4940 | >10.0E+03 |
| 82 | 895 | 231 | >10.0E+03 |
| 83 | 5950 | 1270 | >10.0E+03 |
| 84 | 475 | 339 | 879 |
| 85 | 124 | 527 | >8.83E+03 |
| 86 | 1040 | 765 | >10.0E+03 |
| 87 | 125 | 261 | 267 |
| 88 | 89.7 | 642 | >9.97E+03 |
| 89 | 1560 | 3900 | >5.82E+03 |
| 90 | 48.2 | 158 | 950 |
| 91 | 1200 | 859 | >10.0E+03 |
| 92 | 230 | 79.1 | 9060 |
| 93 | 1170 | 158 | >10.0E+03 |
| 94 | 1870 | 1750 | >10.0E+03 |
| 95 | 988 | 324 | 5780 |
| 96 | 1340 | 1150 | |
| 97 | 709 | 1160 | 7950 |
| 98 | 204 | 200 | 1830 |
| 99 | 99.6 | 229 | 1290 |
| 100 | >10.0E+03 | >10.0E+03 | >10.0E+03 |
| 101 | 1310 | 1480 | 2570 |
| 102 | 6900 | >10.0E+03 | >10.0E+03 |
| 104 | 5310 | 5280 | >10.0E+03 |
| 105 | >10.0E+03 | >10.0E+03 | >10.0E+03 |
| 107 | 1270 | 1220 | 2980 |
| 108 | 435 | 421 | >10.0E+03 |
| 109 | 60.5 | 54.5 | 636 |
| 110 | 43.4 | 40.6 | 1490 |
| 111 | 1790 | 771 | 4610 |
| 112 | >10.0E+03 | >10.0E+03 | 9870 |
| 121 | 321 | 345 | >10.0E+03 |
| 122 | 442 | 790 | 5260 |
| 123 | >10.0E+03 | 3800 | >10.0E+03 |
| 125 | 919 | 1780 | 4900 |
| 126 | 1780 | 1790 | 2030 |
| 127 | 3400 | 5950 | >10.0E+03 |
| 130 | >10.0E+03 | 5500 | 6430 |
| 131 | 1950 | 5600 | >10.0E+03 |
| 132 | 2470 | 8700 | >10.0E+03 |
| 133 | 7620 | >10.0E+03 | >10.0E+03 |
| 134 | >10.0E+03 | 1390 | >10.0E+03 |
| 135 | >10.0E+03 | >10.0E+03 | 5200 |

1. An EGFR protein covalently bound to a compound, wherein the compound is covalently bound to a lysine residue of the EGFR protein, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188.

2-14. (canceled)

15. An in vivo engineered EGFR protein comprising a non-naturally occurring reversible covalent modification at K745, the reversible covalent modification is generated from an in vivo nucleophilic reaction between an exogenous aromatic aldehyde and K745, wherein the exogenous aromatic aldehyde undergoes a nucleophilic addition with the nitrogen atom on K745 and forming an imine bond between the exogenous aromatic aldehyde and K745.

16-18. (canceled)

19. A method of inhibiting an EGFR protein, the method comprising contacting a lysine residue in EGFR with a reversible covalent inhibitor, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188.

20-24. (canceled)

25. A compound or pharmaceutically acceptable salt thereof that covalently binds to a lysine residue in EGFR, wherein the compound or a salt has a reversible covalent interaction with the lysine residue in EGFR, wherein the lysine residue is selected from K28, K29, K37, K80, K129, K133, K189, K209, K212, K226, K253, K261, K284, K293, K294, K325, K327, K328, K335, K346, K357, K360, K396, K399, K431, K454, K467, K478, K479, K487, K489, K500, K538, K593, K609, K642, K676, K708, K713, K714, K716, K728, K737, K739, K745, K754, K758, K806, K823, K846, K852, K860, K867, K875, K879, K913, K929, K949, K960, K970, K1061, K1099, K1160, K1179, K1182, and K1188.

26-29. (canceled)

30. A compound represented by the structure of Formula (I): 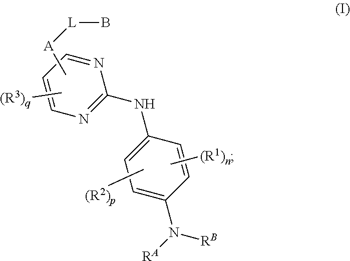
or a pharmaceutically acceptable salt thereof, wherein:
A is absent or -A¹-A²-;
A¹ is absent or selected from —O—, —S—, and —N(R⁴)—;
A² selected from a monocyclic 5- to 6-membered heteroarylene, bicyclic heteroarylene, bicyclic arylene, and phenylene, any of which are optionally substituted with one or more substituents selected from: halogen, —OR¹⁰, —N(R¹⁰)₂, —CN, —NO₂, and C₁₋₆ alkyl optionally substituted with one or more substituents independently selected from: halogen, —OR¹⁰, —N(R¹⁰)₂, —NO₂, and —CN;
L is absent or -L¹-L²-;
L¹ is absent or selected from —O—, —N(R⁵)—, and —S—;
L² is absent or selected from C₁₋₆ alkylene, C₂₋₆ alkenylene, and C₂₋₆ alkynylene, each of which is optionally substituted with one or more substituents independently selected from: halogen, —OR¹¹, —SR¹¹, —N(R¹¹)₂, —C(O)R¹¹, —C(O)OR¹¹, —OC(O)R¹¹, —OC(O)N(R¹¹)₂, —C(O)N(R¹¹)₂, —N(R¹¹)C(O)R¹¹, —N(R¹¹)C(O)OR¹¹, —N(R¹¹)C(O)N(R¹¹)₂, —N(R¹¹)S(O)₂(R¹¹), —S(O)R¹¹, —S(O)₂R¹¹, —S(O)₂N(R¹¹)₂, —NO₂, and —CN;
B is an amine-reactive lysine electrophile;
R¹ is an electrophile;
each R² is independently selected from: halogen, C₁₋₆ haloalkyl, C₁₋₆ hydroxyalkyl, C₁₋₆ alkoxy, C₁₋₆ haloalkoxy, —OR¹², —SR¹², —N(R¹²)₂, —C(O)OR¹², —OC(O)R¹² —C(O)N(R¹²)₂, —N(R¹²)C(O)R¹², —CN, and —NO₂;
each R³ is independently selected from:
halogen, —OR¹³, —SR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —NO₂, and —CN; and
C₁₋₆ alkyl, C₂₋₆ alkenyl, monocyclic 5- to 6-membered heteroaryl, bicyclic heteroaryl, bicyclic aryl, and phenyl, each of which is optionally substituted with one or more substituents independently selected from: halogen, —OR¹³, —SR¹³, —N(R¹³)₂, —C(O)R¹³, —C(O)OR¹³, —OC(O)R¹³, —OC(O)N(R¹³)₂, —C(O)N(R¹³)₂, —N(R¹³)C(O)R¹³, —N(R¹³)C(O)OR¹³, —N(R¹³)C(O)N(R¹³)₂, —N(R¹³)S(O)₂(R¹³), —S(O)R¹³, —S(O)₂R¹³, —S(O)₂N(R¹³)₂, —P(O)(R¹³)₂, —NO₂, C₁₋₆ alkyl, C₁₋₆ haloalkyl, and —CN;
R⁴ and R⁵ are each independently selected from hydrogen and C₁₋₆ alkyl optionally substituted with one or more substituents independently selected from: halogen, —OH, —NH₂, —NO₂, —CN, C₁₋₆ alkoxy, and C₁₋₆ haloalkoxy;
R^{A} and R^{B} are each independently selected from hydrogen; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; or
R^{A} and R^{B} come together to form a 4- to 10-membered heterocycle optionally substituted with one or more substituents independently selected from:
halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN;
C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, and —CN; and
C₃₋₁₀ carbocycle and 3- to 10-membered heterocycle, any of which is optionally substituted with one or more substituents independently selected from: halogen, —OR¹⁴, —SR¹⁴, —N(R¹⁴)₂, —C(O)R¹⁴, —C(O)OR¹⁴, —OC(O)R¹⁴, —OC(O)N(R¹⁴)₂, —C(O)N(R¹⁴)₂, —N(R¹⁴)C(O)R¹⁴, —N(R¹⁴)C(O)OR¹⁴, —N(R¹⁴)C(O)N(R¹⁴)₂, —N(R¹⁴)S(O)₂(R¹⁴), —S(O)R¹⁴, —S(O)₂R¹⁴, —S(O)₂N(R¹⁴)₂, —NO₂, —CN; and C₁₋₆ alkyl optionally substituted with one or more substituents independently selected from: halogen, —OR¹⁴, —SR¹⁴, —N(R¹⁴)₂, —C(O)R¹⁴, —C(O)OR¹⁴, —NO₂, and —CN;
R¹⁰, R¹¹, R¹², R¹³, and R¹⁴ are each independently selected at each occurrence from hydrogen, halogen, —OH, —NO₂, —CN, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ haloalkyl, C₁₋₆ hydroxyalkyl, C₁₋₆ alkoxy, and C₁₋₆ haloalkoxy;
n is selected from 0 and 1;
p is selected from 0, 1, 2, and 3; and
q is selected from 0, 1, and 2.

31-60. (canceled)

61. A compound represented by the structure of Formula (IA): 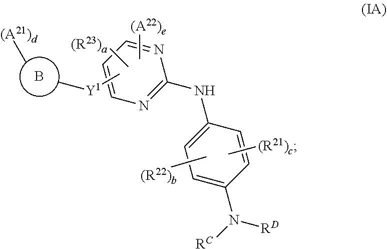
or a pharmaceutically acceptable salt thereof, wherein:
Y¹ is selected from a bond, —O—, —S—, and —N(R²⁴)—;
B is selected from a monocyclic 5- to 6-membered heteroaryl, bicyclic heteroaryl, bicyclic aryl, and phenyl, any of which is optionally substituted with one or more R²⁵;
A²¹ is represented by -L²¹-C²¹;
A²² is represented by -L²²-C²²;
L²¹ and L²² are each independently selected from a bond and -L^{A}-L^{B}-;
L^{A} is selected from a bond, —O—, —S—, and —N(R²⁶)—; and
L^{B} is selected from C₁₋₄ alkylene, C₂₋₄ alkenylene, and C₂₋₄ alkynylene, any of which is optionally substituted with one or more substituents independently selected from: halogen, —OR³¹, —SR³¹, —N(R³¹)₂, —C(O)R³¹, —C(O)OR³¹, —OC(O)R³¹, —OC(O)N(R³¹)₂, —C(O)N(R³¹)₂, —N(R³¹)C(O)R³¹, —N(R³¹)C(O)OR³¹, —N(R³¹)C(O)N(R³¹)₂, —N(R³¹)S(O)₂(R³¹), —S(O)R³¹, —S(O)₂R³¹, —S(O)₂N(R³¹)₂, ═O, ═S, ═NR³¹ —NO₂, and —CN;
C²¹ and C²² are each independently selected from phenyl and monocyclic 5- to 6-membered heteroaryl, any of which is optionally substituted with one or more substituents independently selected from: halogen, —OR³², —SR³², —N(R³²)₂, —C(O)H, —C(O)R³³, —C(O)OR³², —OC(O)R³², —OC(O)N(R³²)₂, —C(O)N(R³²)₂, —N(R³²)C(O)R³², —N(R³²)C(O)OR³², —N(R³²)C(O)N(R³²)₂, —N(R³²)S(O)₂(R³²), —S(O)R³², —S(O)₂R³², —S(O)₂N(R³²)₂, —NO₂, and —CN;
wherein at least one of C²¹, C²² and B is substituted by —C(O)H;
d is selected from 0 and 1;
e is selected from 0 and 1;
R²¹ is an electrophile;
R²² is independently selected at each occurrence from:
halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁴, —SR³⁴, —N(R³⁴)₂, —C(O)R³⁴, —C(O)OR³⁴, —OC(O)R³⁴, —OC(O)N(R³⁴)₂, —C(O)N(R³⁴)₂, —N(R³⁴)C(O)R³⁴, —N(R³⁴)C(O)OR³⁴, —N(R³⁴)C(O)N(R³⁴)₂, —N(R³⁴)S(O)₂(R³⁴), —S(O)R³⁴, —S(O)₂R³⁴, —S(O)₂N(R³⁴)₂, ═O, ═S, ═NR³⁴, —NO₂, and —CN;
R²³ is independently selected at each occurrence from:
halogen, —OR³, —SR³, —N(R³¹)₂, —C(O)R³⁵, —C(O)OR³⁵, —OC(O)R³¹, —OC(O)N(R³¹)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁵, —SR³⁵, —N(R³⁵)₂, —C(O)R³⁵, —C(O)OR³⁵, —OC(O)R³⁵, —OC(O)N(R³⁵)₂, —C(O)N(R³⁵)₂, —N(R³⁵)C(O)R³⁵, —N(R³⁵)C(O)OR³⁵, —N(R³⁵)C(O)N(R³⁵)₂, —N(R³⁵)S(O)₂(R³⁵), —S(O)R³⁵, —S(O)₂R³⁵, —S(O)₂N(R³⁵)₂, ═O, ═S, ═NR³⁵, —NO₂, and —CN;
R²⁴ is selected from hydrogen, C₁₋₄ alkyl, and C₁₄ haloalkyl;
R²⁵ is independently selected at each occurrence from:
halogen, —OR³⁶, —SR³⁶, —N(R³⁶)₂, —C(O)H, —C(O)R³⁷, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, —NO₂, and —CN; and
C₁₋₆ alkyl, C₂₋₆ alkenyl, and C₂₋₆ alkynyl, each of which is optionally substituted with one or more substituents independently selected from: halogen, —OR³⁶, —SR³⁶, —N(R³⁶)₂, —C(O)R³⁶, —C(O)OR³⁶, —OC(O)R³⁶, —OC(O)N(R³⁶)₂, —C(O)N(R³⁶)₂, —N(R³⁶)C(O)R³⁶, —N(R³⁶)C(O)OR³⁶, —N(R³⁶)C(O)N(R³⁶)₂, —N(R³⁶)S(O)₂(R³⁶), —S(O)R³⁶, —S(O)₂R³⁶, —S(O)₂N(R³⁶)₂, —P(O)(OR³⁶)₂, —P(O)R³⁶(OR³⁶), —P(O)(R³⁶)₂, ═O, ═S, ═NR³⁶, —NO₂, and —CN;
R²⁶ is selected at each occurrence from hydrogen, C₁₋₄ alkyl, and C₁4 haloalkyl;
a is selected from 0, 1, and 2.
b is selected from 0, 1, 2, 3, and 4;
c is selected from 0 and 1;
R^{C} and R^{D} are each independently selected from hydrogen and C₁₋₆ alkyl optionally substituted with one or more substituents independently selected from: halogen, —OR³⁸, —SR³⁸, —N(R³⁸)₂, —C(O)R³⁸, —C(O)OR³⁸, —OC(O)R³⁸, —OC(O)N(R³⁸)₂, —C(O)N(R³⁸)₂, —N(R³⁸)C(O)R³⁸, —N(R³⁸)C(O)OR³⁸, —N(R³⁸)C(O)N(R³⁸)₂, —N(R³⁸)S(O)₂(R³⁸), —S(O)R³⁸, —S(O)₂R³⁸, —S(O)₂N(R³⁸)₂, ═O, ═S, ═NR³⁸, —NO₂, and —CN; or
R^{C} and R^{D} come together to form a 4- to 10-membered heterocycle optionally substituted with one or more substituents independently selected from:
halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
C₁₋₆ alkyl optionally substituted with one or more substituents independently selected from: halogen, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN; and
C₃₋₁₀ carbocycle and 3- to 10-membered heterocycle, any of which is optionally substituted with one or more substituents independently selected from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R³¹, R³², R³⁴, R³⁵, R³⁶, R³⁸, and R³⁹ are each independently selected at each occurrence from: hydrogen, C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy; and
R³³ and R³⁷ are each independently selected at each occurrence from: C₁₋₆ alkyl, C₁₋₆ haloalkyl, C₁₋₆ hydroxyalkyl, C₁₋₆ alkoxy, C₁₋₆ haloalkoxy, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, ═O, —NO₂, —CN, C₁₋₆ alkyl, C₁₋₆ haloalkyl, C₁₋₆ hydroxyalkyl, C₁₋₆ alkoxy, and C₁₋₆ haloalkoxy.

62. (canceled)

63. The compound or salt of claim 61 or claim 0, wherein the compound is represented by Formula (II): 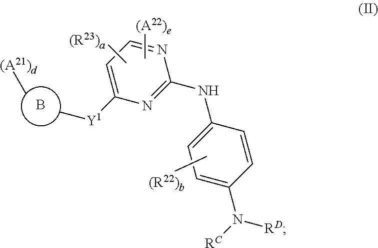
or a pharmaceutically acceptable salt thereof.

64-93. (canceled)

94. The compound or salt of claim 61, wherein the compound is a compound of Formula (IIIA): 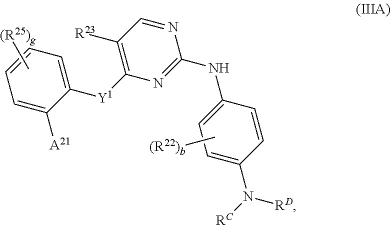
or a pharmaceutically acceptable salt thereof; wherein g is selected from 0, 1, 2, 3, and 4 or wherein the compound is a compound of Formula (IIIB): 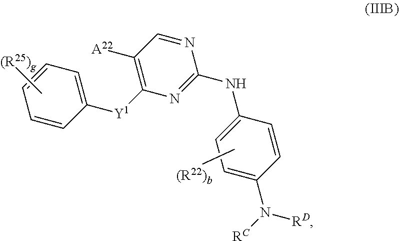
or a pharmaceutically acceptable salt thereof; wherein g is selected from 0, 1, 2, 3, and 4.

95-98. (canceled)

99. The compound or salt of claim 94, wherein the compound is a compound of Formula (IVA): 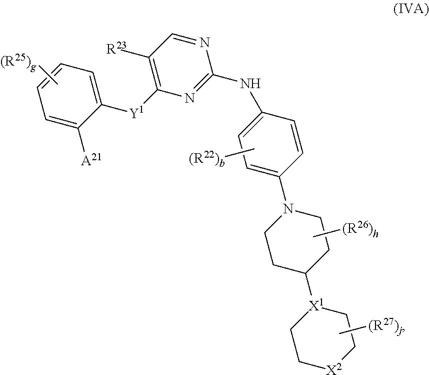
or a pharmaceutically acceptable salt thereof; wherein
X¹ is selected from C(R²⁸) and N;
X² is selected from C(R²⁸)₂ and N(R²⁹);
R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R²⁹ is selected from C₁₋₆ alkyl, C₁₋₆ haloalkyl, and C₁₋₆ hydroxyalkyl;
h is selected from 0, 1, 2, 3, 4, 5, 6, 7, and 8; and
j is selected from 0, 1, 2, 3, 4, 5, 6, 7, 8, and 9.

100. The compound or salt of claim 94, wherein the compound is a compound of Formula (IVb): 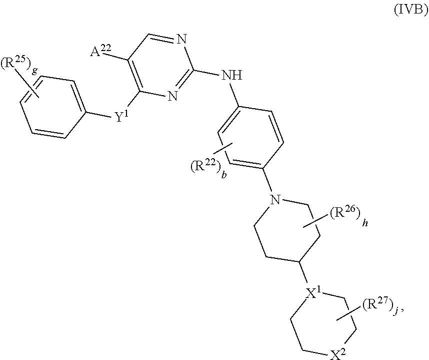
or a pharmaceutically acceptable salt thereof; wherein
X¹ is selected from C(R²⁸) and N;
X² is selected from C(R²⁸)₂ and N(R²⁹);
R²⁶, R²⁷, and R²⁸ are each independently selected at each occurrence from: halogen, C₁₋₆ alkyl, C₁₋₆ haloalkyl, C₁₋₆ hydroxyalkyl, —OR³⁹, —SR³⁹, —N(R³⁹)₂, —C(O)R³⁹, —C(O)OR³⁹, —OC(O)R³⁹, —OC(O)N(R³⁹)₂, —C(O)N(R³⁹)₂, —N(R³⁹)C(O)R³⁹, —N(R³⁹)C(O)OR³⁹, —N(R³⁹)C(O)N(R³⁹)₂, —N(R³⁹)S(O)₂(R³⁹), —S(O)R³⁹, —S(O)₂R³⁹, —S(O)₂N(R³⁹)₂, ═O, ═S, ═NR³⁹, —NO₂, and —CN;
R²⁹ is selected from C₁₋₆ alkyl, C₁₋₆ haloalkyl, and C₁₋₆ hydroxyalkyl;
h is selected from 0, 1, 2, 3, 4, 5, 6, 7, and 8; and
j is selected from 0, 1, 2, 3, 4, 5, 6, 7, 8, and 9.

101-106. (canceled)

107. The compound or salt of claim 61, wherein the compound is a compound of Formula (Va): 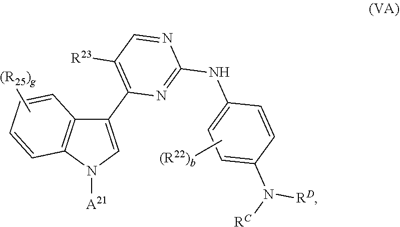
or a pharmaceutically acceptable salt thereof.

108. The compound or salt of claim 61, wherein the compound is a compound of Formula (Vb): 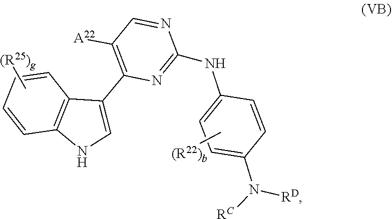
or a pharmaceutically acceptable salt thereof.

109-112. (canceled)

113. The compound of claim 61, wherein the compound is selected from: 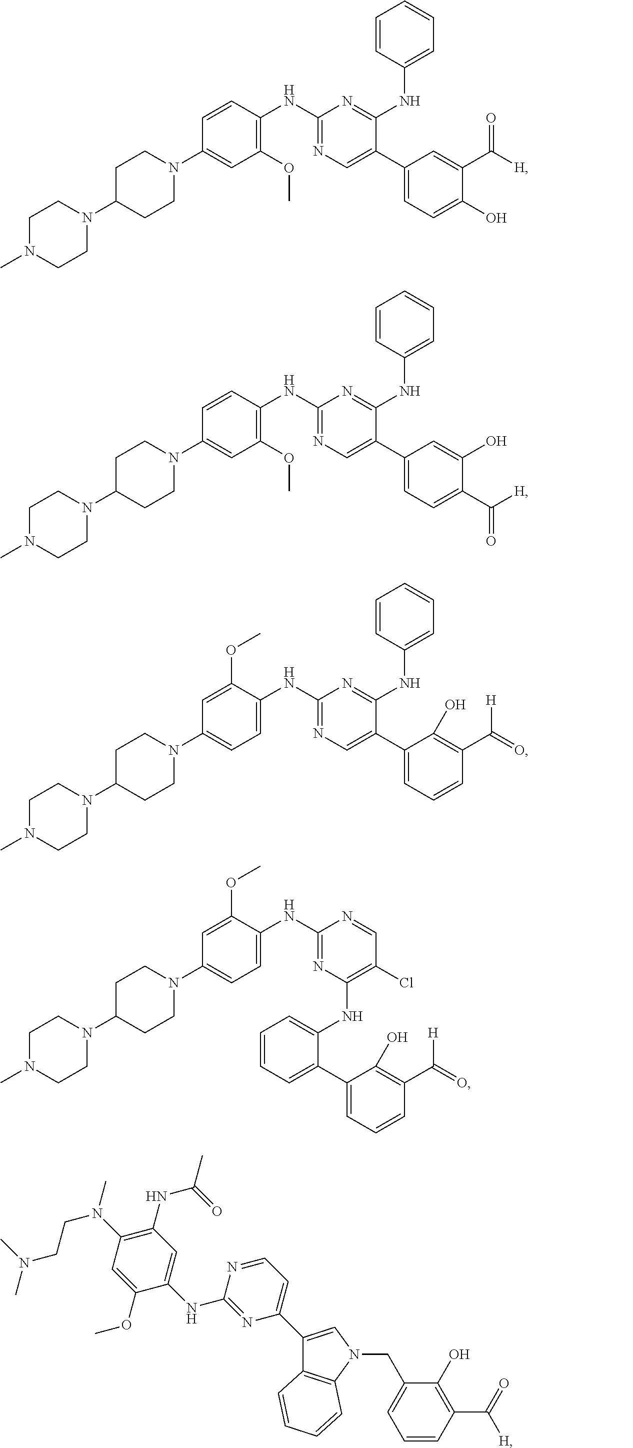 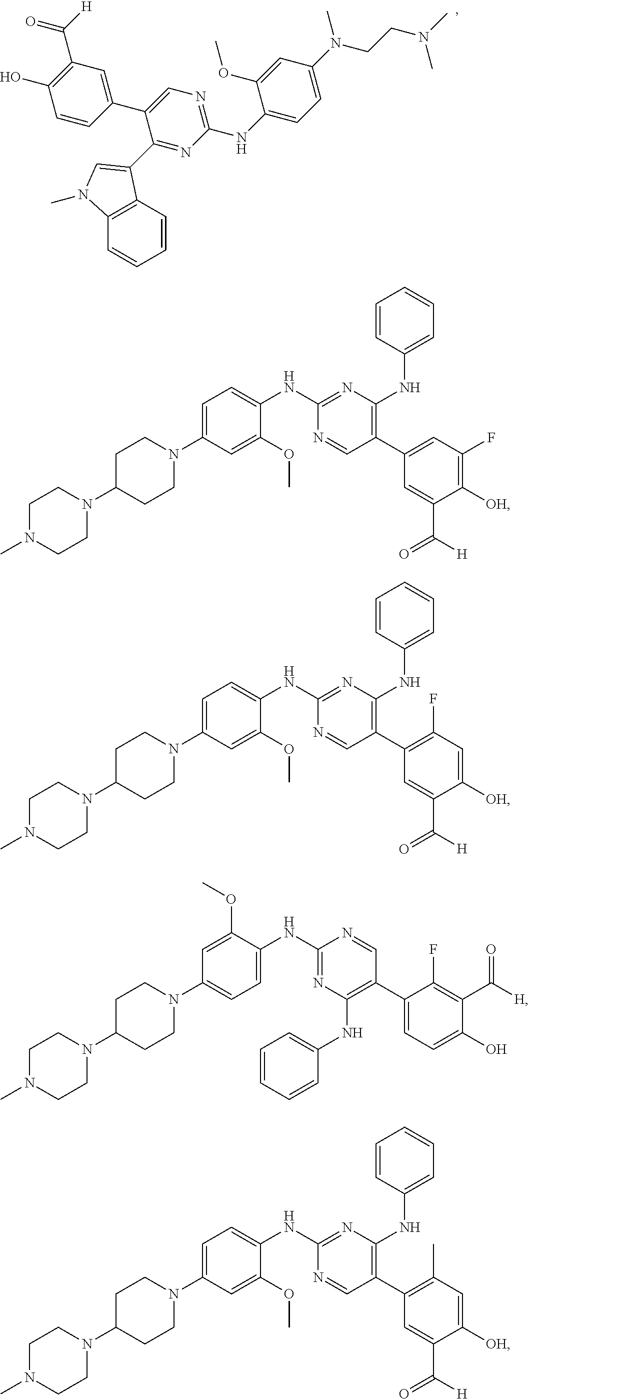 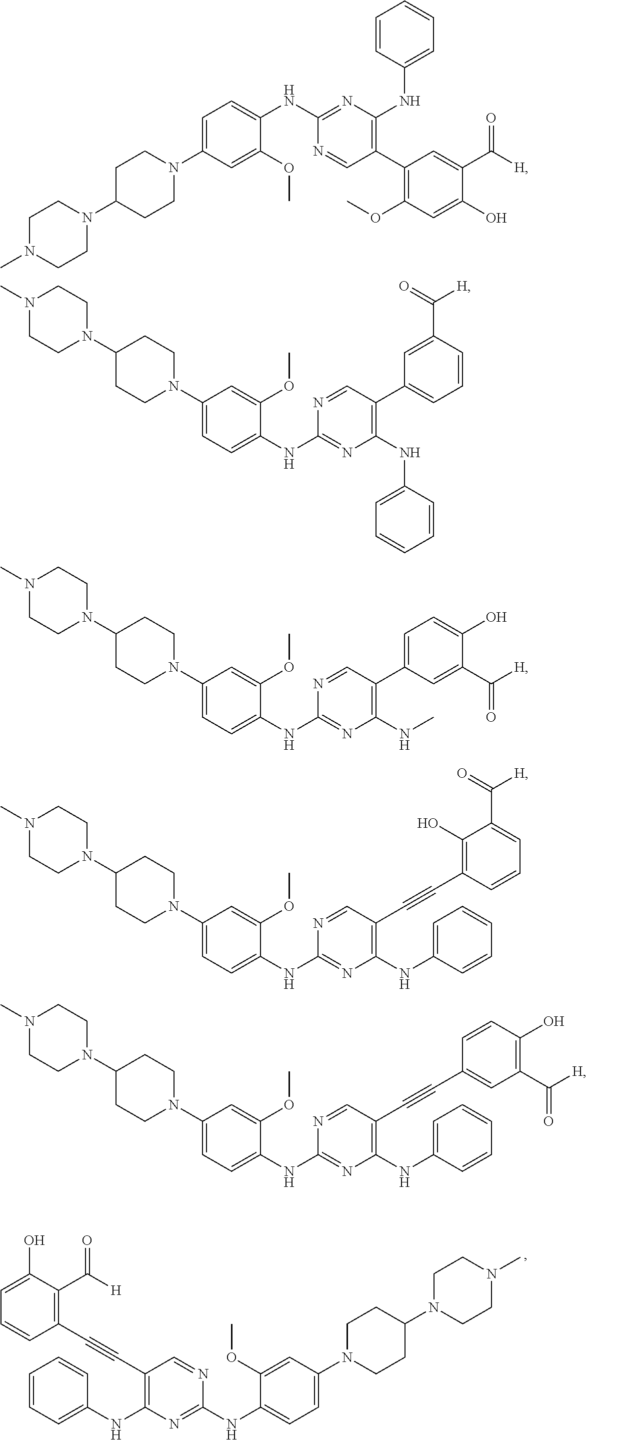 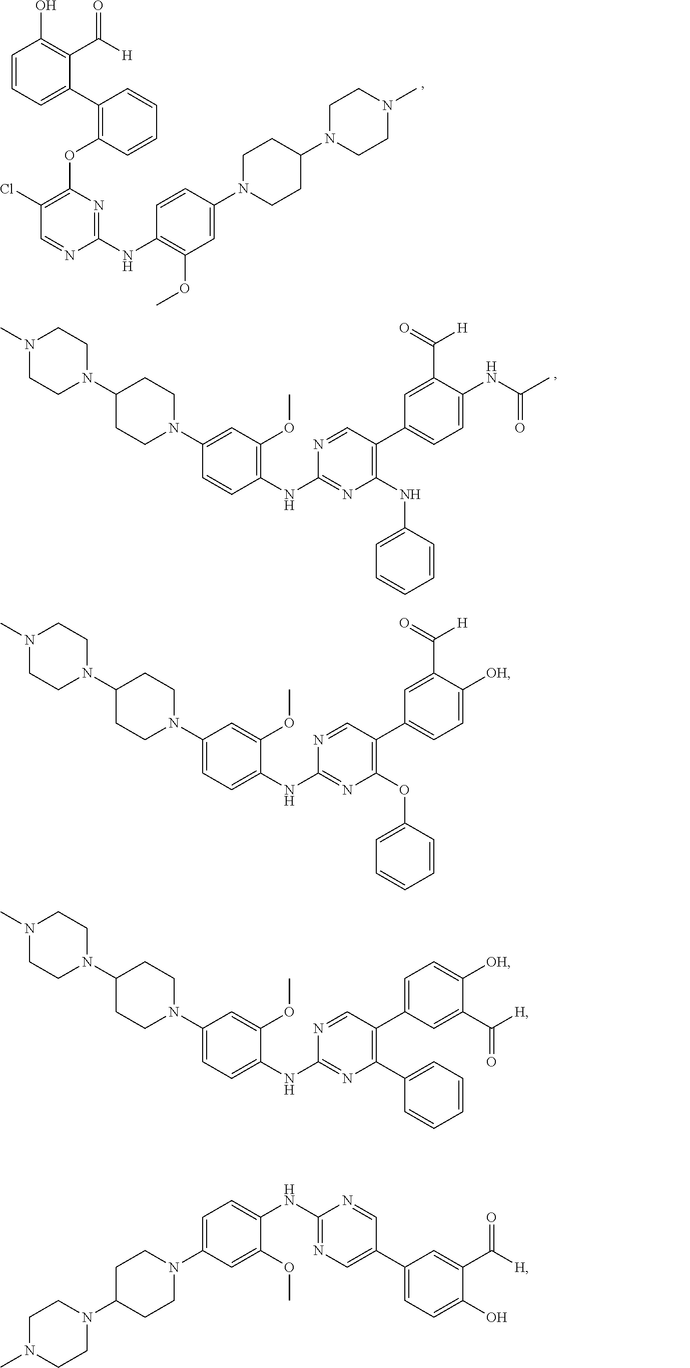 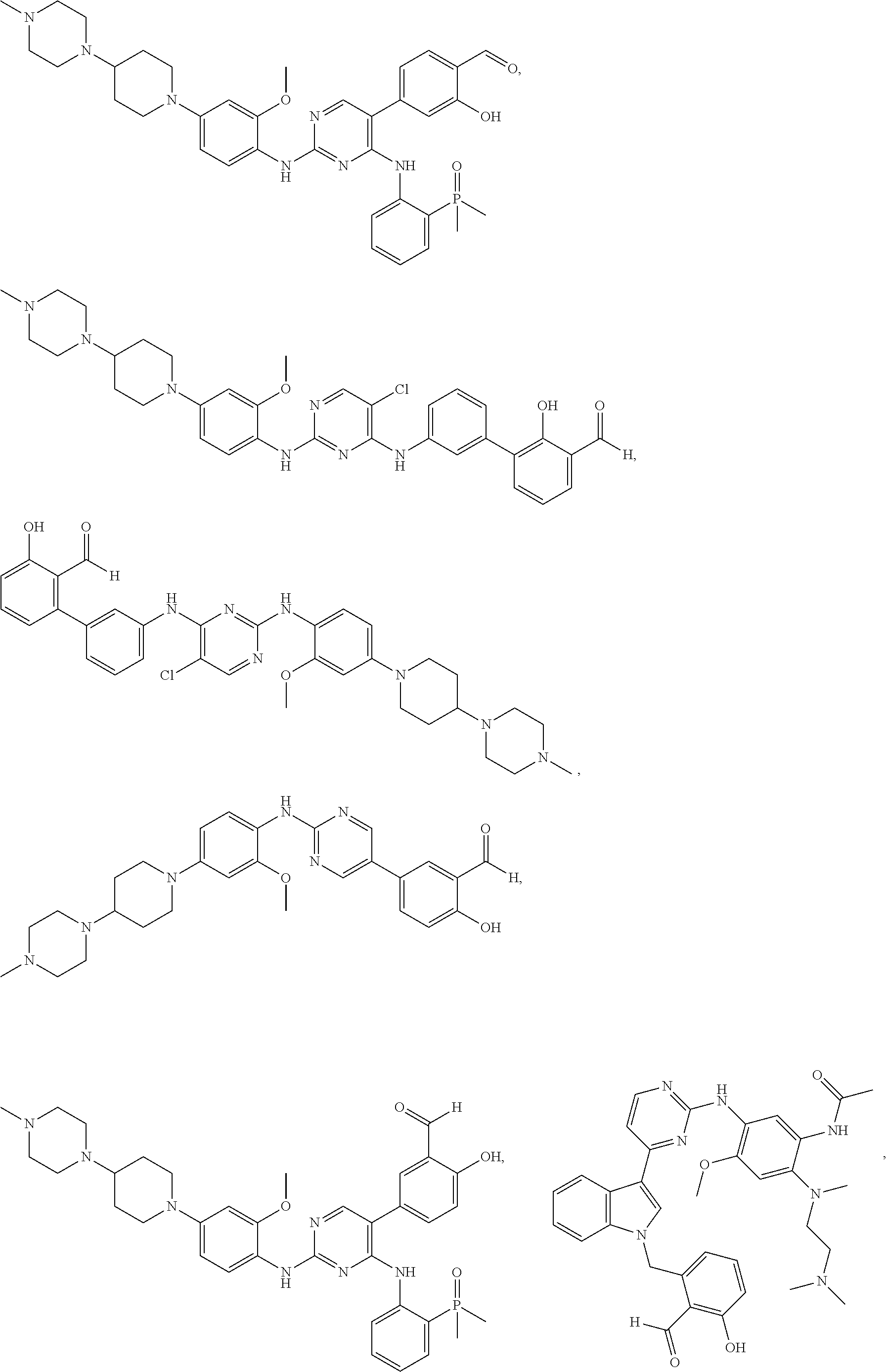 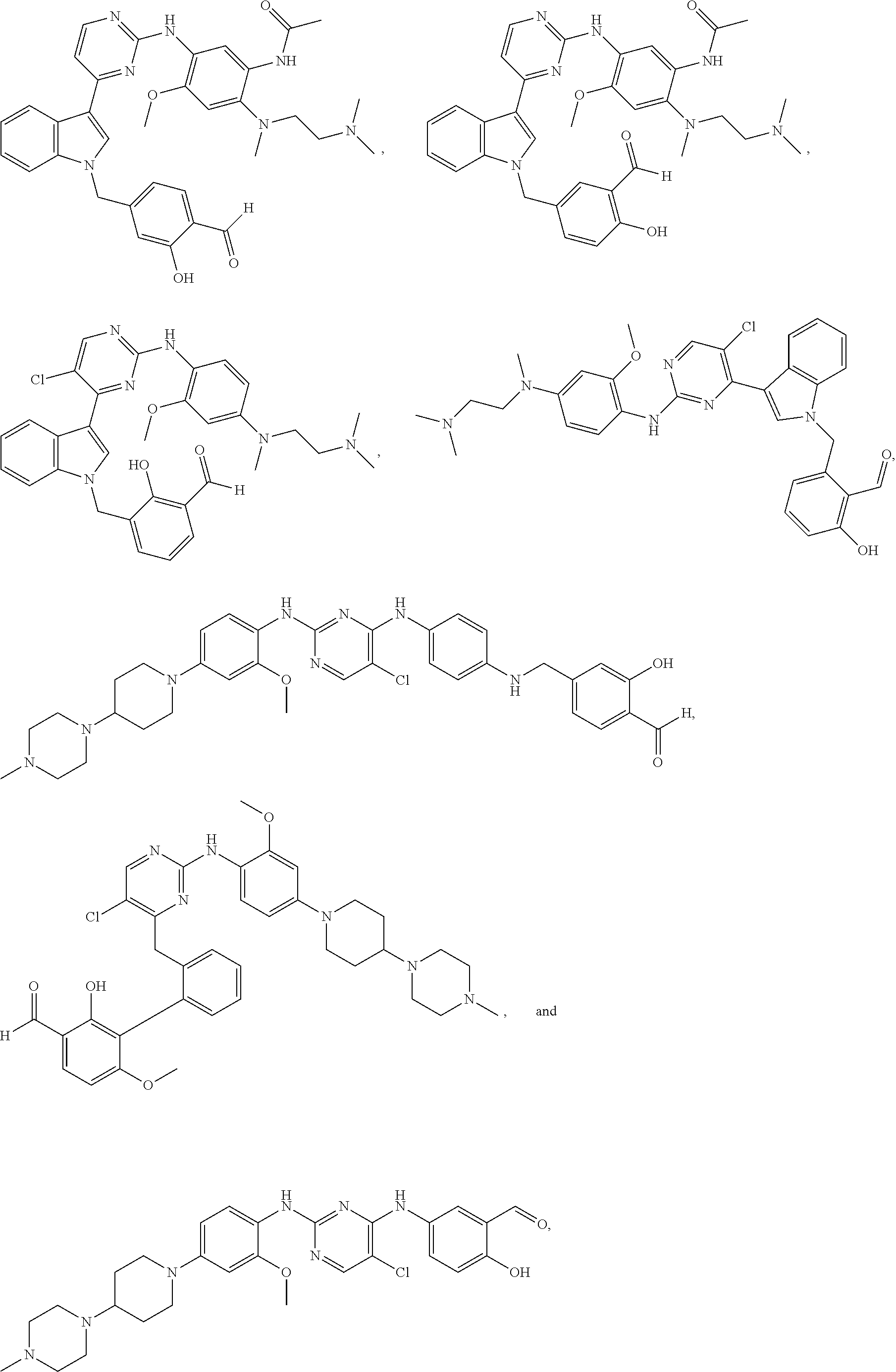
or a pharmaceutically acceptable salt thereof.

114. A compound represented by the structure of Formula (VI): 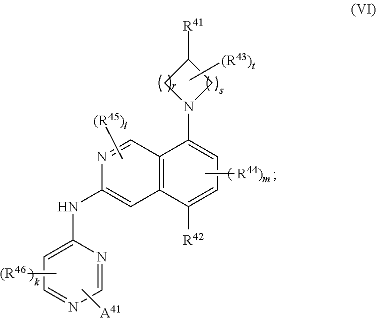
or a pharmaceutically acceptable salt thereof, wherein:
A⁴¹ is represented by -L⁴¹-C⁴¹;
L⁴¹ is selected from a bond and -L¹-L^{D}-;
L^{C} is selected from absent, —O—, —S—, and —N(R⁴⁷)—; and
L^{D} is selected from absent, C₁₋₄ alkylene, C₂₋₄ alkenylene, C₂₋₄ alkynylene, phenylene, and 5- to 6-membered heterocyclene, any of which is optionally substituted with one or more substituents independently selected from: halogen, —OR⁵¹, —SR⁵¹, —N(R⁵¹)₂, —C(O)R⁵¹, —C(O)OR⁵¹, —OC(O)R⁵¹, —OC(O)N(R⁵¹)₂, —C(O)N(R⁵¹)₂, —N(R⁵¹)C(O)R¹¹, —N(R¹¹)C(O)OR¹¹, —N(R¹¹)C(O)N(R¹¹)₂, —N(R⁵¹)S(O)₂(R¹¹), —S(O)R¹¹, —S(O)₂R¹¹, —S(O)₂N(R¹¹)₂, ═O, ═S, ═NR⁵¹ —NO₂, and —CN;
C⁴¹ is selected from phenyl and monocyclic 5- to 6-membered heteroaryl, any of which is substituted with —C(O)H and optionally substituted with one or more substituents independently selected from: halogen, —OR⁵², —SR⁵² —N(R⁵²)₂, —C(O)R⁵³, —C(O)OR⁵², —OC(O)R⁵², —OC(O)N(R⁵²)₂, —C(O)N(R⁵²)₂, —N(R⁵²)C(O)R⁵², —N(R⁵²)C(O)OR⁵², —N(R⁵²)C(O)N(R⁵²)₂, —N(R⁵²)S(O)₂(R⁵²), —S(O)R⁵², —S(O)₂R⁵², —S(O)₂N(R⁵²)₂, —NO₂, and —CN;
R⁴¹ is selected from C₁₋₄ alkyl optionally substituted with one or more substituent independently selected at each occurrence from: halogen, —OR⁵⁴, —SR⁵⁴, —N(R⁵⁴)₂, —C(O)R⁵⁴, —C(O)OR⁵⁴, —OC(O)R⁵⁴, —OC(O)N(R⁵⁴)₂, —C(O)N(R⁵⁴)₂, —N(R⁵⁴)C(O)R⁵⁴, —N(R⁵⁴)C(O)OR⁵⁴, —N(R⁵⁴)C(O)N(R⁵⁴)₂, —N(R⁵⁴)S(O)₂(R⁵⁴), —S(O)R⁵⁴, —S(O)₂R⁵⁴, —S(O)₂N(R⁵⁴)₂, ═O, ═S, ═NR⁵⁴, —NO₂, and —CN;
R⁴² is selected from C₁₋₆ alkyl optionally substituted with one or more substituent independently selected at each occurrence from: halogen, —OR⁵⁵ —SR⁵⁵ —N(R⁵⁵)₂, —C(O)R⁵⁵, —C(O)OR⁵⁵, —OC(O)R⁵⁵, —OC(O)N(R⁵⁵)₂, —C(O)N(R⁵⁵)₂, —N(R⁵⁵)C(O)R⁵⁵, —N(R⁵⁵)C(O)OR⁵⁵, —N(R⁵⁵)C(O)N(R⁵⁵)₂, —N(R⁵⁵)S(O)₂(R⁵⁵), —S(O)R⁵⁵, —S(O)₂R⁵⁵, —S(O)₂N(R⁵⁵)₂, ═O, ═S, ═NR⁵⁵, —NO₂, and —CN;
R⁴³ is selected from halogen, C₁₋₆ alkyl, C₁₋₆ haloalkyl, —OR⁵⁶, —SR⁵⁶, —N(R⁵⁶)₂, —C(O)R⁵⁶, —C(O)OR⁵⁶, —OC(O)R⁵⁶, —OC(O)N(R⁵⁶)₂, —C(O)N(R⁵⁶)₂, —N(R⁵⁶)C(O)R⁵⁶, —N(R⁵⁶)C(O)OR⁵⁶, —N(R⁵⁶)C(O)N(R⁵⁶)₂, —N(R⁵⁶)S(O)₂(R⁵⁶), —S(O)R⁵⁶, —S(O)₂R⁵⁶, —S(O)₂N(R⁵⁶)₂, —NO₂, and —CN; or
two R⁴³ attached to the same carbon atom together form ═O, ═S, and ═NR⁵⁶;
R⁴⁴, R⁴⁵, and R⁴⁶ are each independently selected at each occurrence from: halogen, C₁₋₆ alkyl, C₁₋₆ haloalkyl, —OR⁵⁷, —SR⁵⁷, —N(R⁵⁷)₂, —C(O)R⁵⁷, —C(O)OR⁵⁷, —OC(O)R⁵⁷, —OC(O)N(R⁵⁷)₂, —C(O)N(R⁵⁷)₂, —N(R⁵⁷)C(O)R⁵⁷, —N(R⁵⁷)C(O)OR⁵⁷, —N(R⁵⁷)C(O)N(R⁵⁷)₂, —N(R⁵⁷)S(O)₂(R⁵⁷), —S(O)R⁵⁷, —S(O)₂R⁵⁷, —S(O)₂N(R⁵⁷)₂, —NO₂, and —CN;
R⁴⁷ is selected from hydrogen, C₁₋₆ alkyl, and C₁₋₆ haloalkyl;
R⁵¹, R⁵², R⁵⁴, R⁵⁵, R⁵⁶, and R⁵⁷ are each independently selected at each occurrence from: hydrogen, C₁₋₆ alkyl, C₁₋₆ haloalkyl, C₁₋₆ hydroxyalkyl, C₁₋₆ alkoxy, and C₁₋₆ haloalkoxy; and
R⁵³ is selected at each occurrence from: C₁₋₆ alkyl, C₁₋₆ haloalkyl, C₁₋₆ hydroxyalkyl, C₁₋₆ alkoxy, and C₁₋₆ haloalkoxy;
r and s are each independently selected from 1 and 2;
t is selected from 0, 1, 2, 3, and 4; and
k, l, and m are each independently selected from 0, 1, and 2.

115-137. (canceled)

138. The compound or salt of claim 114 selected from: 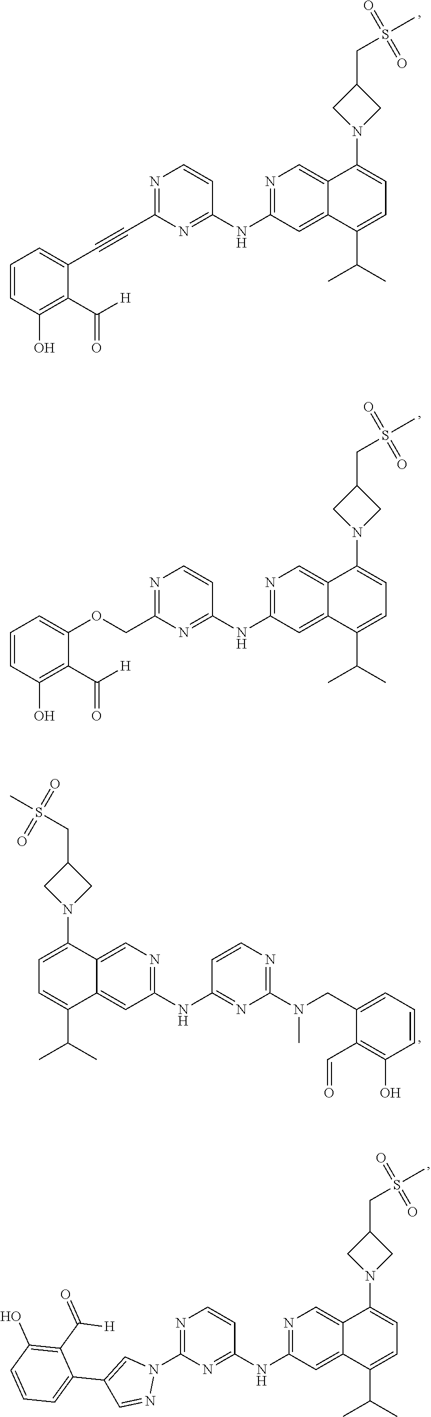 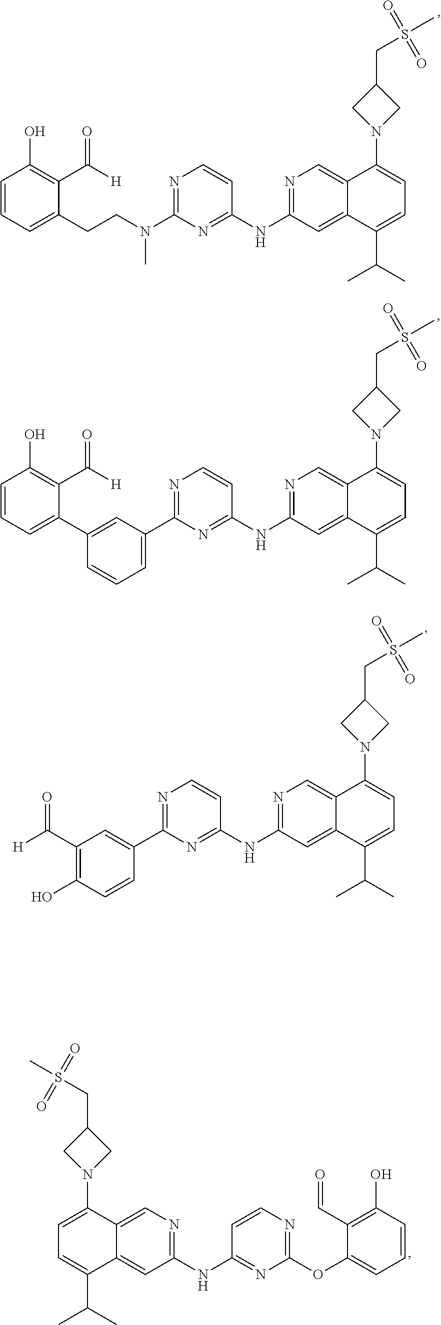 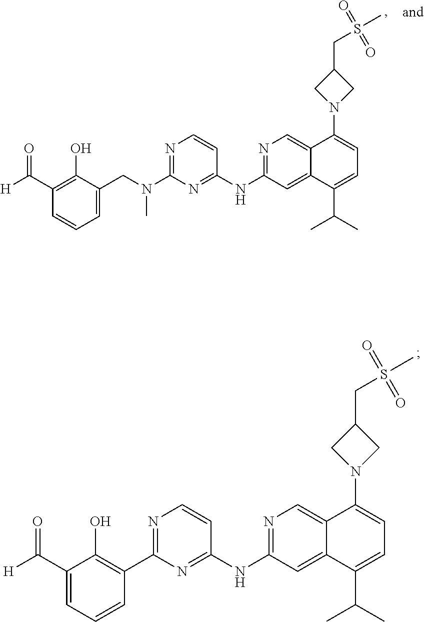 [SMILES: CC(C)c1ccc(N2CC(CS(C)(=O)=O)C2)c2cnc(Nc3ccnc(N(C)Cc4cccc(C=O)c4O)n3)cc12 ; CC(C)c1ccc(N2CC(CS(C)(=O)=O)C2)c2cnc(Nc3ccnc(-c4cccc(C=O)c4O)n3)cc12]
or a pharmaceutically acceptable salt thereof.

139. A compound represented by the structure of Formula (VIIA) or (VIIB): 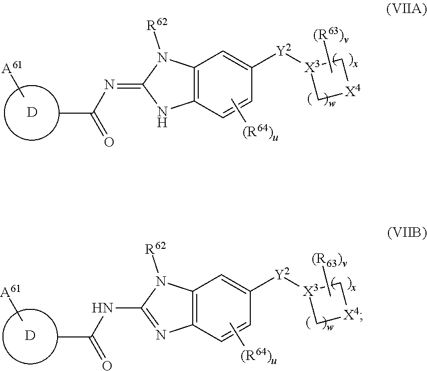
or a pharmaceutically acceptable salt thereof, wherein:
D is a phenyl or a 5- to 10-membered heteroaryl optionally substituted with one or more substituents independently selected from: halogen, C₁₋₆ alkyl, C₁₋₆ haloalkyl, —OR⁷⁰, —SR⁷⁰, —N(R⁷⁰)₂, —C(O)R⁷⁰, —C(O)OR⁷⁰, —OC(O)R⁷⁰, —OC(O)N(R⁷⁰)₂, —C(O)N(R⁷⁰)₂, —N(R⁷⁰)C(O)R⁷⁰, —N(R⁷⁰)C(O)OR⁷⁰, —N(R⁷⁰)C(O)N(R⁷⁰)₂, —N(R⁷⁰)S(O)₂(R⁷⁰), —S(O)R⁷⁰, —S(O)₂R⁷⁰, —S(O)₂N(R⁷⁰)₂, —NO₂, and —CN;
A⁶¹ is represented by *-L⁶¹-C⁶¹ wherein * represents the connection to D;
L⁶¹ is selected from a bond and -L^{E}-L^{F}-;
L^{E} is selected from absent, —O—, —S—, and —N(R⁶⁶)—; and
L^{F} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷¹, —SR⁷¹, —N(R⁷¹)₂, —C(O)R⁷¹, —C(O)OR⁷¹, —OC(O)R⁷¹, —OC(O)N(R⁷¹)₂, —C(O)N(R⁷¹)₂, —N(R⁷¹)C(O)R⁷¹, —N(R⁷¹)C(O)OR⁷¹, —N(R⁷¹)C(O)N(R⁷¹)₂, —N(R⁷¹)S(O)₂(R⁷¹), —S(O)R⁷¹, —S(O)₂R⁷¹, —S(O)₂N(R⁷¹)₂, ═O, ═S, ═NR⁷¹, —NO₂, and —CN;
C⁶¹ is selected from phenyl and monocyclic 5- to 6-membered heteroaryl, any of which is substituted with —C(O)H and optionally substituted with one or more substituents independently selected from: halogen, —OR⁷², —SR⁷², —N(R⁷²)₂, —C(O)R⁷², —C(O)OR⁷², —OC(O)R⁷², —OC(O)N(R⁷²)₂, —C(O)N(R⁷²)₂, —N(R⁷²)C(O)R⁷², —N(R⁷²)C(O)OR⁷², —N(R⁷²)C(O)N(R⁷²)₂, —N(R⁷²)S(O)₂(R⁷²), —S(O)R⁷², —S(O)₂R⁷², —S(O)₂N(R⁷²)₂, —NO₂, and —CN;
Y² is selected from a bond and C₁₋₄ alkylene optionally substituted with one or more substituents selected from halogen, —OH, —O—C₁₋₄ alkyl, ═O, and ═S;
X³ is CR⁶⁵ or N;
X⁴ is O, C(R⁶⁵)(R⁶¹), or N(R⁶¹);
R⁶¹ is independently selected from:
hydrogen;
C₁₋₆ alkyl optionally substituted with one or more substituents selected from halogen, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN; and
5- to 6-membered heterocycle optionally substituted with one or more substituents selected from halogen, C₁₋₄ alkyl, C₁₄ haloalkyl, —OR⁷³, —SR⁷³, —N(R⁷³)₂, —C(O)R⁷³, —C(O)OR⁷³, —OC(O)R⁷³, —OC(O)N(R⁷³)₂, —C(O)N(R⁷³)₂, —N(R⁷³)C(O)R⁷³, —N(R⁷³)C(O)OR⁷³, —N(R⁷³)C(O)N(R⁷³)₂, —N(R⁷³)S(O)₂(R⁷³), —S(O)R⁷³, —S(O)₂R⁷³, —S(O)₂N(R⁷³)₂, —NO₂, and —CN;
R⁶² is selected from C₁₋₆ alkyl optionally substituted with one or more substituent independently selected at each occurrence from: halogen, —OR⁷⁴, —SR⁷⁴, —N(R⁷⁴)₂, —C(O)R⁷⁴, —C(O)OR⁷⁴, —OC(O)R⁷⁴, —OC(O)N(R⁷⁴)₂, —C(O)N(R⁷⁴)₂, —N(R⁷⁴)C(O)R⁷⁴, —N(R⁷⁴)C(O)OR⁷⁴, —N(R⁷⁴)C(O)N(R⁷⁴)₂, —N(R⁷⁴)S(O)₂(R⁷⁴), —S(O)R⁷⁴, —S(O)₂R⁷⁴, —S(O)₂N(R⁷⁴)₂, ═O, ═S, ═NR⁷⁴, —NO₂, and —CN;
R⁶³ and R⁶⁴ are each independently selected at each occurrence from halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, —N(R⁷⁵)S(O)₂(R⁷⁵), —S(O)R⁷⁵, —S(O)₂R⁷⁵, —S(O)₂N(R⁷⁵)₂, —NO₂, and —CN;
R⁶⁵ is selected from hydrogen, halogen, C₁₋₆ alkyl, C₁₋₆ haloalkyl, —OR⁷⁵, —SR⁷⁵, —N(R⁷⁵)₂, —C(O)R⁷⁵, —C(O)OR⁷⁵, —OC(O)R⁷⁵, —C(O)N(R⁷⁵)₂, —N(R⁷⁵)S(O)₂(R⁷⁵), —S(O)R⁷⁵, —S(O)₂R⁷⁵, —S(O)₂N(R⁷⁵)₂, —NO₂, and —CN;
R⁶⁶ is selected from hydrogen, C₁₋₆ alkyl, and C₁₋₆ haloalkyl;
R⁷⁰, R⁷¹, R⁷², R⁷³, R⁷⁴, and R⁷⁵ are each independently selected at each occurrence from:
hydrogen, C₁₋₆ alkyl, C₁₋₆ haloalkyl, C₁₋₆ hydroxyalkyl, C₃₋₆ carbocycle, and 3- to 6-membered heterocycle;
u is selected from 0, 1, 2, and 3.
v is selected from 0, 1, 2, 3, and 4; and
w and x are each independently selected from 1 and 2.

140-167. (canceled)

168. The compound or salt of claim 139 selected from: 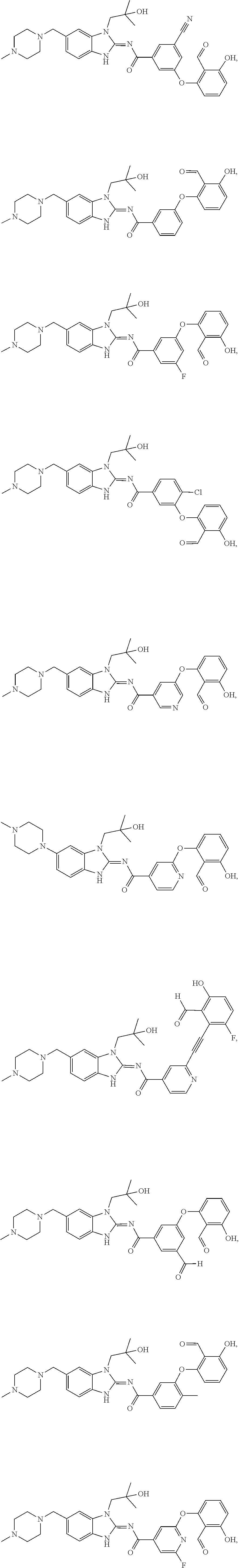 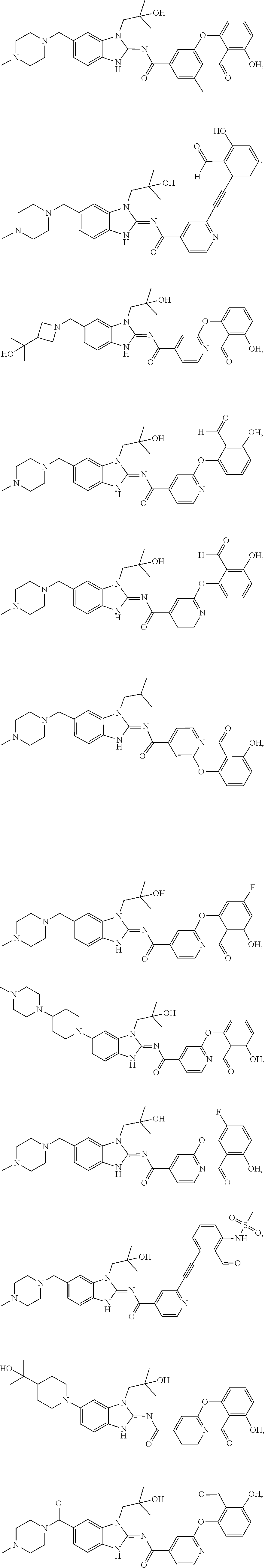 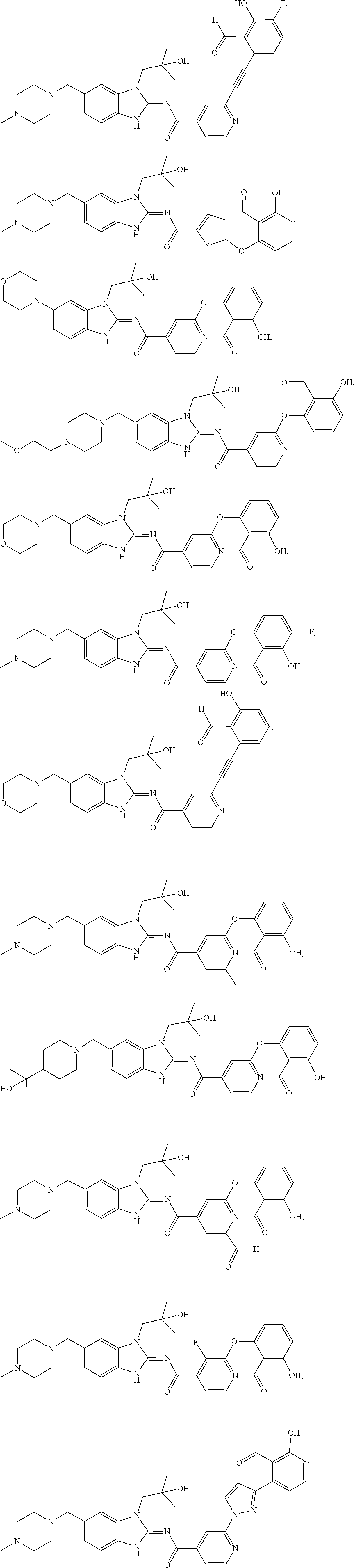 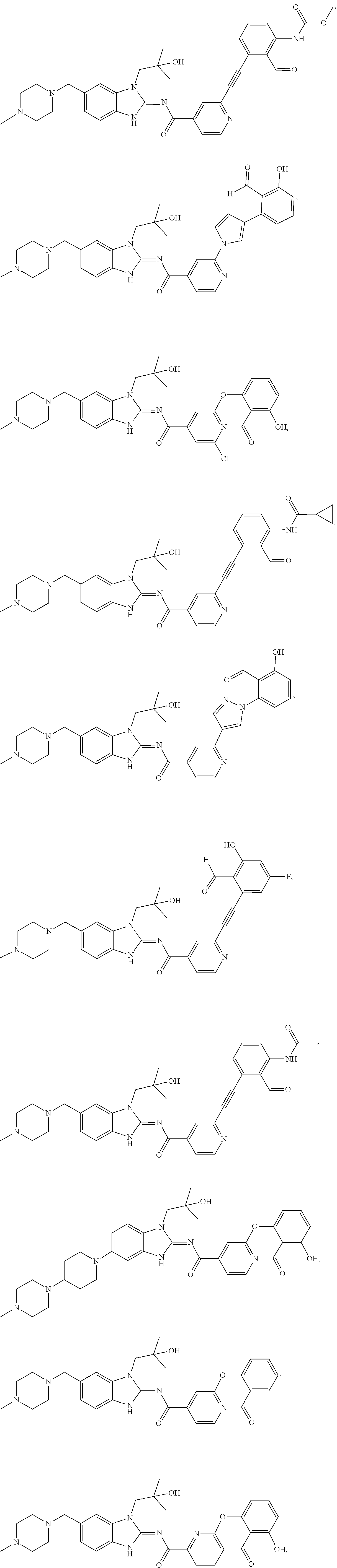 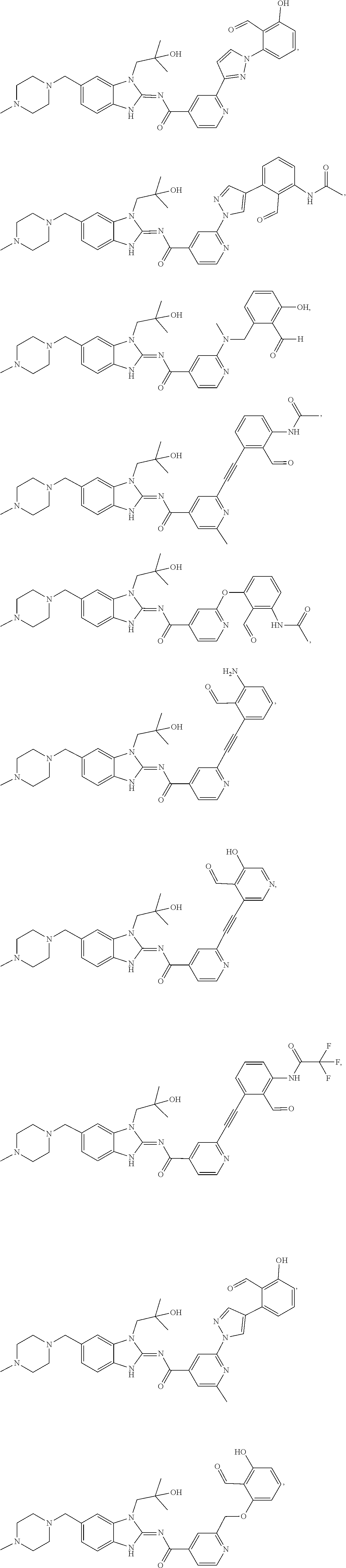 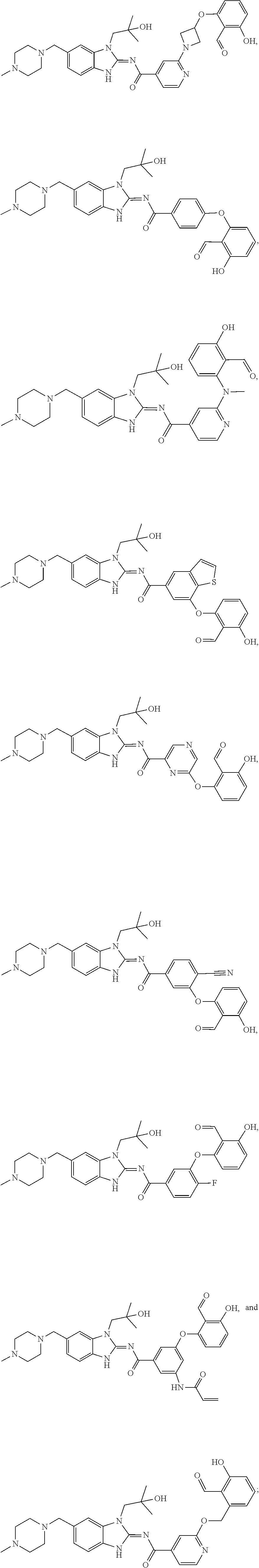
or a pharmaceutically acceptable salt thereof.

169. A compound represented by the structure of Formula (VIIIA) or (VIIIB): 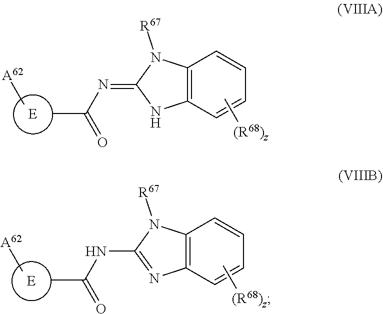
or a pharmaceutically acceptable salt thereof, wherein:
E is phenyl or 5- to 10-membered heteroaryl optionally substituted with one or more substituents independently selected from: halogen, C₁₋₆ alkyl, C₁₋₆ haloalkyl, —OR⁷⁶, —SR⁷⁶, —N(R⁷⁶)₂, —C(O)R⁷⁶, —C(O)OR⁷⁶, —OC(O)R⁷⁶, —OC(O)N(R⁷⁶)₂, —C(O)N(R⁷⁶)₂, —N(R⁷⁶)C(O)R⁷⁶, —N(R⁷⁶)C(O)OR⁷⁶, —N(R⁷⁶)C(O)N(R⁷⁶)₂, —N(R⁷⁶)S(O)₂(R⁷⁶), —S(O)R⁷⁶, —S(O)₂R⁷⁶, —S(O)₂N(R⁷⁶)₂, —NO₂, and —CN;
A⁶² is represented by *-L⁶²-C⁶², wherein * represents the connection to E;
L⁶² is selected from a bond and -L^{G}-L^{H}-;
L^{G} is selected from absent, —O—, —S—, and —N(R⁶⁹)—; and
L^{H} is selected from absent, C₁₋₄ alkylene, C₂₋₄ alkenylene, C₂₋₄ alkynylene, phenylene, and 4- to 6-membered heterocyclene, any of which is optionally substituted with one or more substituents independently selected from: halogen, —OR⁷⁷, —SR⁷⁷, —N(R⁷⁷)₂, —C(O)R⁷⁷, —C(O)OR⁷⁷, —OC(O)R⁷⁷, —OC(O)N(R⁷⁷)₂, —C(O)N(R⁷⁷)₂, —N(R⁷⁷)C(O)R⁷⁷, —N(R⁷⁷)C(O)OR⁷⁷, —N(R⁷⁷)C(O)N(R⁷⁷)₂, —N(R⁷⁷)S(O)₂(R⁷⁷), —S(O)R⁷⁷, —S(O)₂R⁷⁷, —S(O)₂N(R⁷⁷)₂, ═O, ═S, ═NR⁷⁷, —NO₂, and —CN;
C⁶² is selected from phenyl and monocyclic 5- to 6-membered heteroaryl, any of which is substituted with —C(O)H and optionally substituted with one or more substituents independently selected from: halogen, —OR⁷⁸, —SR⁷⁸, —N(R⁷⁸)₂, —C(O)R⁷⁸, —C(O)OR⁷⁸, —OC(O)R⁷⁸, —OC(O)N(R⁷⁸)₂, —C(O)N(R⁷⁸)₂, —N(R⁷⁸)C(O)R⁷⁸, —N(R⁷⁸)C(O)OR⁷⁸, —N(R⁷⁸)C(O)N(R⁷⁸)₂, —N(R⁷⁸)S(O)₂(R⁷⁸), —S(O)R⁷⁸, —S(O)₂R⁷⁸, —S(O)₂N(R⁷⁸)₂, —NO₂, and —CN;
R⁶⁷ is independently selected from:
C₁₋₆ alkyl, C₁₋₆ alkenyl, and C₁₋₆ alkynyl, each of which is optionally substituted with one or more substituents selected from:
halogen, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN; and
C₃₋₆ carbocycle, and 5- to 6-membered heterocycle, wherein the C₃O₆ carbocycle and 5- to 6-membered heterocycle are each optionally substituted with one or more substituents selected from halogen, C₁₄ alkyl, C₁₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN;
C₃₋₈ carbocycle and 3- to 8-membered heterocycle, each of which is optionally substituted with one or more substituents selected from halogen, C₁₋₄ alkyl, C₁₄ haloalkyl, —OR⁷⁹, —SR⁷⁹, —N(R⁷⁹)₂, —C(O)R⁷⁹, —C(O)OR⁷⁹, —OC(O)R⁷⁹, —OC(O)N(R⁷⁹)₂, —C(O)N(R⁷⁹)₂, —N(R⁷⁹)C(O)R⁷⁹, —N(R⁷⁹)C(O)OR⁷⁹, —N(R⁷⁹)C(O)N(R⁷⁹)₂, —N(R⁷⁹)S(O)₂(R⁷⁹), —S(O)R⁷⁹, —S(O)₂R⁷⁹, —S(O)₂N(R⁷⁹)₂, ═O, ═S, —NO₂, and —CN;
R⁶⁸ are each independently selected at each occurrence from halogen, C₁₋₆ alkyl, C₁₋₆ haloalkyl, —OR⁸⁰, —SR⁸⁰, —N(R⁸⁰)₂, —C(O)R⁸⁰, —C(O)OR⁸⁰, —OC(O)R⁸⁰, —C(O)N(R⁸⁰)₂, —N(R⁸⁰)S(O)₂(R⁸⁰), —S(O)R⁸⁰, —S(O)₂R⁸⁰, —S(O)₂N(R⁸⁰)₂, —NO₂, and —CN;
R⁶⁹ is selected from hydrogen, C₁₋₆ alkyl, and C₁₋₆ haloalkyl;
R⁷⁹ is selected at each occurrence from:
hydrogen;
C₁₋₆ alkyl, C₁₋₆ alkenyl, and C₁₋₆ alkynyl each of which is optionally substituted with one or more substituents selected from halogen, —OR⁸¹, —SR⁸¹, —N(R⁸¹)₂, —C(O)R⁸¹, —C(O)OR⁸¹, —OC(O)R⁸¹, —C(O)N(R⁸¹)₂, —N(R⁸¹)C(O)R⁸¹, —N(R⁸¹)S(O)₂(R⁸¹), —S(O)R⁸¹, —S(O)₂R⁸¹, —S(O)₂N(R⁸¹)₂, ═O, ═S, —NO₂, and —CN;
C₃₋₆ carbocycle and 5- to 6-membered heterocycle, each of which is optionally substituted with one or more substituents selected from halogen, C₁₋₄ alkyl, C₁₋₄ haloalkyl, —OR⁸¹, —SR⁸¹, —N(R⁸¹)₂, —C(O)R⁸¹, —C(O)OR⁸¹ , —OC(O)R⁸¹ , —C(O)N(R⁸¹)₂, —N(R⁸¹)C(O)R⁸¹, —N(R⁸¹)S(O)₂(R⁸¹), —S(O)R⁸¹, —S(O)₂R⁸¹, —S(O)₂N(R⁸¹)₂, ═O, ═S, —NO₂, and —CN;
R⁷⁶, R⁷⁷, R⁷⁸, R⁸⁰, and R⁸¹ are each independently selected at each occurrence from hydrogen, C₁₋₆ alkyl, and C₁₋₆ haloalkyl; and
z is selected from 0, 1, 2, and 3.

170-173. (canceled)

174. The compound or salt of claim 169, wherein the compound is represented by the structure of Formula (IXA) or (IXB): 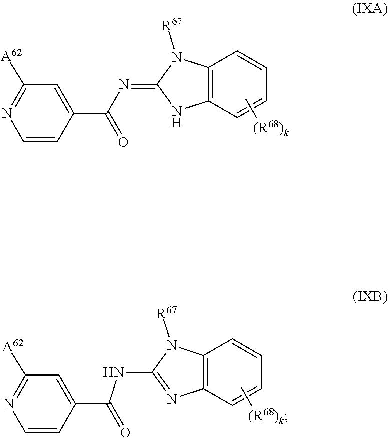
or a pharmaceutically acceptable salt thereof.

175-196. (canceled)

197. The compound or salt of claim 169 selected from: 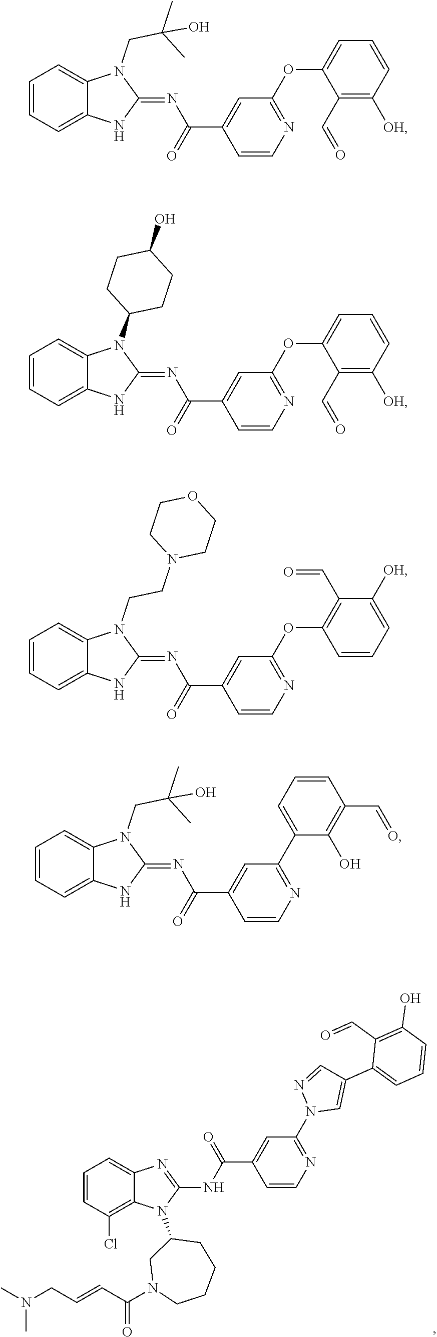 [SMILES: CC(C)(O)Cn1/c(=N/C(=O)c2ccnc(Oc3cccc(O)c3C=O)c2)[nH]c2ccccc21 ; O=Cc1c(O)cccc1Oc1cc(C(=O)/N=c2\[nH]c3ccccc3n2[C@H]2CC[C@@H](O)CC2)ccn1 ; O=Cc1c(O)cccc1Oc1cc(C(=O)/N=c2\[nH]c3ccccc3n2CCN2CCOCC2)ccn1 ; CC(C)(O)Cn1/c(=N/C(=O)c2ccnc(-c3cccc(C=O)c3O)c2)[nH]c2ccccc21 ; CN(C)C/C=C/C(=O)N1CCCC[C@@H](n2c(NC(=O)c3ccnc(-n4cc(-c5cccc(O)c5C=O)cn4)c3)nc3cccc(Cl)c32)C1] 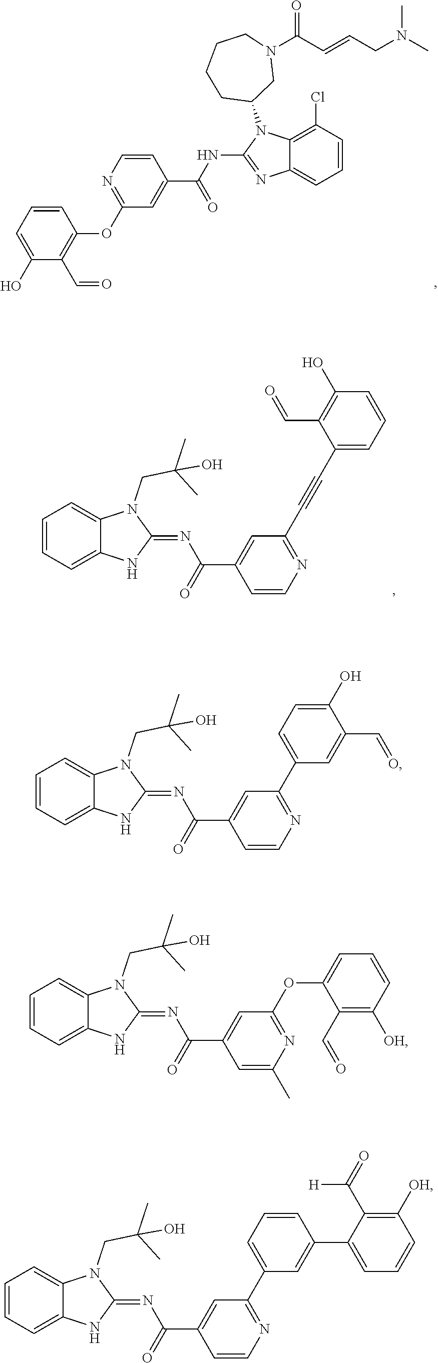 [SMILES: CN(C)C/C=C/C(=O)N1CCCC[C@@H](n2c(NC(=O)c3ccnc(Oc4cccc(O)c4C=O)c3)nc3cccc(Cl)c32)C1 ; CC(C)(O)Cn1/c(=N/C(=O)c2ccnc(C#Cc3cccc(O)c3C=O)c2)[nH]c2ccccc21 ; CC(C)(O)Cn1/c(=N/C(=O)c2ccnc(-c3ccc(O)c(C=O)c3)c2)[nH]c2ccccc21 ; Cc1cc(C(=O)/N=c2\[nH]c3ccccc3n2CC(C)(C)O)cc(Oc2cccc(O)c2C=O)n1 ; CC(C)(O)Cn1/c(=N/C(=O)c2ccnc(-c3cccc(-c4cccc(O)c4C=O)c3)c2)[nH]c2ccccc21] 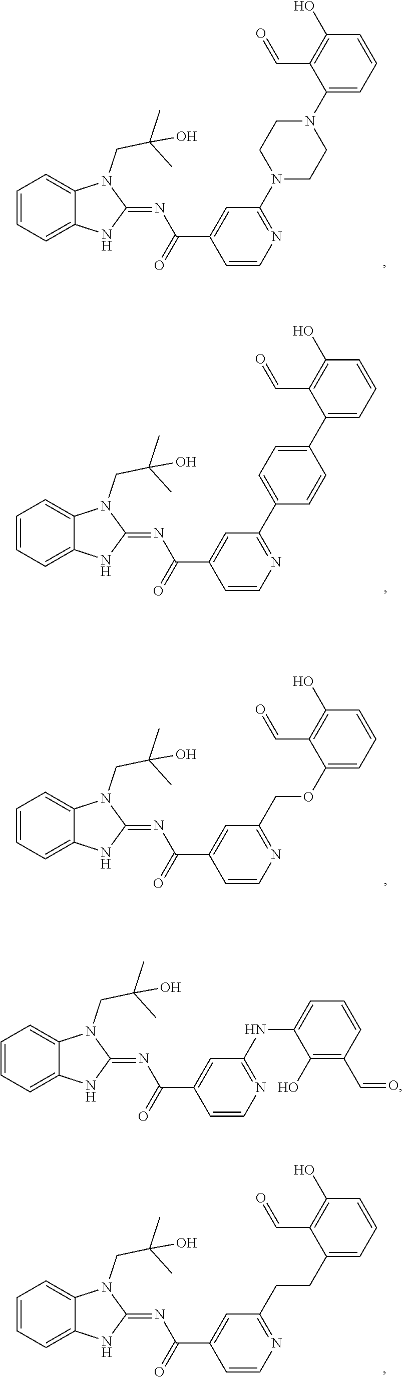 [SMILES: CC(C)(O)Cn1/c(=N/C(=O)c2ccnc(N3CCN(c4cccc(O)c4C=O)CC3)c2)[nH]c2ccccc21 ; CC(C)(O)Cn1/c(=N/C(=O)c2ccnc(-c3ccc(-c4cccc(O)c4C=O)cc3)c2)[nH]c2ccccc21 ; CC(C)(O)Cn1/c(=N/C(=O)c2ccnc(COc3cccc(O)c3C=O)c2)[nH]c2ccccc21 ; CC(C)(O)Cn1/c(=N/C(=O)c2ccnc(Nc3cccc(C=O)c3O)c2)[nH]c2ccccc21 ; CC(C)(O)Cn1/c(=N/C(=O)c2ccnc(CCc3cccc(O)c3C=O)c2)[nH]c2ccccc21] 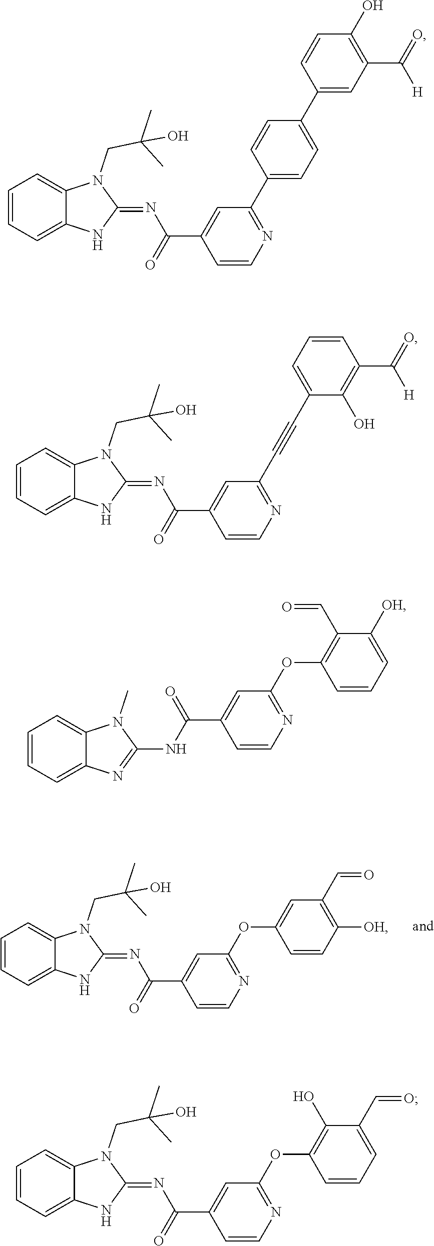 [SMILES: CC(C)(O)Cn1/c(=N/C(=O)c2ccnc(-c3ccc(-c4ccc(O)c(C=O)c4)cc3)c2)[nH]c2ccccc21 ; CC(C)(O)Cn1/c(=N/C(=O)c2ccnc(C#Cc3cccc(C=O)c3O)c2)[nH]c2ccccc21 ; Cn1c(NC(=O)c2ccnc(Oc3cccc(O)c3C=O)c2)nc2ccccc21 ; CC(C)(O)Cn1/c(=N/C(=O)c2ccnc(Oc3ccc(O)c(C=O)c3)c2)[nH]c2ccccc21 ; CC(C)(O)Cn1/c(=N/C(=O)c2ccnc(Oc3cccc(C=O)c3O)c2)[nH]c2ccccc21]
or a pharmaceutically acceptable salt thereof.

198-200. (canceled)
